(19) United States
(12) Patent Application Publication
(10) Pub. No.: US 2009/0258002 A1
(43) Pub. Date: Oct. 15, 2009

(54) Biomarkers for Tissue Status

(75) Inventors: J. Carl Barrett, Shirley, MA (US); Joseph Riss, Bethesda, MD (US)

Correspondence Address: EDWARDS ANGELL PALMER & DODGE LLP, PO BOX 55874, BOSTON, MA, 02205, US

(73) Assignee: Government of the US, as represented by the Secretary, Department of Health and Human Services, Rockville, MD (US)

(21) Appl. No.: 11/883,535

(22) PCT Filed: Feb. 1, 2006
(86) PCT No.: PCT/US06/03611

§ 371 Date: Jun. 16, 2008

Related U.S. Application Data
(60) Provisional application No. 60/649,208, filed on Feb. 1, 2005.

(51) Int. Cl. A61K 39/395; C12Q 1/68; C12Q 1/02; C07H 21/04; C12N 5/06

(52) U.S. Cl. 424/130.1; 435/6; 435/29; 435/375; 536/24.31

(57) ABSTRACT

The invention relates to methods of accurately and quickly diagnosing and monitoring the progression of cancer and ischemally injured tissue. The invention also provides methods of treatment as well as methods of screening for compositions useful for treating the disorders.

### RELATED APPLICATIONS

This application claims priority to U.S. Provisional Patent Application Ser. No. 60/649,208, filed Feb. 1, 2005, entitled “Biomarkers for Tissue Status” and is hereby incorporated by reference in its entirety.

### GOVERNMENT SUPPORT

This work described herein was supported by the National Institutes of Health.

### BACKGROUND OF THE INVENTION

Tumors have been likened to wounds that do not heal, suggesting that tumorogenic processes may share common, or at least analogous, regulatory mechanisms to would healing.

### INTRODUCTION

The processes of tissue regeneration and tumorigenesis are both complex, adaptive processes controlled by cues from the tissue microenvironment. There are various signals that orchestrate a response to injury that results in regeneration and tissue repair of a wound. Tissue regeneration and carcinogenesis both involve processes, such as cell proliferation, survival, and nigration, that are controlled by growth factors, cytokines as well as inflammatory and angiogenic signals. Signals facilitating cell proliferation, survival and invasiveness derive from multiple cellular and extracellular sources in the microenvironment of wounds and cancer. Therefore, wounds and cancer share a number of phenotypes in cellular behavior, signaling molecules, and gene expression. Understanding the similarities between wounds and cancers can reveal new insights into the malignant properties of cancers.

The identification of tumor markers suitable for the early detection and diagnosis of cancer holds great promise to improve the clinical outcome of patients. It is especially important for patients presenting with vague or no symptoms or with tumors that are relatively inaccessible to physical examination. Despite considerable effort directed at early detection, no cost effective screening tests have been developed.

Kidney is a member of a restricted class of organs capable of regeneration and repair following traumatic events such as ischemia/reperfusion injury, which is the major cause of acute renal failure (ARF) in both native (Rabb H and Martin J G 1997) and transplanted kidney (Shoskes D A, and Halloran P F (1996)). In the majority of cases of non-chronic ARF, kidney tissue regenerates and regains complete functionality in the absence of persistent inflammation and fibrosis, even when the initial injury and functional decline are very pronounced (Ysebaert D K et al 2004). The process of renal regeneration and repair (RRR) begins shortly after injury, a period during which necrotic cells are accompanied by replicating cells lining the injured proximal renal tubule. The commitment to DNA synthesis in this population of proliferating cells occurs rapidly, temporally coinciding with the emergence of morphologic and functional derangements. Ischemia/reperfusion injury, regeneration and recovery are part of the same continuum of biological responses and depend on the coordination of the cell-cycle machinery as well as the cells' ability to survive the initial injury (Price P M et al 2004). Clinically and biologically, ischemic ARF is a complex but orderly continuum that can be separated into a series of four overlapping phases that have been referred to as “initiation,” “extension,” “maintenance,” and “recovery” (Sutton T A et al 2002).

Renal cell carcinoma (RCC) accounts for 3% of all adult male malignancies in the United State (Jemal A. et al 2004) and is a clinicopathologically heterogeneous disease that includes several histologically distinct cellular subtypes. A majority of the published evidence suggests that proximal renal tubules are the sites from which malignant RCC cells originate, although a recent study offers evidence that such cells may also originate from distal tubules (Motzer R J et al 1996; Mandriota S J et al 2002). A number of genetic syndromes predispose to the development of RCC, and genes associated with five of these syndromes are identified: von Hippel-Lindau (VHL), met proto-oncogene (MET), fumarate hydratase (FH), Birt-Hgg-Dube syndrome (BHD) and hyperparathyroidism 2 (HRPT2) (Pavlovich and Schmidt 2004). RCC also frequently develops in conjunction with polycystic kidney disease and renal allografts, both of which conditions induce a chronic regenerative response (Brennan et al 1991, Gomez Garcia I et al 2004).

There is a need in the art to understand the similarities between wounds and cancers and for the identification of tumor markers suitable for the detection and diagnosis of the molecular changes in cancers, acute organ failure, wound healing and organ transplantation. There is also a need in the art to develop new therapeutic biomarkers and compositions. Thus, it is desirable to have a reliable and accurate method of determining the renal status in patients, the results of which can then be used to manage their treatment.

### BRIEF SUMMARY OF THE INVENTION

The present invention provides sensitive diagnostic and therapeutic methods using markers for RCC, acute renal failure, RRR, organ transplantation, organ shipment, wound healing, tumors, and organ failure. Also provided are methods for screening for compounds to be used in the therapeutic methods.

The measurement of these markers in patient samples provides information that diagnosticians can correlate with a probable diagnosis of human cancer, ischemia, organ failure, wound healing, tissue regeneration, tissue repair, or a negative diagnosis (e.g., normal or disease-free).

Provided herein are methods of qualifying the tissue status in a subject comprising measuring at least one biomarker in a sample from the subject, wherein the biomarker is selected from the group consisting the markers listed one or more of Tables 7, 8, 9, 13, 20, and 23 and correlating the measurement with tissue status.

In one embodiment, the methods further comprise managing treatment of the subject based on the status, wherein managing treatment is selected from ordering more tests, performing surgery, chemotherapy, dialysis, treatment of acute organ failure, organ transplantation, wound healing treatment, and taking no further action.

In a related embodiment, the methods may further comprise measuring the at least one biomarker after subject management.

In one embodiment, the tissue status is selected from the group consisting of the subject's risk of cancer, regeneration, tissue repair, acute organ failure, organ transplantation, the presence or absence of disease, the stage of disease and the effectiveness of treatment of disease.

In a related embodiment, the methods may further comprise measuring at least two biomarkers in a sample from the subject and correlating measurement of the biomarkers with renal status.

In one embodiment, the biomarkers are selected from one or more of Tables 7, 8, 9, 13, 20, and 23. In a related embodiment, the biomarkers are selected from any one or more of Cluster 1-27. In another related embodiment, the biomarkers are selected from any one or more of discordant genes. In another related embodiment, the biomarkers are selected from any one or more of concordant genes.

The invention provides, in one embodiment, measuring comprising providing a nucleic acid sample from the subject; and capturing one or more of the biomarkers on a surface of a substrate comprising capture reagents that bind the biomarkers. In a related embodiment, the substrate is a nucleic acid chip. In another related embodiment, the nucleic acid chip is an RNA or DNA or oligo-nucleotide chip. In a related embodiment, the substrate is a microtiter plate comprising biospecific affinity reagents that bind the at least one biomarkers and wherein the biomarkers are detected by fluorescent labels.

In one embodiment, the measuring is selected from detecting the presence or absence of the biomarkers(s), quantifying the amount of marker(s), and qualifying the type of biomarker.

The invention provide, in one embodiment, measuring at least one biomarker using a biochip array. In one embodiment, the biochip array is an antibody chip array, tissue chip array, protein chip array, or a peptide chip array. In a related embodiment, the biochip array is a nucleic acid array. In another related embodiment, at least one biomarker capture reagent is immobilized on the biochip array. In yet another related embodiment, the protein biomarkers are measured by immunoassay.

In one embodiment, correlating is performed by a software classification algorithm.

The invention provides, in one embodiment, samples selected from one or more of blood, serum, kidney, renal tumor, renal cyst, renal metastasis, plasma, urine, saliva, and feces. In a related embodiment, the tissue is normal or malignant or ischemic, healing kidney, liver, lung, heart, esophagus, bone, intestine, breast, prostate, brain, uterine cervix, testis, stomach or skin.

In one aspect, the invention provides methods of diagnosing renal status in a subject, comprising determining the pattern of expression of one or more markers listed in one or more of Tables 7, 8, 9, 13, 20, and 23 in a sample from the subject, wherein a differential expression pattern of the one or more markers in a subject is indicative of cancer, acute renal failure, ischemia, or organ transplantation.

In one embodiment, the determining is of any one or more of Trends 1-27. In a related embodiment, the determining is of any one or more of clusters 1-27.

In another aspect, the invention provides methods comprising measuring a plurality of biomarkets in a sample from the subject, wherein the biomarkers are selected from one or more of the group consisting of one or more of Tables 7, 8, 9, 13, 20, and 23 or Clusters 1-27.

According to another aspect, the invention provides kit comprising a capture reagent that binds a biomarker selected from Table 9 or Cluster 1-27 and combinations thereof; and a container comprising at least one of the biomarkers.

In one embodiment, the capture reagent binds a plurality of the biomarkers. In a related embodiment, the capture reagent is a nucleic acid probe. In yet another related embodiment, the kit further comprises a second capture reagent that binds one of the biomarkers that the first capture reagent does not bind.

According to another aspect, a kit is provided comprising a plurality of capture reagents that binds one or more biomarkers selected from Table 9 or Cluster 1-27. In one embodiment, the at least one capture reagent is an antibody or a nucleic acid complementary to the biomarker. In a related embodiment, the kit further comprises a wash solution that selectively allows retention of the bound biomarker to the capture reagent as compared with other biomarkers after washing. In another related embodiment, the kit further comprises instructions for using the capture reagent to detect the biomarker. In one embodiment, the kit detects of one or more of renal cancer, renal regeneration, renal repair, acute renal failure, ischemia or kidney transplantation. In a related embodiment, the instructions provide for contacting a test sample with the capture agent and detecting one or more biomarkers retained by the capture agent.

In one aspect, the invention provides methods of monitoring the treatment of a subject for renal carcinoma, comprising determining one or more pre-treatment expression profiles of markers described in Table 9, in a cell of a subject administering a therapeutically effective amount of a candidate compound to the subject, and determining one or more post-treatment expression profiles of markers described in Table 9, in a cell of a subject, wherein a modulation of the expression profile indicates efficacy of treatment with the candidate compound.

In one embodiment, a pre-treatment expression profile of at least one discordantly or concordantly expressed gene indicates renal carcinoma. In a related embodiment, a post-treatment expression profile of at least one discordantly or concordantly expressed gene indicates the efficacy of the treatment. In another related embodiment, the expression profile is determined by a nucleic acid array method.

In one aspect, the invention provides methods of identification of a candidate molecule to treat renal carcinoma, comprising contacting a cell with a candidate molecule and detecting the expression profile of a target the cell, wherein if the expression profile is of one or more of at least one discordantly and/or concordantly expressed gene the molecule may be useful to treat renal carcinoma, acute renal failure, ischemia, kidney transplantation, organ shipment, cancer or wound healing of regenerative tissues

In one embodiment, the candidate molecule is one or more of a small molecule, a peptide, or a nucleic acid. In a related embodiment, the small molecule is one or more of the molecules listed in Table 9 or Clusters 1-27.

In another embodiment, the method further comprises comparing the expression profile to a standard expression profile. In a related embodiment, the standard expression profile is the corresponding expression profile in a reference cell or population of reference cells. In another related embodiment, the reference cell is one or more cells from the subject, cultured cells, cultured cells from the subject, or cells from the subject pre-treatment.

The invention provides, in one aspect, methods of identifying a diagnostic marker comprising obtaining a sample from an ischemically injured kidney, obtaining a sample from a normal kidney, identifying genes having differential expression in the ischemically injured kidney compared to the normal kidney; and selecting at least one gene as a diagnostic marker for the cancer, acute organ failure, ischemia or organ transplantation.

In one embodiment, the method further comprises obtaining a sample from a cancerous kidney, identifying genes having a differential expression in normal kidney as compared to the cancerous kidney, comparing the genes having an differential expression, identifying genes having an differential expression in the ischemically injured kidney but not in the cancerous kidney; and selecting at least one gene as a diagnostic marker of a cancer of the first cell type.

One aspect provides methods of identifying a gene expression signature in a sample comprising determining the gene expression profile of a sample and comparing the expression profile to Trends 1-27.

In one embodiment, a similar signature to one or more of Trends 1-27 indicates the renal status. In a related embodiment, an inverted signature to one or more of Trends 1-27 indicates similar pathologies, drugs, toxins and conditions inducing cancer, ischemia, regeneration, repair, wound healing, acute organ failure. In another related embodiment, the gene expression signature is used it identify promoters and transcription factors that regulate the differential gene expression signatures listed in Table 9 and Trends 1-27. In yet another related embodiment, a signature that does not correspond to one or more of Trends 1-27 indicates a new trend.

The invention provides, in one aspect, the use of compounds identified according to the methods of certain embodiments and aspects in the treatment of cancer or as anti-cancer drugs, acute renal failure drugs, ischemia drugs, and kidney transplantation drugs.

In one aspect, the invention provides, a bioinformatics tool and method comprising code that accesses data attributed to a sample, the data comprising measurement of at least one biomarker in the sample, the biomarker selected from the group consisting of the markers listed in Table 9 and code that executes a classification algorithm that classifies the renal status of the sample as a function of the measurement.

In one embodiment, the classification algorithm classifies the renal status of the sample as a function of the measurement of a biomarker selected from the group consisting of: the markers listed in Table 9, the markers Cluster 1-27, or Trends 1-27.

In one embodiment, the classification algorithm classifies the renal status of the sample as a function of the measurement of one or more of the biomarkers listed in Table 9, Cluster 1-27, or Trends 1-27.

In one embodiment, the classification algorithm classifies the renal status of the sample as a function of the measurement of one or more of the biomarkers listed in Table 9, Cluster 1-27, or Trends 1-27.

According to one aspect, methods comprising communicating to a subject a diagnosis relating to renal cancer status determined from the correlation of biomarkers in a sample from the subject, wherein said biomarkers are selected from the group consisting of the biomarkers listed in Table 9 or Clusters 1-27 are presented.

In one embodiment, the diagnosis is communicated to the subject via a computer-generated medium.

In one aspect, the invention provides, a method for identifying a candidate compound to treat renal carcinoma, comprising contacting renal carcinoma cancer cell with a test compound and determining the expression profile of one or more of the markers listed in Table 9 in the cancer cell, ischemic cell or the healing cell.

In one embodiment, the candidate compound is generated by the software program and database as PharmaProjects. In another embodiment, the software is any software correlating genes to drug candidates. In a related embodiment, the invention provides methods for screening for combination therapies, e.g., one or more the compounds linked or generated by the software program and database as PharmaProjects (PJP Publications, LTD, England).

In another aspect, the invention provides, methods for modulating the renal profile a cell or group of cells comprising contacting a cell with one or more compounds linked or generated by the software program and database as PharmaProjects or a compound identified in the methods described herein.

In one embodiment, the methods further comprise determining the renal status of the cell or group of cells before the contacting.

In another embodiment, the methods further comprise determining the renal status of the cell or group of cells after the contacting.

In one embodiment, the determining the renal status of the cell is by determining one or more of the expression profiles of the markers listed in Table 9, Cluster 1-27, or Trends 1-27.

According to another aspect, method of treating a condition in a subject comprising administering to a subject a therapeutically effective amount of a compound which modulates a renal profile, wherein a modulation from a renal cell carcinoma profile to a tissue regeneration, tissue repair profile, or a normal profile indicates the efficacy of the treatment is presented.

In one embodiment, the renal profile is measured by gene expression profiling.

In certain embodiments, the methods further comprise managing subject treatment based on the status determined by the method. For example, if the result of the methods of the present invention is inconclusive or there is reason that confirmation of status is necessary, the physician may order more tests. Alternatively, if the status indicates that surgery is appropriate, the physician may schedule the patient for surgery. Likewise, if the result of the test is positive, e.g., the status is late stage renal cancer or if the status is otherwise acute, no further action may be warranted. Furthermore, if the results show that treatment has been successful, no further management may be necessary.

Preferred methods of measuring the biomarkers include use of a biochip array. Biochip arrays useful in the invention include protein and nucleic acid arrays. One or more markers are captured on the biochip array and subjected to laser ionization to detect the molecular weight of the markers. Analysis of the markers is, for example, by molecular weight of the one or more markers against a threshold intensity that is normalized against total ion current. Preferably, logarithmic transformation is used for reducing peak intensity ranges to limit the number of markers detected.

In preferred methods of the present invention, the step of correlating the measurement of the biomarkers with renal status is performed by a software classification algorithm. Preferably, data is generated on immobilized subject samples on a biochip array, by subjecting said biochip array to analysis; and, transforming the data into computer readable form; and executing an algorithm that classifies the data according to user input parameters, for detecting signals that represent markers present in subject and are lacking in non-cancer subject controls.

The markers are characterized by their transcript expression and/or by their known protein identities. The markers can be resolved in a sample by using a variety of techniques, e.g., nucleic acid chips, PCR, real time PCR, reverse transcriptase PCR, real time reverse transcriptase PCR, in situ PCR, chromatographic separation coupled with mass spectrometry, protein capture using immobilized antibodies or by traditional immunoassays.

The invention relates to methods for diagnosing and prognosing cancer, acute renal failure, ischemia, kidney transplantation, tissue regeneration and/or tissue repair by utilizing general as well as tissue-specific genetic markers, methods for identifying these markers, and the markers identified by such methods.

In one aspect, the invention provides methods of diagnosing renal status in a subject comprising determining the pattern of expression of one or more markers listed in Table 9 in a sample from the subject, wherein a differential expression pattern of the one or more markers in a subject free of cancer is indicative of cancer.

In one embodiment, the invention contemplates any of the polynucleotides in Table 6 and polynucleotides that are at least 70% identical to the sequences of the polynucleotides encoding the tumor markers listed in Table 9.

In one aspect, the concordant and discordant gene expression signatures can be used to search global gene expression data bases (e.g., GEO profiles) and datasets for similar signature or inverted signature and as such to identify tumors and pathologies that share the same signature, new drug that will invert the signature, or toxins that can cause cancer or wounds.

In one aspect, provided herein are methods for identifying a candidate compound to treat renal carcinoma, comprising contacting renal carcinoma cancer cell with a test compound; and determining the expression profile of one or more of the markers listed in one or more of Tables 7, 8, 9, 13, 20, or 23 in the cancer cell. In one embodiment, the candidate compound is identified by software program as the software program and database PharmaProjects.

In one aspect, provided herein are methods for modulating the renal profile a cell or group of cells comprising contacting a cell with one or more compounds identified by the software program and data base as PharmaProjects or a compound identified in the method described herein.

In one embodiment, methods may further comprise determining the renal status of the cell or group of cells before the contacting.

In one embodiment, methods may further comprise determining the renal status of the cell or group of cells after the contacting.

In one embodiment, the determining the renal status of the cell is by determining one or more of the expression profiles of the markers listed in one or more of Tables 7, 8, 9, 13, 20, or 23, Cluster 1-27, or Trends 1-27.

In one aspect, provided herein are methods treating a condition in a subject comprising administering to a subject a therapeutically effective amount of a compound which modulates a renal profile, wherein a modulation from a renal cell carcinoma profile to a tissue regeneration, tissue repair profile, or a normal profile indicates the efficacy of the treatment.

In one embodiment, renal profile is measured by gene expression profiling.

In one embodiment, methods may further comprise co-administering a therapeutically effective amount of a second compound which modulates a renal profile.

In one embodiment, the compound is a compound listed in one or more of Tables 7, 8, 9, 13, 20, or 23.

In one aspect, biomarkers for renal status are provided and comprise one or more of the transcripts listed in one or more of Tables 7, 8, 9, 13, 20, or 23.

In one embodiment, the biomarker differentiates tissue regeneration, tissue repair and cancerous tissue from normal tissue.

In one aspect, provided herein are methods method of qualifying the renal status in a subject comprising (a) measuring at least two biomarkers in a sample from the subject, wherein the biomarkers are selected from the group consisting of the markers listed one or more of Tables 7, 8, 9, 13, 20, or 23; and (b) correlating the measurement with renal status.

In one embodiment, methods may further comprise (c) managing treatment of the subject based on the status.

In one embodiment, methods may further comprise (d) measuring the at least one biomarker after subject management.

In one embodiment, the renal status is selected from the group consisting of the subject's risk of cancer, regeneration, tissue repair, acute organ failure, organ transplantation, the presence or absence of disease, the stage of disease and the effectiveness of treatment of disease.

In one embodiment, the biomarkers are selected from any one or more of Cluster 1-27.

In one embodiment, the biomarkers are selected from any one or more of discordant genes.

In one embodiment, the biomarkers are selected from any one or more of concordant genes.

In one embodiment, providing a nucleic acid sample from the subject; and capturing one or more of the biomarkers on a surface of a substrate comprising capture reagents that bind the biomarkers.

In one embodiment, wherein the substrate is a nucleic acid chip.

In one embodiment, the sample is selected from one or more of blood, serum, kidney, renal tumor, renal cyst, renal metastasis, kidney cell or cells, kidney tissue, plasma, urine, saliva, and feces.

In one embodiment, the tissue is kidney tissue.

Other embodiments of the invention are disclosed infra.

### BRIEF DESCRIPTION OF THE DRAWINGS

FIG. 1 depicts is A) as chematic flow of the five-step comparison of global gene expression in RRR and RCC. B. Renal ischemia reperfusion protocol: 5-week-old C57BL/6 female mice were subjected to 50 minutes of left unilateral warm ischemia, followed by reperfusion. Before the ischemia (normal kidney) or after the desired period of reperfusion (0, 6 or 12 h or 1, 2, 5, 7 and 14 days) both kidneys were rapidly excised. Histological studies were carried out for both kidneys. Microarray analysis was carried out using total RNA from the left kidney sampled before or immediately after ischemia or on days 1, 2, 5 and 14 of RRR. C. Venn diagram: 984 genes on the array were previously reported to be differentially expressed in RCC and normal kidney. Comparison with the current microarray study identified 1,325 genes differentially expressed in RCC and normal kidney. 361 genes were differentially expressed in both RRR and RCC. D. Venn diagram of the 361 genes differentially expressed in both RRR and RCC, 278 gene were concordantly expressed, and 83 genes were discordantly expressed. E. Distribution of the 361 genes differentially expressed in both RRR and RCC.

FIG. 2 depicts the results of a histological analysis. The renal ischemia reperfusion started with a damage followed by regeneration and healing.

FIG. 2A-C depict renal tubular injury over the time interval studied. A) Essentially normal murine renal cortex taken at time 0 (H&E, 400×). B) Acute tubular necrosis two days after the ischemic event. About half of the tubules show complete necrosis with loss of epithelium and the remaining tubules show cells with reactive nuclear changes (hyperchromasia, prominent nucleoli) (H&E, 600×). C) Representative renal cortex 14 days after the ischemic event. Most of the tubules show a normal appearance with rare tubules showing degenerative or regenerative changes (H&E, 600×).

FIGS. 2D-G depict Proliferation of renal tubular epithelial cells in response to acute ischemic injury. Sections of mouse kidney were stained with antibody to MiB-1. D) Normal renal cortex at time 0. Only rare tubular cells are positive for MiB-1. E) Renal cortex taken 12 hours after ischemic event. The number of positive cells is similar to that of normal cortex. F) Renal cortex taken at 2 days after the ischemic event. Many tubular epithelial cells now stain positively for MiB-1. G) Renal cortex taken 7 days after ischemic event. Although scattered tubules still show multiple nuclei positive for MiB-1, most tubules are now negative or show rare individual cells with positive staining. (A-D, anti-MiB-1, 600×). FIGS. 2 H-K depict the immunoreactivity for Glut-1. Sections of mouse kidney taken at different time points were stained with antibody to Glut-1. H) Normal-renal cortex taken at time 0. Positive staining is seen mainly in the distal collecting tubules. I) Renal cortex taken at 12 hours after ischemic event. In addition to distal collecting tubules, some proximal tubules are also staining. J) Renal cortex taken at 24 hours after ischemic event. More than half of cortical tubules now show some degree of staining for Glut-1. K) Renal cortex taken at 48 hours after ischemic event. Most tubules are now negative and the staining pattern is similar to that seen at time 0. (A-D, anti Glut-1, 400×).

FIG. 3 depicts the RRR gene expression signature defined three large subsets of early, late and continuously changed genes. A total of 39 kidneys (normal, ischemic, immediately following ischemia and RRR for 1, 2, 5 and 14 days) were each analyzed separately on a microarray. The samples clustered into a dendogram of two parent branches: the first normal and ischemic kidneys and second parent branch of genes continually changed at days 1, 2, 5 and 14 days (*). The second branch clustered further into an early branch (A) that included days 1 and 2 and the late branch (B) that included days 5 and 14 following ischemic renal injury. This figure is an illustration of the dendograms shown in FIGS. 8A-B.

FIG. 4 depicts the gene expression is changed in a timely dependent fashion with multiple trends. The RRR differential gene expressions clustered into 27 trends in a timely dependent fashion, three of which were singletons (supplemented FIG. 10). Here are presented 6 major trends: (A) Trend 5, exhibited 190 genes that were consistently up-regulated from the first day and were still up-regulated at two weeks. These genes involved in the defense response, ECM, cell growth and cell communication; (B) Trend 2, exhibited 194 genes that were up-regulated till the second RRR day, after which the expression started to decline. It includes genes of ribosome, cell death, RNA binding, response to abiotic stimulus, enzyme binding and regulation of cell cycle; (C) Trend 4, exhibited 34 genes that picked on the second RRR, after which the expression decreased back to normal levels. These included genes as ribosomal genes RNA binding, metabolism, intracellular and translational elongation; (D) Trend 1, exhibited 230 genes down regulated genes from the first day and were still down-regulated at two weeks, many of which involved in metabolism and catabolism. (E) Trend 16, exhibited 87 down-regulated genes till the 5^{th} day RRR, where it got back to normal levels. These included genes as calcium ion homeostasis, cell growth and/or maintenance, metal ion homeostasis, cell adhesion and positive regulation of cell proliferation (F) Trend 11, exhibited 46 down-regulated genes till the 5^{th} day RRR, where it started to get back to normal levels. These genes involved in the ion transporter activity, mitochondria. See table 9 for information on the genes and the trends. The data is presented in fold ratios from the normal genes expression.

FIG. 5 depicts the differentially expressed genes in RRR and RCC are regulated similarly. Of the genes whose expression was profiled, 984 genes, printed on the array, were previously described to be differentially expressed in RCC from normal kidney. These genes were qualitatively crossed compared with the current microarray study identifying 1325 RRR differentially expressed genes from normal kidney. 361 genes are expressed in both RRR and RCC (A), 278 concordantly expressed genes and 83 discordantly expressed genes. The data is presented in van diagrams (B). The p value is p<0.05

FIG. 6 depicts the differently expressed genes found in both RRR and RCC exhibited distinct ontologies for concordance and discordance expressed genes and pathways. The functional ontology (Fisher Exact p<0.05) of the differentially expressed genes in both RRR and RCC were crossed compared relative to their expression: concordantly, discordantly, oxygenation and pathways: renal cell culture hypoxia responsive genes vs. normoxia; HIF regulated genes (HRE); VHL, IGF, MYC, NF-kB pathway genes; purine pathway genes; gene expression following renal ischemia reperfusion and/or acute renal failure (ARF) v. normal tissue (A); enlarged are presented ontologies of discordantly expressed genes (B); and discordantly expressed genes (C).

FIG. 7 depicts a molecular interaction map of the RRR-RCC-related pathways in which gene expression differences were observed. A, molecular interaction map. B, summary of symbol definitions. (See Kohn 1999). Although the symbol definitions are independent of color, we have adopted the following color convention to improve clarity. Red, inhibitory interaction; green, stimulatory interaction; purple, transcriptional stimulation; black, binding interaction.

FIG. 8 depicts the RRR gene expression signature defined three large subsets of early, late and continuously changed genes. A total of 39 kidneys (normal, ischemic, immediately following ischemia and RRR for 1, 2, 5 and 14 days) were each analyzed separately on a microarray. The samples clustered into: early RRR differentially expressed genes at days 1 and 2 (A) and late 5 and 14 days (B). The joined cluster was maintained and illustrated in FIG. 3.

FIG. 9 depicts differentially expressed genes were validated by QPCR. The expression of the genes HIF-prolyl hydroxylase 1, 2 and 3 (egln2, egln1 and egln3 respectively) was validated by QPCR. The expression is up-regulated in normal kidney and down-regulated in regenerating kidney.

FIG. 10 depicts the differential gene expressions clustered into 27 trends. The differential gene expressions clustered into 27 trends in a timely dependent fashion, three of which were singletons. In the first set, the cluster of the 27 trends is shown. That is the expression of each gene is plotted.

FIG. 11 depicts the differential gene expressions clustered into 27 trends. The 27 trends are the average differential gene expression of the clusters shown in FIG. 10. The data is presented in fold ratios from the normal genes expression. The identity of the genes in the trends is available in Table 9.

FIG. 12 depicts temporal patterns of gene expression during RRR. A. Principal component analysis of gene expression data during RRR. The first two principal components, PC-1 and PC-2, explain 22.2% and 12.1% of the total variance, respectively. B. The RRR gene expression distribution: 23% of the genes were differentially expressed. The differential gene expression is presented here as up or down in regenerating, as opposed normal or ischemic kidney.

FIG. 13 The differentially expressed genes were clustered according to their pattern of expression as early, late or continually RRR. Functional ontology was analysis performed (p<0.05). The presented ontologies are the ontology core and are hyperlinked to EMBL-EBI. The average RRR expression (log 2) of each ontology is presented in a green to red scale; green down-regulated, red up-regulated. The numbers and average RRR expression of up- and down-regulated genes, the category p-value and enrichment are shown as well. Differentially expressed genes were validated by QPCR. The gene expression of IGFBP1, IGFBP 3, CTGF, AKT, FRAP, MYC, NF-kB, HK1, SIRT7, PHD1, was validated by QPCR. The gene expression of PHD2 and PHD3 was quantified as well

### DETAILED DESCRIPTION OF THE INVENTION

We describe herein, inter alia, novel methods for accurately and quickly diagnosing and monitoring the tissue status, for example renal status. Also described herein are novel methods of screening for drug candidates and for treating patients suffering from cancer or organ injury or subject to organ transplantation.

As described herein, extensive molecular and bioinformatics analysis of renal regeneration and repair in a C57BL/6 mouse model and in human renal carcinoma were done. The analysis of the renal regeneration gene expression signature uncovered three patterns characterized by differential gene expression patterns occurring either early, late, or continuously during kidney regeneration, thereby revealing the complexity of the wound-healing process. Comparison of this gene expression profile with the profile of renal cell carcinoma (RCC) reported in the literature revealed a substantial concordance between the biology of renal regeneration and RCC pathogenesis. The identified discordant pattern differentiating the two processes are useful for identifying cells that are in the process of malignant transformation.

Based on the comparative analysis of these concordant and discordant gene expression patterns, we have identified gene expression programs of pathways, functions, and cellular locations that appear to play a multifaceted role in wound healing and/or carcinogenesis.

The introduction of microarray technology has enabled the characterization and comparison of global gene expression signatures of regenerating and malignant tissues. Recent microarray studies comparing wounds and tumors have provided molecular evidence that keratinocytes at wound margins have gene expression profiles similar to that of squamous cell carcinoma (Pedersen T X et al. 2003). The Brown laboratory at Stanford has recently published a novel in-vitro study characterizing the changes in the global gene-expression profile of fibroblasts exposed to serum, and compared the results with publicly available gene expression data for numerous tumors. The study provides further evidence that a close similarity between the gene expression profile of fibroblasts involved in wound healing process and that characteristic of tumorigenesis exists (Chang H Y et al 2004, Grose R. 2004). Our present study extends these observations to renal regeneration and renal carcinoma, but also for first time examines comprehensively the differences between these two processes.

Kidney is a member of a restricted class of organs capable of regeneration and repair following traumatic events such as ischemia/reperfusion injury, which is the major cause of acute renal failure (ARF) in both native (Rabb H and Martin J G 1997) and transplanted kidney (Shoskes D A, and Halloran P F (1996)). In the majority of cases of non-chronic ARF, kidney tissue regenerates and regains complete functionality in the absence of persistent inflammation and fibrosis, even when the initial injury and functional decline are very pronounced (Ysebaert D K et al 2004). The process of renal regeneration and repair (RRR) begins shortly after injury, a period during which necrotic cells are accompanied by replicating cells lining the injured proximal renal tubule. The commitment to DNA synthesis in this population of proliferating cells occurs rapidly, temporally coinciding with the emergence of morphologic and functional derangements. Ischemia/reperfusion injury, regeneration and recovery are part of the same continuum of biological responses and depend on the coordination of the cell-cycle machinery as well as the cells' ability to survive the initial injury (Price P M et al 2004). Clinically and biologically, ischemic ARF is a complex but orderly continuum that can be separated into a series of four overlapping phases that have been referred to as “initiation,” “extension,” “maintenance,” and “recovery” (Sutton T A et al 2002).

Renal cell carcinoma (RCC) accounts for 3% of all adult male malignancies in the United State (Jemal A. et al 2004) and is a clinicopathologically heterogeneous disease that includes several histologically distinct cellular subtypes. A majority of the published evidence suggests that proximal renal tubules are the sites from which malignant RCC cells originate, although a recent study offers evidence that such cells may also originate from distal tubules (Motzer R J et al 1996; Mandriota S J et al 2002). A number of genetic syndromes predispose to the development of RCC, and genes associated with five of these syndromes have been identified: von Hippel-Lindau (VHL), met proto-oncogene (MET), fumarate hydratase (FH), Birt-Hgg-Dube syndrome (BHD) and hyperparathyroidism 2 (HRPT2) (Pavlovich and Schmidt 2004). RCC also frequently develops in conjunction with polycystic kidney disease and renal allografts, both of which conditions induce a chronic regenerative response (Brennan et al 1991, Gomez Garcia I et al 2004).

The present invention is based upon the discovery that relative to the normal kidney, certain markers are differentially present in samples of renal cancer and in kidney recovering from ischemia and are grouped into two distinct signatures: (1) a substantial concordant overlap reflecting the normal regenerative phenotype, and (2) a divergent discordant (inverted) pattern of expression where gene expression changes are in opposite direction in renal cancer and in kidney recovering from ischemia. Accordingly, the amount of one or more markers found in a test sample compared to a kidney recovering from ischemia, or the presence or absence of one or more markers in the test sample provides useful diagnostic and therapeutic information regarding the renal status of the patient.

### DEFINITIONS

The “initiation phase,” as used herein, refers to the beginning of ischemic ARF. This occurs when renal blood flow decreases to a level resulting in severe cellular ATP depletion, which in turn leads to acute tubular epithelial cell injury and dysfunction of the normal framework of filamentous actin (F-actin) in the cell. Usually, these alterations fall short of being lethal to the cell, but they disrupt the ability of renal tubular epithelial cells and renal vascular endothelial cells to maintain normal renal function. Additionally, the structural abnormalities observed in the renal vasculature during ischemic ARF can be attributed to the ischemic injury to vascular smooth muscle cells and endothelial cells. The inflammatory cascade is initiated in this pattern, possibly by the up-regulation of a variety of chemokines and cytokines that includes IL-1, IL-6, IL 8, monocyte chemoattractant protein-1 (MCP-1), and TNF-alpha. The transcription factor NF-kB is also reported to be up-regulated in the “initiation” phase (Sutton T A et al 2002).

The “extension phase,” as used herein, is ushered in by two major events: continued hypoxia following the initial ischemic event and an inflammatory response. During this phase, cells continue to undergo injury and death, with both necrosis and apoptosis occurring predominantly in the outer medulla. In contrast, the proximal tubule cells of the outer cortex, where blood flow has returned to near-normal levels, undergo cellular repair and improve morphologically. As cellular injury continues in the medullary region during the extension pattern, the glomerular filtration rate continues to fall. There is continued production and release of chemokines and cytokines that further enhance the inflammatory cascade. Based on animal models of renal ischemia, inflammatory cell infiltration in the outer medullary region of the kidney is evident as early as two hours after ischemic injury and is pronounced by 24 hours after the event (Sutton T A et al 2002).

As used herein, “maintenance phase,” refers to the phase when cells undergo repair or apoptosis, proliferate, acquire the ability to migrate, and synthesize ECM proteins to re-establish and maintain the structural integrity of cells and tubules. The glomerular filtration rate becomes stabilized, albeit at a level determined by the severity of the initial traumatic event. This cellular repair and reorganization pattern results in slowly improving cellular function and sets the stage for improvement in organ function. Blood flow approaches normal, and epithelial cells establish intracellular and intercellular homeostasis (Sutton T A et al). During the final “recovery phase” of RRR, cellular differentiation continues, epithelial polarity is re-established, and normal cellular and organ function returns (Sutton T A et al 2002).

Unless defined otherwise, all technical and scientific terms used herein have the meaning commonly understood by a person skilled in the art to which this invention belongs.

The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R Rieger et al. (eds.), Springer Verlag (1991); and Hale & Marham, The Harper Collins Dictionary of Biology (1991). As used herein, the following terms have the meanings ascribed to them unless specified otherwise.

The term “tissue status” refers to the histological status of a tissue sample. For example, diseases state or injury state of the tissue.

The term “renal status” refers to the status of the kidney tissue in a subject. Examples of types of renal statuses include, but are not limited to, the subject's risk of cancer, acute renal failure, the presence or absence of disease, the stage of disease in a patient, and the effectiveness of treatment of disease. Other statuses and degrees of each status are known in the art.

The term “sample” refers to cells, tissue samples or cell components (such as cellular membranes or cellular components) obtained from the treated subject. By one embodiment the sample are cells known to manifest the disease, for example, where the disease is cancer of type X, the cells are the cells of the tissue of the cancer (kidney, etc.) or metastasis of the above. By another embodiment the sample may be non-diseased cells such as cells obtained from a non-involved breast or other tissue.

The sample may be taken from biopsy, a bodily fluid, such as blood, lymph fluid, ascites, serous fluid, pleural effusion, sputum, cerebrospinal fluid, lacrimal fluid, synovial fluid, saliva, stool, sperm and urine. The sample may also originate from a tissue, such as brain, lung, liver, spleen, kidney, pancreas, intestine, colon, mammary gland or kidney, stomach, prostate, bladder, placenta, uterus, ovary, endometrium, testicle, lymph node, skin, head or neck, esophagus, bone marrow, and blood or blood cells. Cells suspected of being transformed may be obtained by methods known for obtaining “suspicious” cells such as by biopsy, needle biopsy, fine needle aspiration, swabbing, surgical excision, and other techniques known in the art. A sample may be tissue samples or cell from a subject, for example, obtained by biopsy, intact cells, for example cell that have been separated from a tissue sample, or intact cells present in blood or other body fluid, cells or tissue samples obtained from the subject, including paraffin embedded tissue samples, proteins extracted obtained from a cell, cell membrane, nucleus or any other cellular component or mRNA obtained from the nucleus or cytosol. As used herein, the “cell from the subject” may be one or more of a renal cell carcinoma, cyst, cortical tubule, ischemic tissue, regenerative tissue, or any histological or cytological stage in-between. The cells are sometimes herein referred to as a sample.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Adsorption” refers to detectable non-covalent binding of an analyte to an adsorbent or capture reagent.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface and, therefore, function as probes.)

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Optical methods of detection include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to quantitative PCR, semi-quantitative PCR, reverse transcriptase PCR, real time PCR, real time reverse transcriptase PCR, in situ PCR, SELDI and immunoassay. For example, PCR may be done using Applied Biosystems MicroFluidic Card. Any suitable methods can be used to detect and measure one or more of the markers described herein. These methods include, without limitation, mass spectrometry (e.g., laser desorption/ionization mass spectrometry), fluorescence (e.g. biochip reader, sandwich immunoassay), radio-isoptoe detection, surface plasmon resonance, ellipsometry and atomic force microscopy.

The phrases “differentially present” and “differentially expressed” refer to differences in the existence, quantity, incidence and/or frequency of a marker present in a sample taken from patients having human cancer as compared to a control subject. A marker can be a nucleic acid or a polypeptide which is detected at a higher frequency or at a lower frequency in samples of human cancer patients compared to samples of control subjects, e.g, a marker may not be present in a normal sample, but may be present in a cancerous sample. A marker can be differentially present in terms of quantity, frequency, existence or incidence, or a combination thereof

A nucleic acid is differentially present between two samples if the amount of the nucleic acid in one sample is statistically significantly different from the amount of the nucleic acid in the other sample. For example, a nucleic acid is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

A biomarker (also referred to herein as a “marker”) is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

Alternatively or additionally, a nucleic acid is differentially present between two sets of samples if the frequency of detecting the nucleic acid in the renal cancer patients' samples is statistically significantly higher or lower than in the control samples. For example, a nucleic acid is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of renal cancer or kidney recovering from ischemia.) A diagnostic amount can be either in absolute amount (e.g., μg/ml) or a relative amount (e.g., relative intensity of signals).

A “control amount” of a marker can be any amount or a range of amount, which is to be compared against a test amount of a marker. For example, a control amount of a marker can be the amount of a marker in a person without renal cancer, a person with ischemic injury, or a primary culture cell line or an established cell line. A control amount can be either in absolute amount (e.g., μg/ml) or a relative amount (e.g., relative intensity of signals).

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

“Managing treatment” refers to the behavior of the clinician or physician subsequent to the determination of renal status. For example, if the result of the methods of the present invention is inconclusive or there is reason that confirmation of status is necessary, the physician may order more tests. Alternatively, if the status indicates that surgery is appropriate, the physician may schedule the patient for surgery. Likewise, if the status is negative, e.g., late stage renal cancer or if the status is acute, no further action may be warranted. Furthermore, if the results show that treatment has been successful, no further management may be necessary.

As used herein, the term “assessing” and “analyzing” are intended to include quantitative and qualitative determination in the sense of obtaining an absolute value for the amount or concentration of the analyte present in the sample, and also of obtaining an index, ratio, percentage, visual and/or other value indicative of the level of analyte in the sample. Assessment may be direct or indirect and the chemical species actually detected need not of course be the analyte itself but may for example be a derivative thereof or some further substance.

The term “modulated” refers to changes in of one or more of the parameters, e.g., the expression of a marker or the level of the expression of a marker.

As used herein, “related clinical intervention” includes chemoprevention and surgical intervention.

“A tumor that responds” refers to a change in the tumor as a result of a treatment, for example, a reduction or stability in growth or invasive potential of the tumor, e.g., a favorable response. A tumor is also considered to respond if it increases or if it becomes more unstable, or exhibits metastasis.

The method may further comprise reporting the expression profile of the marker or markers or the correlations of the expression profiles thereof to the subject or a health care professional. This may be done as a “raw” results that has not been correlated, e.g., as a report of just the determined parameters, or it may be a correlated result.

“Diagnostic,” “diagnosing,” and the like refer to identifying the presence or nature of a pathologic condition, i.e., renal cancer. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “subject” or “patient” are used interchangeably herein, and is meant a mammalian subject to be treated, with human subjects being preferred. In some cases, the methods of the invention find use in experimental animals, in veterinary application, and in the development of animal models for disease, including, but not limited to, rodents including mice, cows, rats, and hamsters, primates, pigs, horses, chickens, cats, or dogs and the like.

The cell from the subject suspected of being cancerous may be anywhere along the progression from normal to neoplastic, including metastatic. For example, such a cell is not normal, and may exhibit signs of displays, or any other pathology between, and including, normal and neoplasia.

The terms “reverse transcription polymerase chain reaction” and “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using a single primer (e.g., an oligo-dT primer) prior to PCR amplification of the desired segment of the transcribed DNA using two primers.

The term “polynucleotide” as used herein refers to a polymeric molecule having a backbone that supports bases capable of hydrogen bonding to typical polynucleotides, where the polymer backbone presents the bases in a manner to permit such hydrogen bonding in a sequence specific fashion between the polymeric molecule and a typical polynucleotide (e.g., single-stranded DNA). Such bases are typically inosine, adenosine, guanosine, cytosine, uracil and thymidine. Polymeric molecules include double and single stranded RNA and DNA, and backbone modifications thereof, for example, methylphosphonate linkages.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and of an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

When determining the levels of transcripts, the transcripts may have the published sequences, or they may be substantially identical to the published sequences due to polymorphisms or mutations.

As used herein, “substantial sequence identity” in the nucleic acid sequence comparison context means either that the segments, or their complementary strands, when compared, are identical when optimally aligned, with appropriate nucleotide insertions or deletions, in at least about 50% of the nucleotides, generally at least 56%, more generally at least 59%, ordinarily at least 62%, more ordinarily at least 65%, often at least 68%, more often at least 71%, typically at least 74%, more typically at least 77%, usually at least 80%, more usually at least about 85%, preferably at least about 90%, more preferably at least about 95 to 98% or more, and in particular embodiments, as high at about 99% or more of the nucleotides. Alternatively, substantial sequence identity exists when the segments will hybridize under selective hybridization conditions, to a strand, or its complement, typically using a fragment derived from the sequences. Typically, selective hybridization will occur when there is at least about 55% sequence identity over a stretch of at least about 14 nucleotides, preferably at least about 65%, more preferably at least about 75%, and most preferably at least about 90%. See Kanehisa (1984) Nuc. Acids Res. 12:203-213. The length of sequence identity comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides. The endpoints of the segments may be at many different pair combinations. In determining sequence identity or percent homology the below discussed protocols and programs for sequence similarity are suitably employed including the BLAST algorithm.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A specific genetic sequence at a polymorphic region of a gene is an allele.

A polymorphic region can be a single nucleotide, the identity of which differs in different alleles. A polymorphic region can also be several nucleotides long. The nucleic acid and protein sequences of the present invention can further be used as a “query sequence” to perform a search against public databases to identify, for example, other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the genes genes listed on table 15 nucleic acid molecules of the invention. BLAST protein searches can be performed with the XBLAST program, score=50, wordlength=3 to obtain amino acid sequences homologous to NIP2b, NIP2cL, and NIP2cS protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

Sequence identity searches can be also performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. The GCG Package provides a local version of Blast that can be used either with public domain databases or with any locally available searchable database. GCG Package v9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the Internet at, for example, http://www.gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for sequence identity searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence identity search can be performed, for example, by BlastParse. BlastParse is a PERL script running on a UNIX platform that automates the strategy described above. BlastParse takes a list of biomarker accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

As used herein, the term “specifically hybridizes” or “specifically detects” refers to the ability of a nucleic acid molecule to hybridize to at least approximately 6 consecutive nucleotides of a sample nucleic acid.

“Substantially purified” refers to nucleic acid molecules or proteins that are removed from their natural environment and are isolated or separated, and are at least about 60% free, preferably about 75% free, and most preferably about 90% free, from other components with which they are naturally associated.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

A nucleic acid derived from a biomarker is one derived from at least the C-terminal 100 nucleic acids, 75 nucleic acids, 50 nucleic acids, 25 nucleic acids, 10 nucleic acids, or 5 nucleic acids. Alternately, the isolated nucleic acid has a sequence corresponding to the amino acid sequence as identified by the sequences, or fragments or variants thereof. Nucleic acids of the invention may be at least about 60%, 70%, 75%, 80%, 85%, 90%, 95%, or 99.9% identical to the nucleotide sequence identified by the sequences, fragments or variants thereof, or one that is identified in a screening assay descried herein. Nucleic acids may also be those capable of encoding a polypeptide having substantial sequence identity to the sequence identified by the sequences, fragments or variant thereof, and characterized by the ability to alter the expression pattern of a biomarker. Nucleic acids of the invention may be at least about 60%, 70%, 75%, 80%, 85%, 90%, 95%, or 99.9% identical to the nucleic acids capable of encoding a polypeptide having substantial sequence identity to those identified by the screening assays described herein, fragments or variant thereof, and characterized by the ability to alter the expression pattern of a biomarker.

An isolated polypeptide, of the invention, may be a peptide derived from a biomarker, wherein the polypeptide stimulates an alternation in the subcellular expression pattern of a biomarker. The peptide may be an amino acid sequence as identified by the sequences, or fragments or variants thereof. The peptide is at least about 60%, 65%, 70%, 75%, 80%, 85%, 90%, 95%, or 99% identical to any one or more of the amino acid sequences identified by the sequences. The peptide may also be a peptide identified by the screening methods described herein or fragments or variants thereof. For example, the peptide may be a peptide that is at least about 60%, 65%, 70%, 75%, 80%, 85%, 90%, 95%, or 99% identical to any one or more of the amino acid sequences identified by a screening method described herein.

As used herein, the term “an oligonucleotide having a nucleotide sequence encoding a gene” means a nucleic acid sequence comprising the coding region of a gene, i.e. the nucleic acid sequence which encodes a gene product. For example, the sequences is an oligonucleotide encoding a c-terminal portion of the a biomarker gene. The coding region may be present in either a cDNA, genomic DNA or RNA form. When present in a DNA form, the oligonucleotide may be single-stranded (e.g., the sense strand) or double-stranded. Suitable control elements such as enhancers, promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers, splice junctions, intervening sequences, polyadenylation signals, etc. or a combination of both endogenous and exogenous control elements.

The terms “protein” and “polypeptide” are used interchangeably herein. The term “peptide” is used herein to refer to a chain of two or more amino acids or amino acid analogs (including non-naturally occurring amino acids), with adjacent amino acids joined by peptide (—NHCO—) bonds. Thus, the peptides of the invention include oligopeptides, polypeptides, proteins, mimetopes and peptidomimetics. Methods for preparing mimetopes and peptidomimetics are known in the art.

The terms “mimetope” and “peptidomimetic” are used interchangeably herein. A “mimetope” of a compound X refers to a compound in which chemical structures of X necessary for functional activity of X have been replaced with other chemical structures which mimic the conformation of X. Examples of peptidomimetics include peptidic compounds in which the peptide backbone is substituted with one or more benzodiazepine molecules (see e.g., James, G. L. et al. (1993) Science 260:1937-1942) and “retro-inverso” peptides (see U.S. Pat. No. 4,522,752 to Sisto). The terms “mimetope” and “peptidomimetic” also refer to a moiety, other than a naturally occurring amino acid, that conformationally and functionally serves as a substitute for a particular amino acid in a peptide-containing compound without adversely interfering to a significant extent with the function of the peptide. Examples of amino acid mimetics include D-amino acids. Peptides substituted with one or more D-amino acids may be made using well known peptide synthesis procedures. Additional substitutions include amino acid analogs having variant side chains with functional groups, for example, b-cyanoalanine, canavanine, djenkolic acid, norleucine, 3-phosphoserine, homoserine, etc.

“Discordant genes” refer to genes that are expressed in a divergent discordant (inverted) pattern of expression where gene expression changes are in opposite direction in cancer and normal tissue recovering from ischemia, by going through the processes of regeneration and repair, (e.g., kidney). Discordantly expressed genes include the genes labeled as discordantly expressed in Table 9. Discordant genes, as disclosed herein, are useful for diagnosing, treating or screening for candidate compounds to treat cancer and to aid in wound healing. For example, kidney cancer and wound healing (i.e. acute renal failure and kidney transplantation). The discordant pattern of expression could also be used to treat cancer and wound healing in brain, lung, liver, spleen, kidney, pancreas, intestine, colon, mammary gland or kidney, stomach, prostate, bladder, placenta, uterus, ovary, endometrium, testicle, lymph node, skin, head or neck, esophagus. It could also be used to treat cancer, metastasis, cyst, wound healing and ischemia of heart, lung, esophagus, bone, intestine, breast, brain, uterine cervix, testis, stomach, skin, and organs that are transplantable. For example, discordant gene expression patterns and signatures could be used to identify drugs that will slow the ischemia when shipping organs (e.g., live donors will be given drug and/or the transplanted organ will be treated with the same or different drugs). That is, divergent, discordant (inverted) pattern of expression is where gene expression changes are in the opposite direction in RRR and RCC. The RRR differential gene expression was qualitatively compared with the global gene expression of RCC as opposed to human normal kidney. Two distinct signatures were revealed: (1) a substantial concordant overlap reflecting the normal regenerative phenotype, and (2) a divergent discordant (inverted) pattern of expression where gene expression changes are in opposite direction in RRR and RCC. The RCC/normal tissue profile and the RRR/normal tissue profile was compared. Qualitative cross-comparison, e.g., “A”/“B”=RCC/RRR. The RCC/RRR produced two subgroups, e.g., concordant genes (up or down regulated from normal in both RCC and RRR) and discordant genes (up regulated from normal in RCC and down regulated in RRR, or the other way round). Discordant genes can be used to diagnose and or treat cancer, wound healing, RRR, acute organ failure, organ transplantation.

“Clusters,” as used herein refer to patterns of gene expression that are similar. For example, three patterns of differentially expressed genes were categorized during days 1-14 of Renal Regeneration and Repair (RRR): continuous, early and late. “Trends,” refer to the averages of the identified clusters. The RRR differential gene expression as compared to normal kidney was further clustered to identify different temporal trends over the two-week period. We statistically identified 27 trends that are described in details in the supplemental material

BRB tools may be used to statistically identify clusters and trends. See http://linus.nci.nih.gov/BRB-ArrayTools.html.

“Gene Ontology (GO)” analysis can be done, for example, using the EASE software. Significant ontology for the three patterns of gene expression (continuous, early and late) were identified using EASE.

PubMed and other publicly available databases were searched to catalogue differentially regulated genes relative to the normal kidney/tissue for at least the following conditions or statuses: renal cell carcinoma (RCC), acute renal failure (ARF) and RRR, hypoxia, hypoxia inducible factor (HIF), (HIF binds to the Hypoxia Responsive Element (HRE) in the promoter of many genes), the VHL gene, the MYC gene, the p53 gene, the NF-kB gene, and the IGF gene. The datasets (catalogues) of the conditions or statutes were cross-compared with a microarray dataset of 1325 RRR genes. The significance of these cross-comparisons was also tested (x2 test).

“Concordant genes” refer to genes that reflect the normal regenerative phenotype. Concordant genes are up-regulated from normal in both RRR and RCC or down-regulated in both. Discordant genes are up-regulated from normal in RRR but down-regulated in RCC or the other way round. Concordant may also refer to genes or proteins differentially expressed in the same direction in RRR and RRC. Without wishing to be bound by any particular scientific theory, the concordant signatures qualitatively reflects the regenerative phenotype and discordant signatures reflect differences between malignancies and processes of tissue repair.

“Cosmetics” as used herein refer to ointments, powders, lotions, salves, and the like that are used by subjects on the skin. Compounds identified here can be added to cosmetics to treat wounds to the skin.

“Metastasis” as used herein indicates migrating tumor cells. The discordant and/or concordant gene profiles are useful for treating metatasis, e.g., renal metastasis and for screening for drugs to treat such metastasis.

“Renal cell carcinoma (RCC)” refers to a types of kidney cancer. Other kidney tumors are also included here, for example, Wilms tumors (WT), Birt-Hogg-Dube' (BHD), and hereditary papillary renal-cell carcinoma (HPRC).

### Description of The Biomarkers

### Concordant Biomarker: Mini-Chromosome Maintenance (Mcm2, 3, 4 and 7) And Discordant Biomarker Vascular Endothelial Growth Factor (VEGF)

One example of a marker that is useful in the methods of the present invention include the markers listed in one or more of Tables 7, 8, 9, 13, 20, and 23. The markers were detected by extensively surveying the literature and cataloging 2815 genes expressed differentially in RCC as relative to normal kidney. 984 of these genes were printed on the GEM2 array that we used for the RRR studies. Then RCC dataset was qualitatively cross-compared with the differential expression of the current set of 1,325 RRR genes as relative to normal kidney. The analysis revealed a group of 361 genes that matched both the experimental RRR dataset and the RCC literature. Of these 361 genes, 285 genes (77%) were concordantly expressed in both RRR and in RCC. The remainder of the 361 genes, 81 genes (23%), were discordantly expressed during RRR as compared to RCC. The protocols for isolating and identifying the markers described in one or more of Tables 7, 8, 9, 13, 20, and 23 and elsewhere herein are set forth below in the Examples.

A biomarker can be detected by any methodology. A preferred method for detection involves first capturing the biomarker, e.g., with biospecific capture reagents, and then detecting the captured biomarkers, e.g., nucleic acids with fluorescence detection methods or proteins by mass spectrometry. Preferably, the biospecific capture reagents are bound to a solid phase, such as a bead, a plate, a membrane or a chip. Methods of coupling biomolecules, such as nucleic acids and antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations.

In yet another embodiment, the surfaces of biochips can be derivatized with the capture reagents in the same location or in physically different addressable locations. One advantage of capturing different markers in different addressable locations is that the analysis becomes simpler.

### Types of Sample and Preparation of the Sample

The markers can be measured in different types of biological samples. The sample is preferably a biological cell or fluid sample. Examples of a biological cell samples include kidney cell, e.g., proximal renal tubule (PRT) cells, distal renal tubule (DRT) cells. Examples of a biological fluid sample useful in this invention include blood, blood serum, plasma, vaginal secretions, urine, tears, saliva, etc.

If desired, the sample can be prepared to enhance detectability of the markers. For example, the mRNA may be enriched in an RNA preparation from a cell sample. In fluid samples, such as a blood serum sample from the subject can be preferably fractionated by, e.g., Cibacron blue agarose chromatography and single stranded DNA affinity chromatography, anion exchange chromatography, affinity chromatography (e.g., with antibodies) and the like. The method of fractionation depends on the type of detection method used.

Any method that enriches for the nucleic acid or protein of interest can be used. Sample preparations, such as pre-fractionation protocols, are optional and may not be necessary to enhance detectability of markers depending on the methods of detection used. For example, sample preparation may be unnecessary if antibodies that specifically bind markers are used to detect the presence of markers in a sample.

Optionally, a marker can be modified before analysis to improve its resolution or to determine its identity. For example, the markers may be subject to proteolytic or endonuclease digestion before analysis. Any protease or endonuclease can be used. Proteases, such as trypsin, that are likely to cleave the markers into a discrete number of fragments are particularly useful.

### Data Analysis

When the sample is measured and data is generated, e.g., by mass spectrometry, the data is then analyzed by a computer software program. Generally, the software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human cancer and determines the closeness of fit between the two signals. The software also can include code indicating which the test sample is closest to, thereby providing a probable diagnosis.

In preferred methods of the present invention, multiple biomarkers are measured. The use of multiple biomarkers increases the predictive value of the test and provides greater utility in diagnosis, toxicology, patient stratification and patient monitoring. The process called “Pattern recognition” detects the patterns formed by multiple biomarkers greatly improves the sensitivity and specificity of clinical proteomics for predictive medicine. Subtle variations in data from clinical samples, e.g., obtained using SELDI, indicate that certain patterns of protein expression can predict phenotypes such as the presence or absence of a certain disease, a particular stage of cancer progression, or a positive or adverse response to drug treatments.

Baseline subtraction improves data quantification by eliminating artificial, reproducible instrument offsets that perturb the spectrum. Methods of subtracting baseline are well known in the art.

In one example, GenePix software, Axon Instruments, now part of Molecular Devices USA, is used to detect the results from the biochip. The data is classified using a pattern recognition process that uses a classification model. The statistical analysis was done on the statistical software BRB ArrayTools developed by Dr. Richard Simon and Dr. Amy Peng Lam, NCI, NIH, USA. BRB ArrayTools is an integrated package for the visualization and statistical analysis of DNA microarray gene expression data. It was developed by professional statisticians experienced in the analysis of microarray data and involved in the development of improved methods for the design and analysis of microarray based experiments. The array tools package utilizes an Excel front end. Scientists are familiar with Excel and utilizing Excel as the front end makes the system portable and not tied to any database. The input data is assumed to be in the form of Excel spreadsheets describing the expression values and a spreadsheet providing user specified phenotypes for the samples arrayed. The analytic and visualization tools are integrated into Excel as an add-in. The analytic and visualization tools themselves are developed in the powerful R statistical system, in C and Fortran programs and in Java applications. Visual Basic for Applications is the glue that integrates the components and hides the complexity of the analytic methods from the user. The system incorporates a variety of powerful analytic and visualization tools developed specifically for microarray data analysis.

Other software that were used are Microsoft Excel, FilemakerPro, Michael Eisen Cluster, EASE (Hosack D A et al 2003), GoMiner (Zeeberg B R et al 2003), Source (Diehn M. et al 2003) MatchMiner (Bussey et al 2003) and the p-value for the 2×2 table was calculated using Statistic Package R.

Classification models, e.g., to generate trends and clusters, can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

### Methods

Methods of determining the expression pattern of a polynucleotide in a sample are well known in the art and include, for example, RT-PCR analysis, in-situ hybridization and northern blotting; polynucleotide detection may also be performed by hybridizing a sample with a microarray imprinted with markers. Any other known methods of polynucleotide detection are also envisaged in connection with the invention. Optimization of polynucleotide detection procedures for diagnosis is well known in the art and described herein below. Specifically, diagnostic assays using the above methods are well known in the art (see, for example: Sidransky, “Nucleic Acid-Based methods for the Detection of Cancer”, Science, 1997; 278: 1054-1058) and may be carried out essentially as follows: RT-PCR for diagnosis may be carried out essentially as described in Bernard & Wittwer, “Real-Time PCR Technology for Cancer Diagnostics”, Clinical Chemistry 2002; 48(8): 1178-85; Raj et al., “Utilization of Polymerase Chain Reaction Technology in the Detection of Solid Tumors”, Cancer 1998; 82(8): 1419-1442; Zippelius & Pantel, “RT-PCR-based detection of occult disseminated tumor cells in peripheral blood and bone marrow of patients with solid tumors. An overview”, Ann NY Acad Sci 2000; 906:110-23. In-situ hybridization for diagnosis may be carried out essentially as described in “Introduction to Fluorescence In Situ Hybridization: Principles and Clinical Applications”, Andreeff & Pinkel (Editors), John Wiley & Sons Inc., 1999; Cheung et al., “Interphase cytogenetic study of endometrial sarcoma by chromosome in situ hybridization, modern Pathology 1996; 9:910-918. Northern blotting for diagnosis may be carried out essentially as described in Trayhurn, “Northern blotting”, Proc Nutr Soc 1996; 55(1B): 583-9; Shifman & Stein, “A reliable and sensitive method for non-radioactive Northern blot analysis of nerve growth factor mRNA from brain tissues”, Journal of Neuroscience Methods 1995; 59: 205-208; Pacheco et al., “Prognostic significance of the combined expression of matrix metalloproteinase-9, urokinase type plasminogen activator and its receptor in renal cancer as measured by Northern blot analysis”, Int J Biol Markers 2001; 16(1): 62-8. Polynucleotide microarray-based diagnosis can be carried out essentially as described in Ring & Boss, “Microarrays and molecular markers for tumor classification”, Genuine Biol 2002; 3(5): comment 2005; Lacroix et al., “A low-density DNA microarray for analysis of markers in renal cancer”, Int J Biol Markers 2002; 17(1): 5-23. In addition, polynucleotide microarray hybridization for diagnosis may be carried out essentially as described in the following review concerning micorarrays in the diagnosis of various cancers: Schmidt & Begley, “Cancer diagnosis and microarrays”, The International Journal of Biochemistry and Cell Biology, 2003; 35: 119-124. Diagnostic assays using tissue microarrays are also possible and may be performed essentially as described in Ginestier et al., “Distinct and complementary information provided by use of tissue and DNA microarrays in the study of kidney tumor markers”, Am J Pathol 2002; 161(4): 1223-33; Fejzo & Slamon, “Frozen tumor tissue microarray technology for analysis of tumor RNA, DNA and proteins”, Am J Pathol 2001; 159(5): 1645-50.

An example of detection of polynucleotides in bodily fluid is that of expression profile determination or marker determination, which is diagnostic of the stage of a cancer by detection of the presence of specific cancer cells by RT-PCR of identified cancer-type-specific markers expression in the sample.

Any of the diagnostic methods as described above can also be used together, simultaneously or not, and can thus provide a stronger diagnostic tool and validate or strengthen the results of a particular diagnosis. For combinations of different diagnostic methods see, inter alia: Hoshi et al., Enzyme-linked immunosorbent assay detection of prostate-specific antigen messenger ribonucleic acid in prostate cancer”, Urology 1999; 53 (1): 228-235; Zhong-Ping et al., “Quantitation of ERCC-2 Gene Expression in Human Tumor Cell Lines by Reverse Transcription-Polymerase Chain Reaction in Comparison to Northern Blot Analysis”, Analytical Biochemistry 1997; 244: 50-54; Hatta et al., “Polymerase chain reaction and immunohistochemistry frequently detect occult melanoma cells in regional lymph nodes of melanoma patients”, J Clin Pathol 1998; 51(8): 597-601.

Methods of diagnosing a cancer in a subject comprise determining, in a sample from the subject, the expression profile at least one marker (nucleic acid or protein), wherein an expression pattern as identified in Table 9 is indicative of the renal status.

General protocols for the detection of cancer markers can be found in “Tumor Marker Protocols”, Hanausek & Walaszek (Eds.), Humana Press, 1998. Methods of determining the expression pattern of a polypeptide in a sample are well known in the art (see, for example: Coligan et al, Unit 9, Current Protocols in Immunology, Wiley Interscience, 1994) and include, inter alia: immunohistochemistry (Microscopy, Immunohistochemistry and Antigen Retrieval Methods For Light and Electron Microscopy, M. A. Hayat (Author), Kluwer Academic Publishers, 2002; Brown C.: “Antigen retrieval methods for immunohistochemistry”, Toxicol Pathol 1998; 26(6): 830-1; ELISA (Onorato et al., “Immunohistochemical and ELISA assays for biomarkers of oxidative stress in aging and disease”, Ann NY Acad Sci 1998 20; 854: 277-90), western blotting (Laemmeli UK: “Cleavage of structural proteins during the assembly of the head of a bacteriophage T4”, Nature 1970; 227: 680-685; Egger & Bienz, “Protein (western) blotting”, Mol Biotechnol 1994; 1(3): 289-305), antibody microarray hybridization (Huang, “detection of multiple proteins in an antibody-based protein microarray system, Immunol Methods 2001 1; 255 (1-2): 1-13) and Biomarkered molecular imaging, which can be carried out on the whole body with imaging agents such as antibodies against the marker polypeptides (which may be membrane-bound proteins), the marker polypeptides themselves, receptors and contrast agents. The visualizations techniques include single photon and positron emission tomography, magnetic resonance imaging (MRI), computed tomography or ultrasonography (Thomas, Biomarkered Molecular Imaging in Oncology, Kim et al (Eds)., Springer Verlag, 2001). Any other known methods of polypeptide detection are also envisaged in connection with the invention. Optimization of protein detection procedures for diagnosis is well known in the art and described herein below. Specifically, diagnostic assays using the above methods may be carried out essentially as follows: Immunohistochemistry for diagnosis may be carried out essentially as described in Diagnostic Immunohistochemistry, David J., MD Dabbs, Churchill Livingstone, 1st Ed, 2002; Quantitative Immunohistochemistry: Theoretical Background and its Application in Biology and Surgical Pathology, Fritz et al., Gustav Fischer, 1992. Western blotting-based diagnosis may be carried out essentially as described in Brys et al., “p53 protein detection by the Western blotting technique in normal and neoplastic specimens of human endometrium”, Cancer Letters 2000; 148 (197-205); Rochon et al., “Western blot assay for prostate-specific membrane antigen in serum of prostate cancer patients” Prostate 1994; 25(4): 219-23; Dalmau et al., “Detection of the anti-Hu antibody in the serum of patients with small cell lung cancer—a quantitative western blot analysis”, Ann Neurol 1990; 27(5): 544-52; Joyce et al., “Detection of altered H-ras proteins in human tumors using western blot analysis”, Lab Invest 1989; 61(2): 212-8. ELISA based diagnosis may be carried out essentially as described in D'ambrosio et al., “An enzyme-linked immunosorbent assay (ELISA) for the detection and quantitation of the tumor marker 1-methylinosine in human urine”, Clin Chim Acta 1991; 199(2): 119-28; Attalah et al., “A dipstick, dot-ELISA assay for the rapid and early detection of bladder cancer”, Cancer Detect Prev 1991; 15(6): 495-9; Erdile et al., “Whole cell ELISA for detection of tumor antigen expression in tumor samples”, Journal of Immunological. Methods 2001; 258: 47-53. Antibody microarray-based diagnosis may be carried out essentially as described in Huang, “detection of multiple proteins in an antibody-based protein microarray system, Immunol Methods 2001 1; 255 (1-2): 1-13. Biomarkered molecular imaging-based diagnosis may be carried out essentially as described in Thomas, Biomarkered Molecular Imaging in Oncology, Kim et al (Eds)., Springer Verlag, 2001; Shahbazi-Gahrouei et al., “In vitro studies of gadolinium-DTPA conjugated with monoclonal antibodies as cancer-specific magnetic resonance imaging contrast agents”, Australas Phys Eng Sci Med 2002; 25(1): 31-8; Tiefenauer et al., “Antibody-magnetite nanoparticles: in vitro characterization of a potential tumor-specific contrast agent for magnetic resonance imaging”, Bioconjug Chem 1993; 4(5): 347-52; Cerdan et al., “Monoclonal antibody-coated magnetite particles as contrast asents in magnetic resonance imaging of tumors”, Magn Reson Med 1989; 12(2): 151-63. In addition, polypeptides may be detected and a diagnostic assay performed using Mass Spectrometry, essentially as described in Bergquist et al., “peptide mapping of proteins in human body fluids using electrospray ionization fourier transform ion cyclotron resonance mass spectrometry”, Mass Spectrometry Reviews, 2002; 21:2-15 and Gelpi, “Biomedical and biochemical applications of liquid-chromatography-mass spectrometry”, Journal of Chromatography A, 1995; 703: 59-80.

The diagnostic methods of the invention as recited herein may also be employed to examine the status of a tumor cell or cells, or to examine the effectiveness of a modulator of the activity of a tumor cell, such as a drug. The examining may be by measuring the expression pattern of one or more of the transcripts and/or proteins listed in any one of Tables 8 or 9. The drug may be any one or more of the drugs linked or generated by the software program and database as PharmaProjects and/or a compound or composition identified in a screening assay described herein.

A prognostic aspect of the invention provides a method of measuring the responsiveness of a subject to a cancer treatment comprising determining the expression profile of at least one marker in a sample taken from the subject before treatment, and comparing it with the expression profile of the marker in a sample taken from the subject after treatment. An expression pattern of a marker as listed in Table 9 indicating responsiveness of the subject to the cancer treatment, wherein the marker is selected from the group consisting of: markers listed in Table 9.

In addition, a prognostic aspect of the invention may further comprise methods of measuring the responsiveness of a subject to a cancer treatment comprising determining the expression profile of at least one transcript in a sample taken from the subject before treatment, and comparing it with the expression profile of the polynucleotide in a sample taken from the subject after treatment.

In accordance with the prognostic aspect of the invention, the treatment in conjunction with which the above methods of measuring the responsiveness of a subject to a cancer treatment may be employed include, for example, radiotherapy, surgical treatment, chemotherapy, and the like.

The methods disclosed herein may also be indicative of the status of a biomarker gene, as described above. Where a biomarker gene or a pathway in which such gene is involved is defective or abnormal, this information may also serve in prognosis of both disease progression and treatment responsiveness of a patient, regardless of whether said treatment is directed to the biomarker in question.

Methods for the identification of marker gene biomarkers for both diagnostic and therapeutic applications in any given cancer type. In certain embodiments, these methods use a combination of recently developed powerful functional gene cloning methodologies with cDNA array-based gene expression profiling and rationally designed experimental models. Diagnostic and therapeutic value of the identified genes may then be evaluated using specific inhibitors and antibodies according to methods well known to those of skill in the art.

By identifying those genes that are specifically upregulated (or indeed down-regulated) in cancer cells as a result of biomarker regulation, the invention provides markers of advanced stages of cancer. More specifically, the invention relates to identifying potential biomarkers of biomarker regulation associated with early and advanced stages of the disease by performing micro-array hybridization and analyses using model cancer cell line(s) or primary normal cell cultures that retain wild-type biomarker activity and engineering a variant of such a cell line or primary cells in which the biomarker is inactivated. Alternatively, the tissue pairs for comparison will be normal animal tissues and the same cancer-free tissues from genetically modified animals in which a biomarker gene of interest was knocked out.

The methods of the invention generally provide a systematic approach for the search of cancer markers or biomarkers for therapeutic intervention among the genes normally under control of biomarker proteins. These biomarker can be expressed discordantly or concordantly between RRR and RCC. If expressed concordantly it will reflect a gene expression which is conserved between cancer and wound healing and represent a therapeutic target which permits the tumor to respond to certain physiological signals that are known inhibit tissue regeneration. A discordantly expressed gene represent a divergent discordant (inverted) pattern of expression where gene expression changes are in opposite direction in RRR and RCC. Thus the discordant gene expression is marker for diagnostics and therapeutics of renal carcinoma or wound healing.

The methods of the invention may be performed by comparing gene expression profiles of the markers in cell lines or tissues.

An exemplary model for the screening methods of the invention is the ischemic/reperfusion injury model in rodents.

Selection of cancer or wound healing diagnostic markers, the following criteria were applied:
(1) genes that are concordantly expressed in RCC and RRR are useful as drug targets which permits the tumor or the wounded tissue to respond to certain physiological signals that are known inhibit or induce tissue regeneration,
(2) genes that are discordantly expressed in RCC and RRR are useful as diagnostic targets which distinct to these tumor or wound healing.
(3) genes that are discordantly expressed in RCC and RRR are useful as drug targets which permits the tumor or the wounded tissue to respond to certain physiological signals that are distinct to tumor or the wounded tissue, but not for both.

The genes identified in Table 1-13 are useful in diagnostic and prognostic application as well as act as drug biomarkers for therapeutic intervention of the diseased state.

### Diagnostic Methods of Using Identified Markers

In the genetic diagnostic applications of the invention, one of skill in the art would detect variations, modulations, discordance, or concordance in the expression of one or more of the markers. This may comprise determining the mRNA level or expression patterns of the gene(s) or determining specific alterations in the expressed gene product(s). The cancers that may be diagnosed according to the invention include cancers of kidney or other tissue.

Discordant genes, as described herein and listed in Table 9, are expressed discordantly in RCC from RRR. The discordant signature can be used as a diagnostic and screening assays for kidney cancer and wound healing (i.e. acute renal failure and kidney transplantation). Discordant gene expression analysis can also be used to diagnose ischemia, for example when shipping organs. The discordant signature or pattern of gene expression can be used to identify drugs and drugs combinations for use in anti cancer application and/or in slowing ischemia when shipping organs (i.e., if live donor, she/he will get the drug or the kidney will be treated with such drugs).

This method and data be useful for diagnosing and treatment of cancer or ischemia and wound healing in liver, lung, heart, esophagus, bone, intestine, breast, brain, uterine cervix, testis, stomach, prostate, or skin. Specifically in ischemia, acute renal failure renal, renal regeneration and repair, cyst, renal metastasis, renal cancers this method could be used in renal cell carcinoma, Wilms tumors (WT), Birt-Hogg-Dube' (BHD), and hereditary papillary renal-cell carcinoma (HPRC).

Nucleic acids can be isolated from cells contained in the biological sample, according to standard methodologies (Sambrook et al., 1989). The nucleic acid may be whole RNA, a mixture of RNA and DNA, mRNA, poly-A RNA, and the like. The nucleic acid sample, e.g. RNA, may be used for Northern blotting analysis or may be converted to a complementary DNA (cDNA). cDNA may be used for preparation of probes for microarray hybridization or may be amplified in PCR reaction (RT-PCR).

Marker, (e.g., transcript) analysis may be by in situ hybridization using a labeled nucleic acid probe. The in situ hybridization is well known in the art.

Depending on the format, the specific nucleic acid of interest is identified in the sample directly using amplification or by hybridization to a labeled (radioactively or fluorescently) nucleic acid probe. The identified amplified product is then detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

### Capture of Markers

Biomarkers are preferably captured with capture reagents immobilized to a solid support, such as any biochip described herein, a multiwell microtiter plate or a resin. The biomarkers of this invention may be captured on protein biochips or microarrays.

Microarrays useful in the methods of the invention for measuring tissue-specific gene expression comprise, for example, the biomarker or anti-sense biomarker polynucleotides, for example, a combination of biomarker and/or anti-sense biomarker polynucleotides from one or more trends. Alternately, the micoarrays comprise at least 4 polynucleotides from Table 9 selected by their differential expression between cancerous and control samples. The invention further contemplates a method of diagnosing a cancer comprising contacting a cell sample nucleic acid with a microarray described herein under conditions suitable for hybridization; providing hybridization conditions suitable for hybrid formation between said cell sample nucleic acid and a polynucleotide of said microarray; detecting said hybridization; and diagnosing a cancer based on the results of detecting said hybridization.

Alternately, biomarkers may be captured on an antibody microarray. The antibody microarray comprises anti-biomarker antibodies, for example, a combination of anti-biomarker antibodies from one or more trends. Alternately, the micoarrays comprise at least 4 antibodies that are anti-biomarker antibodies of gene products from Table 9 selected by their differential expression between cancerous and control cells. The invention further contemplates a method of diagnosing a cancer or wound healing comprising contacting a bodily fluid sample with the antibody microarray described herein, and detecting hybridization between the antibodies present on the array and at least one polypeptide present in the bodily fluid, the results of said detection enabling a diagnosis or a prognosis of a cancer.

In general, a sample containing the biomarkers, such a cell lyste, is placed on the active surface of a biochip for a sufficient time to allow binding. Then, unbound molecules are washed from the surface using a suitable eluant, such as phosphate buffered saline. In general, the more stringent the eluant, the more tightly the proteins must be bound to be retained after the wash. The retained protein biomarkers now can be detected by appropriate means.

### Detection and Measurement of Markers

Once captured on a substrate, e.g., biochip or antibody, any suitable method can be used to measure a marker or markers in a sample. For example, markers can be detected and/or measured by a variety of detection methods including for example, gas phase ion spectrometry methods, optical methods, electrochemical methods, atomic force microscopy and radio frequency methods. Using these methods, one or more markers can be detected.

### Microarray Analyses

The term “microarray” refers to an ordered arrangement of hybridizable array elements. The array elements are arranged so that there are preferably at least two or more different array elements, or for example at least 10, 15, 20, 25, 30, 35, 40, 45, 100, 1000, 2000, 3000, 4000 or more. Array elements are available commercially, for example, from Afformetrix, Inc. Array elements may be on, for example, a 1 cm² substrate surface. The hybridization signal from each of the array elements is individually distinguishable. In one embodiment, the array elements comprise polynucleotide probes. In another embodiment, the array elements comprise antibodies.

DNA-based arrays provide a convenient way to explore the expression of a single polymorphic gene or a large number of genes for a variety of applications. The one or more of the markers identified by the invention may be presented in a DNA microarray for the analysis and expression of these genes in various samples and controls. Microarray chips are well known to those of skill in the art (see, e.g., U.S. Pat. Nos. 6,308,170; 6,183,698; 6,306,643; 6,297,018; 6,287,850; 6,291,183, each incorporated herein by reference). These are exemplary patents that disclose nucleic acid microarrays and those of skill in the art are aware of numerous other methods and compositions for producing microarrays.

Protein and antibody microarrays are well known in the art (see, for example: Ekins R. P., J Pharm Biomed Anal 1989. 7: 155; Ekins R. P. and Chu F. W., Clin Chem 1991. 37: 1955; Ekins R. P. and Chu F. W, Trends in Biotechnology, 1999, 17, 217-218). Antibody microarrays directed against a combination of the diagnostic markers disclosed herein will be very useful for the diagnosis of cancer markers in bodily fluids.

A plurality of polynucleotides identified according to the methods of the invention are useful as biomarkers for diagnosis, prognosis and screening assays described herein. The polynucleotides may be about 9 nucleotides; alternately about 12, 15, 17, 20 nucleotides or longer, depending on the specific use. One of skill in the art would know what length polynucleotide would be appropriate for a particular purpose. Such a plurality of polynucleotides can be employed for the diagnosis and treatment of neoplastic disorder.

The plurality of polynucleotides and/or their anti-sense sequences are useful as hybridizable array elements in a microarray for monitoring the expression of a plurality of biomarker polynucleotides. The microarray comprises a substrate and the hybridizable array elements. The microarray is used, for example, in the diagnosis and treatment of a cancer.

In one aspect, the invention provides a microarray that is a low density array with 384 qPCR reactions to detect biomarkers of the invention in an RNA sample. Premade qPCR reactions for the human discordant genes and standard gene 18s were printed on a low density array (Applied Biosystems). The reactions were printed in replicas

Immunoassay

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

An immunoassay is an assay that uses an antibody to specifically bind an antigen (e.g., a marker). The immunoassay is characterized by the use of specific binding properties of a particular antibody to isolate, biomarker, and/or quantify the antigen. The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a marker from specific species such as rat, mouse, or human can be selected to obtain only those polyclonal antibodies that are specifically immunoreactive with that marker and not with other proteins, except for polymorphic variants and alleles of the marker. This selection may be achieved by subtracting out antibodies that cross-react with the marker molecules from other species.

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Typically a specific or selective reaction will be at least twice background signal or noise and more typically more than 10 to 100 times background.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe D substrate or ProteinChip® array described above. The sample is preferably a biological fluid sample taken from a subject. Examples of biological fluid samples include blood, serum, plasma, nipple aspirate, urine, tears, saliva etc. In a preferred embodiment, the biological fluid comprises blood serum. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Methods for measuring the amount of, or presence of, antibody-marker complex include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro. In a preferred example, the biomarkers are used to differentiate between the different stages of tumor progression, thus aiding in determining appropriate treatment and extent of metastasis of the tumor.

The term “probe” refers to a polynucleotide sequence capable of hybridizing with a biomarker sequence to form a polynucleotide probe/biomarker complex. A “biomarker polynucleotide” refers to a chain of nucleotides to which a polynucleotide probe can hybridize by base pairing. In some instances, the sequences will be complementary (no mismatches) when aligned. In other instances, there may be up to a 10% mismatch. Alternatively, the term “probe” may refer to a polypeptide probe that can hybridize to an antibody.

A “plurality” refers preferably to a group of at least 3 or more members, more preferably to a group of at least about 10, 50, 100, and at least about 1,000, members. The maximum number of members is unlimited, but is at least about 100,000 members.

The term “gene” or “genes” refers to a polynucleotide sequence(s) of a gene, which may be the partial or complete sequence of the gene and may comprise regulatory region(s), untranslated region(s), or coding regions.

The polynucleotide or antibody microarray can be used for large-scale genetic or gene expression analysis of a large number of biomarker polynucleotides or polypeptides respectively. The microarray can also be used in the diagnosis of diseases and in the monitoring of treatments. Further, the microarray can be employed to investigate an individual's predisposition to a disease. Furthermore, the microarray can be employed to investigate cellular responses to infection, drug treatment, and the like.

When the composition of the invention is employed as hybridizable array elements in a microarray, the array elements are organized in an ordered fashion so that each element is present at a distinguishable, and preferably specified, location on the substrate. In the preferred embodiments, because the array elements are at specified locations on the substrate, the hybridization patterns and intensities (which together create a unique expression profile) can be interpreted in terms of expression pattern of particular genes and can be correlated with a particular disease or condition or treatment.

The composition comprising a plurality of polynucleotide probes can also be used to purify a subpopulation of mRNAs, cDNAs, genomic fragments and the like, in a sample. Typically, samples will include biomarker polynucleotides of interest and other nucleic acids which may enhance the hybridization background; therefore, it may be advantageous to remove these nucleic acids from the sample. One method for removing the additional nucleic acids is by hybridizing the sample containing biomarker polynucleotides with immobilized polynucleotide probes under hybridizing conditions. Those nucleic acids that do not hybridize to the polynucleotide probes are removed and may be subjected to analysis or discarded. At a later point, the immobilized biomarker polynucleotide probes can be released in the form of purified biomarker polynucleotides.

### Microarrays

### Microarray Expression Profiles—Expression Profiling

An expression profile can be used to detect changes in the expression of genes implicated in disease. Changes in expression include, up and/or down regulation of a gene.

The expression profile includes a plurality of detectable complexes. Each complex is formed by hybridization of one or more. polynucleotides of the invention to one or more complementary biomarker polynucleotides. At least one of the polynucleotides of the invention, and preferably a plurality thereof, is hybridized to a complementary biomarker polynucleotide forming at least one, and preferably a plurality, of complexes. A complex is detected by incorporating at least one labeling moiety in the complex as described above. The expression profiles provide “snapshots” that can show unique expression patterns that are characteristic of the presence or absence of a disease or condition.

After performing hybridization experiments and interpreting detected signals from a microarray, particular probes can be identified and selected based on their expression patterns. Such probe sequences can be used to clone a full-length sequence for the gene or to produce a polypeptide.

The composition comprising a plurality of probes can be used as hybridizable elements in a microarray. Such a microarray can be employed in several applications including diagnostics, prognostics and treatment regimens, drug discovery and development, toxicological and carcinogenicity studies, forensics, pharmacogenomics, and the like.

The invention provides for microarrays for measuring gene expression characteristic of a cancer of a tissue, comprising at least 4 polypeptide encoding polynucleotides or at least 4 antibodies which bind specifically to the polypeptides encoded by these polynucleotides, as listed in Table 2 and according to the following:

A microarray for measuring gene expression characteristic of renal cancer comprising markers listed in Table 2 sheet 1; A microarray for measuring gene expression characteristic of uterine cancer comprising markers listed in Table 2 sheet 2; A microarray for measuring gene expression characteristic of kidney cancer comprising markers listed in Table 2 sheet 3; A microarray for measuring gene expression characteristic of bladder cancer comprising markers listed in Table 2 sheet 4; A microarray for measuring gene expression characteristic of lung cancer comprising markers listed in Table 2 sheet 5; A microarray for measuring gene expression characteristic of brain cancer comprising markers listed in Table 2 sheet 6; A microarray for measuring gene expression characteristic of colon cancer comprising markers listed in Table 2 sheet 7; A microarray for measuring gene expression characteristic of intestinal cancer comprising markers listed in Table 2 sheet 8; A microarray for measuring gene expression characteristic of stomach cancer comprising markers listed in Table 2, sheet 9; A microarray for measuring gene expression characteristic of renal cancer comprising markers listed in Table 2 sheet 10; A microarray for measuring gene expression characteristic of pancreatic cancer comprising markers listed in Table 2 sheet 11; and A microarray for measuring gene expression characteristic of spleen cancer comprising markers listed in Table 2 sheet 12.

The nucleic acid probes can be genomic DNA or cDNA or mRNA, or any RNA-like or DNA-like material, such as peptide nucleic acids, branched DNAs, and the like. The probes can be sense or antisense polynucleotide probes. Where biomarker polynucleotides are double-stranded, the probes may be either sense or antisense strands. Where the biomarker polynucleotides are single-stranded, the probes are complementary single strands.

In one embodiment, the probes are cDNAs. The size of the DNA sequence of interest may vary and is preferably from 100 to 10,000 nucleotides, more preferably from 150 to 3,500 nucleotides. The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980). Alternatively, the probes can be generated, in whole or in part, enzymatically. Nucleotide analogs can be incorporated into the probes by methods well known in the art. The only requirement is that the incorporated nucleotide analog must serve to base pair with biomarker polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine. Additionally, the probes can include nucleotides that have been derivatized chemically or enzymatically. Typical chemical modifications include derivatization with acyl, alkyl, aryl or amino groups. The polynucleotide probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent. Complementary DNA (cDNA) can be arranged and then immobilized on a substrate. The probes can be immobilized by covalent means such as by chemical bonding procedures or UV. In one such method, a cDNA is bound to a glass surface which has been modified to contain epoxide or aldehyde groups. In another case, a cDNA probe is placed on a polylysine coated surface and then UV cross-linked (Shalon et al., PCT publication WO95/35505, herein incorporated by reference). In yet another method, a DNA is actively transported from a solution to a given position on a substrate by electrical means (Heller et al., U.S. Pat. No. 5,605,662). Alternatively, individual DNA clones can be gridded on a filter. Cells are lysed, proteins and cellular components degraded, and the DNA coupled to the filter by UV cross-linking.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.)

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Alternatively, as mentioned above, antibody microarrays can be produced. The production of such microarrays is essentially as described in Schweitzer & Kingsmore, “Measuring proteins on microarrays”, Curr Opin Biotechnol 2002; 13(1): 14-9; Avseenko et al., “Immobilization of proteins in immunochemical microarrays fabricated by electrospray deposition”, Anal Chem 2001 15; 73(24): 6047-52; Huang, “Detection of multiple proteins in an antibody-based protein microarray system, Immunol Methods 2001 1; 255 (1-2): 1-13. In general, protein microarrays may be produced essentially as described in Schena et al., Parallel human genome analysis: Microarray-based expression monitoring of 1000 genes. Proc. Natl. Sci. USA (1996) 93, 10614-10619; U.S. Pat. Nos. 6,291,170 and 5,807,522 (see above); U.S. Pat. No. 6,037,186 (Stimpson, inventor) “Parallel production of high density arrays”; PCT publications WO 99/13313 (Genovations Inc (US), applicant) “Method of making high density arrays”; WO 02/05945 (Max-Delbruck-center for molecular medicine (Germany), applicant) “Method for producing microarray chips with nucleic acids, proteins or other test substrates”.

Hybridization and Detection in Microarrays

Hybridization causes a denatured probe and a denatured complementary biomarker to form a stable nucleic acid duplex through base pairing. Hybridization methods are well known to those skilled in the art (See, e.g., Ausubel, Short Protocols in Molecular Biology, John Wiley & Sons, New York N.Y., units 2.8-2.11, 3.18-3.19 and 4-6-4.9, 1997). Conditions can be selected for hybridization where an exactly complementary biomarker and probes can hybridize, i.e., each base pair must interact with its complementary base pair. Alternatively, conditions can be selected where a biomarker and probes have mismatches but are still able to hybridize. Suitable conditions can be selected, for example, by varying the concentrations of salt in the prehybridization, hybridization and wash solutions, by varying the hybridization and wash temperatures, or by varying the polarity of the prehybridization, hybridization or wash solutions.

Hybridization can be performed at low stringency with buffers, such as 6×SSPE with 0.005% Triton X-100 at 37° C., which permits hybridization between biomarker and probes that contain some mismatches to form biomarker polynucleotide/probe complexes. Subsequent washes are performed at higher stringency with buffers, such as 0.5×SSPE with 0.005% Triton X-100 at 50° C., to retain hybridization of only those biomarker/probe complexes that contain exactly complementary sequences. Alternatively, hybridization can be performed with buffers, such as 5×SSC/0.2% SDS at 60° C. and washes are performed in 2×SSC/0.2% SDS and then in 0.1×SSC. Background signals can be reduced by the use of detergent, such as sodium dodecyl sulfate, Sarcosyl or Triton X-100, or a blocking agent, such as salmon sperm DNA.

After hybridization, the microarray is washed to remove nonhybridized nucleic acids, and complex formation between the hybridizable array elements and the biomarker polynucleotides is detected. Methods for detecting complex formation are well known to those skilled in the art. In a preferred embodiment, the biomarker polynucleotides are labeled with a fluorescent label, and measurement of levels and patterns of fluorescence indicative of complex formation is accomplished by fluorescence microscopy, preferably confocal fluorescence microscopy. An argon ion laser excites the fluorescent label, emissions are directed to a photomultiplier, and the amount of emitted light is detected and quantitated. The detected signal should be proportional to the amount of probe/biomarker polynucleotide complex at each position of the microarray. The fluorescence microscope can be associated with a computer-driven scanner device to generate a quantitative two-dimensional image of hybridization intensity. The scanned image is examined to determine the * abundance/expression level of each hybridized biomarker polynucleotide.

Typically, microarray fluorescence intensities can be normalized to take into account variations in hybridization intensities when more than one microarray is used under similar test conditions. In a preferred embodiment, individual probe/biomarker hybridization intensities are normalized using the intensities derived from internal normalization controls contained on each microarray.

Protein or antibody microarray hybridization is carried out essentially as described in Ekins et al. J Pharm Biomed Anal 1989. 7: 155; Ekins and Chu, Clin Chem 1991. 37: 1955; Ekins and Chu, Trends in Biotechnology, 1999, 17, 217-218; MacBeath and Schreiber, Science 2000; 289(5485): p. 1760-1763.

### Sample Preparation for Genetic Analysis

To conduct sample analysis, a sample containing biomarker polynucleotides or polypeptides is provided. The samples can be any sample containing biomarker polynucleotides or polypeptides and obtained from any bodily fluid blood, sperm, urine, saliva, phlegm, gastric juices, etc. as described herein), cultured cells, biopsies, or other tissue preparations. The samples being analyzed using the microarrays will likely be samples from individuals suspected of suffering from a given cancer. In one embodiment, the microarrays used are those that contain tumor markers specific for that cancer or antibodies against those markers.

DNA or RNA can be isolated from the sample according to any of a number of methods well known to those of skill in the art. For example, methods of purification of nucleic acids are described in Tijssen Laboratory Techniques in Biochemistry and Molecular Biology: Hybridization With Nucleic Acid Probes, Part I. Theory and Nucleic Acid Preparation, Elsevier, New York N.Y. 1993. In one case, total RNA is isolated using the TRIZOL reagent (Life Technologies, Gaithersburg Md.), and mRNA is isolated using oligo d(T) column chromatography or glass beads. Alternatively, when biomarker polynucleotides are derived from an mRNA, the biomarker polynucleotides can be a cDNA reverse-transcribed from an mRNA, an RNA transcribed from that cDNA, a DNA amplified from that cDNA, an RNA transcribed from the amplified DNA, and the like. When the biomarker polynucleotide is derived from DNA, the biomarker polynucleotide can be DNA amplified from DNA or RNA reverse transcribed from DNA. In yet another alternative, the biomarkers are biomarker polynucleotides prepared by more than one method.

When biomarker polynucleotides are amplified, it is desirable to amplify the nucleic acid sample and maintain the relative abundances of the original sample, including low abundance transcripts. Total mRNA can be amplified by reverse transcription using a reverse transcriptase and a primer consisting of oligo d(T) and a sequence encoding the phage T7 promoter to provide a single-stranded DNA template. The second DNA strand is polymerized using a DNA polymerase and a RNAse which assists in breaking up the DNA/RNA hybrid. After synthesis of the double-stranded DNA, T7 RNA polymerase can be added, and RNA transcribed from the second DNA strand template (Van Gelder et al. U.S. Pat. No. 5,545,522). RNA can be amplified in vitro, in situ or in vivo (See Eberwine, U.S. Pat. No. 5,514,545).

Controls may be included within the sample to assure that amplification and labeling procedures do not change the true distribution of biomarker polynucleotides in a sample. For this purpose, a sample is spiked with a known amount, of a control biomarker polynucleotide and the composition of probes includes reference probes which specifically hybridize with the control biomarker polynucleotides. After hybridization and processing, the hybridization signals obtained should accurately the amounts of control biomarker polynucleotide added to the sample.

Prior to hybridization, it may be desirable to fragment the nucleic acid biomarker polynucleotides. Fragmentation improves hybridization by minimizing secondary structure and cross-hybridization to other nucleic acid biomarker polynucleotides in the sample or noncomplementary polynucleotide probes. Fragmentation can be performed by mechanical or chemical means.

Antibodies against the relevant cancer marker polypeptides and appropriate for attachment to an antibody microarray can be prepared according to methods known in the art (Coligan et al, Unit 9, Current Protocols in Immunology, Wiley Interscience, 1994; Harlow and Lane, Antibodies: A Laboratory Manual, Cold Spring Harbor Laboratory, New York (1988). Additional information regarding all types of antibodies, including humanized antibodies, human antibodies and antibody fragments can be found in WO 01/05998).

Polypeptides can be prepared for hybridization to an antibody microarray from a sample, such as a bodily fluid sample, according to methods known in the art. It may be desirable to purify the proteins from the sample or alternatively, to remove certain impurities which may be present in the sample and interfere with hybridization. Protein purification is practiced as is known in the art as described in, for example, Marshak et al., “Strategies for Protein Purification and Characterization. A laboratory course manual.” CSHL Press (1996).

The biomarker polynucleotides or polypeptides may be labeled with one or more labeling moieties to allow for detection of hybridized probe/biomarker complexes. The labeling moieties can include compositions that can be detected by spectroscopic, photochemical, biochemical, bioelectronic, immunochemical, electrical, optical or chemical means. The labeling moieties include radioisotopes, such as ³H, ¹⁴C, ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Exemplary dyes include quinoline dyes, triarylmethane dyes, phthaleins, azo dyes, cyanine dyes, and the like. Preferably, fluorescent markers absorb light above about 300 nm, preferably above 400 nm, and usually emit light at wavelengths at least greater than 10 nm above the wavelength of the light absorbed. Preferred fluorescent markers include fluorescein, phycoerythrin, rhodamine, lissamine, and C3 and C5 available from Amersham Pharmacia Biotech (Piscataway N.J.).

Nucleic acid labeling can be carried out during an amplification reaction, such as polymerase chain reactions and in vitro transcription reactions, or by nick translation or 5′ or 3′-end-labeling reactions. When the label may be incorporated after or without an amplification step, the label is incorporated by using terminal transferase or by phosphorylating the 5′ end of the biomarker polynucleotide using, e.g., a kinase and then incubating overnight with a labeled oligonucleotide in the presence of T4 RNA ligase. Alternatively, the labeling moiety can be incorporated after hybridization once a probe/biomarker complex has formed.

Polypeptide labeling can be conducted using a variety of techniques well known in the art, and the choice of the technique(s) can be tailored to the polypeptide in question according to criteria known to one of skill in the art. Specifically, polypeptides can be fluorescently labeled with compounds such as FITC or rhodamin, essentially as described in Harlow and Lane, Antibodies: A Laboratory Manual, Cold Spring Harbor Laboratory, New York (1988), in particular pages 353-356, or with other fluorescent compounds such as nile red or 2-methoxy-2,4-diphenyl-3(2H)fur-anone (Daban: Electrophoresis 2001; 22(5): 874-80). Polypeptides can also be labeled with a detectable protein such as GFP (detection based on fluorescence) or the vitamin biotin (detection with streptavidin). Polypeptides can also be radioactively labeled with the isotope S³⁵. Additional methods are widely known in the art.

### Use of Gene Sequences for Diagnostic Purposes

In certain embodiments, the tissue-specific tumor markers identified herein may be used for the diagnosis of advanced stages of cancer in the given tissue for which the markers are specific. The polynucleotide sequences encoding the tissue specific tumor marker or the polypeptide encoded thereby, where appropriate, may be used in in-situ hybridization or RT-PCR assays of fluids or tissues from biopsies to detect abnormal gene expression. Such methods may be qualitative or quantitative in nature and may include Southern or Northern analysis, dot blot or other membrane-based technologies; PCR technologies; chip based technologies (for nucleic acid detection) and dip stick, pin, ELISA and protein-chip technologies (for the detection of polypeptides). All of these techniques are well known in the art and are the basis of many commercially available diagnostic kits.

In addition, such assays may be useful in evaluating the efficacy of a particular therapeutic treatment regime in animal studies, in clinical trials, or in monitoring the treatment of an individual patient. Such monitoring may generally employ a combination of body fluids or cell extracts taken from normal subjects, either animal or human, under conditions suitable for hybridization or amplification. Standard hybridization may be quantified by comparing the values obtained for normal subjects with a dilution series of a tissue-specific tumor marker gene product run in the same experiment where a known amount of purified gene product is used. Standard values obtained from normal samples may be compared with values obtained from samples from cachectic subjects affected by abnormal gene expression in tumor cells. Deviation between standard and subject values establishes the presence of disease.

Generally, the tissue-specific tumor markers are chosen based on the specificity of their expression in tumors as well as on the high correlation of the reactivity of corresponding antibodies with tumor specimens in ELISA and tissue arrays may be used for development of serological screening procedure. For example, in the context of prostate-specific tumor markers, a large scale analysis of serum and sperm samples obtained from normal donors of different age (before and after 60), patients with different grades and types of prostate carcinoma, androgen dependent and androgen independent, with local, recurrent and metastatic disease, patients with, tumors of other than prostate origin, as well as patients with noncancerous diseases of prostate may be tested by ELISA on the presence and concentration of the potential candidate polypeptide(s). Then statistical analyses may be performed to evaluate whether the prostate samples express candidate(s) at different expression patterns based on different parameters (histopathological type, Gleason score, tumor size, disease or PSA recurrence).

Once disease is established, a therapeutic agent is administered; and a treatment profile is generated. Such assays may be repeated on a regular basis to evaluate whether the values in the profile progress toward or return to the normal or standard pattern. Successive treatment profiles may be used to show the efficacy of treatment over a period of several days or several-months.

Polymerase Chain Reaction (PCR) as described in, for example, U.S. Pat. Nos. 4,683,195 and 4,965,188, provides additional uses for oligonucleotides specific for the tissue-specific tumor marker genes. Such oligomers are generally chemically synthesized, but they may be generated enzymatically or produced from a recombinant source as described herein above. Oligomers generally comprise two nucleotide sequences, one with sense orientation and one with antisense orientation, employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences. Methods of performing RT-PCR are standard in the art and the method may be carried out using commercially available kits. Other PCR techniques are well known to one of skill in the art, and include, for example, qPCR, real time PCR, reverse transcriptase PCR, PCR done in high density arrays, e.g., open arrays.

Additionally, methods to quantitate the expression of a particular molecule include radiolabeling (Melby et al., J Immunol Methods, 159: 235-244 (1993) or biotinylating (Duplaa et al., Anal Biochem, 229-236 (1993) nucleotides, coamplification of a control nucleic acid, and standard curves onto which the experimental results are interpolated. Quantitation of multiple samples may be speeded up by running the assay in an ELISA-like format where the oligomer of interest is presented in various dilutions and a spectrophotometric or colorimetric response gives rapid quantitation. For example, the presence of abnormal levels or expression patterns of a tissue-specific tumor marker in extracts of biopsied tissues will be indicative of the onset of a cancer. A definitive diagnosis of this type may allow health professionals to begin aggressive treatment and prevent further worsening of the condition. Similarly, further assays can be used to monitor the progress of a patient during treatment.

### Immunodiagnosis and Polypeptide Detection

In certain embodiments, antibodies may be used in characterizing the tissue-specific tumor marker content of healthy and diseased tissues, through techniques such as ELISAs, immunohistochemical detection and Western blotting.

This may provide a screen for the presence or absence of malignancy or as a predictor of future cancer. Once the tissue-specific tumor marker is identified, one of skill in the art may produce antibodies against that marker using techniques well known to those of skill in the art

The use of such antibodies in an ELISA assay is contemplated. For example,: such antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the biological sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for the tumor marker that differs from the first antibody. Appropriate conditions preferably include diluting the sample with diluents such as BSA, bovine gamma globulin (BGG) and phosphate buffered saline (PBS)/Tween. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A preferred washing procedure includes washing with a solution such as PBS/Tween, or borate buffer.

For convenient detection purposes, the second antibody may preferably have an associated enzyme that will generate a color development upon incubating with an appropriate chromogenic substrate. Thus, for example, one will desire to contact: and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and hydrogen peroxide, in the case of peroxidase as the enzyme-label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

Immunoblotting and immunohistochemical techniques using antibodies directed against the tumor markers also are contemplated by the invention. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

Flow cytometry methods also may be used in conjunction with the invention. Methods of performing flow cytometry are discussed in Zhang et al., J Immunology, 157:3980-3987 (1996) and Pepper et al., Leuk. Res., 22(5):439-444 (1998). Generally, the cells, preferably blood cells, are permeabilized to allow the antibody to enter and exit the cell. If the gene in question encodes a cell surface protein, the step of permeabilization is not needed. After permeabilization, the cells are incubated with an antibody. In preferred embodiments, the antibody is a monoclonal antibody. It is more preferred that the monoclonal antibody be labeled with a fluorescent marker. If the antibody is not labeled with a fluorescent marker, a second antibody that is immunoreactive with the first antibody and contains a fluorescent marker. After sufficient washing to ensure that excess or non-bound antibodies are removed, the cells are ready for flow cytometry. If the marker is an enzyme, the reaction monitoring its specific enzymatic activity either in situ or in body fluids may be performed.

Determining the expression pattern of a polypeptide in a sample for the purposes of diagnosis may also be carried out in the form of enzymatic activity testing, when the polypeptide being examined offers such an option.

In addition, whole body image analysis following injection of labeled antibodies against cell surface marker proteins is a diagnostic possibility, as described above; the detected concentrations of such antibodies are indicative of the sites of tumor/metastases growth as well as their number and the tumor size.

### Therapeutic Methods of Using Identified Markers

The genes identified by the invention herein as down-regulated by the loss of a biomarker may prove effective against a given cancer when delivered therapeutically to the cancer cells. Antisense constructs of the genes identified herein as up-regulated as a result of loss of biomarker can be delivered therapeutically to cancer cells. Other therapeutic possibilities include siRNA, RNAi or small molecules or antibodies inhibiting the biomarker protein function and/or expression. The goal of such therapy is to retard the growth rate of the cancer cells. Expression of the sense molecules and their translation products or expression of the antisense mRNA molecules has the effect of inhibiting the growth rate of cancer cells or inducing apoptosis. Sense nucleic acid molecules are preferably delivered in constructs wherein a promoter is operatively linked to the coding sequence at the 5′-end and initiates transcription of the coding sequence. Anti-sense constructs contain a promoter operatively linked to the coding sequence at the 3′-end such that upon initiation of transcription at the promoter an RNA molecule is transcribed which is the complementary strand from the native mRNA molecule of the gene.

Delivery of nucleic acid molecules can be accomplished by many means known in the art. Gene delivery vehicles are available for delivery of polynucleotides to cells, tissue, or to a mammal for expression.

### Antibodies

In one aspect, antibodies can be produced that are specific to one or more of the biomarkers listed in Table 9. The antibodies may be used, for example, to detect the biomarkers in the screening and diagnostic methods according the invention. The antibodies may also be made into an antibody array for use in the methods of the invention.

Various procedures known in the art may be used for the production of antibodies against the biomarkers, or fragments, derivatives, homologs or analogs of the proteins. Antibodies of the invention include, but are not limited to, synthetic antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to an antigen (e.g., one or more complementarity determining regions (CDRs) of an antibody).

For production of the antibody, various host animals can be immunized by injection with, e.g., a native biomarker protein or a synthetic version, or a derivative of the foregoing. Such host animals include, but are not limited to, rabbits, mice, rats, etc. Various adjuvants can be used to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacille Calmette-Guerin (BCG) and Corynebacterium parvum. Although the following refers specifically to a biomarker, any of the methods described herein apply equally to a biomarker, concordantly or discordantly expressed gene family members or subunits thereof.

For preparation of monoclonal antibodies directed towards a biomarker, any technique that provides for the production of antibody molecules by continuous cell lines in culture may be used. Such techniques include, but are not restricted to, the hybridoma technique originally developed by Kohler and Milstein (1975, Nature 256:495-497), the trioma technique (Gustafsson et al., 1991, Hum. Antibodies Hybridomas 2:26-32), the human B-cell hybridoma technique (Kozbor et al., 1983, Immunology Today 4:72), and the EBV hybridoma technique to produce human monoclonal antibodies (Cole et al., 1985, In: Monoclonal Antibodies and Cancer Therapy, Alan R. Liss, Inc., pp. 77-96). In an additional embodiment of the invention, monoclonal antibodies can be produced in germ-free animals utilizing recent technology described in International Patent Application PCT/US90/02545.

According to the present invention, human antibodies may be used and can be obtained by using human hybridomas (Cote et al., 1983, Proc. Natl. Acad. Sci. USA 80:2026-2030) or by transforming human B cells with EBV virus in vitro (Cole et al, 1985, In: Monoclonal Antibodies and Cancer Therapy, Alan R. Liss, Inc., pp. 77-96). In fact, according to the invention, techniques developed for the production of “chimeric antibodies” (Morrison et al., 1984, Proc. Natl. Acad. Sci. USA 81:6851-6855; Neuberger et al., 1984, Nature 312:604-608; Takeda et al, 1985, Nature 314:452-454) by splicing the genes from a mouse antibody molecule specific for a biomarker together with genes from a human antibody molecule of appropriate biological activity can be used; such antibodies are within the scope of this invention.

According to the present invention, techniques described for the production of single chain antibodies (U.S. Pat. No. 4,946,778) can be adapted to produce a biomarker-specific antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for a biomarker proteins. Non-human antibodies can be “humanized” by known methods (e.g., U.S. Pat. No. 5,225,539).

Antibody fragments that contain the idiotypes of a biomarker can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments. Synthetic antibodies, e.g., antibodies produced by chemical synthesis, are useful in the present invention.

In the production of antibodies, screening for the desired antibody can be accomplished by techniques known in the art, e.g., ELISA (enzyme-linked immunosorbent assay). To select antibodies specific to a particular domain of a biomarker or derivatives, homologs, or analogs thereof, one may assay generated hybridomas for a product that binds to the fragment of the a biomarker, that contains such a domain.

An “epitope”, as used herein, is a portion of a polypeptide that is recognized (i.e., specifically bound) by a B-cell and/or T-cell surface antigen receptor. Epitopes may generally be identified using well known techniques, such as those summarized in Paul, Fundamental Immunology, 3rd ed., 243-247 (Raven Press, 1993) and references cited therein. Such techniques include screening polypeptides derived from the native polypeptide for the ability to react with antigen-specific antisera and/or T-cell lines or clones. An epitope of a polypeptide is a portion that reacts with such antisera and/or T-cells at a level that is similar to the reactivity of the full length polypeptide (e.g., in an ELISA and/or T-cell reactivity assay). Such screens may generally be performed using methods well known to those of ordinary skill in the art, such as those described in Harlow and Lane, Antibodies: A Laboratory Manual, Cold Spring Harbor Laboratory, 1988. B-cell and T-cell epitopes may also be predicted via computer analysis. Polypeptides comprising an epitope of a polypeptide that is preferentially expressed in a tumor tissue (with or without additional amino acid sequence) are within the scope of the present invention.

Methods for detecting the expression of a protein biomarker may also include extracting the protein contents of the cells, or extracting fragments of protein from the membranes of the cells, or from the cytosol, for example, by lysis, digestive, separation, fractionation and purification techniques, and separating the proteinaceous contents of the cells (either the crude contents or the purified contents) on a western blot, and then detecting the presence of the protein, or protein fragment by various identification techniques known in the art. For example, the contents separated on a gel may be identified by using suitable molecular weight markers together with a protein identification technique, or using suitable detecting moieties (such as labeled antibodies, labeled lectins, labeled binding agents (agonists, antagonists, substrates, co-factors, ATP, etc.).

Antibodies useful in the techniques of the invention and, for example, specific for the biomarkers listed in Table 9 may be available commercially or made by one of skill in the art. These antibodies are useful in the methods described. For example, one or more of these antibodies, as well as one or more of the antibodies generated to the biomarkers, may be part of an antibody array. Such an antibody array can be used to screen samples from subjects as described herein for diagnostic and screenings purposes. Manufacturer information on candidate antibodies to the discordant genes is available at http://www.linscottsdirectory.com. Based on the database Immunoquery http://www.Immunoquery.com). Each marker has the diagnosis to which it is linked, number of positives found and total number of cases in it was used for diagnosis.

### Diagnosis of Subject and Determination of Renal Status

Any biomarker (e.g., the discordantly expressed transcripts listed in Tables 5-20, and 11) individually, is useful in aiding in the determination of renal status. First, the selected biomarker is measured in a subject sample using the methods described herein, e.g., capture on a nucleic acid microarray followed by detection. Then, the measurement is compared with a diagnostic amount or control that distinguishes renal status, e.g., injured, cancerous or normal renal status. The diagnostic amount will reflect the information herein that a particular biomarker is up-regulated or down-regulated in a cancer status compared with a non-cancer status. As is well understood in the art, the particular diagnostic amount used can be adjusted to increase sensitivity or specificity of the diagnostic assay depending on the preference of the diagnostician. The test amount as compared with the diagnostic amount thus indicates renal status.

In one embodiment, biomarkers include for example, discordant genes (e.g., down-regulated in RRR and up-regulated in RRC. Discordant biomarkers for RRR, include, for example any one or more of, or a combination of, IGFBP1, IGFBP3, CTGF, AKT, FRAP, MYC, NF-κB, HK1 and SIRT7. In one embodiment, biomarker for RRR comprise, for example, IGFBP1 and IGFBP3; IGFBP1 and CTGF; IGFBP1 and AKT; IGFBP1 and FRAP; IGFBP1 and MYC; IGFBP1 and NF-κB; IGFBP1 and HK1; IGFBP1 and SIRT7; IGFBP1, IGFBP3 and CTGF; IGFBP1, IGFBP3 and AKT; CTGF, AKT, FRAP, MYC, NF-κB, HK1 and SIRT7 FRAP; IGFBP1, IGFBP3 and MYC; IGFBP1, IGFBP3 and NF-κB; IGFBP1, IGFBP3 and HK1; IGFBP1, IGFBP3 and SIRT7; and other combinations. In one embodiment, a biomarker of RRC comprises HK1, which is upregulated in RRC and down-regulated in RRR.

While individual biomarkers are useful diagnostic markers, it has been found that a combination of biomarkers provides greater predictive value than single markers alone. Specifically, the detection of a plurality of markers in a sample increases the percentage of true positive and true negative diagnoses and would decrease the percentage of false positive or false negative diagnoses. Thus, preferred methods of the present invention comprise the measurement of more than one biomarker. For example, measuring two or more markers from one or more clusters of markers.

In some embodiments, the mere presence or absence of a marker, without quantifying the amount of marker, is useful and can be correlated with a probable diagnosis of renal cancer. For example, Table 8 lists the times specific biomarkers are expressed in RRR and RCC cells. Thus, the detection of a particular biomarker is indicative of that cell's status and a detected presence or absence, respectively, of these markers in a subject being tested indicates that the subject has a higher probability of having renal cancer.

In other embodiments, the measurement of markers can involve quantifying the markers to correlate the detection of markers with a probable diagnosis of renal cancer. Thus, if the amount of the markers detected in a subject being tested is different compared to a control amount (i.e., higher or lower than the control, depending on the marker), then the subject being tested has a higher probability of having renal cancer.

The correlation may take into account the amount of the marker or markers in the sample compared to a control amount of the marker or markers (up or down regulation of the marker or markers) (e.g., in normal subjects in whom human cancer is undetectable). A control can be, e.g., the average or median amount of marker present in comparable samples of normal subjects in whom human cancer is undetectable. The control amount is measured under the same or substantially similar experimental conditions as in measuring the test amount. The correlation may take into account the presence or absence of the markers in a test sample and the frequency of detection of the same markers in a control. The correlation may take into account both of such factors to facilitate determination of renal status.

In certain embodiments of the methods of qualifying renal status, the methods further comprise managing subject treatment based on the status. As aforesaid, such management describes the actions of the physician or clinician subsequent to determining renal status. For example, if the result of the methods of the present invention is inconclusive or there is reason that confirmation of status is necessary, the physician may order more tests. Alternatively, if the status indicates that surgery is appropriate, the physician may schedule the patient for surgery. In other instances, the patient may receive chemotherapy or radiation treatments, either in lieu of, or in addition to, surgery. Likewise, if the result is negative, e.g., the status indicates late stage renal cancer or if the status is otherwise acute, no further action may be warranted. Furthermore, if the results show that treatment has been successful, no further management may be necessary.

The invention also provides for such methods where the biomarkers (or specific combination of biomarkers) are measured again after subject management. In these cases, the methods are used to monitor the status of the cancer, e.g., response to cancer treatment, remission of the disease or progression of the disease. Because of the ease of use of the methods and the lack of invasiveness of the methods, the methods can be repeated after each treatment the patient receives. This allows the physician to follow the effectiveness of the course of treatment. If the results show that the treatment is not effective, the course of treatment can be altered accordingly. This enables the physician to be flexible in the treatment options.

In another example, the methods for detecting markers can be used to assay for and to identify compounds that modulate expression of these markers in vivo or in vitro.

The methods of the present invention have other applications as well. For example, the markers can be used to screen for compounds that modulate the expression of the markers in vitro or in vivo, which compounds in turn may be useful in treating or preventing renal cancer in patients. In another example, the markers can be used to monitor the response to treatments for renal cancer. In yet another example, the markers can be used in heredity studies to determine if the subject is at risk for developing renal cancer. For instance, certain markers may be genetically linked. This can be determined by, e.g., analyzing samples from a population of renal cancer patients whose families have a history of renal cancer. The results can then be compared with data obtained from, e.g., renal cancer patients whose families do not have a history of renal cancer. The markers that are genetically linked may be used as a tool to determine if a subject whose family has a history of renal cancer is pre-disposed to having renal cancer.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the presence or absence in a test subject of any the biomarkers of this invention is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

The term diagnosis as used herein generally comprises any kind of assessment of the presence of absence of a medically relevant condition. Diagnosis thus comprises processes such as screening for the predisposition for a medically relevant condition, screening for the precursor of a medically relevant condition, screening for a medically relevant condition, clinical or pathological diagnosis of a medically relevant condition, etc. Diagnosis of medically relevant conditions as used herein may comprise examination of any condition, that is detectable on a cytological, histological, biochemical or molecular biological level, that may be useful in respect to the human health and/or body. Such examinations may comprise e.g., medical diagnostic methods and research studies in life sciences. In one embodiment of the invention, the method is used for diagnosis of medically relevant conditions such as e.g., diseases. Such diseases may for example comprise disorders characterized by proliferation of cells or tissues.

In one embodiment, the diagnosis pertains to diagnosis of cancers and their precursory stages, to monitoring of the disease course in cancers, to assessment of prognosis in cancers and to detection of disseminated tumor cells, e.g., in the course of minimal residual disease diagnosis. The methods according to the present invention may for example be used in the course of clinical or pathological diagnosis of cancers and their precursory stages or in routine screening tests as performed for particular cancers such as for example for examination of swabs e.g. in screening tests for renal cancer.

One aspect of this normalization includes comparing the results of a determination of one or more of the parameters disclosed herein and determining one or more of the cellular expression pattern of a biomarker.

Correlating may include making an assessment that a particular result is not accurate. Correlating may also include predicting whether a certain marker is a meaningful in the context of diagnosis, prognosis, and/or monitoring of treatment. Correlating may be done by mathematical formulae, computer program, or a person. As disclosed herein, certain markers are predictive of disease state or progression of disease state. Correlating or normalization, especially in the context of a diagnosis, may also include or take into consideration, such factors as, the total number of cells present in the sample, of the presence or absence of a particular cell type or types in a sample, the presence or absence of an organism or of cells of an organism in a sample, the number of cells of a particular cell type or organism present in the sample, the proliferative characteristics of cells present in the sample, or the differentiation pattern of the cells present in the sample.

In certain embodiments normalization may also comprise demonstrating the adequacy of the test, wherein as the case may be inadequate test results may be discarded or classified as invalid. Therefore normalization as used in the context of the present invention may comprise qualitative or semi-quantitative methods for normalization. In certain embodiments, semi-quantitative normalization may comprise determining a threshold value for a normalization marker.

### Therapeutic Candidates and Methods of Treatment

The methods of the present invention have other applications as well. For example, the biomarkers can be used to screen for compounds that modulate the expression of the biomarkers in vitro or in vivo, which compounds in turn may be useful in treating or preventing renal cancer in patients. In another example, the biomarkers can be used to monitor the response to treatments for renal cancer. In yet another example, the biomarkers can be used in heredity studies to determine if the subject is at risk for developing renal cancer.

Thus, for example, the kits of this invention could include a solid substrate, such as a nucleic acid biochip and a buffer for washing the substrate, as well as instructions providing a protocol to measure the biomarkers of this invention on the chip and to use these measurements to diagnose renal cancer.

Based on the results of the analysis, identified among the concordant and discordant genes and other genes in their pathways, were compounds that could be used as gene-drug targets. The pharmaceutical composition identified through the screening methods of the invention may be given in combination. Useful combinations of therapeutics will offer one or more of the following improvements over a single composition therapeutic: improve the efficacy of one or more of the therapeutics in the composition, lower the dosage of one or more of the therapeutics in the composition, decrease the time of action of one or more of the therapeutics in the composition, decrease the toxicity of one or more of the therapeutics in the composition. Therapeutics that may be given in combination include the therapeutics identified by, linked or generated by the software program and database as PharmaProjects as well as the therapeutics identified in the screening methods of the invention. The therapeutics can be used to treat, for example, RCC, acute renal failure, RRR, organ transplantation, organ shipment, wound healing, other tumors and organ failure.

Compounds suitable for therapeutic testing may be screened initially, for example, by identifying compounds which interact with one or more biomarkers listed in identified herein or compounds that are known to interact with a biomarker.

In a related embodiment, the ability of a test compound to alter the expression profile of one or more of the biomarkers of this invention may be measured. One of skill in the art will recognize that the techniques used to measure the expression profile of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the expression profile of a given biomarker may be determined by measuring the rates of catalysis in the presence or absence of the test compounds. The ability of a test compound to interfere with a non-enzymatic (e.g., structural) function or expression profile of one of the biomarkers of this invention may also be measured. For example, the self-assembly of a multi-protein complex which includes one of the biomarkers of this invention may be monitored by spectroscopy in the presence or absence of a test compound. Alternatively, if the biomarker is a non-enzymatic enhancer of transcription, test compounds which interfere with the ability of the biomarker to enhance transcription may be identified by measuring the expression patterns of biomarker-dependent transcription in vivo or in vitro in the presence and absence of the test compound. Test compounds capable of modulating the expression profile of any of the biomarkers of this invention may be administered to patients who are suffering from or are at risk of developing renal carcinoma or other cancer. For example, the administration of a test compound which alters the expression profile of a discordantly expressed marker may decrease the risk of renal cancer in a patient.

In yet another embodiment, the invention provides a method for treating or reducing the progression or likelihood of a disease, e.g., renal carcinoma. For example, after one or more markers have been identified which are predictive of the state of a sample, e.g., whether the sample is benign, is in the initiation phase, extension phase, maintenance phase, or is carcinoma, combinatorial libraries may be screened for compounds which alter the expression profile of the markers toward a normal or health, or regeneration and/or repair profile. Methods of screening chemical libraries for such compounds are well-known in art. See, e.g., Lopez-Otin et al. (2002). At the clinical level, screening a test compound includes obtaining samples from test subjects before and after the subjects have been exposed to a test compound. The expression patterns in the samples of one or more of the biomarkers of this invention may be measured and analyzed to determine whether the expression patterns of the biomarkers change after exposure to a test compound. The samples may be analyzed by mass spectrometry, as described herein, or the samples may be analyzed by any appropriate means known to one of skill in the art. For example, the expression patterns of one or more of the biomarkers of this invention may be measured directly by Western blot using radio- or fluorescently-labeled antibodies which specifically bind to the biomarkers. Alternatively, changes in the expression patterns of mRNA encoding the one or more biomarkers may be measured and correlated with the administration of a given test compound to a subject. In a further embodiment, the changes in the expression pattern of expression of one or more of the biomarkers may be measured using in vitro methods and materials. For example, human tissue cultured cells which express, or are capable of expressing, one or more of the biomarkers of this invention may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with renal cancer, test compounds will be screened for their ability to slow or stop the progression of the disease. For protein biochips, test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of this invention, in which case the proteins may be detected by monitoring the digestion of one or more biomarkers in a standard assay, e.g., by gel electrophoresis of the proteins.

The invention provides methods for identifying modulators, i.e., candidate or test compounds or agents (e.g. peptides, small molecules or other drugs) that have a stimulatory or inhibitory effect on the pathway(s) affected by the agent and have anti-proliferative properties. Such compounds may include, but are not limited to, peptides made of D- and/or L-configuration amino acids (in, for example, the form of random peptide libraries; (see e.g., Lam, et al., Nature, 354:82-4 (1991)), phosphopeptides (in, for example, the form of random or partially degenerate, directed phosphopeptide libraries; see, e.g., Songyang, et al., Cell, 72:767-78 (1993)), antibodies, and small organic or inorganic molecules. Compounds identified may be useful, for example, in modulating the activity of a biomarker pathway biomarker gene proteins, (e.g., cellular expression pattern of RXR-alpha).

In one embodiment, the invention provides libraries of test compounds. The test compounds of the present invention can be obtained using any of the numerous approaches in combinatorial library methods known in the art, including: biological libraries, spatially addressable parallel solid phase or solution phase libraries; synthetic library methods requiring deconvolution; the one-bead one-compound library method; and synthetic library methods using affinity chromatography selection. The biological library approach is exemplified by peptide libraries, while the other four approaches are applicable to peptide, non-peptide oligomer or small molecule libraries of compounds (Lam, K. S. (1997) “Application of combinatorial library methods in cancer research and drug discovery.” Anticancer Drug Des. 12:145).

Methods for the synthesis of molecular libraries can be found in the art, for example, in (i) De Witt, S. H. et al. (1993) “Diversomers: an approach to nonpeptide, nonoligomeric chemical diversity.” PNAS 90:6909, (ii) Erb, E. et al. (1994) “Recursive deconvolution of combinatorial chemical libraries.” PNAS 91:11422, (iii) Zuckermann, R. N. et al. (1994) “Discovery of nanomolar ligands for 7-transmembrane G-protein-coupled receptors from a diverse N-(substituted)glycine peptide library.” J. Med Chem. 37: 2678 and (iv) Cho, C. Y. et al. (1993) “An unnatural biopolymer.” Science 261:1303. Libraries of compounds may be presented in i) solution (e.g. Houghten, R. A. (1992) “The use of synthetic peptide combinatorial libraries for the identification of bioactive peptides.” BioTechniques 13:412) ii) on beads (Lam, K. S. (1991) “A new type of synthetic peptide library for identifying ligand-binding activity.” Nature 354:82), iii) chips (Fodor, S. P. (1993) “Multiplexed biochemical assays with biological chips.” Nature 364:555), iv) bacteria (U.S. Pat. No. 5,223,409), v) spores (U.S. Pat. Nos. 5,571,698, 5,403,484, and 5,223,409), vi) plasmids (Cull, M. G. et al. (1992) “Screening for receptor ligands using large libraries of peptides linked to the C terminus of the lac repressor.” PNAS 89:1865) or vii) phage (Scott; J. K. and Smith, G. P. (1990) “Searching for peptide ligands with an epitope library.” Science 249: 386)

The practice of the present invention employs, unless otherwise indicated, conventional molecular biology, microbiology, and recombinant DNA techniques within the skill of the art. Such techniques are explained fully in the literature. See, e.g., Maniatis, Fritsch & Sambrook, In Molecular Cloning: A Laboratory Manual (1982); DNA Cloning: A Practical Approach, Volumes I and II, D. N. Glover, ed., (1985); Oligonucleotide Synthesis, M. J. Gait, ed., (1984); Ausubel, et al., (eds.), Current Protocols In Molecular Biology, John Wiley & Sons, New York, N.Y. (1993); Nucleic Acid Hybridization, B. D. Hames & S. J. Higgins, eds., (1985); Transcription and Translation, B. D. Hames & S. I. Higgins, eds., (1984); Animal Cell Culture, R. I. Freshney, ed. (1986); and B. Perbal, A Practical Guide to Molecular Cloning (1984).

As used herein, “comparing” in relation to “cellular expression pattern of a biomarker refers to making an assessment of the how the cellular expression pattern of a sample relates to the cellular expression pattern of the standard. For example, assessing whether the cellular expression pattern of the sample is different from the cellular expression pattern of the standard cellular expression pattern, for example of a reference cell as described herein.

In a particular embodiment, the present invention provides a method for treating a disease or disorder characterized by aberrant cellular expression pattern of a biomarker comprising administering to a subject having such disease or disorder a composition comprising a molecule that alters the subcellular expression pattern of a biomarker and a pharmaceutically acceptable carrier.

Once obtained, the results of any assay herein may be reported to the subject or a health care professional, e.g., reporting the cellular expression pattern of a biomarker. The report to the subject may also be accompanied by a diagnosis and recommendations for treatment.

Following diagnosis or assessment of likelihood of an efficacious result, the treatment may include surgery, focal therapy (mucosectomy, argon plasma coagulator, cryotherapy), selenium fortification, chemoradiation therapy, chemotherapy, radiotherapy, including but not limited to, tamoxifen, trastuzamab (herceptin), raloxifene, doxorubicin, fluorouracil/5-fu, pamidronate disodium, anastrozole, exemestane, cyclophos-phamide, epirubicin, letrozole, toremifene, fulvestrant, fluoxymester-one, trastuzumab, methotrexate, megastrol acetate, docetaxel, paclitaxel, testolactone, aziridine, vinblastine, capecitabine, goselerin acetate, zoledronic acid, taxol. The appropriate treatment for a particular subject may be determined by one of skill in the art.

The identification of those patients who are in need of prophylactic treatment for cancer is well within the ability and knowledge of one skilled in the art. Certain of the methods for identification of patients which are at risk of developing cancer which can be treated by the subject method are appreciated in the medical arts, such as family history, travel history and expected travel plans, the presence of risk factors associated with the development of that disease state in the subject patient. A clinician skilled in the art can readily identify such candidate patients, by the use of, for example, clinical tests, physical examination and medical/family/travel history. Risk factors for renal cancer include aging, family history, a previous history of renal cancer, having had radiation therapy to the chest region, being Caucasian, menstruating prior to the age of 12, late menopause (after age 50), long term hormone replacement therapy, nulliparity, having children after the age of 30, and/or genetic mutations.

“After an initial period of treatment” or after an appropriate period of time after the administration of the therapeutic, e.g., 2 hours, 4 hours, 8 hours, 12 hours, or 72 hours, one or more of the cellular expression patterns may be determined again. The modulation of one or more of the cellular expression patterns may indicate efficacy of an anti-cancer treatment. One or more of the cellular expression patterns may be determined periodically throughout treatment. For example, one or more of the cellular expression patterns may be checked every few hours, days or weeks to assess the further efficacy of the treatment. The method described may be used to screen or select patients that may benefit from treatment with a therapeutic or related therapy.

The initial period of treatment may be the time required to achieve a steady-state plasma or cellular concentration of the therapeutic or related cancer treatment. The initial period may also be the time to achieve a modulation in one or more cellular expression patterns.

Treatment of a subject may entail administering more than one dose of a therapeutic in a therapeutically effective amount. Between doses, it may be desirable to determine one or more of the cellular expression patterns in the tumor after a second period of treatment with the therapeutic or related cancer treatment. This is one example how a treatment course may be monitored to determine if it continues to be efficacious for the subject when monitoring the treatment, it may be desirable to comparing one or more of the pre-treatment or post-treatment cellular expression patterns to a standard cellular expression pattern.

The present invention presents methods of treating a subject identified with renal cancer. The identification may be by diagnosis as described herein or by self-identification. The diagnosis of renal cancer may be, for example, by clinical examination, imaging procedures (e.g., ultrasound, magnetic resonance imaging (MRI)), and/or biopsy (surgical removal of tissue for microscopic examination) of a mass detected by physical examination.

A subject in need treatment for renal cancer may be treated by co-administering, radiation agent, biological agent (stem cell, antibody) or an anti-inflammatory agent to the subject. Chemotherapeutic agents may include an agent identified through the screening methods described herein, one or more of the agents linked or generated by a software program and database as PharmaProjects, or other agent determined by a health care professional.

Methods of monitoring the treatment of a subject for renal carcinoma, include, determining a pre-treatment cellular marker expression profile a cell of a subject; administering a therapeutically effective amount of a candidate compound, and determining a post-treatment cellular marker expression profile in a cell of a subject. A modulation of the a biomarker expression pattern indicates the efficacy of treatment with the a biomarker C-terminal peptide. Additional steps may also include, identifying a subject that may be retinoid unresponsive, diagnosing a subject with renal carcinoma, renal ischemia, acute renal failure, RRR, graft, and/or a subject in need of renal transplantation, and/or obtaining a cell sample from the subject.

“Cellular marker expression profile,” “pattern of expression” “expression profile” refer to determining whether or not one or more of a biomarker is expressed in a cell at a particular time, for example, pre-treatment, during treatment, or after treatment.

A method, according to the invention, to assess whether a subject who has cancer is likely to exhibit a favorable clinical response to treatment with an a biomarker therapeutic, for example, a candidate compound, comprises determining a pre-treatment expression profile of one or more biomarkers in a cell of a subject, administering a therapeutically effective amount of a candidate compound, and determining a post-treatment expression profile of the one or more biomarkers in a cell of a subject. A modulation of the a biomarker expression or the stasis of the biomarker profile following administration is an indication that the cancer is likely to have a favorable clinical response to treatment with a candidate compound.

The method of assessing whether a subject who has cancer is likely to exhibit a favorable clinical response may further comprise comparing one or more of the pre-treatment or post-treatment expression patterns of a biomarker to a standard a biomarker expression pattern. The standard a biomarker expression pattern may be the corresponding a biomarker expression pattern in a reference cell or population of cells or from normal tissue surrounding suspected cancerous tissue, or tissue from another portion of the subject, including a kidney not suspected of being cancerous.

A reference cell may be one or more of the following, cells from the subject, cultured cells, cultured cells from the subject, or cells from the subject pre-treatment. The cells may be cells from normal tissue surrounding suspected cancerous tissue, or tissue from another portion of the subject, including a kidney not suspected of being cancerous.

As used herein, “a reference cell or population of cells” refers to a cell sample that is clinically normal, clinically somewhere on the continuum between normal and neoplastic, or is neoplatic, depending on the particular methods of use. The reference cell may be one or more of the following, cells from the subject, cultured cells, cultured cells from the subject, or cells from the subject pre-treatment, for example, a sample from a different portion of the tissue being diagnosed, or it may a from another tissue of the subject. The cells may alternately be from the subject post-treatment. The reference may also be from treated tissue culture cells. The cultures may be primary or established cultures and may be from the subject being diagnosed or from another source. The cultures may be from the same tissue being diagnosed or from another tissue. The cultures may also be normal, anywhere on the continuum from normal to neoplastic, and/or neoplastic. For example, a reference cell may be a cell from the normal kidney of a subject with renal cancer.

Methods of treating renal cancer in a subject, according to the invention, include, administering a therapeutically effective amount of a candidate compound to a subject diagnosed with cancer.

The renal cancer may be at any one or more of the stages identified by a cancer staging system. A staging system is a standardized way in which the cancer care team describes the extent of the cancer. The most commonly used staging system is that of the American Joint Committee on Cancer (AJCC), sometimes also known as the TNM system (www.cancer.gov):

Screening methods, according to the invention, to identify candidate molecules to treat renal cancer, comprise contacting a cell, e.g., a cancerous cell or an ischmically injured cell, with a candidate molecule; an detecting expression pattern of a biomarker the cell, wherein expression pattern of the a biomarker in a pattern according to Table 9 indicates the molecule may be useful to treat renal cancer. Alternately, correlating the expression pattern with the patterns indicated in Table 9 indicates the renal status. The candidate molecule may be one or more of a small molecule, a peptide, or a nucleic acid. Screening methods may further comprise comparing the expression pattern to a standard expression pattern, e.g., the corresponding expression pattern in a reference cell or population of cells. A reference cell may be one or more cells from the subject, cultured cells, cultured cells from the subject, or cells from the subject pre-treatment, or a cell sample as described herein.

As used herein, “renal therapeutic,” “renal related cancer therapeutic,” “renal related cancer therapeutic,” and “Therapeutic,” are used interchangeably to indicate a compound, peptide, or other agent that is useful to treat, prevent or ameliorate renal carcinoma.

The present invention is further directed to the compounds identified by the above-described screening assays and to processes for producing such agents by use of these assays. In a preferred aspect, the renal therapeutic is substantially purified. The compounds can include, but are not limited to, nucleic acids, antisense nucleic acids, ribozyme, triple helix, antibody, and polypeptide molecules and small inorganic or organic molecules. Accordingly, in one embodiment, the present invention includes a compound obtained by a method comprising the steps of any one of the aforementioned screening assays. For example, the compound is obtained by a method comprising contacting a cell with one or more candidate molecules; and detecting expression pattern of a biomarker in the cell.

Once a test compound has been identified as having an appropriate activity according to the screening methods of the present invention, the test compound can be subject to further testing, for example, in animal models to confirm its activity as a renal related therapeutic. The test compound can also be tested against known compounds that modulate one of the parameters, in cell based or animal assays, to confirm its desired activity. The identified compound can also be tested to determine its toxicity, or side effects that could be associated with administration of such compound. Alternatively, a compound identified as described herein can be used in an animal model to determine the mechanism of action of such a compound.

The genes expressed concordantly in RRR and RCC may permit the tumor to respond to certain physiological signals that are known inhibit tissue regeneration. Therapeutic agents similar to such signaling molecules (i.e., initiation of DNA replication) could be developed and tested in the screening assays described herein.

### Cloning of Biomarkers

The term “vector” refers to a nucleotide sequence that can assimilate new nucleic acids, and propagate those new sequences in an appropriate host. Vectors include, but are not limited to recombinant plasmids and viruses. The vector (e.g., plasmid or recombinant virus) comprising the nucleic acid of the invention can be in a carrier, for example, a plasmid complexed to protein, a plasmid complexed with lipid-based nucleic acid transduction systems, or other non-viral carrier systems.

A broad variety of suitable microbial vectors are available. Generally, a microbial vector will contain an origin of replication recognized by the intended host, a promoter which will function in the host and a phenotypic selection gene such as a gene encoding proteins conferring antibiotic resistance or supplying an autotrophic requirement. Similar constructs will be manufactured for other hosts. E. coli is typically transformed using pBR322. See Bolivar et al., Gene 2, 95 (1977). The vector pBR322 contains genes for ampicillin and tetracycline resistance and thus provides easy means for identifying transformed cells. Expression vectors should contain a promoter which is recognized by the host organism. This generally means a promoter obtained from the intended host. Promoters most commonly used in recombinant microbial expression vectors include the beta-lactamase (penicillinase) and lactose promoter systems (Chang et al., Nature 275, 615 (1978); and Goeddel et al., Nucleic Acids Res. 8, 4057 (1980) and EPO Application Publication Number 36,776) and the tac promoter (H. De Boer et al., Proc. Natl. Acad. Sci. USA 80, 21 (1983)).

The isolated nucleotide sequences of the invention may be cloned or subcloned using any method known in the art (See, for example, Sambrook, J. et al., Molecular Cloning, Cold Spring Harbor Press, New York, 1989), the entire contents of which are incorporated herein by reference. In particular, nucleotide sequences of the invention may be cloned into any of a large variety of vectors. Possible vectors include, but are not limited to, cosmids, plasmids or modified viruses, although the vector system must be compatible with the host cell used. Viral vectors include, but are not limited to, lambda, simian virus, bovine papillomavirus, Epstein-Barr virus, and vaccinia virus. Viral vectors also include retroviral vectors, such as Amphatrophic Murine Retrovirus (see Miller et al., Biotechniques, 7:980-990 (1984)), incorporated herein by reference). Plasmids include, but are not limited to, pBR, PUC, pGEM (Promega), and Bluescript® (Stratagene) plasmid derivatives. Introduction into and expression in host cells is done for example by, transformation, transfection, infection, electroporation, etc.

Conventional procedures were also used to make vector DNA, cleave DNA with restriction enzymes, ligate and purify DNA, transform and/or transfect host cells, culture the host cells, and isolate and purify proteins and polypeptides. See generally Sambrook et al., Molecular Cloning (2d ed. 1989), and Ausubel et al. supra. Examples of cells which can express isolated DNAs encoding the antibodies disclosed herein include bacterial cells (e.g., E. coli and B. subtilis) such as, e.g., M94, DM52, XL1-blue (Stratagene), animal cells (e.g., NSO, CV-1, CHO cells), yeast cells (e.g., S. cerevisiae), amphibian cells (e.g., Xenopus oocyte), and insect cells (e.g., Spodoptera fugiperda or Trichoplusia ni). Methods of expressing recombinant DNA in these cells are known, e.g., see Sambrook et al., Molecular Cloning (2d ed. 1989), Ausubel et al. supra, and Summer and Smith, A Manual of Methods for Baculovirus Vectors and Insect Cell Culture Procedures: Texas Agricultural Experimental Station Bulletin No. 1555, College Station Texas (1988).

A vector, according to the invention, may contain a polynucleotide capable of encoding a polypeptide having at least about 80% sequence identity to the sequences, and characterized by the ability to alter the expression pattern of a biomarker. The encoded polypeptide may also be at least 85%, 90%, 95%, or 99.9% identical to at least one of the sequences identified herein. A vector according to the invention may encode more than one polynucleotide capable of encoding a peptide characterized by he ability to alter the expression pattern of a biomarker, for example, the vector may encode two, three or four polynucleotides capable of encoding a peptide characterized by he ability to alter the expression pattern of a biomarker.

Preferably the a biomarker polynucleotide of the invention is derived from a mammalian organism, and most preferably from human. Screening procedures which rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code., however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the biomarker DNA to that single probe in the mixture which is its complete complement (Wallace, et al., Nucl. Acid Res., 9:879, 1981; Maniatis, et al., Molecular Cloning: A Laboratory Manual, Cold Spring Harbor, N.Y. 1989).

The development of specific DNA sequences encoding a biomarker can also be obtained by: 1) isolation of double-stranded DNA sequences from the genomic DNA; 2) chemical manufacture of a DNA sequence to provide the necessary codons for the polypeptide of interest; and 3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA.

DNA sequences encoding a biomarker can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

Polynucleotide sequences encoding a biomarker can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention. Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired. Isolation and purification of microbial expressed polypeptide, or fragments thereof, provided by the invention, may be carried out by conventional means including preparative chromatography and immunological separations involving monoclonal or polyclonal antibodies. The a biomarker polypeptides of the invention can also be used to produce antibodies which are immunoreactive or bind to epitopes of the a biomarker polypeptides. Antibody which consists essentially of pooled monoclonal antibodies with different epitopic specificities, as well as distinct monoclonal antibody preparations are provided. Monoclonal antibodies are made from antigen containing fragments of the protein by methods well known in the art (Kohler, et al., Nature, 256:495, 1975; Current Protocols in Molecular Biology, Ausubel, et al., ed., 1989).

The identification of a novel member of the a biomarker family may provide useful tools for diagnosis, prognosis and therapeutic strategies associated with a biomarker mediated disorders. Methods of identifying a biomarker family members are well known to one of skill in the art.

### Pharmaceutical Compositions

The present invention also provides pharmaceutical compositions. Such compositions comprise a therapeutically effective amount of at least one therapeutic, (e.g., a renal related therapeutic), and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the renal related therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water can be a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated, in accordance with routine procedures, as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The therapeutics of the invention can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

Preferred pharmaceutical compositions and dosage forms comprise a therapeutic of the invention, or a pharmaceutically acceptable prodrug, salt, solvate, or clathrate thereof, optionally in combination with one or more additional active agents.

The amount of the therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 1-50 milligrams of active compound per kilogram body weight. Suitable dosage ranges for intranasal administration are generally about 0.1 mg/kg body weight to 50 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

The therapeutics of the present invention may also be administered by controlled release means or delivery devices that are well known to those of ordinary skill in the art, such as those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566. These controlled release compositions can be used to provide slow or controlled-release of one or more of the active ingredients therein using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable controlled-release formulations known to those of ordinary skill in the art may be readily selected for use with the pharmaceutical compositions of the invention.

Controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations may include extended activity of the drug, reduced dosage frequency, and/or increased patient compliance.

Most controlled-release formulations are designed to initially release an amount of the therapeutic that promptly produces the desired therapeutic effect, and gradually and continually releases other amounts of the therapeutic to maintain the appropriate level of therapeutic effect over an extended period of time. In order to maintain this constant level of therapeutic in the body, the therapeutic must be released from the composition at a rate that will replace the amount of therapeutic being metabolized and excreted from the body. The controlled-release of the therapeutic may be stimulated by various inducers, for example, pH, temperature, enzymes, water, or other physiological conditions or compounds. Such controlled-release components in the context of the present invention include, but are not limited to, polymers, polymer matrices, gels, permeable membranes, liposomes, microspheres, or the like, or a combination thereof, that facilitates the controlled-release of the active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

A therapeutic agent can be co-administering with one or more of a chemotherapeutic agent, a biomarker ligand, RAR selective ligand, radiation agent, hormonal agent (e.g., megestrol acetate), biological agent (e.g., stem cell, antibody) or an anti-inflammatory agent to the subject. Chemotherapeutic agents may be one or more of tamoxifen, trastuzamab (herceptin), raloxifene, doxorubicin, fluorouracil/5-fu, pamidronate disodium, anastrozole, exemestane, cyclophos-phamide, epirubicin, letrozole, toremifene, fulvestrant, fluoxymester-one, trastuzumab, methotrexate, megastrol acetate, docetaxel, paclitaxel, testolactone, aziridine, vinblastine, capecitabine, goselerin acetate, zoledronic acid, and/or taxol.

Compounds that may be co-administered with therapeutic agents include steroid or a non-steroidal anti-inflammatory agent. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof. For a more detailed description of the NSAIDs, see Paul A. Insel, Analgesic-Antipyretic and Antiinflammatory Agents and Drugs Employed in the Treatment of Gout in Goodman & Gilman's The Pharmacological Basis of therapeutics 617-57 (Perry B. Molinhoff and Raymond W. Ruddon eds., 9^{th} ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

Other examples of agents that may be co-administered include, but are not limited to, immunomodulatory agents, anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, non-steriodal anti-inflammatory drugs (e.g., aspirin, ibuprofen, diclofenac, and COX-2 inhibitors), and leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

Other compounds that may be co-adminstered with an a biomarker directed therapy include, anti-bacterial, anti-fungal, anti-viral, anti-hypertension, anti-depression, anti-anxiety, and anti-arthritis substances, as well as substances for the treatment of allergies, diabetes, hypercholesteremia, osteoporosis, Alzheimer's disease, Parkinson's disease, and/or other neurodegenerative diseases, and obesity. Specific categories of test substances can include, but are not limited to, PPAR agonists, HIV protease inhibitors, anti-inflammatory drugs, estrogenic drugs, anti-estrogenic drugs, antihistamines, muscle relaxants, anti-anxiety drugs, anti-psychotic drugs, and anti-angina drugs. Other drugs may be co-administered with a biomarker related therapies according to the needs of a particular subject.

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

The foregoing and other useful combination therapies will be understood and appreciated by those of skill in the art. Potential advantages of such combination therapies include the ability to use less of each of the individual active ingredients to minimize toxic side effects, synergistic improvements in efficacy, improved ease of administration or use and/or reduced overall expense of compound preparation or formulation. The biological activities of a compound of this invention can be evaluated by a number of cell-based assays.

In combination therapy treatment, both the compounds of this invention and the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the other therapeutic agent's optimal effective-amount range. In one embodiment of the invention where another therapeutic agent is administered to an animal, the effective amount of the compound of this invention is less than its effective amount would be where the other therapeutic agent is not administered. In another embodiment, the effective amount of the conventional agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In various embodiments, the therapies (e.g., prophylactic and/or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patent visit.

In certain embodiments, one or more compounds of the invention and one or more other therapies (e.g., prophylactic or therapeutic agents,) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same compounds of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than a compound of the invention may be repeated and the administration may be separated by at least at least 1 day, 2 days, 3 days, 5 days, 10 days, 15 days, 30 days, 45 days, 2 months, 75 days, 3 months, or at least 6 months.

Formulations and methods of administration that can be employed when the Therapeutic comprises a modulating compound identified by the assays described, supra; additional appropriate formulations and routes of administration can be selected from among those described herein below. Moreover, a Therapeutic of the invention can be also be administered in conjunction with any known drug to treat the disease or disorder of the invention.

The gene product and/or the nucleic acid of discordantly expressed genes are potential drug candidates. For example, a gene product that is expressed in normal tissue, but not in injured tissue is a particularly attractive drug candidate that may be screened with the methods described herein.

### Kits

In yet another aspect, the present invention provides kits for qualifying renal status, wherein the kits can be used to measure the markers of the present invention. For example, the kits can be used to measure any one or more of the markers described herein, which markers are differentially present in samples of renal cancer patient, ischemically injured subjects, and normal subjects. The kits of the invention have many applications. For example, the kits can be used to differentiate if a subject has renal cancer or has a negative diagnosis, thus enabling the physician or clinician to diagnose the presence or absence of the cancer. The kits can also be used to monitor the patient's response to a course of treatment, enabling the physician to modify the treatment based upon the results of the test. In another example, the kits can be used to identify compounds that modulate expression of one or more of the markers in in vitro or in vivo animal models for renal cancer.

The present invention therefore provides kits comprising (a) a capture reagent that binds a biomarker selected from Table 9; and (b) a container comprising at least one of the biomarkers. In preferred kit, the capture reagent binds a plurality of the biomarkers. In certain preferred embodiments, the kit of further comprises a second capture reagent that binds one of the biomarkers that the first capture reagent does not bind.

Further kits provided by the invention comprise (a) a first capture reagent that binds at least one biomarker selected from those listed in Table 9, and (b) a second capture reagent that binds at least one of the biomarkers that is not bound by the first capture reagent. Preferably, at least one of the capture reagents is a nucleic acid.

While the capture reagent can be any type of reagent, preferably the reagent is a complementary nucleic acid probe.

The invention also provides kits comprising (a) a first capture reagent that binds at least one biomarker selected from Table 9, and (b) instructions for using the capture reagent to measure the biomarker. In certain of these kits, the capture reagent comprises a complementary nucleic acid probe. One embodiment of the present invention includes a high-throughput test for early detection of renal cancer, which analyzes a patient's sample on the nucleic acid chip array.

In other embodiments, the kits as described herein comprise at least one capture reagent that binds at least one biomarker selected from the markers listed in Table 9 an/or the markers of clusters 1-27.

Certain kits of the present invention further comprise a wash solution, or eluant, that selectively allows retention of the bound biomarker to the capture reagent as compared with other biomarkers after washing. Alternatively, the kit may contain instructions for making a wash solution, wherein the combination of the adsorbent and the wash solution allows detection of the markers using gas phase ion spectrometry.

Preferably, the kit comprises written instructions for use of the kit for detection of cancer and the instructions provide for contacting a test sample with the capture reagent and detecting one or more biomarkers retained by the capture reagent. For example, the kit may have standard instructions informing a consumer how to wash the capture reagent (e.g., probe) after a sample of blood serum contacts the capture reagent. In another example, the kit may have instructions for pre-fractionating a sample to reduce complexity of proteins in the sample. In another example, the kit may have instructions for automating the fractionation or other processes.

Such kits can be prepared from the materials described above, and the previous discussion of these materials (e.g., probe substrates, capture reagents, adsorbents, washing solutions, etc.) is fully applicable to this section and will not be repeated.

In another embodiment, a kit comprises (a) an antibody that specifically binds to a marker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with a diagnosis of renal cancer.

The present invention also provides a screening assay comprising (a) contacting a cancer cell with a test agent and (b) determining whether the test agent modulates the activity of any one or more of the biomarkers listed in Table 9. The biomarkers of Table 9 include any of the discordantly or concordantly expressed genes between the RRR and RCC models and normal cells. The examples below and Tables show numerous examples of biomarkers that are useful for screening assays.

Kits, according to the invention, may include reagents, including primers, polymerases, antibodies, buffers, nucleic acid chips, protein chips, antibody chips and/or labels. The kit may also include, microscope slides, reaction vessels, instruction for use of the reagents and material and how to interpret the data generated from the assays. For example, PCR primers for the amplification of the a biomarker transcript may also be included. Antibodies to detect the a biomarker proteins may also be included in the kit.

### EXAMPLES

It should be appreciated that the invention should not be construed to be limited to the examples which are now described; rather, the invention should be construed to include any and all applications provided herein and all equivalent variations within the skill of the ordinary artisan.

### Example 1

Using gene expression profiling, we investigated in a rodent model the gene expression changes relative to normal kidney, occurring after ischemia/reperfusion injury and during the first two weeks of RRR. Consequently, a detailed analysis revealed distinct regenerative gene expression patterns, pathways, transcriptional control and gene functions. The RRR differential gene expression was then qualitatively compared with the global gene expression of RCC as opposed to human normal kidney. Two distinct signatures were revealed: (1) a substantial concordant overlap reflecting the normal regenerative phenotype, and (2) a divergent discordant (inverted) pattern of expression where gene expression changes are in opposite direction in RRR and RCC.

Animals

The mice were 5-week-old C57BL/6 female mice (60 to 100 g) and obtained from the National Institute of Health (NIH). The animals had free access to water and food. Animal care and experiments were performed with the approval of the Animal Care and Use Committee of the National Cancer Institute, Maryland.

Ischemia-Reperfusion Model

Regeneration was induced by the renal warm ischemia method (Chiao H 1997, Chiao H 1998). Mice were anesthetized with ketamine, xylazine, and acepromazine and placed on a heating table kept at 37° C. to maintain constant body temperature. A left unilateral flank incision was made, the left kidney perirenal fat removed, and the left renal artery exposed. A non-traumatic vascular clamp was placed across the renal artery for 50 minutes. After removal of the clamp, the kidney was inspected for restoration of blood flow, and 1 ml of pre-warmed (37° C.) normal saline was instilled into the abdominal cavity. The abdomen was closed with wound clips (Roboz Surgical Instrument Co., Inc, RS-9262), and the animals were allowed to recover in a 37° C. incubator. After the desired period of reperfusion (0, 6, and 12 hours and on days 1, 2, 5, 7 and 14), the animals were anesthetized and both kidneys were rapidly excised by midline abdominal incision. For microarray studies, the kidneys were flash frozen in liquid nitrogen and stored at −70° C. For histological studies, the kidneys were bivalved with a coronal cut and fixed in formalin (10%). Normal and ischemic kidneys were removed, processed, and frozen in an identical manner.

Immunohistochemistry

Fixed and paraffin-embedded tissue specimens were deparaffinized, rehydrated, subjected antigen unmasking (Morgan J M et al 1994), and treated to nonspecific block staining. For this latter procedure, sections were incubated for 20 min at 24° C. with 1% H₂O₂ in methanol, followed by blocking for 30 min with 5% normal horse serum in PBS. Polyclonal antibodies against Ki67 (NOVO, NCL-Ki67p) or mouse glucose transporter (Glut-1) (Alpha Diagnostic Intl; GT11-A) were added (1:1000 dilution) for 16 h at 4° C., followed by incubation for 30 min at room temperature with biotinilated secondary goat anti-rabbit IgG antibodies and 30 min with avidin-biotin peroxidase conjugate (1:50 dilution) (Vectastain Elite Universal kit: Vector Laboratories, Burlingame, Calif.). Color was developed using Vector Labs 3,3-Diaminobenzidine kit for 10 min followed by counterstaining with Mayer's hematoxylin. Negative controls were performed using nonimmnune serum or PBS. Three investigators independently evaluated the immunohistochemistry.

### Microarray Procedures

Mouse cDNA microarrays (NIH/NCI GEM2) containing 9646 cDNA spots were used to quantitate mRNA expression in the kidney samples. A reference probe consisting of an equal mixture of 6 normal mouse tissues (brain, heart, kidney, liver, lung and spleen) was used in the competitive hybridization experiments. For the reference probe 50 ug of total RNA were reverse transcribed, and to avoid an amplification step for the experimental sample, 3.0 ug of poly(A)+ RNA were subjected to oligo(dT)-primed reverse transcription. The remaining procedures were performed as described previously (Rosenwald et al., 2002). See Table 9.

### Quantitative Real-Time RT-PCR

RNA was isolated using Trizol Reagent (Invitrogen, California). Total RNA (1 g) was reverse transcribed in a volume of 50 μl. 5 μl of the resulting solution was then used for PCR according to the manufacturer's instructions (Applied Biosystems, Foster City, Calif.). Gene expression for IGFBP1, IGFBP3, CTGF, AKT, FRAP, MYC, NF-κB, HK1 and SIRT7 were quantified relative to the expression level of ribosomal 18s. PHD1, PHD2 and PHD3 were quantified relative to the expression level of filamin B, (actin binding protein 278; FLNB{tilde over ())} All probes were purchased from Applied Biosystems, Inc. (Foster City, Calif.). Normalized data are presented as-fold difference in log₂ gene expression.

### Motif Selection

Statistical analysis of transcription factor binding sites in the current set of up- and down-regulated genes. We retrieved 1-kb sequences in the upstream region of the genes for 523 up- and 318 down-regulated genes (a subset of 1325 up/down genes). The 1-kb sequences in the promoter regions were used to search for transcription factor (TF) binding sites using a TransFac web server. To identify TF binding sites enriched in the set of up- or down-regulated genes, we used Fisher's exact test to search TF sites that differed significantly between the up- and down-regulated genes. We constructed a 2×2 table with up/down genes and presence/absence of TF sites for each of the 177 TF sites (see Method). Four p-value cutoffs were used to select up/down genes and fisher's test was used to test each table.

### Analysis Of Currated Pathway Genes

Using PubMed, a survey of the literature published from 1966 through mid 2003 was performed, and differentially expressed genes in the following categories were extensively catalogued: RCC vs. normal kidney; renal cell culture hypoxia responsive genes vs. normoxia-responsive genes; HIF-regulated genes; VHL, IGF, MYC, NF-kB pathway genes; purine pathway genes; genes expressed following renal ischemia reperfusion and/or ARF vs. genes expressed in normal kidney; and the tissue expression pattern of renal genes (e-renal histology). The gene datasets were translated into a distinct set of gene identifiers (i.e., the HUGO gene symbol) that were used to facilitate cross comparisons among datasets. Only genes that were printed on the GEM2 microarray were considered for further analysis (differentially expressed and unchanged expression).

To navigate among gene identifiers, the programs MatchMiner (http://discover.nci. nih.gov/matchminer/html/index.jsp) and SOURCE http://source.stanford.edu) were used.

The enrichment of genes in various pathways in concordant or discordant groups was analyzed by using the chi square test (tables 3, 4 and 12). An example of 2×2 contingency table is shown immediately below:

| | Concord | Remainder |
|---|---|---|
| Hypoxia pathway | 35 | 216 |
| Remainder | 243 | 5302 |

251 genes were mapped to the hypoxia pathway and printed on the GEM2 array, 35 of which showed concordant expression with a remainder of 216 in the first row. A total of 278 genes are located in the first column, 35 of which showed concordant expression with a remainder of 243. 5,796 genes were on the microarray, producing a remainder of 5302 genes in column 2 (5796-35-216-243). The p-value for the 2×2 table was calculated using Statistic Package R.

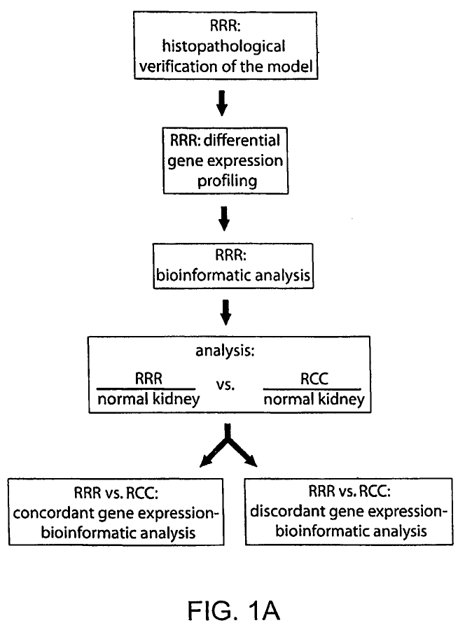

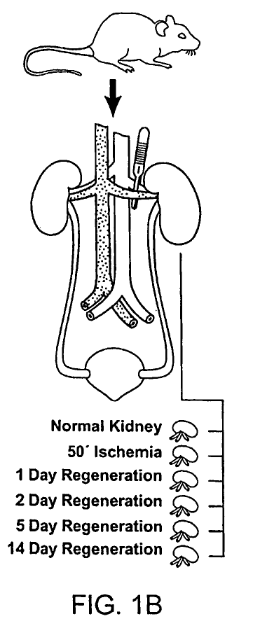

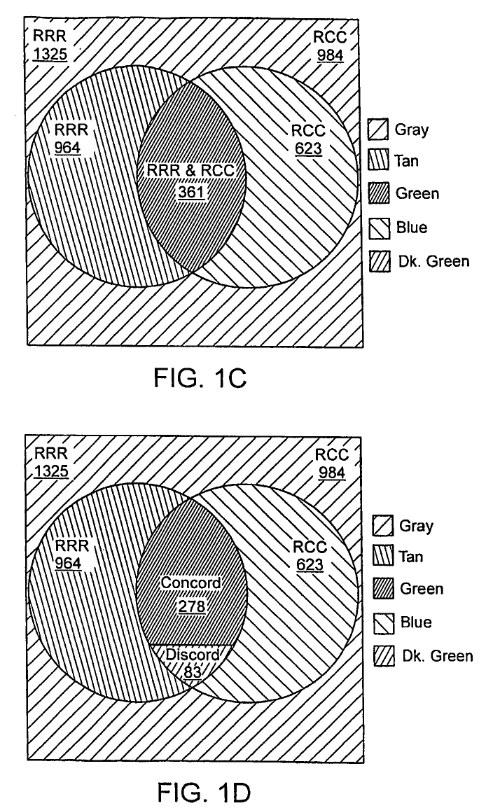

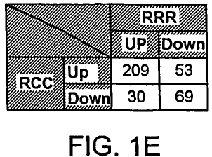

In order to establish an understanding of the process of renal regeneration repair (RRR) and its relationship to the gene expression changes in renal cell carcinoma (RCC), we first characterized histopathological changes and differential gene expression as a consequence of 50 minutes warm ischemia in a murine model of renal RRR (FIG. 1), (Suparvekin S. et al 2003). We then compared the gene expression patterns, pathways, transcriptional control and gene functions of RRR to RCC. To accomplish this study, the following five steps were performed and are described bellow: (1) characterization of the process of RRR by temporal histopathology changes; (2) characterization of the differential gene expression as a consequence of RRR; (3) Identification of specific functional gene-clusters by ontology analysis, probabilistic functional genomics and cross-comparison with the pathway literature; (4) identification of similarities and differences in gene expression between RRR and RCC; (5) analysis of biological meaning of concordant and discordant genes associated with RRR and RCC.

### Characterization of the Histopathology of RRR

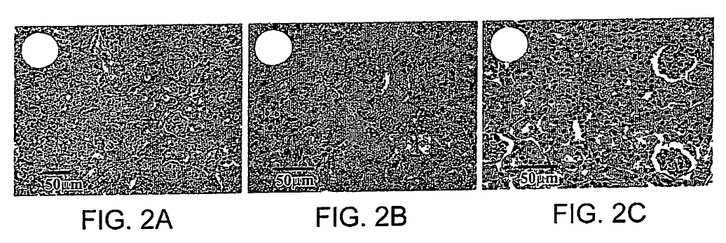

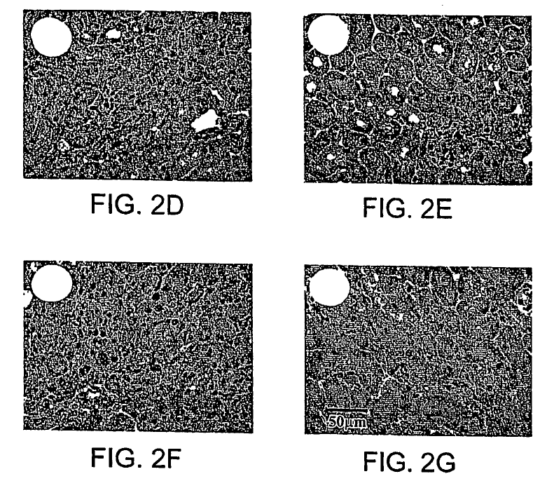

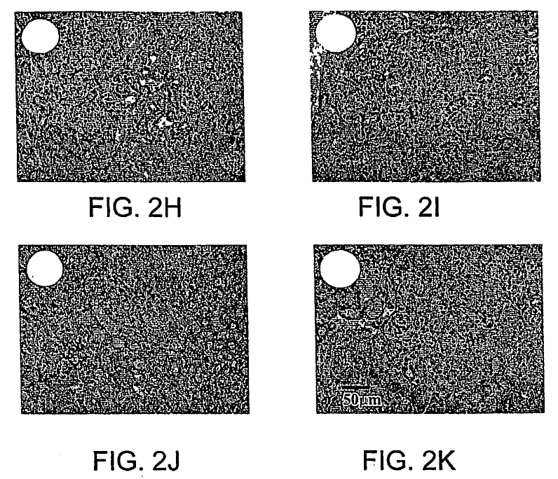

Early histopathologic features of ischemic injury induced by 50 minutes of vascular clump were readily evident in the kidney within the first 12 hours of reperfusion and were monitored at 1, 2, 5, 7 and 14 days. As expected, we observed apoptotic cells in the outer medulla within 12 hours of reperfusion, which became more abundant over the first 24 hours following initial injury (Suparvekin S. et al 2003) (data not shown). At one day after the ischemic event, more than half of cortical tubules (FIG. 2C) showed some degree of staining for glucose transporter-1 (Glut-1/SLC2A1), which is regulated by the transcription factor hypoxia-inducible factor 1 (HIF1). Up-regulation of HIF1 provides tissue protection from ischemic damage during the early regeneration phase (Matsumoto M. et al 2003). At 2 days, we observed by hematoxylin and eosin (H&E) staining an acute tubular necrosis in which about half of the tubules showed necrosis with loss of epithelium; the remaining tubules showed cells with reactive nuclear changes (hyperchromasia, prominent nucleoli) (FIGS. 2A, 2B). At 2 days, the necrotic-apoptotic events were accompanied by positive tubules staining with the proliferation marker MiB-1 (FIG. 2B). At two weeks, most tubules showed a normal appearance with only rare examples showing degenerative or regenerative changes (FIG. 2B). Thus, the histological evidence reported here supports the accepted process of renal injury, regeneration, and recovery (Sutton T A et al 2002). Damaged renal tissue is first characterized by regenerating tubules in which necrotic cells are accompanied by replicating cells; at two weeks, most tubules have recovered and regained their normal appearance.

### Characterization of Differential Gene Expression as a Consequence of Renal IRI: Defined Phases of Early, Late and Continuous Tissue Regeneration

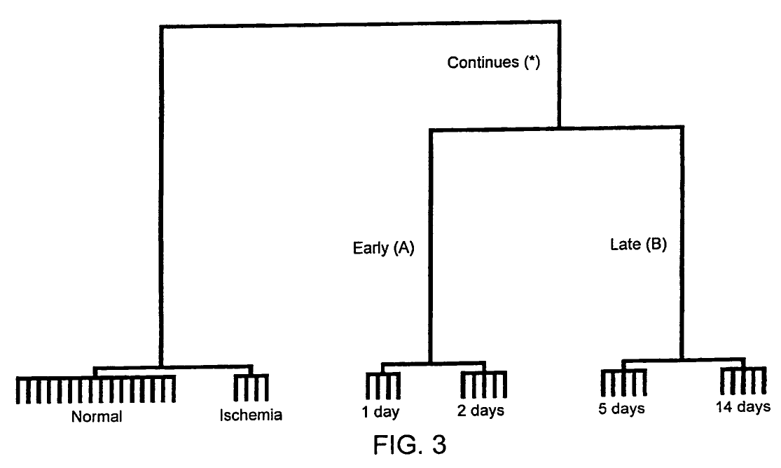

Employing cDNA microarray analysis of 9,646 genes, we were able to compare the changes in the global pattern of gene expression of normal (day 0), ischemic (50 minutes) and reperfused (at 1, 2, 5 and 14 days) kidney issue. A differential expression pattern was observed for a group of 1,350 gene spots, corresponding to 1,325 genes (P-value ≦0.05). This differential pattern clustered into a dendrogram consisting of four main branches (FIG. 3, 1s). The first branch included the normal and ischemic kidney tissue; the second branch included genes accompanying regenerative processes taking place continuously throughout the two-week period (FIG. 3 marked as asterisk); the third branch was of genes expressed during early regenerative processes taking place during the first two days following reperfusion (FIG. 3 marked as A); and finally, the fourth branch included genes expressed late, at 5 and 14 days after reperfusion (FIG. 3 marked as B).

The differential expression of each gene was averaged and calculated as relative to the same gene expressed in normal and ischemic kidney tissues. All the repetitive samples clustered together, illustrating the reproducibility of the animal model and supporting the reliability of the array methodologies employed. Therefore, relative to the normal kidney, we identified three phases of RRR: continuous, early and late.

Of the 1,325 RRR genes that were differentially expressed from normal kidney during the first two weeks, 323 genes were continuously differentially expressed throughout the period (189 up-regulated and 134 genes down-regulated); in the early phase of RRR, 629 were differentially expressed (336 up-regulated and 293 down-regulated) and in the late phase of RRR, 373 genes were differentially expressed (227 were up-regulated and 96 down-regulated), (Table 1). Table 1 summarizes the data related to the amount of genes that were differentially expressed and are therefore of potential functional importance in general biological processes involved in RRR. A complete listing of all genes is given in Table 9.

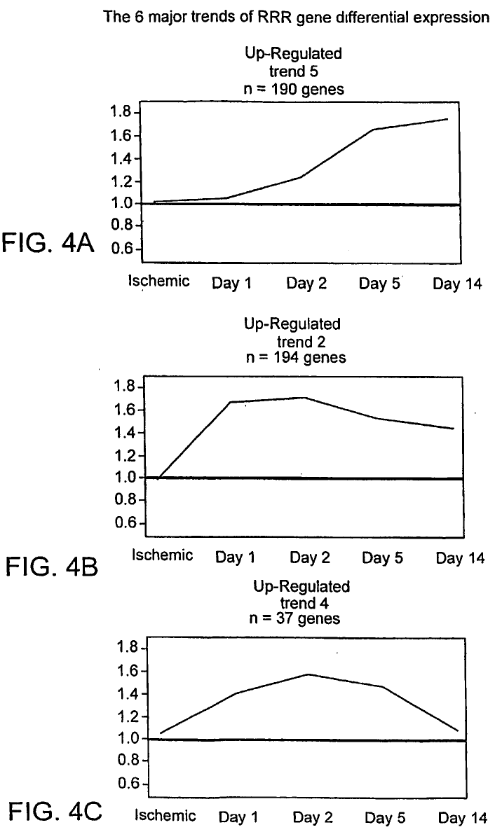

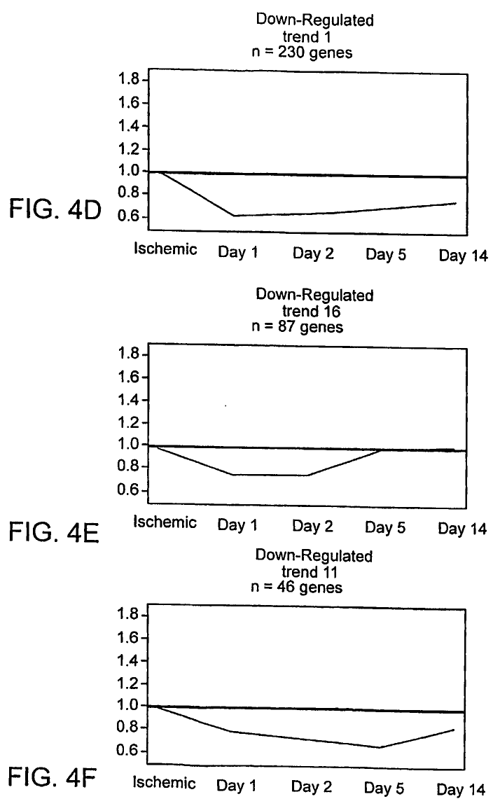

The RRR differential gene expression as opposed to normal kidney was further clustered to identify different temporal patterns/trends. We statistically identified 27 trends. Trend 1 (FIG. 4A) represents the major patterns of genes that were down-regulated during RRR and partially returned towards normal levels, by day 14, (n=270). Trend 2 or 4 (FIG. 4B) is the pattern seen for 199 genes that were up-regulated at the early phase (days 1 and 2) and reduced towards normal levels at the late phase (days 5 and 14). Trend 5 (FIG. 4C) represents 190 genes that were early up-regulated and remained up-regulated on the 14th day of RRR. Trend 16 (FIG. 4D) contains 87 genes that were down-regulated at days 1 and 2, but were back to normal levels on day 5. Other patterns are discerned statistically, but follow similar tendency as the representative trends shown, which contain the majority of the differentially expressed genes.

Identification of Specific Functional Gene-Clusters by Ontology Analysis, Probabilistic Functional Genomics, and Cross-Comparison with the Pathway Literature

The gene expression of RRR phases according to biological processes, molecular functions, and cellular expression patterns by gene ontology (http://www.geneontology.org) was analyzed. The analysis is summarized in Table 10.

During the early phase, the unique ontologies with a majority of up-regulated genes were either DNA replication or entrance into the S-phase of the mitotic cell cycle. Ontologies of a majority of early phase, down-regulated genes were oxidative phosphorylation, metabolism, growth factor binding and. Both up- and down-regulated early phase genes were regulators of translation, cell growth, and/or cell maintenance-all processes that are required for cell survival and growth (Table 10).

During the late phase, after tissue regeneration began, the biological processes associated with a majority of up-regulated genes were related to inflammation and catabolism at the proteasome core complex, microfibril and the ECM. These late, up-regulated genes modulated several distinct molecular functions—MHC class I receptor activity, collagenase activity, phospholipase inhibitor activity, hydrolase activity-actions on carbon-nitrogen (but not peptide) bonds, apoptosis inhibitor activity, peptidase activity, and receptor activity. Biological processes associated with both late up- and down-regulated genes were mainly urea cycle intermediate metabolism and the response to wounding (Table 10).

Throughout the entire RRR process, ontologies with a majority of continuously up-regulated genes were of ribosome biogenesis and assembly; protein biosynthesis; cytoplasm organization; biogenesis; and biological responses to abiotic (non-living) stimulus. Continuously up-regulated genes were associated with molecular functions that included immunoglobulin binding, chemokine activity, G-protein-coupled receptor binding actin binding, RNA binding, and finally, processes accompanying the defense response following injury, which are also significant during the late phase of RRR. The ontologies associated with a majority of continuously down-regulated genes were related to the processes of phenylalanine metabolism and catabolism as well as fatty acid metabolism, which was also significant during the early phase of RRR. The continuously down-regulated genes were associated with the function of anion transporter activity; and oxidoreductase activity, the latter of which is also significant during the early phase. The continuously phase ontologies with both up- and down-regulated genes were of inorganic anion transport; posttranslational membrane biomarkering, blood coagulation, endoplasmic reticulum (ER) organization, and biogenesis. The cellular components that were affected during the continuous phase included the cytosolic ribosome, the actin filament, the ECM and the mitochondrion (Table 2, 3-supplement).

To further understand the relationships from the current 1325 RRR differentially expressed genes with the literature databases and genome-wide promoter analysis, we reviewed the evidence reported in the literature on the pathways and regulators previously described in both RRR and RCC: The pathways of focus for detailed analysis were in respect to the VHL tumor suppressor, and included hypoxia, interacting proteins and biomarker genes of VHL, HIFs (HRE), Myc, p53, NF-kB and IGF (Elson D. A. et al., 2000, Maxwell P H. 2004, Schips L et al 2004, Hammerman M R 1999, Yamaguchi S et al 2003, Koshiji M et al 2004, Schmid T et al 2004, Qi H and Ohh M2004, Cao C C et al 2004). The VHL pathway database included 865 genes of which 341 genes were printed on the GEM2 array and 104 genes were differentially expressed. The VHL database included interacting proteins and genes that differentially expressed dependently of the VHL in renal cells and dependent or not on oxygen (Table 9). The database of the hypoxia regulated genes included 551 genes regulated by hypoxia of which 251 genes were printed on the GEM2 array and 95 genes were differentially expressed. Of the hypoxia regulated genes in our database, the promoter of 45 genes included an HRE, 39 were printed on the array and of which 17 were differentially regulated (Table 9). The Myc pathway included 728 genes including biomarker gene and interacting proteins. 368 genes of the Myc pathway database were printed on the GEM2 array of which 136 were differentially expressed (Table 9). The p53 pathway dataset included 2,808 genes including p53 biomarker genes of cell adhesion, cell cycle, miscellaneous, structural, tumor suppressor/apoptosis, GDT/GTP binding, growth factors and hormone, lymphocyte signaling, Membrane receptor, neurobiology, protein kinase, protein phosphatase, steroid receptor and transcription regulation (Hoh J et al (2002)), (Table 9). 1259 genes of the p53 pathway database were printed on the GEM2 array and of which 262 were differentially expressed. The NF-κB pathway database included 446 genes that included biomarker genes, inducers, interacting proteins and inhibitors. 200 of these genes were printed on the GEM2 array and of which 52 genes were differentially expressed (Table 9). The IGF pathway database included 306 genes as biomarker genes, inducers, interacting proteins and inhibitors of which 139 genes were printed on the GEM2 array and 52 were differentially expressed (Table 9).

The comparison of the 1325 RRR differentially expressed genes with genes in these pathways was significantly (p<0.05) associated with the pathways of VHL, hypoxia, HIF1a (HRE) and Myc. Biomarker genes and regulators in the pathways of IGF, p53 and NF-kB were also evident, but with association significance of p>0.05 for the whole 1325 RRR differentially expressed genes (Table 4).

We next compared the up-regulated (189 genes) and down-regulated (134 genes) genes of the current RRR dataset with the genes in the pathways associated with VHL gene. Genes in both sub-sets played significant roles (p<0.05) as components of pathways associated with VHL, Myc, p53 and NF-kB. As subsets of the 1,325 genes, the up- or down-regulated genes were evident, but with association significance of p>0.05, for pathways associated with Hypoxia, or HIF (HRE) (Table 4, 1-supplement).

### Similarities and Differences Between RRR and RCC

We next investigated similarities and differences between gene expression associated with RRR and those reported to be associated with RCC. We extensively surveyed the literature and cataloged 984 genes expressed differentially in RCC as relative to normal kidney (Table 1-supplement) (Riss et al., 2004 review in preparation). Then RCC dataset was qualitatively cross-compared with the differential expression of the current set of 1,325 RRR genes as relative to normal kidney.

The analysis revealed a group of 361 genes that matched both the experimental RRR dataset and the RCC literature (FIG. 4A, Table 9). Of these 361 genes, 285 genes (77%) were concordantly expressed in both RRR and in RCC; 209 genes were up-regulated (i.e. VCAM1, ICAM1, MYC, MP14, MDM2, STAT3, ID2, TIMP1, CD44, ITGB1 and AKT1), (P<0.001), while 69 genes were down-regulated (P<0.001) both in RRR and in RCC (i.e. EGF, JUP, SDHB, SLC12A1, and CALB1), (FIG. 4B, Table 9).

Previous reports suggested that RRR and or RCC subject to regulation by hypoxia and a number of pathways as VHL, HIF, IGF, Myc, p53 and NF-kB (Elson D. A. et al., 2000, Maxwell P H. 2004, Schips L et al 2004, Hammerman M R 1999, Yamaguchi S et al 2003, Koshiji M et al 2004, Schmid T et al 2004, Qi H and Ohh M2004, Cao C C et al 2004). We therefore tested if biomarker genes of these pathways or their regulators were significantly found in the 285 concordantly expressed genes. In both RRR and RCC the concordant genes significantly (p<0.05) included genes regulated by hypoxia and pathways as VHL, Myc, p53 and NF-kB. HIF and IGF pathway genes were also evident among the concordant genes but with association significance of p>0.05.

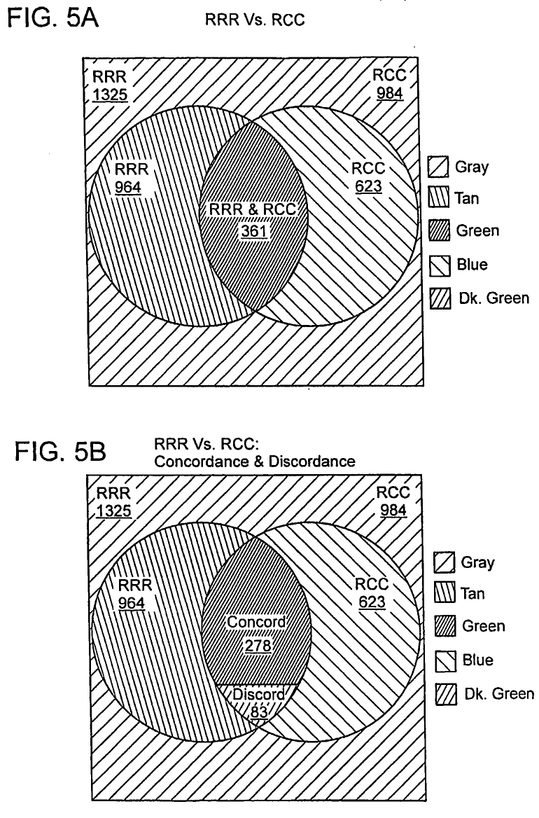

The concordant genes were significantly (p<0.05) expressed in six of the temporal patterns/trends of gene expression and included the up-regulated trends: 2, 4, 6, 14 and the down-regulated trends 1 and 16 (Table 6—supplement; FIG. 5). Further, trends 1, 4, 6 and 14 were significant to the concordant genes and not to the discordant one (the temporal patterns/trends of gene expression are described in the Characterization of differential gene expression as a consequence of renal Ischemia) (Table 6-supplement).

The remainder of the 361 genes, 83 genes (23%), were discordantly expressed during RRR as compared to RCC. Of these 83 discordant genes, 30 genes were in RRR up-regulated and in RCC down-regulated (P<0.001). The remaining 53 genes were down-regulated in RRR and up-regulated in RCC(P<0.001). Of significance (p<0.05) were genes in the pathways of VHL, hypoxia, HIF1a (HRE), IGF, and p53. HIF and IGF pathways are significantly unique to the discordant genes and not for the concordant genes. On the other hand, genes in the NF-κB pathway were significant for the concordant genes, but only evident among the discordant genes, with association significance of p>0.05.

Three temporal patterns/trends of gene expression, down-regulated trends 2, 11, and the up-regulated trend 16, significantly included discordant genes (p<0.05). Trend 11 was significantly unique to the discordant genes and not the concordant genes. Trend 11 trend encompassed 46 down-regulated genes (9 of which were discordantly expressed) active from the first day until the fifth day of RRR, when they began to return to normal levels of expression (Table 6—supplement; FIG. 5).

Therefore the RRR shares with RCC two qualitative gene expression signatures: a concordant and a discordant. The genes in the two signatures are significantly subject to regulation by similar pathways as well as significantly unique pathways (p<0.05). The probability of being able to observe these concordant (77% RRR/RCC) and discordant (23% RRR/RCC) genes merely through chance would be extremely low if RRR and RCC phenotype were unrelated (p-value 2.2e-16, binomial test).

### The Biological Basis of Concordantly and Discordantly Expressed Genes in RRR and RCC

In the search for the biological basis of the concordant and discordant groups, we analyzed these genes using the Gene Ontology consortium ontologies (GO), (Fisher Exact p<0.05), (http://www.geneontology.org). This method revealed that the concordant genes were significantly involved in such molecular functions as immunoglobulin binding, ECM structural constituent conferring tensile strength activity, structural constituents of ribosomes, RNA binding, cell adhesion (mainly by RRR up-regulated genes), and selenium binding (mainly by RRR down-regulated genes). The over all concordant gene expression was up-regulated in cellular components that included the cytosolic ribosome the proteasome core complex, collagen, the small ribosomal subunit, and the microfibril. The biological processes with an overall concordant gene up-regulated expression were DNA replication initiation, ribosome biogenesis, macromolecule biosynthesis, cytoplasm organization and biogenesis, cell death, cell adhesion, immune response, and protein metabolism. Process with mainly down-regulated concordant genes included phenylalanine metabolism and catabolism, tyrosine metabolism, and cell ion homeostasis. Other significant processes affected included regulation of translation, posttranslational membrane biomarkering, ER organization and biogenesis, and cell growth and/or maintenance (Table 6,4-supplement).

On the other hand, the discordant genes were significantly (Fisher Exact p<0.05) found in molecular functions as insulin-like growth factor binding, organic cation transporter activity, and heparin binding. The discordant genes were significant in the cellular component of extracellular space and were significantly associated with the molecular processes of one-carbon compound metabolism, angiogenesis, regulation of cell growth, actin cytoskeleton organization and biogenesis, actin filament-based processes, enzyme-linked receptor protein signaling, organelle organization and biogenesis, and organogenesis (Table 6,4-supplement).

Following this analysis, we then cross-compared gene ontologies (Fisher Exact p<0.05), among the concordant group, the discordant group, and the group continuously involved in all three phases of RRR, which we correlated above with Sutton's four-phase model of RRR (Sutton T A et al 2002).

During the early phase of RRR the gene category of DNA replication initiation was significantly present and consisted of five up-regulated genes. These five genes belong to the family of minichromosome maintenance proteins (MCM) and included MCM2, MCM3, MCM4, MCM5, and MCM7. With the exception of MCM5, these genes have been reported to be up-regulated concordantly in RCC pathogenesis (Table 1—supplement, Table 6).

The discordant genes significantly shared the ontology of growth factor binding with the early phase, and the ontology of extracellular space with the late phase (Table 5-supplement). During the early phase, discordant genes in the “growth factor binding” ontology were associated with the IGF pathway. Both connective tissue growth factor (CTGF/IGFBP8) and cysteine-rich protein 61 (CYR61) were up-regulated in RRR, while insulin-like growth factor binding proteins 1 and 3 (IGFBP1 and 3) were down-regulated in RRR. The discordant genes belonging to the late phase ontology of extracellular space that were up-regulated in RRR and included apolipoprotein E (APOE), connective tissue growth factor (CTGF), decorin (DCN), glypican 3 (GPC3), matrix metalloproteinase 2 (MMP2), plasminogen activator, tissue (PLAT), and thrombospondin 1 (THBS1). In contrast, growth arrest and D-damage-inducible 45 gamma (GADD45G) was down-regulated in RRR. Except for GADD45G, the genes of this group shared a pattern of expression with trends 5 and 6, which were also up-regulated in RRR at two weeks after the initial trauma (Table 6).

Among its 46-gene complement, trend 11 contains 4 concordant (p>0.05) and 9 significant discordant genes (p<0.0003). All of these genes proved to be down-regulated in RRR and included superoxide dismutase 2 (SOD2), cytochrome c oxidase subunit VIc (COX6C), kinesin family member 21A (KIF21A), kallikrein 1 (KLK1), heat shock 105 kDa/110 kDa protein 1 (HSPH1), carcinoembryonic antigen-related cell adhesion molecule 1 (CEACAM1), methionine adenosyltransferase II, alpha (MAT2A), PCTAIRE protein kinase 3 (PCTK3), and serine hydroxymethyltransferase 2 (SHMT2). The last four genes were also regulated by the VHL pathway (Table 6).

We then extended the gene ontologies (Fisher Exact p<0.05) to a cross-comparison with the following groups: total gene-expression data, the sub-sets for early and/or late RRR, expression trends, pathways such as IGF, concordance and discordance with RCC, oncogenes, tumor suppressors, and metastasis (FIG. 4—supplement, 5, 6, 7).

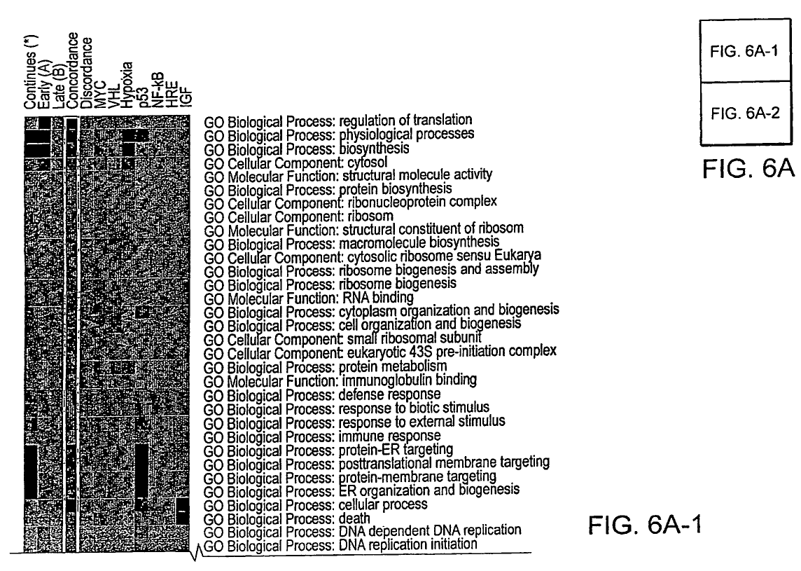

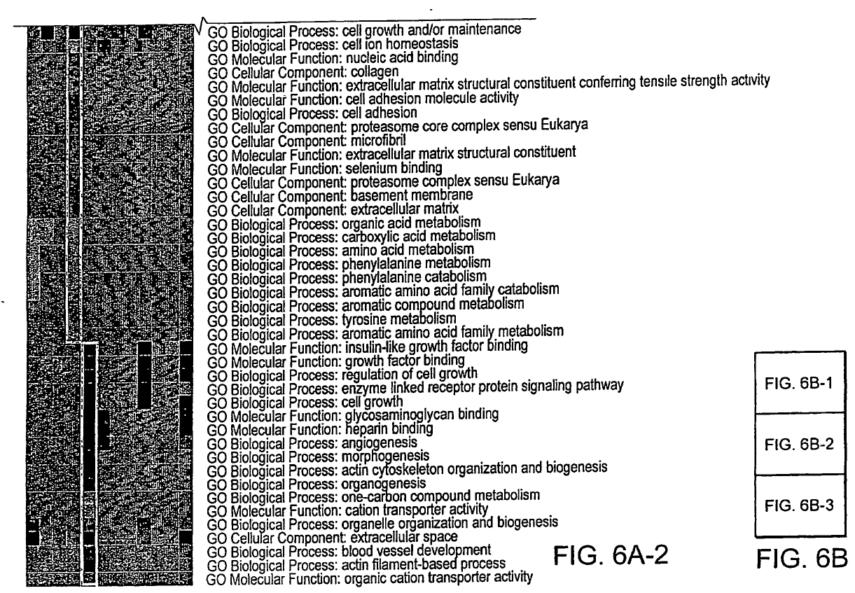

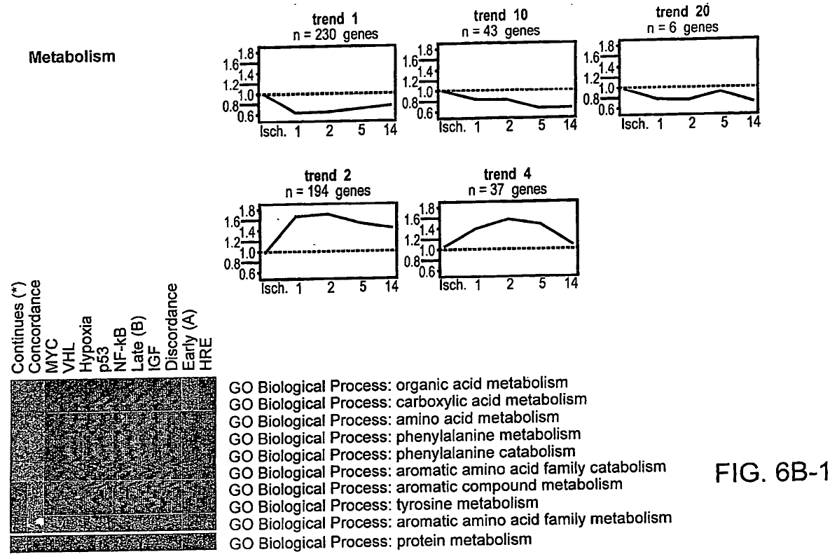

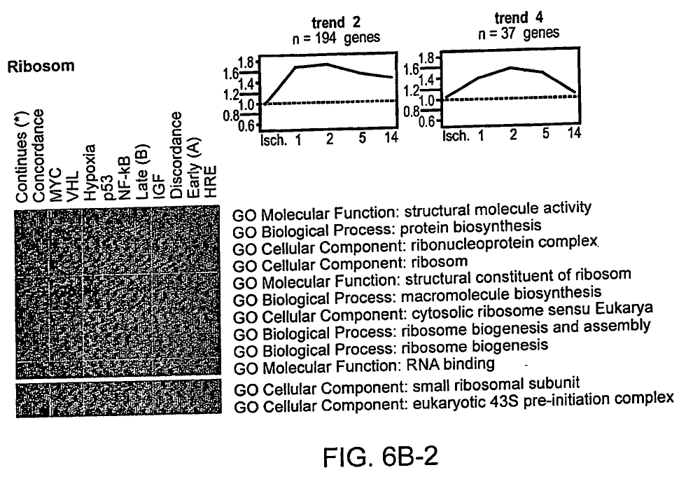

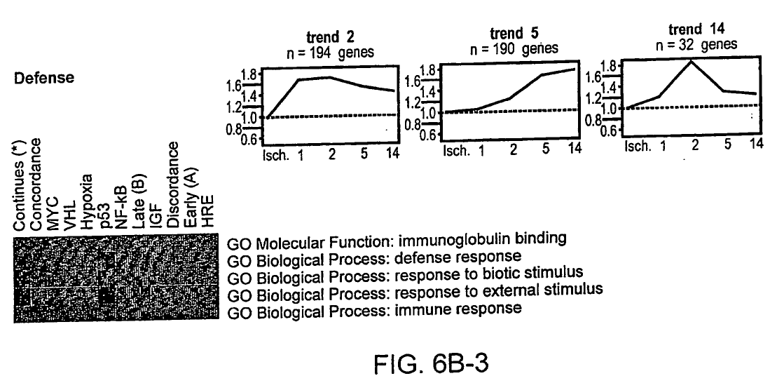

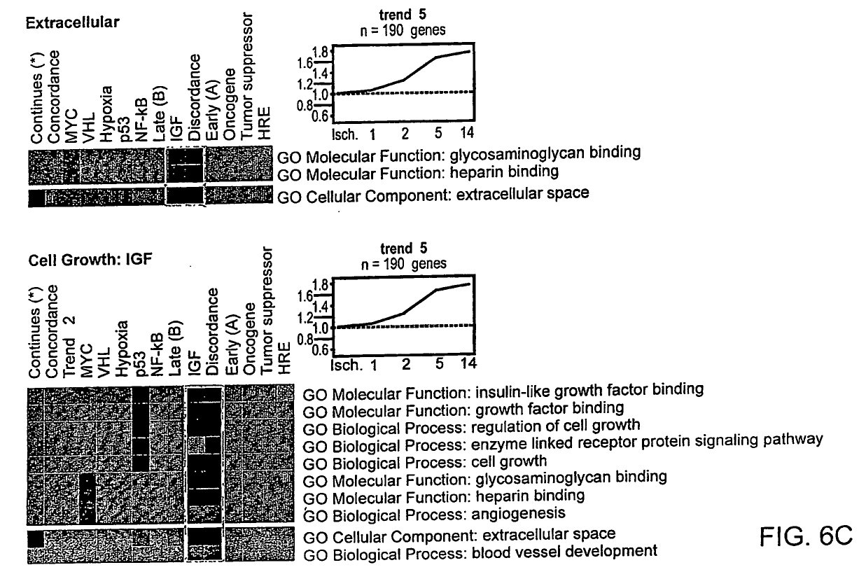

The concordant genes and trend 2 (up-regulated in the early RRR and moderately down regulated at the late RRR) corresponded primarily with ontologies of ribosome and defense (FIG. 6). Possibly, a sub-set of this pattern was also involved in the Hypoxia and VHL pathways, senescence, and trend 4, which was up-regulated during early RRR, but returning to normal expression levels at two weeks of RRR (FIG. 6). P53 and NF-kB were regulating ontologies in defense/immune responses, death process and ER genes (FIG. 6).

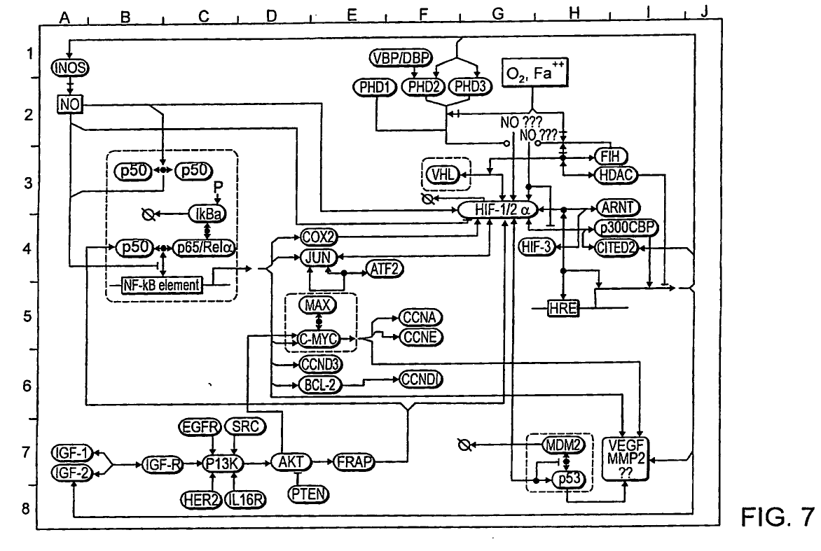

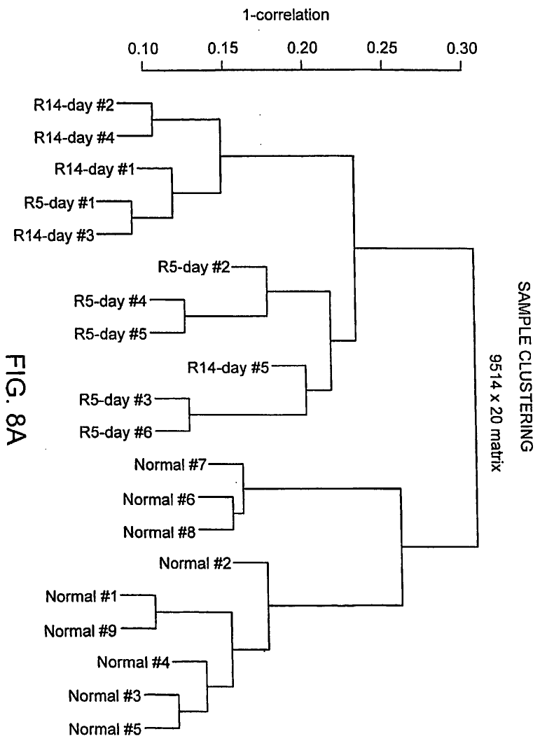

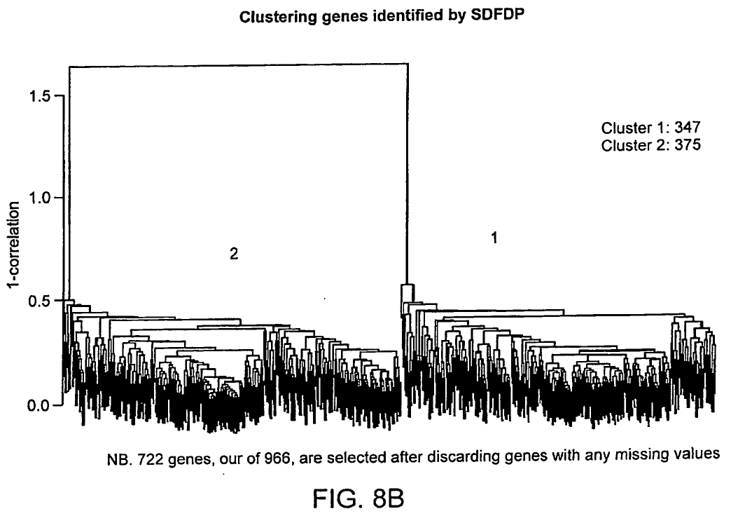

The ontologies involved in the IGF pathway were also present in the genes discordantly expressed between RCC and RRR. These included such processes as cell growth and angiogenesis and functions as growth factor binding, enzymatic reactions, glycosaminoglycan binding, and heparin binding. Finally, certain cellular components, including ECM, were co-represented in both the IGF pathway and the RCC discordant gene subset. Because both the IGF pathway and the discordant gene subset share genes to a significant degree, we suggest that the IGF pathway plays a functional role in RRR and RCC (FIGS. 5, 7).

We also catalogued the discordant genes on a non-probabilistic, gene-by-gene basis (Table 7). Most of the changed genes in the discordant group belong to subgroups that are in important in maintaining cell structure, gene expression, ECM function, angiogenesis, DNA repair, catabolism, mitochondrial functions, motility, catalytic activity, stress signals, external signals, ubiquitination, immunity, oxidation, metastasis, migration, and adhesion. Similarly to the results of our previous analysis (Table 3), genes regulated discordantly when comparing normal RRR and RCC, proved or suggested to be regulated by the IGF, VHL-HIF, hypoxia, C-MYC, p53, or NF-kB pathways. Moreover, some of these genes are known to play roles in pathways involved in senescence, tumor suppression, or oncogenesis.

### Characterization of the Histopathology of RRR

Early histopathologic features of ischemic injury induced by 50 minutes of vascular clump were readily evident in the kidney within the first 12 hours of reperfusion and were monitored at 1, 2, 5, 7 and 14 days. As expected (Suparvekin S. et al 2003), we observed apoptotic cells in the outer medulla within 12 hours of reperfusion, which became more abundant over the first 24 hours following initial injury (data not shown). At one day after the ischemic event, more than half of cortical tubules (FIG. 2C) showed some degree of staining for glucose transporter-1 (Glut-1/SLC2A1), which is regulated by the transcription factor hypoxia-inducible factor 1 (HIF1). Up-regulation of HIF1 provides tissue protection from ischemic damage during the early regeneration pattern (Matsumoto M. et al 2003). At 2 days, we observed by hematoxylin and eosin (H&E) staining an acute tubular necrosis in which about half of the tubules showed necrosis with loss of epithelium; the remaining tubules showed cells with reactive nuclear changes (hyperchromasia, prominent nucleoli) (FIGS. 2A, 2B). At 2 days, the necrotic-apoptotic events were accompanied by positive tubules staining with the proliferation marker MiB-1 (FIG. 2B). At two weeks, most tubules showed a normal appearance with only rare examples showing degenerative or regenerative changes (FIG. 2B). Thus, the histological evidence reported here supports the accepted process of renal injury, regeneration, and recovery (Sutton T A et al 2002). Damaged renal tissue is first characterized by regenerating tubules in which necrotic cells are accompanied by replicating cells; at two weeks, most tubules have recovered and regained their normal appearance

### Characterization of Differential Gene Expression as a Consequence of Renal RRR: Defined Patterns of Early, Late and Continuous Tissue Regeneration

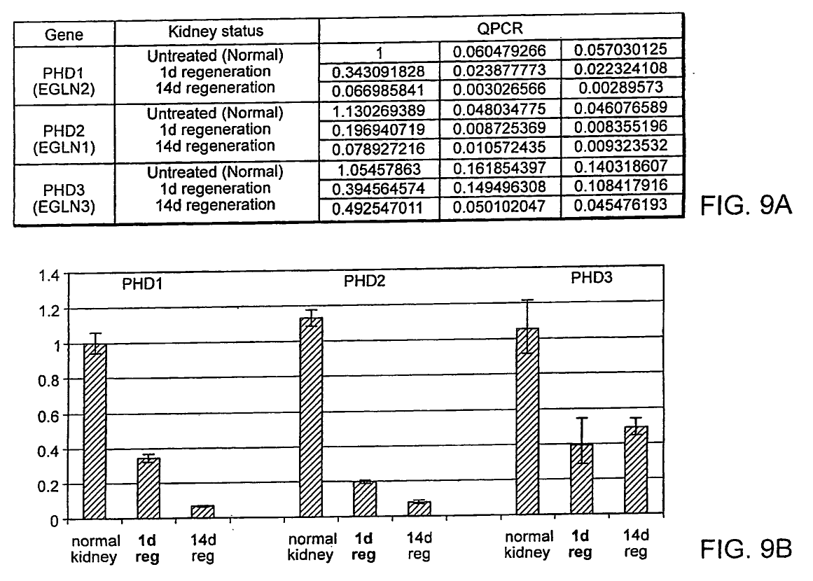

Employing cDNA microarray analysis of 9,646 genes, we were able to compare the changes in the global pattern of gene expression of normal (day 0), ischemic (50 minutes) and reperfused (at 1, 2, 5 and 14 days) kidney issue. A differential expression pattern was observed for a group of 1,350 gene spots, corresponding to 1,325 genes (P-value ≦0.05). This differential pattern clustered into a dendrogran consisting of four main branches (FIGS. 3, 9). The first branch included the normal and ischemic kidney tissue; the second branch included differentially expressed genes accompanying regenerative processes taking place continuously throughout the two-week period (FIG. 3 marked as asterisk); the third branch was of genes differentially expressed during early regenerative processes taking place during the first two days following reperfusion (FIG. 3 marked as A); and finally, the fourth branch included genes differentially expressed late, at 5 and 14 days after reperfusion (FIG. 3 marked as B).

The differential expression of each gene was averaged and calculated as relative to the same gene expressed in normal and ischemic kidney tissues. All the repetitive samples clustered together, illustrating the reproducibility of the animal model and supporting the reliability of the array methodologies employed. Therefore, relative to the normal kidney, we identified three patterns of differentially expressed genes during RRR: continuous, early and late.

Of the 1,325 RRR genes that were differentially expressed from normal kidney during the first two weeks, 323 genes were in the continuously pattern (189 genes up-regulated and 134 genes down-regulated); in the early pattern of RRR, 629 genes were differentially expressed (336 genes up-regulated and 293 genes down-regulated) and in the late pattern of RRR, 373 genes were differentially expressed (227 genes were up-regulated and 96 genes down-regulated), (Table 1). Table 1 summarizes the data related to the numbers of genes that were differentially expressed and are therefore of potential functional importance in general biological processes involved in RRR. A complete listing of all genes is given in the supplemented Table 9.

The RRR differential gene expression as compared to normal kidney was further clustered to identify different temporal trends over the two week period. We statistically identified 27 trends that are described in details in the supplemental material. The 6 major trends are represented in FIG. 4. The up-regulated trends (FIG. 4A-C) consists of trend 5 (FIG. 4A) that represents 190 genes that were early up-regulated and remained up-regulated on the 14^{th} day of RRR and trends 2 and 4 (FIG. 4B-C) are of pattern seen for 194 and 37 genes, respectively, that were up-regulated at the early pattern (days 1 and 2) and reduced towards normal levels at the late pattern (days 5 and 14).

The down-regulated trends (FIG. 4D-E) consists of trend 1 (FIG. 4D) represents the major patterns of genes that were down-regulated during RRR and partially returned towards normal levels, by day 14, (n=270). Similarly, trends 16 and 11 (FIGS. 4E, 4F) contain 87 and 11 genes, respectively, that were down-regulated at days 1 and 2, but were getting back to normal levels on day 5. Other temporal trends are discerned statistically, but follow similar tendency as the representative trends shown, which contain the majority of the differentially expressed genes.

Identification of Specific Functional Gene-Clusters by Ontology Analysis, Probabilistic Functional Genomics, and Cross-Comparison with the Pathway Literature

Similarities and Differences Between RRR and RCC

Previous reports suggested that RRR and or RCC subject to regulation by hypoxia and a number of pathways as VHL, HIF, IGF, Myc, p53 and NF-kB (Elson D. A. et al., 2000, Maxwell P H. 2004, Schips L et al 2004, Hammerman M R 1999, Yamaguchi S et al 2003, Koshiji M et al 2004, Schmid T et al 2004, Qi H and Ohh M2004, Cao C C et al 2004). We therefore tested if biomarker genes of these pathways or their regulators were significantly found in the 285 concordantly expressed genes. In both RRR and RCC the concordant genes significantly (p<0.05) included genes regulated by hypoxia and pathways including VHL, Myc, p53 and NF-kB. HIF and IGF pathway genes were also evident among the concordant genes but with association significance of p>0.05 (Table 4).

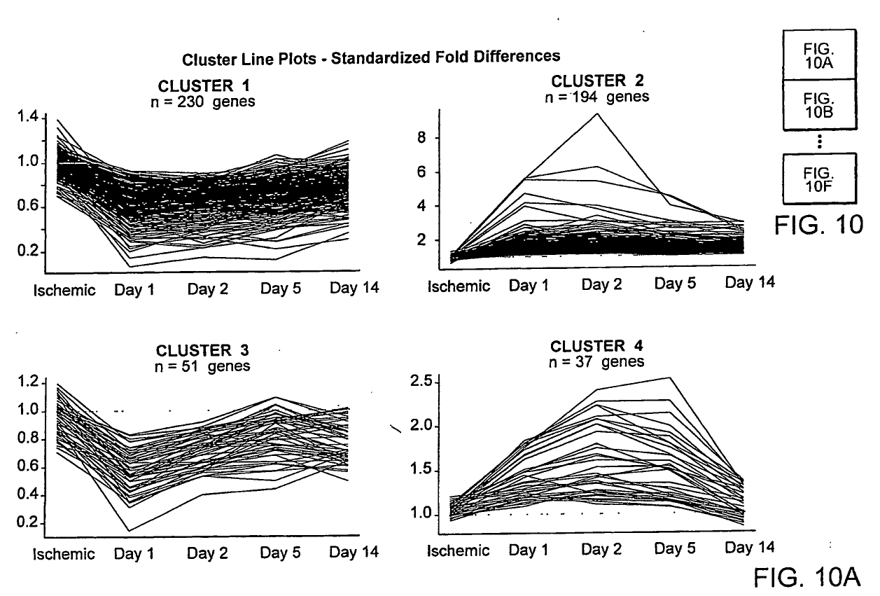

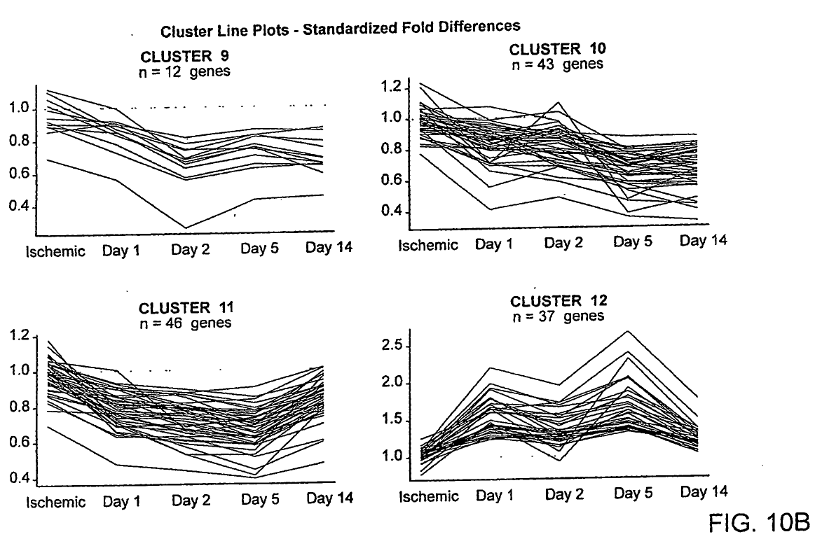

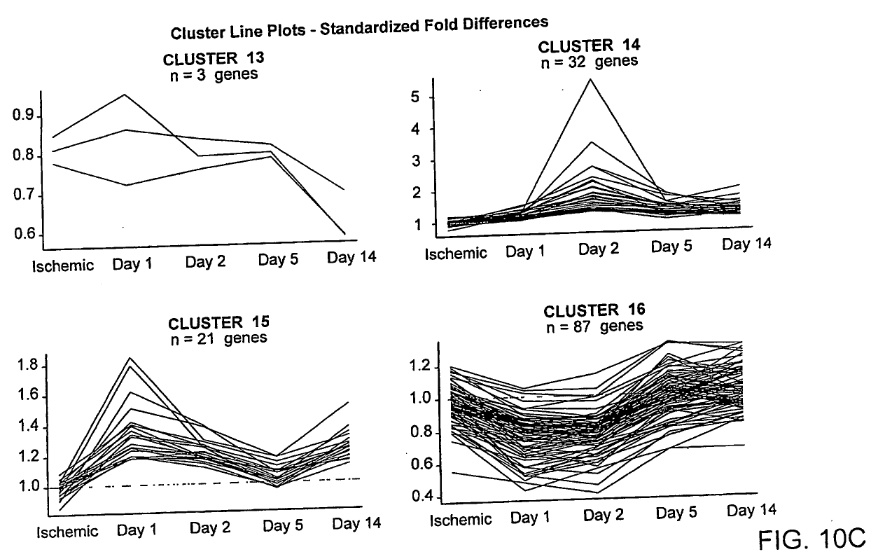

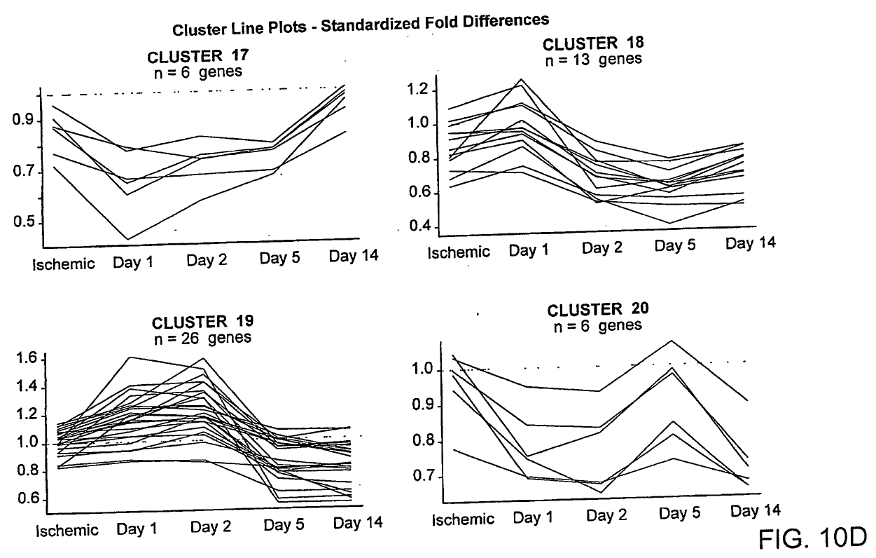

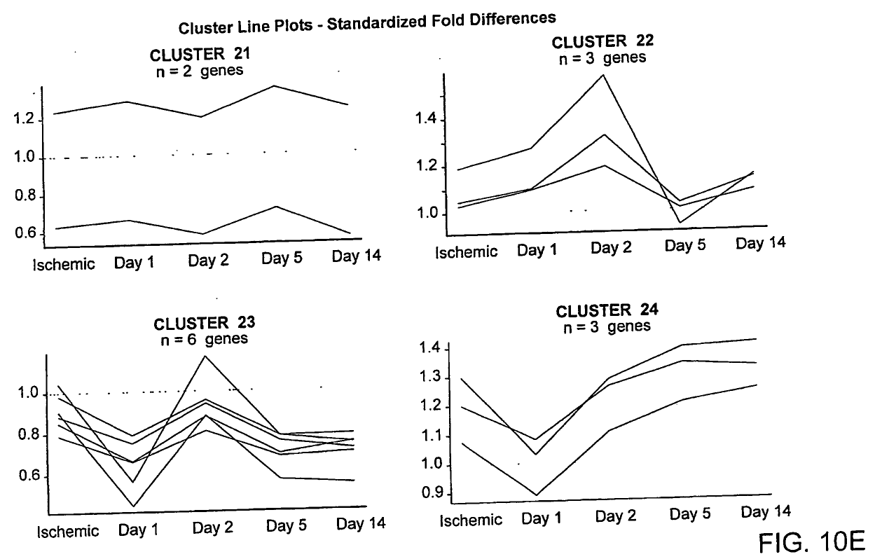

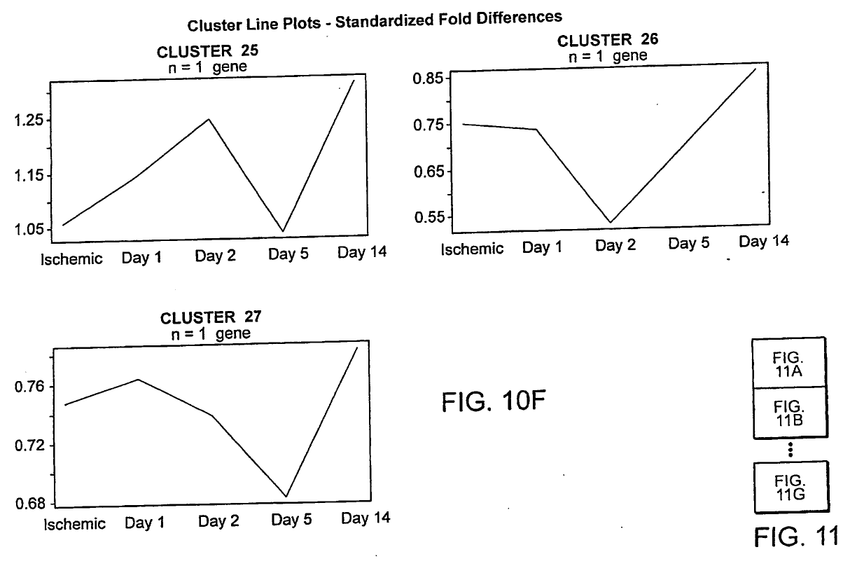

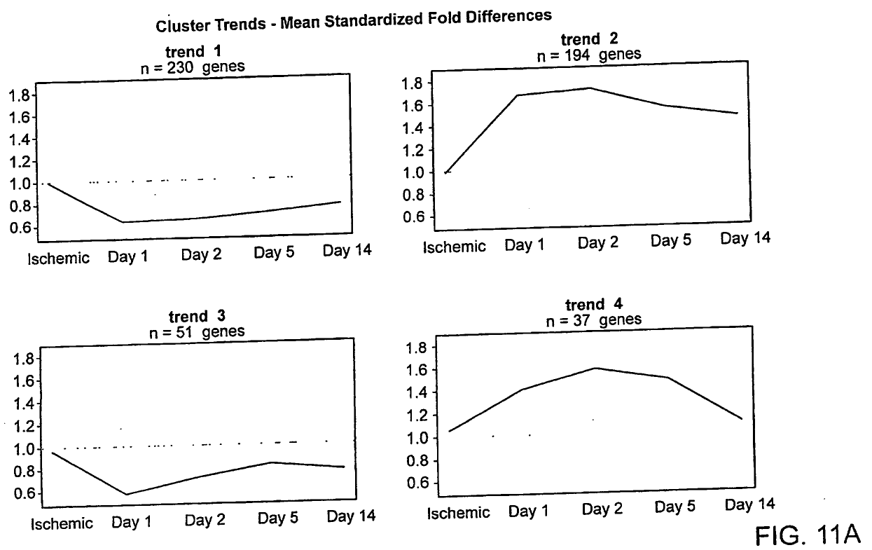

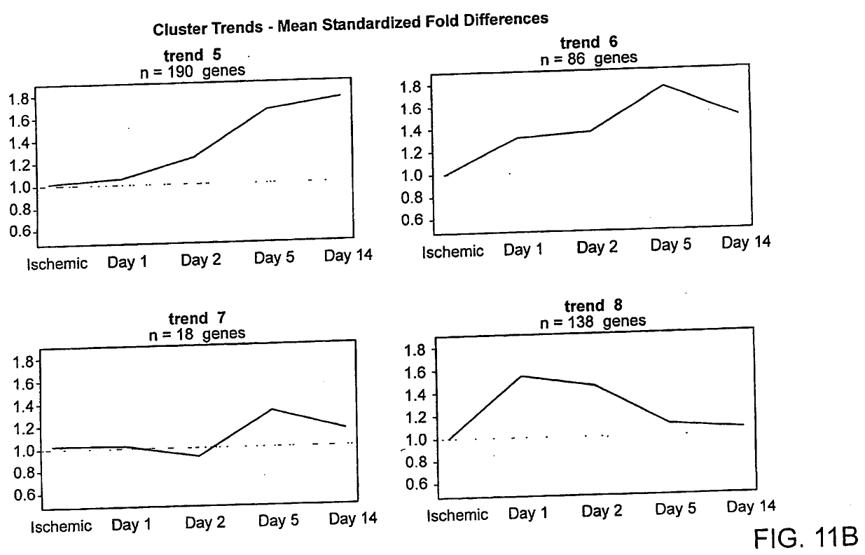

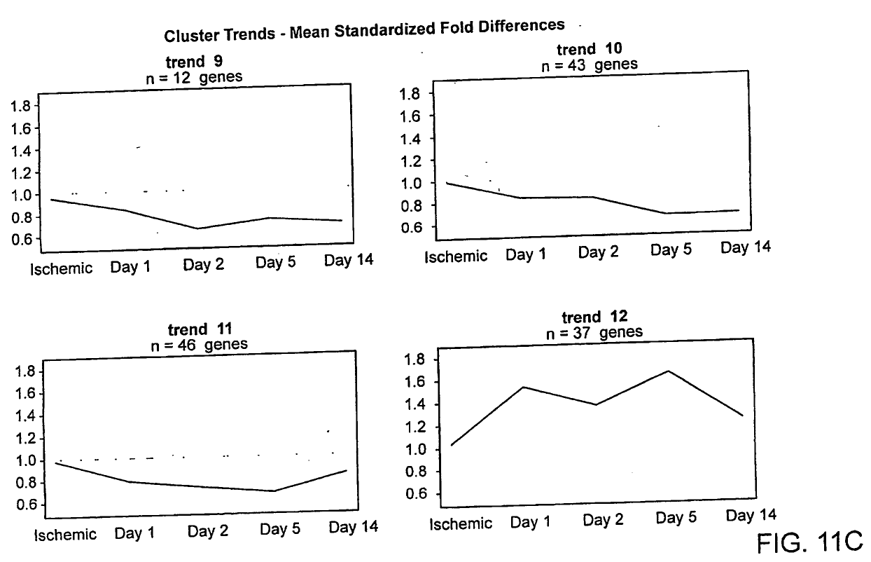

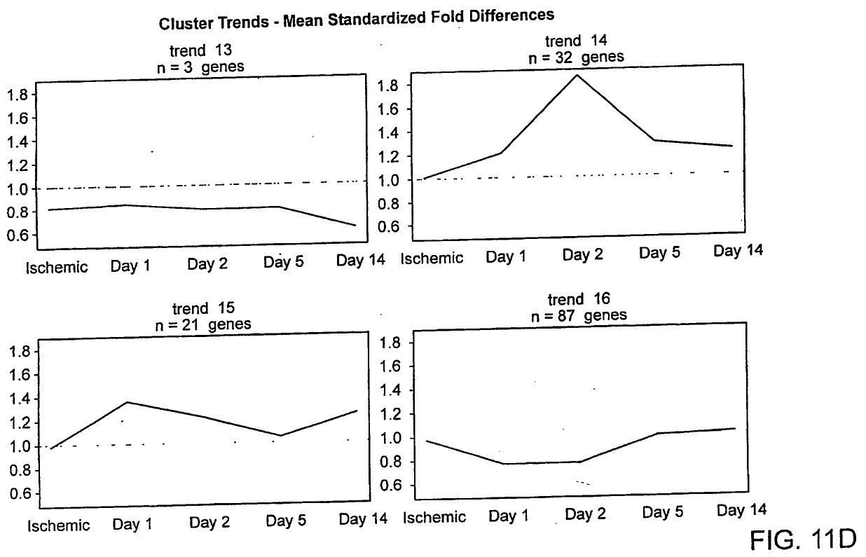

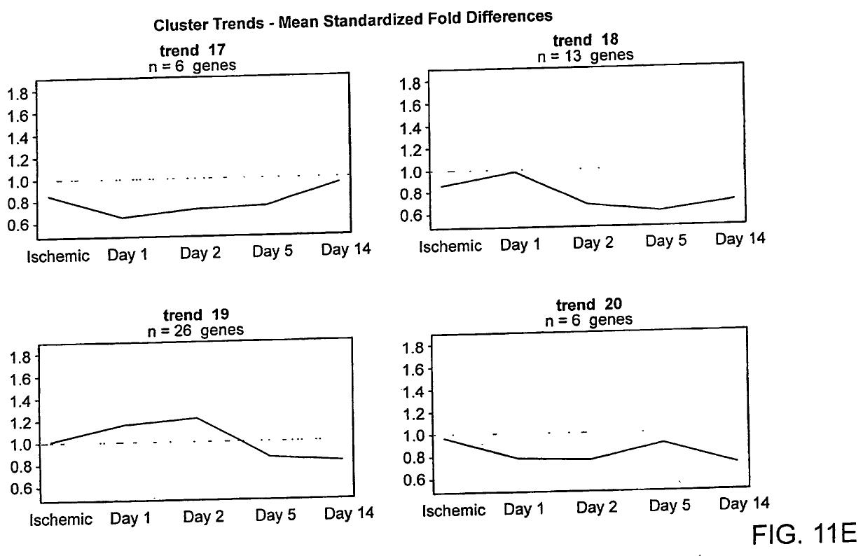

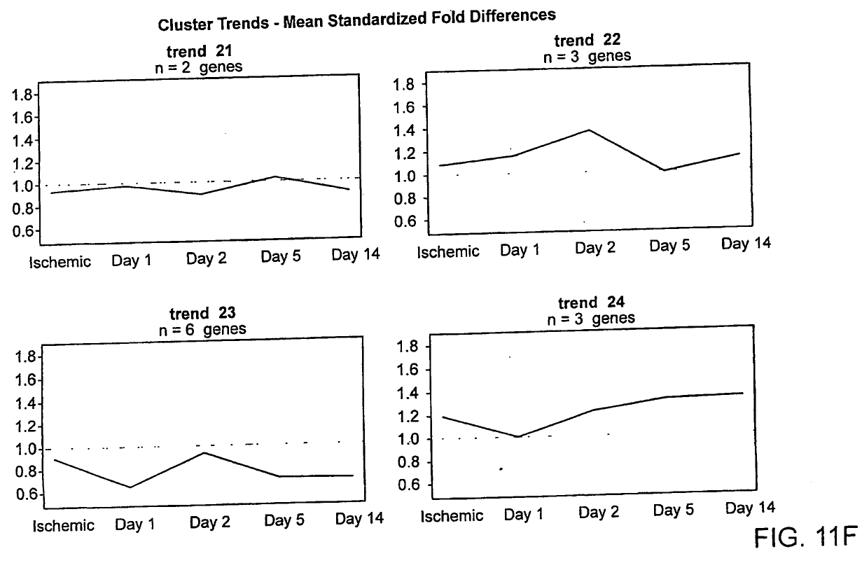

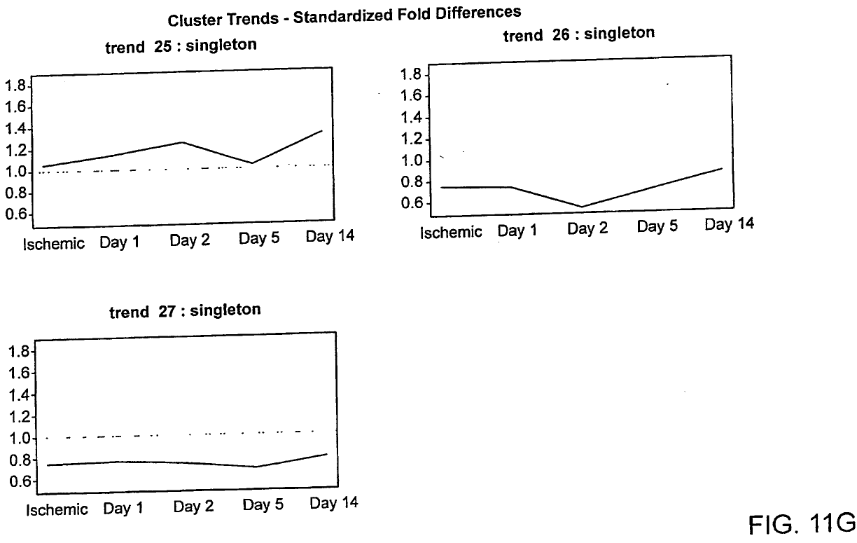

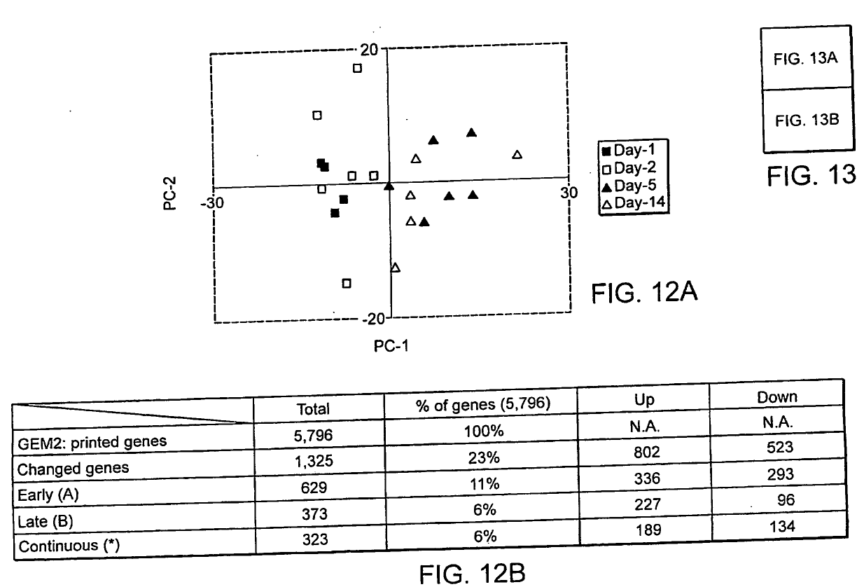

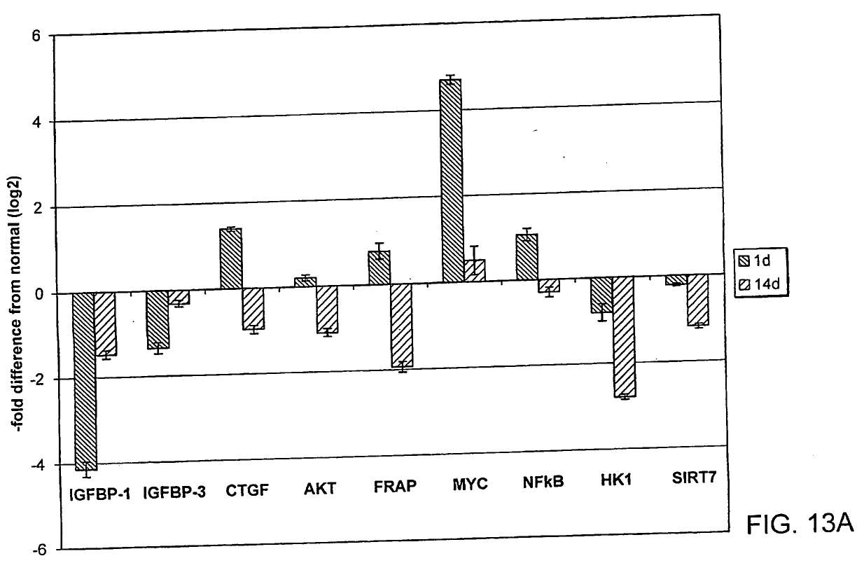

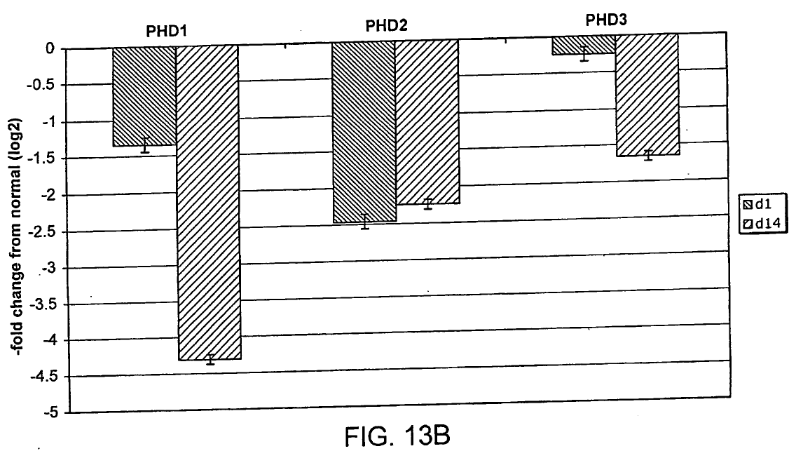

The concordant genes were significantly (p<0.05) expressed in six of the temporal patterns/trends of gene expression and included the up-regulated trends: 2, 4, 6, 14 and the down-regulated trends 1 and 16 (FIG. 4 and supplemented FIG. 10 and Table 12). Further, trends 1, 4, 6 and 14 were significant to the concordant genes and not to the discordant one (the temporal patterns/trends of gene expression are described in the Characterization of differential gene expression as a consequence of renal Ischemia) (FIG. 4 and supplemented FIG. 10 and Table 12).

The remainder of the 361 genes, 81 genes (23%), were discordantly expressed during RRR as compared to RCC. Of these 83 discordant genes, 30 genes were in RRR up-regulated and in RCC down-regulated (i.e. FHIT, MMP2, APOE, CTGF, DCN, PLAT, THBS1, WSB1, SLC1A1, SMC1L1), (tables 7, 9). The rest of the 53 genes were down-regulated in RRR and up-regulated in RCC (i.e. IGFBP1, IGFBP1, PHD2/EGLN1, Nulp1 (KIAA1049), VEGFA, KDR/VEGFR2, ACOX1, CPT1A, HK1, SLC16A7/MCT2, RRM1, ENPP2, COX6C, TOP3B, PAPOLA/PAP and SLC22A1), (tables 7, 9). Of significance (p<0.05) were genes in the pathways of VHL, hypoxia, HIF1a (HRE), IGF, and p53. HIF and IGF pathways are significantly distinct to the discordant genes and not for the concordant genes. On the other hand, genes in the NP-kB pathway were significant for the concordant genes, but only evident among the discordant genes, with association significance of p>0.05 (Table 4).

Three temporal patterns/trends of gene expression, down-regulated trends 2, 11, and the up-regulated trend 16, significantly included discordant genes (p<0.05). Trend 11 was significantly distinct to the discordant genes and not the concordant genes. Trend 11 trend encompassed 46 down-regulated genes (9 of which were discordantly expressed) active from the first day until the fifth day of RRR, when they began to return to normal levels of expression (FIG. 4 and supplemented FIG. 10 and Table 12).

Therefore the RRR shares with RCC two qualitative gene expression signatures: a concordant and a discordant. The genes in the two signatures are significantly subject to regulation by similar pathways as well as significantly distinct pathways (p<0.05). Finally, the probability of being able to observe these concordant (77% RRR/RCC) and discordant (23% RRR/RCC) genes merely through chance would be extremely low if RRR and RCC phenotype were unrelated (p-value 2.2e-16, binomial test) (Table 4).

### The Biological Basis of Concordantly and Discordantly Expressed Genes in RRR and RCC

In the search for the biological basis of the concordant and discordant groups, we analyzed these genes using the Gene Ontology consortium ontologies (GO), (Fisher Exact p<0.05), (http://www.geneontology.org). This method revealed that the concordant genes were significantly involved in such molecular functions as immunoglobulin binding, ECM structural constituent conferring tensile strength activity, structural constituents of ribosomes, RNA binding, cell adhesion (mainly by RRR up-regulated genes), and selenium binding (mainly by RRR down-regulated genes). The overall concordant gene expression was up-regulated in cellular components that included the cytosolic ribosome, the proteasome core complex, collagen, the small ribosomal subunit, and the microfibril. The biological processes with an overall concordant gene up-regulated expression were DNA replication initiation, ribosome biogenesis, macromolecule biosynthesis, cytoplasm organization and biogenesis, cell death, cell adhesion, immune response, and protein metabolism. Process with mainly down-regulated concordant genes included phenylalanine metabolism and catabolism, tyrosine metabolism, and cell ion homeostasis. Other significant processes affected included regulation of translation, posttranslational membrane biomarkering, ER organization and biogenesis, and cell growth and/or maintenance (Table 5).

On the other hand, the discordant genes were significantly (Fisher Exact p<0.05) found in molecular functions as insulin-like growth factor binding, organic cation transporter activity, and heparin binding. The discordant genes were significant in the cellular component of extracellular space and were significantly associated with the molecular processes of one-carbon compound metabolism, angiogenesis, regulation of cell growth, actin cytoskeleton organization and biogenesis, actin filament-based processes, enzyme-linked receptor protein signaling, organelle organization and biogenesis, and organogenesis (Table 5).

Following this analysis, we then cross-compared gene ontologies (Fisher Exact p<0.05), among the concordant group, the discordant group, and the group continuously involved in all three patterns of RRR, which we correlated above with Sutton's four-pattern model of RRR (Sutton T A et al 2002).

During the early pattern of RRR the gene category of DNA replication initiation was significantly and distinctly present in the concordant genes and consisted of five up-regulated genes. These five genes belong to the family of minichromosome maintenance proteins (MCM) and included MCM2, MCM3, MCM4, MCM5, and MCM7. With the exception of MCM5, these genes have been reported to be up-regulated concordantly in RCC pathogenesis (Tables 6 and 9).

The discordant genes significantly shared the ontology of growth factor binding with the early pattern, and the ontology of extracellular space with the late pattern (Tables 6 and 9).

During the early pattern, discordant genes in the “growth factor binding” ontology were associated with the IGF pathway. Both connective tissue growth factor (CTGF/IGFBP8) and cysteine-rich protein 61 (CYR61) were up-regulated in RRR, while insulin-like growth factor binding proteins 1 and 3 (IGFBP1 and 3) were down-regulated in RRR. The discordant genes belonging to the late pattern ontology of extracellular space that were up-regulated in RRR and included apolipoprotein E (APOE), connective tissue growth factor (CTGF), decorin (DCN), glypican 3 (GPC3) plasminogen activator, tissue (PLAT), and thrombospondin 1 (THBS1). In contrast, growth arrest and D-damage-inducible 45 gamma (GADD45G) was down-regulated in RRR Except for GADD45G, the genes of this group shared a pattern of expression with trends 5 and 6, which were also up-regulated in RRR at two weeks after the initial trauma (Tables 6 and 9).

Among its 46-gene complement, trend 11 contains 4 concordant (p>0.05) and 9 significant discordant genes (p<0.0003). All of these genes proved to be down-regulated in RRR and included superoxide dismutase 2 (SOD2), cytochrome c oxidase subunit VIc (COX6C), kinesin family member 21A (KIF21A), kallikrein 1 (KLK1), heat shock 105 kDa/110 kDa protein 1 (HSPH1), carcinoembryonic antigen-related cell adhesion molecule 1 (CEACAM1), methionine adenosyltransferase II, alpha (MAT2A), PCTAIRE protein kinase 3 (PCTK3), and serine hydroxymethyltransferase 2 (SHMT2). The last four genes were also regulated by the VHL pathway (FIG. 4, Table 5).

We then extended the gene ontologies (Fisher Exact p<0.05) to a cross-comparison with the following groups: total gene-expression data, the sub-sets for early and/or late RRR, expression trends, pathways such as IGF, concordance and discordance with RCC (FIGS. 6 A-C, Tables 4, 5).

The concordant genes and trend 2 (up-regulated in the early RRR and moderately down regulated at the late RRR) corresponded primarily with ontologies of ribosome and defense (FIG. 6 A-B). Possibly, a sub-set of this pattern was also involved in the Hypoxia and VHL pathways, and trend 4, which was up-regulated during early RRR, but returning to normal expression levels at two weeks of RRR (FIG. 6 A-B). P53 and NF-kB were regulating ontologies in defense/immune responses, death process and ER genes (FIG. 6 A-B).

The ontologies involved in the IGF pathway were also present in the genes discordantly expressed between RCC and RRR. These included such processes as cell growth and angiogenesis and functions as growth factor binding, enzymatic reactions, glycosaminoglycan binding, and heparin binding. Finally, certain cellular components, including ECM, were co-represented in both the IGF pathway and the RCC discordant gene subset. Because both the IGF pathway and the discordant gene subset share genes to a significant degree, we suggest that the IGF pathway plays a functional role in RRR and RCC (FIGS. 6 A, C).

Even this comprehensive probabilistic analysis may fail to capture many key aspects of discordant gene function. To mitigate this possibility, we also catalogued the discordant genes on a non-probabilistic, gene-by-gene basis (Table 7). Most of the changed genes in the discordant group belong to subgroups that are in important in maintaining cell structure, gene expression, ECM function, angiogenesis, DNA repair, catabolism, mitochondrial functions, motility, catalytic activity, stress signals, external signals, ubiquitination, immunity, oxidation, metastasis, migration, and adhesion. Similarly to the results of our previous analysis (Table 4), genes regulated discordantly when comparing normal RRR and RCC, proved or suggested to be regulated by the IGF, VHL-HIF, hypoxia, C-MYC, p53, or NF-kB pathways. Moreover, some of these genes are known to play roles in pathways involved in senescence, tumor suppression, or oncogenesis.

We next utilized probabilistic functional genomics to complement the comparison of the concordantly and discordantly expressed genes between RRR and RCC (the full and comprehensive probabilistic functional genomics analysis is currently under preparation for publication). Of great interest is the enrichment for the ARNT (HIF-1b) homodimer element in the promoter regions of the concordat genes (loading of −4.169418). 21 concordantly expressed genes were up-regulated and 9 genes down regulated and included continuously, early and late expressed genes (Table 8). Also, 6 discordantly expressed genes were suggested to have the ARNT homodimer element, one of which is Egln1.

We pursued a cross-comparative approach in analyzing gene expression patterns and regulatory mechanisms implicated in wound healing and/or RCC pathogenesis. We observed a high degree of concordance among the genes differentially expressed in both RRR and RCC. However, we also observed a discordant differential gene expression that differentiated the RRR and RCC and might be specific to malignant transformation. Further, we have identified gene expression programs of pathways, functions, and cellular locations that appear to play a multifaceted role in wound healing and/or carcinogenesis.

### Renal Ischemia—Reperfusion as a Wound Healing Model

To induce tissue regeneration in normal mouse kidney, we chose to use a unilateral renal ischemia model. The predominant consequences of renal injury in this model include proximal tubule necrosis, as well as apoptosis in a minority of the cells. The reversal of these changes coincides with the reestablishment of the normal renal epithelial barrier as new cells reline the denuded tubules (Price, P. M. et al., 2003). Wound healing is a complex, but orderly phenomenon involving a number of principle processes: induction of acute inflammatory processes by the initial injury; regeneration of parenchymal cells; migration and proliferation of parenchymal and connective tissue cells; synthesis of ECM proteins; remodeling of connective tissue and parenchymal components; and finally, collagenization and acquisition of wound tensile strength (Cotran, R. S. et al., 1999). Regions of hypoxia are common in healing wounds, and the state of hypoxia alters the activity of selected transcription factors, including HIF-1a, HIF-2a, JNK, NF-kB, c-MYC, IGF, and p53. These transcriptional activations result in increased expression of growth factors, growth factor receptors, and angiogenic factors (Tables 2, 3, 9), (Elson D. A. et al., 2000, Maxwell P H. 2004, Schips L et al 2004, Hammerman M R 1999, Yamaguchi S et al 2003, Koshiji M et al 2004, Schmid T et al 2004, Qi H and Ohh M 2004, Cao C C et al 2004).

### Patterns of Differentially Expressed Genes in RRR

Using global gene expression analysis, we have demonstrated that RRR characterized by three general patterns of differentially expressed genes referred to as “early,” “late,” and “continuous,” which includes early and late events (FIG. 3, Table 1).

In terms of Sutton's renal RRR model (Sutton T A et al 2002)—initiation, extension, maintenance, and repair—the “continuous” (early and late) pattern we have defined encompasses gene functions relating to all four patterns. The “early” pattern subsumes functions related to initiation, extension, and early maintenance, while our “late” pattern of RRR includes maintenance as well as recovery. Our data supports a model of ischemic RRR as a complex, but orderly continuum composed of overlapping patterns that continuously up-regulate the immune response and down-regulate oxidoreductase activity. Gene functions relating to dedifferentiation, migration, proliferation, redifferentiation, and repolarization are associated with the Maintenance and repair patterns in. Sutton's model. Refining this, we have observed that during early RRR, the regulated genes are involved in cell proliferation and only during late RRR do genes implicated in redifferentiation become differentially expressed (Table 2).

### Normal RRR Processes are Found in RCC

Through the comparative analysis of global gene expression patterns characteristic of RRR and RCC, we have identified a total of 361 genes implicated in one or both processes, as well as global regulatory patterns that are shared concordantly (278 genes) or discordantly (83 genes) between renal wound healing (RRR) and carcinoma (RCC). The probability of observing such an ensemble of concordant and discordant genetic activity by chance would be highly unlikely if RRR and RCC phenotypes were unrelated (p-value 2.2e-16, binomial test) (FIG. 5, Table 4).

Concordant genes comprised the majority (77%) of the 361 genes we identified; most of the genes in this group were related to processes involved in renal cell maintenance, including metabolic functioning, DNA replication, cellular defense, immune response and cell death (Table 5).

DNA replication is an essential step in both normal and transformed dividing cell. We found that four members of the highly conserved mini-chromosome maintenance (MCM2, 3, 4 and 7) protein family are concordantly up-regulated during the early pattern of RRR and in RCC (p<0.05). A fifth member, MCM5 is also up-regulated during the early pattern of RRR, but the expression in RCC needs to be tested. The complex formed by MCM proteins is a key component of the pre-replication complex and may be involved in the formation of replication forks and the recruitment of other DNA-replication-related proteins.

The concordantly expressed genes also include 167 genes that retained the normal renal cell program of apoptosis (Table 5) and may thus indicate that the apoptotic mechanism is partially maintained in RCC. Furthermore, we observed that the anti-apoptotic and anti-inflammatory gene heme oxygenase-1 (HO-1/HMOX1) is up-regulated in both RRR and RCC; thus, it is possible, perhaps probable, that the up-regulated gene contributes to cytoprotection during each process (Goodman A. I. et al., 1997, Adachi S et al., 2004).

Our probabilistic functional genomics comparison of the concordantly with the discordantly expressed genes between RRR and RCC, suggests an enrichment for the binding element for the transcription factor ARNT in the promotor of the concordat genes and not the discordant genes (Table 8). ARNT functions as a potent coactivator of estrogen receptor-dependent transcription and has also been identified as the beta subunit of a heterodimeric transcription factor, HIF-1a (Brunnberg S et al 2003).

### Significant Normal RRR Pathways and Processes are Discordant in RCC

The discordant genes were a distinct minority of the genes shared between RRR and RCC (23%). These include apparent pathogenesis-related genes and background noise due to the differences in organisms, tissue pathologies, methods and authors (see the on-line appendix). A GO analysis predicted that the discordant genes were to play a significant major role in insulin-like growth factor binding, heparin binding, the renal extracellular space and in organic cation transporter activity (p<0.05). These ontologies were distinctly different from those predicted for the concordant genes and thus we expect the concordant and discordant genes to be functionally different (Tables 5, 6, 7, FIG. 6). We have also identified a set of critical discordantly expressed genes associated with pathways or functions that may be required for RCC pathogenesis. Among these pathways and functions are the IGF pathway (observed as ontology as well), the HIF-VHL pathway, which is interconnected with the IGF pathway and processes as angiogenesis, fatty acid metabolism, glycolysis and ATP synthesis, mitochondrial, apoptosis, DNA repair and mRNA maturation. The significance of these changes is discussed below in the context of basic tumor biology.

EASE (ttp:apps1.niaid.nih.gov/David), analysis was performed on significant genes (Hosack D A et al., 2003). EASE uses a Fisher Exact test to estimate significance for functional classes of genes in a significant subset relative to the representation on the array. Gene ontology (GO) terms for biological process, cellular component, and molecular function were used (http://www.geneontology.org). The ontologies were crossed compared by using a a macro that we wrote in Excel and Michael Eisen Cluster program

### The IGF Pathway

We discovered that the discordant genes significantly share the ontology of insulin-like growth factor I (IGF-1) with the early pattern of RRR (tables 5, 6). This finding, obtained through GO analysis, is strongly supported by the literature and points to a significant regulatory role for the IGF-HIF-VHL pathways (Tables 4, 7, 9, FIG. 6). We found that IGFBP-1, -3 and -4 are down-regulated during the early pattern of RRR. In our study IGF-1R was not printed on the array, but in the with the literature was reported as down-regulated, unchanged and up-regulated in RRR, possibly influenced by the type and severity of the renal injury and the nutritional intake of the animal (Bohe J. et al 1998). Discordantly, in RCC the expressions of IGFBP-1, -3 and IGF-1R are up-regulated, a phenomenon that could in part, be attributed to the up-regulation of the HIF1a protein as a result of the loss of VHL (Table 9), (Schips L et al (2004)). Another discordantly expressed IGF-1 weakly-binding-protein was CTGF (IGFBP-8), which was up-regulated during the late pattern of RRR, but down-regulated in RCC. CTGF has the capacity to bind IGF-1 via its IGF-binding domain, albeit with relatively low affinity compared with classical IGFBPs. CTGF and IGF-1 cooperate in their upregulation of collagen type I and III expression in human renal fibroblasts. The synergy between CTGF and IGF-I might be involved in glucose-induced matrix accumulation, because both factors are induced by hyperglycemia (Lam S et al 2004).

The IGF1 signaling pathway controls cellular proliferation and apoptosis, and high §0 levels of circulating IGF-1 are associated with increased RRR and risk of several common cancers (Bohe J. et al 1998, Pollak M N et al 2004). There is a profound body of evidence to suggest that the neoplastic progression, particularly in RCC, might be associated with increased expression of IGF-1 and the receptor for IGF-1 (IGF-1R) Parker A S et al 2003, Schips L et al (2004)). The expression of IGF-1 together with its receptor, IGF-1R, provides evidence for the existence of an autocrine-paracrine loop of tumor cell stimulation in RCC and makes this type of cancer a candidate for therapeutic strategies aimed to interfere with the IGF pathway (Schips L et al (2004)). IGF-1 bioavailability is modulated by IGF binding proteins (IGPBPs) in both the circulation and the cellular microenvironment. There are opposing models regarding the regulatory role of IGFBPs in IGF-1-induced mitogenic activity. The simplest suggests that IGFBs act as competitive inhibitors which deprive receptors of their ligands (Pollak M N et al 2004). An alternative model claims that IGFBPs can enhance neoplastic behavior, while reduced IGFBPs expression can inhibit tumor growth (Pollak M N et al 2004, Renehan A G et al 2004, Dupont J et al 2003).

The HIF-VHL pathway

The majority of kidney cancers are caused by the mutation of the von Hippel-Lindau (VHL) tumor suppressor gene. The VHL protein (pVHL) is part of an E3 ubiquitin ligase complex called VEC that is composed of elongin B, elongin C, cullin 2, NEDD8, and Rbx1. VEC biomarkers a HIF transcription factor for ubiquitin-mediated destruction by oxygen-dependent prolyl hydroxylation (PHD1, 2, 3/EGLN 2, 1, 3). In the absence of wild-type pVHL—as occurs in both VHL patients and the majority of sporadic cases of clear cell renal cell carcinoma—HIF-responsive genes are inappropriately activated under normoxic conditions (Sufan R I et al 2004).

Following renal ischemia injury, we found 17 genes to be HIF-responsive in the processes of RRR (p<0.05), 7 of which proved to be discordantly expressed in RCC (p<0.05), (Table 4, 5). Interestingly, another discordant genes we identified are the PHD2/EGLN1 and PHD3/EGLN3 which are up-regulated in RCC (Jiang Y et al (2003), Boer et al (2001)), but down-regulated together with EGLN2 throughout the RRR process (Table 9, FIG. 9). Based on our probabilistic promoter analysis of the differentially expressed genes associated with RRR (data not shown), we suggest that PHD2/EGLN1 down-regulation may be attributed to thyrotrophic embryonic factor TEF/VBP, a transcription factor that regulates developmental stage-specific gene expression. TEF has been shown to be closely related to the HLF of the E2A-HLF fusion gene, formed by a (17; 19)(q22; p13) translocation (Inaba T et al 1992). This fusion product binds to its DNA recognition site not only as a homodimer but also as a heterodimer with TEF (Inukai T et al 1997). Thus, TEF could possibly play oncogenic roles in both the HIF pathway and E2A-HLF activity.

Another discordantly expressed gene belonging to the HIF pathway that was identified in our study is the WD repeat and SOCS box-containing 1 (WSB1, RIKEN 2700038M07 gene pending), which is up-regulated during the late pattern of RRR, but down-regulated in RCC. Kamura T. et al. have shown that VEC, SOCS1, and WSB1 are capable of assembling with the Cu15/Rbx1 complex. Cu15 and Cdc34 are HIF1a, E2 ubiquitin-conjugating enzymes (Kamura T et al 2001). Thus, the even though EGLN1 and 3 are up-regulated in RCC, the down-regulation of WSB1 may impair assembly with the Cu15/Rbx1 and therefore ubiquitylation by the E2 ubiquitin-conjugating enzyme Ubc5.

We also found a discordant gene, UBE2V1/CIR1, which is a variant of the ubiquitin-conjugating E2 enzyme. UBE2V1 is thought to be involved in the control of differentiation by altering cell-cycle behavior. Up-regulation of UBE2V1 expression has been found following cell immortalization in RCC and in tumor-derived human cell lines (Ma L et al 1998). We found that this enzyme is down-regulated throughout the process of RRR. Further studies are needed to explore the connection, if any, with the HIF1a, E2 ubiquitin-conjugating enzymes, Cu15 and Cdc34.

The histone deacetylase 1 (HDAC1) expression is down regulated during the late pattern of RRR and is yet to be examined in RCC. Several lines of evidence suggest that HDAC expression in up-regulated in RCC. The HIF1 complex is often over expressed in RCC because of the loss of the VHL protein and hypoxia. Under these conditions HDAC expression is expected to be up-regulated, possibly by the regulation of the HIF1 transcription complex (Kim, M S et al (2001)). Importantly, patients with renal cell carcinoma and other tumors treated with HDAC inhibitors showed some degree of clinical improvement (Sasakawa Y et al (2003), Drummond D C et al (2004)). The association of VHL protein with HDAC-1, HDAC-2, and HDAC-3 provides a molecular basis for the repression of the HIF1a transactivation domain function under nonhypoxic conditions. Interestingly, HDAC1 mRNA and protein expression are induced by hypoxia, suggesting that HDAC1 may represent a HIF-1 biomarker gene and that increased HDAC activity may contribute to the overall decreased rate of transcription in hypoxic cells (Kim M S et al. (2001), Mahon P C et al (2001)). Further, the HDAC interacts with retinoblastoma tumor-suppressor protein and this complex is a key element in the control of cell proliferation and differentiation. Together with metastasis-associated protein-2, it deacetylates p53 and modulates its effect on cell growth and apoptosis. (Luo, J et al 2000, Magnaghi-Jaulin, L et al (1998)). Interestingly, another histone deacetylase gene that we observed in our study is the Sirtuin 7 (SIRT7), which is discussed with respect to DNA repair. SIRT7 is presumably also a discordant gene and in cultured neuronal cells is reported to be up-regulated following modification of histone/protein acetylation status by several class I and II HDAC inhibitors (Kyrylenko S et al (2003)). The biological role of HDAC1 is epigenetic and complex, but the net effect of HDAC 1 over-expression is to stimulate angiogenesis and control of cell proliferation and differentiation.

A novel pathway that specifically suppresses downstream HIF-1 signaling by stress granules has recently been identified by Moeller B J et al (2004). In these granules, the up-regulation of the key stress granule scaffolding proteins, TIA1 cytotoxic granule-associated RNA binding protein (TIA1) and TIA1 cytotoxic granule-associated RNA binding protein-like 1 (TIAL1/TIAR), results in hypoxia-mediated translational decrease. In contrast, in the presence of free radical species (ROS) the stress granules depolymerizes, the downstream HIF-1 signaling is enhanced, leading to increased translation of HIF-1-regulated transcripts as VEGF. ROS is formed following radiation therapy, RCC pathogenesis and RRR and thus HIF translational silencing is expected to be impaired. During early RRR, TIAL1 is up-regulated and presumably involved in gene transcriptional silencing. During late RRR TIAL1 expression reverts to normal levels, thus mediating the translation of HIF-1-regulated transcripts.

We also found that the gene Nulp1 (KIAA1049), a basic helix-loop-helix protein, is discordantly expressed. Nulp1 is down-regulated during early RRR, but is up-regulated both in RCC and during early embryonic organogenesis (Table 9) (Olsson M et al 2002). Interestingly, Nulp1 and ARNT (HIF-1b) proteins can bind to and activate transcription from promoters driven by the CACGTG E-Box element. This activation is potentially repressed by the HIF regulated inhibitor of D binding 2 (ID2), which is concordantly up-regulated in RCC and at the late pattern of RRR (Table 9). (Scobey M J 2004, Lofstedt T et al 2004).

HIF1 activates the transcription of genes that are involved in crucial aspects of cancer biology, including angiogenesis, cell survival, glucose metabolism and invasion (Semcaca G L 2003). Both intratumoral hypoxia and the genetic alterations induced by the genetic discordantly expressed genes discussed above can lead to HIF1a overexpression, which has been associated with increased patient mortality in several cancer types, including RCC.

### Angiogenesis

Tumor angiogenesis differs significantly from normal angiogenic processes several important respects, including aberrant vascular structure, altered endothelial-cell-pericyte interactions, abnormal blood flow, increased permeability, and delayed maturation. The onset of angiogenesis, or the “angiogenic switch,” is a discrete step that can occur at any stage of tumor progression, depending upon the tumor type and characteristics of its microenvironment (Bergers G, Benjamin L E. (2003)). In RCC, the angiogenic factor VEGFA and its receptor KDR/VEGFR2 are up-regulated, but both genes are down-regulated at the early pattern of RRR and VEGF throughout the late pattern as well (Table 7). These findings are supported by the reports that in RRR—unlike in other organs—VEGF is primarily up-regulated at the post-transcriptional level (Vannay A et al (2004), Kanellis J et al (2000), Lemos F B et al (2003)). On the other hand, the endothelial VEGFR2, but not VEGFR1, was reported earlier to be up-regulated in rats RRR (Kanellis J et al (2000)). Hypoxia-dependent VEGF up-regulation in carcinoma is attributed to the up-regulation in HIF1a protein consequent to the loss of VHL, and VEGF down-regulation in wound healing could result from a synergistic interaction among multiple regulatory transcription factors and/or inhibitors capable of overcoming HIF1a induction (FIG. 7, Table 9). These observations indicate that the discordant expression of the pro-angiogenic genes VEGFA and KDR are very likely to play a central role as an onco-angiogenic switch during RCC pathogenesis.

### Fatty Acid Metabolism

Fatty acid metabolism plays a major role in cancer. Our study found that two fatty acid metabolic enzymes, Acyl-Coenzyme A oxidase 1 (ACOX1/1.3.3.6) and Carnitine PalmitoylTransferase 1A (liver) (CPT1A/2.3.1.21) are up-regulated in RCC, but down-regulated during the late pattern or continually during RRR (respectively). The over-expression of both enzymes may increase the levels of intracellular H2O2 and therefore may act analogously to other carcinogenic ROS (Okamoto M, et al 1997).

### Glycolysis and ATP Synthesis

Fast-growing tumors depend largely upon glycolysis for ATP generation. In hypoxic solid tumors, ATP is replenished through glucose oxidation by the anaerobic glycolytic pathway, even though this pathway is far less effective in ATP production than is aerobic glucose oxidation (Frydman, B. et al., 2004). Our comparison between RCC and RRR indicates major differences in the expression of certain glycolytic genes:

The enzymes hexokinase 1 (HK1) but down-regulated during early RRR. HK1 phosphorylate glucose produces glucose-6-phoshate, thus in RCC committing glucose to the glycolytic pathway (Tables 7, 9). Another enzyme in the glycolytic pathway, the phosphofructokinase Liver (PFKL) proved to be down-regulated in the early pattern of RRR and its expression in RCC is yet to be determined. PFK catalyzes a key step in glycolysis, namely the conversion of D-fructose 6-phosphate to D-fructose 1,6-bisphosphate. In kidney, HK1 and PFKL are expressed in the PRT and are regulated by HIF1a and possibly by p53 (Table 9). In many tumors, HK1 and PFKL are unleashed to supply the cell with ATP (Eigenbrodt, E. et al., 1992, Nakamura, K., 1988, Semenza, G. L. et al., 1994).

To stimulate continued glycolytic flux and prevent toxic effects, lactate must be eliminated from the cell. This process is mediated by the monocarboxylate transporter (MCT). In RCC, SLC16A7/MCT2 is up-regulated, while in normal RRR it is down regulated, an observation that further supports the notion that tumor cell is programmed to maintain continued glycolytic flux and prevent toxic effects (Lin, R et al 1998; Halestrap A P and Price N T 1999).

We also found three genes associated with purine metabolism are discordantly expressed in RSS and during RRR: the fragile histidine triad (FHIT), the ribonucleotide reductase M1 polypeptide (RRM1) and ectonucleotide pyrophosphatase/phosphodiesterase 2 (autotaxin), (ENPP2). FHIT is inactivated in many of the common human malignant diseases and it is localized close to the renal tumor suppressor gene, VHL. FHIT is either down-regulated or deleted in RCC but highly expressed in all normal epithelial tissues and is up-regulated during RRR (Tables 7, 9).

RRM1 is up-regulated in RCC in down-regulated in the early pattern of RRR (Tables 7, 9). RRM1, also, catalyzes the activity of thioredoxin (TXN), which expression is up-regulated in RRR. The literature describing the TXN expression pattern in RCC is contradictory: some reports have indicated that the gene is down-regulated, while other studies have offered evidence suggesting that it is up-regulated (Tables 7, 9). We have found that two members of the thioredoxin family possess distinctly different expression patterns during different patterns of RRR: thioredoxin-like (TXNL) is up-regulated during the early pattern of RRR, while thioredoxin 2 (TXN2) is down-regulated during the late pattern of RRR. TXN2 plays an important role in protecting mitochondria from oxidant-induced apoptosis and its down-regulation therefore serves to switch on the apoptosis process (Chen, Y. et al., 2002). Nonetheless, we have yet to clarify the role of the differential TXN expression in RCC

Ectonucleotide Pyrophosphatase/Phosphodiesterase 2 (autotaxin), (ENPP2) is down-regulated continuously throughout the process of RRR, but elevated in RCC and other tumors (Tables' 7, 9). ENPP2 is an extracellular enzyme and an autocrine motility factor that stimulates pertussis-toxin-sensitive chemotaxis in human melanoma cells at picomolar to nanomolar concentrations. ENPP2 processes 5′-Nucleotide phosphodiesterase/ATP pyrophosphatase and ATPase activities that potently induce tumor cell motility, and enhance experimentally induced metastasis and angiogenesis (Clair, T., et al., 2003).

During early RRR, phosphofructokinase-Liver (PFKL) is down-regulated and returns to normal levels during the late pattern of RRR (Tables 7, 9). Presumably, the rate of glycolysis is normally greatly in excess (greater than 400-fold) of that required for biosynthetic processes. Therefore, PFKL is first down-regulated, and then restored back to the normal level or to the level that is needed to meet any new ATP demand (Newsholme E A and Board M 1991). Further studies are needed to evaluate the PFKL expression in RCC.

A localized increase in ADP, which stimulates glycolysis and ATP production is generated by the SLC1A1/EAAC1 turnover (Welbourne and Matthews 1999). During the late pattern of RRR SLC1A1 expression is up-regulated, but in RCC, it is down-regulated. A decrease in the expression of SCLCA1 may slow the glycolysis and presumably results in further ATP deficit.

When O₂ is limiting, cells switch from oxidative phosphorylation to glycolysis as the primary generator of ATP (Pasteur effect). In hypoxic tumors as RCC, the constitutive stabilization of HIF in Vhl−/− cells together with the discordant expression of genes in the HIF-IGF pathway, further increases the hypoxic response of these cells. Therefore, in RCC the expression of key glycolytic genes is altered to meet the cell ATP needs. The discordant expression of these genes in RCC Vs. RRR may represent a normal glycolysis that gone awry.

### The Mitochondria

Mitochondrial defects have been associated with neurological disorders, as well as cancers. Two ubiquitously expressed mitochondrial enzymes succinate dehydrogenase (SDH) and fumarate hydratase (FH, fumarase) catalyze sequential steps in the TCA cycle. SDH is a component of complex II of the respiratory electron-transport chain. Germline heterozygous mutations in the autosomally encoded mitochondrial enzyme subunits SDHD, SDHC and SDHB cause the inherited syndromes phaeochromocytoma and paraganglioma. In RCC the expression of the SDHB gene is down regulated, which is in concordance with the data we have derived from our RRR set indicating that SDHA and SDHB are down-regulated during the early pattern of RRR (Table 9). Partial or complete loss of SDH or FH activity leads to energy depletion, free-radical formation and is sensed by the mitochondria as hypoxia. This leads to stabilization of HIF-1, its translocation to the nucleus and activation of its biomarker genes and possibly loss of mitochondrial-mediated energy-dependent apoptosis (Eng C, et al., 2003). Once the mitochondrial outer membrane is breached or undergoes a change in composition because of the ROS, an energy-independent apoptotic cascade occurs that involves release of cytochrome c and procaspases (Eng C, et al., 2003). The gene encoding to the cytochrome c oxidase subunit VIc (COX6C), is also differentially expressed during the early pattern of RRR, where it is down-regulated, as apposed to RCC, where it is up-regulated. COX6C is a subunite of the cytochrome c oxidase (COX), the terminal enzyme of the mitochondrial respiratory chain that catalyzes the electron transfer from reduced cytochrome c to oxygen. Thus a discordant over-expression in RCC may impact this catalysis.

These discordant genes collectively constitute the first detailed global molecular comparison of the pathways and cellular process generating the energy balance during RRR and RCC. These findings support the Warburg hypothesis suggesting that the cause of cancer is primarily a defect in energy metabolism (Warburg, O 1956). Through numerous studies it has become apparent that tumor cells rely to a greater extent on glycolytic pathways than do normal cells even in the presence of abundant oxygen. While it is clear that the metabolism of cancer cells is different from that of normal cells, our work identified the candidate genes distinguishing the metabolism of RRR from RCC.

It is conceivable that partial decreases or chronic, low-level reductions in energy production, which are insufficient to cause overt symptoms but could contribute to inefficient energy-dependent apoptosis (van Loo, G. et al 2002; Ravagnan, L. et al 2002, Eng C, et al., 2003). Thus the subsequent impact of a discordant gene in the energy balance could lead to complete loss of energy-dependent apoptosis and therefore to cancer promotion

### DNA Repair

DNA repair mechanisms can be induced under a variety of physiological and pathological conditions. We identified a number of discordantly expressed genes-prominent among which are SMC1L1, TOP3B, and SIRT7-suggesting that certain alterations in DNA repair mechanisms play an important role in RCC pathogenesis discordant genes also exemplified possible alterations in the DNA repair:

The structural maintenance of chromosomes 1-like 1 (yeast) (SMC1L1), is up-regulated during the early pattern of RRR, but down-regulated in RCC (Tables 7, 9). As part of the cohesin complex, the protein encoded by SMC1L1 is essential for sister chromatid cohesion in yeast cells undergoing mitosis. In addition, the protein has a potential role in DNA repair (Sumara, I. et al 2000).

Another discordantly expressed gene involved in DNA repair was the topoisomerase (DNA) III beta (TOP3B), that is down-regulated during the early pattern of RRR, but up-regulated in RCC (Tables 7, 9). This gene encodes a DNA topoisomerase, an enzyme that controls and alters the topologic state of DNA during transcription. The TOP3B enzyme catalyzes the transient breaking and rejoining of a single strand of DNA, allowing the strands to pass through one another, by relaxing the supercoils and altering the topology of DNA. The enzyme interacts with DNA helicase SGS1 and plays a role in DNA recombination, cellular aging, and the maintenance of genome stability (Li W and Wang J C 1998).

Sirtuin 7 (SIRT7) may represent another discordantly expressed DNA repair gene involved in RCC pathogenesis, but it needs to be studied further before such a role can be confirmed. We observed that SIRT7 is down-regulated at the early pattern of RRR (Table 9). We have gathered evidence that the gene is up-regulated in carcinoma of the thyroid but have yet to acquire data confirming that it is similarly unregulated in RCC. Sirt7 is a member of the sirtuin family of proteins, which are homologs of the yeast Sir2 proteins (Sir1-7). The functions of human sirtuins have not yet been determined; however, yeast sirtuin proteins are associated with calorie intake, regulation of metabolic rates, chromatin regulation, and DNA recombination. It has been suggested that SIRT 1 promotes the long-term survival of irreplaceable cells (North B J et al 2004, North B J et al 2004, Cohen H Y et al 2004). Thus discordant expression of genes involved in DNA repair could result in accumulation of mutations and genome instability.

mRNA Maturation

One of the key events that takes place in the nucleus during mRNA maturation is the polyadenylation of the 3-prime end of eukaryotic mRNA. We observed that the poly(A) polymerase (PAPOLA/PAP) is continuously down-regulated throughout the process of RRR, but up-regulated in RCC (Table 9). This discordant gene is of particular interest as high levels of PAPOLA activity are associated with rapidly proliferating cells, the enzyme exerts anti-apoptotic effects and it has been identified as an unfavorable prognostic indicator in leukemia and renal cancer (Stetler D A et el 1981, Balatsos N A et al 2000). Thus, we suggest that the discordant genes are also involved in the deregulation of mRNA in the tumor cells.

### The Extracellular Space

Our set of discordant genes also significantly shared the ontology of the ECM. We found five of the six genes in this ontology to be up-regulated, with a pattern of expression similar/identical to that of trends 5 and 6, both of which are up-regulated at two weeks (Tables 5, 6, 7, 9, FIG. 6). Normal cells remain confined to their home territory because they are held in check through an interchange of signals with neighboring cells and the surrounding ECM. In contrast, successful malignant tumor cells have been hypothesized as being resistant to such regulatory signals as a result of appropriating, misinterpreting, or disregarding the signals during the invasion of local host-cell populations (Liotta L A and Kohn E C. (2001)).

The ECM genes we found to be up-regulated during the late pattern of RRR, but down-regulated in RCC—APOE, CTGF/IGFBP8, DCN, GPC3, PLAT, and THBS1—all appear to be play distinct roles in the malignant cell's complex process of becoming resistant to regulatory signals originating from surrounding cells and/or the ECM.

Down-regulation of APOE appears to slow microtubule polymerization in vitro (Scott B L et a 1998), and thus may affect the growth and behavior of malignant cells as in RCC tumor (Lenburg M E et al (2003), Boer J M et al (2001), Galban S et al (2003), Vogel T et al 1994, Ishigami M et al 1998). Down-regulation of CTGF may inhibit CTGF induced mesangial cell migration in RCC (Crean J K et al 2004)

DCN, the third discordant ECM gene, encodes the pericellular matrix proteoglycan, decorin, a protein component of connective tissue that binds to type I collagen fibrils. It plays a role in matrix assembly and is capable of suppressing the growth of various tumor cell lines (Moscatello, D K et al 1998).

Mutations in the fourth discordantly down-regulated gene, GPC3, may have a possible role of in Wilms tumor development and in an overgrowth disorder, Simpson-Golabi-Behmel syndrome, that may be independent of IGF signaling (White G R et al 2002; Lindsay S et al 1997, Chiao E et al 2002).

The fifth gene, PLAT, is a serine protease that activates the proenzyme plasminogen to yield plasmin, which has fibrinolytic activity. Increased plasmin activity causes hyperfibrinolysis, which manifests as excessive bleeding; decreased activity leads to hypofibrinolysis, which can result in thrombosis or embolism (Jorgensen et al. (1982)).

The final gene of this group, THBS1, encodes an adhesive glycoprotein that mediates cell-to-cell and cell-to-matrix interactions. The protein has been shown to play roles in platelet aggregation, angiogenesis, and tumorigenesis. Moreover, IGF2 over-expression a common genetic alteration of adrenocortical carcinomas, has been significantly correlated with both higher VEGFA and lower THBS1 concentrations (De Fraipont et al. (2000)).

### The Organic Cation Transporter

The organic cation transporter, solute carrier family 22 (SLC22A1), is critical for the elimination of many endogenous small organic cations, as well as a wide range of drugs and environmental toxins, in kidney and other tissues. SLC22A1 is up-regulated in RCC, but down-regulated in RRR (FIG. 9). It may play a role in eliminating toxins—and possibly anticancer—drugs from carcinoma cells but lack an analogous function in normally regenerating kidney cells (Shu et al. (2003)).

### Specific Pathways are Activated During RRR and in RCC

In both RCC and healing wounds, hypoxia alters overall cellular behavior as a consequence of, or in addition to, activating specific genetic pathways, such as HIF-VHL, MYC, p53, IGF and NF-kB (Elson D. A. et al., 2000, Maxwell P H. 2004, Schips L et al 2004, Hammerman M R 1999, Yamaguchi S et al 2003, Koshiji M et al 2004, Schmid T et al 2004, Qi H and Ohh M2004, Cao C C et al 2004) (Table 4, FIGS. 5, 6). Our observations have shown that several concordantly expressed genes are significantly regulated by hypoxia and the pathways of VHL Myc, p53 and NF-kB, but not by the interconnected pathways of IGF and HIF (P<0.05). These findings indicate that the VHL gene plays a significant role not only in HIF-dependent pathways, but also in some pathways independent of HIF (Wykoff C C et al 2004). Added to this observations, our probabilistic functional genomics comparison of the concordantly and discordantly expressed genes between RRR and RCC (Table 8) suggests a distinct enrichment (loading of 4.169418) of ARNT homdimer element (5′-CACGTG-3′) in the predicted promotor region regulating the expression of the concordant genes (30 genes) and less in the discordant genes (6 genes). 7 genes, 6 of them concordantly expressed were reported in the literature to be regulated by Myc (Table 8). The c-Myc/Max hetrocomplex and the ARNT/ARNT hetrocomplex interact to the same DNA recognition but with different affinity (Swanson H I and Yang J H 1999). ARNT proved to be capable of homodimerizing as well participating in multiple partnerships resulting in a diversity of DNA recognition sites. Partners of ARNT include AHR, SIM1, SIM2, HIF-1a, HIF-2a and CHF1, regulators of xenobiotic-metabolizing enzymes (as cytochrome P450), neurogenesis, the cellular response to hypoxia and cardiovascular angiogenesis, respectively. In this manner, ARNT serves as a central player in regulating these divergent signaling pathways (Swanson H I (2002)).

In comparison to the concordantly expressed genes, the discordantly expressed genes are also significantly regulated by hypoxia and the pathways of Myc and p53, but not by the NF-kB. Moreover, while ARNT homodimer is distinctly enriched to be a regulator of the concordantly expressed genes, the discordantly expressed genes are distinctly regulated by the ARNT heterodimer with HIF-1a pathway regulated by IGF and VHL pathways (Tables 4, 7 and 8). Further, it is implied from our promotor analysis that EGLN1, which is involved in HIF-1a and HIF-2b ubiqutination, is subject to regulation by the ARNT homodimer.

To better comprehend the complexity of the intricate bioregulaory network we have been studying, we have formulated a Molecular Interaction Map that integrates the pathways we have extrapolated from ontology studies, probabilistic functional genomics analysis, and our survey of the literature (FIG. 7). This core map (Riss, J., Kohn, K. W., et al., 2004—review in preparation) demonstrates that normal and oncogenic regeneration are regulated by the same pathways and that the failure of a critical angiogenic master switch can provide the transformed cell with a selective growth advantage. Among these pathways are the VHL-HIF1a, IGF, Myc, P53, NF-kB and others that provide the biosystem with functional redundancy, which is enabled by cellular heterogeneity, and feedback-control systems that are used to facilitate survival in hazardous environments, such as those resulting from some anticancer drugs or hypoxia) (Kitano, H., 2004).

### Perspective and Future Work

To our knowledge, we have described for the first time, a coherent set of molecular similarities and differences between normal RRR and RCC that, taken together, suggest the existence of a novel molecular mechanism as the aberration of a normal phenotype rather than as a lapse into chaos. The molecular aberration is in gene mutations (i.e. VHL), transcription control (i.e. the discordantly expressed PHDs genes in the VHL-HIF-1a-ARNT pathway), in the autocrine-paracrine loop regulation of tumor cell stimulation (i.e. the discordantly expressed IGFBP-1, -3, genes) and epigenticaly (possibly discordant expression of the Sirt-7 and HDAC genes). The molecular aberrations lead to phenotypic aberrations in vital denominators of RRR and RCC, as in DNA repair, mRNA maturation, glycolysis and ATP synthesis, fatty acid metabolism, mitochondria, extracellular space and organic cation transporter. Collectively the phenotypic aberrations offer growth advantage needed for the RCC.

Such an insight proves of great utility in the development of therapeutic strategies to treat cancer. For example, it is possible that genes expressed concordantly in RRR and RCC may permit the tumor to respond to certain physiological signals that are known inhibit tissue regeneration. Therapeutic agents similar to such signaling molecules (i.e., initiation of DNA replication) could be developed and would perhaps have effects that would be more predictable and consistent than those of conventional agents. A few such agents are now under investigation (Riss J et al 2005, manuscript in preparation).

Another highly tempting biomarkers for intervention include the discordantly expressed genes that distinguish RRR from RCC. These genes could become the basis for biomarkering the drugs to the tumor cells, but not the normal regenerating cells (Riss J et al 2005, manuscript in preparation). Another highly tempting biomarkers for intervention include the discordant bioenergic balance in the tumor cell (Kribben A et al 2003; Agteresch H J et al 1999). Further, the discordantly expressed genes could also become the basis for the development of improved RCC biomarkers for early detection and diagnosis (Riss J et al 2005, manuscript in preparation).

Finally, the findings presented here may have implications for the improved treatment of other diseases or disorders as ARF, kidney transplantation and possibly other types of malignant neoplasms that have been described in the literature as associated with trauma, chronic wounding, and inflammation.

### Implementation of Comparative Biology in the Current Study

RRR vs. RCC

RRR though common in human (i.e. kidney transplantation) 0 is extremely difficult for obtaining time course viable samples. Therefore, the changes in RRR gene expression are evident from rodent models and have been less systematically studied in human. Alternatively, to the best of our knowledge no mouse model is available for sporadic RCC ( ). This hurdle can be overcome by a careful comparative biology analysis of the uniformity and diversity in the gene expression of RRR and RCC of mouse and human (respectively).

In the current study we integrated data from different organisms, tissue pathologies, methods and authors. The interspecies comparison of gene expression of mouse RRR with human RCC was feasible by using the normal tissue in each original publication as a reference point. The significance of the differentially expressed genes was as offered by the authors.

The feasibility of the comparison was supported by the findings that both the RCC and the RRR process are predominantly found in the proximal tubules (FIG. 2), (Price, P. M. et al., 2003 Add ref for RCC). Therefore, and based on the literature, many genes in the current data set were also cataloged for their tissue topological expression (Table 9). In terms of cell replication, both tumors and regenerating tissue contain four populations of cells: (1) cycling cells, (2) cells that can be recruited into cycling, (3) cells unable to divide because they are partially differentiated and (4) dying or apoptotic cells (Stell, 1967, 1977).

### Noise Reduction

To reduce the noise in the results of the interspecies extrapolation, the differential expression was catalogued and compared only qualitatively (not quantitatively), as expressed up or down from normal tissue (FIG. 9). Therefore the interspecies extrapolation of differentially expressed genes in mouse RRR and human RCC identified a core signature, which collectively (concordant and discordant genes) is conserved through both evolution and renal pathologies.

The concordance and discordance qualitative expression is a result of the inherent similarities and differences between mouse, human, RRR and RCC. The concordance between mouse RRR and human RCC at 77% supports comparability of data across species and pathologies, while the discordance at 23% indicate the difference between mouse RRR and human RCC. Both groups of genes clustered into distinct ontologies pathways and were mostly in agreement with the literature (p<0.05). The significance for concordant and discordant genes is high (p-value 2.2e-16, binomial test).

Finally, we validated our RRR data set by comparing it with the literature, QPCR and immunohistochemistry (Table 9, FIGS. 2, 9). The comparison with the literature clearly demonstrated the power of using the normal tissue as a reference point. A comparison of the RRR literature with the current RRR dataset identified 91 genes that appeared on both lists. 89% of these genes were in full agreement with the literature, despite the difference in organisms (human, rat, mouse) and methods (Table 9).

Therefore, qualitative data integration is plausible if the normal tissue is used as a reference point and is subject to filtering for qualitative gene expression that is conserved in evolution and further widely correlated with the literature and or experiments.

### Comparison of Literature Knowledge and Our Experimental Data

To incorporate into our analysis the literature knowledge on RRR and RCC, we catalogued and referred these data. First we gathered the known genes to participate in the pathways of the genes: von Hippel-Lindau (VHL), HIF, insulin-like growth factor (IGF), tumor protein p53 (TP53), nuclear factor of kappa light polypeptide gene enhancer in B-cells (NF-kB), the v-myc myelocytomatosis viral oncogene homolog (MYC) and the genes in the purine metabolism pathway. Then, we catalogued the genes that were reported to be differentially expressed in hypoxia versus normoxia, as well as the genes presumably involved in cell senescence. These are two of the major physiologic conditions in cancer and tissue regeneration and are of much interest for further studies. Next, we cataloged the known genes to be differentially expressed in pathologies as RCC, RRR, and metastasis and those suggested to be involved in pathways on oncogenes and/or tumor suppressors. Last, we referenced the literature knowledge on genes expression and renal histology. These databases were compared with the current RRR dataset and a comprehensive cross-comparison is presented in table 9.

### Validation of the Microarray Dataset

A global knowledge step toward constructing a RRR systems biology network model is to build a comprehensive RRR expression database. Therefore we reviewed the evidence reported in the literature on differentially expressed genes in RRR and the relevant pathways and cross-compared them with the current study (table 9). Of the 1325 RRR differentially expressed genes in the current study, the expression of 91 genes was previously compared with normal kidney. The qualitative expression of 89% of the 91 genes was in full agreement and only 11% was in qualitative conflict that included the genes: NID, NRP1, ZFP36L1, TNC, MAPK1, HSPD1, HK1, NEDD4, CASP1 and UK114. These results were despite the difference in organisms (human, rat, mouse) and methods (Table 9). We further validated the data by RT-QPCR of PHD2 (EGLN1) that was at least 5-fold down-regulated in early and late regenerating kidney in comparison to resting/normal kidney. Similar expression patterns were repeated with two other related prolyl hydroxylases, PHD1 and PHD3 that were at least two-fold down-regulated (FIG. 9).

Lastly, The MiB-1 high expression at 2 days was in full agreement with the array results (Table 9).

TABLE 1
The RRR gene expression distribution: 14% of the genes
were differentially expressed
The GEM2 mouse cDNA array was printed with 9646 spots genes.
1350 spots, corresponding to 1325 genes differentially expressed
between normal-ischemic kidneys, and regenerating kidneys. The
differential gene expression is presented here as up or down in
regenerating Vs normal-ischemic kidney.

| | | % of genes | | |
|---|---|---|---|---|
| | Total | (9646) | Up | Down |
| GEM2: printed spots | 9646 | 100% | N.A. | N.A. |
| Uniquely changed | 1325 | 14% | 802 | 523 |
| Early (A) | 629 | 7% | 336 | 293 |
| Late (B) | 373 | 4% | 227 | 96 |
| Early & late (*) | 323 | 3% | 189 | 134 |

Table 2: an Ontology Analysis in Timely Dependent Fashion: Distinct and Common Ontologies

The differentially expressed genes were clustered according to their pattern of expression as early, late or continually RRR. Functional ontology was analysis performed (Fisher Exact p<0.05). The average expression of each ontology is presented in a green to red scale; green down-regulated, red up-regulated. See the supplemented table 10 for a further detailed table

TABLE 3
Association of differentially expressed genes during RRR and with known
pathways of RRR
Based on the literature, the genes in known pathways of RRR were catalogued into
datasets (category). The genes in each dataset that were printed on the GEM2 array are given
in column A and the differentially expressed genes are given in column B. Also given for
each category the relative part from the whole differently expressed gene (1325) and from the
genes belonging to that category and are printed on the array. The p value is p < 0.05.

| | | Category size | No. of genes that | | % of genes in | |
|---|---|---|---|---|---|---|
| | | (No. of genes) | are changed in | % of all changed | the category | |
| No. | Category | [A] | renal regeneration [B] | genes (1325 genes) | [B/A] | p value |
| 1 | Total No. of | 5796 | 1325 | 100 | 23 | N.A. |
| | gen [symbol] | | | | | |
| 2 | VHL pathway | 282 | 104 | 8 | 37 | <0.0001 |
| 3 | Hypoxin | 251 | 95 | 7 | 38 | <0.0001 |
| | pathwa [symbol] | | | | | |
| 4 | HRB target (HIF) | 39 | 17 | 1 | 44 | 0.0037 |
| 5 | IGF pathway | 139 | 37 | 3 | 27 | 0.3341 |
| 6 | Myo pathway | 368 | 136 | 10 | 37 | <0.0001 |
| 7 | p53 pathway | 1259 | 262 | 20 | 21 | 0.0548 |
| 8 | NF-kB pathway | 200 | 52 | 4 | 26 | 0.322 |

[symbol]

indicates data missing or illegible when filed

### Table 4: the Differentially Expressed Genes in RRR and RCC are Regulated Similarly

984 genes, printed on the array, were previously described to be differentially expressed in RCC from normal kidney. These genes were qualitatively crossed compared with the current microarray study identifying 1325 RRR differentially expressed genes from normal kidney. 361 genes are expressed in both RRR and RCC (A), 278 concordantly expressed genes (B), and 83 discordantly expressed genes (C).

Based on the literature, the genes in known pathways of RRR and RCC were catalogued into datasets (category). The number of genes in each dataset that were printed on the GEM2 array are given in column A; the number of differentially expressed genes are given in column B and in column C are given the number of the genes changed in both RRR and RCC. Also given for each category the relative part from the whole differently expressed gene in both RRR and RCC (361 genes), RRR (1325 genes) and from the genes belonging to that category and are printed on the array. The p-value for observing the concordance(77% reg/RCC) and the discordance (23% reg/rcc) is p-value <2.2e-16. (see also FIG. 5).

TABLE 4

| | | | | | | | | |
|---|---|---|---|---|---|---|---|---|
| | | | | | | In a category: the | | |
| | | | | No. of | | % of renal | % of all the | |
| | | Category | | genes that | | regeneration | category that | |
| | | size | No. of genes that | are changed on | | genes that | is changed | |
| | | (No. of | are changed in | both renal | % of all the 361 genes | are changed on both | on both renal | |
| | | genes) | renal | regeneration | changed on both renal | renal regeneration | regeneration | |
| No. | Category name | [A] | regeneration [B] | and RCC [C] | regeneration and RCC | and RCC [C/B] | and RCC [C/A] | p value |

A. All genes changed in both renal regeneration and RCC:

| | | | | | | | | |
|---|---|---|---|---|---|---|---|---|
| 1 | RCC | 984 | 361 | 361 | 100 | 100 | 37 | <0.00001 |
| 2 | VHL pathway | 282 | 104 | 75 | 21 | 72 | 27 | <0.00001 |
| 3 | Hypoxia pathwa | 251 | 95 | 51 | 14 | 54 | 20 | <0.00001 |
| 4 | HRE target (HIF | 39 | 17 | 11 | 3 | 65 | 28 | <0.0001 |
| 5 | IGF pathway | 139 | 37 | 17 | 5 | 46 | 12 | 0.0053 |
| 6 | Myc pathway | 368 | 136 | 65 | 18 | 48 | 18 | <0.00001 |
| 7 | p53 pathway | 1259 | 262 | 112 | 31 | 43 | 9 | <0.0001 |
| 8 | NF-kB pathway | 200 | 52 | 24 | 7 | 46 | 12 | 0.001 |

B. Genes changed concordantly between renal regeneration and RCC:

| | | | | | | | | |
|---|---|---|---|---|---|---|---|---|
| 1 | RCC | 984 | 361 | 278 | 77 | 77 | 28 | <0.00001A |
| 2 | VHL pathway | 282 | 104 | 59 | 16 | 57 | 21 | <0.00001 |
| 3 | Hypoxia pathwa | 251 | 95 | 35 | 10 | 37 | 14 | <0.0001 |
| 4 | HRE target (HIF | 39 | 17 | 4 | 1 | 24 | 10 | 0.2205 |
| 5 | IGF pathway | 139 | 37 | 9 | 3 | 24 | 7 | 0.4614 |
| 6 | Myc pathway | 368 | 136 | 55 | 15 | 40 | 15 | <0.00001 |
| 7 | p53 pathway | 1259 | 262 | 80 | 22 | 31 | 6 | 0.0043 |
| 8 | NF-kB pathway | 200 | 52 | 19 | 5 | 37 | 10 | 0.0027 |

C. Genes changed disconcordantly between renal regeneration and RCC:

| | | | | | | | | |
|---|---|---|---|---|---|---|---|---|
| 1 | RCC | 984 | 361 | 83 | 23 | 23 | 8 | <0.00001A |
| 2 | VHL pathway | 282 | 104 | 16 | 5 | 15 | 6 | <0.0001 |
| 3 | Hypoxia pathwa | 251 | 95 | 16 | 4 | 17 | 6 | <0.0001 |
| 4 | HRE target (HIF | 39 | 17 | 7 | 2 | 41 | 18 | <0.0001 |
| 5 | IGF pathway | 139 | 37 | 8 | 2 | 22 | 6 | <0.0001 |
| 6 | Myc pathway | 368 | 136 | 10 | 3 | 7 | 3 | 0.0551 |
| 7 | p53 pathway | 1259 | 262 | 32 | 9 | 12 | 3 | 0.0003 |
| 8 | NF-kB pathway | 200 | 52 | 5 | 2 | 10 | 3 | 0.3217 |

### Table 5: the Differently Expressed Genes in Both RRR and RCC Exhibited Distinct Ontologies for the Concordance Vs Discordance Genes

The differentially expressed genes in both RRR and RCC were clustered according to their concordance Vs discordant change. Functional ontology was analysis performed (Fisher Exact p<0.05). The average expression of each ontology is presented in a green to red scale; green down-regulated, red up-regulated. The number of genes up-/down- regulated in both RRR and RCC is also given and the direction is as in RRR relative to the normal kidney. In terms of Sutton's renal RRR model (Sutton T A et al 2002—FIG. 1) the ontologies are related as extension (E), maintenance (M) and repair (R). See the Table 11 for detailed information.

TABLE 5

| | | | | |
|---|---|---|---|---|
| | | | | Genes |
| | | | Category | Expressed |
| | | No of Genes | Average | in RRR |
| Go System | Category | UP/DOWN | Expression | Phases |

Concordance:

| | | | | |
|---|---|---|---|---|
| Molecular Function | immunoglobulin binding | 3; 0 | 1.103 | E, M, R |
| | selenium binding | 1; 3 | −0.388 | E, M, R |
| | extracellular matrix structural constituent | 5; 0 | 0.886 | E, M, R |
| | conferring tensile strength activ [symbol] | | | |
| | structural constituent of ribosome | 23; 0 | 0.737 | E, M, R |
| | RNA binding | 27; 1 | 0.563 | E, M, R |
| | cell adhesion molecule activity | 11; 2 | 0.458 | E, M, R |
| Cellular Component | cytosolic ribosome (sensu Eukarya) | 11; 0 | 0.730 | E, M, R |
| | proteasome core complex (sensu Eukarya) | 4; 0 | 0.563 | E, M, R |
| | collagen | 5; 0 | 0.886 | E, M, R |
| | small ribosomal subunit | 5; 0 | 0.698 | E, M, R |
| | microfibril | 7; 0 | 1.029 | E, M, R |
| Biological Process | phenylalanine metabolism | 0; 3 | −1.203 | E, M, R |
| | phenylalanine catabolism | 0; 3 | −1.203 | E, M, R |
| | tyrosine metabolism | 0; 3 | −1.033 | E, M, R |
| | DNA replication initiation | 4; 0 | 0.688 | E, early M |
| | regulation of translation | 4; 2 | 0.135 | E, M, R |
| | ribosome biogenesis | 10; 0 | 0.750 | E, M, R |
| | posttranslational membrane targeting | 5; 2 | 0.491 | E, M, R |
| | cell ion homeostasis | 1; 4 | −0.506 | E, M, R |
| | ER organization and biogenesis | 6; 2 | 0.483 | E, M, R |
| | macromolecule biosynthesis | 26; 2 | 0.608 | E, M, R |
| | cytoplasm organization and biogenesis | 25; 4 | 0.656 | E, M, R |
| | death | 13; 2 | 0.523 | E, M, R |
| | cell adhesion | 18; 2 | 0.609 | E, M, R |
| | immune response | 18; 0 | 0.994 | E, M, R |
| | cell growth and/or maintenance | 74; 25 | 0.309 | E, M, R |
| | protein metabolism | 57; 8 | 0.542 | E, M, R |

Discordance:

| | | | | |
|---|---|---|---|---|
| Molecular Function | insulin-like growth factor binding | 2; 2 | 0.088 | E, M, R |
| | organic cation transporter activity | 1; 2 | −0.267 | E, M, R |
| | heparin binding | 3; 2 | 0.102 | E, M, R |
| Cellular Component | extracellular space | 12; 12 | 0.084 | E, M, R |
| Biological Process | one-carbon compound metabolism | 0; 3 | −0.517 | E, M, R |
| | angiogenesis | 3; 2 | 0.390 | E, M, R |
| | regulation of cell growth | 2; 2 | 0.088 | E, M, R |
| | actin cytoskeleton organization and biogenesis | 2; 1 | 0.177 | E, M, R |
| | actin filament-based process | 2; 1 | 0.177 | E, M, R |
| | enzyme linked receptor protein signaling pathway | 3; 2 | 0.226 | E, M, R |
| | organelle organization and biogenesis | 3; 6 | −0.216 | E, M, R |
| | organogenesis | 7; 6 | 0.248 | E, M, R |

[symbol]

indicates data missing or illegible when filed

Table 6: the Differently Expressed Genes in Both RRR and RCC Exhibited Distinct Ontologies that are Correlated to RRR Expression Patterns

The functional ontology (Fisher Exact p<0.05) of the differentially expressed genes in both RRR and RCC were crossed compared relative to their expression: concordantly, discordantly, patterns of expression in the current microarray dataset and in terms of Sutton's renal RRR model (Sutton T A et al 2002-FIG. 1), as Initiation (I), extension (E), maintenance (M) and repair (R).

Table 7: The RRR Genes in Non-Probabilistic in-House Ontologies

The comprehensive probabilistic analysis may fail to capture many key aspects of the discordant gene functions. Therefore, we also categorized the genes into gene-by-gene, non-probabilistic in-house ontologies.

### Table 8: Probabilistic Functional Genomics: ARNT Regulated Genes are Enriched for the Concordant Genes and not the Discordant Genes

The two group of genes, the concordantly and discordantly expressed between RRR and RRR, were analyzed for the enrichment in DNA binding elements (based on the Transfac database). One of the elements that was enriched concordant genes and not for the discordant genes is the binding site for the ARNT (HIF-1b dimmer). The up and down denote the genes that were up or down-regulated from normal kidney during RRR or in RCC. The RRR expression (FIG. 3) is indicated as continues, early and late; and the RRR gene expression trend (FIGS. 4, 10). Also indicated if the gene was reported to be regulated by the hetrodimer HIF-1a/ARNT (HRE), hypoxia (H) and Myc pathway (M) (Table 9).

TABLE 8

| | RRR | RRR | RCC | | | |
|---|---|---|---|---|---|---|
| | expression | expression/ | expression/ | Expression | | |
| Symbol | pattern | normal | normal | RRR/RCC | Trend | Notes |
| EMP3 | continues | up | up | concord | 14 | |
| C1QA | continues | up | up | concord | 5 | |
| YWHAH | continues | up | up | concord | 2 | |
| ICAMI | continues | up | up | concord | 2 | H |
| COPEB | continues | up | up | concord | 2 | |
| PTMA | continues | up | up | concord | 2 | M |
| SSR4 | continues | up | up | concord | 6 | |
| TCN2 | continues | down | down | concord | 1 | |
| USP2 | continues | down | down | concord | 1 | |
| CALB1 | continues | down | down | concord | 1 | |
| RPL13A | early | up | up | concord | | |
| MCM7 | early | up | up | concord | 12 | |
| RPS19 | early | up | up | concord | | M |
| MCM4 | early | up | up | concord | 2 | H; M |
| CKS2 | early | up | up | concord | 14 | M |
| KLF5 | early | up | up | concord | 8 | |
| PSMA6 | early | up | up | concord | 2 | M |
| PCBP1 | early | up | up | concord | 8 | |
| FES | early | up | up | concord | 12 | |
| EIF4G2 | early | up | up | concord | 2 | |
| PECI | early | down | down | concord | 3 | |
| DDT | early | down | down | concord | 1 | |
| PIPOX | early | down | down | concord | 3 | |
| GSTT2 | early | down | down | concord | 3 | |
| SELENBP1 | late | down | down | concord | | |
| PSMB10 | late | up | up | concord | | H |
| ITGA6 | late | up | up | concord | 12 | |
| LAPTM5 | late | up | up | concord | 5 | |
| PDGFB | late | up | up | concord | 5 | M |
| PROC | early | down | down | concord | 1 | |
| CORO1B | continues | up | down | discord | 6 | |
| APOE | late | up | down | discord | 5 | |
| KDR | early | down | up | discord | 1 | |
| SCP2 | continues | down | up | discord | 1 | |
| PGK1 | early | down | up | discord | 1 | HRE; H; m |
| EGLN1 | early | down | up | discord | 16 | HRE; H |

### Table 9: The RRR 1325 Genes Expression Data and Specific Functional Gene-Clusters

1325 unique genes were identified in the current microarray dataset. The gene expression is presented as up or down from normal-ischemic kidneys. The genes were further clustered according to RCC vs. normal kidney; renal cell culture hypoxia responsive genes vs. normoxia; HIF regulated genes; VHL, IGF, MYC, NF-kB pathway genes; purine pathway genes; gene expression following renal ischemia reperfusion and/or acute renal failure (ARF) vs. normal tissue; and tissue expression pattern of renal genes (e-renal histology).

### Table 10: An Ontology Analysis in Timely Dependent Fashion: Distinct and Common Ontologies

The differentially expressed genes were clustered according to their pattern of expression as early, late or continually RRR. Functional ontology was analysis performed (Fisher Exact p<0.05). The presented ontologies are the ontology core and are hyperlinked to EMBL-EBI. The average expression of each ontology is presented in a green to red scale; green down-regulated, red up-regulated. See the supplemented Table 10 for a further detailed table

### Table 11: the Differently Expressed Genes in Both RRR and RCC Exhibited Distinct Ontologies for the Concordance Vs Discordance Genes

The differentially expressed genes in both RRR and RCC were clustered according to their concordance Vs discordant change. Functional ontology was analysis performed (Fisher Exact p<0.05). The presented ontologies are the ontology core and are hyperlinked to EMBL-EBI. The average expression of each ontology is presented in a green to red scale; green down-regulated, red unregulated. The number of genes up-/down- regulated in both RRR and RCC is also given and the direction is as in RRR relative to the normal kidney. In terms of Sutton's renal RRR model (Sutton T A et al 2002—FIG. 1) the ontologies are related as extension (E), maintenance (M) and repair (R).

Table 12: the Significance of Gene in the Various Expression Groups: Patterns, Trends and Pathways

The significance of gene in the various expression patterns of early, late, continues, the 27 sub-expression trends, pathways and the concordant or discordant groups was analyzed by using the chi square test (tables 3 and 4). See methods for further explanation.

TABLE 13
An ontology analysis in timely dependent fashion: distinct and common
ontologies. The differentially expressed genes were clustered according to their pattern of
expression as early, late or continually RRR. Functional ontology was analysis performed
(p < 0.05). The presented ontologies are the ontology core and are hyperlinked to EMBL-EBI.
The average RRR expression (log₂) of each ontology is presented in a green to red scale; green
down-regulated, red up-regulated. The numbers and average RRR expression of up- and
down-regulated genes, the category p-value and enrichment are shown as well.

| Early(A)/ | | Early (A) | | | | | |
|---|---|---|---|---|---|---|---|
| Late(B)/ | | | Total | | Total | | |
| Continuous | Ontology | Average | Expression | No Genes | Expression | No Genes | |
| (*) | Category | Expression | UP | UP | DOWN | DOWN | p < 0.05 |
| Early (A) | ATP-binding and | −0.477 | 0 | 0 | −1.4296857 | 3 | 0.021897 |
| | phosphorylation- | | | | | | |
| | dependent chloride | | | | | | |
| | channel activity | | | | | | |
| | intramolecular | −0.723 | 0 | 0 | −3.6167037 | 5 | 0.003126 |
| | isomerase activity\, | | | | | | |
| | transposing C═C | | | | | | |
| | bonds | | | | | | |
| | cis-trans isomerase | 0.169 | 1.8976128 | 4 | −0.8812236 | 2 | 0.01318 |
| | activity | | | | | | |
| | growth factor binding | −0.452 | 0.383383 | 1 | −3.0957649 | 5 | 0.021394 |
| | peptidyl-prolyl cis- | 0.335 | 1.8976128 | 4 | −0.2247992 | 1 | 0.046163 |
| | trans isomerase | | | | | | |
| | activity | | | | | | |
| | intramolecular | −0.533 | 0.4166733 | 1 | −3.6167037 | 5 | 0.032366 |
| | isomerase activity | | | | | | |
| | transferase activity\, | 0.032 | 2.0043726 | 4 | −1.7833621 | 3 | 0.022759 |
| | transferring alkyl or | | | | | | |
| | aryl (other than | | | | | | |
| | methyl) groups | | | | | | |
| | heat shock protein | 0.345 | 2.5901036 | 5 | −0.5213829 | 1 | 0.046307 |
| | activity | | | | | | |
| | isomerase activity | −0.181 | 2.6834421 | 6 | −5.5739205 | 10 | 0.000394 |
| | lyase activity | −0.218 | 2.4797409 | 5 | −5.7457532 | 10 | 0.000916 |
| | hydrogen ion | −0.441 | 0 | 0 | −4.408021 | 10 | 0.032021 |
| | transporter activity | | | | | | |
| | magnesium ion | −0.144 | 1.4708483 | 3 | −3.0511803 | 8 | 0.028411 |
| | binding | | | | | | |
| | monovalent inorganic | −0.441 | 0 | 0 | −4.408021 | 10 | 0.03994 |
| | cation transporter | | | | | | |
| | activity | | | | | | |
| | electron transporter | −0.023 | 2.8000896 | 6 | −3.1018422 | 7 | 0.04598 |
| | activity | | | | | | |
| | carrier activity | −0.289 | 4.0621543 | 8 | −12.165679 | 20 | 0.023625 |
| | transferase activity | 0.097 | 19.074923 | 42 | −12.687227 | 24 | 0.027974 |
| | catalytic activity | 0.025 | 53.199976 | 116 | −48.079162 | 93 | 7.09E−05 |
| | proton-transporting | −0.422 | 0 | 0 | −1.6880515 | 4 | 0.024764 |
| | two-sector ATPase | | | | | | |
| | complex | | | | | | |
| | hydrogen- | −0.422 | 0 | 0 | −1.6880515 | 4 | 0.024764 |
| | translocating F-type | | | | | | |
| | ATPase complex | | | | | | |
| | inner membrane | −0.338 | 0.6451115 | 2 | −4.7047745 | 10 | 0.019819 |
| | extrachromsomal | −0.195 | 1.9705466 | 5 | −4.50828 | 8 | 0.033456 |
| | circular DNA | | | | | | |
| | extrachromosomal | −0.195 | 1.9705466 | 5 | −4.50828 | 8 | 0.033456 |
| | DNA | | | | | | |
| | endoplasmic | −0.011 | 6.2680131 | 17 | −6.5718272 | 10 | 0.049052 |
| | reticulum | | | | | | |
| | cytoplasm | 0.049 | 53.881622 | 110 | −44.500056 | 83 | 0.004815 |
| | intracellular | 0.10 | 83.220823 | 174 | −55.152258 | 107 | 0.002094 |
| | oxidative | −0.417 | 0 | 0 | −1.6664665 | 4 | 0.017917 |
| | phosphorylation | | | | | | |
| | DNA replication | 0.626 | 3.7557997 | 6 | 0 | 0 | 0.001496 |
| | initiation | | | | | | |
| | fatty acid oxidation | −0.822 | 0 | 0 | −3.2874914 | 4 | 0.037675 |
| | sulfur amino acid | −0.589 | 0.2312001 | 1 | −2.5888117 | 3 | 0.050404 |
| | metabolism | | | | | | |
| | DNA dependent | 0.446 | 5.1596519 | 10 | −0.2508499 | 1 | 7.45E−05 |
| | DNA replication | | | | | | |
| | response to | 0.256 | 2.4665696 | 4 | −0.9325186 | 2 | 0.016593 |
| | temperature | | | | | | |
| | response to heat | 0.389 | 2.4665696 | 4 | −0.5213829 | 1 | 0.045385 |
| | glycolysis | −0.161 | 0.8571094 | 2 | −2.1445047 | 6 | 0.005719 |
| | glucose metabolism | −0.351 | 0.8571094 | 2 | −5.4201862 | 11 | 0.000218 |
| | regulation of | 0.004 | 1.3317573 | 4 | −1.3056009 | 3 | 0.015072 |
| | translation | | | | | | |
| | nucleoside | −0.111 | 1.0236657 | 2 | −1.6880515 | 4 | 0.031704 |
| | triphosphate | | | | | | |
| | metabolism | | | | | | |
| | monosaccharide | −0.161 | 0.8571094 | 2 | −2.1445047 | 6 | 0.010791 |
| | catabolism | | | | | | |
| | alcohol catabolism | −0.161 | 0.8571094 | 2 | −2.1445047 | 6 | 0.010791 |
| | glucose catabolism | −0.161 | 0.8571094 | 2 | −2.1445047 | 6 | 0.010791 |
| | hexose catabolism | −0.161 | 0.8571094 | 2 | −2.1445047 | 6 | 0.010791 |
| | protein-nucleus | 0.530 | 3.7114818 | 7 | 0 | 0 | 0.026516 |
| | import | | | | | | |
| | amine biosynthesis | −0.338 | 1.0005872 | 2 | −3.3664601 | 5 | 0.026516 |
| | monosaccharide | −0.378 | 0.8571094 | 2 | −6.1543298 | 12 | 0.00071 |
| | metabolism | | | | | | |
| | hexose metabolism | −0.351 | 0.8571094 | 2 | −5.4201862 | 11 | 0.00169 |
| | S phase of mitotic | 0.384 | 6.8410074 | 14 | −0.6972074 | 2 | 0.000442 |
| | cell cycle | | | | | | |
| | DNA replication | 0.384 | 6.8410074 | 14 | −0.6972074 | 2 | 0.000442 |
| | main pathways of | −0.256 | 0.8571094 | 2 | −3.925259 | 10 | 0.003322 |
| | carbohydrate | | | | | | |
| | metabolism | | | | | | |
| | carbohydrate | −0.161 | 0.8571094 | 2 | −2.1445047 | 6 | 0.029502 |
| | catabolism | | | | | | |
| | energy derivation by | −0.323 | 1.4198075 | 3 | −6.257679 | 12 | 0.002202 |
| | oxidation of organic | | | | | | |
| | compounds | | | | | | |
| | DNA replication and | 0.378 | 7.1267635 | 15 | −0.6972074 | 2 | 0.001282 |
| | chromosome cycle | | | | | | |
| | energy pathways | −0.359 | 1.4198075 | 3 | −7.5263925 | 14 | 0.001924 |
| | mitotic cell cycle | 0.457 | 15.101651 | 28 | −0.9305031 | 3 | 2.17E−05 |
| | coenzyme | −0.513 | 0.3028057 | 1 | −5.4314898 | 9 | 0.034759 |
| | metabolism | | | | | | |
| | protein folding | 0.398 | 4.5118926 | 8 | −0.5365947 | 2 | 0.043069 |
| | alcohol metabolism | −0.346 | 1.1939183 | 3 | −7.0708879 | 14 | 0.009441 |
| | coenzyme and | −0.381 | 1.2459281 | 2 | −5.4314898 | 9 | 0.045605 |
| | prosthetic group | | | | | | |
| | metabolism | | | | | | |
| | DNA metabolism | 0.386 | 16.863937 | 33 | −2.1852938 | 5 | 9.41E−05 |
| | carbohydrate | −0.240 | 3.1254157 | 8 | −9.1279893 | 17 | 0.003907 |
| | metabolism | | | | | | |
| | cell cycle | 0.436 | 20.308961 | 40 | −1.1459049 | 4 | 0.009025 |
| | cell proliferation | 0.393 | 26.171638 | 49 | −3.7762005 | 8 | 0.008789 |
| | cell growth and/or | 0.136 | 53.452631 | 102 | −31.309554 | 61 | 0.003237 |
| | maintenance | | | | | | |
| | metabolism | 0.096 | 77.803497 | 165 | −52.569002 | 98 | 0.001322 |
| Continues | oxidoreductase | −0.336 | 5.211 | 11 | −17.994 | 27 | 0.0113 |
| (*) and | activity | | | | | | |
| Early(A) | mitochondrion | −0.379 | 2.9873 | 8 | −19.276 | 35 | 0.0018 |
| | cytosol | 0.312 | 10.557 | 21 | −2.4344 | 5 | 0.0264 |
| | fatty acid metabolism | −0.537 | 0.7428 | 2 | −6.6505 | 9 | 0.0415 |
| | carboxylic acid | −0.509 | 1.4427 | 4 | −14.162 | 21 | 0.0093 |
| | metabolism | | | | | | |
| | organic acid | −0.509 | 1.4427 | 4 | −14.162 | 21 | 0.01 |
| | metabolism | | | | | | |
| | biosynthesis | 0.043 | 16.388 | 31 | −13.952 | 25 | 0.0022 |
| | macromolecule | 0.134 | 14.8 | 28 | −8.7637 | 17 | 0.0148 |
| | biosynthesis | | | | | | |
| | physiological process | 0.105 | 111.7 | 224 | −73.559 | 139 | 0.0049 |
| Early(A)/ | | | | | | | |
| Late(B)/ | | | Total | | Total | | |
| Continuous | | Average | Expression | No Genes | Expression | No Genes | |
| (*) | Category | Expression | UP | UP | DOWN | DOWN | p < 0.05 |
| Continues | oxidoreductase | −0.531 | 4.3187 | 7 | −20.252 | 23 | 0.0004 |
| (*) and | activity | | | | | | |
| Early(A) | mitochondrion | −0.590 | 1.3594 | 3 | −16.12 | 22 | 0.0205 |
| | cytosol | 0.410 | 11.692 | 15 | −3.0865 | 6 | 0.0015 |
| | fatty acid metabolism | −0.530 | 1.2748 | 2 | −8.6969 | 2 | 0.00001 |
| | carboxylic acid | −0.608 | 1.8196 | 3 | −18.231 | 24 | 4E−07 |
| | metabolism | | | | | | |
| | organic acid | −0.608 | 1.8196 | 3 | −18.231 | 24 | 4E−07 |
| | metabolism | | | | | | |
| | biosynthesis | 0.223 | 18.016 | 24 | −10.207 | 11 | 0.0099 |
| | macromolecule | 0.413 | 18.016 | 24 | −5.6193 | 6 | 0.0144 |
| | biosynthesis | | | | | | |
| | physiological process | 0.125 | 103.31 | 134 | −75.551 | 88 | 0.0051 |
| Continues | defense response | 0.696 | 16.7006662 | 24 | 0 | 0 | 0.039612 |
| (*) and | response to biotic | 0.581 | 16.7006662 | 24 | −1.594032 | 2 | 0.033838 |
| Late(B) | stimulus | | | | | | |
| | response to external | 0.493 | 21.7840142 | 30 | −4.0365428 | 6 | 0.007599 |
| | stimulus | | | | | | |
| | extracelluar space | 0.248 | 39.566685 | 49 | −21.740572 | 23 | 0.004952 |
| Continuous | L-phenylalanine | −1.203 | 0 | 0 | −3.6084015 | 3 | 0.015458 |
| (*) | metabolism | | | | | | |
| | phenylalanine | −1.203 | 0 | 0 | −3.6084015 | 3 | 0.015458 |
| | catabolism | | | | | | |
| | aromatic amino acid | −1.203 | 0 | 0 | −3.6084015 | 3 | 0.024874 |
| | family catabolism | | | | | | |
| | aromatic compound | −1.203 | 0 | 0 | −3.6084015 | 3 | 0.024874 |
| | catabolism | | | | | | |
| | immunoglobulin | 1.103 | 3.30923671 | 3 | 0 | 0 | 0.035077 |
| | binding | | | | | | |
| | cytosolic ribosome | 0.823 | 9.87532021 | 12 | 0 | 0 | 2.15E−08 |
| | (sensu Eukarya) | | | | | | |
| | eukaryotic 48S | 0.749 | 2.9978872 | 4 | 0 | 0 | 0.007969 |
| | initiation complex | | | | | | |
| | cytosolic small | 0.749 | 2.9978872 | 4 | 0 | 0 | 0.007969 |
| | ribosomal subunit | | | | | | |
| | (sensu Eukarya) | | | | | | |
| | eukaryotic 43S | 0.688 | 3.43951302 | 5 | 0 | 0 | 0.005113 |
| | preinitiation complex | | | | | | |
| | amino acid | −0.940 | 0 | 0 | −5.639126 | 6 | 0.002465 |
| | catabolism | | | | | | |
| | amine catabolism | −0.940 | 0 | 0 | −5.639126 | 6 | 0.003956 |
| | actin filament | 0.340 | 2.02074983 | 3 | −0.6610948 | 1 | 0.034693 |
| | small ribosomal | 0.746 | 3.73192432 | 5 | 0 | 0 | 0.014953 |
| | subunit | | | | | | |
| | ribosome biogenesis | 0.872 | 8.71636391 | 10 | 0 | 0 | 0.000176 |
| | ribosome biogenesis | 0.872 | 8.71636391 | 10 | 0 | 0 | 0.000215 |
| | and assembly | | | | | | |
| | anion transporter | −0.381 | 0.86455186 | 1 | −2.7709958 | 4 | 0.024795 |
| | activity | | | | | | |
| | inorganic anion | 0.283 | 2.54243996 | 3 | −1.1252084 | 2 | 0.030187 |
| | transport | | | | | | |
| | aromatic compound | −0.396 | 2.14211399 | 2 | −5.3088476 | 6 | 0.003206 |
| | metabolism | | | | | | |
| | structural constituent | 0.799 | 15.9701069 | 20 | 0 | 0 | 5.05E−07 |
| | of ribosome | | | | | | |
| | chemokine receptor | 0.903 | 4.51414395 | 5 | 0 | 0 | 0.04313 |
| | binding | | | | | | |
| | G-protein-coupled | 0.903 | 4.51414395 | 5 | 0 | 0 | 0.04313 |
| | receptor binding | | | | | | |
| | chemokine activity | 0.903 | 4.51414395 | 5 | 0 | 0 | 0.04313 |
| | posttranslational | −0.049 | 2.61952085 | 4 | −2.9596796 | 3 | 0.013421 |
| | membrane targeting | | | | | | |
| | basement membrane | 0.991 | 4.95649472 | 5 | 0 | 0 | 0.051961 |
| | ribosome | 0.786 | 16.5148623 | 21 | 0 | 0 | 1.5E−06 |
| | blood coagulation | 0.419 | 4.82540533 | 6 | −1.4758496 | 2 | 0.007437 |
| | hemostasis | 0.419 | 4.82540533 | 6 | −1.4758496 | 2 | 0.0095 |
| | heparin binding | 0.342 | 3.84657601 | 4 | −1.7921275 | 2 | 0.044879 |
| | protein-ER targeting | −0.049 | 2.61952085 | 4 | −2.9596796 | 3 | 0.026414 |
| | anion transport | −0.033 | 2.54243996 | 3 | −2.7709958 | 4 | 0.026414 |
| | protein-membrane | −0.049 | 2.61952085 | 4 | −2.9596796 | 3 | 0.026414 |
| | targeting | | | | | | |
| | chemotaxis | 0.845 | 5.91347974 | 7 | 0 | 0 | 0.038606 |
| | taxis | 0.845 | 5.91347974 | 7 | 0 | 0 | 0.038606 |
| | ribonucleoprotein | 0.764 | 19.0966734 | 25 | 0 | 0 | 1.68E−05 |
| | complex | | | | | | |
| | actin binding | 0.177 | 4.89579982 | 8 | −2.9470927 | 3 | 0.012932 |
| | response to chemical | 0.610 | 7.13862643 | 9 | −1.0401916 | 1 | 0.02206 |
| | substance | | | | | | |
| | amino acid | −0.695 | 0.5447554 | 1 | −7.4931106 | 9 | 0.025541 |
| | metabolism | | | | | | |
| | structural molecule | 0.849 | 30.5748631 | 36 | 0 | 0 | 6.36E−06 |
| | activity | | | | | | |
| | amino acid and | −0.755 | 0.5447554 | 1 | −9.6036406 | 11 | 0.021417 |
| | derivative metabolism | | | | | | |
| | response to abiotic | 0.472 | 9.99208761 | 12 | −2.4425107 | 4 | 0.011197 |
| | stimulus | | | | | | |
| | cytoplasm | 0.736 | 19.5172428 | 23 | −1.1062014 | 2 | 0.001275 |
| | organization and | | | | | | |
| | biogenesis | | | | | | |
| | ion transporter | −0.561 | 1.42337687 | 2 | −8.1543369 | 10 | 0.035369 |
| | activity | | | | | | |
| | amine metabolism | −0.755 | 0.5447554 | 1 | −9.6036406 | 11 | 0.047678 |
| | protein biosynthesis | 0.772 | 16.2160128 | 21 | 0 | 0 | 0.012248 |
| | RNA binding | 0.606 | 13.1020883 | 17 | −1.5930626 | 2 | 0.019029 |
| | cell organization and | 0.723 | 21.3449184 | 26 | −1.1062014 | 2 | 0.010322 |
| | biogenesis | | | | | | |
| | extracellular | 0.283 | 43.5375175 | 54 | −21.740572 | 23 | 0.009792 |

| Early(A)/ | | Late (B) | | | | | | |
|---|---|---|---|---|---|---|---|---|
| Late(B)/ | | | Total | No | Total | No | | |
| Continuous | Ontology | Average | Expression | Genes | Expression | Genes | | |
| (*) | Category | Expression | UP | UP | DOWN | DOWN | p < 0.05 | Enrichment |
| Late (B) | urea cycle | 0.244 | 1.130631 | 2 | −0.39848 | 1 | 0.0157 | 14.066206 |
| | intermediate | | | | | | | |
| | metabolism | | | | | | | |
| | MHC class I receptor | 0.765 | 2.295813 | 3 | 0 | 0 | 0.02366 | 11.645783 |
| | activity | | | | | | | |
| | antigen processing\, | 0.765 | 2.295813 | 3 | 0 | 0 | 0.02525 | 11.252964 |
| | endogenous antigen | | | | | | | |
| | via MHC class I | | | | | | | |
| | antigen presentation\, | 0.765 | 2.295813 | 3 | 0 | 0 | 0.02525 | 11.252964 |
| | endogenous antigen | | | | | | | |
| | collagenase activity | 0.877 | 2.629886 | 3 | 0 | 0 | 0.0343 | 9.7048193 |
| | phospholipase | 0.893 | 2.679154 | 3 | 0 | 0 | 0.0343 | 9.7048193 |
| | inhibitor activity | | | | | | | |
| | antigen presentation | 1.021 | 7.147112 | 7 | 0 | 0 | 4.4E−05 | 9.3774704 |
| | antigen processing | 1.122 | 6.732498 | 6 | 0 | 0 | 0.00037 | 8.6561265 |
| | hydrolase activity\, | 0.518 | 1.55403 | 3 | 0 | 0 | 0.04642 | 8.3184165 |
| | acting on carbon- | | | | | | | |
| | nitrogen (but not | | | | | | | |
| | peptide) bonds\, in | | | | | | | |
| | linear amidines | | | | | | | |
| | proteasome core | 0.594 | 2.377945 | 4 | 0 | 0 | 0.03453 | 5.3784861 |
| | complex (sensu | | | | | | | |
| | Eukarya) | | | | | | | |
| | apoptosis inhibitor | 0.489 | 2.446018 | 5 | 0 | 0 | 0.03658 | 3.8819277 |
| | activity | | | | | | | |
| | hydrolase activity\, | 0.484 | 2.904975 | 6 | 0 | 0 | 0.0473 | 2.9860982 |
| | acting on carbon- | | | | | | | |
| | nitrogen (but not | | | | | | | |
| | peptide) bonds | | | | | | | |
| | immune response | 0.779 | 27.7517 | 30 | −2.03277 | 3 | 8.2E−07 | 2.5788043 |
| | apoptosis regulator | 0.496 | 3.966895 | 8 | 0 | 0 | 0.05082 | 2.3526835 |
| | activity | | | | | | | |
| | response to | 0.732 | 14.8756 | 16 | −1.69189 | 2 | 0.00157 | 2.3281995 |
| | pest/pathogen/parasite | | | | | | | |
| | response to wounding | 0.395 | 6.433227 | 10 | −1.69189 | 2 | 0.01308 | 2.3201989 |
| | extracellular matrix | 0.844 | 13.51148 | 16 | 0 | 0 | 0.01161 | 2.0214444 |
| | transmembrane | 0.677 | 16.22933 | 21 | −0.66253 | 2 | 0.01162 | 1.7370494 |
| | receptor activity | | | | | | | |
| | peptidase activity | 0.464 | 10.75818 | 19 | −1.01553 | 2 | 0.03044 | 1.6304096 |
| | response to stress | 0.540 | 16.76545 | 20 | −3.267 | 5 | 0.04162 | 1.4979985 |
| | integral to plasma | 0.305 | 12.9202 | 17 | −4.98278 | 9 | 0.04397 | 1.4742236 |
| | membrane | | | | | | | |
| | receptor activity | 0.516 | 21.37252 | 32 | −2.26642 | 5 | 0.02041 | 1.4391916 |
| | signal transducer | 0.428 | 29.10036 | 46 | −5.14292 | 10 | 0.01616 | 1.332034 |
| | activity | | | | | | | |
| Continues | defense response | 0.788 | 29.62142 | 32 | −2.03277 | 3 | 1.3E−05 | 2.2027615 |
| (*) and | response to biotic | 0.743 | 30.79255 | 34 | −2.57173 | 4 | 5.4E−06 | 2.1928854 |
| Late(B) | stimulus | | | | | | | |
| | response to external | 0.607 | 31.1322 | 35 | −5.01693 | 8 | 9E−05 | 1.8370443 |
| | stimulus | | | | | | | |
| | extracellular space | 0.692 | 53.45553 | 65 | −4.34795 | 6 | 0.03805 | 1.2228305 |

### Table 14:

The differential gene expressions clustered into 27 trends in a timely dependent fashion, three of which were singletons. For each gene, the data is presented in fold ratios from the normal genes expression across the whole RRR period, with the gene identifiers. Highlighted in gray are the pattern identification number, and gene symbol.

### Table 15: Molecular Drug Targets Found Among the Concordantly Expressed Genes.

The genes expressed concordantly between RRR and RCC were used to search for known Molecular drug targets. Listed are the concordant gene symbol, the expression in RRR and RCC relative to normal kidney, the actual gene that is targeted by the drug, is the targeted gene is a concordant gene or in its pathway, manufacturer, generic name of the drug, the world status of the drug (no development reported, discontinued, preclinical, Phase I-III Clinical Trials, launched and fully launched) and the drug therapy description.

### Table 16: Molecular Diagnostic Markers Among the Discordantly Expressed Genes.

Out of all the discordant genes, three genes, FHIT, KDR and VEGF were reported in diagnostic immunohistochemistry of clinical samples of various pathologies. Further information is available at Linscott's Directory (http://www.linscottsdirectory.com) and ImmunoQuery (http://www.immunoquery.com).

TABLE 21
Pathway analysis of genes differentially expressed in RRR and RCC.

| RRR + RCC | RRR + RCC | RRR + RCC |
|---|---|---|
| All genes | Concordanat | Discordant |
| VHL | VHL | VHL |
| Hypoxia | Hypoxia | Hypoxia |
| HIF (HRE) | | HIF (HRE) |
| IGF | | IGF |
| MYC | MYC | |
| p53 | p53 | p53 |
| NF-κB | NF-κB | |

Genes differentially expressed on both RRR and RCC were analyzed for significant enrichment (p<0.05) in genes belonging to VHL, hypoxia, HRE, IGF1, MYC, p53 and NF-κB pathways. The RRR genes were not filtered by phases of expression (i.e., continuous, early and late; further details are given in Table 18).

### Table 22. Gene Ontology Analysis of Concordant and Discordant Genes in RRR and RCC

GO categories enriched in concordant or discordant genes in RRR and RCC are shown. The average log₂ change in gene expression for genes associated with each category is shown. Red and green shading indicate up- and down-regulated genes, respectively (further details are given in Table 17).

TABLE 22

| | | | | |
|---|---|---|---|---|
| | | | GO term | |
| | | # Genes | average fold | Category |
| GO System | GO term | UP/DOWN | change | enrichment |

Concordant expression

| | | | | |
|---|---|---|---|---|
| Molecular Function | Immunoglobulin binding | 3; 0 | [symbol] | 9.7 |
| | structural constituent of ribosome | 24; 0 | [symbol] | 4.7 |
| | RNA binding | 27; 1 | [symbol] | 2.7 |
| | extracellular matrix structural constituen | 6; 0 | [symbol] | 3.1 |
| Cellular Component | cytosolic ribosome | 11; 0 | [symbol] | 8.1 |
| | proteasome core complex | 4; 0 | [symbol] | 5.6 |
| | collagen | 5; 0 | [symbol] | 4.9 |
| | extracellular matrix | 13; 1 | [symbol] | 1.9 |
| Biological Process | DNA replication initiation | 5; 0 | [symbol] | 8.6 |
| | regulation of transiation | 4; 2 | 0.187 | 4.8 |
| | ribosome biogenesis | 10; 0 | [symbol] | 4.8 |
| | posttranslational membrane targeting | 5; 2 | 0.491 | 3.5 |
| | cytoplasm organization and biogenesis* | 20; 2 | [symbol] | 1.8 |
| | macromolecule biosynthesis | 29.3 | [symbol] | 1.7 |
| | cell adhesion | 19.2 | [symbol] | 1.7 |
| | immune response | 21.0 | [symbol] | 1.7 |
| | cell growth and/or maintenance* | 78; 25 | [symbol] | 1.3 |
| | protein metabolism | 60; 10 | [symbol] | 1.3 |
| | protein-ER targeting | 6; 2 | [symbol] | 3.5 |
| | cell proliferation | 33; 1 | [symbol] | 1.4 |

Discordant expression

| | | | | |
|---|---|---|---|---|
| Molecular Function | insulin-like growth factor binding | 2; 2 | 0.088 | 21.5 |
| | organic cation transporter activity | 1; 2 | 0.268 | 14.9 |
| | heparin binding | 4; 2 | 0.253 | 10.2 |
| | catalytic activity | 9; 30 | [symbol] | 1.3 |
| Cellular Component | extracellular space | 12; 12 | 0.085 | 1.5 |
| Biological Process | one-carbon compound metabolism | 0; 3 | [symbol] | 11 |
| | angiogenesis | 3; 2 | 0.392 | 8.7 |
| | regulation of cell growth | 2; 2 | 0.088 | 8.3 |
| | cytoskeleton organization and biogenesis | 5; 3 | 0.194 | 3.2 |
| | cytoplasm organization and biogenesis* | 5; 4 | 0.105 | 2.4 |
| | morphogenesis | 8; 6 | 0.288 | 1.7 |
| | cell growth and/or maintenance* | 13; 20 | 0.127 | 1.3 |

[symbol]

indicates data missing or illegible when filed

TABLE 23
Classification of discordant genes by functional category based on extensive
analysis of the RRR and RCC literatures.

| Category | Regeneration | RCC | Gene Symbol |
|---|---|---|---|
| Morphogenesis | Up | Down | CRYM; CTGF; GPC3; CYR61; MYL6; TCF21; THBS1 |
| | Down | Up | FHL1; KDR; PKD1; RTN3; VEGF; GADD45G |
| Extracelluler space | Up | Down | APOE; IF; DCN; CTGF; GC; GPC3; CYR61; MMP2; PLAT; SDC1; THBS1; TACSTD2 |
| | Down | Up | BCKDHA; CD59; COX6C; IGFBP1; IGFBP3; KDR; Klk1; LPL; MEP1A; ENPP2; |
| | | | RTN3; VEGF |
| Metabolism | Up | Down | APOE; CTGF/IGFBP8 |
| | Down | Up | BCKDHA; AMACR; ENPP2; MTHFD1; MAT2A; SHMT2; SPTLC1; LPL; SHMT1; |
| | | | PTPRB; SOD2; CPT1A; ACOX1; EGLN1 |
| Glycolysis | Up | Down | |
| | Down | Up | PGK1; HK1 |
| Signal transduction | Up | Down | SAR1; RALBP1; NR2F6; SMC1L1; TACSTD2 |
| | Down | Up | IGFBP1; IGFBP3; ARHE; PCTK3; VEGF; CD59; FRAP1 |
| Angiogenesis | Up | Down | CTGF; CYR61; THBS1 |
| | Down | Up | VEGF; KDR |
| Transcription | Up | Down | TCF21; ZNF144; NR2F6 |
| | Down | Up | GRSF1; NCOA4; PAPOLA; UBE2V1; EIF4A2; MKNK2; SOD2 |
| Transport | Up | Down | GC; SLC1A1; APOE; SAR1; RALBP1 |
| | Down | Up | SCP2; SLC16A7; GJB2; ATP1B1; COX6C; SLC22A1; CPT1A; ACOX1; ARHE |
| Proteolysis | Up | Down | IF; PLAT |
| | Down | Up | Klk1; MEP1A |
| Immune | Up | Down | |
| | Down | Up | CEACAM1; CD59 |
| DNA | Up | Down | SMC1L1; CTGF/IGFBP8 |
| | Down | Up | TOP3B; RRM1; GADD45G; FRAP1 |
| Cell adhesion | Up | Down | THBS1; CTGF/IGFBP8; CYR61/IGFBP10 |
| | Down | Up | PKD1 |
| Cell differentiation | Up | Down | |
| | Down | Up | FHL1; GADD45G |
| Do/phosphorylation | Up | Down | PTPRO; PPP2CB; |
| | Down | Up | PTPRB; PCTK3; MKNK2; KDR |
| Ubiquitination | Up | Down | ZNF144 |
| | Down | Up | UBE2V1; EGLN1 |
| Others | Up | Down | TJP2; MT2A; TM4SF3; SDC1; CORO1B; WSB1; MYL6; AKAP2; CRYM; DCN |
| | Down | Up | HARS; C16orf5; RTN3; KIAA1049; HSPH1; KIF21A; ADD3; HSPD1; CAPNS1 |

TABLE 2

| | | | Late Pattern: | Continues | | |
|---|---|---|---|---|---|---|
| | | Early Pattern: | Category | Pattern: | | |
| | | Category | Average | Category | | |
| | | Average | Expression | Average | | |
| | | Expression | (RRR | Expression | No | No |
| | | (RRR phases: I, | phases: M, | (RRR phases: | Genes | Genes |
| Go System | Category | E, early M) | R) | I, E, M, R) | UP | DOWN |
| Molecular | ATP-binding and phosphorylation- | −0.477 | | | 0 | 3 |
| Function | dependent chloride channel activity | | | | | |
| | cyclophilin-type peptidy-prolyl cis-trans | 0.336 | | | 4 | 1 |
| | isomerase activity | | | | | |
| | cis-trans isomerase activity | 0.170 | | | 4 | 2 |
| | intramolecular isomerase activity | −0.533 | | | 1 | 5 |
| | growth factor binding | −0.453 | | | 1 | 5 |
| | transferase activity\, transferring alkyl or | 0.031 | | | 4 | 3 |
| | aryl (other than methyl) groups | | | | | |
| | lyase activity | −0.218 | | | 5 | 10 |
| | isomerase activity | −0.217 | | | 5 | 10 |
| | hydrogen ion transporter activity | −0.441 | | | 0 | 10 |
| | magnesium ion binding | −0.199 | | | 2 | 8 |
| | monovalent inorganic cation transporter | −0.441 | | | 0 | 10 |
| | activity | | | | | |
| | carrier activity | −0.326 | | | 7 | 21 |
| | oxidoreductase activity | −0.377 | | −0.573 | 9; 6 | 26; 22 |
| | MHC class I receptor activity | | 0.767 | | 3 | 0 |
| | collagenase activity | | 0.877 | | 3 | 0 |
| | phospholipase inhibitor activity | | 0.897 | | 3 | 0 |
| | hydrolase activity\, acting on carbon- | | 0.517 | | 3 | 0 |
| | nitrogen (but not peptide) bonds\, in | | | | | |
| | linear amidines | | | | | |
| | apoptosis inhibitor activity | | 0.486 | | 5 | 0 |
| | immunoglobulin binding | | | 1.103 | 3 | 0 |
| | anion transporter activity | | | −0.384 | 1 | 4 |
| | structural constituent of ribosome | | | 0.798 | 20 | 0 |
| | chemokine activity | | | 0.902 | 5 | 0 |
| | actin binding | | | 0.176 | 8 | 3 |
| | structural constituent of cytoskeleton | | | 0.968 | 8 | 0 |
| | RNA binding | | | 0.605 | 17 | 2 |
| Cellular | hydrogen-translocating F-type ATPase | −0.423 | | | 0 | 4 |
| Component | complex | | | | | |
| | mitochondrial inner membrane | −0.371 | | | 2 | 9 |
| | extrachromosomal DNA | −0.194 | | | 5 | 8 |
| | cytoplasm | 0.059 | | | 118 | 84 |
| | mitochondrion | −0.393 | | −0.590 | 8; 3 | 35; 22 |
| | cytosol | 0.340 | | 0.410 | 21; 15 | 4; 6 |
| | proteasome core complex (sensu | | 0.595 | | 4 | 0 |
| | Eukarya) | | | | | |
| | microfibril | | 1.296 | | 7 | 0 |
| | extracellular space | | 0.664 | 0.247 | 64; 49 | 8; 23 |
| | cytosolic ribosome (sensu Eukarya) | | | 0.823 | 12 | 0 |
| | cytosolic small ribosomal subunit (sensu | | | 0.750 | 4 | 0 |
| | Eukarya) | | | | | |
| | small ribosomal subunit | | | 0.746 | 5 | 0 |
| | actin filament | | | 0.340 | 3 | 1 |
| | extracellular | | | 0.282 | 54 | 23 |
| iological | oxidative phosphorylation | −0.418 | | | 0 | 4 |
| rocess | DNA replication initiation | 0.692 | | | 5 | 0 |
| | regulation of translation | 0.003 | | | 4 | 3 |
| | group transfer coenzyme metabolism | −0.452 | | | 0 | 5 |
| | ribonucleoside triphosphate biosynthesis | −0.256 | | | 1 | 4 |
| | purine ribonucleoside triphosphate | −0.256 | | | 1 | 4 |
| | biosynthesis | | | | | |
| | glycolysis | −0.163 | | | 2 | 6 |
| | S phase of mitotic cell cycle | 0.389 | | | 12 | 2 |
| | fatty acid metabolism | −0.550 | | −0.523 | 2; 2 | 8; 10 |
| | biosynthesis | 0.051 | | 0.223 | 30; 24 | 23; 11 |
| | urea cycle intermediate metabolism | | 0.243 | | 2 | 1 |
| | antigen presentation\, endogenous | | 0.767 | | 3 | 0 |
| | antigen | | | | | |
| | antigen processing\, endogenous antigen | | 0.767 | | 3 | 0 |
| | via MHC class I | | | | | |
| | response to wounding | | 0.384 | | 8 | 2 |
| | response to pest/pathogen/parasite | | 0.791 | | 13 | 2 |
| | catabolism | | 0.526 | | 25 | 3 |
| | defense response | | 0.849 | 0.696 | 26; 24 | 3; 0 |
| | phenylalanine catabolism | | | −1.203 | 0 | 3 |
| | amino acid biosynthesis | | | −0.873 | 0 | 4 |
| | ribosome biogenesis | | | 0.872 | 10 | 0 |
| | inorganic anion transport | | | 0.282 | 3 | 2 |
| | aromatic compound metabolism | | | −0.366 | 2 | 5 |
| | posttranslational membrane targeting | | | −0.049 | 4 | 3 |
| | blood coagulation | | | 0.340 | 5 | 2 |
| | anion transport | | | −0.034 | 3 | 4 |
| | ER organization and biogenesis | | | −0.049 | 4 | 3 |
| | amino acid metabolism | | | −0.721 | 1 | 8 |
| | response to chemical substance | | | 0.564 | 8 | 1 |
| | cytoplasm organization and biogenesis | | | 0.543 | 26 | 5 |
| | macromolecule biosynthesis | | | 0.771 | 21 | 0 |
| | protein biosynthesis | | | 0.771 | 21 | 0 |
| | organelle organization and biogenesis | | | 0.387 | 16 | 5 |

TABLE 4

| | | | | | | | | |
|---|---|---|---|---|---|---|---|---|
| | | | No. of | | | | % of all | |
| | | | genes that | No. of genes that | % of all the 361 | In a category: the % of | the category | |
| | | Category | are changed | are changed on | genes changed on | renal regeneration genes | that is changed on | |
| | | size (No. | in renal | both renal | both renal | that are changed on both | both renal | |
| | | of genes) | regeneration | regeneration | regeneration and | renal regeneration and | regeneration and | |
| No. | Category name | (A) | (B) | and RCC (C) | RCC | RCC (C/B) | RCC (C/A) | p value |

A. All genes changed in both renal regeneration and RCC:

| | | | | | | | | |
|---|---|---|---|---|---|---|---|---|
| 1 | RCC | 984 | 361 | 361 | 100 | 100 | 37 | <0.00001 |
| 2 | VHL pathway | 282 | 104 | 75 | 21 | 72 | 27 | <0.00001 |
| 3 | Hypoxia pathway | 251 | 95 | 51 | 14 | 54 | 20 | <0.00001 |
| 4 | HRE target (HIF) | 39 | 17 | 11 | 3 | 65 | 28 | <0.0001 |
| 5 | IGF pathway | 139 | 37 | 17 | 5 | 46 | 12 | 0.0053 |
| 6 | Myc pathway | 368 | 136 | 65 | 18 | 48 | 18 | <0.00001 |
| 7 | p53 pathway | 1259 | 262 | 112 | 31 | 43 | 9 | <0.0001 |
| 8 | NF-kB pathway | 200 | 52 | 24 | 7 | 46 | 12 | 0.001 |

B. Genes changed concordantly between renal regeneration and RCC:

| | | | | | | | | |
|---|---|---|---|---|---|---|---|---|
| 1 | RCC | 984 | 361 | 278 | 77 | 77 | 28 | <0.00001A |
| 2 | VHL pathway | 282 | 104 | 59 | 16 | 57 | 21 | <0.00001 |
| 3 | Hypoxia pathway | 251 | 95 | 35 | 10 | 37 | 14 | <0.0001 |
| 4 | HRE target (HIF) | 39 | 17 | 4 | 1 | 24 | 10 | 0.2205 |
| 5 | IGF pathway | 139 | 37 | 9 | 3 | 24 | 7 | 0.4614 |
| 6 | Myc pathway | 368 | 136 | 55 | 15 | 40 | 15 | <0.00001 |
| 7 | p53 pathway | 1259 | 262 | 80 | 22 | 31 | 6 | 0.0043 |
| 8 | NF-kB pathway | 200 | 52 | 19 | 5 | 37 | 10 | 0.0027 |

C. Genes changed disconcordantly between renal regeneration and RCC:

| | | | | | | | | |
|---|---|---|---|---|---|---|---|---|
| 1 | RCC | 984 | 361 | 83 | 23 | 23 | 8 | <0.00001A |
| 2 | VHL pathway | 282 | 104 | 16 | 5 | 15 | 6 | <0.0001 |
| 3 | Hypoxia pathway | 251 | 95 | 16 | 4 | 17 | 6 | <0.0001 |
| 4 | HRE target (HIF) | 39 | 17 | 7 | 2 | 41 | 18 | <0.0001 |
| 5 | IGF pathway | 139 | 37 | 8 | 2 | 22 | 6 | <0.0001 |
| 6 | Myc pathway | 368 | 136 | 10 | 3 | 7 | 3 | 0.0551 |
| 7 | p53 pathway | 1259 | 262 | 32 | 9 | 12 | 3 | 0.0003 |
| 8 | NF-kB pathway | 200 | 52 | 5 | 2 | 10 | 3 | 0.3217 |

TABLE 6

| | | | | |
|---|---|---|---|---|
| RRR/ | RRR | Early | Late | Continues |
| RCC | pattern | I, E, early M | M, R | I, E, M, R |

| | | | |
|---|---|---|---|
| Concordance | regulation of | | |
| | translation | | |
| | physiological | | physiological processess |
| | processess | | |
| | biosynthesis | | biosynthesis |
| | cytosol | | cytosol |
| | | | structural molecule activity |
| | | | protein biosynthesis |
| | | | ribonucleoprotein protein |
| | | | ribosom |
| | | | structural constituent of ribosom |
| | | | macromolecule biosythesis |
| | | | cytosolic ribosome sensu Eukarya |
| | | | ribosome biogenesis and assembly |
| | | | ribosome biogenesis |
| | | | RNA binding |
| | | | cytoplasm organization and biogenesis |
| | | | cell organization and biogenesis |
| | | | small ribosomal subunit |
| | | | eukaryotic 43S pre-initiation complex |
| | | immunoglobulin | immunoglobulin binding |
| | | binding | |
| | | defense response | defense response |
| | | response to biotic | response to biotic stimulus |
| | | stimulus | |
| | | response to | response to external stimulus |
| | | external stimulus | |
| | | | protein-ER targeting |
| | | | posttranslational membrane targeting |
| | | | protein-membrane targeting |
| | | | ER organization and biogenesis |
| | DNA dependent DNA | | |
| | replication | | |
| | DNA replication | | |
| | intiation | | |
| | cell growth and/or | | |
| | maintenance | | |
| | oranic acid metabolism | | oranic acid metabolism |
| | carboxylic acid | | carboxylic acid metabolism |
| | metabolism | | |
| Discordance | growth factor binding | | |
| | | | organelle organization and biogenesis |
| | | extracellular space | |

TABLE 7
Gene Symbol

CRYM; CTGF; GPC3; CYR61; MYL6; TCF21; THBS1
FHL1; KDR; PKD1; RTN3; VEGF; GADD45G
AKAP2; MYL6; CORO1B
CD59; KIF21A; LPL; SCP2; ADD3; ARHE; MKNK2; NCOA4
AKAP2; APOE; NR2F6; CTGF; GC; CYR61; MYL6; SAR1; SLC1A1; CORO1B; SMC1L1; GPC3
ATP1B1; CAPNS1; CD59; CPT1A; FHL1; IGFBP1; IGFBP3; KIF21A; LPL; PKD1; RRM1; SCP2;
SLC16A7; SLC22A1; TOP3B; VEGF; ADD3; FRAP1; ARHE
NR2F6; SMC1L1
PKD1; RRM1; TOP3B; VEGF; FRAP1
FHL1; KDR; GADD45G
NR2F6; TCF21; ZNF144; SMC1L1
EIF4A2; TOP3B; NCOA4; PAPOLA; MKNK2
APOE^{HB}; IF; DCN; CTGF^{HB}; GC; GPC3; CYR61; MMP2; PLAT; SDC1; THBS1^{HB}; TACSTD2
BCKDHA; CD59; COX6C; IGFBP1; IGFBP3; KDR; Klk1; LPL^{HB}; MEP1A; ENPP2; RTN3;
VEGF^{HB}
CTGF; CYR61; THBS1
VEGF; KDR
SMC1L1
GADD45G; FRAP1^{REC}
IF; MMP2; PLAT
HK1; Klk1; LPL; AMACR; MEP1A; PGK1; SHMT1; ACOX1; CPT1A; SCP2
SAR1; SMC1L1^{ASE}
ATP1B1^{ASE}; EIF4A2^{ASE}; HARS; HK1; HSPH1; HSPD1; KDR; KIF21A; MKNK2; PCTK3; ARHE;
MTHFD1; MAT2A
BCKDHA; COX6C; CPT1A; HSPD1; AMACR; SCP2; SOD2
CTGF; THBS1
RTN3; GADD45G^{APO}
IF; FHIT; MMP2; PLAT; PPP2CB; PTPRO; SAR1; SMC1L1
ACOX1; ATP1B1; BCKDHA; CAPNS1; COX6C; CPT1A; EIF4A2; HARS; HK1; KDR; Klk1;
LPL; AMACR; MEP1A; MKNK2; PCTK3; ENPP2; PGK1; PAPOLA; PTPRB; RRM1; SCP2;
SHMT1; SOD2; TOP3B; FRAP1; ARHE; MTHFD1; MAT2A
IF; SMC1L1
HSPH1; HSPD1; SOD2; GADD45G; FRAP1
IF; RALBP1; TACSTD2
GJB2; HSPH1; HSPD1; PKD1; SOD2; GADD45G
AKAP2; NR2F6; CTGF; PTPRO; RALBP1; SAR1; TJP2; WSB1; IF; CYR61; THBS1; TACSTD2
KDR; PKD1; PTPRB; GADD45G; ARHE; IGFBP1; IGFBP3; VEGF; CEACAM1; GJB2
HARS; MTHFD1
IF; TACSTD2
GADD45G
ACOX1; BCKDHA; COX6C; RRM1; SOD2; MTHFD1
CTGF^{MIG}; FHIT; THBS1; MMP2; CYR61
RTN3^{MIG}; RRM1; CEACAM1; VEGF; ENPP2; GJB2; IGFBP3; CD59
CTGF; THBS1
CEACAM1; ARHE
CTGF; CYR61; Gpc3; Tacstd2
IGFBP1; IGFBP3; VEGF; Cox6c
FHIT; IF; MMP2; MT2A
CEACAM1; EIF4A2; FHL1; HSPH1; IGFBP3; MTHFD1; PCTK3; SHMT2; VEGF; CD59;
EGLN1; HSPD1
MMP2^{HIF}
CEACAM1; FHL1; IGFBP3^{HIF}; VEGF^{HIF}; CD59a^{HIF}; EGLN1^{HIF}; ATP1b1; SOD2; IGFBP1^{HIF};
GRSF1; HK1^{HIF}; ADD3; PGK1^{HIF}; PKD1; FRAP1
CTGF; THBS1
VEGF; GADD45G; GRSF1; PGK1; HSPH1; HSPD1; MAT2A; SHMT1
AKAP2; APOE; CYR61; FHIT; GPC3; MMP2; PLAT; PTPRO; RALBP1; SDC1; SLC1A1;
SMC111; THBS1; TJP2; ZNF144
ADD3; ATP1B1; CAPNS1; CD59; GJB2; HK1; HSPD1; HSPH1; IGFBP3; KDR; LPL; MTHFD1;
PKD1; RRM1; SOD2; TOP3b; VEGF
HSPD1; GFBP1; PGK1; SOD2; VEGF
FLAT
SOD2; IGFBP3; RRM1
FHIT; GPC3; TJP2
PKD1; RRM1
CYR61; GPC3; MMP2; NR2F6
EIF4A2; NCOA4
FHIT

TABLE 9

| | | | | | Concordant | |
|---|---|---|---|---|---|---|
| | | | | | (C) or | |
| | | Expression of | | | Disconcordant | |
| | | regeneration/normal: | | | (DC) with the | |
| | | Early(A)/Late(B)/ | RCC/ | | current renal | |
| | | both (*) Vs. Normal; | Normal | | regeneration | Hypoxia/ |
| Gene name | Symbol Human | (Up (+); Down (−)) | Kidney | RCC | dataset | Normoxia |
| S100 calcium binding protein A10 | S100A10 | (+) | | | | |
| (calpactin) | | | | | | |
| spermidine synthase | SRM | (+) | | | | |
| S100 calcium binding protein A6 | S100A6 | (+) | | | | |
| (calcyclin) | | | | | | |
| solute carrier family 26, member 4 | SLC26A4 | (−) | | | | |
| ajuba | JUB | (+) | | | | |
| keratin complex 1, acidic, gene 19 | KRT19 | (+) | (+) | RCC | C | (+) |
| RIKEN cD E130113K08 gene | T50835 | (+) | | | | |
| vascular cell adhesion molecule 1 | VCAM1 | (+) | (+) | RCC | C | |
| ectonucleoside triphosphate | ENTPD5 | (−) | | | | |
| diphosphohydrolase 5 | | | | | | |
| tuftelin 1 | TUFT1 | (+) | | | | |
| cell division cycle 42 homolog | CDC42 | (+) | (+) | RCC | C | (+) |
| (S. cerevisiae) | | | | | | |
| WNT1 inducible sigling pathway | WISP1 | (+) | | | | |
| protein 1 | | | | | | |
| cardiac responsive adriamycin protein | CARP | (+) | | | | |
| procollagen, type V, alpha 2 | COL5A2 | (+) | (+) | RCC | C | |
| heat shock 70 kDa protein 4 | HSPA4 | (+) | | | | |
| ATP-binding cassette, sub-family A | ABCA7 | (+) | | | | |
| (ABC1), member 7 | | | | | | |
| Mus musculus, Similar to | FLJ12618 | (−) | | | | |
| hypothetical protein FLJ12618, clone | | | | | | |
| MGC: 28775 IMAGE: 4487011, mR, | | | | | | |
| complete cds | | | | | | |
| DJ (Hsp40) homolog, subfamily B, | Djb12 | (−) | | | | |
| member 12 | | | | | | |
| ribosomal protein S19 | RPS19 | (+) | (+) | RCC | C | |
| mitochondrial ribosomal protein L39 | MRPL39 | (−) | | | | |
| tumor necrosis factor receptor | TNFRSF10B | (+) | | | | (+) |
| superfamily, member 10b | | | | | | |
| ATP synthase, H+ transporting | ATP5B | (−) | | | | |
| mitochondrial F1 complex, beta | | | | | | |
| subunit | | | | | | |
| golgi autoantigen, golgin subfamily a, 4 | GOLGA4 | (−) | | | | |
| cytochrome P450, 2d9 | CYP2D6 | (−) | | | | |
| tight junction protein 2 | TJP2 | (+) | (−) | RCC | DC | |
| serine protease inhibitor, Kunitz type 1 | SPINT1 | (+) | | | | |
| caspase 1 | CASP1 | (−) | (+)/(−) | RCC | conflict | |
| kynurenise (L-kynurenine hydrolase) | KYNU | (−) | | | | |
| histidyl tR synthetase | HARS | (−) | (+) | RCC | DC | |
| acetyl-Coenzyme A dehydrogese, | ACADM | (−) | | | | |
| medium chain | | | | | | |
| neutrophil cytosolic factor 2 | NCF2 | (+) | | | | |
| caspase 8 | CASP8 | (+) | | | | (+) |
| cell death-inducing D fragmentation | CIDEB | (−) | | | | (+) |
| factor, alpha subunit-like effector B | | | | | | |
| oncostatin receptor | OSMR | (+) | | | | |
| elafin-like protein I | SWAM1 | (−) | | | | |
| glutathione peroxidase 1 | GPX1 | (+) | (+) | RCC | C | |
| Rhesus blood group-associated C | RHCG | (−) | | | | |
| glycoprotein | | | | | | |
| GPI-anchored membrane protein 1 | M11S1 | (+) | (+) | RCC | C | |
| transcription elongation factor A | TCEA3 | (−) | | | | (+) |
| (SII), 3 | | | | | | |
| arachidote 12-lipoxygese, pseudogene 2 | ALOX12P2 | (−) | | | | |
| expressed in non-metastatic cells 2, | NME2 | (+) | (+) | RCC | C | |
| protein (NM23B) (nucleoside | | | | | | |
| diphosphate kise) | | | | | | |
| ribosomal protein S2 | RPS2 | (+) | (+) | RCC | C | |
| neural proliferation, differentiation | NPDC1 | (+) | (+) | RCC | C | |
| and control gene 1 | | | | | | |
| ribosomal protein L36 | RPL36 | (+) | (+) | RCC | C | |
| ribosomal protein S6 | RPS6 | (+) | | | | |
| hepatoma-derived growth factor | HDGF | (+) | | | | |
| DEAD/H (Asp-Glu-Ala-Asp/His) box | DDX50 | (+) | | | | |
| polypeptide 50/nucleolar protein | | | | | | |
| GU2 | | | | | | |
| SEC61, gamma subunit (S. cerevisiae) | SEC61G | (+) | (+)/(−) | RCC | conflict | |
| hypothetical protein, MNCb-5210 | COBRA1 | (+) | | | | |
| phosphofructokise, liver, B-type | PFKL | (−) | | | | (+) |
| D segment, Chr 12, ERATO Doi 604, | TSSC1 | (+) | | | | |
| expressed | | | | | | |
| carbonic anhydrase 5a, mitochondrial | CA5A | (−) | | | | |
| secreted and transmembrane 1 | SECTM1 | (−) | | | | |
| actin-like | ACTG1 | (+) | | | | |
| hyaluron mediated motility receptor | HMMR | (+) | | | | |
| (RHAMM) | | | | | | |
| complement component factor i | IF | (+) | (−) | RCC | DC | |
| carboxylesterase 3 | CES3 | (−) | | | | |
| ESTs, Weakly similar to T29029 | 4931439A04Rik | (+) | | | | |
| hypothetical protein F53G12.5 - | | | | | | |
| Caenorhabditis elegans (C. elegans) | | | | | | |
| RIKEN cD A330103N21 gene | A330103N21Rik | (−) | | | | |
| retinoblastoma binding protein 4 | RBBP4 | (+) | | | | |
| Mus musculus, Similar to 60S | | (−) | | | | |
| ribosomal protein L30 isolog, clone | | | | | | |
| MGC: 6735 IMAGE: 3590401, mR, | | | | | | |
| complete cds | | | | | | |
| cysteine rich protein 61 | CYR61 | (+) | (−) | RCC | DC | |
| growth arrest and D-damage- | GADD45A | (+) | | | | |
| inducible 45 alpha | | | | | | |
| centrin 3 | CETN3 | (+) | | | | |
| karyopherin (importin) alpha 2 | KPNA2 | (+) | (+) | RCC | C | |
| expressed sequence AW541137 | NUP107 | (+) | | | | |
| tumor necrosis factor receptor | TNFRSF1A | (+) | (+) | RCC | C | |
| superfamily, member 1a | | | | | | |
| alkaline phosphatase 2, liver | ALPL | (−) | (−) | RCC | C | |
| thioredoxin 1 | TXN | (+) | (−)/(+) | RCC | conflict | |
| ATPase, H+/K+ transporting, alpha | ATP4A | (−) | | | | |
| polypeptide | | | | | | |
| cytochrome P450, 2j5 | CYP2J2 | (−) | | | | |
| solute carrier family 22 (organic | Slc22al2 | (−) | | | | |
| cation transporter)-like 2 | | | | | | |
| eukaryotic translation initiation factor | EIF4A1 | (+) | (+) | RCC | C | |
| 4A1 | | | | | | |
| heparan sulfate 2-O-sulfotransferase 1 | HS2ST1 | (+) | | | | |
| microtubule-associated protein tau | MAPT | (−) | | | | |
| hydroxysteroid 17-beta dehydrogese 7 | HSD17B7 | (−) | | | | |
| dopa decarboxylase | DDC | (−) | (−) | RCC | C | |
| cytochrome c oxidase, subunit VIIa 1 | COX7A1 | (−) | | | | |
| ubiquitin specific protease 2 | USP2 | (−) | (−) | RCC | C | |
| fragile histidine triad gene | FHIT | (+) | (−) | RCC | DC | |
| ESTs, Weakly similar to ADT1 | | (−) | | | | |
| MOUSE ADP, ATP CARRIER | | | | | | |
| PROTEIN, HEART/SKELETAL | | | | | | |
| MUSCLE ISOFORM T1 | | | | | | |
| (M. musculus) | | | | | | |
| ganglioside-induced differentiation- | MRPS33 | (+) | | | | |
| associated-protein 3 | | | | | | |
| sideroflexin 1 | SFXN1 | (−) | | | | |
| SFFV proviral integration 1 | SPI1 | (+) | | | | |
| ribosomal protein L13a | RPL13A | (+) | (+) | RCC | C | |
| R polymerase I associated factor, 53 kD | PAF53 | (+) | | | | |
| Unknown | | (−) | | | | |
| ESTs | | (+) | | | | |
| expressed sequence AI450991 | KIAA0729 | (+) | | | | |
| importin 11 (RIKEN cD 2510001A17 | IPO11 | (+) | | | | |
| gene) | | | | | | |
| ESTs - pending | PCSK9 | (+) | | | | |
| SWI/SNF related, matrix associated, | SMARCA5 | (+) | (+) | RCC | C | |
| actin dependent regulator of | | | | | | |
| chromatin, subfamily a, member 5 | | | | | | |
| epidermal growth factor | EGF | (−) | (−) | RCC | C | |
| hypothetical protein, I54 | X61497 | (−) | | | | |
| mannose-6-phosphate receptor, cation | M6PR | (+) | | | | |
| dependent | | | | | | |
| urokise plasminogen activator | PLAUR | (+) | (+) | RCC | C | |
| receptor | | | | | | |
| ESTs | | (−) | | | | |
| chloride channel calcium activated 1 | CLCA1 | (+) | | | | |
| ornithine aminotransferase | OAT | (−) | | | | |
| Mus musculus, Similar to | C1QTNF5 | (+) | | | | |
| DKFZP586B0621 protein, clone | | | | | | |
| MGC: 38635 IMAGE: 5355789, mR, | | | | | | |
| complete cds | | | | | | |
| peroxisome proliferator activated | PPARA | (−) | | | | (−) |
| receptor alpha | | | | | | |
| RIKEN cD 4930552N12 gene | MCCC2 | (−) | | | | |
| RIKEN cD 2310009E04 gene | FLJ10986 | (−) | | | | (+) |
| ribosomal protein L41 | RPL41 | (+) | (+) | RCC | C | |
| RAB11a, member RAS oncogene | RAB11A | (+) | (+) | RCC | C | |
| family | | | | | | |
| apolipoprotein E | APOE | (+) | (−) | RCC | DC | |
| proteosome (prosome, macropain) | PSMB8 | (+) | (+) | RCC | C | |
| subunit, beta type 8 (large | | | | | | |
| multifunctiol protease 7) | | | | | | |
| osteomodulin | OMD | (−) | | | | |
| cytochrome c oxidase, subunit VIIIa | COX8 | (−) | | | | |
| RIKEN cD 2010012D11 gene | 2010012D11Rik | (−) | | | | |
| EGL nine homolog 1 (C. elegans) | EGLN1 | (−) | (+) | RCC | DC | (+) |
| DJ (Hsp40) homolog, subfamily C, | DNAJC5 | (+) | | | | (+) |
| member 5 | | | | | | |
| stearoyl-Coenzyme A desaturase 1 | SCD | (−) | | | | (+) |
| guanine nucleotide binding protein (G | GNG5 | (−) | | | | |
| protein), gamma 5 subunit | | | | | | |
| hydroxysteroid dehydrogese-1, | HSD3B2 | (−) | | | | |
| delta<5>-3-beta | | | | | | |
| bone morphogenetic protein receptor, | BMPR1A | (+) | | | | |
| type 1A | | | | | | |
| expressed sequence AI447451 | AI447451 | (+) | | | | |
| CEA-related cell adhesion molecule 1 | CEACAM1 | (−) | (+) | RCC | DC | (+) |
| lactate dehydrogese 1, A chain | LDHA | (+) | (+) | RCC | C | (+) |
| cold shock domain protein A | CSDA | (+) | (+) | RCC | C | |
| early development regulator 2 | EDR2 | (+) | | | | |
| (homolog of polyhomeotic 2) | | | | | | |
| a disintegrin-like and metalloprotease | ADAMTS1 | (+) | | | | |
| (reprolysin type) with | | | | | | |
| thrombospondin type 1 motif, 1 | | | | | | |
| ribosomal protein L27a | RPL27A | (+) | (+) | RCC | C | (+) |
| ribosomal protein, large P2 | RPLP2 | (+) | (+) | RCC | C | |
| solute carrier family 7 (cationic | SLC7A7 | (−) | (−) | RCC | C | |
| amino acid transporter, y+ system), | | | | | | |
| member 7 | | | | | | |
| acetyl-Coenzyme A acyltransferase 2 | ACAA2 | (−) | | | | |
| (mitochondrial 3-oxoacyl-Coenzyme | | | | | | |
| A thiolase) (D18Ertd240e) RIKEN | | | | | | |
| cD 0610011L04 gene | | | | | | |
| regulator of G-protein sigling 14 | RGS14 | (+) | | | | |
| thymosin, beta 4, X chromosome | TMSB4X | (+) | (+) | | C | (+) |
| metallothionein 2 | MT2A | (+) | (−) | RCC | DC | |
| serum amyloid A 3 | SAA3P | (+) | | | | |
| 2′-5′ oligoadenylate synthetase 1A | OAS1 | (+) | | | | |
| chemokine (C-C) receptor 5 | CCR5 | (+) | | | | |
| neurol guanine nucleotide exchange | NGEF | (−) | | | | |
| factor | | | | | | |
| f-box only protein 3 | FBXO3 | (−) | | | | |
| protein phosphatase 1, regulatory | PPP1R1A | (−) | | | | |
| (inhibitor) subunit 1A | | | | | | |
| phorbol-12-myristate-13-acetate- | PMAIP1 | (+) | | | | |
| induced protein 1 | | | | | | |
| NIMA (never in mitosis gene a)- | NEK6 | (+) | | | | (+) |
| related expressed kise 6 | | | | | | |
| transmembrane protein 8 (five | TMEM8 | (−) | | | | |
| membrane-spanning domains) | | | | | | |
| kallikrein 26 | Klk26 | (−) | | | | |
| protein tyrosine phosphatase, receptor | PTPRC | (+) | | | | |
| type, C | | | | | | |
| heat-responsive protein 12 | UK114 | (−) | (−) | RCC | C | |
| platelet derived growth factor, B | PDGFB | (+) | (+) | RCC | C | |
| polypeptide | | | | | | |
| RIKEN cD 1500026A19 gene | ALG5 | (+) | | | | |
| transforming growth factor, beta | TGFBI | (+) | (+) | RCC | C | (+) |
| induced, 68 kDa | | | | | | |
| baculoviral IAP repeat-containing 3 | BIRC3 | (+) | (+) | RCC | C | |
| small inducible cytokine A2 | SCYA2 | (+) | | | | |
| endothelin 1 | EDN1 | (+) | | | | (+) |
| dimethylarginine | DDAH2 | (+) | | | | |
| dimethylaminohydrolase 2 | | | | | | |
| phospholipid scramblase 1 | PLSCR1 | (+) | (+) | RCC | C | |
| translin | TSN | (+) | | | | |
| inhibitor of D binding 2 | ID2 | (+) | (+) | RCC | C | |
| reduced expression 3 | BEX1 | (−) | | | | |
| ribosomal protein S3 | RPS3 | (+) | (+) | RCC | C | (+) |
| cytochrome P450, 2a4 | CYP2A13 | (−) | | | | |
| MYB binding protein (P160) 1a | MYBBP1A | (+) | | | | |
| RIKEN cD 9530089B04 gene | 9530089B04Rik | (−) | | | | |
| malic enzyme, supertant | ME1 | (−) | | | | |
| ribosomal protein L44 | RPL36A | (+) | | | | |
| laminin B1 subunit 1 | LAMB1 | (+) | | | | |
| hemopoietic cell phosphatase | PTPN6 | (+) | (+) | RCC | C | |
| annexin A1 | ANXA1 | (+) | (+)/(???−) | RCC | conflict | |
| RIKEN cD 1110038J12 gene | | (−) | | | | |
| mini chromosome maintence | MCM4 | (+) | (+) | RCC | C | (+) |
| deficient 4 homolog (S. cerevisiae) | | | | | | |
| benzodiazepine receptor, peripheral | BZRP | (+) | | | | |
| solute carrier family 22 (organic | SLC22A1L | (−) | (−)/(+) | RCC | conflict | |
| cation transporter), member 1-like | | | | | | |
| karyopherin (importin) beta 3 | KPNB3 | (+) | | | | |
| lipoprotein lipase | LPL | (−) | (+) | RCC | DC | |
| ATP-binding cassette, sub-family D | ABCD3 | (−) | | | | |
| (ALD), member 3 | | | | | | |
| Mus musculus, Similar to RAS p21 | LOC218397 | (+) | | | | |
| protein activator, clone MGC: 7759 | | | | | | |
| IMAGE: 3498774, mR, complete cds | | | | | | |
| UDP-Gal:betaGlcc beta 1,3- | B3GALT3 | (−) | | | | |
| galactosyltransferase, polypeptide 3 | | | | | | |
| RIKEN cD 5031422I09 gene | PKP4 | (−) | | | | |
| Mus musculus, basic transcription | LOC218490 | (+) | | | | |
| factor 3, clone MGC: 6799 | | | | | | |
| IMAGE: 2648048, mR, complete cds | | | | | | |
| tumor-associated calcium sigl | TACSTD2 | (+) | (−) | RCC | DC | |
| transducer 2 | | | | | | |
| FK506 binding protein 5 (51 kDa) | FKBP5 | (−) | | | | |
| endoplasmic reticulum protein 29 | C12orf8 | (+) | | | | |
| plasminogen activator, tissue | PLAT | (+) | (−) | RCC | DC | |
| ribosomal protein S29 | RPS29 | (+) | | | | |
| cytochrome P450, family 4, | Cyp4v3 | (+) | | | | |
| subfamily v, polypeptide 3/ | | | | | | |
| expressed sequence AW111961 | | | | | | |
| CEA-related cell adhesion molecule 2 | Ceacam2 | (−) | | | | |
| downstream of tyrosine kise 1 | DOK1 | (+) | | | | |
| interleukin 11 receptor, alpha chain 1 | IL11RA | (−) | | | | |
| protein phosphatase 3, catalytic | PPP3CC | (−) | | | | |
| subunit, gamma isoform | | | | | | |
| granulin | GRN | (+) | (+) | RCC | C | |
| cathepsin Z | CTSZ | (+) | | | | |
| protease (prosome, macropain) 26S | PSMC1 | (+) | | | | |
| subunit, ATPase 1 | | | | | | |
| expressed sequence AW047581 | AW047581 | (+) | | | | |
| Mus musculus adult male kidney cD, | | (−) | | | | |
| RIKEN full-length enriched library, | | | | | | |
| clone: 0610012C11: homogentisate 1, | | | | | | |
| 2-dioxygese, full insert sequence | | | | | | |
| RIKEN cD 5730403B10 gene | C16orf5 | (−) | (+) | RCC | DC | |
| ESTs, Weakly similar to simple | | (+) | | | | |
| repeat sequence-containing transcript | | | | | | |
| (Mus musculus) (M. musculus) | | | | | | |
| T-cell specific GTPase | Tgtp | (+) | | | | |
| CD68 antigen | CD68 | (+) | (+) | RCC | C | |
| transmembrane 7 superfamily | TM7SF1 | (−) | | | | |
| member 1 | | | | | | |
| mitogen activated protein kise kise | MAP3K1 | (+) | | | | |
| kise 1 | | | | | | |
| retinoblastoma binding protein 7 | RBBP7 | (+) | (+) | RCC | C | |
| small inducible cytokine A7 | SCYA7 | (+) | | | | |
| cyclin E1 | CCNE1 | (+) | (+) | RCC | C | |
| coagulation factor II (thrombin) | F2RL1 | (+) | | | | |
| receptor-like 1 | | | | | | |
| annexin A5 | ANXA5 | (+) | | | | |
| Unknown | ITGA5 | (+) | | | | |
| beta-2 microglobulin | B2M | (+) | (+) | RCC | C | (+) |
| eukaryotic translation initiation factor | EIF4A2 | (−) | (+) | RCC | DC | |
| 4A2 | | | | | | |
| histocompatibility 2, class II, locus | HLA-DMA | (+) | | | | |
| DMa | | | | | | |
| ribosomal protein L35 | RPL35 | (+) | | | | |
| expressed sequence AW413625 | FLJ22794 | (+) | | | | |
| deltex 1 homolog (Drosophila) | DTX1 | (−) | (−) | RCC | C | |
| kinesin family member 1B (expressed | KIF1B | (+) | | | | |
| sequence AI448212) | | | | | | |
| transcription factor 21 | TCF21 | (+) | (−) | RCC | DC | |
| nuclear receptor subfamily 2, group | NR2F2 | (+) | (+) | RCC | C | |
| F, member 2 | | | | | | |
| R polymerase II 1 | POLR2A | (−) | | | | |
| actin, alpha 2, smooth muscle, aorta | ACTA2 | (+) | | | | |
| neural precursor cell expressed, | NEDD4 | (−) | | | | |
| developmentally down-regulated gene | | | | | | |
| 4a | | | | | | |
| actin, gamma 2, smooth muscle, | ACTG2 | (+) | | | | |
| enteric | | | | | | |
| mini chromosome maintence | MCM2 | (+) | (+) | RCC | C | |
| deficient 2 (S. cerevisiae) | | | | | | |
| integrin-associated protein | CD47 | (+) | (+)/?) | RCC | conflict | |
| creatine kise, brain | CKB | (−) | | | | (+) |
| 3-phosphoglycerate dehydrogese | PHGDH | (+) | (−)/(+) | RCC | conflict | |
| ESTs, Weakly similar to 2022314A | | (+) | | | | |
| granule cell marker protein | | | | | | |
| (M. musculus) | | | | | | |
| TAF9 R polymerase II, TATA box | TAF9 | (+) | | | | |
| binding protein (TBP)-associated | | | | | | |
| factor, 32 kDa | | | | | | |
| Ral-interacting protein 1 | RALBP1 | (+) | (−) | RCC | DC | |
| tubulin, beta 5 | TUBB | (+) | (+) | RCC | C | |
| speckle-type POZ protein | SPOP | (−) | | | | |
| amelogenin | AMELX | (+) | | | | |
| tropomyosin 3, gamma | TPM3 | (+) | | | | |
| solute carrier family 22 (organic | SLC22A2 | (−) | | | | |
| cation transporter), member 2 | | | | | | |
| CD48 antigen | CD48 | (+) | | | | |
| RIKEN cD 1200014I03 gene | F13A1 | (+) | | | | |
| avian reticuloendotheliosis viral (v- | RELB | (+) | | | | |
| rel) oncogene related B | | | | | | |
| growth factor receptor bound protein 7 | GRB7 | (−) | (−) | RCC | C | |
| histocampatibility 2, class II antigen | HLA-DQA1 | (+) | | | | |
| A, alpha | | | | | | |
| proteasome (prosome, macropain) | PSMD10 | (+) | | | | |
| 26S subunit, non-ATPase, 10 | | | | | | |
| hematological and neurological | HN1 | (+) | (+) | RCC | C | |
| expressed sequence 1 | | | | | | |
| heat shock protein 1 (chaperonin)/ | HSPD1 | (−) | (+) | RCC | DC | |
| heat shock protein, 60 kDa | | | | | | |
| sterol carrier protein 2, liver | SCP2 | (−) | (+) | RCC | DC | |
| RIKEN cD 1110054A24 gene | 1110054A24Rik | (+) | | | | |
| crystallin, alpha B | CRYAB | (+) | (+) | RCC | C | |
| RIKEN cD 2410026K10 gene | CD99 | (+) | | | | (+) |
| adenine phosphoribosyl transferase | APRT | (+) | | | | |
| lectin, galactose binding, soluble 4 | LGALS4 | (−) | | | | |
| Arpc2 | ARPC2 | (+) | | | | |
| RIKEN cD 2600015J22 gene | | (+) | | | | |
| heme oxygese (decycling) 1 | HMOX1 | (+) | | | | (+) |
| ubiquitin-conjugating enzyme E2D 2 | UBE2D2 | (+) | | | | |
| ubiquitin-conjugating enzyme E2H | UBE2H | (+) | (+) | RCC | C | (+) |
| glucose-6-phosphatase, catalytic | G6PC | (−) | | | | |
| Rap1, GTPase-activating protein 1 | RAP1GA1 | (−) | (−) | RCC | C | |
| lectin, galactose binding, soluble 9 | LGALS9 | (+) | (+)/.(− | RCC | conflict | |
| | | | ???) | | | |
| dihydropyrimidise-like 3 | DPYSL3 | (+) | (+) | RCC | C | |
| bisphosphate 3′-nucleotidase 1 | BPNT1 | (−) | | | | |
| connective tissue growth factor | CTGF | (+) | (−) | RCC | DC | |
| procollagen, type IV, alpha 2 | COL4A2 | (+) | (+) | RCC | C | |
| RIKEN cD 0610007L01 gene | FLJ10099 | (+) | | | | |
| cytidine 5′-triphosphate synthase | CTPS | (+) | | | | |
| RIKEN cD 4430402G14 gene | H3f3b | (+) | | | | |
| mutS homolog 6 (E. coli) | MSH6 | (+) | | | | |
| CDC16 (cell division cycle 16 | CDC16 | (+) | (+) | RCC | C | |
| homolog (S. cerevisiae) | | | | | | |
| RIKEN cD 5730534O06 gene | KIAA0164 | (−) | | | | |
| RIKEN cD 2610524G07 gene | | (−) | | | | |
| proteasome (prosome, macropain) | PSMA2 | (+) | | | | |
| subunit, alpha type 2 | | | | | | |
| solute carrier family 3, member 1 | SLC3A1 | (−) | (−) | RCC | C | |
| RIKEN cD 2310051E17 gene | 2310051E17Rik | (−) | | | | |
| lyric (D8Bwg1112e) D segment, Chr | LYRIC | (+) | | | | |
| 8, Brigham & Women's Genetics | | | | | | |
| 1112 expressed | | | | | | |
| tescin XB | TNXB | (−) | | | | |
| Yamaguchi sarcoma viral (v-yes-1) | LYN | (+) | (+) | RCC | C | |
| oncogene homolog | | | | | | |
| cytochrome P450, subfamily IV B, | CYP4B1 | (−) | | | | |
| polypeptide 1 | | | | | | |
| microtubule-associated protein, | MAPRE1 | (+) | | | | |
| RP/EB family, member 1 | | | | | | |
| heat shock protein, 86 kDa 1 | HSPCA | (+) | (?) | RCC | conflict | |
| pyruvate decarboxylase | PC | (−) | | | | |
| oxysterol binding protein-like 1A | OSBPL1A | (−) | | | | |
| carnitine palmitoyltransferase 1, liver | CPT1A | (−) | (+) | RCC | DC | |
| UDP-N-acetyl-alpha-D- | GALGT | (+) | | | | |
| galactosamine:(N-acetylneuraminyl)- | | | | | | |
| galactosylglucosylceramide-beta-1,4- | | | | | | |
| N-acetylgalactosaminyltransferase | | | | | | |
| zinc finger protein 36, C3H type-like 1 | ZFP36L1 | (+) | (+) | RCC | C | (+) |
| acyl-Coenzyme A dehydrogese, very | ACADVL | (−) | | | | |
| long chain | | | | | | |
| aminoadipate-semialdehyde synthase/ | AASS | (−) | | | | |
| (Lorsdh) lysine oxoglutarate | | | | | | |
| reductase, saccharopine dehydrogese | | | | | | |
| RIKEN cD 1110014C03 gene | TMP21 | (+) | | | | |
| FXYD domain-containing ion | FXYD5 | (+) | | | | |
| transport regulator 5 | | | | | | |
| expressed sequence AI316828 | FLJ20618 | (+) | | | | |
| phosphoglycerate kise 1 | PGK1 | (−) | (+) | RCC | DC | (+) |
| Unknown | | (+) | | | | |
| RIKEN cD 1700008H23 gene | 1700008H23Rik | (−) | | | | |
| RIKEN cD 2810047L02 gene | RAMP | (+) | | | | |
| mini chromosome maintence | MCM7 | (+) | (+) | RCC | C | |
| deficient 7 (S. cerevisiae) | | | | | | |
| RIKEN cD 2410174K12 gene | SUGT1 | (+) | | | | |
| polypyrimidine tract binding protein 1 | PTBP1 | (+) | (+) | RCC | C | (+) |
| complement component 3 | C3 | (+) | | | | |
| succite-Coenzyme A ligase, ADP- | SUCLA2 | (−) | | | | |
| forming, beta subunit | | | | | | |
| thioredoxin-like (32 kD) | TXNL | (+) | | | | |
| methionine aminopeptidase 2 | METAP2 | (+) | | | | |
| hepsin | HPN | (−) | (−) | RCC | C | |
| T-cell, immune regulator 1 | TCIRG1 | (+) | | | | |
| prothymosin alpha | PTMA | (+) | (+) | RCC | C | |
| RIKEN cD 0610006F02 gene | DKFZP566H073 | (−) | | | | |
| solute carrier family 13 | SLC13A1 | (+) | | | | |
| (sodium/sulphate symporters), | | | | | | |
| member 1 | | | | | | |
| Mus musculus, clone | | (+) | | | | |
| IMAGE: 3494258, mR, partial cds | | | | | | |
| matrix gamma-carboxyglutamate | MGP | (+) | | | | |
| (gla) protein | | | | | | |
| leucocyte specific transcript 1 | LY117 | (+) | (+) | RCC | C | |
| Mus musculus, Similar to | FLJ21634 | (−) | | | | |
| hypothetical protein FLJ21634, clone | | | | | | |
| MGC: 19374 IMAGE: 2631696, mR, | | | | | | |
| complete cds | | | | | | |
| complement factor H related protein | HF1 | (+) | | | | |
| 3A4/5G4 | | | | | | |
| RIKEN cD 2610200M23 gene | SSBP3 | (+) | (+) | RCC | C | |
| (Prlr-rs1) prolactin receptor related | PRLR | (−) | | | | |
| sequence 1 | | | | | | |
| sigl transducer and activator of | STAT3 | (+) | (+) | RCC | C | |
| transcription 3 | | | | | | |
| peptidylprolyl isomerase | PPIL1 | (+) | (+) | RCC | C | |
| (cyclophilin)-like 1 | | | | | | |
| histocompatibility 2, L region | H2-L | (+) | | | | |
| eukaryotic translation initiation factor | eIF2a | (+) | | | | |
| 2A | | | | | | |
| serine/arginine repetitive matrix 1 | RAD23B | (+) | | | | |
| solute carrier family 31, member 1 | SLC31A1 | (−) | | | | |
| clusterin | CLU | (+) | (?) | RCC | conflict | |
| yolk sac gene 2 | DKFZp761A051.1 | (−) | | | | |
| tubulin alpha 1 | TUBA1 | (+) | | | | |
| guanine nucleotide binding protein, | GNAI2 | (+) | (+) | RCC | C | |
| alpha inhibiting 2 | | | | | | |
| Unknown | | (+) | | | | |
| selenium binding protein 2 | SELENBP1 | (−) | (+) | RCC | C | |
| group specific component | GC | (+) | (−) | RCC | DC | |
| hexokise 1 | HK1 | (−) | (+) | RCC | DC | (+) |
| eukaryotic translation initiation factor | EIF5A | (+) | | | | |
| 5A | | | | | | |
| glycoprotein 49 A | Gp49a | (+) | | | | |
| CDK2 (cyclin-dependent kise 2)- | CDK2AP1 | (+) | | | | |
| asscoaited protein 1 | | | | | | |
| core promoter element binding | COPEB | (+) | (+) | RCC | C | |
| protein | | | | | | |
| B-cell leukemia/lymphoma 2 related | BCL2A1 | (+) | | | | |
| protein A1b | | | | | | |
| RIKEN cD 5430416A05 gene | AD034 | (+) | | | | |
| protein phosphatase 1, catalytic | PPP1CA | (+) | | | | |
| subunit, alpha isoform | | | | | | |
| calreticulin | CALR | (+) | (−)/(+) | RCC | conflict | |
| RAS-related C3 botulinum substrate 2 | RAC2 | (+) | | | | |
| glutathione S-transferase, alpha 2 | GSTA2 | (−) | (+)/(−) | RCC | conflict | |
| (Yc2) | | | | | | |
| tubulin alpha 2 | TUBA2 | (+) | | | | |
| lysosomal-associated protein | LAPTM4B | (+) | | | | |
| transmembrane 4B | | | | | | |
| Mitogen activated protein kinase 1; | MAPK1 | (−) | | | | (+) but |
| RIKEN cD 9030612K14 gene | | | | | | blocked |
| | | | | | | HIF-1 |
| | | | | | | activation |
| | | | | | | by hypoxia |
| X (ictive)-specific transcript, | TSIX | (+) | | | | |
| antisense | | | | | | |
| expressed sequence C80913 | C80913 | (+) | | | | |
| Kruppel-like factor 9 | BTEB1 | (−) | | | | |
| arachidote 5-lipoxygese activating | ALOX5AP | (+) | (+) | RCC | C | |
| protein | | | | | | |
| decorin | DCN | (+) | (−) | RCC | DC | |
| Mus musculus, Similar to Protein P3, | DXS253E | (+) | | | | |
| clone MGC: 38638 IMAGE: 5355849, | | | | | | |
| mR, complete cds | | | | | | |
| matrix metalloproteise 14 | MMP14 | (+) | (+) | RCC | C | |
| (membrane-inserted) | | | | | | |
| expressed sequence AA672638 | AA672638 | (−) | | | | |
| RIKEN cD A230106A15 gene | A230106A15Rik | (−) | | | | |
| expressed sequence AA589392 | AA589392 | (+) | | | | |
| expressed sequence AI838057 | AI838057 | (−) | | | | |
| transgelin | TAGLN | (+) | | | | |
| LIM and SH3 protein 1 | LASP1 | (+) | | | | |
| expressed sequence AI843960 | RBPSUH | (+) | | | | |
| Mus musculus, clone | TOR2A | (+) | | | | |
| IMAGE: 4952483, mR, partial cds | | | | | | |
| RIKEN cD 2410129E14 gene | | (+) | | | | |
| ((AW146109) expressed sequence | CD44 | (+) | (+) | | C | |
| AW146109) | | | | | | |
| D-amino acid oxidase | DAO | (−) | | | | |
| expressed sequence AI593524 | DKFZp586A011.1 | (−) | | | | |
| expressed sequence AI607846 | AIF1 | (+) | | | | |
| RIKEN cD 1190006C12 gene | SEC61B | (+) | | | | |
| mannose receptor, C type 1 | MRC1 | (+) | | | | |
| phospholipase A2, group IB, pancreas | PLA2G1B | (+) | | | | |
| adenylate cyclase 4 | ADCY4 | (−) | | | | |
| aquaporin 2 | AQP2 | (−) | | | | |
| expressed sequence AI182284 | AI182284 | (−) | | | | |
| baculoviral IAP repeat-containing 2 | BIRC2 | (+) | (+) | RCC | C | |
| malonyl-CoA decarboxylase | MLYCD | (−) | | | | |
| Muf1 protein (D630045E04Rik) Mus | MUF1 | (+) | | | | |
| musculus, clone IMAGE: 3491421, | | | | | | |
| mR, partial cds | | | | | | |
| RIKEN cD 2610007A16 gene | SEC13L | (−) | | | | |
| selenophosphate synthetase 2 | SPS2 | (−) | (−) | RCC | C | |
| apurinic/apyrimidinic endonuclease | APEX1 | (+) | | | | (+) |
| MAD homolog 5 (Drosophila)/ | MADH5 | (+) | (+) | RCC | C | |
| expressed sequence AI451355 | | | | | | |
| dipeptidase 1 (rel) | DPEP1 | (−) | (−) | RCC | C | |
| expressed sequence AI132321 | AI132321 | (+) | | | | |
| expressed sequence AI159688 | AI159688 | (−) | | | | |
| gamma-glutamyl hydrolase | GGH | (+) | (+)/(−) | RCC | conflict | |
| Mus musculus, Similar to | FLJ20234 | (+) | | | | |
| hypothetical protein FLJ20234, clone | | | | | | |
| MGC: 37525 IMAGE: 4986113, mR, | | | | | | |
| complete cds | | | | | | |
| expressed sequence AL022757 | 5730453I16Rik | (+) | | | | |
| Mus musculus, clone MGC: 38798 | MGC38798 | (−) | | | | |
| IMAGE: 5359803, mR, complete cds | | | | | | |
| Mus musculus, Similar to cortactin | EMS1 | (+) | | | | |
| isoform B, clone MGC: 18474 | | | | | | |
| IMAGE: 3981559, mR, complete cds | | | | | | |
| Mus musculus, clone MGC: 18985 | FLJ20303 | (+) | (+) | RCC | C | |
| IMAGE: 4011674, mR, complete cds | | | | | | |
| Mus musculus, Similar to | FLJ10520 | (−) | | | | |
| hypothetical protein FLJ10520, clone | | | | | | |
| MGC: 27888 IMAGE: 3497792, mR, | | | | | | |
| complete cds | | | | | | |
| pyridoxal (pyridoxine, vitamin B6) | PDXK | (+) | | | | |
| kise | | | | | | |
| Mus musculus mR for 67 kDa | EIF3S6IP | (+) | | | | |
| polymerase-associated factor PAF67 | | | | | | |
| (paf67 gene) | | | | | | |
| cytidine 5′-triphosphate synthase 2 | CTPS2 | (+) | | | | |
| Unknown | | (+) | | | | |
| epithelial membrane protein 3 | EMP3 | (+) | (+) | RCC | C | |
| ceroid-lipofuscinosis, neurol 2 | CLN2 | (−) | | | | |
| solute carrier family 22 (organic | SLC22A8 | (−) | (−) | RCC | C | |
| anion transporter), member 8/(Roct) | | | | | | |
| reduced in osteosclerosis transporter | | | | | | |
| erythrocyte protein band 4.1-like 1 | EPB41L1 | (−) | | | | |
| low density lipoprotein receptor- | LRP6 | (−) | | | | |
| related protein 6 | | | | | | |
| trinucleotide repeat containing 11 | TNRC11 | (+) | | | | |
| (THR-associated protein, 230 kDa | | | | | | |
| subunit) | | | | | | |
| src homology 2 domain-containing | SHD | (−) | | | | (+) |
| transforming protein D | | | | | | |
| ribosomal protein S6 kise, 90 kD, | RPS6KA4 | (+) | | | | |
| polypeptide 4 | | | | | | |
| topoisomerase (D) III beta | TOP3B | (−) | (+) | RCC | DC | |
| G1 to phase transition 1 | GSPT1 | (+) | | | | |
| transforming growth factor beta 1 | TSC22 | (+) | (+) | RCC | C | |
| induced transcript 4 | | | | | | |
| mitsugumin 29 | Mg29 | (−) | | | | |
| FK506 binding protein 9 | FKBP9 | (+) | | | | |
| regulator of G-protein sigling 19 | RGS19IP1 | (+) | | | | |
| interacting protein 1 | | | | | | |
| transcobalamin 2 | TCN2 | (−) | (−) | RCC | C | |
| thioesterase, adipose associated | THEA | (−) | | | | |
| lysyl oxidase-like | LOXL1 | (+) | | | | |
| nuclease sensitive element binding | NSEP1 | (+) | (+) | RCC | C | |
| protein 1 | | | | | | |
| transthyretin | TTR | (−) | | | | |
| RIKEN cD 5630401J11 gene | 5630401J11Rik | (+) | | | | |
| LPS-induced TNF-alpha factor | LITAF | (+) | | | | |
| FK506 binding protein 12-rapamycin | FRAP1 | (−) | (+) | RCC | DC | Frap1 |
| associated protein 1 | | | | | | amplified |
| | | | | | | HIF |
| | | | | | | signaling |
| interferon activated gene 204 | Ifi204 | (+) | | | | |
| insulin-like growth factor binding | IGFBP1 | (−) | (+) | RCC | DC | (+) |
| protein 1 | | | | | | |
| myeloid differentiation primary | MYD88 | (+) | | | | |
| response gene 88 | | | | | | |
| Mus musculus, similar to | MGC37309 | (+) | | | | |
| heterogeneous nuclear | | | | | | |
| ribonucleoprotein A3 (H. sapiens), | | | | | | |
| clone MGC: 37309 IMAGE: 4975085, | | | | | | |
| mR, complete cds | | | | | | |
| elastase 1, pancreatic | ELA1 | (−) | | | | |
| craniofacial development protein 1 | CFDP1 | (+) | | | | |
| folate receptor 1 (adult) | FOLR1 | (−) | (−)/(+) | RCC | conflict | |
| proteaseome (prosome, macropain) | PSME3 | (−) | | | | |
| 28 subunit, 3 | | | | | | |
| TAF10 R polymerase II, TATA box | TAF10 | (+) | | | | |
| binding protein (TBP)-associated | | | | | | |
| factor, 30 kDa | | | | | | |
| E-vasodilator stimulated | EVL | (+) | (+) | RCC | C | |
| phosphoprotein | | | | | | |
| EST AI181838 | MGC2555 | (−) | | | | |
| cathepsin D | CTSD | (+) | (+) | RCC | C | (+) |
| opioid growth factor receptor | OGFR | (+) | | | | |
| chloride channel, nucleotide- | CLNS1A | (+) | | | | |
| sensitive, 1A | | | | | | |
| Mus musculus, Similar to retinol | RODH-4 | (−) | | | | |
| dehydrogese type 6, clone | | | | | | |
| MGC: 25965 IMAGE: 4239862, mR, | | | | | | |
| complete cds | | | | | | |
| actin, alpha 1, skeletal muscle | ACTA1 | (+) | | | | |
| cytochrome c oxidase, subunit VIIa 3 | COX7A3 | (−) | | | | |
| expressed sequence C85457 | C85457 | (−) | | | | |
| H2B histone family, member S | H2BFS | (−) | | | | |
| Mus musculus, similar to quinone | VAT1 | (−) | | | | |
| reductase-like protein, clone | | | | | | |
| IMAGE: 4972406, mR, partial cds | | | | | | |
| ESTs, Weakly similar to S26689 | | (−) | | | | |
| hypothetical protein hc1 - mouse | | | | | | |
| (M. musculus) | | | | | | |
| reticulon 3 | RTN3 | (−) | (+) | RCC | DC | |
| striatin, calmodulin binding protein 4/ | STRN4 | (+) | | | | |
| expressed sequence C80611 | | | | | | |
| ESTs | | (−) | | | | |
| Mus musculus, similar to R29893-1, | | (−) | | | | |
| clone MGC: 37808 IMAGE: 5098192, | | | | | | |
| mR, complete cds | | | | | | |
| RIKEN cD 3110001N18 gene | RPL22 | (+) | (+) | RCC | C | (+) |
| proteasome (prosome, macropain) | PSMA7 | (+) | (+) | RCC | C | |
| subunit, alpha type 7 | | | | | | |
| cytochrome P450, 2el, ethanol | CYP2E1 | (−) | | | | |
| inducible | | | | | | |
| small nuclear ribonucleoprotein | SNRPG | (+) | | | | |
| polypeptide G | | | | | | |
| calponin 2 | CNN2 | (+) | | | | |
| RIKEN cD 1200014D15 gene | DMGDH | (−) | | | | |
| ESTs, Weakly similar to | | (−) | | | | |
| TYROSINE-PROTEIN KISE JAK3 | | | | | | |
| (M. musculus) | | | | | | |
| lymphocyte specific 1 | LSP1 | (+) | (+) | RCC | C | |
| RIKEN cD 4930542G03 gene | 4930542G03Rik | (+) | | | | |
| ESTs | | (+) | | | | |
| splicing factor, arginine/serine-rich 2 | SFRS2 | (+) | (+) | RCC | C | |
| (SC-35) | | | | | | |
| peroxisomal membrane protein 2, 22 kDa | PXMP2 | (−) | (+)/(−) | RCC | conflict | |
| ESTs, Moderately similar to S12207 | | (−) | | | | |
| hypothetical protein (M. musculus) | | | | | | |
| Unknown | | (−) | | | | |
| CD2-associated protein | CD2AP | (+) | (+) | RCC | C | |
| expressed sequence AI182282 | SLC9A8 | (−) | | | | |
| vascular endothelial zinc finger 1; | Vezf1 | (−) | | | | |
| expressed sequence AI848691 | | | | | | |
| RIKEN cD 1810038D15 gene | DKFZP566E144 | (+) | | | | |
| ESTs | | (−) | | | | |
| solute carrier family 34 (sodium | SLC34A1 | (−) | | | | |
| phosphate), member 1 | | | | | | |
| phosphoglycerate mutase 2 | PGAM2 | (−) | | | | |
| metallothionein 1 | MT1A | (+) | | | | |
| Mus musculus, clone | APEH | (−) | (−) | RCC | C | |
| IMAGE: 4974221, mR, partial cds | | | | | | |
| histone 2, H2aa1/(Hist2) histone | HIST2H2AA | (−) | | | | |
| gene complex 2 | | | | | | |
| epidermal growth factor-containing | EFEMP1 | (+) | | | | |
| fibulin-like extracellular matrix | | | | | | |
| protein 1 | | | | | | |
| betaine-homocysteine | BHMT | (−) | (−) | RCC | C | |
| methyltransferase | | | | | | |
| junction plakoglobin | JUP | (−) | (−) | RCC | C | |
| hepatic nuclear factor 4 | HNF4A | (−) | | | | Hnf4 |
| | | | | | | interact |
| | | | | | | with |
| | | | | | | HIF1a & |
| | | | | | | ARNT |
| expressed sequence AI194696 | HFL1 | (+) | | | | |
| Mus musculus, clone MGC: 7898 | | (−) | | | | |
| IMAGE: 3582717, mR, complete cds | | | | | | |
| RIKEN cD 2700038K18 gene | | (+) | | | | |
| Fc receptor, IgG, low affinity III | FCGR3A | (+) | (+) | RCC | C | |
| succite dehydrogese complex, subunit | SDHA | (−) | | | | |
| A, flavoprotein (Fp) | | | | | | |
| interleukin 1 beta | IL1B | (+) | (?) | RCC | conflict | |
| RIKEN cD 2700027J02 gene | SPF45 | (+) | | | | |
| selectin, platelet (p-selectin) ligand | SELPLG | (+) | (+) | RCC | C | |
| RIKEN cD 1200009B18 gene | LOC51290 | (+) | | | | |
| proteoglycan, secretory granule | PRG1 | (+) | (+) | RCC | C | |
| transformation related protein 53 | TP53 | (+) | (+)/(−??) | RCC | conflict | (+) |
| carboxypeptidase X 1 (M14 family)/ | CPXM | (+) | | | | |
| metallocarboxypeptidase 1 | | | | | | |
| SH3 domain binding glutamic acid- | SH3BGRL3 | (+) | | | | (+) |
| rich protein-like 3 | | | | | | |
| insulin-like growth factor binding | IGFBP4 | (−) | | | | |
| protein 4 | | | | | | |
| exportin 1, CRM1 homolog (yeast) | XPO1 | (+) | (+) | RCC | C | |
| Mus musculus, clone MGC: 38363 | TM4SF3 | (+) | (−) | RCC | DC | |
| IMAGE: 5344986, mR, complete cds | | | | | | |
| RIKEN cD 2310046G15 gene | SPUVE | (+) | (+) | RCC | C | |
| ribosomal protein L29 | RPL29 | (+) | (+) | RCC | C | (+) |
| E26 avian leukemia oncogene 2,3′ | ETS2 | (+) | | | | |
| domain | | | | | | |
| Mus musculus, Similar to | FLJ13213 | (+) | | | | |
| hypothetical protein FLJ13213, clone | | | | | | |
| MGC: 28555 IMAGE: 4206928, mR, | | | | | | |
| complete cds | | | | | | |
| eukaryotic translation initiation factor 3 | EIF3S10 | (+) | | | | |
| Mus musculus, Similar to | DKFZp566A1524 | (+) | | | | |
| hypothetical protein | | | | | | |
| DKFZp566A1524, clone MGC: 18989 | | | | | | |
| IMAGE: 4012217, mR, complete cds | | | | | | |
| RIKEN cD 1300013G12 gene | 1300013G12Rik | (+) | | | | (+) |
| chloride intracellular channel 4 | CLIC4 | (+) | | | | |
| (mitochondrial) | | | | | | |
| activator of S phase kise | ASK | (+) | | | | |
| ketohexokise | KHK | (−) | (−) | RCC | C | |
| expressed sequence AI265322 | AI265322 | (−) | | | | |
| glypican 3 | GPC3 | (+) | (−) | RCC | DC | |
| EGF-like module containing, mucin- | EMR1 | (+) | | | | |
| like, hormone receptor-like sequence 1 | | | | | | |
| diaphorase 1 (DH) | DIA1 | (+) | | | | |
| histocompatibility 2, class II antigen | H2-Eb1 | (+) | | | | |
| E beta | | | | | | |
| melanoma antigen, family D, 2 | MAGED2 | (+) | | | | |
| serine/threonine kise receptor | UNRIP | (+) | | | | |
| associated protein | | | | | | |
| annexin A6 | ANXA6 | (+) | | | | |
| procollagen, type I, alpha 1 | COL1A1 | (+) | (+)/(−?) | RCC | conflict | |
| Mus musculus, Similar to transgelin | TAGLN2 | (+) | (+) | RCC | C | |
| 2, clone MGC: 6300 | | | | | | |
| IMAGE: 2654381, mR, complete cds | | | | | | |
| RIKEN cD 2810409H07 gene | PTD004 | (+) | | | | |
| transformed mouse 3T3 cell double | MDM2 | (+) | (+) | RCC | C | |
| minute 2 | | | | | | |
| Fc receptor, IgE, high affinity I, | FCER1G | (+) | (+) | RCC | C | |
| gamma polypeptide | | | | | | |
| selenoprotein P, plasma, 1 | SEPP1 | (−) | (−) | RCC | C | |
| serine (or cysteine) proteise inhibitor, | SERPINH1 | (+) | | | | |
| clade H (heat shock protein 47), | | | | | | |
| member 1 | | | | | | |
| small inducible cytokine A9 | CCL9 | (+) | | | | |
| phospholipase A2, activating protein | PLAA | (+) | | | | |
| FXYD domain-containing ion | FXYD2 | (−) | (−) | RCC | C | |
| transport regulator 2 | | | | | | |
| cordon-bleu; ESTs, Moderately | COBL | (+) | | | | |
| similar to T00381 KIAA0633 protein | | | | | | |
| (H. sapiens) | | | | | | |
| expressed sequence AW488255 | EFNB1 | (−) | | | | |
| Mus musculus, clone | | (+) | | | | |
| IMAGE: 4486265, mR, partial cds | | | | | | |
| protein kise C, delta | PRKCD | (+) | (+) | RCC | C | |
| RIKEN cD 2310067B10 gene | KIAA0195 | (−) | | | | |
| RIKEN cD 9130011J04 gene | 9130011J04Rik | (+) | | | | |
| RIKEN cD 3230402E02 gene | FLJ10983 | (+) | (+) | RCC | C | |
| macrophage migration inhibitory | MIF | (−) | | | | |
| factor | | | | | | |
| RIKEN cD 0610041E09 gene | AD-020 | (+) | | | | |
| glutamine synthetase | GLUL | (−) | | | | |
| prohibitin | PHB | (−) | | | | |
| RIKEN cD 6330583M11 gene | DKFZP434P106 | (+) | (+) | RCC | C | |
| tumor protein p53 binding protein, 2/ | TP53BP2 | (−) | | | | |
| expressed sequence AI746547 | | | | | | |
| expressed sequence AI315037 | AI315037 | (−) | | | | |
| nestin - pendin | NES | (+) | | | | |
| nuclear receptor subfamily 2, group | NR2F6 | (+) | (−) | RCC | DC | |
| F, member 6 | | | | | | |
| Mus musculus, clone | YUP8H12R.13 | (+) | | | | |
| IMAGE: 3994696, mR, partial cds | | | | | | |
| golgi reassembly stacking protein 2 | GORASP2 | (+) | (+) | RCC | C | |
| low density lipoprotein receptor- | LRP2 | (−) | (−) | RCC | C | |
| related protein 2 | | | | | | |
| ESTs, Weakly similar to YAE6- | | (−) | | | | |
| YEAST HYPOTHETICAL 13.4 KD | | | | | | |
| PROTEIN IN ACS1-GCV3 | | | | | | |
| INTERGENIC REGION | | | | | | |
| (S. cerevisiae) | | | | | | |
| Cbp/p300-interacting transactivator | CITED1 | (−) | | | | |
| with Glu/Asp-rich carboxy-termil | | | | | | |
| domain 1 | | | | | | |
| platelet factor 4 | PF4 | (+) | | | | |
| ESTs | | (+) | | | | |
| expressed sequence AI553555 | AI553555 | (−) | | | | |
| tural killer tumor recognition | NKTR | (+) | | | | |
| sequence | | | | | | |
| expressed sequence AU019833 | C1orf24 | (+) | | | | |
| guanylate nucleotide binding protein 2 | GBP2 | (+) | (+) | RCC | C | |
| RIKEN cD 2310004L02 gene | FLJ10241 | (−) | | | | |
| ESTs | | (−) | | | | |
| expressed sequence C79732 | C79732 | (−) | | | | |
| Ras-GTPase-activating protein | G3BP2 | (+) | | | | |
| (GAP<120>) SH3-domain binding | | | | | | |
| protein 2 | | | | | | |
| glutathione S-transferase, theta 2 | GSTT2 | (−) | (−) | RCC | C | |
| CD52 antigen | CDW52 | (+) | (+) | RCC | C | |
| RIKEN cD 2810004N23 gene | 2810004N23Rik | (+) | | | | |
| ESTs | Rin3 | (+) | | | | |
| ESTs | | (+) | | | | |
| zinc finger protein 144 | ZNF144 | (+) | (−) | RCC | DC | |
| branched chain aminotransferase 2, | BCAT2 | (−) | | | | |
| mitochondrial | | | | | | |
| phenylalanine hydroxylase | PAH | (−) | (−) | RCC | C | |
| ESTs, Highly similar to T00268 | KIAA0597 | (−) | | | | |
| hypothetical protein KIAA0597 | | | | | | |
| (H. sapiens) | | | | | | |
| expressed sequence AV046379 | AV046379 | (−) | | | | |
| ribosomal protein L10A | RPL10A | (+) | (+) | RCC | C | |
| RIKEN cD 2410021P16 gene | MGC5601 | (−) | | | | |
| RIKEN cD 4632401C08 gene | 4632401C08Rik | (−) | | | | |
| BCL2-antagonist/killer 1 | BAK1 | (+) | | | | |
| myelocytomatosis oncogene | MYC | (+) | (+) | RCC | C | |
| guanosine diphosphate (GDP) | GDI-2 | (+) | | | | |
| dissociation inhibitor 3 | | | | | | |
| enoyl Coenzyme A hydratase, short | ECHS1 | (−) | | | | |
| chain, 1, mitochondrial | | | | | | |
| actin related protein ⅔ complex, | ARPC3 | (+) | (+) | RCC | C | (+) |
| subunit 3 (21 kDa) | | | | | | |
| retinol binding protein 1, cellular | RBP1 | (+) | | | | |
| solute carrier family 25 | SLC25A13 | (−) | | | | |
| (mitochondrial carrier | | | | | | |
| RIKEN cD 1100001F19 gene | UBE2D3 | (+) | | | | |
| constitutive photomorphogenic | COP1 | (+) | | | | |
| protein 1 (Arabidopsis) | | | | | | |
| ESTs, Weakly similar to AF182426 1 | | (−) | | | | |
| arylacetamide deacetylase | | | | | | |
| (R. norvegicus) | | | | | | |
| RIKEN cD 4930579A11 gene | VMP1 | (+) | (+) | RCC | C | |
| Mus musculus, clone MGC: 29021 | TAO1 | (+) | | | | |
| IMAGE: 3495957, mR, complete cds | | | | | | |
| expressed sequence C81457 | FLJ21022 | (−) | | | | |
| solute carrier family 25 | SLC25A19 | (−) | | | | |
| (mitochondrial deoxynucleotide | | | | | | |
| carrier), member 19 | | | | | | |
| protein S (alpha) | PROS1 | (+) | | | | |
| bone marrow stromal cell antigen 1 | BST1 | (+) | | | | |
| centrin 2 | CETN2 | (−) | | | | |
| RIKEN cD 3321401G04 gene | KIAA0738 | (+) | | | | |
| zuotin related factor 2 | ZRF1 | (+) | | | | |
| split hand/foot deleted gene 1 | DSS1 | (+) | (+) | RCC | C | |
| solute carrier family 1, member 1 | SLC1A1 | (+) | (−) | RCC | DC | |
| RIKEN cD 1110001I24 gene | BZW2 | (+) | | | | |
| glutaryl-Coenzyme A dehydrogese | GCDH | (−) | | | | |
| RIKEN cD 4921528E07 gene | 4921528E07Rik | (+) | | | | |
| RIKEN cD 1810013B01 gene | 1810013B01Rik | (−) | | | | |
| expressed sequence AU042434 | AU042434 | (+) | | | | |
| Mus musculus, Similar to CGI-147 | | (+) | | | | |
| protein, clone MGC: 25743 | | | | | | |
| IMAGE: 3990061, mR, complete cds | | | | | | |
| ubiquitin specific protease 7 | USP7 | (+) | | | | |
| (expressed sequence AA409944) | | | | | | |
| N-acetylneuramite pyruvate lyase | C1orf13 | (+) | | | | |
| L-3-hydroxyacyl-Coenzyme A | HADHSC | (−) | (−) | RCC | C | |
| dehydrogese, short chain | | | | | | |
| major vault protein | MVP | (+) | | | | |
| growth arrest specific 2 | GAS2 | (−) | (−) | RCC | C | |
| RIKEN cD 1110002C08 gene | MGC9564 | (−) | | | | |
| acetyl-Coenzyme A transporter | ACATN | (−) | | | | |
| RIKEN cD 5133400A03 gene | 5133400A03Rik | (+) | | | | |
| ALL1-fused gene from chromosome | AF1Q | (−) | | | | |
| 1q | | | | | | |
| myosin Ic | MYO1C | (+) | | | | |
| ESTs | | (−) | | | | |
| NCK-associated protein 1 | NCKAP1 | (+) | | | | |
| integrin alpha 6 | ITGA6 | (+) | (+) | RCC | C | |
| Mus musculus LDLR dan mR, | | (−) | | | | |
| complete cds | | | | | | |
| RIKEN cD 1110032A13 gene | FLJ21172 | (+) | | | | |
| metastasis associated 1-like 1 | MTA1L1 | (+) | | | | |
| fibulin 5 | FBLN5 | (−) | | | | |
| expressed sequence C85317 | C85317 | (+) | | | | |
| ESTs | | (+) | | | | |
| crystallin, lamda 1 | CRYL1 | (−) | | | | |
| RIKEN cD 1700016A15 gene | FLJ11806 | (+) | | | | |
| 5-azacytidine induced gene 1 | Azi1 | (−) | | | | |
| estrogen related receptor, alpha | ESRRA | (−) | | | | |
| spermatogenesis associated factor | SPATA5 | (+) | | | | |
| RIKEN cD 4930533K18 gene | | (+) | | | | |
| Harvey rat sarcoma oncogene, | RRAS | (+) | | | | |
| subgroup R | | | | | | |
| complement component 1, q | C1QB | (+) | (+) | RCC | C | |
| subcomponent, beta polypeptide | | | | | | |
| S-adenosylhomocysteine hydrolase | AHCY | (−) | (−) | RCC | C | |
| brain protein 44-like | BRP44l | (−) | (−) | RCC | C | |
| inositol polyphosphate-5- | INPP5B | (−) | | | | |
| phosphatase, 75 kDa | | | | | | |
| hyaluronic acid binding protein 2 | HABP2 | (−) | | | | |
| syndecan 1 | SDC1 | (+) | (−) | RCC | DC | |
| guanosine monophosphate reductase | GMPR | (+) | | | | |
| alcohol dehydrogese 4 (class II), pi | ADH4 | (−) | (−) | RCC | C | |
| polypeptide | | | | | | |
| branched chain ketoacid dehydrogese | BCKDHA | (−) | (+) | RCC | DC | |
| E1, alpha polypeptide | | | | | | |
| ESTs, Weakly similar to brain- | | (−) | | | | |
| specific angiogenesis inhibitor 1- | | | | | | |
| associated protein 2 (Mus musculus) | | | | | | |
| (M. musculus) | | | | | | |
| Unknown | | (−) | | | | |
| R binding motif protein 3 | RBM3 | (+) | | | | |
| superoxide dismutase 2, | SOD2 | (−) | (+) | RCC | DC | (+) |
| mitochondrial | | | | | | |
| histone deacetylase 1 | HDAC1 | (+) | | | | (+) |
| biglycan | BGN | (+) | | | | |
| ras homolog 9 (RhoC) | ARHC | (+) | | | | |
| latexin | LXN | (+) | (+) | RCC | C | |
| pyruvate kise 3 | PKM2 | (+) | | | | (+) |
| SMC (structural maintence of | SMC1L1 | (+) | (−) | RCC | DC | |
| chromosomes 1)-like 1 (S. cerevisiae) | | | | | | |
| serum/glucocorticoid regulated kise 2 | SGK2 | (−) | | | | |
| WD repeat domain 1 | WDR1 | (+) | | | | |
| RIKEN cD 2310001A20 gene | C20orf3 | (−) | | | | |
| thymidine kise 1 | TK1 | (+) | (+) | RCC | C | |
| glutathione S-transferase, alpha 4 | GSTA4 | (−) | | | | |
| PH domain containing protein in reti 1 | PHRET1 | (−) | | | | |
| RIKEN cD 1110020L19 gene | TREX2 | (+) | | | | |
| tumor necrosis factor receptor | TNFRSF1B | (+) | | | | |
| superfamily, member 1b | | | | | | |
| UDP-Gal:betaGlcc beta 1,4- | B4GALT2 | (+) | | | | |
| galactosyltransferase, polypeptide 2 | | | | | | |
| N-myc downstream regulated 2 | NDRG2 | (−) | | | | (+) |
| platelet derived growth factor, alpha | PDGFA | (+) | | | | |
| hemochromatosis | HFE | (+) | | | | |
| serine protease inhibitor, Kunitz type 2 | SPINT2 | (+) | | | | |
| CD53 antigen | CD53 | (+) | (+) | RCC | C | |
| leucine zipper-EF-hand containing | LETM1 | (−) | | | | |
| transmembrane protein 1 | | | | | | |
| Mus musculus, Similar to xylulokise | | (−) | | | | |
| homolog (H. influenzae), clone | | | | | | |
| IMAGE: 5043428, mR, partial cds | | | | | | |
| expressed sequence AW261723 | SLC17A3 | (−) | | | | |
| phytanoyl-CoA hydroxylase | PHYH | (−) | (−) | RCC | C | |
| RIKEN cD 2610511O17 gene | FLJ20272 | (+) | | | | |
| RIKEN cD 2610306D21 gene | ANAPC4 | (+) | | | | |
| ESTs | FLJ22184 | (−) | | | | |
| adaptor-related protein complex AP- | AP3S1 | (+) | (+) | RCC | C | |
| 3, sigma 1 subunit | | | | | | |
| Mus musculus, Similar to | MGC4368 | (−) | | | | |
| hypothetical protein MGC4368, clone | | | | | | |
| MGC: 28978 IMAGE: 4503381, mR, | | | | | | |
| complete cds | | | | | | |
| phenylalkylamine Ca2+ antagonist | EBP | (−) | | | | |
| (emopamil) binding protein | | | | | | |
| MORF-related gene X | MORF4L2 | (+) | (+) | RCC | C | |
| AU R binding protein/enoyl- | AUH | (−) | | | | |
| coenzyme A hydratase | | | | | | |
| SWI/SNF related, matrix associated, | SMARCE1 | (+) | (+) | RCC | C | |
| actin dependent regulator of | | | | | | |
| chromatin, subfamily e, member 1 | | | | | | |
| RIKEN cD 1810054O13 gene | 1810054O13Rik | (−) | | | | |
| spermidine/spermine N1-acetyl | SAT | (+) | | | | (+) |
| transferase | | | | | | |
| v-ral simian leukemia viral oncogene | RALA | (+) | (+) | RCC | C | |
| homolog A (ras related) | | | | | | |
| Mus musculus, clone MGC: 37818 | MGC37818 | (−) | | | | |
| IMAGE: 5098655, mR, complete cds | | | | | | |
| expressed sequence AI117581 | AI117581 | (−) | | | | |
| RIKEN cD 6230410I01 gene | FLJ10849 | (+) | | | | |
| RIKEN cD 2310075M15 gene | 2310075M15Rik | (+) | | | | |
| RIKEN cD 0610025I19 gene | 0610025I19Rik | (−) | | | | |
| expressed sequence AI118577 | ZNF14 | (−) | | | | |
| neuropilin | NRP1 | (+) | (+) | RCC | C | |
| G-rich RNA sequence binding factor | GRSF1 | (−) | (+) | RCC | DC | (+) |
| 1 (D5Wsu31e) D segment, Chr 5, | | | | | | |
| Wayne State University 31, expressed | | | | | | |
| solute carrier family 13 (sodium- | SLC13A3 | (−) | (−) | RCC | C | |
| dependent dicarboxylate transporter), | | | | | | |
| member 3 | | | | | | |
| ubiquitin-like 1 (sentrin) activating | UBA2 | (+) | | | | |
| enzyme E1B | | | | | | |
| RIKEN cD 1500041J02 gene | FLJ13448 | (−) | | | | |
| D segment, Chr 8, Brigham & | D8Bwg1320e | (−) | | | | |
| Women's Genetics 1320 expressed | | | | | | |
| expressed sequence C86302 | C86302 | (+) | | | | |
| expressed sequence AI987692 | AI987692 | (+) | | | | |
| parvalbumin | PVALB | (−) | (+)/(−) | RCC | conflict | |
| small nuclear ribonucleoprotein E | SNRPE | (+) | (+) | RCC | C | |
| RIKEN cD 6530411B15 gene | DKFZp564K1964.1 | (−) | | | | |
| MARCKS-like protein | MLP | (+) | | | | |
| ras homolog D (RhoD) | ARHD | (+) | | | | |
| Mus musculus, clone | C13orf11 | (−) | | | | |
| IMAGE: 3967158, mR, partial cds | | | | | | |
| RIKEN cD 1700037H04 gene | FLJ20550 | (+) | | | | |
| deiodise, iodothyronine, type I | DIO1 | (−) | | | | |
| RIKEN cD 060011C19 gene | FLJ22386 | (−) | | | | |
| v-ral simian leukemia viral oncogene | RALB | (+) | | | | |
| homolog B (ras related) | | | | | | |
| ESTs, Weakly similar to MAJOR | | (−) | | | | |
| URIRY PROTEIN 4 PRECURSOR | | | | | | |
| (M. musculus) | | | | | | |
| protein C | PROC | (−) | (−) | RCC | C | |
| alpha-methylacyl-CoA racemase | AMACR | (−) | (+) | RCC | DC | |
| RIKEN cD 2810411G23 gene | TPD52L2 | (+) | (+) | RCC | C | |
| Unknown | | (−) | | | | |
| DJ (Hsp40) homolog, subfamily A, | DNAJA1 | (−) | | | | |
| member 1 | | | | | | |
| RIKEN cD 1200003E16 gene | 1200003E16Rik | (−) | | | | |
| heterogeneous nuclear | HNRPA1 | (+) | (+) | RCC | C | |
| ribonucleoprotein A1 | | | | | | |
| FK506 binding protein 1a (12 kDa) | FKBP1A | (+) | | | | (+) |
| RIKEN cD 4933405K01 gene | MGC14799 | (+) | | | | |
| surfeit gene 4 | SURF4 | (+) | (+) | RCC | C | |
| mitogen activated protein kise 13 | MAPK13 | (+) | | | | |
| RIKEN cD 2310022K15 gene | KLHDC2 | (+) | | | | |
| RIKEN cD 1300002P22 gene | ECH1 | (−) | | | | |
| ectonucleotide | ENPP2 | (−) | (+) | RCC | DC | |
| pyrophosphatase/phosphodiesterase 2 | | | | | | |
| PCTAIRE-motif protein kise 3 | PCTK3 | (−) | (+) | RCC | DC | |
| splicing factor 3b, subunit 1, 155 kDa | SF3B1 | (+) | (+) | RCC | C | |
| zinc finger protein 36, C3H type-like 2 | ZFP36L2 | (+) | | | | |
| M. musculus mR for protein expressed | Tex2 | (−) | | | | |
| at high levels in testis | | | | | | |
| nuclear receptor coactivator 4 | NCOA4 | (−) | (+) | RCC | DC | |
| PC4 and SFRS1 interacting protein 2 | PSIP2 | (+) | | | | |
| (expressed sequence AU015605) | | | | | | |
| purinergic receptor (family A group | P2RY5 | (+) | | | | |
| 5); RIKEN cD 2610302I02 gene | | | | | | |
| ESTs, Moderately similar to SEC7 | | (−) | | | | |
| homolog (Homo sapiens) (H. sapiens) | | | | | | |
| Mus musculus, clone | G630055P03Ri | (+) | | | | |
| IMAGE: 4456744, mR, partial cds | | | | | | |
| Blu protein | ZMYND10 | (−) | | | | |
| solute carrier family 6 | SLC6A9 | (+) | | | | |
| (neurotransmitter transporter, | | | | | | |
| glycine), member 9/glycine | | | | | | |
| transporter 1 | | | | | | |
| Mus musculus, Similar to MIPP65 | 1500032D16Rik | (−) | | | | |
| protein, clone MGC: 18783 | | | | | | |
| IMAGE: 4188234, mR, complete cds | | | | | | |
| expressed sequence AU018056 | AU018056 | (−) | | | | |
| RIKEN cD 1810009M01 gene | LR8 | (+) | | | | |
| serum/glucocorticoid regulated kise | SGK | (−) | | | | |
| Mus musculus, Similar to unc93 | UNC93B1 | (+) | | | | |
| (C. elegans) homolog B, clone | | | | | | |
| MGC: 25627 IMAGE: 4209296, mR, | | | | | | |
| complete cds | | | | | | |
| RIKEN cD 2810473M14 gene | 2810473M14Rik | (−) | | | | |
| TATA box binding protein-like | TBPL1 | (+) | | | | |
| protein | | | | | | |
| acyl-Coenzyme A dehydrogese, | ACADSB | (−) | (−) | RCC | C | |
| short/branched chain | | | | | | |
| Mus musculus, clone MGC: 12159 | D530037I19Rik | (+) | | | | |
| IMAGE: 3711169, mR, complete cds | | | | | | |
| proline dehydrogese | PRODH | (−) | | | | (+) |
| leukemia-associated gene | STMN1 | (+) | (+) | RCC | C | |
| Mus musculus evectin-2 (Evt2) mR, | PLEKHB2 | (−) | | | | |
| complete cds | | | | | | |
| kise insert domain protein receptor | KDR | (−) | (+) | RCC | DC | |
| RIKEN cD 1300019I21 gene | MTAP | (+) | | | | |
| slit homolog 3 (Drosophila) | SLIT3 | (+) | | | | |
| RIKEN cD 6330565B14 gene | ADH8 | (−) | | | | |
| RIKEN cD 1810043O07 gene | KIAA0601 | (+) | | | | |
| RIKEN cD 1110008B24 gene | C14orf111 | (+) | | | | |
| thyroid hormone responsive SPOT14 | THRSP | (−) | | | | |
| homolog (Rattus) | | | | | | |
| RIKEN cD 2310079C17 gene | DKFZP547E2110 | (+) | | | | |
| intergral membrane protein 1 | ITM1 | (+) | | | | |
| expressed sequence R75232 | R75232 | (+) | | | | |
| coronin, actin binding protein 1B | CORO1B | (+) | (−) | RCC | DC | |
| RIKEN cD 2310004I03 gene | 2310004I03Rik | (−) | | | | |
| RIKEN cD 1010001M04 gene | 1010001M04Rik | (−) | | | | |
| RIKEN cD 2700038M07 gene - | WSB1 | (+) | (−) | RCC | DC | |
| pending | | | | | | |
| RIKEN cD 1100001J13 gene - | KIAA1049 | (−) | (+) | RCC | DC | |
| pending | | | | | | |
| RIKEN cD 0610016J10 gene | CGI-27 | (+) | | | | |
| SET translocation | SET | (+) | (+) | RCC | C | (+) |
| ESTs, Highly similar to prefoldin 4 | PFDN4 | (+) | (+) | RCC | C | |
| (Homo sapiens) (H. sapiens) | | | | | | |
| Mus musculus, Similar to nucleolar | HSA6591 | (+) | (+) | RCC | C | |
| cysteine-rich protein, clone | | | | | | |
| MGC: 6718 IMAGE: 3586161, mR, | | | | | | |
| complete cds - pending | | | | | | |
| Mus musculus, Similar to sirtuin | SIRT7 | (−) | | | | |
| silent mating type information | | | | | | |
| regulation 2 homolog 7 (S. cerevisiae), | | | | | | |
| clone MGC: 37560 | | | | | | |
| IMAGE: 4987746, mR, complete cds | | | | | | |
| Mus musculus, clone MGC: 36554 | D14Ertd226e | (+) | | | | |
| IMAGE: 4954874, mR, complete cds | | | | | | |
| RIKEN cD 2610206D03 gene | 2610206D03Rik | (+) | | | | |
| peroxisomal delta3, delta2-enoyl- | PECI | (−) | (−) | RCC | C | |
| Coenzyme A isomerase | | | | | | |
| (Sdccagg28) serologically defined | STARD10 | (−) | | | | |
| colon cancer antigen 28 | | | | | | |
| protein tyrosine phosphatase 4a1 | PTP4A1 | (+) | | | | |
| peroxisomal biogenesis factor 13 | PEX13 | (−) | | | | |
| ESTs | | (−) | | | | |
| expressed sequence AI957255 | KIAA0564 | (−) | | | | |
| cleavage and polyadenylation specific | CPSF5 | (+) | | | | |
| factor 5, 25 kD subunit | | | | | | |
| intercellular adhesion molecule | ICAM1 | (+) | (+) | RCC | C | (+) |
| RIKEN cD 1200013A08 gene | MGC3047 | (+) | | | | |
| D primase, p49 subunit | PRIM1 | (+) | | | | |
| RIKEN cD 2410029D23 gene | ATP6V1E1 | (−) | | | | |
| RIKEN cD 1300017C12 gene | FLJ10948 | (−) | (−) | RCC | C | |
| steroid receptor R activator 1 | SRA1 | (+) | | | | |
| regulator for ribosome resistance | RRS1 | (+) | | | | |
| homolog (S. cerevisiae) | | | | | | |
| RIKEN cD 0610006N12 gene | NDUFB4 | (−) | | | | |
| poly(rC) binding protein 1 | PCBP1 | (+) | (+) | RCC | C | |
| expressed sequence AU015645 | AU015645 | (−) | | | | |
| ESTs | | (+) | | | | |
| Mus musculus mR for alpha-albumin | AFM | (−) | (−) | RCC | C | |
| protein | | | | | | |
| small nuclear ribonucleoprotein D2 | SNRPD2 | (+) | (+) | RCC | C | |
| succinate dehydrogenase complex, | SDHB | (−) | (−) | RCC | C | |
| subunit B, iron sulfur (Ip); RIKEN cD | | | | | | |
| 0710008N11 gene | | | | | | |
| homocysteine-inducible, endoplasmic | HERPUD1 | (−) | | | | |
| reticulum stress-inducible, ubiquitin- | | | | | | |
| like domain member 1 | | | | | | |
| solute carrier family 16 | SLC16A7 | (−) | (+) | RCC | DC | |
| (monocarboxylic acid transporters), | | | | | | |
| member 7 | | | | | | |
| activity-dependent neuroprotective | ADNP | (+) | | | | |
| protein | | | | | | |
| RIKEN cD 1810027P18 gene | DCXR | (−) | (−) | RCC | C | |
| insulin-like growth factor binding | IGFBP3 | (−) | (+) | RCC | DC | (+) |
| protein 3 | | | | | | |
| smoothened homolog (Drosophila) | SMOH | (−) | | | | |
| SEC13 related gene (S. cerevisiae) | SEC13L1 | (+) | | | | |
| RIKEN cD 1110003H02 gene | | | | | | |
| Mus musculus, Similar to | FLJ10883 | (−) | | | | |
| chromosome 20 open reading frame | | | | | | |
| 36, clone IMAGE: 5356821, mR, | | | | | | |
| partial cds | | | | | | |
| flotillin 1 | FLOT1 | (+) | | | | |
| RIKEN cD 2700055K07 gene | CGI-38 | (+) | | | | |
| matrix metalloproteise 23 | MMP23A | (+) | | | | |
| Mus musculus, Similar to KIAA1075 | TENC1 | (−) | | | | |
| protein, clone IMAGE: 5099327, mR, | | | | | | |
| partial cds | | | | | | |
| RIKEN cD 1110007F23 gene | 1110007F23Rik | (+) | | | | |
| glycine N-methyltransferase | GNMT | (−) | | | | |
| zinc finger like protein 1 | ZFPL1 | (−) | | | | |
| capping protein beta 1 | CAPZB | (+) | | | | |
| RIKEN cD 6720463E02 gene | | (+) | | | | |
| expressed sequence AA408783 | SPEC2 | (+) | (+) | RCC | C | |
| elongation of very long chain fatty | ELOVL1 | (+) | | | | |
| acids (FEN1/Elo2, SUR4/Elo3, | | | | | | |
| yeast)-like 1 | | | | | | |
| carnitine palmitoyltransferase 2 | CPT2 | (−) | (−) | RCC | C | |
| Mus musculus, Similar to | D14Ertd813e | (+) | | | | |
| hypothetical protein FLJ20335, clone | | | | | | |
| MGC: 28912 IMAGE: 4922274, mR, | | | | | | |
| complete cds | | | | | | |
| flap structure specific endonuclease 1 | FEN1 | (+) | (+) | RCC | C | |
| chloride intracellular channel 1 | CLIC1 | (+) | (+) | RCC | C | |
| ATPase, H+ transporting, V1 subunit | ATP6V1F | (−) | | | | |
| F; RIKEN cD 1110004G16 gene | | | | | | |
| BRG1/brm-associated factor 53A | BAF53A | (+) | | | | |
| matrix metalloproteise 2 | MMP2 | (+) | (−) | RCC | DC | (+) |
| methylenetetrahydrofolate | MTHFD1 | (−) | (+) | RCC | DC | |
| dehydrogese (DP+ dependent), | | | | | | |
| methenyltetrahydrofolate | | | | | | |
| cyclohydrolase, | | | | | | |
| formyltetrahydrofolate synthase | | | | | | |
| damage specific D binding protein 1 | DDB1 | (+) | | | | |
| (127 kDa) | | | | | | |
| glutathione transferase zeta 1 | GSTZ1 | (−) | | | | |
| (maleylacetoacetate isomerase) | | | | | | |
| isocitrate dehydrogese 2 (DP+), | IDH2 | (−) | | | | |
| mitochondrial | | | | | | |
| ubiquitin-like 1 (sentrin) activating | SAE1 | (+) | (+) | RCC | C | |
| enzyme E1A | | | | | | |
| actin, beta, cytoplasmic | ACTB | (+) | (+) | RCC | C | |
| lectin, galactose binding, soluble 3 | LGALS3 | (+) | (+) | RCC | C | |
| upregulated during skeletal muscle | MGC14697 | (−) | | | | |
| growth 5 | | | | | | |
| polycystic kidney disease 1 homolog | PKD1 | (−) | (+) | RCC | DC | (+) |
| Mus musculus, Similar to | SF3b10 | (+) | | | | |
| hypothetical protein MGC3133, clone | | | | | | |
| MGC: 11596 IMAGE: 3965951, mR, | | | | | | |
| complete cds | | | | | | |
| RIKEN cD 1700015P13 gene | 1700015P13Rik | (−) | | | | |
| MYC-associated zinc finger protein | MAZ | (+) | (+) | RCC | C | |
| (purine-binding transcription factor) | | | | | | |
| proteasome (prosome, macropain) | PSMD13 | (+) | (+) | RCC | C | |
| 26S subunit, non-ATPase, 13 | | | | | | |
| pyruvate dehydrogese 2 | PDK2 | (−) | | | | |
| ATPase, H+ transporting, lysosomal | ATP6V1A1 | (−) | | | | (+) |
| (vacuolar proton pump), alpha 70 kDa, | | | | | | |
| isoform 1 | | | | | | |
| N-acetylglucosamine kise | NAGK | (+) | (+) | RCC | C | |
| arginine-rich, mutated in early stage | ARMET | (+) | | | | |
| tumors | | | | | | |
| sigling intermediate in Toll pathway- | Sitpec | (−) | (−) | RCC | C | |
| evolutiorily conserved | | | | | | |
| cell division cycle 25 homolog A | CDC25A | (+) | | | | |
| (S. cerevisiae) | | | | | | |
| B-box and SPRY domain containing | BSPRY | (+) | | | | |
| Mus musculus, clone MGC: 6545 | MAT2A | (−) | (+) | RCC | DC | |
| IMAGE: 2655444, mR, complete cds | | | | | | |
| expressed sequence C86169 | C86169 | (−) | | | | |
| immunoglobulin superfamily, | IGSF8 | (+) | | | | |
| member 8 | | | | | | |
| RIKEN cD 2410002J21 gene | ENIGMA | (+) | | | | (+) |
| myeloid-associated differentiation | MYADM | (+) | | | | |
| marker | | | | | | |
| RIKEN cD 5031412I06 gene | Dutp | (+) | | | | |
| RIKEN cD 2310032J20 gene | BDH | (−) | | | | |
| serine hydroxymethyl transferase 2 | SHMT2 | (−) | (+) | RCC | DC | |
| (mitochondrial); RIKEN cD | | | | | | |
| 2700043D08 gene | | | | | | |
| ribosomal protein L21 | RPL21 | (+) | (+) | RCC | C | (+) |
| thioether S-methyltransferase | Temt | (−) | | | | |
| interferon inducible protein 1 | Ifi1 | (−) | | | | |
| Hprt | HPRT1 | (+) | | | | |
| retinoblastoma-like 1 (p107) | RBL1 | (+) | | | | |
| RAB3D, member RAS oncogene | RAB3D | (+) | | | | |
| family | | | | | | |
| glycine amidinotransferase (L- | GATM | (−) | (−) | RCC | C | |
| arginine:glycine amidinotransferase) | | | | | | |
| ribosomal protein S23 | RPS23 | (+) | (+) | RCC | C | |
| expressed sequence C87222 | C87222 | (+) | | | | |
| RIKEN cD 1300013F15 gene | FLJ22390 | (−) | | | | |
| erythrocyte protein band 4.1/Mus | EPB41 | (−) | (−) | RCC | C | |
| musculus adult male tongue cD, | | | | | | |
| RIKEN full-length enriched library, | | | | | | |
| clone: 2310065B16: erythrocyte | | | | | | |
| protein band 4.1, full insert sequence | | | | | | |
| RIKEN cD 5730406I15 gene | KIAA0102 | (+) | | | | |
| mitochondrial ribosomal protein L50; | MRPL50 | (−) | | | | |
| (D4Wsu125e) D segment, Chr 4, | | | | | | |
| Wayne State University 125, | | | | | | |
| expressed | | | | | | |
| myristoylated alanine rich protein | MACS | (+) | | | | |
| kise C substrate | | | | | | |
| ribosomal protein L8 | RPL8 | (+) | (+) | RCC | C | |
| lysosomal-associated protein | LAPTM4A | (+) | | | | |
| transmembrane 4A | | | | | | |
| Mus musculus, clone MGC: 19042 | OGDH | (−) | | | | |
| IMAGE: 4188988, mR, complete cds | | | | | | |
| RIKEN cD 1810058K22 gene | CDC42EP1 | (+) | | | | |
| Mus musculus, Similar to dendritic | GA17 | (+) | | | | |
| cell protein, clone MGC: 11741 | | | | | | |
| IMAGE: 3969335, mR, complete cds | | | | | | |
| eukaryotic translation initiation factor | EIF3S4 | (+) | (+) | RCC | C | |
| 3, subunit 4 (delta, 44 kDa) | | | | | | |
| RIKEN cD 2510015F01 gene | FLJ12442 | (+) | | | | |
| nuclear protein 15.6 | P17.3 | (−) | | | | |
| glucose-6-phosphatase, transport | G6PT1 | (−) | | | | |
| protein 1 | | | | | | |
| solute carrier family 22 (organic | SLC22A6 | (−) | (−) | RCC | C | |
| anion transporter), member 6 | | | | | | |
| expressed sequence AI132189 | AI132189 | (−) | | | | |
| coagulation factor XIII, beta subunit | F13B | (−) | | | | |
| TEA domain family member 2 | TEAD2 | (+) | | | | |
| casein kise 1, epsilon | CSNK1E | (+) | | | | |
| ESTs | | (−) | | | | |
| proteasome (prosome, macropain) | PSMA6 | (+) | (+) | RCC | C | |
| subunit, alpha type 6 | | | | | | |
| syntrophin, basic 2 | SNTB2 | (+) | | | | |
| ubiquitin-conjugating enzyme E2N | UBE2N | (+) | | | | |
| Mus musculus, clone | | (−) | | | | |
| IMAGE: 3589087, mR, partial cds | | | | | | |
| D segment, Chr 18, Wayne State | ALDH7A1 | (−) | (−) | RCC | C | |
| University 181, expressed | | | | | | |
| Kruppel-like factor 5 | KLF5 | (+) | (+) | RCC | C | |
| X transporter protein 2 | Xtrp2 | (−) | | | | |
| CDC28 protein kise 1 | CKS1B | (+) | (+) | RCC | C | |
| expressed sequence AI461788 | AI461788 | (+) | | | | |
| phosphatidylinositol 3-kise, | PIK3R1 | (+) | | | | |
| regulatory subunit, polypeptide 1 | | | | | | |
| (p85 alpha) | | | | | | |
| sex-lethal interactor homolog | RPC5 | (−) | | | | |
| (Drosophila) | | | | | | |
| expressed sequence AW124722 | AW124722 | (−) | | | | |
| ubiquitin-conjugating enzyme E2L 3 | UBE2L3 | (+) | | | | |
| expressed sequence AI836219 | AI836219 | (−) | | | | |
| ESTs, Weakly similar to TS13 | MGC39016 | (+) | | | | |
| MOUSE TESTIS-SPECIFIC | | | | | | |
| PROTEIN PBS13 (M. musculus) | | | | | | |
| expressed sequence AI480660 | AI480660 | (−) | | | | |
| ribosomal protein L19 | RPL19 | (+) | (+) | RCC | C | |
| Mus musculus, clone MGC: 12039 | Itpr5 | (−) | | | | |
| IMAGE: 3603661, mR, complete cds | | | | | | |
| inhibin beta-B | INHBB | (+) | (+) | RCC | C | |
| serine (or cysteine) proteise inhibitor, | SERPINE2 | (+) | | | | |
| clade E (nexin, plasminogen activator | | | | | | |
| inhibitor type 1), member 2 | | | | | | |
| ESTs | | (+) | | | | |
| dihydropyrimidise | DPYS | (−) | (−) | RCC | C | |
| glutathione S-transferase, mu 6 | GSTM1 | (+) | | | | |
| PYRIN-containing APAF1-like | PYPAF5 | (−) | | | | |
| protein 5/expressed sequence | | | | | | |
| AI504961 | | | | | | |
| RIKEN cD 1200011D11 gene | BK65A6.2 | (−) | | | | |
| kinectin 1 | KTN1 | (+) | | | | |
| ribosomal protein L28 | RPL28 | (+) | (+) | RCC | C | |
| ESTs | | (+) | | | | |
| four and a half LIM domains 1 | FHL1 | (−) | (+) | RCC | DC | (+) |
| phosphatidylinositol transfer protein | PITPN | (+) | | | | |
| growth differentiation factor 15 | PLAB | (+) | (+) | RCC | C | (+) |
| ESTs | | (−) | | | | |
| expressed sequence AI646725 | MDS028 | (−) | | | | |
| insulin-like growth factor binding | IGFALS | (−) | | | | |
| protein, acid labile subunit | | | | | | |
| carboxypeptidase E | CPE | (+) | | | | |
| peptidylprolyl isomerase C-associated | LGALS3BP | (+) | (+) | RCC | C | |
| protein | | | | | | |
| vascular endothelial growth factor A | VEGF | (−) | (+) | RCC | DC | (+) |
| expressed sequence AI465301 | AI465301 | (−) | | | | |
| malate dehydrogese, soluble | MDH1 | (−) | | | | |
| potassium channel, subfamily K, | KCNK2 | (−) | | | | |
| member 2 | | | | | | |
| ribosomal protein, large, P1 | RPLP1 | (+) | (+) | RCC | C | |
| expressed sequence AI448003 | AI448003 | (+) | | | | |
| expressed sequence AI504062 | AI504062 | (+) | | | | |
| poly (A) polymerase alpha | PAPOLA | (−) | (+) | RCC | DC | |
| DPH oxidase 4 | NOX4 | (−) | (?) | RCC | conflict | |
| small inducible cytokine subfamily D, 1 | SCYD1 | (+) | | | | |
| secreted phosphoprotein 1 | SPP1 | (+) | (−)/(+) | RCC | conflict | |
| ESTs | | (−) | | | | |
| ESTs | | (−) | | | | |
| AMP deamise 3 | AMPD3 | (+) | | | | |
| glycerol kise | GK | (−) | (−) | RCC | C | |
| J domain protein 1 | JDP1 | (−) | | | | |
| Mus musculus, clone | LOC224650 | (−) | | | | |
| IMAGE: 3155544, mR, partial cds | | | | | | |
| RIKEN cD 1110038L14 gene | CKS2 | (+) | (+) | RCC | C | |
| cornichon homolog (Drosophila) | CNIH | (+) | | | | |
| ubiquitin-conjugating enzyme E2I | UBE2I | (+) | | | | (+) |
| Bcl-2-related ovarian killer protein | BOK | (+) | | | | |
| tyrosine 3-monooxygese/tryptophan | YWHAH | (+) | (+) | RCC | C | |
| 5-monooxygese activation protein, eta | | | | | | |
| polypeptide | | | | | | |
| (Gus-s) beta-glucuronidase structural | GUSB | (+) | | | | |
| RIKEN cD A930008K15 gene | KIAA0605 | (−) | | | | |
| myosin light chain, alkali, nonmuscle | MYL6 | (+) | (−) | RCC | DC | |
| apolipoprotein B editing complex 1 | APOBEC1 | (+) | | | | |
| soc-2 (suppressor of clear) homolog | SHOC2 | (+) | | | | |
| (C. elegans) | | | | | | |
| RIKEN cD 1200016G03 gene | 1200016G03Rik | (−) | | | | |
| ESTs | 9130203F04Rik | (+) | | | | |
| hydroxysteroid dehydrogese-3, | Hsd3b3 | (−) | | | | |
| delta<5>-3-beta | | | | | | |
| expressed sequence AI507121 | AI507121 | (−) | | | | |
| claudin 1 | CLDN1 | (+) | (+) | RCC | C | |
| serine protease inhibitor 6 | SERPINB9 | (+) | | | | |
| small inducible cytokine A5 | SCYA5 | (+) | (+) | RCC | C | |
| serine hydroxymethyl transferase 1 | SHMT1 | (−) | (+) | RCC | DC | |
| (soluble) | | | | | | |
| RIKEN cD 3021401A05 gene | 3021401A05Rik | (+) | | | | |
| ESTs | | (−) | | | | |
| Tnf receptor-associated factor 2 | TRAF2 | (+) | | | | |
| talin 2 | TLN2 | (−) | | | | |
| high mobility group box 3 | HMGB3 | (+) | (+) | RCC | C | |
| RIKEN cD 1700012B18 gene | OKL38 | (−) | | | | |
| ornithine decarboxylase, structural | ODC1 | (+) | | | | |
| gap junction membrane channel | GJB2 | (−) | (+) | RCC | DC | |
| protein beta 2 | | | | | | |
| solute carrier family 2 (facilitated | SLC2A5 | (−) | (−) | RCC | C | |
| glucose transporter), member 5 | | | | | | |
| ESTs, Moderately similar to T08673 | KIAA0977 | (−) | (−) | RCC | C | |
| hypothetical protein | | | | | | |
| DKFZp564C0222.1 (H. sapiens) | | | | | | |
| nuclear factor of kappa light chain | NFKB1 | (+) | | | | |
| gene enhancer in B-cells 1, p105 | | | | | | |
| Williams-Beuren syndrome | WBSCR14 | (−) | (−) | RCC | C | |
| chromosome region 14 homolog | | | | | | |
| (human) | | | | | | |
| RIKEN cD 1300018I05 gene | KIAA0082 | (+) | | | | |
| RIKEN cD 1110005N04 gene | TAF5L | (+) | | | | |
| caspase 3, apoptosis related cysteine | CASP3 | (+) | | | | (−) |
| protease | | | | | | |
| glycoprotein 49 B | Gp49b | (+) | | | | |
| histocompatibility 2, Q region locus 7 | H2-Q7 | (+) | | | | |
| ESTs | | (+) | | | | |
| cyclin-dependent kise inhibitor 1A | CDKN1A | (+) | (+)/(+??) | RCC | conflict | (+) |
| (P21) | | | | | | |
| Rho guanine nucleotide exchange | ARHGEF3 | (−) | | | | |
| factor (GEF) 3 | | | | | | |
| complement component 1, q | C1QG | (+) | | | | |
| subcomponent, c polypeptide | | | | | | |
| RIKEN cD 9530058B02 gene | MGC15416 | (−) | | | | |
| D segment, Chr 17, ERATO Doi 441, | D17Ertd441e | (+) | | | | |
| expressed | | | | | | |
| expressed sequence AI844685 | MGC15429 | (−) | | | | |
| slit homolog 2 (Drosophila) | SLIT2 | (−) | | | | |
| tetranectin (plasminogen binding | T | (−) | | | | |
| protein) | | | | | | |
| citrate lyase beta like | CLYBL | (−) | | | | |
| succite-Coenzyme A ligase, GDP- | SUCLG2 | (−) | | | | (+) |
| forming, beta subunit | | | | | | |
| cytokine inducible SH2-containing | SOCS3 | (+) | | | | |
| protein 3 | | | | | | |
| solute carrier family 4 (anion | SLC4A4 | (−) | (−) | RCC | C | |
| exchanger), member 4 | | | | | | |
| heat shock protein, 105 kDa | HSPH1 | (−) | (+) | RCC | DC | |
| RIKEN cD 4733401N12 gene | CPSF6 | (+) | | | | |
| ESTs | | (−) | | | | |
| ribosomal protein L3 | RPL3 | (+) | | | | (+) |
| carnitine palmitoyltransferase 1, | CPT1B | (−) | | | | |
| muscle | | | | | | |
| ESTs | | (+) | | | | |
| RIKEN cD 2310010G13 gene | 2310010G13Rik | (−) | | | | |
| ESTs | | (−) | | | | |
| expressed sequence AI558103 | LRRN1 | (−) | | | | |
| Unknown | | (−) | | | | |
| RIKEN cD 4932442K08 gene | 4932442K08Rik | (+) | | | | |
| argise type II | ARG2 | (+) | | | | |
| RIKEN cD D630002J15 gene | D630002J15Rik | (−) | | | | |
| ESTs | | (+) | | | | |
| papillary rel cell carcinoma | PRCC | (+) | (?) | RCC | conflict | |
| (translocation-associated) | | | | | | |
| growth differentiation factor 8 | GDF8 | (+) | | | | |
| thioredoxin 2 | TXN2 | (−) | | | | |
| renin 2 tandem duplication of Ren1 | Ren2 | (−) | | | | |
| Unknown | | (+) | | | | |
| calbindin-28K | CALB1 | (−) | (−) | RCC | C | |
| secreted acidic cysteine rich | SPARC | (+) | (+) | RCC | C | |
| glycoprotein | | | | | | |
| calcium channel, voltage-dependent, | CACNB3 | (+) | (+) | RCC | C | |
| beta 3 subunit | | | | | | |
| expressed sequence AI604920 | KIAA0297 | (+) | | | | |
| | KIAA0329 | | | | | |
| RIKEN cD 5133401H06 gene | 5133401H06Rik | (−) | | | | |
| expressed sequence AI314027 | GLS | (+) | | | | |
| PPAR gamma coactivator-1beta | PERC | (−) | | | | |
| protein | | | | | | |
| chaperonin subunit 3 (gamma) | CCT3 | (+) | | | | |
| coproporphyrinogen oxidase | CPO | (−) | | | | |
| erythroid differentiation regulator | edr | (+) | | | | |
| polymerase, gamma | POLG | (−) | | | | |
| cathepsin S | CTSS | (+) | (+) | RCC | C | |
| expressed sequence AI844876 | AI844876 | (−) | | | | |
| RIKEN cD 3010001A07 gene | BFAR | (−) | | | | |
| expressed sequence AI586180 | AI586180 | (+) | | | | |
| tetratricopeptide repeat domain | TTC3 | (+) | (+) | RCC | C | |
| Mus musculus, clone MGC: 6377 | ME2 | (+) | | | | |
| IMAGE: 3499365, mR, complete cds | | | | | | |
| smoothelin | SMTN | (+) | | | | |
| complement component 1, q | C1QA | (+) | (+) | RCC | C | |
| subcomponent, alpha polypeptide | | | | | | |
| Unknown | | (−) | | | | |
| glycerol phosphate dehydrogese 1, | GPD2 | (−) | | | | |
| mitochondrial | | | | | | |
| ribosomal protein S26 | RPS26 | (+) | | | | |
| protein tyrosine phosphatase, receptor | PTPRB | (−) | (+) | RCC | DC | |
| type, B | | | | | | |
| expressed sequence AW493404 | AW493404 | (+) | | | | |
| RIKEN cD 4930506M07 gene | FLJ11122 | (+) | | | | |
| solute carrier family 35, member A5; | SLC35A5 | (−) | | | | |
| RIKEN cD 1010001J06 gene | | | | | | |
| Mus musculus, clone MGC: 36388 | MCSC | (−) | | | | |
| IMAGE: 5098924, mR, complete cds | | | | | | |
| coagulation factor III | F3 | (+) | | | | |
| ESTs, Weakly similar to ADT1 | SLC25A16 | (−) | | | | |
| MOUSE ADP, ATP CARRIER | | | | | | |
| PROTEIN, HEART/SKELETAL | | | | | | |
| MUSCLE ISOFORM T1 | | | | | | |
| (M. musculus) | | | | | | |
| expressed sequence AI449309 | AI449309 | (+) | | | | |
| max binding protein | MNT | (+) | | | | |
| fatty acid synthase | FASN | (−) | | | | (+) |
| hypothetical protein, MGC: 6957 | MGC6957 | (+) | | | | |
| (2610524K04Rik; RIKEN cD | pp90RSK4 | (+) | | | | |
| 2610524K04 gene) | | | | | | |
| expressed sequence AW045860 | AW045860 | (−) | | | | |
| ESTs | | (−) | | | | |
| ribosomal protein L7 | RPL7 | (+) | (+) | RCC | C | |
| solute carrier family 34 (sodium | SLC34A2 | (+) | | | | |
| phosphate), member 2 | | | | | | |
| fumarylacetoacetate hydrolase | FAH | (−) | (−) | RCC | C | |
| Mus musculus, Similar to ribosomal | | (+) | | | | |
| protein S20, clone MGC: 6876 | | | | | | |
| IMAGE: 2651405, mR, complete cds | | | | | | |
| single Ig IL-1 receptor related protein | SIGIRR | (−) | (−) | RCC | C | |
| expressed sequence AI528491 | AI528491 | (−) | | | | |
| RIKEN cD 2810468K17 gene | MGC13272 | (+) | | | | |
| ESTs | | (−) | | | | |
| mitogen-activated protein kise 7 | MAPK7 | (+) | | | | (+) |
| Mus musculus, clone MGC: 19361 | | (+) | | | | |
| IMAGE: 4242170, mR, complete cds | | | | | | |
| schlafen 4 | FLJ10260 | (+) | | | | |
| RIKEN cD 1810036E22 gene | | (−) | | | | |
| flotillin 2 | FLOT2 | (+) | | | | |
| nicotimide nucleotide transhydrogese | NNT | (−) | (−) | RCC | C | |
| expressed sequence AI661919 | AI661919 | (−) | | | | |
| deoxyribonuclease I | DNASE1 | (−) | | | | |
| Mus musculus, Similar to ubiquitin- | UBE2V1 | (−) | (+) | RCC | DC | |
| conjugating enzyme E2 variant 1, | | | | | | |
| clone MGC: 7660 IMAGE: 3496088, | | | | | | |
| mR, complete cds | | | | | | |
| Mus musculus, clone | DLAT | (−) | | | | |
| IMAGE: 3586777, mR, partial cds | | | | | | |
| RIKEN cD 1200015A22 gene | MGC3222 | (+) | | | | |
| RIKEN cD 5830445O15 gene | 5830445O15Rik | (−) | | | | |
| 2-hydroxyphytanoyl-CoA lyase | HPCL2 | (−) | (−) | RCC | C | |
| serine (or cysteine) proteise inhibitor, | SERPING1 | (+) | (+) | RCC | C | |
| clade G (C1 inhibitor), member 1 | | | | | | |
| FK506 binding protein 10 (65 kDa) | FKBP10 | (+) | | | | |
| calsyntenin 1 | CLSTN1 | (−) | (−) | RCC | C | |
| RIKEN cD 2600001N01 gene | ZWINT | (+) | | | | |
| adenylosuccite synthetase 2, non | ADSS | (+) | | | | |
| muscle | | | | | | |
| cryptochrome 2 (photolyase-like) | CRY2 | (−) | | | | |
| solute carrier family 12, member 1 | SLC12A1 | (−) | (−) | RCC | C | (+) |
| S100 calcium binding protein A4 | S100A4 | (+) | | | | |
| E74-like factor 3 | ELF3 | (+) | (+) | RCC | C | |
| RIKEN cD 2900074L19 gene | | (−) | | | | |
| laminin, alpha 2 | LAMA2 | (+) | (+) | RCC | C | |
| solute carrier family 25 | SLC25A10 | (−) | | | | |
| (mitochondrial carrier | | | | | | |
| Mus musculus, clone MGC: 18871 | GLYAT | (−) | (−) | RCC | C | |
| IMAGE: 4234793, mR, complete cds | | | | | | |
| macrophage expressed gene 1 | MPEG1 | (+) | | | | |
| RIKEN cD 2810430J06 gene | FRCP1 | (+) | | | | |
| expressed sequence AW552393 | AW552393 | (−) | | | | |
| cofilin 1, non-muscle | CFL1 | (+) | (+)/(−) | RCC | conflict | |
| expressed sequence AI875199 | AI875199 | (−) | | | | |
| expressed sequence BB120430 | BB120430 | (+) | | | | |
| ESTs, Weakly similar to B Chain B, | | (+) | | | | |
| Crystal Structure Of Murine Soluble | | | | | | |
| Epoxide Hydrolase Complexed With | | | | | | |
| Cdu Inhibitor (M. musculus) | | | | | | |
| ESTs, Weakly similar to DRR1 | | (−) | | | | |
| (H. sapiens) | | | | | | |
| Mus musculus, Similar to KIAA0763 | KIAA0763 | (−) | | | | |
| gene product, clone | | | | | | |
| IMAGE: 4503056, mR, partial cds | | | | | | |
| expressed sequence AI875557 | AI875557 | (−) | | | | |
| expressed sequence AI848669 | AI848669 | (−) | | | | |
| RIKEN cD 2610305D13 gene | FLJ11191 | (+) | | | | |
| liver-specific bHLH-Zip transcription | Lisch7 | (+) | | | | (+) |
| factor | | | | | | |
| phosphodiesterase 1A, calmodulin- | PDE1A | (−) | (−) | RCC | C | |
| dependent | | | | | | |
| ATP synthase, H+ transporting, | ATP5A1 | (−) | | | | |
| mitochondrial F1 complex, alpha | | | | | | |
| subunit, isoform 1 | | | | | | |
| laminin receptor 1 (67 kD, ribosomal | LAMR1 | (+) | (+) | RCC | C | |
| protein SA) | | | | | | |
| ESTs | | (−) | | | | |
| runt related transcription factor 1 | RUNX1 | (+) | | | | |
| leukotriene C4 synthase | LTC4S | (+) | | | | |
| RIKEN cD 9130022E05 gene | 9130022E05Rik | (−) | | | | |
| methyl CpG binding protein 2 | MECP2 | (−) | | | | |
| expressed sequence AI835705 | AI835705 | (−) | | | | |
| a disintegrin and metalloproteise | ADAM12 | (+) | | | | |
| domain 12 (meltrin alpha) | | | | | | |
| Mus musculus chemokine receptor | CCRL1 | (−) | | | | |
| CCX CKR mR, complete cds, | | | | | | |
| altertively spliced | | | | | | |
| AXL receptor tyrosine kise | AXL | (+) | | | | |
| aldo-keto reductase family 1, member | Akr1c18 | (−) | | | | |
| C18; expressed sequence AW146047 | | | | | | |
| protein tyrosine phosphatase, receptor | PTPRCAP | (+) | | | | |
| type, C polypeptide-associated | | | | | | |
| protein | | | | | | |
| kinesin family member 21A | KIF21A | (−) | (+) | RCC | DC | |
| Kruppel-like factor 15 | KLF15 | (−) | | | | |
| RIKEN cD 2610039E05 gene | 2610039E05Rik | (−) | | | | |
| platelet derived growth factor | PDGFRB | (+) | | | | |
| receptor, beta polypeptide | | | | | | |
| expressed sequence AI413466 | PPP1R1B | (−) | | | | |
| thrombospondin 1 | THBS1 | (+) | (−) | RCC | DC | |
| TRAF-interacting protein | TRIP | (+) | | | | |
| RIKEN cD 2700099C19 gene | LOC51248 | (+) | | | | |
| SH3 domain protein 3 | OSTF1 | (+) | | | | |
| 5′,3′ nucleotidase, cytosolic | NT5C | (+) | | | | |
| RIKEN cD 1700028A24 gene | LOC55862 | (−) | | | | |
| expressed sequence AW743884 | AW743884 | (+) | | | | |
| epidermal growth factor-containing | EFEMP2 | (+) | | | | |
| fibulin-like extracellular matrix | | | | | | |
| protein 2 | | | | | | |
| Mus musculus adult male liver cD, | CSAD | (−) | | | | |
| RIKEN full-length enriched library, | | | | | | |
| clone:1300015E02:deoxyribonuclease | | | | | | |
| II alpha, full insert sequence | | | | | | |
| RIKEN cD 2010315L10 gene | MDS032 | (+) | | | | |
| ribosomal protein L18 | RPL18 | (+) | (+) | RCC | C | |
| microfibrillar associated protein 5 | MGP2 | (+) | | | | |
| aldehyde dehydrogese family 1, | ALDH1A2 | (+) | | | | |
| subfamily A2 | | | | | | |
| adenylate kise 4 | Ak4 | (−) | | | | |
| E74-like factor 4 (ets domain | ELF4 | (+) | | | | |
| transcription factor) | | | | | | |
| G protein-coupled receptor kise 7 | MKNK2 | (−) | (+) | RCC | DC | |
| forkhead box M1 | FOXM1 | (+) | | | | |
| solute carrier family 22 (organic | SLC22A4 | (−) | | | | |
| cation transporter), member 4 | | | | | | |
| claudin 7 | CLDN7 | (+) | | | | |
| proteasome (prosome, macropain) | PSMB1 | (+) | | | | |
| subunit, beta type 1 | | | | | | |
| solute carrier family 22 (organic | SLC22A5 | (−) | | | | |
| cation transporter), member 5 | | | | | | |
| UDP-glucuronosyltransferase 1 | UGT1A@ | (−) | | | | |
| family, member 1 | | | | | | |
| glutathione S-transferase, pi 2 | Gstp2 | (+) | | | | |
| ESTs | | (−) | | | | |
| cystatin C | CST3 | (+) | | | | |
| transcription factor 4 | TCF4 | (+) | | | | |
| RIKEN cD 2610301D06 gene | 2610301D06Rik | (+) | | | | |
| tyrosine 3-monooxygese/tryptophan | YWHAE | (+) | | | | |
| 5-monooxygese activation protein, | | | | | | |
| epsilon polypeptide | | | | | | |
| methylmalonyl-Coenzyme A mutase | MUT | (−) | | | | (+) |
| myosin light chain, alkali, cardiac | MYL4 | (+) | | | | |
| atria | | | | | | |
| enhancer of zeste homolog 2 | EZH2 | (+) | | | | |
| (Drosophila) | | | | | | |
| RIKEN cD 0610025G13 gene | RPL38 | (+) | (−)/(+) | RCC | conflict | |
| Unknown | COL18A1 | (+) | | | | |
| Tial1 cytotoxic granule-associated R | TIAL1 | (+) | (+) | RCC | C | |
| binding protein-like 1 | | | | | | |
| ribosomal protein S14 | RPS14 | (+) | (+) | RCC | C | |
| numb gene homolog (Drosophila) | NUMB | (+) | | | | |
| RIKEN cD 1300004O04 gene | CACH-1 | (−) | | | | |
| adducin 3 (gamma) | ADD3 | (−) | (+) | RCC | DC | (+) |
| vitamin D receptor | VDR | (−) | | | | |
| ribosomal protein L5 | RPL5 | (+) | | | | |
| RIKEN cD 1810023B24 gene | FLJ14503 | (+) | | | | |
| RIKEN cD 3010027G13 gene | DKFZp434C119.1 | (−) | | | | |
| high mobility group AT-hook 1 | HMGA1 | (+) | | | | |
| endonuclease G | ENDOG | (−) | | | | |
| septin 8 | KIAA0202 | (+) | | | | |
| double cortin and | DCAMKL1 | (+) | | | | |
| calcium/calmodulin-dependent | | | | | | |
| protein kise-like 1 | | | | | | |
| procollagen, type I, alpha 2 | COL1A2 | (+) | (+) | RCC | C | |
| Mus musculus, hypothetical protein | RPS6KL1 | (−) | | | | |
| MGC11287 similar to ribosomal | | | | | | |
| protein S6 kise,, clone MGC: 28043 | | | | | | |
| IMAGE: 3672127, mR, complete cds | | | | | | |
| kallikrein 6 | Klk1 | (−) | (+) | RCC | DC | |
| mini chromosome maintence | MCM3 | (+) | (+) | RCC | C | |
| deficient (S. cerevisiae) | | | | | | |
| cartilage oligomeric matrix protein | COMP | (−) | | | | |
| pantophysin | HLF | (−) | | | | |
| macrophage scavenger receptor 2 | Msr2 | (+) | | | | |
| ESTs, Weakly similar to S65210 | | (−) | | | | |
| hypothetical protein YPL191c - yeast | | | | | | |
| (Saccharomyces cerevisiae) | | | | | | |
| (S. cerevisiae) | | | | | | |
| expressed sequence AI593249 | AI593249 | (−) | | | | |
| tumor rejection antigen gp96 | TRA1 | (+) | (+) | RCC | C | (+) |
| Unknown | | (+) | | | | |
| lysozyme | LYZ | (+) | (+) | RCC | C | |
| ATPase, +/K+ transporting, beta 1 | ATP1B1 | (−) | (+) | RCC | DC | (+) |
| polypeptide | | | | | | |
| lysosomal-associated protein | LAPTM5 | (+) | (+) | RCC | C | |
| transmembrane 5 | | | | | | |
| Yamaguchi sarcoma viral (v-yes) | YES1 | (+) | | | | |
| oncogene homolog | | | | | | |
| gamma-glutamyl transpeptidase | GGT1 | (−) | | | | |
| chitise 3-like 3 | CHIA | (+) | | | | |
| ESTs, Weakly similar to JE0096 | | (+) | | | | |
| myocilin - mouse (M. musculus) | | | | | | |
| peptidylprolyl isomerase C | PPIC | (−) | | | | |
| solute carrier family 7 (cationic | SLC7A9 | (−) | | | | |
| amino acid transporter, y+ system), | | | | | | |
| member 9 | | | | | | |
| fibrillarin | FBL | (+) | (+) | RCC | C | |
| RIKEN cD 2610029K21 gene | FLJ20249 | (+) | | | | |
| mutS homolog 2 (E. coli) | MSH2 | (+) | (+) | RCC | C | |
| TYRO protein tyrosine kise binding | TYROBP | (+) | (+) | RCC | C | |
| protein | | | | | | |
| RIKEN cD 6430559E15 gene | HT036 | (−) | | | | |
| ESTs | 1110069O07Rik | (−) | | | | |
| ras homolog gene family, member E | ARHE | (−) | (+) | RCC | DC | |
| stromal cell derived factor 1 | CXCL12 | (−) | | | | |
| cadherin 3 | CDH3 | (+) | | | | |
| small inducible cytokine B subfamily, | SCYB6 | (+) | | | | |
| member 5 | | | | | | |
| heparin binding epidermal growth | DTR | (+) | | | | |
| factor-like growth factor | | | | | | |
| AE binding protein 1 | AEBP1 | (+) | | | | |
| poliovirus receptor-related 3 | PVRL3 | (+) | (+) | RCC | C | |
| ESTs | | (+) | | | | |
| phospholipase A2, group IIA | PLA2G2A | (−) | | | | |
| (platelets, synovial fluid) | | | | | | |
| guanine nucleotide binding protein (G | GNG2 | (+) | | | | |
| protein), gamma 2 subunit | | | | | | |
| nidogen 1 | NID | (+) | (+) | RCC | C | |
| integrin beta 1 (fibronectin receptor | ITGB1 | (+) | (+) | RCC | C | |
| beta) | | | | | | |
| protein tyrosine phosphatase, receptor | PTPRO | (+) | (−) | RCC | DC | |
| type, O | | | | | | |
| retinoic acid induced 1 | RAI1 | (+) | | | | |
| cell division cycle 2 homolog A | CDC2 | (+) | | | | |
| (S. pombe) | | | | | | |
| homeo box B7 | HOXB7 | (+) | | | | |
| matrix metalloproteise 7 | MMP7 | (+) | (+) | RCC | C | |
| Kruppel-like factor 1 (erythroid) | KLF1 | (−) | | | | |
| ESTs | | (−) | | | | |
| feline sarcoma oncogene | FES | (+) | (+) | RCC | C | |
| reticulocalbin | RCN1 | (+) | (+) | RCC | C | |
| aconitase 1 | ACO1 | (−) | (−) | RCC | C | |
| CCCTC-binding factor | CTCF | (+) | | | | |
| integrin alpha M | ITGAM | (+) | (+) | RCC | C | |
| serine (or cysteine) proteise inhibitor, | SERPINB2 | (+) | | | | |
| clade B (ovalbumin), member 2 | | | | | | |
| solute carrier family 16 | SLC16A2 | (−) | (−) | RCC | C | |
| (monocarboxylic acid transporters), | | | | | | |
| member 2 | | | | | | |
| Hoxc8 | MCM5 | (+) | | | | |
| Mus musculus, Similar to | | (−) | | | | |
| angiopoietin-like factor, clone | | | | | | |
| MGC: 32448 IMAGE: 5043159, mR, | | | | | | |
| complete cds | | | | | | |
| ESTs | | (−) | | | | |
| ring finger protein (C3HC4 type) 19 | RNF19 | (+) | | | | (+) |
| ESTs, Weakly similar to | | (−) | | | | |
| TYROSINE-PROTEIN KISE JAK3 | | | | | | |
| (M. musculus) | | | | | | |
| eukaryotic translation initiation factor | EIF4G2 | (+) | (+) | RCC | C | |
| 4, gamma 2 | | | | | | |
| ribosomal protein S7 | RPS7 | (+) | | | | |
| acidic ribosomal phosphoprotein PO | RPLP0 | (+) | (+) | RCC | C | (+) |
| ribosomal protein S5 | RPS5 | (+) | | | | |
| guanine nucleotide binding protein, | GNB2L1 | (+) | (+) | RCC | C | |
| beta 2, related sequence 1 | | | | | | |
| meprin 1 alpha | MEP1A | (−) | (+) | RCC | DC | |
| aldo-keto reductase family 1, member | AKR1B10 | (+) | | | | |
| B8 ((Fgfrp) fibroblast growth factor | | | | | | |
| regulated protein) | | | | | | |
| phosphoprotein enriched in astrocytes | PEA15 | (+) | (+) | RCC | C | (+) |
| 15 | | | | | | |
| RIKEN cD 2600017H24 gene | | (+) | | | | |
| cytochrome c oxidase, subunit VIc | COX6C | (−) | (+) | RCC | DC | |
| interferon gamma receptor | IFNGR1 | (+) | (+) | RCC | C | (+) |
| ADP-ribosyltransferase (D+ | ADPRTL2 | (+) | | | | |
| D-dopachrome tautomerase | DDT | (−) | (−) | RCC | C | |
| annexin A2 | ANXA2 | (+) | (−)/(+) | RCC | conflict | |
| expressed sequence AI852479 | CDKL3 | (−) | | | | |
| ribosomal protein L6 | RPL6 | (+) | (+) | RCC | C | |
| solute carrier family 22 (organic | SLC22A1 | (−) | (+) | RCC | DC | |
| cation transporter), member 1 | | | | | | |
| platelet-activating factor | PAFAH1B3 | (+) | | | | |
| acetylhydrolase, isoform 1b, alpha1 | | | | | | |
| subunit | | | | | | |
| inosine 5′-phosphate dehydrogese 2 | IMPDH2 | (+) | | | | |
| clathrin, light polypeptide (Lca) | CLTA | (+) | | | | |
| cystatin B | CSTB | (+) | | | | |
| pre B-cell leukemia transcription | PBX1 | (−) | | | | |
| factor 1 | | | | | | |
| annexin A4 | ANXA4 | (+) | (+) | RCC | C | (+) |
| small proline-rich protein 1A | SPRR1A | (+) | | | | |
| chemokine (C-C) receptor 2 | CCR2 | (+) | (+) | RCC | C | |
| nucleophosmin 1 | NPM1 | (+) | (+) | RCC | C | |
| solute carrier family 15 (H+/peptide | SLC15A2 | (−) | | | | |
| transporter), member 2 | | | | | | |
| CD24a antigen | CD24 | (+) | (+) | RCC | C | |
| ribosomal protein S15 | RPS15 | (+) | | | | |
| ribosomal protein S15 | SYN1 | (+) | | | | |
| Mus musculus, clone MGC: 36997 | MGC36997 | (+) | | | | |
| IMAGE: 4948448, mR, complete cds | | | | | | |
| tropomyosin 2, beta | TPM2 | (+) | | | | |
| prion protein | PRNP | (−) | | | | |
| klotho | KL | (−) | (−) | RCC | C | |
| serine palmitoyltransferase, long | SPTLC1 | (−) | (+) | RCC | DC | |
| chain base subunit 1 | | | | | | |
| chemokine orphan receptor 1 | RDC1 | (+) | | | | |
| S100 calcium binding protein A13 | S100A13 | (+) | | | | |
| RIKEN cD 1500010B24 gene | E1F1A | (+) | (+) | RCC | C | (+) |
| calpain, small subunit 1 | CAPNS1 | (−) | (+) | RCC | DC | |
| Ngfi-A binding protein 2 | NAB2 | (+) | | | | |
| ribonucleotide reductase M1 | RRM1 | (−) | (+) | RCC | DC | |
| sulfotransferase-related protein | Sult-x1 | (+) | | | | |
| SULT-X1 | | | | | | |
| 4-hydroxyphenylpyruvic acid | HPD | (−) | (−) | RCC | C | |
| dioxygese | | | | | | |
| peroxiredoxin 5 | PRDX5 | (+) | (?) | RCC | conflict | |
| ribosomal protein S4, X-linked | RPS4X | (+) | | | | (+) |
| solute carrier family 27 (fatty acid | SLC27A2 | (−) | | | | |
| transporter), member 2 | | | | | | |
| isovaleryl coenzyme A dehydrogese | IVD | (−) | | | | |
| thymoma viral proto-oncogene 1 | AKT1 | (+) | (+) | RCC | C | |
| protein tyrosine phosphatase, non- | PTPN9 | (+) | | | | |
| receptor type 9 | | | | | | |
| SAR1a gene homolog (S. cerevisiae) | SAR1 | (+) | (−) | RCC | DC | |
| eukaryotic translation initiation factor | EIF4EBP1 | (+) | | | | |
| 4E binding protein 1 | | | | | | |
| RIKEN cD 4921537D05 gene | NY-REN-58 | (+) | | | | |
| transcription elongation regulator 1 | TCERG1 | (+) | | | | |
| (CA150) | | | | | | |
| keratin complex 2, basic, gene 8 | KRT8 | (+) | (+) | RCC | C | |
| ESTs, Weakly similar to JC7182 +- | SLC23A3 | (−) | | | | |
| dependent vitamin C (H. sapiens) | | | | | | |
| amine N-sulfotransferase | Sultn | (−) | | | | |
| ADP-ribosylation factor 1 | ARF1 | (+) | | | | |
| cyclin-dependent kise 4 | CDK4 | (+) | | | | (−) |
| ras homolog B (RhoB) | ARHB | (+) | (+) | RCC | C | |
| calbindin-D9K | CALB3 | (−) | | | | |
| baculoviral IAP repeat-containing 1a | BIRC1 | (+) | | | | |
| ESTs, Weakly similar to | C1QR1 | (+) | | | | |
| TYROSINE-PROTEIN KISE JAK3 | | | | | | |
| (M. musculus) | | | | | | |
| apoptosis inhibitory protein 5 | API5 | (+) | | | | |
| spectrin SH3 domain binding protein 1 | SSH3BP1 | (+) | | | | |
| ribosomal protein S3a | RPS3A | (+) | (+) | RCC | C | |
| calpain 2 | CAPN2 | (+) | | | | |
| ribosomal protein L12 | RPL12 | (+) | (+) | RCC | C | (+) |
| ribosomal protein S16 | RPS16 | (+) | (+) | RCC | C | |
| Ia-associated invariant chain | CD74 | (+) | (+) | RCC | C | |
| expressed sequence AI413331 | AI413331 | (+) | | | | |
| glucose regulated protein, 58 kDa | GRP58 | (+) | (+) | RCC | C | |
| amiloride binding protein 1 (amine | ABP1 | (+) | (+) | RCC | C | |
| oxidase, copper-containing) | | | | | | |
| ESTs, Weakly similar to YMP2- | 3230401L03Rik | (+) | | | | |
| CAEEL HYPOTHETICAL 30.3 KD | | | | | | |
| PROTEIN B0361.2 IN | | | | | | |
| CHROMOSOME III (C. elegans) | | | | | | |
| annexin A3 | ANXA3 | (+) | | | | |
| dolichyl-di-phosphooligosaccharide- | DDOST | (+) | | | | |
| protein glycotransferase | | | | | | |
| anterior gradient 2 (Xenopus laevis) | AGR2 | (−) | | | | |
| T-box 6 | TBX6 | (+) | | | | |
| procollagen, type V, alpha 1 | COL5A1 | (+) | (+) | RCC | C | (+) |
| D segment, Chr 17, human D6S56E 2 | LSM2 | (+) | | | | |
| cellular nucleic acid binding protein | ZNF9 | (+) | (+) | RCC | C | |
| claudin 4 | CLDN4 | (+) | | | | |
| fibrillin 1 | FBN1 | (+) | | | | |
| ubiquitin-like 1 | UBL1 | (+) | (+) | RCC | C | (+) |
| period homolog 1 (Drosophila) | PER1 | (−) | | | | |
| procollagen, type IV, alpha 1 | COL4A1 | (+) | (+) | RCC | C | |
| protein phosphatase 2a, catalytic | PPP2CB | (+) | (−) | RCC | DC | |
| subunit, beta isoform | | | | | | |
| Fas apoptotic inhibitory molecule | FAIM | (+) | | | | |
| ESTs | FLJ23447 | (−) | | | | |
| breakpoint cluster region protein 1 | BANF1 | (+) | | | | |
| RAN, member RAS oncogene family | RAN | (+) | (+) | RCC | C | |
| src-like adaptor protein | SLA | (+) | | | | (+) |
| A kise (PRKA) anchor protein 2 | AKAP2 | (+) | (−) | RCC | DC | |
| Unknown | | (−) | | | | |
| serine/threonine protein kise CISK | SGKL | (+) | | | | |
| D methyltransferase (cytosine-5) 1 | DNMT1 | (+) | | | | (+) |
| proteasome (prosome, macropain) | PSMB10 | (+) | (+) | RCC | C | (+) |
| subunit, beta type 10 | | | | | | |
| lymphocyte antigen 6 complex, locus E | LY6E | (+) | | | | |
| colony stimulating factor 1 | CSF1 | (+) | (+) | RCC | C | |
| (macrophage) | | | | | | |
| procollagen lysine, 2-oxoglutarate 5- | PLOD2 | (+) | (+) | RCC | C | (+) |
| dioxygese 2 | | | | | | |
| upstream transcription factor 1 | USF1 | (−) | | | | |
| ESTs, Moderately similar to T46312 | | (+) | | | | |
| hypothetical protein | | | | | | |
| DKFZp434J1111.1 (H. sapiens) | | | | | | |
| mago-shi homolog, proliferation- | MAGOH | (+) | (+) | RCC | C | |
| associated (Drosophila) | | | | | | |
| TG interacting factor | TGIF | (+) | (+) | RCC | C | (+) |
| lymphocyte antigen 6 complex, locus A | LY6H | (+) | | | | |
| non-catalytic region of tyrosine kise | NCK1 | (+) | (+) | RCC | C | |
| adaptor protein 1 | | | | | | |
| tissue inhibitor of metalloproteise | TIMP1 | (+) | (+) | RCC | C | (+) |
| proteasome (prosome, macropain) 28 | PSME1 | (+) | | | | |
| subunit, alpha | | | | | | |
| sigl sequence receptor, delta | SSR4 | (+) | (+) | RCC | C | |
| ESTs, Highly similar to organic | | (−) | | | | |
| cation transporter-like protein 2 | | | | | | |
| (M. musculus) | | | | | | |
| ESTs | | (−) | | | | |
| pyruvate kise liver and red blood cell | PKLR | (−) | (−) | RCC | C | |
| acyl-Coenzyme A oxidase 1, | ACOX1 | (−) | (+) | RCC | DC | |
| palmitoyl | | | | | | |
| CD59a antigen | CD59 | (−) | (+) | RCC | DC | (+) |
| period homolog 2 (Drosophila) | PER2 | (−) | | | | |
| peroxisomal sarcosine oxidase | PIPOX | (−) | (−) | RCC | C | |
| RIKEN cD 2810418N01 gene | KIAA0186 | (+) | | | | |
| 1-acylglycerol-3-phosphate O- | AGPAT3 | (−) | (−) | RCC | C | |
| acyltransferase 3; expressed | | | | | | |
| sequence AW493985 | | | | | | |
| ESTs | | (−) | | | | |
| cholinergic receptor, nicotinic, beta | CHRNB1 | (+) | | | | |
| polypeptide 1 (muscle) | | | | | | |
| ESTs | | (−) | | | | |
| adenylyl cyclase-associated CAP | CAP | (+) | | | | |
| protein homolog 1 | | | | | | |
| (S. cerevisiae, S. pombe) | | | | | | |
| thiamin pyrophosphokise | TPK1 | (−) | | | | |
| myocyte enhancer factor 2A | MEF2A | (+) | (+)/(−) | RCC | conflict | |
| ESTs, Weakly similar to limb | | (+) | | | | |
| expression 1 homolog (chicken) (Mus | | | | | | |
| musculus) (M. musculus) | | | | | | |
| toll-like receptor 2 | TLR2 | (+) | | | | |
| small inducible cytokine B subfamily | SCYB10 | (+) | | | | |
| (Cys-X-Cys), member 10 | | | | | | |
| ESTs | | (−) | | | | |
| glycerol-3-phosphate acyltransferase, | GPAT | (−) | | | | |
| mitochondrial | | | | | | |
| retinoic acid early transcript gamma | ULBP2 | (+) | | | | |
| mammary tumor integration site 6 | EIF3S6 | (+) | (+) | RCC | C | |
| CD72 antigen | CD72 | (+) | | | | |
| RAR-related orphan receptor alpha | RORA | (−) | | | | |
| testis derived transcript | TES | (+) | (+) | RCC | C | (+) |
| ESTs | | (+) | | | | |
| a disintegrin-like and metalloprotease | ADAMTS2 | (+) | | | | |
| (reprolysin type) with | | | | | | |
| thrombospondin type 1 motif, 2 | | | | | | |
| interleukin 1 receptor, type I | IL1R1 | (+) | | | | |
| ESTs | | (+) | | | | |
| D methyltransferase 3B | DNMT3B | (+) | | | | |
| RIKEN cD 2610524G09 gene | IER5 | (+) | | | | |
| Mus musculus, Similar to | FLJ20245 | (+) | | | | |
| hypothetical protein FLJ20245, clone | | | | | | |
| MGC: 7940 IMAGE: 3584061, mR, | | | | | | |
| complete cds | | | | | | |
| high mobility group nucleosomal | HMGN2 | (+) | (+) | RCC | C | |
| binding domain 2 | | | | | | |
| crystallin, mu | CRYM | (+) | (−) | RCC | DC | |
| H2A histone family, member Z | H2AFZ | (+) | (+) | RCC | C | |
| transcription factor Dp 1 | TFDP1 | (+) | (+) | RCC | C | |
| microtubule associated testis specific | MAST205 | (+) | | | | |
| serine/threonine protein kise | | | | | | |
| cathepsin L | CTSL | (+) | | | | (+) |
| kidney-derived aspartic protease-like | NAP1 | (−) | | | | |
| protein | | | | | | |
| interferon-induced protein with | IFIT3 | (+) | | | | |
| tetratricopeptide repeats 3 | | | | | | |
| sphingomyelin phosphodiesterase 2, | SMPD2 | (−) | | | | |
| neutral | | | | | | |
| growth arrest and D-damage- | GADD45G | (−) | (+) | RCC | DC | |
| inducible 45 gamma | | | | | | |
| vasodilator-stimulated | VASP | (+) | | | | |
| phosphoprotein | | | | | | |
| flavin containing monooxygese 1 | FMO1 | (−) | (−) | RCC | C | |
| CD38 antigen | CD38 | (+) | | | | |
| tescin C | TNC | (+) | | | | |

TABLE 10

| | | | | Number | Average | Number |
|---|---|---|---|---|---|---|
| | Ontology | Average | Average | Genes | Expression | Genes |
| | Category | Expression | Expression UP | UP | DOWN | DOWN |
| | | Early (A) | | | | |
| Early (A) | oxidative | −0.418 | 0 | 0 | −1.67 | 4 |
| | phosphorylation | | | | | |
| | DNA replication | 0.692 | 3.46 | 5 | 0 | 0 |
| | initiation | | | | | |
| | DNA dependent DNA | 0.461 | 4.86 | 9 | −0.25 | 1 |
| | replication | | | | | |
| | regulation of translation | 0.003 | 1.33 | 4 | −1.31 | 3 |
| | group transfer | −0.452 | 0 | 0 | −2.26 | 5 |
| | coenzyme metabolism | | | | | |
| | ribonucleoside | −0.256 | 0.41 | 1 | −1.69 | 4 |
| | triphosphate | | | | | |
| | biosynthesis | | | | | |
| | purine nucleoside | −0.256 | 0.41 | 1 | −1.69 | 4 |
| | triphosphate | | | | | |
| | biosynthesis | | | | | |
| | purine ribonucleoside | −0.256 | 0.41 | 1 | −1.69 | 4 |
| | triphosphate | | | | | |
| | biosynthesis | | | | | |
| | glycolysis | −0.163 | 0.85 | 2 | −2.15 | 6 |
| | nucleoside triphosphate | −0.112 | 1.02 | 2 | −1.69 | 4 |
| | metabolism | | | | | |
| | glucose metabolism | −0.347 | 0.85 | 2 | −5.01 | 10 |
| | hexose catabolism | −0.163 | 0.85 | 2 | −2.15 | 6 |
| | glucose catabolism | −0.163 | 0.85 | 2 | −2.15 | 6 |
| | alcohol catabolism | −0.163 | 0.85 | 2 | −2.15 | 6 |
| | moNumbersaccharide | −0.163 | 0.85 | 2 | −2.15 | 6 |
| | catabolism | | | | | |
| | moNumbersaccharide | −0.376 | 0.85 | 2 | −5.74 | 11 |
| | metabolism | | | | | |
| | purine ribonucleotide | −0.108 | 1.04 | 2 | −1.69 | 4 |
| | biosynthesis | | | | | |
| | hexose metabolism | −0.347 | 0.85 | 2 | −5.01 | 10 |
| | carbohydrate catabolism | −0.163 | 0.85 | 2 | −2.15 | 6 |
| | S phase of mitotic cell | 0.389 | 6.14 | 12 | −0.7 | 2 |
| | cycle | | | | | |
| | DNA replication | 0.389 | 6.14 | 12 | −0.7 | 2 |
| | main pathways of | −0.225 | 0.85 | 2 | −3.1 | 8 |
| | carbohydrate | | | | | |
| | metabolism | | | | | |
| | energy derivation by | −0.310 | 1.41 | 3 | −5.44 | 10 |
| | oxidation of organic | | | | | |
| | compounds | | | | | |
| | DNA replication and | 0.382 | 6.43 | 13 | −0.7 | 2 |
| | chromosome cycle | | | | | |
| | energy pathways | −0.353 | 1.41 | 3 | −6.71 | 12 |
| | mitotic cell cycle | 0.459 | 13.32 | 24 | −0.93 | 3 |
| | alcohol metabolism | −0.341 | 1.19 | 3 | −6.65 | 13 |
| | DNA metabolism | 0.388 | 16.14 | 31 | −2.19 | 5 |
| | carbohydrate | −0.256 | 3.12 | 8 | −9.27 | 16 |
| | metabolism | | | | | |
| | cell cycle | 0.437 | 19.95 | 39 | −1.15 | 4 |
| | cell proliferation | 0.391 | 26.07 | 49 | −3.79 | 8 |
| | cell growth and/or | 0.108 | 49.42 | 96 | −32.32 | 62 |
| | maintenance | | | | | |
| | metabolism | 0.092 | 73.79 | 156 | −50.72 | 94 |
| | proton-transporting two- | −0.423 | 0 | 0 | −1.69 | 4 |
| | sector ATPase complex | | | | | |
| | hydrogen-translocating | −0.423 | 0 | 0 | −1.69 | 4 |
| | F-type ATPase complex | | | | | |
| | inner membrane | −0.387 | 0.64 | 2 | −5.67 | 11 |
| | mitochondrial inner | −0.371 | 0.64 | 2 | −4.72 | 9 |
| | membrane | | | | | |
| | extrachromosomal DNA | −0.194 | 1.97 | 5 | −4.49 | 8 |
| | extrachromosomal | −0.194 | 1.97 | 5 | −4.49 | 8 |
| | circular DNA | | | | | |
| | cytoplasm | 0.059 | 56.82 | 118 | −44.87 | 84 |
| | intracellular | 0.110 | 85.21 | 179 | −54.11 | 105 |
| | ATP-binding and | −0.477 | 0 | 0 | −1.43 | 3 |
| | phosphorylation- | | | | | |
| | dependent chloride | | | | | |
| | channel activity | | | | | |
| | intramolecular | −0.724 | 0 | 0 | −3.62 | 5 |
| | isomerase activity\, | | | | | |
| | transposing C═C bonds | | | | | |
| | cyclophilin-type | 0.336 | 1.9 | 4 | −0.22 | 1 |
| | peptidy-prolyl cis-trans | | | | | |
| | isomerase activity | | | | | |
| | cis-trans isomerase | 0.170 | 1.9 | 4 | −0.88 | 2 |
| | activity | | | | | |
| | peptidyl-prolyl cis-trans | 0.336 | 1.9 | 4 | −0.22 | 1 |
| | isomerase activity | | | | | |
| | intramolecular | −0.533 | 0.42 | 1 | −3.62 | 5 |
| | isomerase activity | | | | | |
| | growth factor binding | −0.453 | 0.38 | 1 | −3.1 | 5 |
| | transferase activity\, | 0.031 | 2 | 4 | −1.78 | 3 |
| | transferring alkyl or aryl | | | | | |
| | (other than methyl) | | | | | |
| | groups | | | | | |
| | lyase activity | −0.218 | 2.48 | 5 | −5.75 | 10 |
| | isomerase activity | −0.217 | 2.32 | 5 | −5.57 | 10 |
| | hydrogen ion transporter | −0.441 | 0 | 0 | −4.41 | 10 |
| | activity | | | | | |
| | magnesium ion binding | −0.199 | 1.06 | 2 | −3.05 | 8 |
| | moNumbervalent | −0.441 | 0 | 0 | −4.41 | 10 |
| | iNumberrganic cation | | | | | |
| | transporter activity | | | | | |
| | carrier activity | −0.326 | 3.6 | 7 | −12.73 | 21 |
| | catalytic activity | 0.017 | 51.13 | 112 | −47.73 | 92 |
| | fatty acid metabolism | −0.550 | 0.74 | 2 | −6.24 | 8 |
| Early (A) | carboxylic acid | −0.524 | 1.36 | 4 | −12.37 | 17 |
| and again | metabolism | | | | | |
| in Early | organic acid metabolism | −0.524 | 1.36 | 4 | −12.37 | 17 |
| & Late (*) | biosynthesis | 0.051 | 15.77 | 30 | −13.07 | 23 |
| | physiological processes | 0.099 | 108.2 | 218 | −73.12 | 138 |
| | mitochondrion | −0.393 | 2.98 | 8 | −19.88 | 35 |
| | cytosol | 0.340 | 10.55 | 21 | −2.05 | 4 |
| | oxidoreductase activity | −0.377 | 4.45 | 9 | −17.66 | 26 |
| | | Late (B) | | | | |
| Late (B) | urea cycle intermediate | 0.243 | 1.13 | 2 | −0.4 | 1 |
| | metabolism | | | | | |
| | antigen presentation\, | 0.767 | 2.3 | 3 | 0 | 0 |
| | endogeNumberus antigen | | | | | |
| | antigen processing\, | 0.767 | 2.3 | 3 | 0 | 0 |
| | endogeNumberus antigen via | | | | | |
| | MHC class I | | | | | |
| | antigen presentation | 1.123 | 6.74 | 6 | 0 | 0 |
| | antigen processing | 1.123 | 6.74 | 6 | 0 | 0 |
| | immune response | 0.842 | 24.77 | 24 | −2.03 | 3 |
| | response to wounding | 0.384 | 5.53 | 8 | −1.69 | 2 |
| | response to | 0.791 | 13.56 | 13 | −1.69 | 2 |
| | pest/pathogen/parasite | | | | | |
| | catabolism | 0.526 | 16.21 | 25 | −1.48 | 3 |
| | proteasome core complex | 0.595 | 2.38 | 4 | 0 | 0 |
| | (sensu Eukarya) | | | | | |
| | microfibril | 1.296 | 9.07 | 7 | 0 | 0 |
| | extracellular matrix | 0.963 | 17.34 | 18 | 0 | 0 |
| | MHC class I receptor activity | 0.767 | 2.3 | 3 | 0 | 0 |
| | collagenase activity | 0.877 | 2.63 | 3 | 0 | 0 |
| | phospholipase inhibitor activity | 0.897 | 2.69 | 3 | 0 | 0 |
| | hydrolase activity\, acting on | 0.517 | 1.55 | 3 | 0 | 0 |
| | carbon-nitrogen (but Numbert | | | | | |
| | peptide) bonds\, in linear | | | | | |
| | amidines | | | | | |
| | apoptosis inhibitor activity | 0.486 | 2.43 | 5 | 0 | 0 |
| | hydrolase activity\, acting on | 0.483 | 2.9 | 6 | 0 | 0 |
| | carbon-nitrogen (but Numbert | | | | | |
| | peptide) bonds | | | | | |
| | transmembrane receptor | 0.622 | 16.24 | 21 | −1.31 | 3 |
| | activity | | | | | |
| | peptidase activity | 0.464 | 10.75 | 19 | −1.01 | 2 |
| | receptor activity | 0.513 | 20.32 | 30 | −2.36 | 5 |
| | signal transducer activity | 0.395 | 26.85 | 42 | −5.89 | 11 |
| Late (B) | defense response | 0.849 | 26.64 | 26 | −2.03 | 3 |
| and again in | response to biotic stimulus | 0.796 | 27.26 | 27 | −2.57 | 4 |
| Early & | response to external stimulus | 0.627 | 27.6 | 28 | −5.02 | 8 |
| Late (*) | extracellular space | 0.664 | 53.03 | 64 | −5.25 | 8 |
| | | Continues (*) | | | | |
| Late (B) | defense response | 0.696 | 16.7 | 24 | 0 | 0 |
| and again in | response to biotic stimulus | 0.523 | 16.7 | 24 | −2.57 | 3 |
| Early & | response to external stimulus | 0.438 | 20.77 | 29 | −5.02 | 7 |
| Late (*) | extracellular space | 0.247 | 39.54 | 49 | −21.77 | 23 |
| Early & | phenylalanine metabolism | −1.203 | 0 | 0 | −3.61 | 3 |
| Late (*) | phenylalanine catabolism | −1.203 | 0 | 0 | −3.61 | 3 |
| | aromatic amiNumber acid | −1.203 | 0 | 0 | −3.61 | 3 |
| | family catabolism | | | | | |
| | amiNumber acid catabolism | −1.036 | 0 | 0 | −5.18 | 5 |
| | amine catabolism | −1.036 | 0 | 0 | −5.18 | 5 |
| | amiNumber acid biosynthesis | −0.873 | 0 | 0 | −3.49 | 4 |
| | ribosome biogenesis | 0.872 | 8.72 | 10 | 0 | 0 |
| | ribosome biogenesis and | 0.872 | 8.72 | 10 | 0 | 0 |
| | assembly | | | | | |
| | iNumberrganic anion transport | 0.282 | 2.54 | 3 | −1.13 | 2 |
| | aromatic compound | −0.366 | 2.14 | 2 | −4.7 | 5 |
| | metabolism | | | | | |
| | posttranslational membrane | −0.049 | 2.62 | 4 | −2.96 | 3 |
| | targeting | | | | | |
| | blood coagulation | 0.340 | 3.86 | 5 | −1.48 | 2 |
| | anion transport | −0.034 | 2.54 | 3 | −2.78 | 4 |
| | hemostasis | 0.340 | 3.86 | 5 | −1.48 | 2 |
| | ER organization and biogenesis | −0.049 | 2.62 | 4 | −2.96 | 3 |
| | protein-ER targeting | −0.049 | 2.62 | 4 | −2.96 | 3 |
| | protein-membrane targeting | −0.049 | 2.62 | 4 | −2.96 | 3 |
| | amiNumber acid metabolism | −0.721 | 0.54 | 1 | −7.03 | 8 |
| | amiNumber acid and derivative | −0.782 | 0.54 | 1 | −9.14 | 10 |
| | metabolism | | | | | |
| | response to chemical substance | 0.564 | 6.12 | 8 | −1.04 | 1 |
| | amine metabolism | −0.782 | 0.54 | 1 | −9.14 | 10 |
| | response to abiotic stimulus | 0.435 | 8.97 | 11 | −2.45 | 4 |
| | cytoplasm organization and | 0.543 | 20.91 | 26 | −4.07 | 5 |
| | biogenesis | | | | | |
| | macromolecule biosynthesis | 0.771 | 16.2 | 21 | 0 | 0 |
| | protein biosynthesis | 0.771 | 16.2 | 21 | 0 | 0 |
| | cell organization and | 0.551 | 23.9 | 31 | −4.07 | 5 |
| | biogenesis | | | | | |
| | organelle organization and | 0.387 | 12.19 | 16 | −4.07 | 5 |
| | biogenesis | | | | | |
| | cytosolic ribosome (sensu | 0.823 | 9.87 | 12 | 0 | 0 |
| | Eukarya) | | | | | |
| | eukaryotic 48S initiation | 0.750 | 3 | 4 | 0 | 0 |
| | complex | | | | | |
| | cytosolic small ribosomal | 0.750 | 3 | 4 | 0 | 0 |
| | subunit (sensu Eukarya) | | | | | |
| | eukaryotic 43S pre-initiation | 0.688 | 3.44 | 5 | 0 | 0 |
| | complex | | | | | |
| | small ribosomal subunit | 0.746 | 3.73 | 5 | 0 | 0 |
| | actin filament | 0.340 | 2.02 | 3 | −0.66 | 1 |
| | ribosome | 0.786 | 16.5 | 21 | 0 | 0 |
| | ribonucleoprotein complex | 0.763 | 19.07 | 25 | 0 | 0 |
| | extracellular | 0.282 | 43.51 | 54 | −21.77 | 23 |
| | immuNumberglobulin binding | 1.103 | 3.31 | 3 | 0 | 0 |
| | anion transporter activity | −0.384 | 0.86 | 1 | −2.78 | 4 |
| | structural constituent of | 0.798 | 15.96 | 20 | 0 | 0 |
| | ribosome | | | | | |
| | chemokine activity | 0.902 | 4.51 | 5 | 0 | 0 |
| | G-protein-coupled receptor | 0.902 | 4.51 | 5 | 0 | 0 |
| | binding | | | | | |
| | chemokine receptor binding | 0.902 | 4.51 | 5 | 0 | 0 |
| | chemoattractant activity | 0.902 | 4.51 | 5 | 0 | 0 |
| | actin binding | 0.176 | 4.89 | 8 | −2.95 | 3 |
| | structural constituent of | 0.968 | 7.74 | 8 | 0 | 0 |
| | cytoskeleton | | | | | |
| | structural molecule activity | 0.842 | 32 | 38 | 0 | 0 |
| | ion transporter activity | −0.562 | 1.42 | 2 | −8.16 | 10 |
| | RNA binding | 0.605 | 13.09 | 17 | −1.59 | 2 |
| | Experiment Cons. | 70% up 30% | | | | |
| | | dn | | | | |

TABLE 11

| | | Size | | | | | | | |
|---|---|---|---|---|---|---|---|---|---|
| | | (Number | Concordance | | | | | | |
| | | of genes | | Average | Number | Average | Number | | |
| | | annotated | Average | Ex- | of | Ex- | of | | |
| Concordance/ | | to | Ex- | pression | Genes- | pression | Genes- | | |
| Disconcordance | Category | it by GO) | pression | UP | UP | Down | DOWN | EASE | Enrichment |
| Concordance | immuNumberglobulin binding | 6 | 1.103 | 3.31 | 3 | 0 | 0 | 0.034139907 | 9.728744939 |
| | selenium binding | 15 | −0.388 | 0.46 | 1 | −2.01 | 3 | 0.03816803 | 5.188663968 |
| | extracellular matrix structural | 19 | 0.886 | 4.43 | 5 | 0 | 0 | 0.014124581 | 5.120392073 |
| | constituent conferring tensile strength | | | | | | | | |
| | activity | | | | | | | | |
| | structural constituent of ribosome | 97 | 0.737 | 16.94 | 23 | 0 | 0 | 1.74394E−09 | 4.613631621 |
| | extracellular matrix structural | 39 | 0.802 | 4.81 | 6 | 0 | 0 | 0.046877828 | 2.993459981 |
| | constituent | | | | | | | | |
| | RNA binding | 207 | 0.563 | 16.21 | 27 | −0.44 | 1 | 4.8428E−06 | 2.631930998 |
| | structural molecule activity | 321 | 0.761 | 29.76 | 37 | −0.85 | 1 | 1.64291E−06 | 2.303378864 |
| | cell adhesion molecule activity | 124 | 0.458 | 7.19 | 11 | −1.24 | 2 | 0.023941119 | 2.039898132 |
| | nucleic acid binding | 1059 | 0.502 | 36.8 | 64 | −2.68 | 4 | 0.028128757 | 1.249395006 |
| | cytosolic ribosome (sensu Eukarya) | 27 | 0.730 | 8.03 | 11 | 0 | 0 | 3.54196E−07 | 8.030034236 |
| | proteasome core complex (sensu | 14 | 0.563 | 2.25 | 4 | 0 | 0 | 0.030644703 | 5.631452581 |
| | Eukarya) | | | | | | | | |
| | eukaryotic 43S pre-initiation complex | 15 | 0.525 | 2.1 | 4 | 0 | 0 | 0.036912006 | 5.256022409 |
| | collagen | 20 | 0.886 | 4.43 | 5 | 0 | 0 | 0.016227565 | 4.927521008 |
| | small ribosomal subunit | 20 | 0.698 | 3.49 | 5 | 0 | 0 | 0.016227565 | 4.927521008 |
| | proteasome complex (sensu Eukarya) | 24 | 0.520 | 2.6 | 5 | 0 | 0 | 0.030406018 | 4.106267507 |
| | microfibril | 36 | 1.029 | 7.2 | 7 | 0 | 0 | 0.008478551 | 3.83251634 |
| | ribosome | 122 | 0.737 | 16.94 | 23 | 0 | 0 | 1.17058E−07 | 3.715835515 |
| | basement membrane | 27 | 0.804 | 4.02 | 5 | 0 | 0 | 0.044662498 | 3.650015562 |
| | ribonucleoprotein complex | 186 | 0.701 | 20.34 | 29 | 0 | 0 | 1.18392E−07 | 3.073077618 |
| | cytosol | 193 | 0.601 | 14.42 | 21 | −0.59 | 2 | 0.000240127 | 2.348870118 |
| | extracellular matrix | 156 | 0.873 | 14.36 | 15 | −0.39 | 1 | 0.0116109 | 2.02154708 |
| | phenylalanine metabolism | 4 | −1.203 | 0 | 0 | −3.61 | 3 | 0.014752454 | 14.52356557 |
| | phenylalanine catabolism | 4 | −1.203 | 0 | 0 | −3.61 | 3 | 0.014752454 | 14.52356557 |
| | tyrosine metabolism | 5 | −1.033 | 0 | 0 | −3.1 | 3 | 0.02375814 | 11.61885246 |
| | aromatic amiNumber acid family | 5 | −1.203 | 0 | 0 | −3.61 | 3 | 0.02375814 | 11.61885246 |
| | catabolism | | | | | | | | |
| | aromatic amiNumber acid family | 9 | −1.038 | 0 | 0 | −4.15 | 4 | 0.008957 | 8.606557377 |
| | metabolism | | | | | | | | |
| | DNA replication initiation | 10 | 0.688 | 2.75 | 4 | 0 | 0 | 0.012315375 | 7.745901639 |
| | regulation of translation | 22 | 0.135 | 1.88 | 4 | −1.07 | 2 | 0.004420544 | 5.281296572 |
| | ribosome biogenesis | 40 | 0.750 | 7.5 | 10 | 0 | 0 | 0.000145834 | 4.841188525 |
| | ribosome biogenesis and assembly | 41 | 0.750 | 7.5 | 10 | 0 | 0 | 0.000178594 | 4.723110756 |
| | DNA dependent DNA replication | 25 | 0.596 | 2.98 | 5 | 0 | 0 | 0.036826074 | 3.87295082 |
| | aromatic compound metabolism | 36 | −0.503 | 1.6 | 1 | −5.12 | 6 | 0.009224943 | 3.765368852 |
| | posttranslational membrane targeting | 39 | 0.491 | 4.71 | 5 | −1.27 | 2 | 0.013591927 | 3.475725095 |
| | cell ion homeostasis | 28 | −0.506 | 0.55 | 1 | −3.08 | 4 | 0.052913392 | 3.457991803 |
| | ER organization and biogenesis | 45 | 0.483 | 5.13 | 6 | −1.27 | 2 | 0.007403407 | 3.442622951 |
| | protein-ER targeting | 45 | 0.483 | 5.13 | 6 | −1.27 | 2 | 0.007403407 | 3.442622951 |
| | protein-membrane targeting | 45 | 0.491 | 4.71 | 5 | −1.27 | 2 | 0.026288289 | 3.012295082 |
| | amiNumber acid metabolism | 59 | −0.80 | 0 | 0 | −6.4 | 8 | 0.030340957 | 2.625729369 |
| | macromolecule biosynthesis | 210 | 0.608 | 18.1 | 26 | −1.07 | 2 | 6.91018E−06 | 2.581967213 |
| | protein biosynthesis | 210 | 0.608 | 18.1 | 26 | −1.07 | 2 | 6.91018E−06 | 2.581967213 |
| | carboxylic acid metabolism | 137 | −0.547 | 0.9 | 2 | −10.2 | 15 | 0.001599216 | 2.402925691 |
| | organic acid metabolism | 138 | −0.547 | 0.9 | 2 | −10.2 | 15 | 0.001727258 | 2.385513186 |
| | cytoplasm organization and biogenesis | 290 | 0.656 | 21.32 | 25 | −2.29 | 4 | 0.000779106 | 1.93647541 |
| | cell organization and biogenesis | 378 | 0.634 | 25.11 | 32 | −2.29 | 4 | 0.00037247 | 1.844262295 |
| | biosynthesis | 413 | 0.360 | 19.82 | 30 | −5.79 | 9 | 0.000231323 | 1.828632954 |
| | death | 167 | 0.523 | 9.6 | 13 | −1.75 | 2 | 0.047103405 | 1.739349171 |
| | cell adhesion | 224 | 0.609 | 13.41 | 18 | −1.24 | 2 | 0.020497695 | 1.728995902 |
| | immune response | 212 | 0.994 | 17.9 | 18 | 0 | 0 | 0.043909246 | 1.644177235 |
| | defense response | 271 | 0.895 | 20.58 | 23 | 0 | 0 | 0.020898098 | 1.643503115 |
| | response to biotic stimulus | 295 | 0.877 | 21.04 | 24 | 0 | 0 | 0.028098496 | 1.575437622 |
| | response to external stimulus | 395 | 0.803 | 23.64 | 28 | −0.34 | 1 | 0.048231031 | 1.421716124 |
| | cell growth and/or maintenance | 1518 | 0.309 | 49.2 | 74 | −18.64 | 25 | 0.003473821 | 1.262918746 |
| | protein metabolism | 1000 | 0.542 | 40.04 | 57 | −4.84 | 8 | 0.027923077 | 1.258709016 |
| | cellular process | 2484 | 0.342 | 72.57 | 111 | −23.97 | 31 | 0.046010892 | 1.107002851 |
| | physiological processes | 3887 | 0.342 | 110.01 | 162 | −37.2 | 51 | 0.019791016 | 1.061150662 |
| DisConcordance | insulin-like growth factor binding | 12 | | | | | | | |
| | organic cation transporter activity | 13 | | | | | | | |
| | growth factor binding | 22 | | | | | | | |
| | heparin binding | 37 | | | | | | | |
| | glycosamiNumberglycan binding | 43 | | | | | | | |
| | cation transporter activity | 88 | | | | | | | |
| | extracellular space | 1093 | | | | | | | |
| | one-carbon compound metabolism | 17 | | | | | | | |
| | angiogenesis | 32 | | | | | | | |
| | regulation of cell growth | 27 | | | | | | | |
| | actin cytoskeleton organization and | 21 | | | | | | | |
| | biogenesis | | | | | | | | |
| | blood vessel development | 35 | | | | | | | |
| | cell growth | 39 | | | | | | | |
| | actin filament-based process | 24 | | | | | | | |
| | enzyme linked receptor protein | 91 | | | | | | | |
| | signaling pathway | | | | | | | | |
| | organelle organization and biogenesis | 248 | | | | | | | |
| | orgaNumbergenesis | 429 | | | | | | | |
| | morphogenesis | 458 | | | | | | | |
| | Experiment Cons. | | 80% up | | | | | | |
| | | | 20% dn | | | | | | |

Discordance

| | | | Average | | | |
|---|---|---|---|---|---|---|
| Average | Average | Number of | Expression | Number of | | |
| Expression | Expression UP | Genes | Down | Genes | EASE | Enrichment |
| 0.088 | 1.74 | 2 | −1.39 | 2 | 0.0006 | 21.94520548 |
| −0.267 | 0.38 | 1 | −1.18 | 2 | 0.0155 | 15.19283456 |
| 0.088 | 1.74 | 2 | −1.39 | 2 | 0.004 | 11.97011208 |
| 0.102 | 2.31 | 3 | −1.8 | 2 | 0.0021 | 8.896704924 |
| 0.102 | 2.31 | 3 | −1.8 | 2 | 0.0037 | 7.655304237 |
| −0.446 | 0.38 | 1 | −2.61 | 4 | 0.0421 | 3.740660025 |
| 0.084 | 9.48 | 12 | −7.47 | 12 | 0.0496 | 1.430619091 |
| −0.517 | 0 | 0 | −1.55 | 3 | 0.0269 | 11.42224013 |
| 0.390 | 2.53 | 3 | −0.58 | 2 | 0.0013 | 10.11344178 |
| 0.088 | 1.74 | 2 | −1.39 | 2 | 0.0076 | 9.589041096 |
| 0.177 | 0.88 | 2 | −0.35 | 1 | 0.0399 | 9.246575342 |
| 0.390 | 2.53 | 3 | −0.58 | 2 | 0.0018 | 9.246575342 |
| −0.018 | 1.74 | 2 | −1.83 | 3 | 0.0027 | 8.298208641 |
| 0.177 | 0.88 | 2 | −0.35 | 1 | 0.0509 | 8.090753425 |
| 0.226 | 1.65 | 3 | −0.52 | 2 | 0.0491 | 3.556375132 |
| −0.216 | 1.43 | 3 | −3.37 | 6 | 0.0336 | 2.348928414 |
| 0.248 | 5.92 | 7 | −2.7 | 6 | 0.0272 | 1.96139477 |
| 0.248 | 5.92 | 7 | −2.7 | 6 | 0.0422 | 1.837201651 |
| 64% up 36% dn | | | | | | |

TABLE 12

| | | Changed genes | Changed genes | P Value | Changed genes | P Value |
|---|---|---|---|---|---|---|
| 1 | All data | 1325 | N.A. | | N.A. | |
| 2 | Both early & late time | 323 | 93 | 0.0001 | 20 | 0.9438 |
| | points (*) | | | | | |
| 3 | Early time point (A) | 629 | 114 | 0.0182 | 35 | 0.3757 |
| 4 | Late time point (B) | 373 | 71 | 0.3105 | 28 | 0.2972 |
| 5 | Up regulated | 802 | 209 | <0.0001 | 30 | <0.0001 |
| 6 | Down regulated | 523 | 69 | <0.0001 | 53 | <0.0001 |
| 7 | Regeneration/RCC: | 278 | 278 | 0 | 0 | <0.0001 |
| | Concordant | | | | | |
| 8 | Regeneration/RCC: | 83 | 0 | <0.0001 | 83 | 0 |
| | Disconcordant | | | | | |
| 9 | Rest of the Data | 964 | 0 | 0 | 0 | 0 |
| 10 | VHL pathway | 104 | 59 | 0 | 16 | 0.0001 |
| 11 | Hypoxia pathway | 95 | 35 | 0.0001 | 16 | <0.0001 |
| 12 | HRE target (HIF) | 17 | 4 | 0.968 | 7 | <0.0001 |
| 13 | IGF pathway | 37 | 9 | 0.7628 | 8 | 0.0003 |
| 14 | Myc pathway | 136 | 55 | <0.0001 | 10 | 0.714 |
| 15 | p53 pathway | 262 | 80 | <0.0001 | 32 | <0.0001 |
| 16 | NF-kB pathway | 52 | 19 | 0.0083 | 5 | 0.4681 |
| 17 | pattern-1 | 225 | 32 | 0.0132 | 15 | 0.8808 |
| 18 | pattern-2 | 192 | 57 | 0.0008 | 2 | 0.0021 |
| 19 | pattern-3 | 51 | 10 | 0.9856 | 5 | 0.4331 |
| 20 | pattern-4 | 37 | 13 | 0.0419 | 0 | 0.213 |
| 21 | pattern-5 | 187 | 38 | 0.9708 | 8 | 0.3031 |
| 22 | pattern-6 | 83 | 27 | 0.0075 | 7 | 0.531 |
| 23 | pattern-7 | 18 | 3 | 0.9119 | 2 | 0.7092 |
| 24 | pattern-8 | 136 | 27 | 0.9346 | 7 | 0.7165 |
| 25 | pattern-9 | 10 | 1 | 0.6659 | 0 | 0.872 |
| 26 | pattern-10 | 41 | 6 | 0.4547 | 5 | 0.2006 |
| 27 | pattern-11 | 45 | 4 | 0.0759 | 9 | 0.0003 |
| 28 | pattern-12 | 36 | 11 | 0.1906 | 0 | 0.223 |
| 29 | pattern-13 | 3 | 0 | | 0 | |
| 30 | pattern-14 | 32 | 13 | 0.0083 | 0 | 0.2688 |
| 31 | pattern-15 | 19 | 4 | 0.8219 | 2 | 0.7615 |
| 32 | pattern-16 | 86 | 6 | 0.002 | 14 | 0.0001 |
| 33 | pattern-17 | 6 | 0 | | 0 | |
| 34 | pattern-18 | 13 | 1 | 0.4216 | 2 | 0.4254 |
| 35 | pattern-19 | 26 | 3 | 0.3697 | 0 | 0.3589 |
| 36 | pattern-20 | 6 | 1 | | 0 | |
| 37 | pattern-21 | 2 | 0 | | 0 | |
| 38 | pattern-22 | 3 | 0 | | 0 | |
| 39 | pattern-23 | 6 | 2 | | 1 | |
| 40 | pattern-24 | 3 | 1 | | 0 | |
| 41 | pattern-25 | 1 | 0 | | 0 | |
| 42 | pattern-26 | 1 | 0 | | 0 | |
| 43 | pattern-27 | 1 | 0 | | 0 | |

| | | Changed genes | P Value | Changed genes | P Value | Changed genes | P Value |
|---|---|---|---|---|---|---|---|
| 1 | All data | N.A. | | N.A. | | N.A. | |
| 2 | Both early & late time | 210 | 0.0004 | 323 | 0 | 0 | 0 |
| | points (*) | | | | | | |
| 3 | Early time point (A) | 480 | 0.0068 | 0 | 0 | 629 | 0 |
| 4 | Late time point (B) | 274 | 0.7706 | 0 | 0 | 0 | 0 |
| 5 | Up regulated | 563 | 0.0116 | 189 | 0.4317 | 336 | <0.0001 |
| 6 | Down regulated | 401 | 0.0116 | 134 | 0.4317 | 293 | <0.0001 |
| 7 | Regeneration/RCC: | 0 | 0 | 93 | 0.0001 | 114 | 0.0182 |
| | Concordant | | | | | | |
| 8 | Regeneration/RCC: | 0 | 0 | 20 | 0.9438 | 35 | 0.3757 |
| | Disconcordant | | | | | | |
| 9 | Rest of the Data | 964 | 0 | 210 | 0.0004 | 480 | 0.0068 |
| 10 | VHL pathway | 29 | 0 | 28 | 0.6094 | 50 | 0.9788 |
| 11 | Hypoxia pathway | 44 | <0.0001 | 24 | 0.9325 | 50 | 0.3478 |
| 12 | HRE target (HIF) | 6 | 0.0012 | 2 | 0.3499 | 12 | 0.0936 |
| 13 | IGF pathway | 20 | 0.0162 | 10 | 0.852 | 19 | 0.7547 |
| 14 | Myc pathway | 71 | <0.0001 | 39 | 0.2596 | 61 | 0.5789 |
| 15 | p53 pathway | 150 | <0.0001 | 69 | 0.4568 | 112 | 0.1009 |
| 16 | NF-kB pathway | 28 | 0.003 | 19 | 0.0549 | 21 | 0.3668 |
| 17 | pattern-1 | 178 | 0.0362 | 96 | <0.0001 | 122 | 0.1102 |
| 18 | pattern-2 | 133 | 0.2018 | 109 | 0 | 76 | 0.005 |
| 19 | pattern-3 | 36 | 0.7772 | 9 | 0.2583 | 39 | 0.0001 |
| 20 | pattern-4 | 24 | 0.3239 | 6 | 0.268 | 31 | <0.0001 |
| 21 | pattern-5 | 141 | 0.5363 | 24 | <0.0001 | 7 | 0 |
| 22 | pattern-6 | 49 | 0.0036 | 29 | 0.0522 | 8 | <0.0001 |
| 23 | pattern-7 | 13 | 0.8685 | 0 | 0.0264 | 7 | 0.5211 |
| 24 | pattern-8 | 102 | 0.7072 | 5 | <0.0001 | 130 | 0 |
| 25 | pattern-9 | 9 | 0.4006 | 3 | 0.9782 | 3 | 0.3681 |
| 26 | pattern-10 | 30 | 0.8709 | 8 | 0.4873 | 1 | <0.0001 |
| 27 | pattern-11 | 32 | 0.8695 | 16 | 0.1545 | 23 | 0.9099 |
| 28 | pattern-12 | 25 | 0.7358 | 9 | 0.8871 | 22 | 0.1989 |
| 29 | pattern-13 | 3 | | 0 | | 0 | |
| 30 | pattern-14 | 19 | 0.1098 | 6 | 0.5051 | 24 | 0.0054 |
| 31 | pattern-15 | 13 | 0.8245 | 0 | 0.0217 | 19 | <0.0001 |
| 32 | pattern-16 | 66 | 0.5323 | 2 | <0.0001 | 79 | <0.0001 |
| 33 | pattern-17 | 6 | | 0 | | 6 | |
| 34 | pattern-18 | 10 | 0.9863 | 0 | 0.0729 | 0 | 0.001 |
| 35 | pattern-19 | 23 | 0.1228 | 0 | 0.0054 | 17 | 0.1408 |
| 36 | pattern-20 | 5 | | 0 | | 5 | |
| 37 | pattern-21 | 2 | | 0 | | 0 | |
| 38 | pattern-22 | 3 | | 0 | | 3 | |
| 39 | pattern-23 | 3 | | 0 | | 0 | |
| 40 | pattern-24 | 2 | | 0 | | 0 | |
| 41 | pattern-25 | 1 | | 0 | | 1 | |
| 42 | pattern-26 | 1 | | 0 | | 1 | |
| 43 | pattern-27 | 1 | | 0 | | 0 | |
| 1 | All data | N.A. | | N.A. | | N.A. | |
| 2 | Both early & late time | 0 | 0 | 189 | 0.4317 | 134 | 0.4317 |
| | points (*) | | | | | | |
| 3 | Early time point (A) | 0 | 0 | 336 | <0.0001 | 293 | <0.0001 |
| 4 | Late time point (B) | 373 | 0 | 277 | <0.0001 | 96 | <0.0001 |
| 5 | Up regulated | 277 | <0.0001 | 802 | 0 | 0 | 0 |
| 6 | Down regulated | 96 | <0.0001 | 0 | 0 | 523 | 0 |
| 7 | Regeneration/RCC: | 71 | 0.3105 | 209 | <0.0001 | 69 | <0.0001 |
| | Concordant | | | | | | |
| 8 | Regeneration/RCC: | 28 | 0.2972 | 30 | <0.0001 | 53 | <0.0001 |
| | Disconcordant | | | | | | |
| 9 | Rest of the Data | 274 | 0.7706 | 563 | 0.0116 | 401 | 0.0116 |
| 10 | VHL pathway | 26 | 0.5282 | 85 | <0.0001 | 19 | <0.0001 |
| 11 | Hypoxia pathway | 21 | 0.2144 | 63 | 0.2762 | 32 | 0.2762 |
| 12 | HRE target (HIF) | 3 | 0.4852 | 10 | 0.9163 | 7 | 0.9163 |
| 13 | IGF pathway | 8 | 0.4775 | 25 | 0.4728 | 12 | 0.4728 |
| 14 | Myc pathway | 36 | 0.7193 | 113 | <0.0001 | 23 | <0.0001 |
| 15 | p53 pathway | 81 | 0.3009 | 199 | <0.0001 | 63 | <0.0001 |
| 16 | NF-kB pathway | 12 | 0.5011 | 43 | 0.0014 | 9 | 0.0014 |
| 17 | pattern-1 | 7 | <0.0001 | 0 | 0 | 225 | 0 |
| 18 | pattern-2 | 7 | <0.0001 | 192 | 0 | 0 | 0 |
| 19 | pattern-3 | 3 | 0.0018 | 0 | 0 | 51 | 0 |
| 20 | pattern-4 | 0 | 0.0006 | 37 | <0.0001 | 0 | <0.0001 |
| 21 | pattern-5 | 156 | 0 | 181 | 0 | 6 | 0 |
| 22 | pattern-6 | 46 | <0.0001 | 83 | <0.0001 | 0 | <0.0001 |
| 23 | pattern-7 | 11 | 0.0012 | 11 | 0.9139 | 7 | 0.9139 |
| 24 | pattern-8 | 1 | <0.0001 | 135 | 0 | 1 | 0 |
| 25 | pattern-9 | 4 | 0.4865 | 0 | 0.0004 | 10 | 0.0004 |
| 26 | pattern-10 | 32 | <0.0001 | 0 | <0.0001 | 41 | <0.0001 |
| 27 | pattern-11 | 6 | 0.0843 | 0 | <0.0001 | 45 | <0.0001 |
| 28 | pattern-12 | 5 | 0.155 | 36 | <0.0001 | 0 | <0.0001 |
| 29 | pattern-13 | 3 | | 0 | | 3 | |
| 30 | pattern-14 | 2 | 0.0203 | 32 | <0.0001 | 0 | <0.0001 |
| 31 | pattern-15 | 0 | 0.0213 | 19 | 0.0007 | 0 | 0.0007 |
| 32 | pattern-16 | 5 | <0.0001 | 5 | 0 | 81 | 0 |
| 33 | pattern-17 | 0 | | 0 | | 6 | |
| 34 | pattern-18 | 13 | <0.0001 | 0 | <0.0001 | 13 | <0.0001 |
| 35 | pattern-19 | 9 | 0.3918 | 17 | 0.6832 | 9 | 0.6832 |
| 36 | pattern-20 | 1 | | 0 | | 6 | |
| 37 | pattern-21 | 2 | | 1 | | 1 | |
| 38 | pattern-22 | 0 | | 3 | | 0 | |
| 39 | pattern-23 | 6 | | 0 | | 6 | |
| 40 | pattern-24 | 3 | | 3 | | 0 | |
| 41 | pattern-25 | 0 | | 1 | | 0 | |
| 42 | pattern-26 | 0 | | 0 | | 1 | |
| 43 | pattern-27 | 1 | | 0 | | 1 | |

TABLE 14

| | | | | | | Cluster/ | |
|---|---|---|---|---|---|---|---|
| | Ischemic | day 1 | day 2 | day 5 | day 14 | Trend | Title |
| 1 | 0.9816 | 0.8677 | 0.7747 | 0.8710 | 0.8696 | 1 | potassium channel, subfamily K, member 2 |
| 2 | 0.9090 | 0.7764 | 0.6622 | 0.8083 | 0.7585 | 1 | ESTs |
| 3 | 0.8806 | 0.5878 | 0.4266 | 0.6908 | 0.6833 | 1 | RIKEN cDNA 1300002P22 gene |
| 4 | 0.9697 | 0.7737 | 0.6545 | 0.8417 | 0.8394 | 1 | DNA segment, Chr 8, Brigham & Women's Genetics 1320 expressed |
| 5 | 1.1098 | 0.8817 | 0.7895 | 0.9195 | 0.9014 | 1 | yolk sac gene 2 |
| 6 | 1.0931 | 0.8849 | 0.8035 | 0.9534 | 0.9308 | 1 | RIKEN cDNA 2310067B10 gene |
| 7 | 0.8617 | 0.2861 | 0.2295 | 0.4066 | 0.4316 | 1 | stearoyl-Coenzyme A desaturase 1 |
| 8 | 0.9097 | 0.6450 | 0.5914 | 0.7186 | 0.7172 | 1 | malonyl-CoA decarboxylase |
| 9 | 1.0502 | 0.7581 | 0.7003 | 0.8569 | 0.8913 | 1 | Mus musculus evectin-2 (Evt2) mRNA, complete cds |
| 10 | 0.8590 | 0.7195 | 0.6667 | 0.7747 | 0.7828 | 1 | lectin, galactose binding, soluble 4 |
| 11 | 1.0703 | 0.8504 | 0.8115 | 1.0596 | 0.8887 | 1 | Mus musculus, Similar to KIAA0763 gene product, clone IMAGE: 4503056, |
| | | | | | | | mRNA, partial cds |
| 12 | 0.9683 | 0.7420 | 0.6598 | 0.9255 | 0.8185 | 1 | Unknown |
| 13 | 1.0738 | 0.8411 | 0.7912 | 1.0231 | 0.9023 | 1 | ESTs |
| 14 | 0.9736 | 0.8005 | 0.7804 | 0.9101 | 0.8200 | 1 | RIKEN cDNA 6430559E15 gene |
| 15 | 1.0206 | 0.7118 | 0.6408 | 0.8797 | 0.7251 | 1 | carnitine palmitoyltransferase 1, muscle |
| 16 | 0.9741 | 0.7476 | 0.6836 | 0.8187 | 0.7625 | 1 | protein C |
| 17 | 1.1201 | 0.7899 | 0.7046 | 0.9285 | 0.7863 | 1 | RIKEN cDNA 1810036E22 gene |
| 18 | 0.9439 | 0.8687 | 0.8369 | 0.9000 | 0.8669 | 1 | cartilage oligomeric matrix protein |
| 19 | 0.9697 | 0.3924 | 0.4049 | 0.6005 | 0.4827 | 1 | reduced in osteosclerosis transporter |
| 20 | 0.9287 | 0.6604 | 0.6645 | 0.8186 | 0.7432 | 1 | insulin-like growth factor binding protein 1 |
| 21 | 0.9338 | 0.5959 | 0.6340 | 0.7963 | 0.6981 | 1 | succinate dehydrogenase complex, subunit A, flavoprotein (Fp) |
| 22 | 0.9549 | 0.5844 | 0.5514 | 0.7331 | 0.6677 | 1 | Mus musculus, similar to quinone reductase-like protein, clone IMAGE: |
| | | | | | | | 4972406, mRNA, partial cds |
| 23 | 0.9978 | 0.6934 | 0.6606 | 0.8285 | 0.7812 | 1 | expressed sequence AI507121 |
| 24 | 0.9025 | 0.6381 | 0.5778 | 0.7577 | 0.7155 | 1 | cytochrome c oxidase, subunit VIIa 1 |
| 25 | 1.0040 | 0.8389 | 0.7995 | 0.9240 | 0.8721 | 1 | tenascin XB |
| 26 | 1.0503 | 0.8404 | 0.8149 | 0.9909 | 0.9303 | 1 | RNA polymerase II 1 |
| 27 | 1.0104 | 0.7286 | 0.6963 | 0.8945 | 0.8229 | 1 | RIKEN cDNA 2610007A16 gene |
| 28 | 1.0255 | 0.8597 | 0.8484 | 0.9682 | 0.9195 | 1 | DNA segment, Chr 4, Wayne State University 125, expressed |
| 29 | 1.2306 | 0.5853 | 0.4562 | 0.9206 | 0.8311 | 1 | betaine-homocysteine methyltransferase |
| 30 | 1.1339 | 0.8985 | 0.8673 | 1.0241 | 1.0013 | 1 | phosphofructokinase, liver, B-type |
| 31 | 1.1378 | 0.9208 | 0.7910 | 0.9501 | 1.0191 | 1 | RIKEN cDNA 9130022E05 gene |
| 32 | 0.8210 | 0.4811 | 0.2679 | 0.4326 | 0.6001 | 1 | cytochrome P450, 2a4 |
| 33 | 1.0851 | 0.8315 | 0.5868 | 0.7763 | 0.9361 | 1 | solute carrier family 22 (organic cation transporter)-like 2 |
| 34 | 1.0287 | 0.9225 | 0.8590 | 0.9075 | 1.0134 | 1 | expressed sequence AI315037 |
| 35 | 0.9210 | 0.7445 | 0.6909 | 0.7575 | 0.8569 | 1 | succinate-Coenzyme A ligase, ADP-forming, beta subunit |
| 36 | 1.0434 | 0.7947 | 0.6915 | 0.8247 | 0.9446 | 1 | interleukin 11 receptor, alpha chain 1 |
| 37 | 0.8544 | 0.4981 | 0.3620 | 0.4663 | 0.7053 | 1 | prolactin receptor related sequence 1 |
| 38 | 0.8627 | 0.7794 | 0.7303 | 0.7622 | 0.8158 | 1 | ectonucleoside triphosphate diphosphohydrolase 5 |
| 39 | 0.9799 | 0.5516 | 0.5815 | 0.6525 | 0.8120 | 1 | RIKEN cDNA 0610025I19 gene |
| 40 | 1.1516 | 0.6399 | 0.6764 | 0.7652 | 0.9557 | 1 | creatine kinase, brain |
| 41 | 0.9616 | 0.4203 | 0.4189 | 0.4665 | 0.6330 | 1 | deiodinase, iodothyronine, type I |
| 42 | 0.9403 | 0.6639 | 0.6705 | 0.7125 | 0.7930 | 1 | Mus musculus chemokine receptor CCX CKR mRNA, complete cds, |
| | | | | | | | alternatively spliced |
| 43 | 0.9686 | 0.6042 | 0.5819 | 0.6591 | 0.7671 | 1 | N-myc downstream regulated 2 |
| 44 | 1.0803 | 0.7817 | 0.7801 | 0.8477 | 0.9472 | 1 | H2B histone family, member S |
| 45 | 0.9561 | 0.5775 | 0.5064 | 0.6518 | 0.7307 | 1 | glycine amidinotransferase (L-arginine:glycine amidinotransferase) |
| 46 | 0.7850 | 0.2953 | 0.2484 | 0.3795 | 0.5106 | 1 | thyroid hormone responsive SPOT14 homolog (Rattus) |
| 47 | 1.0782 | 0.8615 | 0.8179 | 0.9079 | 0.9736 | 1 | ESTs |
| 48 | 1.0587 | 0.7758 | 0.7499 | 0.8548 | 0.9499 | 1 | expressed sequence C79732 |
| 49 | 0.9820 | 0.6923 | 0.6461 | 0.7430 | 0.8694 | 1 | microtubule-associated protein tau |
| 50 | 0.9618 | 0.7034 | 0.6747 | 0.7329 | 0.8453 | 1 | methylmalonyl-Coenzyme A mutase |
| 51 | 0.9158 | 0.3346 | 0.3046 | 0.3854 | 0.6587 | 1 | calbindin-28K |
| 52 | 0.9378 | 0.6674 | 0.6524 | 0.7042 | 0.8523 | 1 | Mus musculus, clone MGC: 19042 IMAGE: 4188988, mRNA, complete cds |
| 53 | 0.9370 | 0.5155 | 0.4658 | 0.5221 | 0.6916 | 1 | Mus musculus, guanine nucleotide binding protein (G protein), gamma 5, |
| | | | | | | | clone MGC: 8292 IMAGE: 3593324, mRNA, complete cds |
| 54 | 0.8953 | 0.6357 | 0.5800 | 0.6558 | 0.7498 | 1 | ESTs |
| 55 | 1.0914 | 0.9025 | 0.8354 | 0.9409 | 1.0999 | 1 | RIKEN cDNA 1200016G03 gene |
| 56 | 0.8811 | 0.5119 | 0.4372 | 0.6067 | 0.7780 | 1 | RIKEN cDNA 1200014D15 gene |
| 57 | 1.0235 | 0.8414 | 0.7692 | 0.8871 | 1.0012 | 1 | ESTs, Weakly similar to S65210 hypothetical protein YPL191c - yeast |
| | | | | | | | (Saccharomyces cerevisiae) (S. cerevisiae) |
| 58 | 1.0699 | 0.8933 | 0.8374 | 0.9557 | 1.0522 | 1 | phosphodiesterase 1A, calmodulin-dependent |
| 59 | 1.1476 | 0.8728 | 0.8572 | 0.9278 | 1.1484 | 1 | RIKEN cDNA 5730403B10 gene |
| 60 | 0.8894 | 0.7555 | 0.7420 | 0.8056 | 0.8780 | 1 | Mus musculus, Similar to chromosome 20 open reading flame 36, clone IMAGE: |
| | | | | | | | 5356821, mRNA, partial cds |
| 61 | 1.0316 | 0.8506 | 0.8489 | 0.9242 | 1.0091 | 1 | RIKEN cDNA 5830445O15 gene |
| 62 | 0.9716 | 0.8073 | 0.8032 | 0.8679 | 0.9415 | 1 | Mus musculus, clone IMAGE: 3967158, mRNA, partial cds |
| 63 | 0.9113 | 0.3797 | 0.3945 | 0.5947 | 0.9574 | 1 | expressed sequence AW146047 |
| 64 | 1.0649 | 0.7988 | 0.8434 | 0.9302 | 1.1040 | 1 | ESTs |
| 65 | 0.9488 | 0.6713 | 0.6895 | 0.7771 | 1.0326 | 1 | DnaJ (Hsp40) homolog, subfamily A, member 1 |
| 66 | 1.0821 | 0.7559 | 0.7927 | 0.9098 | 1.1743 | 1 | solute carrier family 25 (mitochondrial deoxynucleotide carrier), member 19 |
| 67 | 0.9277 | 0.3999 | 0.5456 | 0.5864 | 0.8842 | 1 | ESTs |
| 68 | 0.7433 | 0.3432 | 0.4695 | 0.5011 | 0.7191 | 1 | carboxylesterase 3 |
| 69 | 0.9209 | 0.4518 | 0.5165 | 0.6056 | 0.8343 | 1 | isovaleryl coenzyme A dehydrogenase |
| 70 | 1.0652 | 0.6909 | 0.7498 | 0.8234 | 1.0113 | 1 | interferon inducible protein 1 |
| 71 | 0.8915 | 0.1457 | 0.2289 | 0.3117 | 0.6495 | 1 | Unknown |
| 72 | 0.8809 | 0.5080 | 0.5873 | 0.6507 | 0.8163 | 1 | hydroxysteroid dehydrogenase-3, delta<5>-3-beta |
| 73 | 1.0907 | 0.7718 | 0.8119 | 0.8499 | 1.0203 | 1 | expressed sequence AI875199 |
| 74 | 0.9767 | 0.7984 | 0.8125 | 0.8554 | 0.9502 | 1 | expressed sequence AU018056 |
| 75 | 1.0857 | 0.2240 | 0.3635 | 0.4414 | 0.6803 | 1 | elafin-like protein I |
| 76 | 1.1659 | 0.5582 | 0.7268 | 0.7803 | 0.9661 | 1 | mitochondrial ribosomal protein L39 |
| 77 | 0.9526 | 0.5696 | 0.6423 | 0.7257 | 0.8023 | 1 | RIKEN cDNA 9530058B02 gene |
| 78 | 0.9184 | 0.6949 | 0.7318 | 0.7823 | 0.8551 | 1 | expressed sequence AW493985 |
| 79 | 1.0714 | 0.6146 | 0.7393 | 0.7891 | 0.8486 | 1 | cell death-inducing DNA fragmentation factor, alpha subunit-like effector B |
| 80 | 0.7269 | 0.3202 | 0.3907 | 0.4495 | 0.4816 | 1 | thioether S-methyltransferase |
| 81 | 0.8850 | 0.5453 | 0.6162 | 0.6336 | 0.7483 | 1 | solute carrier family 25 (mitochondrial carrier; adenine nucleotide |
| | | | | | | | translocator), member 10 |
| 82 | 1.1340 | 0.3775 | 0.4685 | 0.5637 | 0.7175 | 1 | ketohexokinase |
| 83 | 1.0887 | 0.6004 | 0.6693 | 0.7303 | 0.8260 | 1 | RIKEN cDNA 2310009E04 gene |
| 84 | 1.0629 | 0.7227 | 0.7162 | 0.8724 | 0.9535 | 1 | RIKEN cDNA 1010001M04 gene |
| 85 | 0.9264 | 0.4762 | 0.4583 | 0.6724 | 0.7798 | 1 | cytochrome P450, 2d10 |
| 86 | 1.0992 | 0.4295 | 0.4052 | 0.6877 | 0.8275 | 1 | expressed sequence AI182282 |
| 87 | 1.0641 | 0.4867 | 0.5117 | 0.7757 | 0.8382 | 1 | Mus musculus, Similar to retinol dehydrogenase type 6, clone MGC: 25965 |
| | | | | | | | IMAGE: 4239862, mRNA, complete cds |
| 88 | 0.9683 | 0.4328 | 0.4633 | 0.6991 | 0.7641 | 1 | RIKEN cDNA 2310032J20 gene |
| 89 | 0.7875 | 0.5083 | 0.5101 | 0.6495 | 0.7127 | 1 | ESTs, Moderately similar to S12207 hypothetical protein (M. musculus) |
| 90 | 1.0246 | 0.8115 | 0.8148 | 0.9413 | 0.9727 | 1 | DnaJ (Hsp40) homolog, subfamily B, member 12 |
| 91 | 0.9827 | 0.7041 | 0.6982 | 0.8583 | 0.8985 | 1 | RIKEN cDNA 1700028A24 gene |
| 92 | 0.7319 | 0.3133 | 0.3233 | 0.5017 | 0.6523 | 1 | lipoprotein lipase |
| 93 | 0.6989 | 0.5380 | 0.5438 | 0.6309 | 0.6902 | 1 | RIKEN cDNA 2810473M14 gene |
| 94 | 0.9782 | 0.7104 | 0.7488 | 0.8607 | 0.9440 | 1 | ESTs |
| 95 | 0.9605 | 0.6353 | 0.6775 | 0.8070 | 0.9296 | 1 | peroxisomal membrane protein 2, 22 kDa |
| 96 | 0.8747 | 0.3931 | 0.4268 | 0.6434 | 0.7513 | 1 | phosphoglycerate mutase 2 |
| 97 | 0.9680 | 0.7001 | 0.7289 | 0.8378 | 0.9105 | 1 | RIKEN cDNA 2310001A20 gene |
| 98 | 1.0413 | 0.5559 | 0.6532 | 0.8301 | 0.8000 | 1 | Mus musculus mRNA for alpha-albumin protein |
| 99 | 0.8523 | 0.5420 | 0.6286 | 0.7517 | 0.7429 | 1 | flavin containing monooxygenase 1 |
| 100 | 1.1397 | 0.4946 | 0.5457 | 0.7478 | 0.8194 | 1 | Mus musculus adult male liver cDNA, RIKEN full-length enriched library, |
| | | | | | | | clone:1300015E02:deoxyribonuclease II alpha, full insert sequence |
| 101 | 1.0649 | 0.6761 | 0.7263 | 0.8861 | 0.8952 | 1 | Kruppel-like factor 1 (erythroid) |
| 102 | 0.9704 | 0.4954 | 0.4989 | 0.7039 | 0.7189 | 1 | expressed sequence AI593249 |
| 103 | 0.8461 | 0.6683 | 0.6730 | 0.7503 | 0.7608 | 1 | RIKEN cDNA 5031422I09 gene |
| 104 | 1.0160 | 0.3746 | 0.3836 | 0.6615 | 0.6061 | 1 | acetyl-Coenzyme A dehydrogenase, medium chain |
| 105 | 1.0950 | 0.5338 | 0.5663 | 0.7909 | 0.7616 | 1 | Mus musculus, Similar to hypothetical protein FLJ10520, clone MGC: 27888 |
| | | | | | | | IMAGE: 3497792, mRNA, complete cds |
| 106 | 0.8185 | 0.6572 | 0.6766 | 0.7433 | 0.7375 | 1 | expressed sequence AI875557 |
| 107 | 1.0162 | 0.7861 | 0.9020 | 0.7655 | 0.8195 | 1 | secreted and transmembrane 1 |
| 108 | 1.0582 | 0.4757 | 0.7437 | 0.4369 | 0.5688 | 1 | thioesterase, adipose associated |
| 109 | 1.0423 | 0.7539 | 0.8994 | 0.7239 | 0.8026 | 1 | ornithine aminotransferase |
| 110 | 0.9604 | 0.3250 | 0.6123 | 0.3902 | 0.4696 | 1 | phenylalanine hydroxylase |
| 111 | 1.0047 | 0.6246 | 0.7884 | 0.6474 | 0.7453 | 1 | RIKEN cDNA 2010012D11 gene |
| 112 | 0.8286 | 0.5360 | 0.6649 | 0.5854 | 0.6332 | 1 | ESTs, Weakly similar to AF182426 1 arylacetamide deacetylase (R. norvegicus) |
| 113 | 1.0706 | 0.5573 | 0.8174 | 0.6677 | 0.6123 | 1 | crystallin, lamda 1 |
| 114 | 0.9157 | 0.4763 | 0.6420 | 0.5411 | 0.5450 | 1 | talin 2 |
| 115 | 1.0098 | 0.5704 | 0.7483 | 0.6277 | 0.6430 | 1 | solute carrier family 7 (cationic amino acid transporter, y+ system), member 9 |
| 116 | 0.9352 | 0.5887 | 0.6062 | 0.5635 | 0.7256 | 1 | isovaleryl coenzyme A dehydrogenase |
| 117 | 0.7832 | 0.4427 | 0.4693 | 0.4030 | 0.5847 | 1 | lysine oxoglutarate reductase, saccharopine dehydrogenase |
| 118 | 1.1789 | 0.8399 | 0.8531 | 0.7993 | 0.9974 | 1 | carbonic anhydrase 5a, mitochondrial |
| 119 | 0.8469 | 0.5787 | 0.6202 | 0.5833 | 0.6965 | 1 | pantophysin |
| 120 | 0.9086 | 0.5132 | 0.5835 | 0.5214 | 0.6715 | 1 | coagulation factor XIII, beta subunit |
| 121 | 1.0286 | 0.5089 | 0.6087 | 0.5269 | 0.7038 | 1 | serum/glucocorticoid regulated kinase 2 |
| 122 | 0.9886 | 0.6323 | 0.7070 | 0.6208 | 0.7805 | 1 | expressed sequence AU015645 |
| 123 | 1.1261 | 0.5924 | 0.6651 | 0.5452 | 0.7937 | 1 | Mus musculus, clone MGC: 37818 IMAGE: 5098655, mRNA, complete cds |
| 124 | 0.9844 | 0.6231 | 0.7273 | 0.6301 | 0.7563 | 1 | solute carrier family 16 (monocarboxylic acid transporters), member 7 |
| 125 | 1.1712 | 0.5671 | 0.7058 | 0.5264 | 0.7447 | 1 | RIKEN cDNA 1810027P18 gene |
| 126 | 0.9479 | 0.7389 | 0.7905 | 0.7286 | 0.8047 | 1 | RIKEN cDNA 1110038J12 gene |
| 127 | 1.0157 | 0.4696 | 0.7027 | 0.4861 | 0.6971 | 1 | J domain protein 1 |
| 128 | 0.9351 | 0.7323 | 0.8148 | 0.7266 | 0.8336 | 1 | adducin 3 (gamma) |
| 129 | 0.8681 | 0.6479 | 0.6819 | 0.6522 | 0.7914 | 1 | phytanoyl-CoA hydroxylase |
| 130 | 1.0525 | 0.8201 | 0.8850 | 0.8472 | 0.9859 | 1 | Unknown |
| 131 | 1.0470 | 0.3491 | 0.4474 | 0.4476 | 0.7893 | 1 | protein phosphatase 1, regulatory (inhibitor) subunit 1A |
| 132 | 0.8697 | 0.6571 | 0.6847 | 0.6817 | 0.7783 | 1 | ESTs, Weakly similar to DRR1 (H. sapiens) |
| 133 | 0.9008 | 0.6215 | 0.6344 | 0.6362 | 0.7915 | 1 | Rhesus blood group-associated C glycoprotein |
| 134 | 1.0869 | 0.5858 | 0.7381 | 0.6738 | 0.8361 | 1 | RIKEN cDNA 0710008N11 gene |
| 135 | 0.9425 | 0.6240 | 0.6913 | 0.6689 | 0.7877 | 1 | RIKEN cDNA 2410021P16 gene |
| 136 | 0.9033 | 0.0708 | 0.1492 | 0.1233 | 0.3500 | 1 | epidermal growth factor |
| 137 | 1.1972 | 0.6956 | 0.8314 | 0.8082 | 0.9795 | 1 | Mus musculus, Similar to MIPP65 protein, clone MGC: 18783 IMAGE: |
| | | | | | | | 4188234, mRNA, complete cds |
| 138 | 1.0090 | 0.7053 | 0.7495 | 0.7547 | 0.8487 | 1 | enoyl Coenzyme A hydratase, short chain, 1, mitochondrial |
| 139 | 1.0820 | 0.7674 | 0.8403 | 0.8282 | 0.9008 | 1 | RIKEN cDNA 1300017C12 gene |
| 140 | 0.6980 | 0.2962 | 0.3814 | 0.3800 | 0.4743 | 1 | adenylate kinase 4 |
| 141 | 0.9453 | 0.5332 | 0.6121 | 0.6285 | 0.7339 | 1 | transthyretin |
| 142 | 0.9767 | 0.4281 | 0.4910 | 0.4654 | 0.5762 | 1 | klotho |
| 143 | 0.9457 | 0.5191 | 0.5988 | 0.5566 | 0.6680 | 1 | ectonucleotide pyrophosphatase/phosphodiesterase 2 |
| 144 | 0.8730 | 0.2441 | 0.3249 | 0.2815 | 0.4363 | 1 | 4-hydroxyphenylpyruvic acid dioxygenase |
| 145 | 0.9976 | 0.5594 | 0.6852 | 0.6182 | 0.7160 | 1 | growth arrest specific 2 |
| 146 | 0.8908 | 0.5770 | 0.6674 | 0.6105 | 0.6682 | 1 | sterol carrier protein 2, liver |
| 147 | 0.9990 | 0.6529 | 0.8622 | 0.6962 | 0.8702 | 1 | nuclear protein 15.6 |
| 148 | 1.0217 | 0.6998 | 0.8127 | 0.8039 | 0.8309 | 1 | transmembrane protein 8 (five membrane-spanning domains) |
| 149 | 0.8993 | 0.4348 | 0.5856 | 0.5520 | 0.5861 | 1 | nicotinamide nucleotide transhydrogenase |
| 150 | 1.0979 | 0.7508 | 0.8679 | 0.8355 | 0.8613 | 1 | transcription elongation factor A (SII), 3 |
| 151 | 0.9386 | 0.5098 | 0.7191 | 0.6046 | 0.7392 | 1 | solute carrier family 4 (anion exchanger), member 4 |
| 152 | 1.0865 | 0.4908 | 0.6878 | 0.5853 | 0.7315 | 1 | malate dehydrogenase, soluble |
| 153 | 1.0318 | 0.5602 | 0.7579 | 0.6736 | 0.7638 | 1 | folate receptor 1 (adult) |
| 154 | 0.7704 | 0.1985 | 0.3914 | 0.2790 | 0.4076 | 1 | glucose-6-phosphatase, catalytic |
| 155 | 0.8940 | 0.3600 | 0.5677 | 0.5110 | 0.6968 | 1 | RIKEN cDNA 6330565B14 gene |
| 156 | 0.9634 | 0.5947 | 0.7844 | 0.7270 | 0.8165 | 1 | cytochrome P450, 2j5 |
| 157 | 1.0133 | 0.8106 | 0.7664 | 0.7576 | 0.6972 | 1 | dihydropyrimidinase |
| 158 | 0.8802 | 0.5798 | 0.5064 | 0.5414 | 0.4831 | 1 | gamma-glutamyl transpeptidase |
| 159 | 0.9990 | 0.6900 | 0.6239 | 0.6408 | 0.6133 | 1 | solute carrier family 22 (organic cation transporter), member 1 |
| 160 | 1.0002 | 0.6882 | 0.6353 | 0.6282 | 0.6051 | 1 | methylenetetrahydrofolate dehydrogenase (NADP+ dependent), |
| | | | | | | | methenyltetrahydrofolate cyclohydrolase, formyltetrahydrofolate synthase |
| 161 | 0.9077 | 0.7880 | 0.7217 | 0.7266 | 0.7518 | 1 | ESTs |
| 162 | 1.0037 | 0.7300 | 0.6592 | 0.6364 | 0.6690 | 1 | ESTs |
| 163 | 0.9562 | 0.7763 | 0.7292 | 0.7322 | 0.7508 | 1 | RIKEN cDNA 1300004O04 gene |
| 164 | 1.1117 | 0.6548 | 0.6594 | 0.6576 | 0.6527 | 1 | solute carrier family 22 (organic cation transporter), member 2 |
| 165 | 1.0800 | 0.5603 | 0.5244 | 0.4742 | 0.5401 | 1 | transcobalamin 2 |
| 166 | 1.0942 | 0.5996 | 0.5594 | 0.5437 | 0.5630 | 1 | fumarylacetoacetate hydrolase |
| 167 | 1.1004 | 0.7860 | 0.7853 | 0.7628 | 0.7845 | 1 | isocitrate dehydrogenase 2 (NADP+), mitochondrial |
| 168 | 0.8939 | 0.3244 | 0.3173 | 0.2147 | 0.2962 | 1 | deoxyribonuclease I |
| 169 | 0.9275 | 0.5975 | 0.6047 | 0.5280 | 0.5993 | 1 | glutaryl-Coenzyme A dehydrogenase |
| 170 | 1.0114 | 0.7205 | 0.7236 | 0.6446 | 0.7168 | 1 | L-3-hydroxyacyl-Coenzyme A dehydrogenase, short chain |
| 171 | 1.0638 | 0.8670 | 0.8366 | 0.7863 | 0.8366 | 1 | expressed sequence AW045860 |
| 172 | 1.0769 | 0.8877 | 0.8476 | 0.8111 | 0.8685 | 1 | kinase insert domain protein receptor |
| 173 | 0.9862 | 0.8522 | 0.8240 | 0.8077 | 0.8493 | 1 | phosphoglycerate kinase 1 |
| 174 | 1.0240 | 0.6953 | 0.6481 | 0.7282 | 0.6632 | 1 | solute carrier family 13 (sodium-dependent dicarboxylate transporter), member 3 |
| 175 | 0.9576 | 0.7355 | 0.6591 | 0.7139 | 0.7480 | 1 | ATP synthase, H+ transporting, mitochondrial F1 complex, alpha subunit, isoform 1 |
| 176 | 1.2460 | 0.4745 | 0.3326 | 0.4327 | 0.4733 | 1 | kidney-derived aspartic protease-like protein |
| 177 | 1.0102 | 0.7600 | 0.6782 | 0.7534 | 0.7659 | 1 | expressed sequence AI132189 |
| 178 | 1.1204 | 0.8348 | 0.7830 | 0.8549 | 0.8631 | 1 | serologically defined colon cancer antigen 28 |
| 179 | 0.7649 | 0.5348 | 0.4768 | 0.5543 | 0.5549 | 1 | proline dehydrogenase |
| 180 | 1.0314 | 0.8121 | 0.7031 | 0.7603 | 0.7668 | 1 | leucine zipper-EF-hand containing transmembrane protein 1 |
| 181 | 1.0592 | 0.7780 | 0.7070 | 0.7888 | 0.7557 | 1 | Mus musculus, similar to R29893_1, clone MGC: 37808 IMAGE: 5098192, |
| | | | | | | | mRNA, complete cds |
| 182 | 1.3884 | 0.6018 | 0.4223 | 0.5567 | 0.5418 | 1 | Unknown |
| 183 | 1.0022 | 0.8612 | 0.6783 | 0.7389 | 0.8014 | 1 | RIKEN cDNA 5730408C10 gene |
| 184 | 0.8946 | 0.7703 | 0.6541 | 0.6768 | 0.7313 | 1 | ESTs |
| 185 | 1.0201 | 0.8708 | 0.7479 | 0.7935 | 0.8518 | 1 | ESTs, Weakly similar to TYROSINE-PROTEIN KINASE JAK3 (M. musculus) |
| 186 | 0.9130 | 0.7572 | 0.7174 | 0.7053 | 0.7895 | 1 | RIKEN cDNA 9030612M13 gene |
| 187 | 0.8750 | 0.6932 | 0.6513 | 0.6516 | 0.7267 | 1 | ATP-binding cassette, sub-family D (ALD), member 3 |
| 188 | 1.0250 | 0.7788 | 0.7025 | 0.7654 | 0.8520 | 1 | Unknown |
| 189 | 0.9676 | 0.7039 | 0.6232 | 0.6705 | 0.7568 | 1 | glycerol-3-phosphate acyltransferase, mitochondrial |
| 190 | 1.0032 | 0.6663 | 0.5200 | 0.5587 | 0.7215 | 1 | kallikrein 26 |
| 191 | 1.1525 | 0.6470 | 0.4745 | 0.5596 | 0.6527 | 1 | parvalbumin |
| 192 | 1.2349 | 0.8810 | 0.7591 | 0.7995 | 0.9074 | 1 | Unknown |
| 193 | 1.0265 | 0.6755 | 0.8175 | 0.8411 | 0.7119 | 1 | citrate lyase beta like |
| 194 | 1.3176 | 0.4719 | 0.7015 | 0.6765 | 0.5463 | 1 | solute carrier family 34 (sodium phosphate), member 1 |
| 195 | 0.9920 | 0.6257 | 0.7415 | 0.7693 | 0.6849 | 1 | Mus musculus, clone IMAGE: 4974221, mRNA, partial cds |
| 196 | 1.1545 | 0.7438 | 0.8510 | 0.8386 | 0.7072 | 1 | hepsin |
| 197 | 1.1146 | 0.8368 | 0.8779 | 0.8637 | 0.8170 | 1 | Mus musculus, clone MGC: 12039 IMAGE: 3603661, mRNA, complete cds |
| 198 | 1.2015 | 0.5233 | 0.6369 | 0.6225 | 0.5765 | 1 | RIKEN cDNA 4632401C08 gene |
| 199 | 1.0841 | 0.5163 | 0.5927 | 0.5704 | 0.6060 | 1 | dipeptidase 1 (renal) |
| 200 | 1.0379 | 0.6638 | 0.7209 | 0.7349 | 0.7375 | 1 | D-dopachrome tautomerase |
| 201 | 1.0144 | 0.6178 | 0.6537 | 0.6857 | 0.6640 | 1 | Mus musculus, Similar to xylulokinase homolog (H. influenzae), clone |
| | | | | | | | IMAGE: 5043428, mRNA, partial cds |
| 202 | 1.0382 | 0.4725 | 0.5407 | 0.6132 | 0.5281 | 1 | glucose-6-phosphatase, transport protein 1 |
| 203 | 0.9993 | 0.7084 | 0.7611 | 0.8145 | 0.7461 | 1 | expressed sequence AI118577 |
| 204 | 0.9764 | 0.6680 | 0.6875 | 0.7434 | 0.6585 | 1 | ATP synthase, H+ transporting mitochondrial F1 complex, beta subunit |
| 205 | 1.1343 | 0.7213 | 0.7605 | 0.8015 | 0.7336 | 1 | histidyl tRNA synthetase |
| 206 | 1.1628 | 0.4598 | 0.5581 | 0.6376 | 0.5977 | 1 | solute carrier family 22 (organic cation transporter), member 1-like |
| 207 | 0.9297 | 0.5303 | 0.5947 | 0.6322 | 0.6735 | 1 | Rap1, GTPase-activating protein 1 |
| 208 | 1.0080 | 0.6441 | 0.6760 | 0.7477 | 0.7820 | 1 | branched chain aminotransferase 2, mitochondrial |
| 209 | 1.0966 | 0.5961 | 0.6505 | 0.7207 | 0.7840 | 1 | meprin 1 alpha |
| 210 | 1.1247 | 0.7141 | 0.7394 | 0.8393 | 0.8455 | 1 | Unknown |
| 211 | 0.9766 | 0.5290 | 0.5834 | 0.6728 | 0.6687 | 1 | pyruvate dehydrogenase 2 |
| 212 | 1.0056 | 0.5933 | 0.6498 | 0.7343 | 0.7107 | 1 | RIKEN cDNA 4930552N12 gene |
| 213 | 1.0585 | 0.7025 | 0.6965 | 0.7986 | 0.7874 | 1 | malic enzyme, supernatant |
| 214 | 1.0762 | 0.7857 | 0.7670 | 0.8569 | 0.8367 | 1 | PPAR gamma coactivator-1beta protein |
| 215 | 0.9796 | 0.4365 | 0.4333 | 0.5143 | 0.6052 | 1 | Kruppel-like factor 15 |
| 216 | 1.1134 | 0.8427 | 0.8362 | 0.8990 | 0.9549 | 1 | expressed sequence AW124722 |
| 217 | 0.9568 | 0.6968 | 0.6821 | 0.7556 | 0.7712 | 1 | inositol polyphosphate-5-phosphatase, 75 kDa |
| 218 | 0.9549 | 0.7756 | 0.7552 | 0.8198 | 0.8418 | 1 | RIKEN cDNA 5730534O06 gene |
| 219 | 0.9682 | 0.7983 | 0.7872 | 0.8464 | 0.8625 | 1 | Unknown |
| 220 | 0.9909 | 0.7391 | 0.7866 | 0.7394 | 0.7770 | 1 | RIKEN cDNA 2310004L02 gene |
| 221 | 0.9733 | 0.5662 | 0.5830 | 0.5607 | 0.6293 | 1 | Kruppel-like factor 9 |
| 222 | 1.0665 | 0.7345 | 0.7559 | 0.7262 | 0.8011 | 1 | ESTs, Highly similar to organic cation transporter-like protein 2 (M. musculus) |
| 223 | 0.9426 | 0.5861 | 0.6132 | 0.5488 | 0.6436 | 1 | branched chain ketoacid dehydrogenase E1, alpha polypeptide |
| 224 | 0.8393 | 0.5503 | 0.5824 | 0.5344 | 0.5977 | 1 | expressed sequence AI182284 |
| 225 | 0.9097 | 0.6177 | 0.6167 | 0.6402 | 0.6621 | 1 | Mus musculus, clone MGC: 7898 IMAGE: 3582717, mRNA, complete cds |
| 226 | 0.8572 | 0.3460 | 0.3796 | 0.3960 | 0.4323 | 1 | ubiquitin specific protease 2 |
| 227 | 0.9386 | 0.4639 | 0.4980 | 0.5248 | 0.5796 | 1 | hypothetical protein, I54 |
| 228 | 0.8769 | 0.6368 | 0.6346 | 0.6398 | 0.7097 | 1 | Mus musculus, Similar to ubiquitin-conjugating enzyme E2 variant 1, clone |
| | | | | | | | MGC: 7660 IMAGE: 3496088, mRNA, complete cds |
| 229 | 1.0962 | 0.8293 | 0.7960 | 0.8341 | 0.8861 | 1 | expressed sequence AI836219 |
| 230 | 1.1199 | 0.9255 | 0.9011 | 0.9268 | 0.9612 | 1 | ESTs, Weakly similar to YAE6_YEAST HYPOTHETICAL 13.4 KD |
| | | | | | | | PROTEIN IN ACS1-GCV3 INTERGENIC REGION (S. cerevisiae) |
| 231 | 1.1177 | 1.4144 | 1.2884 | 1.2935 | 1.2300 | 2 | RIKEN cDNA 2610206D03 gene |
| 232 | 0.6800 | 2.8720 | 1.6415 | 1.8467 | 1.2875 | 2 | transforming growth factor beta 1 induced transcript 4 |
| 233 | 1.0149 | 1.3398 | 1.2042 | 1.2244 | 1.1310 | 2 | phospholipase A2, activating protein |
| 234 | 0.9134 | 2.8307 | 1.9796 | 1.9638 | 1.4305 | 2 | coagulation factor III |
| 235 | 0.9357 | 1.8019 | 1.4473 | 1.4495 | 1.2616 | 2 | WD repeat domain 1 |
| 236 | 0.9033 | 1.6039 | 1.3419 | 1.3908 | 1.1307 | 2 | Harvey rat sarcoma oncogene, subgroup R |
| 237 | 0.8760 | 2.1221 | 1.5577 | 1.7149 | 1.3271 | 2 | solute carrier family 13 (sodium/sulphate symporters), member 1 |
| 238 | 0.8933 | 1.3513 | 1.1848 | 1.1199 | 1.0507 | 2 | ESTs |
| 239 | 1.0107 | 1.8379 | 1.5108 | 1.4259 | 1.2037 | 2 | lymphocyte antigen 6 complex, locus A |
| 240 | 1.1624 | 1.7770 | 1.5018 | 1.5037 | 1.3295 | 2 | E74-like factor 3 |
| 241 | 0.9602 | 1.5740 | 1.2196 | 1.3172 | 1.1062 | 2 | Mus musculus, clone MGC: 18985 IMAGE: 4011674, mRNA, complete cds |
| 242 | 1.0314 | 1.5023 | 1.2505 | 1.4018 | 1.1581 | 2 | Tnf receptor-associated factor 2 |
| 243 | 0.9591 | 2.0042 | 1.3889 | 1.6818 | 1.3369 | 2 | growth differentiation factor 15 |
| 244 | 0.8665 | 1.5614 | 1.2282 | 1.3507 | 1.3126 | 2 | tumor necrosis factor receptor superfamily, member 1a |
| 245 | 0.7701 | 1.9641 | 1.3683 | 1.6552 | 1.5793 | 2 | zinc finger protein 36, C3H type-like 1 |
| 246 | 0.9826 | 1.6496 | 1.3292 | 1.5357 | 1.4424 | 2 | myelocytomatosis oncogene |
| 247 | 0.8347 | 2.6676 | 1.7628 | 2.2053 | 1.8106 | 2 | a disintegrin-like and metalloprotease (reprolysin type) with thrombospondin type 1 |
| | | | | | | | motif, 1 |
| 248 | 0.8295 | 1.4854 | 1.1421 | 1.3107 | 1.1998 | 2 | calpain 2 |
| 249 | 0.9264 | 2.4502 | 1.8892 | 2.0736 | 2.1824 | 2 | tenascin C |
| 250 | 0.9523 | 2.2413 | 1.8668 | 1.8792 | 1.9387 | 2 | phosphoprotein enriched in astrocytes 15 |
| 251 | 1.0493 | 1.3687 | 1.2938 | 1.3043 | 1.2953 | 2 | cholinergic receptor, nicotinic, beta polypeptide 1 (muscle) |
| 252 | 1.0134 | 1.6451 | 1.6312 | 1.5103 | 1.4454 | 2 | claudin 7 |
| 253 | 0.9392 | 1.3161 | 1.2632 | 1.2284 | 1.2312 | 2 | ESTs |
| 254 | 0.9216 | 2.0534 | 1.8881 | 1.8123 | 1.7364 | 2 | LPS-induced TNF-alpha factor |
| 255 | 0.8604 | 1.3457 | 1.2803 | 1.2674 | 1.2237 | 2 | lysyl oxidase-like |
| 256 | 0.9198 | 1.4348 | 1.3808 | 1.4179 | 1.2070 | 2 | RIKEN cDNA 1110014C03 gene |
| 257 | 1.0637 | 2.2833 | 2.1368 | 2.1240 | 1.8200 | 2 | cystatin B |
| 258 | 1.1002 | 1.6735 | 1.5858 | 1.6359 | 1.4731 | 2 | intercellular adhesion molecule |
| 259 | 0.9795 | 1.3579 | 1.2472 | 1.2478 | 1.1988 | 2 | ADP-ribosylation factor 1 |
| 260 | 0.9126 | 1.5544 | 1.3792 | 1.3364 | 1.3147 | 2 | Mus musculus, clone MGC: 29021 IMAGE: 3495957, mRNA, complete cds |
| 261 | 1.1012 | 2.4132 | 2.0059 | 2.0296 | 1.6679 | 2 | Mus musculus, Similar to transgelin 2, clone MGC: 6300 IMAGE: 2654381, |
| | | | | | | | mRNA, complete cds |
| 262 | 0.8964 | 1.7022 | 1.5114 | 1.3836 | 1.2569 | 2 | Bcl2-interacting killer-like |
| 263 | 1.1238 | 1.5098 | 1.4193 | 1.3938 | 1.3280 | 2 | expressed sequence C87222 |
| 264 | 0.9803 | 1.3292 | 1.1469 | 1.1203 | 1.1531 | 2 | phosphatidylinositol 3-kinase, regulatory subunit, polypeptide 1 (p85 alpha) |
| 265 | 0.8721 | 1.9975 | 1.3200 | 1.3038 | 1.3868 | 2 | heat shock protein, 86 kDa 1 |
| 266 | 0.9617 | 1.3640 | 1.1524 | 1.1427 | 1.1649 | 2 | proteasome (prosome, macropain) subunit, alpha type 6 |
| 267 | 1.0063 | 1.5144 | 1.3115 | 1.1768 | 1.2733 | 2 | RIKEN cDNA 1110001I24 gene |
| 268 | 0.8258 | 2.0001 | 1.4645 | 1.2751 | 1.3404 | 2 | MORF-related gene X |
| 269 | 0.9085 | 1.9206 | 1.5273 | 1.2491 | 1.3807 | 2 | Mus musculus, similar to heterogeneous nuclear ribonucleoprotein A3 |
| | | | | | | | (H. sapiens), clone MGC: 37309 IMAGE: 4975085, mRNA, complete cds |
| 270 | 1.0075 | 1.4756 | 1.3283 | 1.2107 | 1.2584 | 2 | ADP-ribosyltransferase(NAD+; poly (ADP-ribose) polymerase) 2 |
| 271 | 0.8578 | 1.3028 | 1.1672 | 1.0462 | 1.1061 | 2 | heat shock 70 kDa protein 4 |
| 272 | 0.8008 | 2.2114 | 1.8429 | 1.5851 | 1.6467 | 2 | tumor-associated calcium signal transducer 2 |
| 273 | 1.0085 | 1.4867 | 1.3981 | 1.2873 | 1.3456 | 2 | coagulation factor II (thrombin) receptor-like 1 |
| 274 | 1.0238 | 1.3838 | 1.2981 | 1.2288 | 1.2705 | 2 | chloride intracelluar channel 4 (mitochondrial) |
| 275 | 0.8753 | 1.2512 | 1.1707 | 1.0575 | 1.1852 | 2 | SH3 domain protein 3 |
| 276 | 0.9818 | 1.2473 | 1.1897 | 1.1530 | 1.2019 | 2 | adaptor-related protein complex AP-3, sigma 1 subunit |
| 277 | 0.9810 | 1.2570 | 1.1916 | 1.1483 | 1.2259 | 2 | RIKEN cDNA 1200015A22 gene |
| 278 | 1.0146 | 1.4743 | 1.2704 | 1.2796 | 1.3323 | 2 | Mus musculus, Similar to cortactin isoform B, clone MGC: 18474 IMAGE: |
| | | | | | | | 3981559, mRNA, complete cds |
| 279 | 0.9822 | 1.2897 | 1.1758 | 1.1738 | 1.2636 | 2 | RIKEN cDNA 1300013G12 gene |
| 280 | 0.8331 | 1.6366 | 1.5584 | 1.2673 | 1.1268 | 2 | cyclin-dependent kinase 4 |
| 281 | 1.0659 | 2.1308 | 2.0019 | 1.6135 | 1.5434 | 2 | tropomyosin 3, gamma |
| 282 | 1.0687 | 1.9801 | 1.8893 | 1.5845 | 1.4756 | 2 | fibroblast growth factor regulated protein |
| 283 | 0.9989 | 3.9243 | 2.9267 | 2.1458 | 2.0958 | 2 | keratin complex 2, basic, gene 8 |
| 284 | 1.0899 | 4.6727 | 3.7273 | 2.5667 | 2.4503 | 2 | lectin, galactose binding, soluble 3 |
| 285 | 0.9848 | 2.3187 | 2.1390 | 1.8054 | 1.7091 | 2 | serine (or cysteine) proteinase inhibitor, clade H (heat shock protein 47), member 1 |
| 286 | 1.0154 | 1.5290 | 1.4963 | 1.3198 | 1.3474 | 2 | ubiquitin-conjugating enzyme E2I |
| 287 | 1.0560 | 1.4037 | 1.3611 | 1.2613 | 1.2650 | 2 | neural proliferation, differentiation and control gene 1 |
| 288 | 0.9310 | 1.2713 | 1.2741 | 1.0298 | 1.1224 | 2 | GPI-anchored membrane protein 1 |
| 289 | 0.8877 | 1.2020 | 1.1761 | 0.9695 | 1.0258 | 2 | calreticulin |
| 290 | 0.9097 | 1.5046 | 1.4530 | 1.1389 | 1.2200 | 2 | adenylyl cyclase-associated CAP protein homolog 1 (S. cerevisiae, S. pombe) |
| 291 | 0.8963 | 1.2355 | 1.1705 | 1.0284 | 1.1040 | 2 | proteasome (prosome, macropain) 26S subunit, non-ATPase, 10 |
| 292 | 1.1520 | 1.7591 | 1.8477 | 1.4794 | 1.5455 | 2 | v-ral simian leukemia viral oncogene homolog B (ras related) |
| 293 | 0.9901 | 2.0239 | 2.1131 | 1.5391 | 1.5706 | 2 | claudin 1 |
| 294 | 0.8870 | 1.2718 | 1.2727 | 1.0372 | 1.1603 | 2 | glucose regulated protein, 58 kDa |
| 295 | 0.8438 | 1.2329 | 1.2788 | 1.0286 | 1.1318 | 2 | ESTs |
| 296 | 0.8472 | 1.3494 | 1.3412 | 1.1025 | 1.2485 | 2 | mitogen activated protein kinase kinase kinase 1 |
| 297 | 0.9530 | 1.3983 | 1.4666 | 1.1966 | 1.3499 | 2 | testis derived transcript |
| 298 | 1.0267 | 1.2245 | 1.2548 | 1.1265 | 1.1962 | 2 | expressed sequence BBI20430 |
| 299 | 1.1267 | 2.3508 | 2.8522 | 1.9259 | 1.4845 | 2 | actin, alpha 2, smooth muscle, aorta |
| 300 | 1.0701 | 1.3486 | 1.4268 | 1.2728 | 1.1333 | 2 | transformation related protein 53 |
| 301 | 1.0242 | 1.3951 | 1.4901 | 1.3186 | 1.1331 | 2 | TAF10 RNA polymerase II, TATA box binding protein (TBP)-associated |
| | | | | | | | factor, 30 kDa |
| 302 | 1.0327 | 5.5978 | 6.2431 | 4.3856 | 2.3330 | 2 | clusterin |
| 303 | 1.3299 | 2.4505 | 2.6599 | 2.3061 | 1.7330 | 2 | cytokine inducible SH2-containing protein 3 |
| 304 | 0.9466 | 1.3646 | 1.4126 | 1.2365 | 1.1071 | 2 | flotillin 2 |
| 305 | 1.2320 | 2.1492 | 2.2419 | 1.9300 | 1.4928 | 2 | actin-like |
| 306 | 1.0182 | 2.1818 | 2.2685 | 1.8189 | 1.2962 | 2 | cofilin 1, non-muscle |
| 307 | 0.9951 | 1.7838 | 1.9499 | 1.3920 | 1.3150 | 2 | ribosomal protein L6 |
| 308 | 1.0653 | 1.5150 | 1.5837 | 1.2777 | 1.2402 | 2 | ribosomal protein L21 |
| 309 | 1.2079 | 1.6367 | 1.6970 | 1.4678 | 1.3937 | 2 | ras homolog B (RhoB) |
| 310 | 1.0536 | 1.8475 | 2.0800 | 1.5322 | 1.3562 | 2 | guanine nucleotide binding protein, beta 2, related sequence 1 |
| 311 | 1.0999 | 1.5457 | 1.6232 | 1.3656 | 1.2718 | 2 | ribosomal protein S3 |
| 312 | 0.9785 | 2.1319 | 2.1961 | 1.4512 | 1.2421 | 2 | RAN, member RAS oncogene family |
| 313 | 1.0625 | 2.1075 | 2.0691 | 1.5412 | 1.3032 | 2 | zinc finger protein 36, C3H type-like 2 |
| 314 | 1.0773 | 1.3922 | 1.4052 | 1.2814 | 1.1471 | 2 | heparin binding epidermal growth factor-like growth factor |
| 315 | 0.9822 | 1.6328 | 1.5965 | 1.3330 | 1.1288 | 2 | myosin light chain, alkali, cardiac atria |
| 316 | 0.9188 | 1.5654 | 1.5551 | 1.2580 | 1.0350 | 2 | mini chromosome maintenance deficient 4 homolog (S. cerevisiae) |
| 317 | 1.0793 | 5.5524 | 9.3127 | 3.9057 | 2.8346 | 2 | S100 calcium binding protein A6 (calcyclin) |
| 318 | 1.0126 | 1.6739 | 2.0456 | 1.5200 | 1.3133 | 2 | ribosomal protein S3a |
| 319 | 1.0942 | 1.7232 | 2.3267 | 1.5735 | 1.5214 | 2 | ribosomal protein L44 |
| 320 | 1.0637 | 1.8952 | 2.7258 | 1.8208 | 1.5439 | 2 | RNA binding motif protein 3 |
| 321 | 1.0565 | 1.1642 | 1.2306 | 1.1440 | 1.1147 | 2 | Mus musculus, clone MGC: 36997 IMAGE: 4948448, mRNA, complete cds |
| 322 | 1.0705 | 1.7679 | 2.0270 | 1.6345 | 1.5842 | 2 | ribosomal protein S15 |
| 323 | 0.9035 | 1.1124 | 1.2056 | 1.0761 | 1.0596 | 2 | RIKEN cDNA 4933405K01 gene |
| 324 | 0.9504 | 1.2335 | 1.3674 | 1.2804 | 1.1466 | 2 | laminin B1 subunit 1 |
| 325 | 0.9055 | 2.1927 | 3.3491 | 2.2394 | 1.8052 | 2 | RIKEN cDNA 6330583M11 gene |
| 326 | 0.9687 | 1.4965 | 1.8779 | 1.5790 | 1.3338 | 2 | epidermal growth factor-containing fibulin-like extracellular matrix protein 2 |
| 327 | 0.9560 | 1.1582 | 1.1944 | 1.1540 | 1.1070 | 2 | expressed sequence AU015605 |
| 328 | 0.9704 | 1.7327 | 1.9350 | 1.6328 | 1.5458 | 2 | FXYD domain-containing ion transport regulator 5 |
| 329 | 1.0645 | 1.4765 | 1.5744 | 1.4181 | 1.3466 | 2 | urokinase plasminogen activator receptor |
| 330 | 1.0044 | 1.7007 | 1.8942 | 1.6124 | 1.3361 | 2 | ribosomal protein L5 |
| 331 | 0.9628 | 1.4042 | 1.5318 | 1.3774 | 1.2029 | 2 | thymoma viral proto-oncogene 1 |
| 332 | 0.8445 | 1.5391 | 1.8649 | 1.4846 | 1.2736 | 2 | interferon-induced protein with tetratricopeptide repeats 3 |
| 333 | 0.8871 | 1.5872 | 1.7722 | 1.5403 | 1.2828 | 2 | heterogeneous nuclear ribonucleoprotein A1 |
| 334 | 0.9141 | 2.0818 | 2.5192 | 2.0461 | 1.6576 | 2 | heterogeneous nuclear ribonucleoprotein A1 |
| 335 | 1.1017 | 2.0758 | 2.2732 | 2.2015 | 1.5580 | 2 | ESTs Weakly similar to YMP2_CAEEL HYPOTHETICAL 30.3 KD |
| | | | | | | | PROTEIN B0361.2 IN CHROMOSOME III (C. elegans) |
| 336 | 1.0187 | 2.3364 | 2.5172 | 2.3004 | 1.6877 | 2 | chloride intracellular channel 1 |
| 337 | 1.0017 | 1.4357 | 1.4760 | 1.4500 | 1.2531 | 2 | cytidine 5′-triphosphate synthase |
| 338 | 1.0853 | 2.6605 | 2.8033 | 2.1381 | 1.8649 | 2 | tubulin alpha 2 |
| 339 | 1.0494 | 4.1328 | 3.9255 | 2.9854 | 2.2979 | 2 | annexin A2 |
| 340 | 0.9616 | 5.5097 | 5.3863 | 4.4599 | 2.4356 | 2 | transcription elongation regulator 1 (CA150) |
| 341 | 1.0485 | 1.6909 | 1.6517 | 1.5068 | 1.3155 | 2 | ribosomal protein S6 |
| 342 | 1.0107 | 1.1935 | 1.4909 | 1.3491 | 1.2548 | 2 | mammary tumor integration site 6 |
| 343 | 0.9674 | 1.4998 | 2.2714 | 1.8420 | 1.6075 | 2 | ribosomal protein L35 |
| 344 | 0.9967 | 1.1767 | 1.4226 | 1.3022 | 1.2447 | 2 | regulator of G-protein signaling 14 |
| 345 | 0.9704 | 1.3444 | 1.6810 | 1.4334 | 1.4550 | 2 | procollagen, type V, alpha 2 |
| 346 | 0.9739 | 1.2079 | 1.4285 | 1.2661 | 1.2548 | 2 | Unknown |
| 347 | 0.9439 | 1.2135 | 1.3845 | 1.2700 | 1.2523 | 2 | E74-like factor 4 (ets domain transcription factor) |
| 348 | 0.9176 | 1.1151 | 1.2227 | 1.1718 | 1.1249 | 2 | Tial1 cytotoxic granule-associated RNA binding protein-like 1 |
| 349 | 0.9937 | 1.2217 | 1.3762 | 1.2781 | 1.2244 | 2 | TAF9 RNA polymerase II, TATA box binding protein (TBP)-associated |
| | | | | | | | factor, 32 kDa |
| 350 | 1.0739 | 1.6211 | 1.6900 | 1.8066 | 1.3759 | 2 | ribosomal protein L27a |
| 351 | 1.1687 | 1.9212 | 2.0215 | 2.1554 | 1.7325 | 2 | actin, beta, cytoplasmic |
| 352 | 0.9678 | 2.1307 | 2.3285 | 2.9474 | 1.7941 | 2 | secreted acidic cysteine rich glycoprotein |
| 353 | 0.9362 | 1.5474 | 1.7587 | 1.9250 | 1.3770 | 2 | ubiquitin-conjugating enzyme E2H |
| 354 | 0.8998 | 1.3857 | 1.9035 | 1.8941 | 1.6016 | 2 | expressed sequence AW146109 |
| 355 | 0.9329 | 1.1451 | 1.3525 | 1.3079 | 1.2103 | 2 | a disintegrin and metalloproteinase domain 12 (meltrin alpha) |
| 356 | 1.1000 | 1.3553 | 1.4323 | 1.4559 | 1.3386 | 2 | BRG1/brm-associated factor 53A |
| 357 | 1.0509 | 1.3933 | 1.5802 | 1.5723 | 1.4168 | 2 | RIKEN cDNA 4430402G14 gene |
| 358 | 1.0156 | 1.1796 | 1.2639 | 1.2773 | 1.2013 | 2 | Mus musculus, Similar to CGI-147 protein, clone MGC: 25743 IMAGE: |
| | | | | | | | 3990061, mRNA, complete cds |
| 359 | 1.1919 | 1.6059 | 1.9140 | 1.9248 | 1.5416 | 2 | laminin receptor 1 (67 kD, ribosomal protein SA) |
| 360 | 1.1772 | 1.3871 | 1.5238 | 1.5783 | 1.3957 | 2 | UDP-N-acetyl-alpha-D-galactosamine(N-acetylneuraminyl)- |
| | | | | | | | galactosylglucosylceramide-beta-1,4-N-acetylgalactosaminyltransferase |
| 361 | 0.9918 | 1.3959 | 1.7243 | 1.7036 | 1.4070 | 2 | ribosomal protein L3 |
| 362 | 0.9236 | 1.3424 | 1.7120 | 1.7548 | 1.3989 | 2 | fibrillin 1 |
| 363 | 1.0019 | 1.6503 | 1.6219 | 1.8668 | 1.7896 | 2 | Unknown |
| 364 | 0.9236 | 1.5383 | 1.5327 | 1.7055 | 1.6684 | 2 | claudin 4 |
| 365 | 0.8999 | 1.1923 | 1.1938 | 1.2369 | 1.2125 | 2 | E26 avian leukemia oncogene 2,3′ domain |
| 366 | 1.0054 | 1.5161 | 1.4612 | 1.6057 | 1.5306 | 2 | endothelin 1 |
| 367 | 0.9438 | 1.5512 | 1.5688 | 1.5612 | 1.5255 | 2 | tyrosine 3-monooxygenase/tryptophan 5-monooxygenase activation protein, |
| | | | | | | | eta polypeptide |
| 368 | 0.9070 | 1.3337 | 1.3471 | 1.3404 | 1.3515 | 2 | expressed sequence AI586180 |
| 369 | 1.0953 | 3.0749 | 3.0393 | 2.8424 | 2.8680 | 2 | tissue inhibitor of metalloproteinase |
| 370 | 0.9175 | 1.1528 | 1.1523 | 1.1179 | 1.1417 | 2 | SWI/SNF related, matrix associated, actin dependent regulator of chromatin, |
| | | | | | | | subfamily a, member 5 |
| 371 | 1.0293 | 1.2172 | 1.2430 | 1.2255 | 1.2593 | 2 | BCL2-antagonist/killer 1 |
| 372 | 0.9142 | 1.7542 | 1.6654 | 1.7301 | 1.7848 | 2 | annexin A5 |
| 373 | 1.0614 | 1.5743 | 1.5697 | 1.5836 | 1.6713 | 2 | core promoter element binding protein |
| 374 | 0.8819 | 1.6174 | 1.9000 | 1.7364 | 1.4644 | 2 | ribosomal protein S4, X-linked |
| 375 | 1.0486 | 2.0169 | 2.3995 | 2.1620 | 1.9031 | 2 | SH3 domain binding glutamic acid-rich protein-like 3 |
| 376 | 1.1791 | 1.8132 | 1.9389 | 1.9017 | 1.7616 | 2 | CD68 antigen |
| 377 | 0.9477 | 1.2291 | 1.2628 | 1.2923 | 1.1989 | 2 | ubiquitin-conjugating enzyme E2L 3 |
| 378 | 0.9927 | 1.0910 | 1.1150 | 1.0879 | 1.0874 | 2 | Mus musculus, Similar to hypothetical protein FLJ13213, clone MGC: 28555 |
| | | | | | | | IMAGE: 4206928, mRNA, complete cds |
| 379 | 1.0583 | 1.3916 | 1.4379 | 1.3821 | 1.3659 | 2 | DNA segment, Chr 17, ERATO Doi 441, expressed |
| 380 | 0.9295 | 1.8598 | 2.1680 | 1.7429 | 2.0043 | 2 | transforming growth factor, beta induced, 68 kDa |
| 381 | 0.9997 | 1.1814 | 1.2499 | 1.1804 | 1.2053 | 2 | eukaryotic translation initiation factor 4, gamma 2 |
| 382 | 1.0108 | 1.7742 | 2.1777 | 2.6390 | 2.4383 | 2 | lymphocyte antigen 6 complex, locus E |
| 383 | 0.9871 | 1.1141 | 1.1763 | 1.2068 | 1.1977 | 2 | RIKEN cDNA 4921528E07 gene |
| 384 | 0.8993 | 1.3005 | 1.3760 | 1.4886 | 1.4806 | 2 | annexin A6 |
| 385 | 1.0427 | 1.3580 | 1.4405 | 1.4577 | 1.4921 | 2 | ribosomal protein S23 |
| 386 | 1.0454 | 1.2103 | 1.2506 | 1.2689 | 1.2617 | 2 | protein tyrosine phosphatase, non-receptor type 9 |
| 387 | 1.0722 | 1.3211 | 1.3274 | 1.4337 | 1.4424 | 2 | Unknown |
| 388 | 0.9876 | 1.3432 | 1.3314 | 1.4721 | 1.5478 | 2 | eukaryotic translation initiation factor 4A1 |
| 389 | 0.9192 | 1.3767 | 1.4751 | 1.5131 | 1.7564 | 2 | baculoviral IAP repeat-containing 1a |
| 390 | 1.0092 | 1.3138 | 1.4509 | 1.4923 | 1.5790 | 2 | prothymosin alpha |
| 391 | 0.9321 | 1.1637 | 1.3202 | 1.2866 | 1.3767 | 2 | actin related protein ⅔ complex, subunit 3 (21 kDa) |
| 392 | 1.0014 | 1.2887 | 1.5769 | 1.4820 | 1.5893 | 2 | CD53 antigen |
| 393 | 1.0693 | 1.2970 | 1.4700 | 1.3569 | 1.5194 | 2 | Unknown |
| 394 | 0.8751 | 1.1307 | 1.4512 | 1.2749 | 1.4358 | 2 | Mus musculus, Similar to dendritic cell protein, clone MGC: 11741 IMAGE: |
| | | | | | | | 3969335, mRNA, complete cds |
| 395 | 0.9679 | 1.4380 | 1.6503 | 1.5282 | 1.5450 | 2 | hemopoietic cell phosphatase |
| 396 | 1.0612 | 1.2098 | 1.2772 | 1.2516 | 1.2652 | 2 | 2′-5′ oligoadenylate synthetase 1A |
| 397 | 1.0200 | 1.2776 | 1.3701 | 1.3446 | 1.3559 | 2 | DNA segment, Chr 12, ERATO Doi 604, expressed |
| 398 | 1.0539 | 1.7415 | 3.0036 | 2.6951 | 2.6722 | 2 | thymosin, beta 4, X chromosome |
| 399 | 0.9107 | 1.7153 | 2.3956 | 2.3542 | 2.2252 | 2 | small inducible cytokine B subfamily (Cys-X-Cys), member 10 |
| 400 | 0.9790 | 1.3101 | 1.6005 | 1.5099 | 1.5488 | 2 | AXL receptor tyrosine kinase |
| 401 | 0.9711 | 1.4342 | 1.9607 | 1.4200 | 1.8099 | 2 | small inducible cytokine A9 |
| 402 | 1.0407 | 1.1287 | 1.2267 | 1.1205 | 1.1935 | 2 | SAR1a gene homolog (S. cerevisiae) |
| 403 | 1.1581 | 1.4242 | 1.7527 | 1.3748 | 1.6223 | 2 | small inducible cytokine A7 |
| 404 | 1.1344 | 1.2385 | 1.3246 | 1.1894 | 1.2853 | 2 | nestin |
| 405 | 0.9768 | 1.1650 | 1.2693 | 1.0482 | 1.1947 | 2 | Mus musculus, clone MGC: 19361 IMAGE: 4242170, mRNA, complete cds |
| 406 | 1.0085 | 1.1639 | 1.1872 | 1.1016 | 1.1799 | 2 | heparan sulfate 2-O-sulfotransferase 1 |
| 407 | 0.9370 | 1.2726 | 1.4005 | 1.0858 | 1.4017 | 2 | chemokine (C-C) receptor 5 |
| 408 | 0.8979 | 1.1824 | 1.3053 | 1.0655 | 1.3085 | 2 | arginine-rich, mutated in early stage tumors |
| 409 | 0.9791 | 1.1075 | 1.1639 | 1.0499 | 1.1448 | 2 | immunoglobulin superfamily, member 8 |
| 410 | 0.9493 | 1.2617 | 1.2057 | 1.2604 | 1.4009 | 2 | ubiquitin-conjugating enzyme E2N |
| 411 | 1.1199 | 1.3886 | 1.3575 | 1.4669 | 1.5721 | 2 | cell division cycle 42 homolog (S. cerevisiae) |
| 412 | 0.9910 | 1.2370 | 1.2246 | 1.1783 | 1.3402 | 2 | RIKEN cDNA 4930506M07 gene |
| 413 | 1.1674 | 1.3938 | 1.3242 | 1.3312 | 1.4198 | 2 | diaphorase 1 (NADH) |
| 414 | 0.9958 | 1.3622 | 1.2894 | 1.2955 | 1.4732 | 2 | phorbol-12-myristate-13-acetate-induced protein 1 |
| 415 | 1.0976 | 1.2601 | 1.2696 | 1.2100 | 1.3906 | 2 | SET translocation |
| 416 | 0.8633 | 1.1617 | 1.2776 | 1.0506 | 1.4714 | 2 | interleukin 1 receptor, type I |
| 417 | 0.9730 | 1.1367 | 1.2428 | 1.1320 | 1.3682 | 2 | src-like adaptor protein |
| 418 | 1.0087 | 1.3615 | 1.3483 | 1.2931 | 1.3871 | 2 | spermidine/spermine N1-acetyl transferase |
| 419 | 0.9741 | 1.3756 | 1.4229 | 1.3068 | 1.4986 | 2 | small nuclear ribonucleoprotein polypeptide G |
| 420 | 0.9006 | 1.3014 | 1.3478 | 1.2439 | 1.4480 | 2 | CD38 antigen |
| 421 | 0.8681 | 1.5985 | 1.8253 | 1.3655 | 1.9501 | 2 | glycoprotein 49 B |
| 422 | 0.9150 | 1.2345 | 1.2999 | 1.1371 | 1.3884 | 2 | ubiquitin-like 1 (sentrin) activating enzyme E1B |
| 423 | 1.2153 | 2.1360 | 2.5678 | 2.1215 | 2.6374 | 2 | small inducible cytokine A2 |
| 424 | 1.0132 | 1.1831 | 1.2480 | 1.1615 | 1.2605 | 2 | expressed sequence AA589392 |
| 425 | 0.9345 | 0.6618 | 0.8351 | 0.7595 | 0.6684 | 3 | Mus musculus adult male tongue cDNA, RIKEN full-length enriched library, |
| | | | | | | | clone:2310065B16:erythrocyte protein band 4.1, full insert sequence |
| 426 | 0.8801 | 0.6281 | 0.7446 | 0.8023 | 0.6418 | 3 | peroxisomal delta3, delta2-enoyl-Coenzyme A isomerase |
| 427 | 0.8391 | 0.4198 | 0.6520 | 0.6794 | 0.4942 | 3 | solute carrier family 27 (fatty acid transporter), member 2 |
| 428 | 0.9140 | 0.3439 | 0.5362 | 0.6193 | 0.5699 | 3 | expressed sequence AI159688 |
| 429 | 1.1527 | 0.5483 | 0.7967 | 0.9056 | 0.9320 | 3 | Unknown |
| 430 | 1.0530 | 0.6802 | 0.8739 | 0.9264 | 0.9410 | 3 | RIKEN cDNA 2410029D23 gene |
| 431 | 0.8908 | 0.4317 | 0.5903 | 0.6253 | 0.7110 | 3 | proteaseome (prosome, macropain) 28 subunit, 3 |
| 432 | 1.1135 | 0.3503 | 0.6078 | 0.6820 | 0.6861 | 3 | poly (A) polymerase alpha |
| 433 | 1.0378 | 0.6428 | 0.8193 | 0.8424 | 0.8880 | 3 | estrogen related receptor, alpha |
| 434 | 0.7955 | 0.3840 | 0.5353 | 0.5684 | 0.6072 | 3 | solute carrier family 22 (organic cation transporter), member 5 |
| 435 | 0.9197 | 0.6583 | 0.7685 | 0.7898 | 0.8061 | 3 | mitsugumin 29 |
| 436 | 0.8775 | 0.3124 | 0.5760 | 0.7141 | 0.5975 | 3 | Mus musculus, Similar to hypothetical protein FLJ21634, clone MGC: 19374 |
| | | | | | | | IMAGE: 2631696, mRNA, complete cds |
| 437 | 0.8205 | 0.5285 | 0.6892 | 0.7204 | 0.6726 | 3 | oxysterol binding protein-like 1A |
| 438 | 1.0303 | 0.5004 | 0.7618 | 0.7590 | 0.7135 | 3 | glutathione S-transferase, theta 2 |
| 439 | 0.9297 | 0.5420 | 0.7451 | 0.7493 | 0.7508 | 3 | peroxisomal sarcosine oxidase |
| 440 | 0.7442 | 0.4601 | 0.6381 | 0.6326 | 0.6378 | 3 | coproporphyrinogen oxidase |
| 441 | 0.7089 | 0.3552 | 0.5346 | 0.4980 | 0.6839 | 3 | glycerol kinase |
| 442 | 0.8985 | 0.1439 | 0.4028 | 0.4404 | 0.6333 | 3 | solute carrier family 12, member 1 |
| 443 | 1.0339 | 0.6248 | 0.7932 | 0.8344 | 0.9307 | 3 | Blu protein |
| 444 | 0.7819 | 0.3947 | 0.5601 | 0.5682 | 0.6998 | 3 | hydroxysteroid dehydrogenase-1, delta<5>-3-beta |
| 445 | 0.9535 | 0.4577 | 0.5970 | 0.8027 | 0.8859 | 3 | fibulin 5 |
| 446 | 1.0207 | 0.7390 | 0.8378 | 0.9178 | 0.9822 | 3 | reticulon 3 |
| 447 | 0.9986 | 0.7013 | 0.8551 | 0.9231 | 1.0109 | 3 | UDP-Gal:betaGlcNAc beta 1,3-galactosyltransferase, polypeptide 3 |
| 448 | 0.9856 | 0.4209 | 0.7348 | 0.9121 | 1.0071 | 3 | selenoprotein P, plasma, 1 |
| 449 | 1.0329 | 0.5009 | 0.7450 | 0.9372 | 1.0031 | 3 | Mus musculus, clone IMAGE: 3589087, mRNA, partial cds |
| 450 | 0.9919 | 0.7406 | 0.8638 | 0.9475 | 0.9708 | 3 | ESTs |
| 451 | 1.0324 | 0.8305 | 0.9196 | 1.0912 | 0.9137 | 3 | 5-azacytidine induced gene 1 |
| 452 | 0.9771 | 0.6699 | 0.8176 | 1.0389 | 0.7884 | 3 | alkaline phosphatase 2, liver |
| 453 | 0.8509 | 0.4999 | 0.5656 | 0.9052 | 0.6255 | 3 | insulin-like growth factor binding protein 4 |
| 454 | 0.9063 | 0.7210 | 0.7536 | 0.9344 | 0.8102 | 3 | neuronal guanine nucleotide exchange factor |
| 455 | 1.0176 | 0.7055 | 0.7414 | 0.9316 | 0.7359 | 3 | EST AI181838 |
| 456 | 0.7568 | 0.6311 | 0.6312 | 0.7282 | 0.6289 | 3 | nuclear receptor coactivator 4 |
| 457 | 1.0770 | 0.7827 | 0.8552 | 0.9984 | 0.8107 | 3 | RIKEN cDNA 1110002C08 gene |
| 458 | 1.1253 | 0.5364 | 0.6409 | 0.8571 | 0.6057 | 3 | RIKEN cDNA 1200011D11 gene |
| 459 | 1.0182 | 0.8019 | 0.8445 | 0.9453 | 0.8437 | 3 | expressed sequence AI480660 |
| 460 | 1.0078 | 0.5996 | 0.6854 | 0.8644 | 0.7061 | 3 | heat-responsive protein 12 |
| 461 | 1.0362 | 0.8239 | 0.8753 | 0.9706 | 0.8768 | 3 | succinate-Coenzyme A ligase, GDP-forming, beta subunit |
| 462 | 0.9929 | 0.5315 | 0.6021 | 0.7369 | 0.6669 | 3 | elastase 1, pancreatic |
| 463 | 1.0401 | 0.7373 | 0.8051 | 0.9084 | 0.8444 | 3 | RIKEN cDNA 3010027G13 gene |
| 464 | 0.8619 | 0.5529 | 0.6297 | 0.7155 | 0.6589 | 3 | glutathione transferase zeta 1 (maleylacetoacetate isomerase) |
| 465 | 0.8571 | 0.4509 | 0.5741 | 0.6150 | 0.5582 | 3 | RIKEN cDNA 0610011L04 gene |
| 466 | 1.1680 | 0.7192 | 0.8663 | 0.9687 | 0.8414 | 3 | cytochrome c oxidase, subunit VIIa 3 |
| 467 | 0.9737 | 0.5262 | 0.7046 | 0.8461 | 0.7037 | 3 | expressed sequence AI835705 |
| 468 | 1.0104 | 0.5872 | 0.7190 | 0.8710 | 0.7932 | 3 | brain protein 44-like |
| 469 | 1.1337 | 0.5399 | 0.7227 | 0.9804 | 0.7850 | 3 | RIKEN cDNA 1810013B01 gene |
| 470 | 1.0571 | 0.6297 | 0.7817 | 0.9716 | 0.8641 | 3 | phenylalkylamine Ca2+ antagonist (emopamil) binding protein |
| 471 | 1.1129 | 0.6919 | 0.8198 | 1.0341 | 0.8359 | 3 | ribonucleotide reductase M1 |
| 472 | 0.8054 | 0.5784 | 0.6332 | 0.7565 | 0.6853 | 3 | FK506 binding protein 12-rapamycin associated protein 1 |
| 473 | 1.1953 | 0.7803 | 0.8850 | 1.0894 | 0.9492 | 3 | RIKEN cDNA 0610006N12 gene |
| 474 | 1.0970 | 0.6433 | 0.7445 | 1.0358 | 0.8419 | 3 | RIKEN cDNA 1810054O13 gene |
| 475 | 0.8446 | 0.5412 | 0.6005 | 0.8320 | 0.6665 | 3 | RIKEN cDNA 2310051E17 gene |
| 476 | 1.1011 | 1.3217 | 1.3461 | 1.1891 | 1.0091 | 4 | mitogene activated protein kinase 13 |
| 477 | 1.1221 | 1.3644 | 1.4586 | 1.2110 | 1.0056 | 4 | DNA primase, p49 subunit |
| 478 | 1.0254 | 1.2717 | 1.3756 | 1.1513 | 0.9416 | 4 | chitinase 3-like 3 |
| 479 | 1.1328 | 1.4784 | 1.7963 | 1.4788 | 0.9639 | 4 | ribosomal protein L28 |
| 480 | 1.1227 | 1.3555 | 1.4238 | 1.2682 | 0.9761 | 4 | Mus musculus, Similar to hypothetical protein MGC3133, clone MGC: 11596 |
| | | | | | | | IMAGE: 3965951, mRNA, complete cds |
| 481 | 1.0459 | 1.1818 | 1.2658 | 1.1469 | 0.9606 | 4 | ubiquitin-like 1 (sentrin) activating enzyme E1A |
| 482 | 1.0698 | 1.1602 | 1.2053 | 1.1283 | 1.0173 | 4 | expressed sequence AI448212 |
| 483 | 1.0287 | 1.1156 | 1.2016 | 1.1337 | 0.9083 | 4 | Mus musculus, clone MGC: 6377 IMAGE: 3499365, mRNA, complete cds |
| 484 | 1.0928 | 1.1820 | 1.2455 | 1.1398 | 0.9300 | 4 | RIKEN cDNA 2610511O17 gene |
| 485 | 1.0948 | 1.2156 | 1.2584 | 1.1247 | 0.9444 | 4 | RIKEN cDNA 1110020L19 gene |
| 486 | 0.9522 | 1.2425 | 1.1062 | 1.0803 | 0.8774 | 4 | retinoic acid induced 1 |
| 487 | 1.0865 | 1.4441 | 1.2235 | 1.1786 | 0.9942 | 4 | RIKEN cDNA 1810023B24 gene |
| 488 | 0.9952 | 1.2036 | 1.1622 | 1.1417 | 0.8479 | 4 | hepatoma-derived growth factor |
| 489 | 1.0214 | 1.1893 | 1.1494 | 1.1261 | 0.9821 | 4 | steroid receptor RNA activator 1 |
| 490 | 0.9646 | 1.1555 | 1.1351 | 1.0714 | 0.9045 | 4 | schlafen 4 |
| 491 | 1.2059 | 1.2836 | 1.5301 | 1.5339 | 1.0903 | 4 | lactate dehydrogenase 1, A chain |
| 492 | 1.1800 | 1.2568 | 1.3462 | 1.3466 | 1.1501 | 4 | Mus musculus, clone IMAGE: 4456744, mRNA, partial cds |
| 493 | 1.1552 | 1.2438 | 1.3568 | 1.2886 | 1.1167 | 4 | regulator of G-protein signaling 19 interacting protein 1 |
| 494 | 1.0002 | 1.0878 | 1.2716 | 1.2027 | 0.8901 | 4 | guanosine diphosphate (GDP) dissociation inhibitor 3 |
| 495 | 0.9314 | 1.1888 | 1.4098 | 1.5523 | 0.9862 | 4 | dolichyl-di-phosphooligosaccharide-protein glycotransferase |
| 496 | 0.9355 | 1.1848 | 1.4317 | 1.4925 | 1.0033 | 4 | procollagen, type V, alpha 1 |
| 497 | 1.1546 | 1.4761 | 1.6092 | 1.5930 | 1.1651 | 4 | ribosomal protein L8 |
| 498 | 0.9680 | 1.1317 | 1.2124 | 1.1458 | 1.0059 | 4 | peptidylprolyl isomerase (cyclophilin)-like 1 |
| 499 | 1.0720 | 1.6647 | 2.0127 | 1.6687 | 1.1292 | 4 | acidic ribosomal phosphoprotein PO |
| 500 | 1.1094 | 1.7959 | 2.0748 | 1.7960 | 1.1524 | 4 | ribosomal protein S2 |
| 501 | 1.0087 | 1.8326 | 2.1030 | 2.1386 | 1.3589 | 4 | ribosomal protein L10A |
| 502 | 0.9881 | 1.6212 | 1.9293 | 1.8679 | 1.2267 | 4 | ribosomal protein L19 |
| 503 | 1.0133 | 1.7517 | 2.4000 | 2.5281 | 1.3040 | 4 | RIKEN cDNA 1810009M0I gene |
| 504 | 1.0884 | 1.8047 | 2.2731 | 2.2732 | 1.3183 | 4 | ribosomal protein, large, P1 |
| 505 | 1.0083 | 1.1548 | 1.2145 | 1.2296 | 1.0605 | 4 | expressed sequence C86302 |
| 506 | 1.1156 | 1.8020 | 2.2258 | 1.7922 | 1.3432 | 4 | ribosomal protein S16 |
| 507 | 1.0772 | 1.5091 | 1.6772 | 1.4961 | 1.2653 | 4 | Mus musculus, basic transcription factor 3, clone MGC: 6799 IMAGE: |
| | | | | | | | 2648048, mRNA, complete cds |
| 508 | 1.0513 | 1.7752 | 2.2322 | 1.9914 | 1.2727 | 4 | cathepsin D |
| 509 | 1.0558 | 1.7314 | 2.0719 | 1.9098 | 1.3518 | 4 | ribosomal protein S7 |
| 510 | 1.0319 | 1.4863 | 1.7487 | 1.6553 | 1.2705 | 4 | RIKEN cDNA 0610025G13 gene |
| 511 | 1.0301 | 1.6749 | 2.0161 | 1.7487 | 1.3056 | 4 | tropomyosin 2, beta |
| 512 | 0.9851 | 1.4157 | 1.6599 | 1.5067 | 1.2141 | 4 | ribosomal protein S15 |
| 513 | 0.9221 | 0.8267 | 0.7881 | 1.1023 | 1.2316 | 5 | RIKEN cDNA 3010001A07 gene |
| 514 | 0.9981 | 0.9938 | 0.8378 | 1.3772 | 1.6072 | 5 | AE binding protein 1 |
| 515 | 1.0544 | 1.0451 | 0.9600 | 1.3268 | 1.3683 | 5 | nuclear receptor subfamily 2, group F, member 2 |
| 516 | 1.0441 | 1.0086 | 0.9497 | 1.2215 | 1.2676 | 5 | nucleolar protein GU2 |
| 517 | 1.0677 | 1.0196 | 1.0196 | 1.2801 | 1.2882 | 5 | RIKEN cDNA 1700016A15 gene |
| 518 | 1.1450 | 1.0074 | 1.0750 | 1.6170 | 1.6983 | 5 | protein tyrosine phosphatase, receptor type, C polypeptide-associated protein |
| 519 | 1.0490 | 0.9791 | 1.0259 | 1.3848 | 1.3794 | 5 | expressed sequence C80611 |
| 520 | 1.1572 | 1.0877 | 1.0899 | 1.3343 | 1.2886 | 5 | expressed sequence C85317 |
| 521 | 1.0744 | 1.0001 | 1.0402 | 1.2923 | 1.2529 | 5 | protein tyrosine phosphatase receptor type, O |
| 522 | 1.0688 | 0.9723 | 1.0203 | 1.3206 | 1.2484 | 5 | bone morphogenetic protein receptor, type 1A |
| 523 | 1.1004 | 0.9990 | 1.0658 | 1.2307 | 1.2252 | 5 | RIKEN cDNA 2610302I02 gene |
| 524 | 0.8396 | 0.7401 | 0.7912 | 0.9653 | 0.9894 | 5 | src homology 2 domain-containing transforming protein D |
| 525 | 1.0580 | 0.9098 | 1.0042 | 1.3665 | 1.4267 | 5 | transcription factor 4 |
| 526 | 0.8687 | 0.8022 | 0.7949 | 0.9701 | 0.9744 | 5 | ESTs |
| 527 | 0.9766 | 0.8264 | 0.8621 | 1.1258 | 1.1708 | 5 | peptidylprolyl isomerase C |
| 528 | 1.1335 | 0.9919 | 1.0401 | 1.3515 | 1.4512 | 5 | RIKEN cDNA 3110001N18 gene |
| 529 | 0.8920 | 0.7754 | 0.7748 | 1.0905 | 1.1534 | 5 | speckle-type POZ protein |
| 530 | 1.0497 | 0.9373 | 0.9611 | 1.2325 | 1.2627 | 5 | ESTs, Weakly similar to simple repeat sequence-containing |
| | | | | | | | transcript (Mus musculus) (M. musculus) |
| 531 | 1.1195 | 0.8571 | 1.2821 | 1.6795 | 1.8423 | 5 | transcription factor 21 |
| 532 | 1.1442 | 0.9930 | 1.3094 | 1.6671 | 1.7597 | 5 | macrophage scavenger receptor 2 |
| 533 | 1.1838 | 1.0801 | 1.2406 | 1.2964 | 1.4212 | 5 | ras homolog D (RhoD) |
| 534 | 0.9662 | 0.9097 | 1.1485 | 1.2346 | 1.4239 | 5 | ESTs |
| 535 | 1.2090 | 1.1308 | 1.3565 | 1.4311 | 1.5207 | 5 | toll-like receptor 2 |
| 536 | 0.9952 | 0.8051 | 0.9644 | 1.6714 | 2.4657 | 5 | RIKEN cDNA 1110032A13 gene |
| 537 | 0.9638 | 0.8947 | 0.9198 | 1.1363 | 1.2490 | 5 | expressed sequence AI848691 |
| 538 | 0.9554 | 0.8621 | 0.9194 | 1.1748 | 1.3264 | 5 | ESTs, Weakly similar to TS13 MOUSE TESTIS-SPECIFIC PROTEIN |
| | | | | | | | PBS13 (M. musculus) |
| 539 | 1.0082 | 0.9228 | 0.9640 | 1.1534 | 1.2696 | 5 | DNA segment, Chr 8, Brigham & Women's Genetics 1112 expressed |
| 540 | 1.0235 | 0.9920 | 0.9787 | 1.1733 | 1.3926 | 5 | activity-dependent neuroprotective protein |
| 541 | 1.1077 | 1.0587 | 1.0953 | 1.6039 | 2.3854 | 5 | matrix metalloproteinase 7 |
| 542 | 1.1479 | 0.9773 | 1.0504 | 1.7190 | 2.5428 | 5 | expressed sequence AI194696 |
| 543 | 0.9860 | 0.8914 | 0.9622 | 1.4171 | 2.0505 | 5 | retinoic acid early transcript gamma |
| 544 | 0.7507 | 0.6726 | 0.8611 | 1.7079 | 2.9941 | 5 | complement factor H related protein 3A4/5G4 |
| 545 | 1.0361 | 1.0285 | 1.1443 | 1.3669 | 1.6479 | 5 | early development regulator 2 (homolog of polyhomeotic 2) |
| 546 | 0.9563 | 0.8374 | 1.0064 | 1.1918 | 1.3697 | 5 | gamma-glutamyl hydrolase |
| 547 | 0.8903 | 0.7658 | 1.0432 | 1.4121 | 1.8760 | 5 | decorin |
| 548 | 1.0382 | 0.9776 | 1.0743 | 1.1949 | 1.3286 | 5 | myocyte enhancer factor 2A |
| 549 | 1.0094 | 0.5922 | 1.0062 | 3.3025 | 5.1497 | 5 | histocompatibility 2, class II antigen A, alpha |
| 550 | 0.9496 | 0.7367 | 1.0097 | 2.1319 | 2.8584 | 5 | complement component factor h |
| 551 | 1.1506 | 0.8278 | 1.2558 | 2.4083 | 3.8563 | 5 | histocompatibility 2, class II antigen E beta |
| 552 | 1.0345 | 0.9905 | 1.0673 | 1.2226 | 1.3108 | 5 | ganglioside-induced differentiation-associated-protein 3 |
| 553 | 1.0058 | 0.9940 | 1.2866 | 1.3443 | 1.8569 | 5 | interferon activated gene 204 |
| 554 | 1.0558 | 0.9892 | 1.1895 | 1.1994 | 1.5192 | 5 | ESTs, Weakly similar to 2022314A granule cell marker protein (M. musculus) |
| 555 | 0.9533 | 1.0053 | 1.1020 | 1.2514 | 1.6942 | 5 | integrin-associated protein |
| 556 | 1.0788 | 1.0886 | 1.1943 | 1.2789 | 1.4841 | 5 | RIKEN cDNA 2310046G15 gene |
| 557 | 1.0682 | 1.0637 | 1.1649 | 1.2524 | 1.3753 | 5 | RIKEN cDNA E130113K08 gene |
| 558 | 1.0759 | 1.1409 | 1.3359 | 1.6449 | 2.1164 | 5 | CD48 antigen |
| 559 | 0.9055 | 0.9716 | 1.2024 | 1.4363 | 1.8141 | 5 | serine protease inhibitor 6 |
| 560 | 1.0835 | 1.1251 | 1.1875 | 1.4436 | 1.2944 | 5 | ubiquitin-conjugating enzyme E2D 2 |
| 561 | 0.9050 | 0.9775 | 1.1514 | 1.7313 | 1.3618 | 5 | RAS-related C3 botulinum substrate 2 |
| 562 | 0.9589 | 0.8678 | 1.3958 | 2.8748 | 1.8466 | 5 | glypican 3 |
| 563 | 1.0452 | 1.0441 | 1.1399 | 1.2753 | 1.1817 | 5 | Mus musculus, Similar to hypothetical protein FLJ20245, clone MGC: 7940 |
| | | | | | | | IMAGE: 3584061, mRNA, complete cds |
| 564 | 1.0777 | 1.0600 | 1.1755 | 1.3873 | 1.2101 | 5 | expressed sequence AU042434 |
| 565 | 1.0284 | 1.0269 | 1.2169 | 1.6528 | 1.3402 | 5 | benzodiazepine receptor, peripheral |
| 566 | 1.1138 | 1.1173 | 1.1857 | 1.3590 | 1.2334 | 5 | RIKEN cDNA 3321401G04 gene |
| 567 | 1.0393 | 0.9358 | 1.0422 | 1.3203 | 1.1945 | 5 | hemochromatosis |
| 568 | 1.2057 | 1.1632 | 1.2238 | 1.3369 | 1.2510 | 5 | RIKEN cDNA 1810043O07 gene |
| 569 | 1.0767 | 0.9953 | 1.1008 | 1.4273 | 1.2152 | 5 | expressed sequence AI451355 |
| 570 | 0.7786 | 0.8853 | 1.2704 | 1.6580 | 1.8390 | 5 | mannose receptor, C type 1 |
| 571 | 0.8371 | 0.8513 | 1.1095 | 1.3446 | 1.5130 | 5 | calcium channel, voltage-dependent, beta 3 subunit |
| 572 | 1.0800 | 1.2170 | 1.7844 | 2.5241 | 3.1068 | 5 | macrophage expressed gene 1 |
| 573 | 0.7878 | 0.9131 | 1.2493 | 1.8788 | 2.2251 | 5 | T-cell specific GTPase |
| 574 | 0.8758 | 0.9908 | 1.0771 | 1.2393 | 1.2927 | 5 | centrin 3 |
| 575 | 1.0187 | 1.1495 | 1.3851 | 2.1191 | 2.0841 | 5 | lysosomal-associated protein transmembrane 5 |
| 576 | 0.9398 | 1.0141 | 1.1014 | 1.3287 | 1.3207 | 5 | chloride channel calcium activated 1 |
| 577 | 1.0142 | 1.2939 | 2.1261 | 4.4031 | 4.5859 | 5 | cathepsin S |
| 578 | 0.9640 | 1.0862 | 1.2569 | 1.5891 | 1.5971 | 5 | protein tyrosine phosphatase, receptor type, C |
| 579 | 1.0523 | 1.1920 | 1.2192 | 1.3611 | 1.5243 | 5 | expressed sequence AI604920 |
| 580 | 0.9848 | 1.1392 | 1.1614 | 1.3111 | 1.4113 | 5 | runt related transcription factor 1 |
| 581 | 0.9640 | 1.2690 | 1.3699 | 1.9377 | 2.2444 | 5 | oncostatin receptor |
| 582 | 0.9036 | 1.0784 | 1.0787 | 1.3259 | 1.4879 | 5 | neuropilin |
| 583 | 0.9313 | 1.1539 | 1.3170 | 2.1477 | 3.3642 | 5 | CD52 antigen |
| 584 | 1.0126 | 1.1442 | 1.2098 | 1.6038 | 2.0581 | 5 | histocompatibility 2, class II, locus DMa |
| 585 | 0.9198 | 0.9953 | 1.1206 | 1.3312 | 1.5158 | 5 | ESTs, Moderately similar to T46312 hypothetical protein DKFZp434J1111.1 |
| | | | | | | | (H. sapiens) |
| 586 | 0.9171 | 1.0215 | 1.0601 | 1.3413 | 1.4274 | 5 | tetratricopeptide repeat domain |
| 587 | 0.9802 | 1.1050 | 1.2201 | 1.6447 | 1.7933 | 5 | protein S (alpha) |
| 588 | 0.9717 | 1.0447 | 1.0976 | 1.2986 | 1.3751 | 5 | Mus musculus, clone MGC: 12159 IMAGE: 3711169, mRNA, complete cds |
| 589 | 0.9930 | 1.0020 | 1.1215 | 1.2755 | 1.2960 | 5 | expressed sequence AI413331 |
| 590 | 1.0306 | 1.0103 | 1.3077 | 1.9098 | 1.7718 | 5 | myristoylated alanine rich protein kinase C substrate |
| 591 | 0.9630 | 0.9591 | 1.3556 | 2.0306 | 1.8587 | 5 | RIKEN cDNA 2410026K10 gene |
| 592 | 1.0140 | 1.0064 | 1.2061 | 1.4592 | 1.4295 | 5 | microfibrillar associated protein 5 |
| 593 | 1.0032 | 0.9118 | 1.1683 | 1.6409 | 1.4837 | 5 | matrix metalloproteinase 2 |
| 594 | 1.0696 | 1.0149 | 1.1799 | 1.4794 | 1.3720 | 5 | RIKEN cDNA 2810418N01 gene |
| 595 | 1.0701 | 0.9878 | 1.3489 | 1.8957 | 1.8346 | 5 | Mus musculus, Similar to DKFZP586B0621 protein, clone MGC: 38635 |
| | | | | | | | IMAGE: 5355789, mRNA, complete cds |
| 596 | 1.1047 | 0.8042 | 1.7386 | 4.4517 | 4.2955 | 5 | Ia-associated invariant chain |
| 597 | 0.8360 | 0.9664 | 1.0969 | 1.6065 | 1.4526 | 5 | nidogen 1 |
| 598 | 0.7294 | 0.9189 | 1.1719 | 2.2828 | 1.8126 | 5 | matrix metalloproteinase 14 (membrane-inserted) |
| 599 | 1.0682 | 1.1253 | 1.2076 | 1.4741 | 1.3753 | 5 | RIKEN cDNA 2610200M23 gene |
| 600 | 0.9714 | 1.1162 | 1.4890 | 2.6282 | 2.1815 | 5 | expressed sequence AI132321 |
| 601 | 1.0294 | 1.1744 | 1.4273 | 2.1617 | 1.8326 | 5 | lymphocyte specific 1 |
| 602 | 1.0111 | 1.0553 | 3.2839 | 7.7740 | 5.5050 | 5 | matrix gamma-carboxyglutamate (gla) protein |
| 603 | 1.0601 | 1.0570 | 1.2026 | 1.3465 | 1.2764 | 5 | Fas apoptotic inhibitory molecule |
| 604 | 1.0292 | 1.2822 | 2.0305 | 3.1921 | 3.0027 | 5 | amiloride binding protein 1 (amine oxidase, copper-containing) |
| 605 | 1.0774 | 1.1961 | 1.9460 | 3.2828 | 2.8276 | 5 | RIKEN cDNA 3021401A05 gene |
| 606 | 0.9645 | 0.8830 | 0.9929 | 1.3430 | 1.2604 | 5 | laminin, alpha 2 |
| 607 | 1.1142 | 1.0543 | 1.1180 | 1.2988 | 1.2559 | 5 | RIKEN cDNA 2310022K15 gene |
| 608 | 1.1579 | 0.9502 | 1.2503 | 1.7561 | 1.7967 | 5 | cystatin C |
| 609 | 1.0163 | 0.9402 | 1.0328 | 1.2297 | 1.2130 | 5 | expressed sequence AI843960 |
| 610 | 1.0341 | 0.9362 | 1.0538 | 1.2459 | 1.2236 | 5 | sulfotransferase-related protein SULT-X1 |
| 611 | 1.1487 | 1.1234 | 1.3384 | 1.9175 | 2.3082 | 5 | EGF-like module containing, mucin-like, hormone receptor-like sequence 1 |
| 612 | 1.0326 | 1.0690 | 1.1895 | 1.5144 | 1.7217 | 5 | apolipoprotein B editing complex 1 |
| 613 | 1.1007 | 1.1309 | 1.5867 | 2.9748 | 3.5097 | 5 | vascular cell adhesion molecule 1 |
| 614 | 1.1983 | 1.1220 | 1.3545 | 1.9983 | 2.1804 | 5 | expressed sequence AW743884 |
| 615 | 1.0716 | 1.0252 | 1.2573 | 1.8115 | 1.8775 | 5 | proteosome (prosome, macropain) subunit, beta type 8 (large |
| | | | | | | | multifunctional protease 7) |
| 616 | 1.0003 | 0.9941 | 1.0611 | 1.5084 | 1.4066 | 5 | papillary renal cell carcinoma (translocation-associated) |
| 617 | 1.0292 | 1.0219 | 1.0399 | 1.2878 | 1.2662 | 5 | ESTs |
| 618 | 1.0690 | 1.0411 | 1.1613 | 1.7251 | 1.7845 | 5 | chemokine orphan receptor 1 |
| 619 | 1.1305 | 1.0553 | 1.2562 | 2.3534 | 2.4045 | 5 | serine (or cysteine) proteinase inhibitor, clade G (C1 inhibitor), member 1 |
| 620 | 1.0690 | 0.9488 | 1.5631 | 4.9592 | 4.3560 | 5 | Unknown |
| 621 | 1.0132 | 0.9879 | 1.0620 | 1.3125 | 1.2872 | 5 | ESTs |
| 622 | 0.9379 | 1.0466 | 1.1406 | 1.8888 | 1.9354 | 5 | RIKEN cDNA 2700038M07 gene |
| 623 | 1.0088 | 1.0616 | 1.1703 | 1.7674 | 1.8580 | 5 | serine (or cysteine) proteinase inhibitor, clade E (nexin, plasminogen activator |
| | | | | | | | inhibitor type 1), member 2 |
| 624 | 1.0431 | 1.1275 | 1.3204 | 2.1770 | 2.0270 | 5 | Mus musculus, Similar to unc93 (C. elegans) homolog B, clone MGC: 25627 |
| | | | | | | | IMAGE: 4209296, mRNA, complete cds |
| 625 | 0.9776 | 0.9898 | 1.0467 | 1.2738 | 1.2766 | 5 | cytidine 5′-triphosphate synthase 2 |
| 626 | 0.9918 | 1.0013 | 1.1452 | 1.6077 | 1.5515 | 5 | Mus musculus, clone MGC: 38363 IMAGE: 5344986, mRNA, complete cds |
| 627 | 0.7974 | 0.8055 | 1.0105 | 1.7275 | 1.6969 | 5 | apolipoprotein E |
| 628 | 0.9722 | 1.2339 | 1.0575 | 1.7851 | 1.5579 | 5 | solute carrier family 34 (sodium phosphate), member 2 |
| 629 | 1.0529 | 1.2319 | 1.1334 | 1.4900 | 1.3973 | 5 | NCK-associated protein 1 |
| 630 | 0.9233 | 1.0810 | 0.9506 | 1.3671 | 1.2054 | 5 | max binding protein |
| 631 | 1.0486 | 1.3466 | 1.0930 | 1.7340 | 1.5690 | 5 | platelet derived growth factor, B polypeptide |
| 632 | 1.1209 | 1.3064 | 1.1529 | 1.5690 | 1.4581 | 5 | expressed sequence AA408783 |
| 633 | 0.9676 | 1.1340 | 1.0857 | 1.4115 | 1.3635 | 5 | Mus musculus, Similar to nucleolar cysteine-rich protein, clone MGC: 6718 |
| | | | | | | | IMAGE: 3586161, mRNA, complete cds |
| 634 | 1.0822 | 1.1773 | 1.1551 | 1.3483 | 1.3255 | 5 | non-catalytic region of tyrosine kinase adaptor protein 1 |
| 635 | 0.9486 | 1.0770 | 1.0557 | 1.3062 | 1.3189 | 5 | ring finger protein (C3HC4 type) 19 |
| 636 | 1.0654 | 1.1699 | 1.1703 | 1.3650 | 1.3592 | 5 | spectrin SH3 domain binding protein 1 |
| 637 | 1.0663 | 1.1543 | 1.1307 | 1.5507 | 1.5017 | 5 | Unknown |
| 638 | 0.9880 | 1.0673 | 1.0618 | 1.3613 | 1.2816 | 5 | protein kinase C, delta |
| 639 | 0.9882 | 1.1152 | 1.1118 | 1.4444 | 1.3711 | 5 | nuclear factor of kappa light chain gene enhancer in B-cells 1, p105 |
| 640 | 0.8215 | 0.9917 | 1.0560 | 1.6304 | 1.5544 | 5 | ESTs |
| 641 | 0.7657 | 0.9173 | 0.9616 | 1.5524 | 1.4394 | 5 | X (inactive)-specific transcript, antisense |
| 642 | 0.9198 | 0.9739 | 0.9917 | 1.1951 | 1.1507 | 5 | RIKEN cDNA 4932442K08 gene |
| 643 | 0.9518 | 1.0226 | 0.9973 | 1.5954 | 1.3166 | 5 | platelet-activating factor acetylhydrolase, isoform 1b, alpha1 subunit |
| 644 | 0.9442 | 0.9799 | 0.9990 | 1.4005 | 1.2420 | 5 | mannose-6-phosphate receptor, cation dependent |
| 645 | 1.0084 | 1.1091 | 1.1022 | 1.5706 | 1.3606 | 5 | RIKEN cDNA 5630401J11 gene |
| 646 | 0.9573 | 1.0076 | 1.0124 | 1.2777 | 1.1699 | 5 | RIKEN cDNA 1110007F23 gene |
| 647 | 1.1685 | 1.1799 | 1.1442 | 1.6088 | 1.4724 | 5 | LIM and SH3 protein 1 |
| 648 | 0.9359 | 0.9627 | 0.9283 | 1.3962 | 1.2895 | 5 | casein kinase 1, epsilon |
| 649 | 1.0970 | 1.1310 | 1.0875 | 1.3903 | 1.2864 | 5 | slit homolog 3 (Drosophila) |
| 650 | 1.0915 | 1.1491 | 1.1002 | 1.4888 | 1.3856 | 5 | myeloid differentiation primary response gene 88 |
| 651 | 0.9043 | 0.9824 | 0.9356 | 1.3423 | 1.2115 | 5 | soc-2 (suppressor of clear) homolog (C. elegans) |
| 652 | 0.9322 | 0.9731 | 0.9709 | 1.3387 | 1.3894 | 5 | expressed sequence AI447451 |
| 653 | 0.9735 | 1.0119 | 1.0127 | 1.3834 | 1.3779 | 5 | small inducible cytokine B subfamily, member 5 |
| 654 | 1.1007 | 1.1386 | 1.0671 | 1.7571 | 1.7613 | 5 | Mus musculus, Similar to hypothetical protein FLJ20234, clone MGC: 37525 |
| | | | | | | | IMAGE: 4986113, mRNA, complete cds |
| 655 | 0.9826 | 0.9894 | 0.9796 | 1.1840 | 1.2036 | 5 | expressed sequence C80913 |
| 656 | 1.0175 | 1.1162 | 1.0893 | 1.3116 | 1.3979 | 5 | RIKEN cDNA 1110008B24 gene |
| 657 | 1.0337 | 1.1857 | 1.1148 | 1.7007 | 1.8476 | 5 | CD2-associated protein |
| 658 | 1.0121 | 1.1136 | 1.0550 | 1.3596 | 1.3888 | 5 | growth differentiation factor 8 |
| 659 | 0.9736 | 0.9996 | 0.9385 | 1.3152 | 1.4688 | 5 | trinucleotide repeat containing 11 (THR-associated protein 230 kDa subunit) |
| 660 | 1.1661 | 1.3062 | 1.2799 | 2.0125 | 2.4277 | 5 | Mus musculus, clone IMAGE: 4952483, mRNA, partial cds |
| 661 | 0.9625 | 1.0244 | 1.0178 | 1.3779 | 1.6105 | 5 | baculoviral IAP repeat-containing 3 |
| 662 | 1.1302 | 1.1629 | 1.1327 | 1.2046 | 1.2408 | 5 | expressed sequence AW493404 |
| 663 | 0.9360 | 1.2409 | 1.1174 | 1.4825 | 1.6490 | 5 | Unknown |
| 664 | 0.9137 | 1.0901 | 1.0220 | 1.2927 | 1.3836 | 5 | v-ral simian leukemia viral oncogene homolog A (ras related) |
| 665 | 1.0262 | 1.1734 | 1.1469 | 1.3032 | 1.5956 | 5 | RIKEN cDNA 9130011J04 gene |
| 666 | 1.0714 | 1.3009 | 1.2859 | 1.4240 | 1.9323 | 5 | SFFV proviral integration 1 |
| 667 | 1.0738 | 1.2333 | 1.4036 | 1.3765 | 1.7536 | 5 | CD72 antigen |
| 668 | 1.0207 | 1.1500 | 1.2085 | 1.2554 | 1.5021 | 5 | expressed sequence AI314027 |
| 669 | 0.9480 | 1.0927 | 1.1333 | 1.1594 | 1.3868 | 5 | S100 calcium binding protein A13 |
| 670 | 1.0865 | 1.4790 | 2.1189 | 1.5922 | 2.3346 | 5 | glycoprotein 49 A |
| 671 | 1.1369 | 1.4819 | 2.3374 | 1.8852 | 2.4631 | 5 | TYRO protein tyrosine kinase binding protein |
| 672 | 1.1111 | 1.1784 | 1.4885 | 1.2934 | 1.4067 | 5 | arachidonate 5-lipoxygenase activating protein |
| 673 | 1.0404 | 1.0488 | 1.4157 | 1.2505 | 1.3060 | 5 | cleavage and polyadenylation specific factor 5, 25 kD subunit |
| 674 | 1.1808 | 1.2468 | 2.1460 | 2.5342 | 2.9641 | 5 | complement component 1, q subcomponent, alpha polypeptide |
| 675 | 0.9743 | 0.9563 | 1.3347 | 1.4941 | 1.6175 | 5 | RIKEN cDNA 1200013A08 gene |
| 676 | 0.9849 | 0.9986 | 1.7538 | 2.3189 | 2.2978 | 5 | beta-2 microglobulin |
| 677 | 1.1171 | 1.0779 | 1.6506 | 1.8001 | 1.8664 | 5 | guanylate nucleotide binding protein 2 |
| 678 | 1.0166 | 0.9752 | 1.2561 | 1.3436 | 1.3418 | 5 | expressed sequence AW047581 |
| 679 | 1.0224 | 0.9359 | 1.2709 | 1.4667 | 1.3412 | 5 | metallocarboxypeptidase 1 |
| 680 | 1.0739 | 0.9786 | 1.2602 | 1.3691 | 1.3384 | 5 | expressed sequence AI448003 |
| 681 | 1.1453 | 1.1106 | 1.3561 | 1.4374 | 1.3482 | 5 | caspase 3, apoptosis related cysteine protease |
| 682 | 1.0831 | 1.1017 | 1.3415 | 1.4836 | 1.3930 | 5 | ribosomal protein S29 |
| 683 | 1.0102 | 1.0105 | 1.2104 | 1.2995 | 1.2213 | 5 | Yamaguchi sarcoma viral (v-yes) oncogene homolog |
| 684 | 0.9604 | 1.1147 | 1.1871 | 1.2848 | 1.4119 | 5 | RIKEN cDNA 1200009B18 gene |
| 685 | 0.8362 | 1.1384 | 1.4695 | 1.7288 | 2.2433 | 5 | B-cell leukemia/lymphoma 2 related protein A1b |
| 686 | 1.1090 | 1.2709 | 1.3923 | 1.4400 | 1.5966 | 5 | RIKEN cDNA 1190006C12 gene |
| 687 | 1.0209 | 1.1713 | 1.4081 | 1.4364 | 1.6875 | 5 | expressed sequence AI607846 |
| 688 | 1.1939 | 1.2368 | 1.3188 | 1.3272 | 1.4055 | 5 | proteasome (prosome, macropain) subunit, beta type 1 |
| 689 | 0.9783 | 1.0780 | 1.6032 | 1.5458 | 2.3097 | 5 | chemokine (C-C) receptor 2 |
| 690 | 1.0895 | 1.2245 | 1.9302 | 2.0222 | 2.9847 | 5 | CD52 antigen |
| 691 | 1.0296 | 1.1299 | 1.3880 | 1.4977 | 1.4916 | 5 | Unknown |
| 692 | 1.0393 | 1.1804 | 1.6343 | 1.7403 | 1.6888 | 5 | proteasome (prosome, macropain) 28 subunit, alpha |
| 693 | 0.9593 | 1.0544 | 1.2712 | 1.3496 | 1.3103 | 5 | RIKEN cDNA 2410174K12 gene |
| 694 | 0.9861 | 1.1918 | 1.5151 | 1.8749 | 1.7592 | 5 | calponin 2 |
| 695 | 1.0252 | 1.2281 | 1.4217 | 1.6469 | 1.6374 | 5 | aldehyde dehydrogenase family 1, subfamily A2 |
| 696 | 1.1009 | 1.2982 | 2.1060 | 2.0360 | 2.3479 | 5 | Fc receptor, IgE, high affinity I, gamma polypeptide |
| 697 | 1.0192 | 1.1598 | 1.3064 | 1.3476 | 1.4013 | 5 | expressed sequence AI504062 |
| 698 | 0.9578 | 2.0401 | 3.9311 | 5.1872 | 6.5144 | 5 | lysozyme |
| 699 | 0.9370 | 1.3643 | 1.8445 | 1.9401 | 2.2436 | 5 | natural killer tumor recognition sequence |
| 700 | 1.1083 | 1.2251 | 1.3376 | 1.3540 | 1.3925 | 5 | B-box and SPRY domain containing |
| 701 | 0.9443 | 1.2390 | 1.6405 | 1.6112 | 1.7935 | 5 | Fc receptor, IgG, low affinity III |
| 702 | 0.9918 | 1.1699 | 1.4482 | 1.4687 | 1.6015 | 5 | RIKEN cDNA 2700038K18 gene |
| 703 | 1.0606 | 1.2121 | 1.2325 | 1.4254 | 1.1920 | 6 | RIKEN cDNA 1700019E19 gene |
| 704 | 1.1066 | 1.1989 | 1.2372 | 1.3779 | 1.2176 | 6 | surfeit gene 4 |
| 705 | 0.9315 | 1.1788 | 1.2447 | 1.6739 | 1.1873 | 6 | RIKEN cDNA 2310075M15 gene |
| 706 | 1.2027 | 1.4701 | 1.5852 | 1.8502 | 1.4909 | 6 | guanine nucleotide binding protein, alpha inhibiting 2 |
| 707 | 0.9344 | 1.1225 | 1.1490 | 1.3265 | 1.0855 | 6 | caspase 8 |
| 708 | 1.0959 | 1.2048 | 1.3568 | 1.5922 | 1.2869 | 6 | capping protein beta 1 |
| 709 | 1.0380 | 1.1563 | 1.3441 | 1.6285 | 1.2038 | 6 | coronin, actin binding protein 1B |
| 710 | 1.0421 | 1.2388 | 1.3668 | 2.3298 | 1.2848 | 6 | amelogenin |
| 711 | 1.0830 | 1.1883 | 1.2931 | 1.5618 | 1.1971 | 6 | endoplasmic reticulum protein 29 |
| 712 | 1.0856 | 1.1567 | 1.1889 | 1.3176 | 1.1567 | 6 | downstream of tyrosine kinase 1 |
| 713 | 1.0122 | 1.2117 | 1.1438 | 1.5175 | 1.1604 | 6 | RAB11a, member RAS oncogene family |
| 714 | 1.0112 | 1.1928 | 1.2095 | 1.5860 | 1.0730 | 6 | opioid growth factor receptor |
| 715 | 1.1492 | 1.1032 | 1.3034 | 1.4873 | 1.2344 | 6 | beta-glucuronidase structural |
| 716 | 1.1432 | 1.1704 | 1.3000 | 1.4547 | 1.2248 | 6 | ESTs |
| 717 | 1.0719 | 1.0800 | 1.2977 | 1.4416 | 1.1565 | 6 | expressed sequence AW541137 |
| 718 | 1.0633 | 1.0952 | 1.3470 | 1.3650 | 1.2595 | 6 | guanine nucleotide binding protein (G protein), gamma 2 subunit |
| 719 | 1.0323 | 1.1273 | 1.4283 | 1.4902 | 1.3463 | 6 | plasminogen activator, tissue |
| 720 | 1.0174 | 1.0712 | 1.2406 | 1.3142 | 1.1995 | 6 | expressed sequence AU019833 |
| 721 | 1.0999 | 1.1124 | 1.4720 | 1.5171 | 1.2700 | 6 | melanoma antigen, family D, 2 |
| 722 | 1.0978 | 1.1379 | 1.4399 | 1.5275 | 1.2118 | 6 | dihydropyrimidinase-like 3 |
| 723 | 1.1797 | 1.2266 | 1.3528 | 1.4180 | 1.2454 | 6 | selectin, platelet (p-selectin) ligand |
| 724 | 0.9184 | 1.0715 | 1.4088 | 1.4801 | 1.1810 | 6 | granulin |
| 725 | 0.9381 | 1.0954 | 1.2682 | 1.3941 | 1.1584 | 6 | a disintegrin-like and metalloprotease (reprolysin type) with thrombospondin |
| | | | | | | | type 1 motif, 2 |
| 726 | 1.0833 | 1.2005 | 1.4448 | 1.6054 | 1.3142 | 6 | myosin light chain, alkali, nonmuscle |
| 727 | 1.0452 | 1.2868 | 1.7335 | 1.8206 | 1.2690 | 6 | complement component factor i |
| 728 | 1.1323 | 1.3474 | 1.5375 | 1.6888 | 1.2981 | 6 | small nuclear ribonucleoprotein D2 |
| 729 | 0.7812 | 1.1898 | 0.9419 | 1.2555 | 1.3221 | 6 | lysosomal-associated protein transmembrane 4A |
| 730 | 0.8744 | 1.1469 | 0.9262 | 1.2769 | 1.2876 | 6 | split hand/foot deleted gene 1 |
| 731 | 0.9975 | 1.3717 | 1.1286 | 1.7019 | 1.7636 | 6 | thrombospondin 1 |
| 732 | 1.0677 | 1.3859 | 1.6223 | 1.8310 | 1.7039 | 6 | actin, gamma 2, smooth muscle, enteric |
| 733 | 1.0888 | 1.4078 | 1.7599 | 2.0624 | 1.8261 | 6 | Unknown |
| 734 | 0.9344 | 1.4578 | 2.1769 | 3.5183 | 2.2035 | 6 | procollagen, type 1, alpha 2 |
| 735 | 0.7933 | 1.1273 | 1.6004 | 2.1567 | 1.6828 | 6 | biglycan |
| 736 | 0.9374 | 1.1525 | 1.4079 | 1.7428 | 1.4970 | 6 | Mus musculus, Similar to ribosomal protein S20, clone MGC: 6876 IMAGE: |
| | | | | | | | 2651405, mRNA, complete cds |
| 737 | 0.9686 | 1.2041 | 1.2662 | 1.5067 | 1.2539 | 6 | splicing factor 3b, subunit 1, 155 kDa |
| 738 | 0.9678 | 1.3252 | 1.3643 | 1.7055 | 1.3774 | 6 | hypothetical protein, MNCb-5210 |
| 739 | 1.0742 | 1.2512 | 1.2828 | 1.4484 | 1.2954 | 6 | proteasome (prosome, macropain) subunit, alpha type 7 |
| 740 | 1.1303 | 1.3852 | 1.4497 | 1.6362 | 1.4616 | 6 | high mobility group box 3 |
| 741 | 0.9848 | 1.3195 | 1.5136 | 1.8157 | 1.5076 | 6 | nucleophosmin 1 |
| 742 | 1.0394 | 1.2427 | 1.4044 | 1.4843 | 1.3419 | 6 | signal sequence receptor, delta |
| 743 | 0.9672 | 1.3678 | 1.7620 | 1.9661 | 1.6435 | 6 | T-box 6 |
| 744 | 0.9743 | 1.2304 | 1.3690 | 1.5300 | 1.2976 | 6 | RIKEN cDNA 4930533K18 gene |
| 745 | 1.0390 | 1.2692 | 1.4427 | 1.5827 | 1.3237 | 6 | cadherin 3 |
| 746 | 1.0108 | 1.4643 | 1.3598 | 1.6636 | 1.4907 | 6 | small inducible cytokine subfamily D, 1 |
| 747 | 0.8722 | 1.9700 | 1.6928 | 2.3710 | 2.0115 | 6 | tubulin alpha 1 |
| 748 | 0.8427 | 1.6802 | 1.3611 | 1.9522 | 1.8185 | 6 | CD24a antigen |
| 749 | 0.8687 | 1.5325 | 1.3358 | 1.6159 | 1.7194 | 6 | growth arrest and DNA-damage-inducible 45 alpha |
| 750 | 1.0626 | 1.7575 | 1.5637 | 1.8897 | 1.8508 | 6 | Unknown |
| 751 | 1.0145 | 1.8748 | 1.6219 | 2.0854 | 2.1057 | 6 | immediate early response, erythropoietin 1 |
| 752 | 0.7616 | 1.4564 | 1.1414 | 1.4722 | 1.4221 | 6 | annexin A4 |
| 753 | 1.0080 | 1.4162 | 1.2490 | 1.4871 | 1.3785 | 6 | histone deacetylase 1 |
| 754 | 0.9379 | 1.4042 | 1.4739 | 2.0339 | 1.9503 | 6 | histocompatibility 2, L region |
| 755 | 0.9656 | 1.1400 | 1.1069 | 1.2206 | 1.2019 | 6 | RAB3D, member RAS oncogene family |
| 756 | 0.8950 | 1.8132 | 1.4750 | 2.9820 | 2.5468 | 6 | elongation of very long chain fatty acids (FEN1/Elo2, SUR4/Elo3, yeast)-like 1 |
| 757 | 1.0693 | 1.2638 | 1.2420 | 1.4219 | 1.3954 | 6 | eukaryotic translation initiation factor 1A |
| 758 | 1.1316 | 1.2498 | 1.2371 | 1.3497 | 1.3131 | 6 | avian reticuloendotheliosis viral (v-rel) oncogene related B |
| 759 | 1.0173 | 1.4567 | 1.5343 | 1.7486 | 1.5702 | 6 | TG interacting factor |
| 760 | 0.9655 | 1.7159 | 1.6581 | 2.3284 | 1.8443 | 6 | ribosomal protein L12 |
| 761 | 0.8361 | 1.1938 | 1.2435 | 1.5812 | 1.4019 | 6 | interferon gamma receptor |
| 762 | 0.9506 | 1.3459 | 1.3198 | 1.6113 | 1.5104 | 6 | keratin complex 1, acidic, gene 19 |
| 763 | 0.9192 | 1.7645 | 1.6582 | 2.5436 | 2.0641 | 6 | procollagen, type XVIII, alpha 1 |
| 764 | 0.7093 | 2.1074 | 2.0978 | 4.0372 | 2.9033 | 6 | complement component 3 |
| 765 | 1.0125 | 1.3344 | 1.1188 | 1.6011 | 1.3965 | 6 | expressed sequence AW111961 |
| 766 | 1.0552 | 1.3809 | 1.2340 | 1.5831 | 1.4188 | 6 | baculoviral IAP repeat-containing 2 |
| 767 | 1.0179 | 1.2935 | 1.1881 | 1.5760 | 1.3816 | 6 | epidermal growth factor-containing fibulin-like extracellular matrix protein 1 |
| 768 | 0.9495 | 1.3974 | 1.1702 | 1.8586 | 1.4841 | 6 | ribosomal protein L18 |
| 769 | 1.0803 | 1.2121 | 1.1759 | 1.3759 | 1.2320 | 6 | RIKEN cDNA 2810430J06 gene |
| 770 | 1.0344 | 1.2179 | 1.1687 | 1.3464 | 1.2275 | 6 | golgi reassembly stacking protein 2 |
| 771 | 1.0616 | 1.6194 | 1.4664 | 2.0049 | 1.5683 | 6 | actin, alpha 1, skeletal muscle |
| 772 | 1.0373 | 1.1553 | 1.1530 | 1.3571 | 1.2929 | 6 | kinectin 1 |
| 773 | 0.8842 | 1.1615 | 1.1325 | 1.7664 | 1.5932 | 6 | histocompatibility 2, Q region locus 7 |
| 774 | 0.8593 | 1.6164 | 1.6516 | 3.2577 | 2.6599 | 6 | crystallin, mu |
| 775 | 0.9906 | 1.1392 | 1.1858 | 1.4323 | 1.3385 | 6 | leucocyte specific transcript 1 |
| 776 | 1.1755 | 1.2462 | 1.2241 | 1.3630 | 1.3044 | 6 | TATA box binding protein-like protein |
| 777 | 0.8480 | 1.3082 | 1.1638 | 2.0306 | 1.6109 | 6 | MARCKS-like protein |
| 778 | 0.8768 | 1.1194 | 1.0153 | 1.4636 | 1.2684 | 6 | metastasis associated 1-like 1 |
| 779 | 0.9204 | 1.3418 | 1.2796 | 1.8044 | 1.5230 | 6 | connective tissue growth factor |
| 780 | 1.0785 | 1.3460 | 1.3040 | 1.6682 | 1.4845 | 6 | ESTs |
| 781 | 0.9374 | 1.3245 | 1.3484 | 1.9955 | 1.5619 | 6 | vasodilator-stimulated phosphoprotein |
| 782 | 0.9010 | 1.2372 | 1.2595 | 1.8395 | 1.4802 | 6 | peptidylprolyl isomerase C-associated protein |
| 783 | 1.0568 | 1.6511 | 1.6283 | 2.7915 | 2.1974 | 6 | transgelin |
| 784 | 0.9528 | 1.2840 | 1.2833 | 1.9098 | 1.5543 | 6 | ribosomal protein S14 |
| 785 | 0.9767 | 1.1693 | 1.2733 | 1.8863 | 1.4423 | 6 | RIKEN cDNA 5133400A03 gene |
| 786 | 0.9852 | 1.1150 | 1.2153 | 1.4599 | 1.2672 | 6 | RIKEN cDNA 2610306D21 gene |
| 787 | 1.1867 | 1.2431 | 1.2465 | 1.4343 | 1.3120 | 6 | liver-specific bHLH-Zip transcription factor |
| 788 | 0.9294 | 1.0982 | 1.1319 | 1.7482 | 1.4342 | 6 | carboxypeptidase E |
| 789 | 1.0208 | 0.8748 | 0.8032 | 1.1788 | 0.9069 | 7 | deltex 1 homolog (Drosophila) |
| 790 | 0.9132 | 0.7841 | 0.6910 | 0.9989 | 0.8373 | 7 | cryptochrome 2 (photolyase-like) |
| 791 | 0.9836 | 0.9162 | 0.7819 | 1.0514 | 0.9635 | 7 | adenylate cyclase 4 |
| 792 | 1.2234 | 1.2195 | 1.0781 | 1.3454 | 1.2406 | 7 | DnaJ (Hsp40) homolog, subfamily C, member 5 |
| 793 | 0.8900 | 0.8102 | 0.8301 | 1.1757 | 1.0238 | 7 | polycystic kidney disease 1 homolog |
| 794 | 0.9399 | 0.8551 | 0.8567 | 1.2236 | 0.9813 | 7 | expressed sequence AW488255 |
| 795 | 1.1056 | 1.1485 | 1.1005 | 1.3485 | 1.1866 | 7 | Ngfi-A binding protein 2 |
| 796 | 1.0624 | 1.1238 | 0.9789 | 1.4387 | 1.1949 | 7 | Mus musculus, clone MGC: 36554 IMAGE: 4954874, mRNA, complete cds |
| 797 | 1.0273 | 1.0711 | 0.9476 | 1.4124 | 1.1986 | 7 | transformed mouse 3T3 cell double minute 2 |
| 798 | 1.0994 | 1.3428 | 1.0856 | 1.9992 | 1.5318 | 7 | small inducible cytokine A5 |
| 799 | 1.0059 | 1.1058 | 0.9659 | 1.3759 | 1.2274 | 7 | Mus musculus, clone IMAGE: 3491421, mRNA, partial cds |
| 800 | 1.0184 | 1.0863 | 0.9967 | 1.3505 | 1.2389 | 7 | Unknown |
| 801 | 1.0865 | 1.1279 | 1.0651 | 1.2945 | 1.2139 | 7 | expressed sequence AI987692 |
| 802 | 0.9384 | 0.8887 | 0.7456 | 1.2847 | 1.0899 | 7 | ALL1-fused gene from chromosome 1q |
| 803 | 0.9298 | 0.8771 | 0.7621 | 1.1161 | 0.9872 | 7 | protein tyrosine phosphatase, receptor type, B |
| 804 | 1.0172 | 0.9534 | 0.8731 | 1.4073 | 1.3397 | 7 | RIKEN cDNA 2700055K07 gene |
| 805 | 1.0252 | 1.0214 | 0.9262 | 1.3005 | 1.1695 | 7 | RIKEN cDNA 1110005N04 gene |
| 806 | 1.1757 | 1.1622 | 1.1274 | 1.3961 | 1.3171 | 7 | hypothetical protein, MGC: 6957 |
| 807 | 1.1705 | 1.5789 | 2.1648 | 1.4597 | 1.0748 | 8 | ribosomal protein L41 |
| 808 | 1.0635 | 1.3540 | 1.8472 | 1.0696 | 0.9349 | 8 | karyopherin (importin) alpha 2 |
| 809 | 1.0256 | 1.3089 | 1.7153 | 1.0984 | 0.9137 | 8 | 3-phosphoglycerate dehydrogenase |
| 810 | 1.0346 | 1.3321 | 1.6196 | 1.1644 | 1.0462 | 8 | nuclease sensitive element binding protein 1 |
| 811 | 0.9787 | 1.1078 | 1.2493 | 1.0180 | 0.9729 | 8 | Unknown |
| 812 | 1.0001 | 1.2154 | 1.3699 | 1.1075 | 1.0554 | 8 | fragile histidine triad gene |
| 813 | 1.0656 | 1.2748 | 1.5250 | 1.2011 | 1.1393 | 8 | RIKEN cDNA 1200014I03 gene |
| 814 | 0.9228 | 1.1853 | 1.5148 | 1.0335 | 0.9811 | 8 | forkhead box M1 |
| 815 | 0.9805 | 3.4757 | 6.3976 | 2.3798 | 1.3904 | 8 | secreted phosphoprotein 1 |
| 816 | 1.1463 | 1.5485 | 1.8329 | 1.4366 | 1.2921 | 8 | Unknown |
| 817 | 1.0634 | 1.4566 | 1.6696 | 1.3192 | 1.0792 | 8 | ribosomal protein L36 |
| 818 | 0.9823 | 1.2685 | 1.4028 | 1.1183 | 1.0011 | 8 | retinoblastoma binding protein 7 |
| 819 | 0.9367 | 1.4419 | 1.5893 | 1.1107 | 1.0894 | 8 | FK506 binding protein 10 (65 kDa) |
| 820 | 0.7917 | 1.6376 | 1.8312 | 1.0070 | 0.9740 | 8 | heme oxygenase (decycling) 1 |
| 821 | 1.0398 | 2.4542 | 2.5246 | 1.3065 | 1.2043 | 8 | high mobility group AT-hook 1 |
| 822 | 1.0502 | 1.2580 | 1.2989 | 1.0864 | 1.0692 | 8 | inhibin beta-B |
| 823 | 1.0485 | 1.3901 | 1.4398 | 1.1152 | 1.1263 | 8 | myeloid-associated differentiation marker |
| 824 | 0.9600 | 1.1952 | 1.2455 | 0.9994 | 1.0090 | 8 | RIKEN cDNA 1300019I21 gene |
| 825 | 1.0409 | 1.4146 | 1.5614 | 1.1026 | 1.1820 | 8 | protein phosphatase 1, catalytic subunit, alpha isoform |
| 826 | 1.0368 | 1.4925 | 1.8381 | 1.1524 | 1.2176 | 8 | Unknown |
| 827 | 1.0262 | 1.5053 | 1.6804 | 1.2337 | 1.2622 | 8 | numb gene homolog (Drosophila) |
| 828 | 0.9552 | 1.2544 | 1.3881 | 1.0502 | 1.1517 | 8 | enhancer of zeste homolog 2 (Drosophila) |
| 829 | 1.1289 | 1.2774 | 1.4450 | 1.0867 | 1.1240 | 8 | CCCTC-binding factor |
| 830 | 0.9267 | 1.2192 | 1.6018 | 0.9633 | 0.9769 | 8 | RIKEN cDNA 2600017H24 gene |
| 831 | 1.1364 | 1.3499 | 1.4842 | 1.1054 | 1.0905 | 8 | ESTs |
| 832 | 1.1178 | 1.3461 | 1.5230 | 1.1353 | 1.0800 | 8 | RIKEN cDNA 1110054A24 gene |
| 833 | 1.0265 | 1.2562 | 1.3312 | 1.0744 | 0.9661 | 8 | mutS homolog 6 (E. coli) |
| 834 | 0.9568 | 1.1392 | 1.1933 | 0.9676 | 0.8936 | 8 | TRAF-interacting protein |
| 835 | 0.9733 | 1.1567 | 1.2601 | 0.9746 | 0.9198 | 8 | cyclin E1 |
| 836 | 0.9535 | 1.2877 | 1.3981 | 0.9579 | 0.8719 | 8 | RIKEN cDNA 1810058K22 gene |
| 837 | 1.0752 | 1.5091 | 1.6571 | 1.0736 | 1.0018 | 8 | erythroid differentiation regulator |
| 838 | 0.9263 | 1.2611 | 1.2404 | 0.9111 | 0.9423 | 8 | leukotriene C4 synthase |
| 839 | 1.0243 | 1.2567 | 1.2798 | 0.9961 | 0.9792 | 8 | RIKEN cDNA 4921537D05 gene |
| 840 | 1.0986 | 1.2793 | 1.3604 | 1.0644 | 1.0840 | 8 | DNA segment, Chr 17, human D6S56E 2 |
| 841 | 1.1115 | 1.2630 | 1.3067 | 1.1052 | 1.1143 | 8 | N-acetylglucosamine kinase |
| 842 | 1.0186 | 1.1338 | 1.1682 | 1.0164 | 1.0152 | 8 | syntrophin, basic 2 |
| 843 | 1.0902 | 1.3673 | 1.2716 | 1.1692 | 1.1034 | 8 | ESTs |
| 844 | 0.9755 | 1.4063 | 1.2003 | 1.1230 | 0.9815 | 8 | RIKEN cDNA 3230402E02 gene |
| 845 | 1.0026 | 1.4399 | 1.2713 | 1.1845 | 0.9994 | 8 | karyopherin (importin) beta 3 |
| 846 | 0.7846 | 0.8672 | 0.8370 | 0.8170 | 0.7820 | 8 | ESTs, Weakly similar to MAJOR URINARY PROTEIN 4 |
| | | | | | | | PRECURSOR (M. musculus) |
| 847 | 1.0338 | 2.0784 | 1.7794 | 1.4405 | 1.0162 | 8 | RIKEN cDNA 2610301D06 gene |
| 848 | 1.1081 | 1.5247 | 1.4167 | 1.2958 | 1.0599 | 8 | mini chromosome maintenance deficient 2 (S. cerevisiae) |
| 849 | 0.9863 | 1.4189 | 1.3009 | 1.1512 | 1.0359 | 8 | SWI/SNF related, matrix associated, actin dependent regulator of chromatin, |
| | | | | | | | subfamily a, member 5 |
| 850 | 0.8998 | 1.6631 | 1.5009 | 1.1670 | 0.9255 | 8 | mini chromosome maintenance deficient 5 (S. cerevisiae) |
| 851 | 0.9833 | 1.3582 | 1.2973 | 1.1468 | 0.9982 | 8 | ESTs, Weakly similar to TYROSINE-PROTEIN KINASE JAK3 (M. musculus) |
| 852 | 0.9157 | 1.3667 | 1.3004 | 1.1916 | 0.9532 | 8 | Unknown |
| 853 | 0.9737 | 1.4200 | 1.3047 | 1.2093 | 1.0306 | 8 | smoothelin |
| 854 | 0.9585 | 1.3997 | 1.2746 | 1.2102 | 1.0367 | 8 | ribosomal protein S6 kinase, 90 kD, polypeptide 4 |
| 855 | 1.0123 | 1.6805 | 1.5073 | 1.4289 | 1.2023 | 8 | RIKEN cDNA 2510015F0I gene |
| 856 | 0.9089 | 1.8163 | 1.6095 | 1.4457 | 1.1461 | 8 | syndecan 1 |
| 857 | 0.9122 | 1.2824 | 1.2224 | 1.0872 | 1.0290 | 8 | regulator for ribosome resistance homolog (S. cerevisiae) |
| 858 | 0.9298 | 1.2509 | 1.1873 | 1.0990 | 1.0063 | 8 | damage specific DNA binding protein 1 (127 kDa) |
| 859 | 1.0299 | 1.3535 | 1.2718 | 1.1233 | 1.0826 | 8 | myosin Ic |
| 860 | 1.0571 | 1.7370 | 1.6344 | 1.2004 | 1.1026 | 8 | FK506 binding protein 1a (12 kDa) |
| 861 | 0.9988 | 1.5675 | 1.4768 | 1.1448 | 1.0528 | 8 | apurinic/apyrimidinic endonuclease |
| 862 | 1.0526 | 1.8638 | 1.5916 | 1.1274 | 1.0620 | 8 | RIKEN cDNA 4930542G03 gene |
| 863 | 0.8926 | 1.4296 | 1.2322 | 0.9500 | 0.8629 | 8 | expressed sequence AA409944 |
| 864 | 1.0256 | 1.3651 | 1.3109 | 1.0412 | 0.9988 | 8 | RIKEN cDNA 0610041E09 gene |
| 865 | 1.0822 | 1.9930 | 1.6940 | 1.1588 | 0.9855 | 8 | cyclin-dependent kinase inhibitor 1A (P21) |
| 866 | 0.9237 | 1.5163 | 1.3416 | 0.9375 | 0.8375 | 8 | DNA methyltransferase (cytosine-5) 1 |
| 867 | 1.1364 | 1.7778 | 1.9225 | 1.3915 | 1.0715 | 8 | expressed sequence AL022757 |
| 868 | 0.9705 | 1.3248 | 1.3714 | 1.0729 | 0.9268 | 8 | pyruvate kinase 3 |
| 869 | 0.9647 | 1.1680 | 1.1923 | 1.0358 | 0.9426 | 8 | serine protease inhibitor, Kunitz type 1 |
| 870 | 0.9876 | 1.1944 | 1.2388 | 1.1063 | 0.9943 | 8 | UDP-Gal:betaGlcNAc beta 1,4-galactosyltransferase, polypeptide 2 |
| 871 | 0.9515 | 1.1453 | 1.1541 | 1.0396 | 0.9548 | 8 | mutS homolog 2 (E. coli) |
| 872 | 1.1114 | 2.2402 | 2.1113 | 1.2738 | 0.8502 | 8 | serum amyloid A 3 |
| 873 | 1.0317 | 1.3792 | 1.3435 | 1.0777 | 0.9710 | 8 | eukaryotic translation initiation factor 3, subunit 4 (delta, 44 kDa) |
| 874 | 0.8893 | 1.3380 | 1.3031 | 0.9826 | 0.8612 | 8 | retinoblastoma-like 1 (p107) |
| 875 | 1.1208 | 1.8190 | 1.8661 | 1.2287 | 0.9901 | 8 | mini chromosome maintenance deficient (S. cerevisiae) |
| 876 | 1.1830 | 1.5507 | 1.5841 | 1.2237 | 1.1306 | 8 | ribosomal protein S26 |
| 877 | 0.8906 | 1.4498 | 1.2272 | 1.0730 | 1.1077 | 8 | RIKEN cDNA 0610016J10 gene |
| 878 | 0.9239 | 1.7468 | 1.4637 | 1.1897 | 1.2078 | 8 | phospholipid scramblase 1 |
| 879 | 1.0531 | 3.7822 | 2.8146 | 1.7527 | 1.7093 | 8 | S100 calcium binding protein A10 (calpactin) |
| 880 | 0.9242 | 1.4141 | 1.2747 | 1.1096 | 1.0919 | 8 | RIKEN cDNA 2810047L02 gene |
| 881 | 0.9461 | 1.7827 | 1.2865 | 1.2276 | 1.1364 | 8 | group specific component |
| 882 | 0.8998 | 1.5321 | 1.2290 | 1.1541 | 1.0309 | 8 | Mus musculus, Similar to hypothetical protein FLJ20335, clone MGC: 28912 |
| | | | | | | | image: 4922274, mRNA, complete cds |
| 883 | 0.9879 | 2.0588 | 1.5204 | 1.3136 | 1.2219 | 8 | colony stimulating factor 1 (macrophage) |
| 884 | 1.0047 | 2.2985 | 1.7301 | 1.4129 | 1.2822 | 8 | cold shock domain protein A |
| 885 | 0.9698 | 2.1108 | 1.6130 | 1.2894 | 1.1897 | 8 | flotillin 1 |
| 886 | 0.9661 | 1.7268 | 1.4417 | 1.2155 | 1.1198 | 8 | eukaryotic translation initiation factor 5A |
| 887 | 0.9258 | 1.5600 | 1.3168 | 1.0144 | 1.1043 | 8 | NIMA (never in mitosis gene a)-related expressed kinase 6 |
| 888 | 0.9176 | 1.6345 | 1.3237 | 1.0002 | 1.0799 | 8 | G1 to phase transition 1 |
| 889 | 0.9109 | 1.9203 | 1.3751 | 1.1123 | 1.1116 | 8 | chaperonin subunit 3 (gamma) |
| 890 | 0.8483 | 2.3992 | 1.6048 | 1.0559 | 1.0729 | 8 | RIKEN cDNA 2610305D13 gene |
| 891 | 0.9730 | 1.3794 | 1.2046 | 1.0602 | 1.0799 | 8 | thioredoxin-like (32 kD) |
| 892 | 1.0604 | 1.7694 | 1.6202 | 1.1721 | 1.2007 | 8 | breakpoint cluster region protein 1 |
| 893 | 1.0014 | 1.2278 | 1.2144 | 1.0377 | 1.0589 | 8 | SMC (structural maintenance of chromosomes 1)-like 1 (S. cerevisiae) |
| 894 | 0.7965 | 1.2243 | 1.1858 | 0.8377 | 0.8802 | 8 | Kruppel-like factor 5 |
| 895 | 1.0803 | 1.3750 | 1.3074 | 1.1710 | 1.1562 | 8 | RIKEN CDNA 2510001A17 gene |
| 896 | 1.0082 | 1.3212 | 1.2504 | 1.0867 | 1.0966 | 8 | protease (prosome, macropain) 26S subunit, ATPase 1 |
| 897 | 0.9992 | 1.1627 | 1.1318 | 1.0470 | 1.0467 | 8 | RIKEN cDNA 1110003H02 gene |
| 898 | 0.9447 | 1.2588 | 1.2104 | 1.0081 | 1.0547 | 8 | RIKEN cDNA 5430416A05 gene |
| 899 | 1.0011 | 2.0612 | 1.8059 | 1.2030 | 1.3241 | 8 | expressed sequence R75232 |
| 900 | 0.9157 | 1.4018 | 1.2908 | 1.0143 | 1.0631 | 8 | platelet derived growth factor receptor, beta polypeptide |
| 901 | 0.8712 | 1.5231 | 1.3539 | 0.9955 | 1.1175 | 8 | exportin 1, CRM1 homolog (yeast) |
| 902 | 0.9824 | 1.3532 | 1.2566 | 1.0814 | 1.1199 | 8 | adenylosuccinate synthetase 2, non muscle |
| 903 | 1.0426 | 2.5548 | 1.2975 | 0.9628 | 0.8206 | 8 | crystallin, alpha B |
| 904 | 1.0750 | 1.2433 | 1.1610 | 1.0587 | 1.0001 | 8 | RIKEN cDNA 2610029K21 gene |
| 905 | 0.8633 | 1.4897 | 1.1450 | 0.9054 | 0.7761 | 8 | peroxiredoxin 5 |
| 906 | 0.9973 | 1.7128 | 1.3332 | 1.0895 | 0.8870 | 8 | glutathione S-transferase, mu 6 |
| 907 | 0.9213 | 1.3955 | 1.2021 | 0.9890 | 0.9673 | 8 | ESTs |
| 908 | 0.9483 | 1.7476 | 1.3518 | 1.0398 | 0.9682 | 8 | Mus musculus, clone IMAGE: 4486265, mRNA, partial cds |
| 909 | 0.9987 | 3.3629 | 1.8313 | 1.1715 | 1.0354 | 8 | metallothionein 2 |
| 910 | 0.9659 | 1.3942 | 1.1693 | 1.0673 | 0.9683 | 8 | ESTs, Moderately similar to T00381 KIAA0633 protein (H. sapiens) |
| 911 | 0.9254 | 1.7080 | 1.2838 | 1.1494 | 1.0299 | 8 | RIKEN cDNA 2610524K04 gene |
| 912 | 0.9236 | 1.7544 | 1.2779 | 1.1024 | 0.9863 | 8 | tuftelin 1 |
| 913 | 1.6779 | 3.3827 | 2.0004 | 1.9197 | 1.7790 | 8 | cysteine rich protein 61 |
| 914 | 0.9191 | 1.8726 | 1.2485 | 0.8887 | 0.9707 | 8 | spermidine synthase |
| 915 | 1.0491 | 1.7138 | 1.2456 | 1.0594 | 1.0698 | 8 | fibrillarin |
| 916 | 1.0589 | 1.3100 | 1.1440 | 1.0864 | 1.0835 | 8 | polypyrimidine tract binding protein 1 |
| 917 | 1.0043 | 1.3546 | 1.3814 | 1.0214 | 1.2202 | 8 | proteoglycan, secretory granule |
| 918 | 0.9100 | 1.3713 | 1.2753 | 0.9012 | 1.1238 | 8 | RIKEN cDNA 1100001F19 gene |
| 919 | 1.0474 | 1.3899 | 1.3758 | 1.0370 | 1.1680 | 8 | phosphatidylinositol transfer protein |
| 920 | 0.9266 | 1.2615 | 1.2228 | 0.9098 | 1.0594 | 8 | Ral-interacting protein 1 |
| 921 | 1.0015 | 1.1566 | 1.2123 | 0.9398 | 1.0363 | 8 | serine/threonine protein kinase CISK |
| 922 | 1.1089 | 1.2420 | 1.2912 | 1.0800 | 1.1813 | 8 | septin 8 |
| 923 | 0.9884 | 1.2165 | 1.2276 | 0.9395 | 1.0978 | 8 | splicing factor, arginine/serine-rich 2 (SC-35) |
| 924 | 0.9563 | 1.2095 | 1.2477 | 0.9184 | 1.0629 | 8 | RIKEN cDNA 1300018I05 gene |
| 925 | 1.0527 | 1.3395 | 1.1731 | 0.9617 | 1.0150 | 8 | microtubule associated testis specific serine/threonine protein kinase |
| 926 | 0.9314 | 1.3143 | 1.1483 | 0.8630 | 0.9104 | 8 | spermatogenesis associated factor |
| 927 | 0.8097 | 2.0123 | 1.3849 | 0.7516 | 0.8220 | 8 | phospholipase A2, group IB, pancreas |
| 928 | 1.0119 | 1.4082 | 1.2014 | 0.9940 | 1.0231 | 8 | proteasome (prosome, macropain) 26S subunit, non-ATPase, 13 |
| 929 | 1.0211 | 1.2430 | 1.1788 | 0.9232 | 0.9643 | 8 | RIKEN cDNA 0610007L01 gene |
| 930 | 1.0922 | 1.5011 | 1.3576 | 0.9118 | 0.9694 | 8 | tumor necrosis factor receptor superfamily, member 10b |
| 931 | 0.9632 | 2.3385 | 1.4739 | 0.6976 | 0.7120 | 8 | metallothionein 1 |
| 932 | 1.1409 | 1.4503 | 1.2784 | 1.0595 | 1.0593 | 8 | RIKEN cDNA 1810038D15 gene |
| 933 | 1.0397 | 1.5167 | 1.3167 | 0.9642 | 0.9628 | 8 | MYB binding protein (P160) 1a |
| 934 | 1.0788 | 1.4643 | 1.2926 | 0.9942 | 0.9723 | 8 | N-acetylneuraminate pyruvate lyase |
| 935 | 1.0434 | 1.4442 | 1.2448 | 0.9364 | 1.1521 | 8 | zuotin related factor 2 |
| 936 | 1.0222 | 1.3369 | 1.1789 | 0.9739 | 1.1195 | 8 | poly(rC) binding protein 1 |
| 937 | 1.0415 | 1.4282 | 1.2706 | 0.9824 | 1.0942 | 8 | heat shock 70 kDa protein 4 |
| 938 | 1.0332 | 1.4662 | 1.3099 | 0.9474 | 1.1177 | 8 | RIKEN cDNA 2810409H07 gene |
| 939 | 1.0604 | 1.4091 | 1.2679 | 1.0375 | 1.1612 | 8 | CDK2 (cyclin-dependent kinase 2)-asscoaited protein 1 |
| 940 | 1.0057 | 1.2417 | 1.1350 | 0.9895 | 1.0433 | 8 | RIKEN cDNA 2310079C17 gene |
| 941 | 1.1422 | 1.4354 | 1.3050 | 0.9770 | 1.1061 | 8 | poliovirus receptor-related 3 |
| 942 | 0.9717 | 1.2575 | 1.2031 | 0.9203 | 1.0098 | 8 | RIKEN cDNA 6720463E02 gene |
| 943 | 1.0358 | 1.3252 | 1.2681 | 0.9331 | 1.0866 | 8 | ESTs |
| 944 | 1.0070 | 1.3308 | 1.2793 | 0.8952 | 1.0254 | 8 | RIKEN cDNA 2810004N23 gene |
| 945 | 0.8562 | 0.9086 | 0.6539 | 0.7555 | 0.6474 | 9 | acyl-Coenzyme A dehydrogenase, very long chain |
| 946 | 0.9061 | 0.8925 | 0.7442 | 0.7410 | 0.6812 | 9 | signaling intermediate in Toll pathway-evolutionarily conserved |
| 947 | 0.8913 | 0.8476 | 0.6680 | 0.7499 | 0.5878 | 9 | Unknown |
| 948 | 0.6959 | 0.5637 | 0.2599 | 0.4330 | 0.4496 | 9 | cytochrome P450, 2a4 |
| 949 | 0.9439 | 0.9168 | 0.7779 | 0.8257 | 0.8673 | 9 | vascular endothelial growth factor A |
| 950 | 1.1024 | 0.8707 | 0.7376 | 0.8276 | 0.7476 | 9 | caspase 1 |
| 951 | 1.0198 | 0.8330 | 0.6820 | 0.7713 | 0.6831 | 9 | upstream transcription factor 1 |
| 952 | 0.8934 | 0.7274 | 0.5571 | 0.6259 | 0.6453 | 9 | Mus musculus, Similar to KIAA1075 protein, clone IMAGE: 5099327, |
| | | | | | | | mRNA, partial cds |
| 953 | 0.9912 | 0.9011 | 0.8155 | 0.8613 | 0.8507 | 9 | ESTs |
| 954 | 1.0566 | 0.8549 | 0.6306 | 0.7014 | 0.6514 | 9 | Unknown |
| 955 | 0.9210 | 0.7784 | 0.5715 | 0.6506 | 0.6378 | 9 | expressed sequence AW261723 |
| 956 | 1.1198 | 0.9949 | 0.6888 | 0.8164 | 0.7860 | 9 | ESTs |
| 957 | 0.9687 | 0.8107 | 0.7639 | 0.7686 | 0.6485 | 10 | RIKEN cDNA 1700015P13 gene |
| 958 | 0.9378 | 0.8541 | 0.8934 | 0.7955 | 0.7153 | 10 | polymerase, gamma |
| 959 | 1.1040 | 0.9020 | 0.7961 | 0.5188 | 0.3967 | 10 | growth arrest and DNA-damage-inducible 45 gamma |
| 960 | 0.8252 | 0.8038 | 0.7707 | 0.7536 | 0.6861 | 10 | Unknown |
| 961 | 1.0298 | 0.8636 | 0.8640 | 0.7991 | 0.8070 | 10 | single Ig IL-1 receptor related protein |
| 962 | 1.0620 | 0.6586 | 0.5847 | 0.4552 | 0.4282 | 10 | sex-lethal interactor homolog (Drosophila) |
| 963 | 0.8831 | 0.6946 | 0.6819 | 0.5761 | 0.5869 | 10 | carnitine palmitoyltransferase 1, liver |
| 964 | 0.9346 | 0.8586 | 0.8429 | 0.7803 | 0.7996 | 10 | Unknown |
| 965 | 0.8992 | 0.7099 | 0.6082 | 0.5737 | 0.5496 | 10 | UDP-glucuronosyltransferase 1 family, member 1 |
| 966 | 1.0169 | 0.8792 | 0.7612 | 0.7490 | 0.7008 | 10 | D-amino acid oxidase |
| 967 | 1.0497 | 0.8466 | 0.7016 | 0.6124 | 0.6259 | 10 | RIKEN cDNA 6530411B15 gene |
| 968 | 1.0244 | 0.9427 | 0.8750 | 0.7907 | 0.8257 | 10 | expressed sequence AI661919 |
| 969 | 0.9882 | 0.8769 | 0.9026 | 0.8647 | 0.8679 | 10 | f-box only protein 3 |
| 970 | 1.1131 | 0.8263 | 0.9425 | 0.7617 | 0.7845 | 10 | cytochrome c oxidase, subunit VIIIa |
| 971 | 0.9328 | 0.5563 | 0.6746 | 0.5572 | 0.4357 | 10 | glutamine synthetase |
| 972 | 1.2090 | 0.7128 | 0.9213 | 0.7013 | 0.5613 | 10 | FXYD domain-containing ion transport regulator 2 |
| 973 | 1.0048 | 0.7884 | 0.7950 | 0.7500 | 0.6439 | 10 | DNA segment, Chr 18, Wayne State University 181, expressed |
| 974 | 0.8833 | 0.7058 | 0.7194 | 0.6722 | 0.6111 | 10 | expressed sequence AI746547 |
| 975 | 1.0050 | 0.8164 | 0.8458 | 0.7260 | 0.6838 | 10 | solute carrier family 7 (cationic amino acid transporter, y+ system), member 7 |
| 976 | 0.7740 | 0.4108 | 0.4826 | 0.3507 | 0.3230 | 10 | glutamine synthetase |
| 977 | 0.9802 | 0.7412 | 0.7884 | 0.6852 | 0.6334 | 10 | transmembrane protein 8 (five membrane-spanning domains) |
| 978 | 1.1106 | 0.7079 | 1.0926 | 0.4646 | 0.6528 | 10 | cytochrome P450, 2d9 |
| 979 | 0.9894 | 0.7983 | 0.9261 | 0.6956 | 0.7315 | 10 | solute carrier family 25 (mitochondrial carrier; adenine nucleotide translocator), |
| | | | | | | | member 13 |
| 980 | 0.9768 | 0.8323 | 0.9159 | 0.7133 | 0.7878 | 10 | expressed sequence AI593524 |
| 981 | 1.0048 | 0.9212 | 0.9184 | 0.7053 | 0.8224 | 10 | hydroxysteroid 17-beta dehydrogenase 7 |
| 982 | 1.0054 | 0.8319 | 0.9425 | 0.6684 | 0.8122 | 10 | histone gene complex 2 |
| 983 | 0.8737 | 0.7926 | 0.8300 | 0.6773 | 0.7674 | 10 | Mus musculus, clone MGC: 18871 IMAGE: 4234793, mRNA, complete cds |
| 984 | 1.2340 | 0.9877 | 1.0330 | 0.7811 | 0.7963 | 10 | arachidonate 12-lipoxygenase, pseudogene 2 |
| 985 | 1.0932 | 0.8639 | 0.9127 | 0.5786 | 0.5608 | 10 | upregulated during skeletal muscle growth 5 |
| 986 | 1.0165 | 0.8971 | 0.9690 | 0.6225 | 0.6251 | 10 | Unknown |
| 987 | 1.0490 | 0.9308 | 0.8611 | 0.6815 | 0.6822 | 10 | gap junction membrane channel protein beta 2 |
| 988 | 0.9026 | 0.8951 | 0.6761 | 0.5261 | 0.5492 | 10 | alcohol dehydrogenase 4 (class II), pi polypeptide |
| 989 | 1.0225 | 0.9839 | 0.8840 | 0.7646 | 0.7836 | 10 | Mus musculus, Similar to hypothetical protein MGC4368, clone MGC: 28978 |
| | | | | | | | IMAGE: 4503381, mRNA, complete cds |
| 990 | 0.9773 | 0.8844 | 0.7487 | 0.6177 | 0.6086 | 10 | S-adenosylhomocysteine hydrolase |
| 991 | 0.9271 | 0.9204 | 0.6886 | 0.5611 | 0.5436 | 10 | period homolog 1 (Drosophila) |
| 992 | 0.9664 | 0.9156 | 0.7380 | 0.6360 | 0.6001 | 10 | ESTs, Moderately similar to SEC7 homolog (Homo sapiens) (H. sapiens) |
| 993 | 0.8393 | 0.8046 | 0.7230 | 0.6275 | 0.6776 | 10 | hepatic nuclear factor 4 |
| 994 | 1.0081 | 0.9686 | 0.8565 | 0.6358 | 0.7229 | 10 | macrophage migration inhibitory factor |
| 995 | 0.9571 | 0.9154 | 0.8538 | 0.6816 | 0.7615 | 10 | neural precursor cell expressed, developmentally down-regulated gene 4a |
| 996 | 0.9963 | 0.9563 | 0.8722 | 0.6864 | 0.7705 | 10 | serine hydroxymethyl transferase 1 (soluble) |
| 997 | 0.9200 | 0.8715 | 0.8570 | 0.7089 | 0.7528 | 10 | DNA segment, Chr 5, Wayne State University 31, expressed |
| 998 | 1.0673 | 1.0749 | 0.9741 | 0.3763 | 0.4696 | 10 | serum/glucocorticoid regulated kinase |
| 999 | 0.9406 | 0.9407 | 0.8980 | 0.7114 | 0.7832 | 10 | RAR-related orphan receptor alpha |
| 1000 | 1.0031 | 0.9089 | 0.7904 | 0.7543 | 0.9717 | 11 | Mus musculus, hypothetical protein MGC11287 similar to ribosomal protein S6 |
| | | | | | | | kinase,, clone MGC: 28043 IMAGE: 3672127, mRNA, complete cds |
| 1001 | 0.9025 | 0.8411 | 0.7798 | 0.7683 | 0.8986 | 11 | ESTs, Weakly similar to JC7182 Na+-dependent vitamin C (H. sapiens) |
| 1002 | 1.0356 | 0.7156 | 0.5305 | 0.5273 | 0.8063 | 11 | CEA-related cell adhesion molecule 2 |
| 1003 | 0.9586 | 0.8592 | 0.6928 | 0.7362 | 0.8763 | 11 | Mus musculus, clone IMAGE: 3586777, mRNA, partial cds |
| 1004 | 0.9311 | 0.8193 | 0.6879 | 0.7312 | 0.8855 | 11 | low density lipoprotein receptor-related protein 6 |
| 1005 | 0.8639 | 0.6973 | 0.6641 | 0.6941 | 0.8126 | 11 | Mus musculus, clone MGC: 6545 IMAGE: 2655444, mRNA, complete cds |
| 1006 | 1.0417 | 0.9110 | 0.8783 | 0.9056 | 1.0118 | 11 | ESTs |
| 1007 | 0.8410 | 0.6338 | 0.6314 | 0.6327 | 0.8084 | 11 | acyl-Coenzyme A dehydrogenase, short/branched chain |
| 1008 | 1.0358 | 0.8301 | 0.8198 | 0.8384 | 1.0072 | 11 | RIKEN cDNA 2310004I03 gene |
| 1009 | 0.9453 | 0.7680 | 0.7480 | 0.7105 | 0.8614 | 11 | ATPase, H+ transporting, lysosomal (vacuolar proton pump), alpha 70 kDa, |
| | | | | | | | isoform 1 |
| 1010 | 1.0184 | 0.6622 | 0.6123 | 0.5889 | 0.8067 | 11 | superoxide dismutase 2, mitochondrial |
| 1011 | 1.0905 | 0.8205 | 0.7908 | 0.7760 | 0.9682 | 11 | RIKEN cDNA D630002J15 gene |
| 1012 | 1.0518 | 0.6570 | 0.5914 | 0.5503 | 0.9616 | 11 | aquaporin 2 |
| 1013 | 0.8270 | 0.6440 | 0.6076 | 0.5833 | 0.7900 | 11 | CEA-related cell adhesion molecule 1 |
| 1014 | 0.9791 | 0.6898 | 0.7041 | 0.5938 | 0.9095 | 11 | expressed sequence AI844685 |
| 1015 | 0.9384 | 0.7774 | 0.7589 | 0.7022 | 0.9073 | 11 | ATPase, H+/K+ transporting, alpha polypeptide |
| 1016 | 1.1805 | 0.7019 | 0.5323 | 0.4116 | 0.7825 | 11 | calbindin-D9K |
| 1017 | 0.9968 | 0.8982 | 0.8657 | 0.7889 | 0.9085 | 11 | RIKEN cDNA 9030612K14 gene |
| 1018 | 0.9356 | 0.7407 | 0.7319 | 0.6802 | 0.8112 | 11 | ESTs |
| 1019 | 1.0822 | 0.7842 | 0.7482 | 0.6598 | 0.8558 | 11 | cytochrome c oxidase, subunit VIc |
| 1020 | 1.1006 | 0.7344 | 0.7703 | 0.6204 | 0.8251 | 11 | AU RNA binding protein/enoyl-coenzyme A hydratase |
| 1021 | 0.9895 | 0.8642 | 0.8764 | 0.8166 | 0.9034 | 11 | prohibitin |
| 1022 | 0.9992 | 0.6927 | 0.7053 | 0.6264 | 0.7778 | 11 | RIKEN cDNA 2700043D08 gene |
| 1023 | 1.1460 | 0.7980 | 0.7977 | 0.6972 | 0.8791 | 11 | dopa decarboxylase |
| 1024 | 1.0876 | 0.8549 | 0.7929 | 0.7021 | 0.8604 | 11 | ESTs, Weakly similar to ADT1 MOUSE ADP, ATP CARRIER PROTEIN, |
| | | | | | | | HEART/SKELETAL MUSCLE ISOFORM T1 (M. musculus) |
| 1025 | 1.0466 | 0.9330 | 0.8966 | 0.8504 | 0.9389 | 11 | expressed sequence AI117581 |
| 1026 | 0.9960 | 0.7530 | 0.6676 | 0.6305 | 0.7530 | 11 | ESTs, Weakly similar to TYROSINE-PROTEIN KINASE JAK3 (M. musculus) |
| 1027 | 0.9886 | 0.8343 | 0.7855 | 0.7688 | 0.8452 | 11 | PCTAIRE-motif protein kinase 3 |
| 1028 | 0.6974 | 0.4804 | 0.4424 | 0.3964 | 0.4776 | 11 | homocysteine-inducible, endoplasmic reticulum stress-inducible, ubiquitin-like |
| | | | | | | | domain member 1 |
| 1029 | 0.9916 | 0.7285 | 0.6752 | 0.6177 | 0.7502 | 11 | solute carrier family 22 (organic cation transporter), member 4 |
| 1030 | 0.9625 | 0.7216 | 0.6321 | 0.5149 | 0.6056 | 11 | RIKEN cDNA 9530089B04 gene |
| 1031 | 0.9471 | 0.7616 | 0.6508 | 0.5799 | 0.6966 | 11 | solute carrier family 26, member 4 |
| 1032 | 0.9952 | 0.7110 | 0.5728 | 0.4458 | 0.5965 | 11 | kallikrein 6 |
| 1033 | 0.9992 | 0.7903 | 0.8121 | 0.6480 | 0.7357 | 11 | expressed sequence AI504961 |
| 1034 | 0.9609 | 0.8079 | 0.8093 | 0.6809 | 0.7884 | 11 | expressed sequence AV046379 |
| 1035 | 0.9621 | 0.8559 | 0.8659 | 0.7762 | 0.8606 | 11 | ESTs |
| 1036 | 1.0417 | 0.9264 | 0.8514 | 0.6947 | 0.9882 | 11 | sideroflexin 1 |
| 1037 | 0.9864 | 0.8172 | 0.7755 | 0.6581 | 0.9205 | 11 | RIKEN cDNA 5133401H06 gene |
| 1038 | 0.8703 | 0.7712 | 0.7184 | 0.6293 | 0.8410 | 11 | RIKEN cDNA 1500041J02 gene |
| 1039 | 0.8966 | 0.8619 | 0.7604 | 0.7419 | 0.7980 | 11 | pyruvate kinase liver and red blood cell |
| 1040 | 1.0614 | 1.0054 | 0.6685 | 0.5872 | 0.7662 | 11 | glutathione S-transferase, alpha 4 |
| 1041 | 0.8833 | 0.7691 | 0.6539 | 0.6345 | 0.7495 | 11 | ESTs, Moderately similar to T08673 hypothetical protein |
| | | | | | | | DKFZp564C0222.1 (H. sapiens) |
| 1042 | 0.7851 | 0.7664 | 0.7305 | 0.7205 | 0.7619 | 11 | period homolog 1 (Drosophila) |
| 1043 | 0.9252 | 0.9021 | 0.7495 | 0.6509 | 0.8352 | 11 | heat shock protein, 105 kDa |
| 1044 | 0.9903 | 0.9088 | 0.8075 | 0.7381 | 0.8826 | 11 | kinesin family member 21A |
| 1045 | 0.9834 | 0.9108 | 0.8079 | 0.7134 | 0.8447 | 11 | expressed sequence AI844876 |
| 1046 | 1.0546 | 1.4947 | 1.3198 | 1.3810 | 1.0548 | 12 | RIKEN cDNA 2410002J21 gene |
| 1047 | 1.0710 | 1.3929 | 1.3312 | 1.3771 | 1.0304 | 12 | proteasome (prosome, macropain) subunit, alpha type 2 |
| 1048 | 1.2601 | 1.6010 | 1.5108 | 1.6820 | 1.1465 | 12 | guanosine monophosphate reductase |
| 1049 | 1.1352 | 1.7983 | 1.2672 | 1.5547 | 1.0281 | 12 | glutathione S-transferase, pi 2 |
| 1050 | 1.0400 | 1.4018 | 1.1995 | 1.3992 | 1.0924 | 12 | DNA methyltransferase 3B |
| 1051 | 1.0838 | 1.7832 | 1.3415 | 1.6079 | 1.1286 | 12 | major vault protein |
| 1052 | 0.9708 | 1.4280 | 1.2887 | 1.4485 | 1.3099 | 12 | craniofacial development protein 1 |
| 1053 | 0.9169 | 1.4190 | 1.2861 | 1.4841 | 1.2482 | 12 | SWI/SNF related, matrix associated, actin dependent regulator of |
| | | | | | | | chromatin, subfamily e, member 1 |
| 1054 | 0.9291 | 1.2736 | 1.2138 | 1.3638 | 1.1878 | 12 | eukaryotic translation initiation factor 3 |
| 1055 | 0.9989 | 1.7824 | 1.4076 | 1.8025 | 1.2388 | 12 | thioredoxin 1 |
| 1056 | 0.9763 | 1.4053 | 1.2160 | 1.3757 | 1.1421 | 12 | ESTs |
| 1057 | 0.9783 | 1.9044 | 1.5219 | 2.0547 | 1.2060 | 12 | mini chromosome maintenance deficient 7 (S. cerevisiae) |
| 1058 | 1.0135 | 1.3461 | 1.2286 | 1.3920 | 1.1570 | 12 | RIKEN cDNA 2600001N01 gene |
| 1059 | 1.1335 | 1.6446 | 1.4540 | 1.7949 | 1.3646 | 12 | Unknown |
| 1060 | 1.0333 | 1.5936 | 1.5660 | 1.8599 | 1.3577 | 12 | ribosomal protein L29 |
| 1061 | 1.0396 | 1.9237 | 1.7188 | 2.3890 | 1.4948 | 12 | ras homolog 9 (RhoC) |
| 1062 | 1.1069 | 2.1966 | 1.9482 | 2.6656 | 1.7530 | 12 | procollagen, type IV, alpha 1 |
| 1063 | 1.0399 | 1.6490 | 1.4289 | 1.6458 | 1.3296 | 12 | Mus musculus, clone IMAGE: 3494258, mRNA, partial cds |
| 1064 | 1.0548 | 1.2997 | 1.2611 | 1.3362 | 1.1771 | 12 | 5′,3′ nucleotidase, cytosolic |
| 1065 | 1.1342 | 1.3235 | 1.2802 | 1.3461 | 1.2371 | 12 | apoptosis inhibitory protein 5 |
| 1066 | 1.0484 | 1.3736 | 1.3444 | 1.4977 | 1.1073 | 12 | MYC-associated zinc finger protein (purine-binding transcription factor) |
| 1067 | 0.9670 | 1.4377 | 1.3039 | 1.4567 | 1.0584 | 12 | tyrosine 3-monooxygenase/tryptophan 5-monooxygenase activation protein, |
| | | | | | | | epsilon polypeptide |
| 1068 | 1.0794 | 1.9846 | 1.6828 | 2.0281 | 1.2816 | 12 | RIKEN cDNA 4930579A11 gene |
| 1069 | 1.0688 | 1.4107 | 1.3574 | 1.4379 | 1.1426 | 12 | Mus musculus, Similar to hypothetical protein DKFZp566A1524, clone |
| | | | | | | | MGC: 18989 IMAGE: 4012217, mRNA, complete cds |
| 1070 | 1.0884 | 1.7156 | 1.6628 | 1.7704 | 1.2620 | 12 | eukaryotic translation initiation factor 4E binding protein 1 |
| 1071 | 1.0272 | 1.6670 | 1.5489 | 1.7059 | 1.2080 | 12 | cardiac responsive adriamycin protein |
| 1072 | 1.0938 | 1.2541 | 1.2956 | 1.4300 | 1.1461 | 12 | procollagen lysine, 2-oxoglutarate 5-dioxygenase 2 |
| 1073 | 1.0534 | 1.2717 | 1.2368 | 1.3883 | 1.1354 | 12 | serine protease inhibitor, Kunitz type 2 |
| 1074 | 1.1051 | 1.2767 | 1.2656 | 1.3783 | 1.1899 | 12 | feline sarcoma oncogene |
| 1075 | 1.0318 | 1.6363 | 1.7177 | 2.0415 | 1.3129 | 12 | ribosomal protein S6 |
| 1076 | 1.0236 | 1.2391 | 1.2992 | 1.4582 | 1.1564 | 12 | cellular nucleic acid binding protein |
| 1077 | 0.7752 | 1.4606 | 0.9329 | 1.9073 | 1.2251 | 12 | arginase type II |
| 1078 | 0.8261 | 1.6489 | 1.0644 | 2.2978 | 1.3573 | 12 | procollagen, type IV, alpha 2 |
| 1079 | 1.0053 | 1.3440 | 1.1261 | 1.6085 | 1.1624 | 12 | cathepsin L |
| 1080 | 1.0803 | 1.2587 | 1.1580 | 1.3201 | 1.1786 | 12 | mitogen-activated protein kinase 7 |
| 1081 | 0.9961 | 1.3763 | 1.1463 | 1.3602 | 1.1504 | 12 | RIKEN cDNA 2700027J02 gene |
| 1082 | 1.1691 | 1.7019 | 1.2211 | 1.5698 | 1.3352 | 12 | integrin alpha 6 |
| 1083 | 0.7796 | 0.7212 | 0.7562 | 0.7826 | 0.5820 | 13 | RIKEN cDNA 1300013F15 gene |
| 1084 | 0.8123 | 0.8600 | 0.8336 | 0.8140 | 0.6928 | 13 | Cbp/p300-interacting transactivator with Glu/Asp-rich carboxy-terminal domain 1 |
| 1085 | 0.8480 | 0.9504 | 0.7898 | 0.7952 | 0.5793 | 13 | zinc finger like protein 1 |
| 1086 | 0.9117 | 1.0288 | 1.3129 | 0.9637 | 1.1158 | 14 | ubiquitin-like 1 |
| 1087 | 1.0415 | 1.2394 | 2.1900 | 1.3649 | 1.6645 | 14 | S100 calcium binding protein A4 |
| 1088 | 1.1017 | 1.1399 | 1.3869 | 1.1813 | 1.2344 | 14 | neutrophil cytosolic factor 2 |
| 1089 | 0.7711 | 1.2084 | 5.4112 | 1.5063 | 1.9326 | 14 | interferon activated gene 204 |
| 1090 | 1.0400 | 1.3497 | 1.7054 | 1.2355 | 1.2895 | 14 | RIKEN cDNA 5031412I06 gene |
| 1091 | 1.0369 | 1.1560 | 1.2849 | 1.1009 | 1.1142 | 14 | lectin, galactose binding, soluble 9 |
| 1092 | 1.0276 | 1.1616 | 1.3901 | 1.0470 | 1.1344 | 14 | clathrin, light polypeptide (Lca) |
| 1093 | 1.1597 | 1.3345 | 1.5498 | 1.2245 | 1.2991 | 14 | SEC61, gamma subunit (S. cerevisiae) |
| 1094 | 1.0055 | 1.1945 | 1.3226 | 1.1069 | 1.1835 | 14 | double cortin and calcium/calmodulin-dependent protein kinase-like 1 |
| 1095 | 0.9774 | 1.2138 | 1.6231 | 1.2368 | 1.2393 | 14 | reticulocalbin |
| 1096 | 0.9810 | 1.2926 | 1.7228 | 1.2361 | 1.4099 | 14 | Unknown |
| 1097 | 0.9880 | 1.1557 | 1.3407 | 1.1649 | 1.1829 | 14 | expressed sequence AW413625 |
| 1098 | 1.0192 | 1.3749 | 1.7257 | 1.3204 | 1.3531 | 14 | hematological and neurological expressed sequence 1 |
| 1099 | 0.9773 | 1.5072 | 2.0022 | 1.3664 | 1.5015 | 14 | epithelial membrane protein 3 |
| 1100 | 0.9348 | 1.2515 | 2.2390 | 1.0730 | 1.0296 | 14 | thymidine kinase 1 |
| 1101 | 1.0835 | 1.1962 | 1.7605 | 1.1520 | 1.1549 | 14 | RIKEN cDNA 1110038L14 gene |
| 1102 | 1.0410 | 1.0896 | 1.3744 | 1.0873 | 1.0573 | 14 | cathepsin Z |
| 1103 | 1.1411 | 1.2914 | 2.6723 | 1.5075 | 1.0320 | 14 | cell division cycle 2 homolog A (S. pombe) |
| 1104 | 1.1579 | 1.1821 | 1.6673 | 1.1931 | 1.0737 | 14 | CDC28 protein kinase 1 |
| 1105 | 0.9318 | 1.0360 | 1.8531 | 1.4314 | 1.2641 | 14 | expressed sequence AI449309 |
| 1106 | 1.1991 | 1.2134 | 1.5060 | 1.3744 | 1.2615 | 14 | bone marrow stromal cell antigen 1 |
| 1107 | 1.0601 | 1.2620 | 2.6800 | 1.7675 | 1.2322 | 14 | H2A histone family, member Z |
| 1108 | 0.9925 | 1.1426 | 3.4319 | 1.7880 | 1.1705 | 14 | leukemia-associated gene |
| 1109 | 1.0559 | 1.1309 | 1.2641 | 1.1876 | 1.0592 | 14 | ESTs, Weakly similar to limb expression 1 homolog (chicken) |
| | | | | | | | (Mus musculus) (M. musculus) |
| 1110 | 0.9930 | 1.1520 | 1.4468 | 1.3178 | 1.0507 | 14 | flap structure specific endonuclease 1 |
| 1111 | 0.9741 | 1.0881 | 1.2674 | 1.1409 | 1.0320 | 14 | RIKEN cDN 2010315L10 gene |
| 1112 | 0.9436 | 1.1237 | 1.2852 | 1.1427 | 1.0010 | 14 | latexin |
| 1113 | 0.8878 | 1.1129 | 1.3227 | 1.1430 | 1.0017 | 14 | integrin alpha M |
| 1114 | 0.9767 | 1.2741 | 2.0397 | 1.3380 | 1.1585 | 14 | high mobility group nucleosomal binding domain 2 |
| 1115 | 0.9003 | 1.0715 | 1.2528 | 1.1319 | 1.0338 | 14 | TEA domain family member 2 |
| 1116 | 1.0515 | 1.4555 | 2.3424 | 1.6998 | 1.4405 | 14 | platelet factor 4 |
| 1117 | 0.9140 | 1.1979 | 1.8263 | 1.3999 | 1.2170 | 14 | pyridoxal (pyridoxine, vitamin B6) kinase |
| 1118 | 0.9704 | 1.7875 | 1.2413 | 1.0728 | 1.3265 | 15 | A kinase (PRKA) anchor protein 2 |
| 1119 | 1.0255 | 1.8462 | 1.2927 | 1.1698 | 1.3029 | 15 | protein tyrosine phosphatase 4a1 |
| 1120 | 1.0495 | 1.3630 | 1.1613 | 1.0815 | 1.1375 | 15 | serine/arginine repetitive matrix 1 |
| 1121 | 0.9633 | 1.5063 | 1.3774 | 1.1703 | 1.5064 | 15 | CD2-associated protein |
| 1122 | 0.9473 | 1.2334 | 1.2088 | 1.0737 | 1.2287 | 15 | ESTs, Highly similar to prefoldin 4 (Homo sapiens) (H. sapiens) |
| 1123 | 0.9000 | 1.6154 | 1.3855 | 1.0621 | 1.2283 | 15 | interleukin 1 beta |
| 1124 | 1.0278 | 1.2534 | 1.1822 | 1.0738 | 1.1448 | 15 | Ras-GTPase-activating protein (GAP<120>) SH3-domain binding protein 2 |
| 1125 | 1.0268 | 1.4174 | 1.2491 | 1.1113 | 1.2210 | 15 | protein phosphatase 2a, catalytic subunit, beta isoform |
| 1126 | 1.0835 | 1.4000 | 1.2799 | 1.1386 | 1.2544 | 15 | mago-nashi homolog, proliferation-associated (Drosophila) |
| 1127 | 1.0188 | 1.1930 | 1.1787 | 1.0630 | 1.1650 | 15 | RIKEN cDNA 2610524G09 gene |
| 1128 | 0.9902 | 1.3364 | 1.2604 | 1.0297 | 1.2409 | 15 | microtubule-associated protein, RP/EB family, member 1 |
| 1129 | 0.9216 | 1.1940 | 1.1093 | 0.9664 | 1.1152 | 15 | RIKEN cDNA 1500026A19 gene |
| 1130 | 0.9093 | 1.3225 | 1.2436 | 0.9681 | 1.2763 | 15 | RIKEN cDNA 2810411G23 gene |
| 1131 | 0.9979 | 1.2759 | 1.1970 | 1.0145 | 1.2187 | 15 | serine/threonine kinase receptor associated protein |
| 1132 | 0.8501 | 1.3359 | 1.1779 | 1.0009 | 1.2015 | 15 | intergral membrane protein 1 |
| 1133 | 0.9389 | 1.3929 | 1.1776 | 1.0123 | 1.2458 | 15 | Unknown |
| 1134 | 1.0172 | 1.1777 | 1.2150 | 1.0220 | 1.1922 | 15 | CDC16 (cell division cycle 16 homolog (S. cerevisiae) |
| 1135 | 1.0058 | 1.1785 | 1.1752 | 0.9891 | 1.1682 | 15 | cornichon homolog (Drosophila) |
| 1136 | 1.0015 | 1.2492 | 1.1454 | 1.0197 | 1.3606 | 15 | homeo box B7 |
| 1137 | 0.9485 | 1.1812 | 1.1673 | 0.9851 | 1.2455 | 15 | methionine aminopeptidase 2 |
| 1138 | 0.9893 | 1.1928 | 1.1357 | 0.9582 | 1.2270 | 15 | poliovirus receptor-related 3 |
| 1139 | 0.8686 | 0.7475 | 0.7194 | 0.8121 | 0.9798 | 16 | ESTs |
| 1140 | 0.9742 | 0.8250 | 0.8360 | 0.9492 | 1.1294 | 16 | eukaryotic translation initiation factor 4A2 |
| 1141 | 0.9773 | 0.8609 | 0.8524 | 0.9391 | 1.0958 | 16 | Unknown |
| 1142 | 1.0484 | 0.8604 | 0.8549 | 0.9882 | 1.2306 | 16 | expressed sequence C85457 |
| 1143 | 0.9603 | 0.8090 | 0.8159 | 1.0539 | 1.1504 | 16 | expressed sequence AI465301 |
| 1144 | 0.9671 | 0.8303 | 0.8069 | 1.0288 | 1.1462 | 16 | RIKEN cDNA 1200003E16 gene |
| 1145 | 1.1326 | 1.0243 | 0.9914 | 1.1795 | 1.2983 | 16 | RIKEN cDNA 473340LN12 gene |
| 1146 | 0.7944 | 0.7365 | 0.6909 | 0.8202 | 0.9165 | 16 | expressed sequence AA672638 |
| 1147 | 0.9335 | 0.8055 | 0.7684 | 0.9555 | 1.1355 | 16 | expressed sequence AI558103 |
| 1148 | 0.9951 | 0.8270 | 0.8153 | 1.0046 | 1.2762 | 16 | RIKEN cDNA 1100001J13 gene |
| 1149 | 1.0462 | 0.8143 | 0.7505 | 1.1385 | 1.1028 | 16 | calsyntenin 1 |
| 1150 | 0.9734 | 0.8666 | 0.8087 | 1.0095 | 1.0230 | 16 | topoisomerase (DNA) III beta |
| 1151 | 0.9391 | 0.8452 | 0.7843 | 1.0588 | 1.0228 | 16 | Mus musculus, Similar to sirtuin silent mating type information regulation 2 |
| | | | | | | | homolog 7 (S. cerevisiae), clone MGC: 37560 IMAGE: 4987746, mRNA, |
| | | | | | | | complete cds |
| 1152 | 0.9457 | 0.7893 | 0.6889 | 1.0771 | 1.1442 | 16 | anterior gradient 2 (Xenopus laevis) |
| 1153 | 0.9818 | 0.8115 | 0.7371 | 1.0933 | 1.1716 | 16 | expressed sequence C86169 |
| 1154 | 0.8276 | 0.6977 | 0.6375 | 0.8955 | 0.9746 | 16 | RIKEN cDNA A930008K15 gene |
| 1155 | 0.9242 | 0.8591 | 0.7774 | 0.9837 | 1.0225 | 16 | ESTs |
| 1156 | 0.8480 | 0.7853 | 0.7231 | 0.9216 | 0.9329 | 16 | vascular endothelial growth factor A |
| 1157 | 0.5563 | 0.4769 | 0.3989 | 0.6646 | 0.6648 | 16 | Mus musculus, clone MGC: 36388 IMAGE: 5098924, mRNA, complete cds |
| 1158 | 0.8253 | 0.7608 | 0.6957 | 0.7984 | 0.8143 | 16 | Mus musculus LDLR dan mRNA, complete cds |
| 1159 | 0.9553 | 0.7901 | 0.7037 | 0.9032 | 0.9327 | 16 | Mus musculus, Similar to hypothetical protein FLJ12618, clone MGC: 28775 |
| | | | | | | | IMAGE: 4487011, mRNA, complete cds |
| 1160 | 1.0320 | 0.8286 | 0.7437 | 0.9322 | 0.9812 | 16 | ceroid-lipofuscinosis, neuronal 2 |
| 1161 | 0.9159 | 0.5710 | 0.5189 | 0.7486 | 0.8705 | 16 | insulin-like growth factor binding protein 3 |
| 1162 | 0.9547 | 0.5214 | 0.4517 | 0.7660 | 0.8212 | 16 | fatty acid synthase |
| 1163 | 1.1278 | 0.6844 | 0.5641 | 0.9455 | 0.9828 | 16 | glycine N-methyltransferase |
| 1164 | 1.0041 | 0.7513 | 0.7156 | 0.9468 | 0.9649 | 16 | sphingomyelin phosphodiesterase 2, neutral |
| 1165 | 1.1925 | 0.8881 | 0.8160 | 1.1213 | 1.1124 | 16 | expressed sequence AI413466 |
| 1166 | 0.9753 | 0.8457 | 0.7352 | 0.9649 | 1.0476 | 16 | EGL nine homolog 1 (C. elegans) |
| 1167 | 0.9118 | 0.8582 | 0.7986 | 0.8836 | 0.9247 | 16 | RIKEN cDNA A230106A15 gene |
| 1168 | 1.0686 | 0.8894 | 0.8360 | 0.9758 | 1.1393 | 16 | ESTs, Weakly similar to ADT1 MOUSE ADP, ATP CARRIER PROTEIN, |
| | | | | | | | HEART/SKELETAL MUSCLE ISOFORM T1 (M. musculus) |
| 1169 | 0.9471 | 0.8392 | 0.7884 | 0.9496 | 1.0455 | 16 | osteomodulin |
| 1170 | 0.8930 | 0.6485 | 0.5872 | 0.8122 | 0.9619 | 16 | solute carrier family 15 (H+/peptide transporter), member 2 |
| 1171 | 1.0457 | 0.8996 | 0.8571 | 1.0381 | 1.1017 | 16 | protein phosphatase 3, catalytic subunit, gamma isoform |
| 1172 | 1.0633 | 0.9249 | 0.8695 | 1.0370 | 1.1045 | 16 | serine palmitoyltransferase, long chain base subunit 1 |
| 1173 | 0.9216 | 0.6808 | 0.7463 | 1.0223 | 0.9112 | 16 | G protein-coupled receptor kinase 7 |
| 1174 | 0.9487 | 0.7324 | 0.7956 | 0.9837 | 0.9209 | 16 | expressed sequence AI265322 |
| 1175 | 0.9495 | 0.6557 | 0.7324 | 1.0143 | 0.8905 | 16 | solute carrier family 16 (monocarboxylic acid transporters), member 2 |
| 1176 | 1.0473 | 0.6975 | 0.8004 | 1.1131 | 0.9743 | 16 | ESTs, Weakly similar to brain-specific angiogenesis inhibitor 1-associated protein 2 |
| | | | | | | | (Mus musculus) (M. musculus) |
| 1177 | 1.0189 | 0.5147 | 0.5892 | 0.8992 | 0.8150 | 16 | junction plakoglobin |
| 1178 | 1.0214 | 0.8563 | 0.8755 | 1.0146 | 0.9805 | 16 | RIKEN cDNA 1010001J06 gene |
| 1179 | 0.9818 | 0.8350 | 0.8525 | 0.9649 | 0.9412 | 16 | solute carrier family 31, member 1 |
| 1180 | 1.0867 | 0.8276 | 0.8304 | 1.2240 | 0.9849 | 16 | Unknown |
| 1181 | 0.9647 | 0.8596 | 0.8314 | 1.0452 | 0.9370 | 16 | Mus musculus, Similar to 60S ribosomal protein L30 isolog, clone MGC: 6735 |
| | | | | | | | IMAGE: 3590401, mRNA, complete cds |
| 1182 | 1.0488 | 0.7387 | 0.7588 | 1.0728 | 0.9178 | 16 | ESTs, Highly similar to T00268 hypothetical protein KIAA0597 (H. sapiens) |
| 1183 | 0.9630 | 0.7481 | 0.7436 | 1.0938 | 0.9276 | 16 | RIKEN cDNA A330103N21 gene |
| 1184 | 1.0471 | 0.8715 | 0.8655 | 1.0884 | 1.0194 | 16 | ESTs |
| 1185 | 1.0434 | 0.8567 | 0.8687 | 1.1050 | 1.0021 | 16 | Rho guanine nucleotide exchange factor (GEF) 3 |
| 1186 | 0.9598 | 0.7986 | 0.7870 | 1.0067 | 0.9204 | 16 | Mus musculus, clone MGC: 38798 IMAGE: 5359803, mRNA, complete cds |
| 1187 | 1.1232 | 0.7923 | 0.7875 | 1.2412 | 1.0434 | 16 | RIKEN cDNA 0610011C19 gene |
| 1188 | 1.0499 | 0.8278 | 0.8049 | 1.0587 | 1.0152 | 16 | growth factor receptor bound protein 7 |
| 1189 | 0.9439 | 0.8329 | 0.8138 | 0.9656 | 0.9398 | 16 | phospholipase A2, group IIA (platelets, synovial fluid) |
| 1190 | 1.0047 | 0.8441 | 0.7703 | 1.0302 | 0.9481 | 16 | ESTs |
| 1191 | 1.0120 | 0.8037 | 0.7487 | 1.0356 | 0.8979 | 16 | hexokinase 1 |
| 1192 | 1.0384 | 0.9324 | 0.9168 | 1.0256 | 0.9948 | 16 | RIKEN cDNA 2310010G13 gene |
| 1193 | 0.9873 | 0.8435 | 0.8001 | 0.9836 | 0.9220 | 16 | alpha-methylacyl-CoA racemase |
| 1194 | 1.0463 | 0.6703 | 0.8228 | 1.1699 | 1.1217 | 16 | golgi autoantigen, golgin subfamily a, 4 |
| 1195 | 0.8462 | 0.4888 | 0.6832 | 1.0029 | 0.9328 | 16 | cytochrome P450, 2e1, ethanol inducible |
| 1196 | 1.1521 | 0.9298 | 0.9929 | 1.3168 | 1.2586 | 16 | expressed sequence AI316828 |
| 1197 | 0.9514 | 0.7845 | 0.8686 | 1.0294 | 1.0380 | 16 | centrin 2 |
| 1198 | 1.2042 | 1.0528 | 1.1342 | 1.3108 | 1.3002 | 16 | RIKEN cDNA 5730406I15 gene |
| 1199 | 1.1674 | 1.0414 | 1.0408 | 1.3230 | 1.2427 | 16 | nuclear receptor subfamily 2, group F, member 6 |
| 1200 | 0.9744 | 0.8216 | 0.8173 | 1.0253 | 0.9929 | 16 | peroxisomal biogenesis factor 13 |
| 1201 | 0.9459 | 0.8702 | 0.8721 | 0.9801 | 0.9593 | 16 | expressed sequence AW552393 |
| 1202 | 0.9986 | 0.8072 | 0.8296 | 1.0988 | 1.0155 | 16 | erythrocyte protein band 4.1-like 1 |
| 1203 | 1.0713 | 0.8327 | 0.8878 | 1.2049 | 1.1226 | 16 | ESTs, Weakly similar to S26689 hypothetical protein hc1 - mouse (M. musculus) |
| 1204 | 0.9048 | 0.7019 | 0.7891 | 0.9459 | 1.1862 | 16 | CD59a antigen |
| 1205 | 0.8098 | 0.5689 | 0.6880 | 0.9742 | 1.1281 | 16 | tetranectin (plasminogen binding protein) |
| 1206 | 0.8417 | 0.5339 | 0.6417 | 0.8740 | 0.9940 | 16 | stromal cell derived factor 1 |
| 1207 | 0.9219 | 0.7310 | 0.8274 | 0.9510 | 1.0110 | 16 | ESTs |
| 1208 | 0.9231 | 0.6366 | 0.7259 | 0.9127 | 0.9244 | 16 | pre B-cell leukemia transcription factor 1 |
| 1209 | 0.7930 | 0.4267 | 0.5527 | 0.6626 | 0.8417 | 16 | low density lipoprotein receptor-related protein 2 |
| 1210 | 0.8084 | 0.5246 | 0.6091 | 0.7451 | 0.8629 | 16 | endonuclease G |
| 1211 | 1.0220 | 0.7353 | 0.8341 | 0.9693 | 1.1231 | 16 | transmembrane 7 superfamily member 1 |
| 1212 | 0.8718 | 0.6501 | 0.6681 | 0.8363 | 0.8854 | 16 | Williams-Beuren syndrome chromosome region 14 homolog (human) |
| 1213 | 0.8370 | 0.6306 | 0.6710 | 0.8035 | 0.8692 | 16 | RIKEN cDNA 2610524G07 gene |
| 1214 | 0.9220 | 0.6816 | 0.7257 | 0.8975 | 0.9515 | 16 | expressed sequence AI553555 |
| 1215 | 1.0362 | 0.5204 | 0.6464 | 0.8903 | 1.0545 | 16 | calpain, small subunit 1 |
| 1216 | 1.0469 | 0.6953 | 0.7449 | 0.9300 | 1.0651 | 16 | expressed sequence AI838057 |
| 1217 | 0.9002 | 0.5735 | 0.6361 | 0.7924 | 0.9450 | 16 | vitamin D receptor |
| 1218 | 0.7460 | 0.6187 | 0.6259 | 0.8153 | 0.8373 | 16 | RIKEN cDNA A330103N21 gene |
| 1219 | 1.0014 | 0.7697 | 0.7718 | 0.9483 | 1.0921 | 16 | PH domain containing protein in retina 1 |
| 1220 | 0.8994 | 0.6916 | 0.7194 | 0.9090 | 0.9422 | 16 | insulin-like growth factor binding protein, acid labile subunit |
| 1221 | 0.9126 | 0.7771 | 0.7863 | 0.9175 | 0.9253 | 16 | Mus musculus, clone IMAGE: 3155544, mRNA, partial cds |
| 1222 | 1.0124 | 0.7874 | 0.7765 | 0.9927 | 1.0544 | 16 | RIKEN cDNA 2610039E05 gene |
| 1223 | 1.1773 | 0.9770 | 0.9666 | 1.1599 | 1.2159 | 16 | RIKEN cDNA 2810468K17 gene |
| 1224 | 1.0799 | 0.7978 | 0.8182 | 1.0755 | 1.1785 | 16 | ras homolog gene family, member E |
| 1225 | 1.0972 | 0.8667 | 0.8683 | 1.1284 | 1.1788 | 16 | RIKEN cDNA 1110004G16 gene |
| 1226 | 0.7216 | 0.4264 | 0.5756 | 0.6703 | 0.9598 | 17 | amine N-sulfotransferase |
| 1227 | 0.9077 | 0.6041 | 0.7360 | 0.7749 | 0.9896 | 17 | slit homolog 2 (Drosophila) |
| 1228 | 0.8697 | 0.6488 | 0.7532 | 0.7733 | 0.9789 | 17 | acetyl-Coenzyme A transporter |
| 1229 | 0.8753 | 0.7897 | 0.7380 | 0.7660 | 0.9231 | 17 | expressed sequence AI528491 |
| 1230 | 0.9602 | 0.7748 | 0.8252 | 0.7941 | 1.0085 | 17 | thiamin pyrophosphokinase |
| 1231 | 0.7704 | 0.6657 | 0.6761 | 0.6868 | 0.8250 | 17 | kynureninase (L-kynurenine hydrolase) |
| 1232 | 0.9486 | 0.9472 | 0.6684 | 0.6223 | 0.6833 | 18 | RIKEN cDNA 0610006F02 gene |
| 1233 | 0.7284 | 0.7072 | 0.5282 | 0.4953 | 0.4893 | 18 | acyl-Coenzyme A oxidase 1, palmitoyl |
| 1234 | 0.8229 | 0.8975 | 0.5174 | 0.5960 | 0.6500 | 18 | solute carrier family 22 (organic anion transporter), member 6 |
| 1235 | 0.9488 | 0.9660 | 0.7584 | 0.7499 | 0.8044 | 18 | thioredoxin 2 |
| 1236 | 1.0921 | 1.2183 | 0.6037 | 0.6404 | 0.7653 | 18 | glutathione S-transferase, alpha 2 (Yc2) |
| 1237 | 0.8047 | 1.2541 | 0.7664 | 0.6000 | 0.7622 | 18 | heat shock protein, 60 kDa |
| 1238 | 0.7910 | 1.0130 | 0.6932 | 0.6256 | 0.7714 | 18 | glycerol phosphate dehydrogenase 1, mitochondrial |
| 1239 | 0.6354 | 0.7465 | 0.5635 | 0.5398 | 0.5464 | 18 | FK506 binding protein 5 (51 kDa) |
| 1240 | 0.8518 | 0.9328 | 0.6746 | 0.5655 | 0.7288 | 18 | ESTs |
| 1241 | 1.0175 | 1.1026 | 0.8252 | 0.6950 | 0.8350 | 18 | X transporter protein 2 |
| 1242 | 0.9132 | 0.9692 | 0.7408 | 0.6057 | 0.6761 | 18 | reduced expression 3 |
| 1243 | 0.6794 | 0.8598 | 0.5417 | 0.3830 | 0.5091 | 18 | cytochrome P450, subfamily IV B, polypeptide 1 |
| 1244 | 0.9882 | 1.1147 | 0.8788 | 0.7661 | 0.8372 | 18 | M. musculus mRNA for protein expressed at high levels in testis |
| 1245 | 0.9341 | 0.9366 | 1.0583 | 0.7853 | 0.7892 | 19 | expressed sequence AI646725 |
| 1246 | 1.0022 | 1.0738 | 1.1943 | 0.9493 | 0.9383 | 19 | expressed sequence AI461788 |
| 1247 | 1.0895 | 1.2456 | 1.4707 | 0.9443 | 0.9587 | 19 | expressed in non-metastatic cells 2, protein (NM23B) (nucleoside |
| | | | | | | | diphosphate kinase) |
| 1248 | 1.0315 | 1.1499 | 1.3408 | 0.9272 | 0.9469 | 19 | hyaluronan mediated motility receptor (RHAMM) |
| 1249 | 1.0735 | 1.1506 | 1.4151 | 1.0051 | 0.9070 | 19 | ESTs |
| 1250 | 1.1030 | 1.2784 | 1.5842 | 0.9665 | 0.8870 | 19 | activator of S phase kinase |
| 1251 | 0.9655 | 0.9903 | 1.1716 | 0.7785 | 0.5639 | 19 | Unknown |
| 1252 | 0.9137 | 0.9440 | 0.9868 | 0.8497 | 0.7866 | 19 | RIKEN cDNA 1700008H23 gene |
| 1253 | 1.0341 | 1.1379 | 1.1618 | 1.0010 | 0.8596 | 19 | glycine transporter 1 |
| 1254 | 1.0317 | 1.1435 | 1.1596 | 0.9721 | 0.8924 | 19 | RIKEN cDNA 1700037H04 gene |
| 1255 | 1.0455 | 1.2064 | 1.1684 | 0.9953 | 0.8952 | 19 | cell division cycle 25 homolog A (S. cerevisiae) |
| 1256 | 1.0634 | 1.2368 | 1.2412 | 1.0252 | 0.9125 | 19 | ESTs, Weakly similar to T29029 hypothetical protein F53G12.5 - |
| | | | | | | | Caenorhabditis elegans (C. elegans) |
| 1257 | 0.9991 | 1.1573 | 1.1333 | 0.9716 | 0.8894 | 19 | serine (or cysteine) proteinase inhibitor, clade B (ovalbumin), member 2 |
| 1258 | 0.8331 | 1.1946 | 1.1676 | 0.7561 | 0.6011 | 19 | ESTs |
| 1259 | 1.0370 | 1.1831 | 1.3056 | 0.5446 | 0.5428 | 19 | Mus musculus mRNA for 67 kDa polymerase-associated factor PAF67 (paf67 gene) |
| 1260 | 0.9926 | 1.0357 | 1.0922 | 0.7913 | 0.8043 | 19 | ESTs |
| 1261 | 1.0820 | 1.0922 | 1.1351 | 0.7153 | 0.6720 | 19 | renin 2 tandem duplication of Ren1 |
| 1262 | 0.8256 | 0.8637 | 0.8636 | 0.6261 | 0.6215 | 19 | Mus musculus, clone MGC: 18871 IMAGE: 4234793, mRNA, complete cds |
| 1263 | 1.0303 | 1.0384 | 1.0400 | 0.7633 | 0.7602 | 19 | ESTs |
| 1264 | 0.8423 | 0.8741 | 0.8496 | 0.7975 | 0.8076 | 19 | methyl CpG binding protein 2 |
| 1265 | 1.1232 | 1.2488 | 1.2476 | 0.9984 | 1.0648 | 19 | translin |
| 1266 | 1.1191 | 1.4030 | 1.4152 | 0.9465 | 0.9676 | 19 | RNA polymerase I associated factor, 53 kD |
| 1267 | 0.9354 | 1.3279 | 1.3514 | 0.8133 | 0.7730 | 19 | glutathione peroxidase 1 |
| 1268 | 1.1413 | 1.2589 | 1.2178 | 1.0664 | 1.0569 | 19 | expressed sequence AI450991 |
| 1269 | 0.9943 | 1.6060 | 1.5081 | 0.5737 | 0.5794 | 19 | inosine 5′-phosphate dehydrogenase 2 |
| 1270 | 1.0331 | 1.3788 | 1.2981 | 0.7631 | 0.8181 | 19 | ornithine decarboxylase, structural |
| 1271 | 0.9425 | 0.7462 | 0.6442 | 0.8395 | 0.6508 | 20 | expressed sequence AI957255 |
| 1272 | 0.9854 | 0.6898 | 0.6696 | 0.8035 | 0.6520 | 20 | carnitine palmitoyltransferase 2 |
| 1273 | 0.7782 | 0.6941 | 0.6735 | 0.7359 | 0.6717 | 20 | RIKEN cDNA 2900074L19 gene |
| 1274 | 1.0423 | 0.7542 | 0.8140 | 0.9884 | 0.7076 | 20 | expressed sequence AI852479 |
| 1275 | 0.9971 | 0.8408 | 0.8286 | 0.9739 | 0.7318 | 20 | Mus musculus adult male kidney cDNA, RIKEN full-length enriched library, |
| | | | | | | | clone:0610012C11:homogentisate 1,2-dioxygenase, full insert sequence |
| 1276 | 1.0314 | 0.9477 | 0.9294 | 1.0643 | 0.8907 | 20 | expressed sequence AI848669 |
| 1277 | 0.6297 | 0.6638 | 0.5796 | 0.7164 | 0.5609 | 21 | period homolog 2 (Drosophila) |
| 1278 | 1.2346 | 1.2863 | 1.1960 | 1.3450 | 1.2365 | 21 | AMP deaminase 3 |
| 1279 | 1.1882 | 1.2699 | 1.5683 | 0.9345 | 1.1416 | 22 | ESTs |
| 1280 | 1.0289 | 1.0948 | 1.1865 | 1.0073 | 1.0780 | 22 | RIKEN cDNA 2700099C19 gene |
| 1281 | 1.0470 | 1.0996 | 1.3180 | 1.0282 | 1.1319 | 22 | FK506 binding protein 9 |
| 1282 | 0.8497 | 0.6632 | 0.8776 | 0.6948 | 0.7460 | 23 | selenophosphate synthetase 2 |
| 1283 | 0.7892 | 0.6607 | 0.8061 | 0.6815 | 0.6965 | 23 | prion protein |
| 1284 | 0.9053 | 0.4449 | 0.8809 | 0.5662 | 0.5433 | 23 | NADPH oxidase 4 |
| 1285 | 1.0404 | 0.5635 | 1.1677 | 0.7781 | 0.7421 | 23 | 2-hydroxyphytanoyl-CoA lyase |
| 1286 | 0.8847 | 0.7504 | 0.9388 | 0.7553 | 0.7135 | 23 | four and a half LIM domains 1 |
| 1287 | 0.9811 | 0.7886 | 0.9567 | 0.7790 | 0.7844 | 23 | hyaluronic acid binding protein 2 |
| 1288 | 1.2003 | 1.0812 | 1.2603 | 1.3362 | 1.3216 | 24 | transcription factor Dp 1 |
| 1289 | 1.2993 | 1.0302 | 1.2843 | 1.3916 | 1.4033 | 24 | ESTs, Weakly similar to JE0096 myocilin - mouse (M. musculus) |
| 1290 | 1.0760 | 0.8888 | 1.1042 | 1.2025 | 1.2461 | 24 | retinoblastoma binding protein 4 |
| 1291 | 1.0583 | 1.1440 | 1.2454 | 1.0365 | 1.3095 | 25 | Mus musculus, Similar to RAS p21 protein activator, clone MGC: 7759 |
| | | | | | | | IMAGE: 3498774, mRNA, complete cds |
| 1292 | 0.7509 | 0.7341 | 0.5281 | 0.6906 | 0.8522 | 26 | RIKEN cDNA 1700012B18 gene |
| 1293 | 0.7475 | 0.7636 | 0.7379 | 0.6815 | 0.7817 | 27 | Mus musculus, Similar to angiopoietin-like factor, clone MGC: 32448 IMAGE: |
| | | | | | | | 5043159, mRNA, complete cds |

TABLE 15
The RRR 1325 genes expression data and specific functional gene-clusters,
1325 unique genes were identified in the current microarray dataset. The gene expression is
presented as up or down from normal-ischemic kidneys. Two separate groups of microarray
experiments were conducted, and the results were subsequently normalized to eliminate
systematic bias. The first group consisted of normal and ischemic tissues, as well as and 1 and
2 days post-injury. The second group consisted of normal kidneys and 5 and 14 days post-
injury. The data from days 1 and 2 were normalized by the mean of the normal-ischemic
group, and the data from days 5 and 14 by the mean of the corresponding normal kidney. The
genes were further clustered according to RCC vs. normal kidney; renal cell culture hypoxia
responsive genes vs. normoxia; HIF regulated genes; VHL, IGF1, MYC, NF-□B pathway
genes; purine pathway genes; gene expression following renal ischemia reperfusion and/or
acute renal failure (ARF) vs. normal tissue; and gene expression in response to serum (1, 2).

| | | Time | |
|---|---|---|---|
| | | points: | |
| | | Early (A); | |
| | | Late (B); | |
| | | Early | p-value |
| | | & late (*) | (days 1-2 vs |
| | | changed | Normal- |
| Gene name | Symbol Human | gene | Ischemic) |
| (Gus-s) beta-glucuronidase structural | GUSB | b | |
| (Prlr-rs1) prolactin receptor related sequence 1 | PRLR | * | 0.0005 |
| (Sdccagg28) serologically defined colon cancer antigen 28 | STARD10 | a | 0.0012 |
| ((AW146109) expressed sequence AW146109) | CD44 | * | 0.0018 |
| (2610524K04Rik; RIKEN cD 2610524K04 gene) | pp90RSK4 | a | 0.0013 |
| 1-acylglycerol-3-phosphate O-acyltransferase 3; expressed | AGPAT3 | a | 0.0042 |
| sequence AW493985 | | | |
| 2′-5′ oligoadenylate synthetase 1A | OAS1 | a | 0.0202 |
| 2-hydroxyphytanoyl-CoA lyase | HPCL2 | b | |
| 3-hydroxy-3-methylglutaryl-Coenzyme A synthase 1 | HMGCS1 | a | 0.0011 |
| 3-phosphoglycerate dehydrogese | PHGDH | a | 0.0018 |
| 4-hydroxyphenylpyruvic acid dioxygese | HPD | * | 0.0005 |
| 5′,3′ nucleotidase, cytosolic | NT5C | b | |
| 5-azacytidine induced gene 1 | Azi1 | a | 0.0079 |
| a disintegrin and metalloproteise domain 12 (meltrin alpha) | ADAM12 | * | 0.019 |
| a disintegrin-like and metalloprotease (reprolysin type) with | ADAMTS1 | * | 0.0005 |
| thrombospondin type 1 motif, 1 | | | |
| a disintegrin-like and metalloprotease (reprolysin type) with | ADAMTS2 | a | 0.0347 |
| thrombospondin type 1 motif, 2 | | | |
| A kise (PRKA) anchor protein 2 | AKAP2 | a | 0.0215 |
| acetyl-Coenzyme A acyltransferase 2 (mitochondrial 3- | ACAA2 | * | 0.0006 |
| oxoacyl-Coenzyme A thiolase) (D18Ertd240e) RIKEN cD | | | |
| 0610011L04 gene | | | |
| acetyl-Coenzyme A dehydrogese, medium chain | ACADM | a | 0.0005 |
| acetyl-Coenzyme A transporter | ACATN | a | 0.0064 |
| acidic ribosomal phosphoprotein PO | RPLP0 | a | 0.0006 |
| aconitase 1 | ACO1 | b | |
| actin related protein ⅔ complex, subunit 3 (21 kDa) | ARPC3 | a | 0.0023 |
| actin, alpha 1, skeletal muscle | ACTA1 | b | |
| actin, alpha 2, smooth muscle, aorta | ACTA2 | * | 0.0005 |
| actin, beta, cytoplasmic | ACTB | * | 0.0005 |
| actin, gamma 2, smooth muscle, enteric | ACTG2 | * | 0.013 |
| actin-like | ACTG1 | * | 0.0005 |
| activator of S phase kise | ASK | a | 0.0283 |
| activity-dependent neuroprotective protein | ADNP | b | |
| acyl-Coenzyme A dehydrogese, short/branched chain | ACADSB | * | 0.0245 |
| acyl-Coenzyme A dehydrogese, very long chain | ACADVL | b | |
| acyl-Coenzyme A oxidase 1, palmitoyl | ACOX1 | b | |
| adaptor-related protein complex AP-3, sigma 1 subunit | AP3S1 | a | 0.0109 |
| adducin 3 (gamma) | ADD3 | b | |
| adenine phosphoribosyl transferase | APRT | b | |
| adenylate cyclase 4 | ADCY4 | a | 0.0472 |
| adenylate kise 4 | Ak4 | * | 0.0008 |
| adenylosuccite synthetase 2, non muscle | ADSS | (a + b) = * | 0.004 |
| adenylyl cyclase-associated CAP protein homolog 1 (S. cerevisiae, | CAP | a | 0.0127 |
| S. pombe) | | | |
| ADP-ribosylation factor 1 | ARF1 | a | 0.0012 |
| ADP-ribosyltransferase (D+ | ADPRTL2 | a | 0.003 |
| AE binding protein 1 | AEBP1 | b | |
| ajuba | JUB | b | |
| alcohol dehydrogese 4 (class II), pi polypeptide | ADH4 | b | |
| aldehyde dehydrogese family 1, subfamily A2 | ALDH1A2 | b | |
| aldo-keto reductase family 1, member B8 ((Fgfrp) fibroblast | AKR1B10 | * | 0.0016 |
| growth factor regulated protein) | | | |
| aldo-keto reductase family 1, member C18; expressed | Akr1c18 | a | 0.0025 |
| sequence AW146047 | | | |
| alkaline phosphatase 2, liver | ALPL | a | 0.0096 |
| ALL1-fused gene from chromosome 1q | AF1Q | a | 0.0049 |
| alpha-methylacyl-CoA racemase | AMACR | a | 0.0472 |
| amelogenin | AMELX | b | |
| amiloride binding protein 1 (amine oxidase, copper-containing) | ABP1 | * | 0.005 |
| amine N-sulfotransferase | Sultn | a | 0.0472 |
| aminoadipate-semialdehyde synthase/(Lorsdh) lysine | AASS | * | 0.0008 |
| oxoglutarate reductase, saccharopine dehydrogese | | | |
| AMP deamise 3 | AMPD3 | b | |
| annexin A1 | ANXA1 | b | |
| annexin A2 | ANXA2 | * | 0.0005 |
| annexin A3 | ANXA3 | b | |
| annexin A4 | ANXA4 | b | |
| annexin A5 | ANXA5 | * | 0.0005 |
| annexin A6 | ANXA6 | * | 0.0005 |
| anterior gradient 2 (Xenopus laevis) | AGR2 | a | 0.0044 |
| apolipoprotein B editing complex 1 | APOBEC1 | b | |
| apolipoprotein E | APOE | b | |
| apoptosis inhibitory protein 5 | API5 | b | |
| apurinic/apyrimidinic endonuclease | APEX1 | a | 0.0005 |
| aquaporin 2 | AQP2 | a | 0.0027 |
| arachidote 12-lipoxygese, pseudogene 2 | ALOX12P2 | b | |
| arachidote 5-lipoxygese activating protein | ALOX5AP | a | 0.0135 |
| arginine-rich, mutated in early stage tumors | ARMET | a | 0.0013 |
| argise type II | ARG2 | b | |
| Arpc2 | ARPC2 | * | 0.0005 |
| ATP synthase, H+ transporting mitochondrial F1 complex, beta | ATP5B | a | 0.0081 |
| subunit | | | |
| ATP synthase, H+ transporting, mitochondrial F1 complex, | ATP5A1 | a | 0.0035 |
| alpha subunit, isoform 1 | | | |
| ATPase, +/K+ transporting, beta 1 polypeptide | ATP1B1 | b | |
| ATPase, H+ transporting, lysosomal (vacuolar proton pump), | ATP6V1A1 | a | 0.0269 |
| alpha 70 kDa, isoform 1 | | | |
| ATPase, H+ transporting, V1 subunit F; RIKEN cD | ATP6V1F | a | 0.0028 |
| 1110004G16 gene | | | |
| ATPase, H+/K+ transporting, alpha polypeptide | ATP4A | a | 0.0231 |
| ATP-binding cassette, sub-family A (ABC1), member 7 | ABCA7 | b | |
| ATP-binding cassette, sub-family D (ALD), member 3 | ABCD3 | * | 0.0193 |
| AU R binding protein/enoyl-coenzyme A hydratase | AUH | * | 0.0012 |
| avian reticuloendotheliosis viral (v-rel) oncogene related B | RELB | b | |
| AXL receptor tyrosine kise | AXL | * | 0.0005 |
| baculoviral IAP repeat-containing 1a | BIRC1 | * | 0.0017 |
| baculoviral IAP repeat-containing 2 | BIRC3 | b | |
| baculoviral IAP repeat-containing 3 | BIRC3 | b | |
| B-box and SPRY domain containing | BSPRY | b | |
| B-cell leukemia/lymphoma 2 related protein A1b | BCL2A1 | * | 0.0034 |
| BCL2-antagonist/killer 1 | BAK1 | b | |
| Bcl-2-related ovarian killer protein | BOK | b | |
| benzodiazepine receptor, peripheral | BZRP | b | |
| beta-2 microglobulin | B2M | b | |
| betaine-homocysteine methyltransferase | BHMT | a | 0.0005 |
| biglycan | BGN | * | 0.0219 |
| bisphosphate 3′-nucleotidase 1 | BPNT1 | b | |
| Blu protein | ZMYND10 | a | 0.0042 |
| bone marrow stromal cell antigen 1 | BST1 | * | 0.03 |
| bone morphogenetic protein receptor, type 1A | BMPR1A | b | |
| brain protein 44-like | BRP441 | a | 0.0005 |
| branched chain aminotransferase 2, mitochondrial | BCAT2 | a | 0.0005 |
| branched chain ketoacid dehydrogese E1, alpha polypeptide | BCKDHA | * | 0.0005 |
| breakpoint cluster region protein 1 | BANF1 | a | 0.0005 |
| BRG1/brm-associated factor 53A | BAF53A | * | 0.0482 |
| Bromodomain and PHD finger containing, 3 | Brpf3 | a | 0.0115 |
| cadherin 3 | CDH3 | * | 0.0041 |
| calbindin-28K | CALB1 | * | 0.0005 |
| calbindin-D9K | CALB3 | a | 0.0086 |
| calcium channel, voltage-dependent, beta 3 subunit | CACNB3 | b | |
| calpain 2 | CAPN2 | b | |
| calpain, small subunit 1 | CAPNS1 | a | 0.0013 |
| calponin 2 | CNN2 | * | 0.0018 |
| calreticulin | CALR | a | 0.0238 |
| calsyntenin 1 | CLSTN1 | a | 0.0068 |
| capping protein beta 1 | CAPZB | * | 0.0043 |
| carbonic anhydrase 5a, mitochondrial | CA5A | a | 0.0478 |
| carboxylesterase 3 | CES3 | * | 0.0031 |
| carboxypeptidase E | CPE | b | |
| carboxypeptidase X 1 (M14 family)/metallocarboxypeptidase 1 | CPXM | b | |
| cardiac responsive adriamycin protein | CARP | a | 0.0197 |
| carnitine palmitoyltransferase 1, liver | CPT1A | * | 0.004 |
| carnitine palmitoyltransferase 1, muscle | CPT1B | a | 0.0179 |
| carnitine palmitoyltransferase 2 | CPT2 | a | 0.0005 |
| cartilage oligomeric matrix protein | COMP | a | 0.047 |
| casein kise 1, epsilon | CSNK1E | b | |
| caspase 1 | CASP1 | a | 0.0047 |
| caspase 3, apoptosis related cysteine protease | CASP3 | b | |
| caspase 8 | CASP8 | a | 0.0215 |
| cathepsin D | CTSD | a | 0.0005 |
| cathepsin L | CTSL | a | 0.0157 |
| cathepsin S | CTSS | * | 0.0072 |
| cathepsin Z | CTSZ | a | 0.0285 |
| Cbp/p300-interacting transactivator with Glu/Asp-rich | CITED1 | b | |
| carboxy-termil domain 1 | | | |
| CCCTC-binding factor | CTCF | a | 0.005 |
| CD24a antigen | CD24 | * | 0.0218 |
| CD2-associated protein | CD2AP | (a + b) = * | 0.005 |
| CD38 antigen | CD38 | a | 0.0043 |
| CD48 antigen | CD48 | b | |
| CD52 antigen | CDW52 | (b + b) = b | |
| CD53 antigen | CD53 | * | 0.0096 |
| CD59a antigen | CD59 | a | 0.0013 |
| CD68 antigen | CD68 | * | 0.0005 |
| CD72 antigen | CD72 | * | 0.0018 |
| CDC16 (cell division cycle 16 homolog (S. cerevisiae) | CDC16 | a | 0.0279 |
| CDC28 protein kise 1 | CKS1B | a | 0.0484 |
| CDK2 (cyclin-dependent kise 2)-asscoaited protein 1 | CDK2AP1 | a | 0.0006 |
| CEA-related cell adhesion molecule 1 | CEACAM1 | * | 0.0135 |
| CEA-related cell adhesion molecule 2 | Ceacam2 | * | 0.0015 |
| cell death-inducing D fragmentation factor, alpha subunit-like | CIDEB | a | 0.0031 |
| effector B | | | |
| cell division cycle 2 homolog A (S. pombe) | CDC2 | a | 0.0075 |
| cell division cycle 25 homolog A (S. cerevisiae) | CDC25A | a | 0.0472 |
| cell division cycle 42 homolog (S. cerevisiae) | CDC42 | * | 0.0052 |
| cellular nucleic acid binding protein | ZNF9 | a | 0.0012 |
| centrin 2 | CETN2 | a | 0.0091 |
| centrin 3 | CETN3 | b | |
| ceroid-lipofuscinosis, neurol 2 | CLN2 | a | 0.0041 |
| chaperonin subunit 3 (gamma) | CCT3 | a | 0.001 |
| chemokine (C-C) receptor 2 | CCR2 | * | 0.0215 |
| chemokine (C-C) receptor 5 | CCR5 | a | 0.0046 |
| chemokine orphan receptor 1 | RDC1 | b | |
| chitise 3-like 3 | CHIA | a | 0.03 |
| chloride channel calcium activated 1 | CLCA1 | b | |
| chloride channel, nucleotide-sensitive, 1A | CLNS1A | b | |
| chloride intracellular channel 1 | CLIC1 | * | 0.0005 |
| chloride intracellular channel 4 (mitochondrial) | CLIC4 | * | 0.0186 |
| cholinergic receptor, nicotinic, beta polypeptide 1 (muscle) | CHRNB1 | b | |
| citrate lyase beta like | CLYBL | a | 0.0021 |
| clathrin, light polypeptide (Lca) | CLTA | a | 0.0029 |
| claudin 1 | CLDN1 | * | 0.0005 |
| claudin 4 | CLDN4 | * | 0.0012 |
| claudin 7 | CLDN7 | * | 0.0005 |
| cleavage and polyadenylation specific factor 5, 25 kD subunit | CPSF5 | b | |
| clusterin | CLU | a | 0.0005 |
| coagulation factor II (thrombin) receptor-like 1 | F2RL1 | * | 0.0005 |
| coagulation factor III | F3 | * | 0.0005 |
| coagulation factor XIII, beta subunit | F13B | * | 0.0005 |
| cofilin 1, non-muscle | CFL1 | a | 0.0005 |
| cold shock domain protein A | CSDA | * | 0.0005 |
| colony stimulating factor 1 (macrophage) | CSF1 | a | 0.0011 |
| complement component 1, q subcomponent, alpha polypeptide | C1QA | * | 0.0096 |
| complement component 1, q subcomponent, beta polypeptide | C1QB | b | |
| complement component 1, q subcomponent, c polypeptide | C1QG | b | |
| complement component 3 | C3 | * | 0.0013 |
| complement component factor i | IF | a | 0.004 |
| complement factor H related protein 3A4/5G4 | HF1 | (b + b) = b | |
| connective tissue growth factor | CTGF | b | |
| constitutive photomorphogenic protein 1 (Arabidopsis) | COP1 | b | |
| coproporphyrinogen oxidase | CPO | b | |
| cordon-bleu; ESTs, Moderately similar to T00381 KIAA0633 | COBL | a | 0.0185 |
| protein (H. sapiens) [symbol] - | | | |
| core promoter element binding protein | COPEB | (* + *) = * | 0.0052; |
| | | | 0.0009 |
| cornichon homolog (Drosophila) | CNIH | a | 0.03 |
| coronin, actin binding protein 1B | CORO1B | * | 0.0086 |
| craniofacial development protein 1 | CFDP1 | * | 0.0005 |
| creatine kise, brain | CKB | a | 0.0099 |
| cryptochrome 2 (photolyase-like) | CRY2 | a | 0.0339 |
| crystallin, alpha B | CRYAB | a | 0.0183 |
| crystallin, lamda 1 | CRYL1 | * | 0.0075 |
| crystallin, mu | CRYM | * | 0.0008 |
| cyclin E1 | CCNE1 | a | 0.0146 |
| cyclin-dependent kise 4 | CDK4 | a | 0.0006 |
| cyclin-dependent kise inhibitor 1A (P21) | CDKN1A | a | 0.0005 |
| cystatin B | CSTB | * | 0.0005 |
| cystatin C | CST3 | b | |
| cysteine rich protein 61 | CYR61 | * | 0.0014 |
| cytidine 5′-triphosphate synthase | CTPS | * | 0.0006 |
| cytidine 5′-triphosphate synthase 2 | CTPS2 | b | |
| cytochrome c oxidase, subunit VIc | COX6C | a | 0.0052 |
| cytochrome c oxidase, subunit VIIa 1 | COX7A1 | a | 0.0099 |
| cytochrome c oxidase, subunit VIIa 3 | COX7A3 | a | 0.0497 |
| cytochrome c oxidase, subunit VIIIa | COX8 | b | |
| cytochrome P450, 2a4 | CYP2A13 | (* + *) = * | 0.0008; |
| | | | 0.0186 |
| cytochrome P450, 2d9 | CYP2D6 | (a + b) = * | 0.0005 |
| cytochrome P450, 2e1, ethanol inducible | CYP2E1 | a | 0.0082 |
| cytochrome P450, 2j5 | CYP2J2 | * | 0.005 |
| cytochrome P450, family 4, subfamily v, polypeptide 3/ | Cyp4v3 | b | |
| expressed sequence AW111961 | | | |
| cytochrome P450, subfamily IV B, polypeptide 1 | CYP4B1 | b | |
| cytokine inducible SH2-containing protein 3 | SOCS3 | * | 0.0005 |
| D methyltransferase (cytosine-5) 1 | DNMT1 | a | 0.0015 |
| D methyltransferase 3B | DNMT3B | a | 0.0009 |
| D primase, p49 subunit | PRIM1 | a | 0.0009 |
| D segment, Chr 12, ERATO Doi 604, expressed | TSSC1 | b | |
| D segment, Chr 17, ERATO Doi 441, expressed | D17Ertd441e | * | 0.0072 |
| D segment, Chr 17, human D6S56E 2 | LSM2 | a | 0.0045 |
| D segment, Chr 18, Wayne State University 181, expressed | ALDH7A1 | * | 0.0135 |
| D segment, Chr 8, Brigham & Women's Genetics 1320 | D8Bwg1320e | a | 0.0086 |
| expressed | | | |
| damage specific D binding protein 1 (127 kDa) | DDB1 | a | 0.0014 |
| D-amino acid oxidase | DAO | b | |
| D-dopachrome tautomerase | DDT | a | 0.0008 |
| DEAD/H (Asp-Glu-Ala-Asp/His) box polypeptide 50/ | DDX50 | b | |
| nucleolar protein GU2 | | | |
| decorin | DCN | b | |
| deiodise, iodothyronine, type I | DIO1 | * | 0.0005 |
| deltex 1 homolog (Drosophila) | DTX1 | a | 0.0086 |
| deoxyribonuclease I | DNASE1 | * | 0.0005 |
| diaphorase 1 (DH) | DIA1 | * | 0.0023 |
| dihydropyrimidise | DYPS | * | 0.0021 |
| dihydropyrimidise-like 3 | DPYSL3 | a | 0.0218 |
| dimethylarginine dimethylaminohydrolase 2 | DDAH2 | b | |
| dipeptidase 1 (rel) | DPEP1 | * | 0.0006 |
| DJ (Hsp40) homolog, subfamily A, member 1 | DNAJA1 | a | 0.0005 |
| DJ (Hsp40) homolog, subfamily B, member 12 | Djb12 | a | 0.0035 |
| DJ (Hsp40) homolog, subfamily C, member 5 | DNAJC5 | b | |
| dolichyl-di-phosphooligosaccharide-protein glycotransferase | DDOST | a | 0.0013 |
| dopa decarboxylase | DDC | a | 0.0047 |
| double cortin and calcium/calmodulin-dependent protein kise- | DCAMKL1 | a | 0.0042 |
| like 1 | | | |
| downstream of tyrosine kise 1 | DOK1 | b | |
| DPH oxidase 4 | NOX4 | b | |
| E26 avian leukemia oncogene 2, 3′ domain | ETS2 | a | 0.0012 |
| E74-like factor 3 | ELF3 | * | 0.0312 |
| E74-like factor 4 (ets domain transcription factor) | ELF4 | * | 0.0023 |
| early development regulator 2 (homolog of polyhomeotic 2) | EDR2 | b | |
| ectonucleoside triphosphate diphosphohydrolase 5 | ENTPD5 | a | 0.0313 |
| ectonucleotide pyrophosphatase/phosphodiesterase 2 | ENPP2 | * | 0.0005 |
| EGF-like module containing, mucin-like, hormone receptor- | EMR1 | b | |
| like sequence 1 | | | |
| EGL nine homolog 1 (C. elegans) | EGLN1 | a | 0.0008 |
| elafin-like protein I | SWAM1 | a | 0.0005 |
| elastase 1, pancreatic | ELA1 | a | 0.0005 |
| elongation of very long chain fatty acids (FEN1/Elo2, | ELOVL1 | * | 0.0012 |
| SUR4/Elo3, yeast)-like 1 | | | |
| endonuclease G | ENDOG | a | 0.0014 |
| endoplasmic reticulum protein 29 | C12orf8 | b | |
| endothelin 1 | EDN1 | * | 0.0057 |
| enhancer of zeste homolog 2 (Drosophila) | EZH2 | a | 0.0018 |
| enoyl Coenzyme A hydratase, short chain, 1, mitochondrial | ECHS1 | a | 0.0005 |
| epidermal growth factor | EGF | * | 0.0005 |
| epidermal growth factor-containing fibulin-like extracellular | EFEMP1 | b | |
| matrix protein 1 | | | |
| epidermal growth factor-containing fibulin-like extracellular | EFEMP2 | * | 0.0006 |
| matrix protein 2 | | | |
| epithelial membrane protein 3 | EMP3 | * | 0.0009 |
| erythrocyte protein band 4.1/Mus musculus adult male tongue | EPB41 | b | |
| cD, RIKEN full-length enriched library, | | | |
| clone:2310065B16:erythrocyte protein band 4.1, full insert | | | |
| sequence | | | |
| erythrocyte protein band 4.1-like 1 | EPB41L1 | a | 0.0009 |
| erythroid differentiation regulator | edr | a | 0.0424 |
| EST AI181838 | MGC2555 | a | 0.0005 |
| estrogen related receptor, alpha | ESRRA | a | 0.0023 |
| ESTs | | * | 0.0041 |
| ESTs | | * | 0.006 |
| ESTs | | a | 0.0022 |
| ESTs | | a | 0.0012 |
| ESTs | | a | 0.0125 |
| ESTs | | a | 0.0014 |
| ESTs | | a | 0.0381 |
| ESTs | Rin3 | a | 0.0012 |
| ESTs | | a | 0.0006 |
| ESTs | | a | 0.0026 |
| ESTs | | a | 0.0006 |
| ESTs | | a | 0.0005 |
| ESTs | | a | 0.0048 |
| ESTs | | a | 0.0015 |
| ESTs | | a | 0.0217 |
| ESTs | | a | 0.03 |
| ESTs | | a | 0.0072 |
| ESTs | | a | 0.018 |
| ESTs | | a | 0.0005 |
| ESTs | | a | 0.0118 |
| ESTs | | a | 0.0067 |
| ESTs | | a | 0.0307 |
| ESTs | | a | 0.0023 |
| ESTs | | a | 0.0018 |
| ESTs | | a | 0.0381 |
| ESTs | | a | 0.0013 |
| ESTs | | a | 0.0268 |
| ESTs | | a | 0.0033 |
| ESTs | | b | |
| ESTs | | b | |
| ESTs | | b | |
| ESTs | | b | |
| ESTs | FLJ22184 | b | |
| ESTs | | b | |
| ESTs | 9130203F04Rik | b | |
| ESTs | | b | |
| ESTs | | b | |
| ESTs | | b | |
| ESTs | 1110069O07Rik | b | |
| ESTs | FLJ23447 | b | |
| ESTs | | b | |
| ESTs | | b | |
| ESTs - pending | PCSK9 | a | 0.0031 |
| ESTs, Highly similar to prefoldin 4 (Homo sapiens) | PFDN4 | a | 0.006 |
| (H. sapiens) | | | |
| ESTs, Highly similar to organic cation transporter-like protein | | a | 0.0015 |
| 2 (M. musculus) | | | |
| ESTs, Highly similar to T00268 hypothetical protein | KIAA0597 | a | 0.0005 |
| KIAA0597 (H. sapiens) | | | |
| ESTs, Moderately similar to SEC7 homolog (Homo sapiens) | | b | |
| (H. sapiens) | | | |
| ESTs, Moderately similar to S12207 hypothetical protein | | * | 0.0005 |
| (M. musculus) | | | |
| ESTs, Moderately similar to T08673 hypothetical protein | KIAA0977 | * | 0.0343 |
| DKFZp564C0222.1 (H. sapiens) | | | |
| ESTs, Moderately similar to T46312 hypothetical protein | | b | |
| DKFZp434J1111.1 (H. sapiens) | | | |
| ESTs, Weakly similar to brain-specific angiogenesis inhibitor | | a | 0.0219 |
| 1-associated protein 2 (Mus musculus) (M. musculus) | | | |
| ESTs, Weakly similar to limb expression 1 homolog (chicken) | | a | 0.0118 |
| (Mus musculus) (M. musculus) | | | |
| ESTs, Weakly similar to simple repeat sequence-containing | | b | |
| transcript (Mus musculus) (M. musculus) | | | |
| ESTs, Weakly similar to 2022314A granule cell marker protein | | b | |
| (M. musculus) | | | |
| ESTs, Weakly similar to ADT1 MOUSE ADP, ATP CARRIER | | a | 0.0018 |
| PROTEIN, HEART/SKELETAL MUSCLE ISOFORM T1 | | | |
| (M. musculus) | | | |
| ESTs, Weakly similar to ADT1 MOUSE ADP, ATP CARRIER | SLC25A16 | a | 0.0133 |
| PROTEIN, HEART/SKELETAL MUSCLE ISOFORM T1 | | | |
| (M. musculus) | | | |
| ESTs, Weakly similar to AF182426 1 arylacetamide | | * | 0.0472 |
| deacetylase (R. norvegicus) | | | |
| ESTs, Weakly similar to B Chain B, Crystal Structure Of | | b | |
| Murine Soluble Epoxide Hydrolase Complexed With Cdu | | | |
| Inhibitor (M. musculus) | | | |
| ESTs, Weakly similar to DRR1 (H. sapiens) | | * | 0.0017 |
| ESTs, Weakly similar to JC7182 +-dependent vitamin C | SLC23A3 | a | 0.0472 |
| (H. sapiens) | | | |
| ESTs, Weakly similar to JE0096 myocilin - mouse | | b | |
| (M. musculus) | | | |
| ESTs, Weakly similar to MAJOR URIRY PROTEIN 4 | | b | |
| PRECURSOR (M. musculus) | | | |
| ESTs, Weakly similar to S26689 hypothetical protein hc1 - | | a | 0.0135 |
| mouse (M. musculus) | | | |
| ESTs, Weakly similar to S65210 hypothetical protein YPL191c - | | a | 0.0049 |
| yeast (Saccharomyces cerevisiae) (S. cerevisiae) | | | |
| ESTs, Weakly similar to T29029 hypothetical protein | 4931439A04Rik | a | 0.0006 |
| F53G12.5 - Caenorhabditis elegans (C. elegans) | | | |
| ESTs, Weakly similar to TS13 MOUSE TESTIS-SPECIFIC | MGC39016 | b | |
| PROTEIN PBS13 (M. musculus) | | | |
| ESTs, Weakly similar to TYROSINE-PROTEIN KISE JAK3 | | * | 0.0147 |
| (M. musculus) | | | |
| ESTs, Weakly similar to TYROSINE-PROTEIN KISE JAK3 | | a | 0.0086 |
| (M. musculus) | | | |
| ESTs, Weakly similar to TYROSINE-PROTEIN KISE JAK3 | C1QR1 | a | 0.0185 |
| (M. musculus) | | | |
| ESTs, Weakly similar to YAE6_YEAST HYPOTHETICAL | | a | 0.0175 |
| 13.4 KD PROTEIN IN ACS1-GCV3 INTERGENIC REGION | | | |
| (S. cerevisiae) | | | |
| ESTs, Weakly similar to YMP2_CAEEL HYPOTHETICAL | 3230401L03Rik | * | 0.0005 |
| 30.3 KD PROTEIN B0361.2 IN CHROMOSOME III | | | |
| (C. elegans) | | | |
| eukaryotic translation initiation factor 2A | eIF2a | b | |
| eukaryotic translation initiation factor 3 | EIF3S10 | a | 0.0016 |
| eukaryotic translation initiation factor 3, subunit 4 (delta, 44 kDa) | EIF3S4 | a | 0.0009 |
| eukaryotic translation initiation factor 4, gamma 2 | EIF4G2 | a | 0.0424 |
| eukaryatic translation initiation factor 4A1 | EIF4A1 | * | 0.0135 |
| eukaryotic translation initiation factor 4A2 | EIF4A2 | a | 0.0014 |
| eukaryotic translation initiation factor 4E binding protein 1 | EIF4EBP1 | * | 0.0078 |
| eukaryotic translation initiation factor 5A | EIF5A | a | 0.0005 |
| E-vasodilator stimulated phosphoprotein | EVL | b | |
| exportin 1, CRM1 homolog (yeast) | XPO1 | a | 0.0008 |
| expressed in non-metastatic cells 2, protein (NM23B) | NME2 | a | 0.0096 |
| (nucleoside diphosphate kise) | | | |
| expressed sequence AA408783 | SPEC2 | b | |
| expressed sequence AA589392 | AA589392 | a | 0.0011 |
| expressed sequence AA672638 | AA672638 | a | 0.0201 |
| expressed sequence AI117581 | AI117581 | a | 0.0424 |
| expressed sequence AI118577 | ZNF14 | (a + b) = * | 0.0005 |
| expressed sequence AI132189 | AI132189 | a | 0.0068 |
| expressed sequence AI132321 | AI132321 | * | 0.0086 |
| expressed sequence AI159688 | AI159688 | * | 0.0006 |
| expressed sequence AI182282 | SLC9A8 | a | 0.0005 |
| expressed sequence AI182284 | AI182284 | * | 0.0012 |
| expressed sequence AI194696 | HFL1 | b | |
| expressed sequence AI265322 | AI265322 | a | 0.0016 |
| expressed sequence AI314027 | GLS | b | |
| expressed sequence AI315037 | AI315037 | a | 0.0117 |
| expressed sequence AI316828 | FLJ20618 | b | |
| expressed sequence AI413331 | AI413331 | b | |
| expressed sequence AI447451 | AI447451 | b | |
| expressed sequence AI448003 | AI448003 | b | |
| expressed sequence AI449309 | AI449309 | b | |
| expressed sequence AI450991 | KIAA0729 | a | 0.0285 |
| expressed sequence AI461788 | AI461788 | a | 0.0026 |
| expressed sequence AI465301 | AI465301 | a | 0.0021 |
| expressed sequence AI480660 | AI480660 | a | 0.0012 |
| expressed sequence AI504062 | AI504062 | * | 0.033 |
| expressed sequence AI507121 | AI507121 | a | 0.0005 |
| expressed sequence AI528491 | AI528491 | a | 0.0208 |
| expressed sequence AI553555 | AI553555 | a | 0.0018 |
| expressed sequence AI558103 | LRRN1 | a | 0.025 |
| expressed sequence AI586180 | AI586180 | * | 0.0231 |
| expressed sequence AI593249 | AI593249 | * | 0.0005 |
| expressed sequence AI593524 | DKFZp586A011.1 | b | |
| expressed sequence AI604920 | KIAA0297 KIAA0329 | b | |
| expressed sequence AI607846 | AIF1 | * | 0.0116 |
| expressed sequence AI646725 | MDS028 | b | |
| expressed sequence AI661919 | AI661919 | b | |
| expressed sequence AI835705 | AI835705 | a | 0.0012 |
| expressed sequence AI836219 | AI836219 | a | 0.0165 |
| expressed sequence AI838057 | AI838057 | a | 0.0013 |
| expressed sequence AI843960 | RBPSUH | b | |
| expressed sequence AI844685 | MGC15429 | a | 0.0014 |
| expressed sequence AI844876 | AI844876 | b | |
| expressed sequence AI848669 | AI848669 | a | 0.0497 |
| expressed sequence AI852479 | CDKL3 | a | 0.0005 |
| expressed sequence AI875199 | AI875199 | a | 0.0041 |
| expressed sequence AI875557 | AI875557 | a | 0.0009 |
| expressed sequence AI957255 | KIAA0564 | a | 0.0012 |
| expressed sequence AI987692 | AI987692 | b | |
| expressed sequence AL022757 | 5730453I16Rik | a | 0.0005 |
| expressed sequence AU015645 | AU015645 | * | 0.0006 |
| expressed sequence AU018056 | AU018056 | a | 0.0068 |
| expressed sequence AU019833 | C1orf24 | b | |
| expressed sequence AU042434 | AU042434 | b | |
| expressed sequence AV046379 | AV046379 | * | 0.0012 |
| expressed sequence AW045860 | AW045860 | b | |
| expressed sequence AW047581 | AW047581 | b | |
| expressed sequence AW124722 | AW124722 | a | 0.0316 |
| expressed sequence AW261723 | SLC17A3 | * | 0.0025 |
| expressed sequence AW413625 | FLJ22794 | a | 0.0497 |
| expressed sequence AW488255 | EFNB1 | a | 0.0477 |
| expressed sequence AW493404 | AW493404 | b | |
| expressed sequence AW541137 | NUP107 | b | |
| expressed sequence AW552393 | AW552393 | a | 0.0239 |
| expressed sequence AW743884 | AW743884 | b | |
| expressed sequence BB120430 | BB120430 | a | 0.0099 |
| expressed sequence C79732 | C79732 | a | 0.0005 |
| expressed sequence C80913 | C80913 | b | |
| expressed sequence C81457 | FLJ21022 | b | |
| expressed sequence C85317 | C85317 | b | |
| expressed sequence C85457 | C85457 | a | 0.0483 |
| expressed sequence C86169 | C86169 | a | 0.0046 |
| expressed sequence C86302 | C86302 | a | 0.0013 |
| expressed sequence C87222 | C87222 | * | 0.0012 |
| expressed sequence R75232 | R75232 | a | 0.001 |
| Fas apoptotic inhibitory molecule | FAIM | b | |
| fatty acid synthase | FASN | a | 0.0023 |
| f-box only protein 3 | FBXO3 | a | 0.0119 |
| Fc receptor, IgE, high affinity I, gamma polypeptide | FCER1G | * | 0.0023 |
| Fc receptor, IgG, low affinity III | FCGR3A | * | 0.0025 |
| feline sarcoma oncogene | FES | a | 0.01 |
| fibrillarin | FBL | a | 0.0068 |
| fibrillin 1 | FBN1 | * | 0.0009 |
| fibulin 5 | FBLN5 | a | 0.002 |
| FK506 binding protein 10 (65 kDa) | FKBP10 | a | 0.0005 |
| FK506 binding protein 12-rapamycin associated protein 1 | FRAP1 | * | 0.0022 |
| FK506 binding protein 1a (12 kDa) | FKBP1A | a | 0.0005 |
| FK506 binding protein 5 (51 kDa) | FKBP5 | b | |
| FK506 binding protein 9 | FKBP9 | a | 0.0347 |
| flap structure specific endonuclease 1 | FEN1 | a | 0.0398 |
| flavin containing monooxygese 1 | FMO1 | a | 0.0159 |
| flotillin 1 | FLOT1 | a | 0.0005 |
| flotillin 2 | FLOT2 | a | 0.0103 |
| folate receptor 1 (adult) | FOLR1 | * | 0.0008 |
| forkhead box M [symbol] | FOXM1 | a | 0.0023 |
| four and a half LIM domains 1 | FHL1 | b | |
| fragile histidine triad gene | FHIT | a | 0.0026 |
| fumarylacetoacetate hydrolase | FAH | * | 0.0008 |
| FXYD domain-containing ion transport regulator 2 | FXYD2 | b | |
| FXYD domain-containing ion transport regulator 5 | FXYD5 | * | 0.0005 |
| G protein-coupled receptor kise 7 | MKNK2 | a | 0.001 |
| G1 to phase transition 1 | GSPT1 | a | 0.0331 |
| gamma-glutamyl hydrolase | GGH | b | |
| gamma-glutamyl transpeptidase | GGT1 | * | 0.0047 |
| ganglioside-induced differentiation-associated-protein 3 | MRPS33 | b | |
| gap junction membrane channel protein beta 2 | GJB2 | b | |
| glucose regulated protein, 58 kDa | GRP58 | a | 0.006 |
| glucose-6-phosphatase, catalytic | G6PC | * | 0.0046 |
| glucose-6-phosphatase, transport protein 1 | G6PT1 | a | 0.0005 |
| glutamine synthetase | GLUL | (* + *) = * | 0.0179 |
| glutaryl-Coenzyme A dehydrogese | GCDH | * | 0.0034 |
| glutathione peroxidase 1 | GPX1 | a | 0.0177 |
| glutathione S-transferase, alpha 2 (Yc2) | GSTA2 | b | |
| glutathione S-transferase, alpha 4 | GSTA4 | b | |
| glutathione S-transferase, mu 6 | GSTM1 | a | 0.0096 |
| glutathione S-transferase, pi 1 | GSTP1 | a | 0.0124 |
| glutathione S-transferase, theta 2 | GSTT2 | a | 0.0013 |
| glutathione transferase zeta 1 (maleylacetoacetate isomerase) | GSTZ1 | a | 0.0009 |
| glycerol kise | GK | * | 0.0287 |
| glycerol phosphate dehydrogese 1, mitochondrial | GPD2 | b | |
| glycerol-3-phosphate acyltransferase, mitochondrial | GPAT | * | 0.0005 |
| glycine amidinotransferase (L-arginine:glycine | GATM | * | 0.0005 |
| amidinotransferase) | | | |
| glycine N-methyltransferase | GNMT | a | 0.0422 |
| glycoprotein 49 A | Gp49a | * | 0.0006 |
| glycoprotein 49 B | Gp49b | * | 0.0005 |
| glypican 3 | GPC3 | b | |
| golgi autoantigen, golgin subfamily a, 4 | GOLGA4 | a | 0.0009 |
| golgi reassembly stacking protein 2 | GORASP2 | * | 0.005 |
| GPI-anchored membrane protein 1 | M11S1 | a | 0.0115 |
| granulin | GRN | a | 0.0227 |
| G-rich RNA sequence binding factor 1 (D5Wsu31e) D | GRSF1 | b | |
| segment, Chr 5, Wayne State University 31, expressed | | | |
| group specific component | GC | a | 0.0466 |
| growth arrest and D-damage-inducible 45 alpha | GADD45A | * | 0.0008 |
| growth arrest and D-damage-inducible 45 gamma | GADD45G | b | |
| growth arrest specific 2 | GAS2 | * | 0.0008 |
| growth differentiation factor 15 | PLAB | * | 0.0047 |
| growth differentiation factor 8 | GDF8 | b | |
| growth factor receptor bound protein 7 | GRB7 | a | 0.0013 |
| guanine nucleotide binding protein (G protein), gamma 2 | GNG2 | b | |
| subunit | | | |
| guanine nucleotide binding protein (G protein), gamma 5 | GNG5 | * | 0.0005 |
| subunit | | | |
| guanine nucleotide binding protein, alpha inhibiting 2 | GNAI2 | * | 0.0067 |
| guanine nucleotide binding protein, beta 2, related sequence 1 | GNB2L1 | * | 0.0005 |
| guanosine diphosphate (GDP) dissociation inhibitor 3 | GDI-3 | a | 0.0312 |
| guanosine monophosphate reductase | GMPR | * | 0.0086 |
| guanylate nucleotide binding protein 2 | GBP2 | b | |
| H2A histone family, member Z | H2AFZ | * | 0.0068 |
| H2B histone family, member S | H2BFS | a | 0.0005 |
| Harvey rat sarcoma oncogene, subgroup R | RRAS | a | 0.0006 |
| heat shock 70 kDa protein 4 | HSPA4 | (a + a) = a | 0.0047; |
| | | | 0.001 |
| heat shock protein 1 (chaperonin)/heat shock protein, 60 kDa | HSPD1 | b | |
| heat shock protein, 105 kDa | HSPH1 | b | |
| heat shock protein, 86 kDa 1 | HSPCA | a | 0.0013 |
| heat-responsive protein 12 | UK114 | a | 0.0005 |
| hematological and neurological expressed sequence 1 | HN1 | a | 0.0008 |
| heme oxygese (decycling) 1 | HMOX1 | a | 0.0393 |
| hemochromatosis | HFE | b | |
| hemopoietic cell phosphatase | PTPN6 | * | 0.0005 |
| heparan sulfate 2-O-sulfotransferase 1 | HS2ST1 | a | 0.0047 |
| heparin binding epidermal growth factor-like growth factor | DTR | a | 0.019 |
| hepatic nuclear factor 4 | HNF4A | b | |
| hepatoma-derived growth factor | HDGF | a | 0.0377 |
| hepsin | HPN | * | 0.0018 |
| heterogeneous nuclear ribonucleoprotein A1 | HNRPA1 | * | 0.0005 |
| hexokise 1 | HK1 | a | 0.0381 |
| high mobility group AT-hook 1 | HMGA1 | a | 0.0005 |
| high mobility group box 3 | HMGB3 | * | 0.0012 |
| high mobility group nucleosomal binding domain 2 | HMGN2 | * | 0.0014 |
| histidyl tR synthetase | HARS | a | 0.0146 |
| histocompatibility 2, class II antigen A, alpha | HLA-DQA1 | b | |
| histocompatibility 2, class II antigen E beta | H2-Eb1 | b | |
| histocompatibility 2, class II, locus DMa | HLA-DMA | b | |
| Histocompatibiity 2, D region locus 1 | H2-D1 | * | 0.0012 |
| histocompatibility 2, Q region locus 7 | H2-Q7 | b | |
| histone 2, H2aa1/(Hist2) histone gene complex 2 | HIST2H2AA | b | |
| histone deacetylase 1 | HDAC1 | b | |
| homeo box B7 | HOXB7 | a | 0.025 |
| homocysteine-inducible, endoplasmic reticulum stress- | HERPUD1 | * | 0.0092 |
| inducible, ubiquitin-like domain member 1 | | | |
| Hoxc8 | MCM5 | a | 0.0005 |
| Hprt | HPRT1 | a | 0.001 |
| hyaluron mediated motility receptor (RHAMM) | HMMR | a | 0.0171 |
| hyaluronic acid binding protein 2 | HABP2 | b | |
| hydroxysteroid 17-beta dehydrogese 7 | HSD17B7 | b | |
| hydroxysteroid dehydrogese-1, delta<5>-3-beta | HSD3B2 | a | 0.0119 |
| hydroxysteroid dehydrogese-3, delta<5>-3-beta | Hsd3b3 | a | 0.0018 |
| hypothetical protein, I54 | X61497 | * | 0.0005 |
| hypothetical protein, MGC: 6957 | MGC6957 | b | |
| hypothetical protein, MNCb-5210 | COBRA1 | b | |
| Ia-associated invariant chain | CD74 | b | |
| immunoglobulin superfamily, member 8 | IGSF8 | a | 0.0338 |
| importin 11 (RIKEN cD 2510001A17 gene) | IPO11 | a | 0.0056 |
| inhibin beta-B | INHBB | a | 0.0005 |
| inhibitor of D binding 2 | ID2 | b | |
| inosine 5′-phosphate dehydrogese 2 | IMPDH2 | a | 0.0005 |
| inositol polyphosphate-5-phosphatase, 75 kDa | INPP5B | * | 0.0005 |
| insulin-like growth factor binding protein 1 | IGFBP1 | a | 0.0005 |
| insulin-like growth factor binding protein 3 | IGFBP3 | a | 0.0005 |
| insulin-like growth factor binding protein 4 | IGFBP4 | a | 0.0005 |
| insulin-like growth factor binding protein, acid labile subunit | IGFALS | a | 0.0013 |
| integrin alpha 6 | ITGA6 | b | |
| integrin alpha M | ITGAM | a | 0.0224 |
| integrin beta 1 (fibronectin receptor beta) | ITGB1 | b | |
| integrin-associated protein | CD47 | b | |
| intercellular adhesion molecule | ICAM1 | * | 0.0006 |
| interferon activated gene 204 | Ifi204 | (b + b) = b | |
| interferon gamma receptor | IFNGR1 | b | |
| interferon inducible protein 1 | Ifi1 | a | 0.0005 |
| interferon-induced protein with tetratricopeptide repeats 3 | IFIT3 | a | 0.0006 |
| intergral membrane protein 1 | ITM1 | a | 0.0047 |
| interleukin 1 beta | IL1B | a | 0.0023 |
| interleukin 1 receptor, type I | IL1R1 | a | 0.0021 |
| interleukin 11 receptor, alpha chain 1 | IL11RA | a | 0.0043 |
| isocitrate dehydrogese 2 (DP+), mitochondrial | IDH2 | * | 0.0023 |
| isovaleryl coenzyme A dehydrogese | IVD | (* + a) = * | 0.0009; |
| | | | 0.0005 |
| J domain protein 1 | JDP1 | * | 0.0021 |
| junction plakoglobin | JUP | a | 0.0008 |
| kallikrein 26 | Klk26 | * | 0.0005 |
| kallikrein 6 | Klk1/6 | * | 0.0417 |
| karyopherin (importin) alpha 2 | KPNA2 | a | 0.0005 |
| karyopherin (importin) beta 3 | KPNB3 | a | 0.0068 |
| keratin complex 1, acidic, gene 19 | KRT19 | b | |
| keratin complex 2, basic, gene 8 | KRT8 | * | 0.0005 |
| ketohexokise | KHK | * | 0.0005 |
| kidney-derived aspartic protease-like protein | NAP1 | * | 0.005 |
| kinectin 1 | KTN1 | b | |
| kinesin family member 1B (expressed sequence AI448212) | KIF1B | a | 0.0159 |
| kinesin family member 21A | KIF21A | a | 0.0031 |
| kise insert domain protein receptor | KDR | a | 0.0026 |
| klotho | KL | * | 0.0005 |
| Kruppel-like factor 1 (erythroid) | KLF1 | a | 0.0006 |
| Kruppel-like factor 15 | KLF15 | * | 0.0005 |
| Kruppel-like factor 5 | KLF5 | a | 0.0352 |
| Kruppel-like factor 9 | BTEB1 | * | 0.0005 |
| kynurenise (L-kynurenine hydrolase) | KYNU | a | 0.0166 |
| L-3-hydroxyacyl-Coenzyme A dehydrogese, short chain | HADHSC | * | 0.0176 |
| lactate dehydrogese 1, A chain | LDHA | a | 0.0096 |
| laminin B1 subunit 1 | LDAMB1 | a | 0.0321 |
| laminin receptor 1 (67 kD, ribosomal protein SA) | LAMR1 | * | 0.0139 |
| laminin, alpha 2 | LAMA2 | b | |
| latexin | LXN | a | 0.0201 |
| lectin, galactose binding, soluble 3 | LGALS3 | * | 0.0005 |
| lectin, galactose binding, soluble 4 | LGALS4 | a | 0.0295 |
| lectin, galactose binding, soluble 9 | LGALS9 | a | 0.0096 |
| leucine zipper-EF-hand containing transmembrane protein 1 | LETM1 | * | 0.0006 |
| leucocyte specific transcript 1 | LY117 | b | |
| leukemia-associated gene | STMN1 | a | 0.0123 |
| leukotriene C4 synthase | LTC4S | a | 0.0058 |
| LIM and SH3 protein 1 | LASP1 | b | |
| lipoprotein lipase | LPL | * | 0.0008 |
| liver-specific bHLH-Zip transcription factor | Lisch7 | b | |
| low density lipoprotein receptor-related protein 2 | LRP2 | a | 0.0155 |
| low density lipoprotein receptor-related protein 6 | LRP6 | a | 0.0201 |
| LPS-induced TNF-alpha factor | LITAF | * | 0.0005 |
| lymphocyte antigen 6 complex, locus A | | a | 0.0005 |
| lymphocyte antigen 6 complex, locus E | LY6E | * | 0.0005 |
| lymphocyte specific 1 | LSP1 | * | 0.0126 |
| lyric (D8Bwg1112e) D segment, Chr 8, Brigham & Women's | LYRIC | b | |
| Genetics 1112 expressed | | | |
| lysosomal-associated protein transmembrane 4A | LAPTM4A | b | |
| lysosomal-associated protein transmembrane 4B | LAPTM4B | b | |
| lysosomal-associated protein transmembrane 5 | LAPTM5 | b | |
| lysozyme | LYZ | b | |
| lysyl oxidase-like | LOXL1 | a | 0.0008 |
| M. musculus mR for protein expressed at high levels in testis | Tex2 | b | |
| macrophage expressed gene 1 | MPEG1 | * | 0.025 |
| macrophage migration inhibitory factor | MIF | b | |
| macrophage scavenger receptor 2 | Msr2 | b | |
| MAD homolog 5 (Drosophila)/expressed sequence AI451355 | MADH5 | b | |
| mago-shi homolog, proliferation-associated (Drosophila) | MAGOH | a | 0.0068 |
| major vault protein | MVP | a | 0.0013 |
| malate dehydrogese, soluble | MDH1 | * | 0.0011 |
| malic enzyme, supertant | ME1 | * | 0.0005 |
| malonyl-CoA decarboxylase | MLYCD | * | 0.0009 |
| mammary tumor integration site 6 | EIF3S6 | * | 0.0102 |
| mannose receptor, C type 1 | MRC1 | b | |
| mannose-6-phosphate receptor, cation dependent | M6PR | b | |
| MARCKS-like protein | MLP | b | |
| matrix gamma-carboxyglutamate (gla) protein | MGP | * | 0.0424 |
| matrix metalloproteise 14 (membrane-inserted) | MMP14 | b | |
| matrix metalloproteise 2 | MMP2 | b | |
| matrix metalloproteise 23 | MMP23A | b | |
| matrix metalloproteise 7 | MMP7 | b | |
| max binding protein | MNT | b | |
| melanoma antigen, family D, 2 | MAGED2 | * | 0.0201 |
| meprin 1 alpha | MEP1A | * | 0.0155 |
| metallothionein 1 | MT1A | * | 0.0047 |
| metallothionein 2 | MT2A | a | 0.0023 |
| metastasis associated 1-like 1 | MTA1L1 | b | |
| methionine aminopeptidase 2 | METAP2 | a | 0.0123 |
| methyl CpG binding protein 2 | MECP2 | b | |
| methylenetetrahydrofolate dehydrogese (DP+ dependent), | MTHFD1 | * | 0.0054 |
| methenyltetrahydrofolate cyclohydrolase, | | | |
| formyltetrahydrofolate synthase | | | |
| methylmalonyl-Coenzyme A mutase | MUT | * | 0.0012 |
| microfibrillar associated protein 5 | MGP2 | b | |
| microtubule associated testis specific serine/threonine protein | MAST205 | a | 0.0216 |
| kise | | | |
| microtubule-associated protein tau | MAPT | a | 0.0006 |
| microtubule-associated protein, RP/EB family, member 1 | MAPRE1 | a | 0.0119 |
| mini chromosome maintence deficient (S. cerevisiae) | MCM3 | a | 0.0005 |
| mini chromosome maintence deficient 2 (S. cerevisiae) | MCM2 | a | 0.0015 |
| mini chromosome maintence deficient 4 homolog (S. cerevisiae) | MCM4 | a | 0.0005 |
| mini chromosome maintence deficient 7 (S. cerevisiae) | MCM7 | a | 0.039 |
| mitochondrial ribosomal protein L39 | MRPL39 | a | 0.0125 |
| mitochondrial ribosomal protein L50; (D4Wsu125e) D | MRPL50 | a | 0.0343 |
| segment, Chr 4, Wayne State University 125, expressed | | | |
| Mitogen activated protein kinase 1; | MAPK1 | a | 0.0439 |
| RIKEN cD 9030612K14 gene | | | |
| mitogen activated protein kise 13 | MAPK13 | a | 0.0054 |
| mitogen activated protein kise kise kise 1 | MAP3K1 | a | 0.0012 |
| mitogen-activated protein kise 7 | MAPK7 | a | 0.025 |
| mitsugumin 29 | Mg29 | a | 0.0389 |
| MORF-related gene X | MORF4L2 | a | 0.0012 |
| Muf1 protein (D630045E04Rik) Mus musculus, clone | MUF1 | b | |
| IMAGE: 3491421, mR, partial cds | | | |
| Mus musculus adult male kidney cD, RIKEN full-length | | a | 0.0005 |
| enriched library, clone:0610012C11:homogentisate 1,2- | | | |
| dioxygese, full insert sequence | | | |
| Mus musculus adult male liver cD, RIKEN full-length enriched | CSAD | a | 0.0005 |
| library, clone:1300015E02:deoxyribonuclease II alpha, full | | | |
| insert sequence | | | |
| Mus musculus chemokine receptor CCX CKR mR, complete | CCRL1 | * | 0.0005 |
| cds, altertively spliced | | | |
| Mus musculus evectin-2 (Evt2) mR, complete cds | PLEKHB2 | a | 0.0005 |
| Mus musculus LDLR dan mR, complete cds | | a | 0.01 |
| Mus musculus mR for 67 kDa polymerase-associated factor | EIF3S6IP | a | 0.007 |
| PAF67 (paf67 gene) | | | |
| Mus musculus mR for alpha-albumin protein | AFM | a | 0.0005 |
| Mus musculus, basic transcription factor 3, clone MGC: 6799 | LOC218490 | a | 0.0005 |
| IMAGE:2648048, mR, complete cds | | | |
| Mus musculus, clone IMAGE: 3155544, mR, partial cds | LOC224650 | a | 0.0467 |
| Mus musculus, clone IMAGE: 3494258, mR, partial cds | | * | 0.0009 |
| Mus musculus, clone IMAGE: 3586777, mR, partial cds | DLAT | * | 0.0019 |
| Mus musculus, clone IMAGE: 3589087, mR, partial cds | | a | 0.0047 |
| Mus musculus, clone IMAGE: 3967158, mR, partial cds | C13orf11 | a | 0.0424 |
| Mus musculus, clone IMAGE: 3994696, mR, partial cds | YUP8H12R.13 | b | |
| Mus musculus, clone IMAGE: 4456744, mR, partial cds | G630055P03Ri | a | 0.0151 |
| Mus musculus, clone IMAGE: 4486265, mR, partial cds | | a | 0.0021 |
| Mus musculus, clone IMAGE: 4952483, mR, partial cds | TOR2A | b | |
| Mus musculus, clone IMAGE: 4974221, mR, partial cds | APEH | a | 0.0085 |
| Mus musculus, clone MGC: 12039 IMAGE: 3603661, mR, | Itpr5 | a | 0.0119 |
| complete cds | | | |
| Mus musculus, clone MGC: 12159 IMAGE: 3711169, mR, | D530037I19Rik | b | |
| complete cds | | | |
| Mus musculus, clone MGC: 18871 IMAGE: 4234793, mR, | GLYAT | (b + b) = b | |
| complete cds | | | |
| Mus musculus, clone MGC: 18985 IMAGE: 4011674, mR, | FLJ20303 | a | 0.0068 |
| complete cds | | | |
| Mus musculus, clone MGC: 19042 IMAGE: 4188988, mR, | OGDH | a | 0.0008 |
| complete cds | | | |
| Mus musculus, clone MGC: 19361 IMAGE: 4242170, mR, | | a | 0.0424 |
| complete cds | | | |
| Mus musculus, clone MGC: 29021 IMAGE: 3495957, mR, | TAO1 | a | 0.0042 |
| complete cds | | | |
| Mus musculus, clone MGC: 36388 IMAGE: 5098924, mR, | MCSC | * | 0.0233 |
| complete cds | | | |
| Mus musculus, clone MGC: 36554 IMAGE: 4954874, mR, | D14Ertd226e | b | |
| complete cds | | | |
| Mus musculus, clone MGC: 36997 IMAGE: 4948448, mR, | MGC36997 | a | 0.0472 |
| complete cds | | | |
| Mus musculus, clone MGC: 37818 IMAGE: 5098655, mR, | MGC37818 | * | 0.004 |
| complete cds | | | |
| Mus musculus, clone MGC: 38363 IMAGE: 5344986, mR, | TM4SF3 | b | |
| complete cds | | | |
| Mus musculus, clone MGC: 38798 IMAGE: 5359803, mR, | MGC38798 | a | 0.0013 |
| complete cds | | | |
| Mus musculus, clone MGC: 6377 IMAGE: 3499365, mR, | ME2 | a | 0.024 |
| complete cds | | | |
| Mus musculus, clone MGC: 6545 IMAGE: 2655444, mR, | MAT2A | a | 0.0008 |
| complete cds | | | |
| Mus musculus, clone MGC:7898 IMAGE: 3582717, mR, | | * | 0.0012 |
| complete cds | | | |
| Mus musculus, hypothetical protein MGC11287 similar to | RPS6KL1 | a | 0.0343 |
| ribosomal protein S6 kise,, clone MGC: 28043 | | | |
| IMAGE: 3672127, mR, complete cds | | | |
| Mus musculus, Similar to 60S ribosomal protein L30 isolog, | | a | 0.0041 |
| clone MGC: 6735 IMAGE: 3590401, mR, complete cds | | | |
| Mus musculus, Similar to angiopoietin-like factor, clone | | b | |
| MGC: 32448 IMAGE: 5043159, mR, complete cds | | | |
| Mus musculus, Similar to CGI-147 protein, clone MGC: 25743 | | * | 0.025 |
| IMAGE: 3990061, mR, complete cds | | | |
| Mus musculus, Similar to chromosome 20 open reading frame | FLJ10883 | * | 0.0159 |
| 36, clone IMAGE: 5356821, mR, partial cds | | | |
| Mus musculus, Similar to cortactin isoform B, clone | EMS1 | a | 0.0018 |
| MGC: 18474 IMAGE: 3981559, mR, complete cds | | | |
| Mus musculus, Similar to dendritic cell protein, clone | GA17 | * | 0.019 |
| MGC: 11741 IMAGE: 3969335, mR, complete cds | | | |
| Mus musculus, Similar to DKFZP586B0621 protein, clone | C1QTNF5 | b | |
| MGC: 38635 IMAGE: 5355789, mR, complete cds | | | |
| Mus musculus, similar to heterogeneous nuclear | MGC37309 | * | 0.0005 |
| ribonucleoprotein A3 (H. sapiens), clone MGC: 37309 | | | |
| IMAGE: 4975085, mR, complete cds | | | |
| Mus musculus, Similar to hypothetical protein | DKFZp566A1524 | a | 0.013 |
| DKFZp566A1524, clone MGC: 18989 IMAGE: 4012217, mR, | | | |
| complete cds | | | |
| Mus musculus, Similar to hypothetical protein FLJ10520, clone | FLJ10520 | a | 0.0005 |
| MGC: 27888 IMAGE: 3497792, mR, complete cds | | | |
| Mus musculus, Similar to hypothetical protein FLJ12618, clone | FLJ12618 | a | 0.0013 |
| MGC: 28775 IMAGE: 4487011, mR, complete cds | | | |
| Mus musculus, Similar to hypothetical protein FLJ13213, clone | FLJ13213 | a | 0.0063 |
| MGC: 28555 IMAGE: 4206928, mR, complete cds | | | |
| Mus musculus, Similar to hypothetical protein FLJ20234, clone | FLJ20234 | b | |
| MGC: 37525 IMAGE: 4986113, mR, complete cds | | | |
| Mus musculus, Similar to hypothetical protein FLJ20245, clone | FLJ20245 | b | |
| MGC: 7940 IMAGE: 3584061, mR, complete cds | | | |
| Mus musculus, Similar to hypothetical protein FLJ20335, clone | D14Ertd813e | a | 0.0079 |
| MGC: 28912 IMAGE: 4922274, mR, complete cds | | | |
| Mus musculus, Similar to hypothetical protein FLJ21634, clone | FLJ21634 | * | 0.0012 |
| MGC: 19374 IMAGE: 2631696, mR, complete cds | | | |
| Mus musculus, Similar to hypothetical protein MGC3133, | SF3b10 | a | 0.006 |
| clone MGC: 11596 IMAGE: 3965951, mR, complete cds | | | |
| Mus musculus, Similar to hypothetical protein MGC4368, | MGC4368 | b | |
| clone MGC: 28978 IMAGE: 4503381, mR, complete cds | | | |
| Mus musculus, Similar to KIAA0763 gene product, clone | KIAA0763 | a | 0.0013 |
| IMAGE: 4503056, mR, partial cds | | | |
| Mus musculus, Similar to KIAA1075 protein, clone | TENC1 | * | 0.0016 |
| IMAGE: 5099327, mR, partial cds | | | |
| Mus musculus, Similar to MIPP65 protein, clone MGC: 18783 | 1500032D16Rik | a | 0.0021 |
| IMAGE: 4188234, mR, complete cds | | | |
| Mus musculus, Similar to nucleolar cysteine-rich protein, clone | HSA6591 | b | |
| MGC: 6718 IMAGE: 3586161, mR, complete cds - pending | | | |
| Mus musculus, Similar to Protein P3, clone MGC: 38638 | DXS253E | b | |
| IMAGE: 5355849, mR, complete cds | | | |
| Mus musculus, similar to quinone reductase-like protein, clone | VAT1 | a | 0.0005 |
| IMAGE: 4972406, mR, partial cds | | | |
| Mus musculus, similar to R29893_1, clone MGC: 37808 | | a | 0.0008 |
| IMAGE: 5098192, mR, complete cds | | | |
| Mus musculus, Similar to RAS p21 protein activator, clone | LOC218397 | a | 0.0009 |
| MGC: 7759 IMAGE: 3498774, mR, complete cds | | | |
| Mus musculus, Similar to retinol dehydrogese type 6, clone | RODH-4 | a | 0.0005 |
| MGC: 25965 IMAGE: 4239862, mR, complete cds | | | |
| Mus musculus, Similar to ribosomal protein S20, clone | | b | |
| MGC: 6876 IMAGE:2651405, mR, complete cds | | | |
| Mus musculus, Similar to sirtuin silent mating type information | SIRT7 | a | 0.0096 |
| regulation 2 homolog 7 (S. cerevisiae), clone MGC: 37560 | | | |
| IMAGE: 4987746, mR, complete cds | | | |
| Mus musculus, Similar to transgelin 2, clone MGC: 6300 | TAGLN2 | * | 0.0005 |
| IMAGE: 2654381, mR, complete cds | | | |
| Mus musculus, Similar to ubiquitin-conjugating enzyme E2 | UBE2V1 | * | 0.0013 |
| variant 1, clone MGC: 7660 IMAGE: 3496088, mR, complete | | | |
| cds | | | |
| Mus musculus, Similar to unc93 (C. elegans) homolog B, clone | UNC93B1 | b | |
| MGC: 25627 IMAGE: 4209296, mR, complete cds | | | |
| Mus musculus, Similar to xylulokise homolog (H. influenzae), | | * | 0.0012 |
| clone IMAGE: 5043428, mR, partial cds | | | |
| mutS homolog 2 (E. coli) | MSH2 | a | 0.0324 |
| mutS homolog 6 (E. coli) | MSH6 | a | 0.0012 |
| MYB binding protein (P160) 1a | MYBBP1A | a | 0.0005 |
| MYC-associated zinc finger protein (purine-binding | MAZ | a | 0.0031 |
| transcription factor) | | | |
| myelocytomatosis oncogene | MYC | * | 0.0012 |
| myeloid differentiation primary response gene 88 | MYD88 | b | |
| myeloid-associated differentiation marker | MYADM | a | 0.0005 |
| myocyte enhancer factor 2A | MEF2A | b | |
| myosin Ic | MYO1C | a | 0.0047 |
| myosin light chain, alkali, cardiac atria | MYL4 | a | 0.0005 |
| myosin light chain, alkali, nonmuscle | MYL6 | b | |
| myristoylated alanine rich protein kise C substrate | MACS | b | |
| N-acetylglucosamine kise | NAGK | a | 0.0083 |
| N-acetylneuramite pyruvate lyase | C1orf13 | a | 0.0068 |
| NCK-associated protein 1 | NCKAP1 | b | |
| nestin - pendin | NES | a | 0.0308 |
| neural precursor cell expressed, developmentally down- | NEDD4 | b | |
| regulated gene 4a | | | |
| neural proliferation, differentiation and control gene 1 | NPDC1 | * | 0.0042 |
| neurol guanine nucleotide exchange factor | NGEF | a | 0.0119 |
| neuropilin | NRP1 | b | |
| neutrophil cytosolic factor 2 | NCF2 | a | 0.0424 |
| Ngfi-A binding protein 2 | NAB2 | b | |
| nicotimide nucleotide transhydrogese | NNT | * | 0.0047 |
| nidogen 1 | NID | b | |
| NIMA (never in mitosis gene a)-related expressed kise 6 | NEK6 | a | 0.0012 |
| N-myc downstream regulated 2 | NDRG2 | * | 0.0005 |
| non-catalytic region of tyrosine kise adaptor protein 1 | NCK1 | b | |
| nuclear factor of kappa light chain gene enhancer in B-cells 1, | NFKB1 | b | |
| p105 | | | |
| nuclear protein 15.6 | P17.3 | a | 0.0416 |
| nuclear receptor coactivator 4 | NCOA4 | b | |
| nuclear receptor subfamily 2, group F, member 2 | NR2F2 | b | |
| nuclear receptor subfamily 2, group F, member 6 | NR2F6 | b | |
| nuclease sensitive element binding protein 1 | NSEP1 | a | 0.0005 |
| nucleophosmin 1 | NPM1 | * | 0.0032 |
| numb gene homolog (Drosophila) | NUMB | a | 0.0005 |
| oncostatin receptor | OSMR | * | 0.0021 |
| opioid growth factor receptor | OGRF | a | 0.0207 |
| ornithine aminotransferase | OAT | b | |
| ornithine decarboxylase, structural | ODC1 | a | 0.0032 |
| osteomodulin | OMD | a | 0.025 |
| oxysterol binding protein-like 1A | OSBPL1A | * | 0.0481 |
| pantophysin | HLF | * | 0.0008 |
| papillary rel cell carcinoma (translocation-associated) | PRCC | b | |
| parvalbumin | PVALB | a | 0.0026 |
| PC4 and SFRS1 interacting protein 2 (expressed sequence | PSIP2 | a | 0.0431 |
| AU015605) | | | |
| PCTAIRE-motif protein kise 3 | PCTK3 | a | 0.0396 |
| peptidylprolyl isomerase (cyclophilin)-like 1 | PPIL1 | a | 0.0424 |
| peptidylprolyl isomerase C | PPIC | a | 0.0031 |
| peptidylprolyl isomerase C-associated protein | LGALS3BP | b | |
| period homolog 1 (Drosophila) | PER1 | (b + b) = b | |
| period homolog 2 (Drosophila) | PER2 | b | |
| peroxiredoxin 5 | PRDX5 | a | 0.009 |
| peroxisomal biogenesis factor 13 | PEX13 | a | 0.0031 |
| peroxisomal delta3, delta2-enoyl-Coenzyme A isomerase | PECI | a | 0.004 |
| peroxisomal membrane protein 2, 22 kDa | PXMP2 | a | 0.0008 |
| peroxisomal sarcosine oxidase | PIPOX | a | 0.0147 |
| peroxisome proliferator activated receptor alpha | PPARA | a | 0.0018 |
| PH domain containing protein in reti 1 | PHRET1 | a | 0.0005 |
| phenylalanine hydroxylase | PAH | * | 0.0033 |
| phenylalkylamine Ca2+ antagonist (emopamil) binding protein | EBP | a | 0.0023 |
| phorbol-12-myristate-13-acetate-induced protein 1 | PMAIP1 | * | 0.0026 |
| phosphatidylinositol 3-kise, regulatory subunit, polypeptide 1 | PIK3R1 | a | 0.0381 |
| (p85 alpha) | | | |
| phosphatidylinositol transfer protein | PITPN | a | 0.0008 |
| phosphodiesterase 1A, calmodulin-dependent | PDE1A | a | 0.0361 |
| phosphofructokise, liver, B-type | PFKL | a | 0.0482 |
| phosphoglycerate kise 1 | PGK1 | a | 0.0403 |
| phosphoglycerate mutase 2 | PGAM2 | * | 0.0005 |
| phospholipase A2, activating protein | PLAA | a | 0.03 |
| phospholipase A2, group IB, pancreas | PLA2G1B | a | 0.0027 |
| phospholipase A2, group IIA (platelets, synovial fluid) | PLA2G2A | a | 0.0017 |
| phospholipid scramblase 1 | PLSCR1 | a | 0.0005 |
| phosphoprotein enriched in astrocytes 15 | PEA15 | a | 0.0008 |
| phytanoyl-CoA hydroxylase | PHYH | a | 0.0012 |
| plasminogen activator, tissue | PLAT | b | |
| platelet derived growth factor receptor, beta polypeptide | PDGFRB | a | 0.0026 |
| platelet derived growth factor, alpha | PDGFA | b | |
| platelet derived growth factor, B polypeptide | PDGFB | b | |
| platelet factor 4 | PF4 | * | 0.0018 |
| platelet-activating factor acetylhydrolase, isoform 1b, alpha1 | PAFAH1B3 | b | |
| subunit | | | |
| poliovirus receptor-related 3 | PVRL3 | (a + a) = a | 0.03; 0.0337 |
| poly (A) polymerase alpha | PAPOLA | * | 0.001 |
| poly(rC) binding protein 1 | PCBP1 | a | 0.0472 |
| polycystic kidney disease 1 homolog | PKD1 | a | 0.0316 |
| polymerase, gamma | POLG | b | |
| polypyrimidine tract binding protein 1 | PTBP1 | a | 0.0381 |
| potassium channel, subfamily K, member 2 | KCNK2 | a | 0.0096 |
| PPAR gamma coactivator-1beta protein | PERC | a | 0.0029 |
| prion protein | PRNP | b | |
| procollagen lysine, 2-oxoglutarate 5-dioxygese 2 | PLOD2 | a | 0.001 |
| procollagen, type I, alpha 1 | COL1A1 | b | |
| procollagen, type I, alpha 2 | COL1A2 | b | |
| procollagen, type IV, alpha 1 | COL4A1 | * | 0.0005 |
| procollagen, type IV, alpha 2 | COL4A2 | b | |
| procollagen, type V, alpha 1 | COL5A1 | a | 0.0017 |
| procollagen, type V, alpha 2 | COL5A2 | * | 0.0005 |
| prohibitin | PHB | a | 0.0165 |
| proline dehydrogese | PRODH | * | 0.0018 |
| protease (prosome, macropain) 26S subunit, ATPase 1 | PSMC1 | a | 0.0047 |
| proteaseome (prosome, macropain) 28 subunit, 3 | PSME3 | a | 0.0014 |
| proteasome (prosome, macropain) 26S subunit, non-ATPase, | PSMD10 | a | 0.0422 |
| 10 | | | |
| proteasome (prosome, macropain) 26S subunit, non-ATPase, | PSMD13 | a | 0.0086 |
| 13 | | | |
| proteasome (prosome, macropain) 28 subunit, alpha | PSME1 | * | 0.0012 |
| proteasome (prosome, macropain) subunit, alpha type 2 | PSMA2 | a | 0.0009 |
| proteasome (prosome, macropain) subunit, alpha type 6 | PSMA6 | a | 0.0248 |
| proteasome (prosome, macropain) subunit, alpha type 7 | PSMA7 | b | |
| proteasome (prosome, macropain) subunit, beta type 1 | PSMB1 | b | |
| proteasome (prosome, macropain) subunit, beta type 10 | PSMB10 | b | |
| protein C | PROC | a | 0.0014 |
| protein kise C, delta | PRKCD | b | |
| protein phosphatase 1, catalytic subunit, alpha isoform | PPP1CA | a | 0.0005 |
| protein phosphatase 1, regulatory (inhibitor) subunit 1A | PPP1R1A | a | 0.0005 |
| protein phosphatase 2a, catalytic subunit, beta isoform | PPP2CB | a | 0.0014 |
| protein phosphatase 3, catalytic subunit, gamma isoform | PPP3CC | a | 0.0086 |
| protein S (alpha) | PROS1 | b | |
| protein tyrosine phosphatase 4a1 | PTP4A1 | a | 0.004 |
| protein tyrosine phosphatase, non-receptor type 9 | PTPN9 | * | 0.0454 |
| protein tyrosine phosphatase, receptor type, B | PTPRB | a | 0.0497 |
| protein tyrosine phosphatase, receptor type, C | PTPRC | * | 0.0481 |
| protein tyrosine phosphatase, receptor type, C polypeptide- | PTPRCAP | b | |
| associated protein | | | |
| protein tyrosine phosphatase, receptor type, O | PTPRO | b | |
| proteoglycan, secretory granule | PRG1 | a | 0.0005 |
| proteosome (prosome, macropain) subunit, beta type 8 (large | PSMB8 | b | |
| multifunctiol protease 7) | | | |
| prothymosin alpha | PTMA | * | 0.005 |
| purinergic receptor (family A group 5); RIKEN cD | P2RY5 | b | |
| 2610302I02 gene | | | |
| pyridoxal (pyridoxine, vitamin B6) kise | PDXK | a | 0.0096 |
| PYRIN-containing APAF1-like protein 5/expressed sequence | PYPAF5 | b | |
| AI504961 | | | |
| pyruvate decarboxylase | PC | b | |
| pyruvate dehydrogese 2 | PDK2 | a | 0.0005 |
| pyruvate kise 3 | PKM2 | a | 0.0005 |
| pyruvate kise liver and red blood cell | PKLR | * | 0.031 |
| R binding motif protein 3 | RBM3 | * | 0.0005 |
| R polymerase I associated factor, 53 kD | PAF53 | a | 0.0012 |
| R polymerase II 1 | POLR2A | a | 0.0497 |
| RAB11a, member RAS oncogene family | RAB11A | a | 0.0086 |
| RAB3D, member RAS oncogene family | RAB3D | b | |
| Ral-interacting protein 1 | RALBP1 | a | 0.0063 |
| RAN, member RAS oncogene family | RAN | a | 0.0005 |
| Rap1, GTPase-activating protein 1 | RAP1GA1 | a | 0.0023 |
| RAR-related orphan receptor alpha | RORA | b | |
| ras homolog 9 (RhoC) | ARHC | * | 0.0005 |
| ras homolog B (RhoB) | ARHB | * | 0.0202 |
| ras homolog D (RhoD) | ARHD | b | |
| ras homolog gene family, member E | ARHE | a | 0.0023 |
| Ras-GTPase-activating protein (GAP<120>) SH3-domain | G3BP2 | a | 0.03 |
| binding protein 2 | | | |
| RAS-related C3 botulinum substrate 2 | RAC2 | b | |
| reduced expression 3 | BEX1 | b | |
| regulator for ribosome resistance homolog (S. cerevisiae) | RRS1 | a | 0.0013 |
| regulator of G-protein sigling 14 | RGS14 | * | 0.0018 |
| regulator of G-protein sigling 19 interacting protein 1 | RGS19IP1 | a | 0.0068 |
| renin 2 tandem duplication of Ren1 | Ren2 | b | |
| reticulocalbin | RCN1 | a | 0.0009 |
| reticulon 3 | RTN3 | a | 0.0096 |
| retinoblastoma binding protein 4 | RBBP4 | b | |
| retinoblastoma binding protein 7 | RBBP7 | a | 0.0005 |
| retinoblastoma-like 1 (p107) | RBL1 | a | 0.0057 |
| retinoic acid early transcript gamma | | b | |
| retinoic acid induced 1 | RAI1 | a | 0.0111 |
| retinol binding protein 1, cellular | RBP1 | b | |
| Rhesus blood group-associated C glycoprotein | RHCG | a | 0.0064 |
| Rho guanine nucleotide exchange factor (GEF) 3 | ARHGEF3 | a | 0.0023 |
| ribonucleotide reductase M1 | RRM1 | a | 0.0037 |
| ribosomal protein L10A | RPL10A | * | 0.0005 |
| ribosomal protein L12 | RPL12 | b | |
| ribosomal protein L13a | RPL13A | a | 0.0005 |
| ribosomal protein L18 | RPL18 | b | |
| ribosomal protein L19 | RPL19 | * | 0.0005 |
| ribosomal protein L21 | RPL21 | a | 0.0005 |
| ribosomal protein L27a | RPL27A | * | 0.0008 |
| ribosomal protein L28 | RPL28 | a | 0.0012 |
| ribosomal protein L29 | RPL29 | * | 0.0005 |
| ribosomal protein L3 | RPL3 | * | 0.0006 |
| ribosomal protein L35 | RPL35 | * | 0.0009 |
| ribosomal protein L36 | RPL36 | a | 0.0005 |
| ribosomal protein L41 | RPL41 | a | 0.0005 |
| ribosomal protein L44 | RPL36A | * | 0.0011 |
| ribosomal protein L5 | RPL5 | * | 0.0005 |
| ribosomal protein L6 | RPL6 | * | 0.0005 |
| ribosomal protein L7 | RPL7 | b | |
| ribosomal protein L8 | RPL8 | a | 0.0182 |
| ribosomal protein S14 | RPS14 | b | |
| ribosomal protein S15 | SYN1 | * | 0.0005 |
| ribosomal protein S15 | RPS15 | a | 0.0009 |
| ribosomal protein S16 | RPS16 | * | 0.0005 |
| ribosomal protein S19 | RPS19 | a | 0.0005 |
| ribosomal protein S2 | RPS2 | a | 0.0008 |
| ribosomal protein S23 | RPS23 | * | 0.0006 |
| ribosomal protein S26 | RPS26 | a | 0.0017 |
| ribosomal protein S29 | RPS29 | b | |
| ribosomal protein S3 | RPS3 | a | 0.0009 |
| ribosomal protein S3a | RPS3A | * | 0.0005 |
| ribosomal protein S4, X-linked | RPS4X | * | 0.0005 |
| ribosomal protein S5 | RPS5 | b | |
| ribosomal protein S6 | RPS6 | (* + *) = * | 0.0005; |
| | | | 0.0005 |
| ribosomal protein S6 kise, 90 kD, polypeptide 4 | RPS6KA4 | a | 0.0211 |
| ribosomal protein S7 | RPS7 | * | 0.0005 |
| ribosomal protein, large P2 | RPLP2 | b | |
| ribosomal protein, large, P1 | RPLP1 | * | 0.0005 |
| RIKEN cD 0610006F02 gene | DKFZP566H073 | (b + b) = b | |
| RIKEN cD 0610006N12 gene | NDUFB4 | a | 0.0163 |
| RIKEN cD 0610007L01 gene | FLJ10099 | a | 0.008 |
| RIKEN cD 0610011C19 gene | FLJ22386 | a | 0.0077 |
| RIKEN cD 0610016J10 gene | CGI-27 | a | 0.0014 |
| RIKEN cD 0610025G13 gene | RPL38 | * | 0.0023 |
| RIKEN cD 0610025I19 gene | 0610025I19Rik | * | 0.0005 |
| RIKEN cD 0610041E09 gene | AD-020 | a | 0.0042 |
| RIKEN cD 1010001M04 gene | 1010001M04Rik | a | 0.0005 |
| RIKEN cD 1100001F19 gene | UBE2D3 | a | 0.0048 |
| RIKEN cD 1100001J13 gene - pending | KIAA1049 | a | 0.0296 |
| RIKEN cD 1110001I24 gene | BZW2 | * | 0.0025 |
| RIKEN cD 1110002C08 gene | MGC9564 | a | 0.0497 |
| RIKEN cD 1110005N04 gene | TAF5L | b | |
| RIKEN cD 1110007F23 gene | 1110007F23Rik | b | |
| RIKEN cD 1110008B24 gene | C14orf111 | b | |
| RIKEN cD 1110014C03 gene | TMP21 | a | 0.0008 |
| RIKEN cD 1110020L19 gene | TREX2 | a | 0.0422 |
| RIKEN cD 1110032A13 gene | FLJ21172 | b | |
| RIKEN cD 1110038J12 gene | | * | 0.0068 |
| RIKEN cD 1110038L14 gene | CKS2 | a | 0.0086 |
| RIKEN cD 1110054A24 gene | 1110054A24Rik | a | 0.0335 |
| RIKEN cD 1190006C12 gene | SEC61B | b | |
| RIKEN cD 1200003E16 gene | 1200003E16Rik | a | 0.004 |
| RIKEN cD 1200009B18 gene | LOC51290 | b | |
| RIKEN cD 1200011D11 gene | BK65A6.2 | a | 0.0005 |
| RIKEN cD 1200013A08 gene | MGC3047 | b | |
| RIKEN cD 1200014D15 gene | DMGDH | * | 0.0006 |
| RIKEN cD 1200014I03 gene | F13A1 | a | 0.0015 |
| RIKEN cD 1200015A22 gene | MGC3222 | a | 0.0119 |
| RIKEN cD 1200016G03 gene | 1200016G03Rik | a | 0.0012 |
| RIKEN cD 1300002P22 gene | ECH1 | a | 0.0013 |
| RIKEN cD 1300004O04 gene | CACH-1 | * | 0.0068 |
| RIKEN cD 1300013F15 gene | FLJ22390 | b | |
| RIKEN cD 1300013G12 gene | 1300013G12Rik | a | 0.0072 |
| RIKEN cD 1300017C12 gene | FLJ10948 | a | 0.0011 |
| RIKEN cD 1300018I05 gene | KIAA0082 | a | 0.0472 |
| RIKEN cD 1300019I21 gene | MTAP | a | 0.0012 |
| RIKEN cD 1500010B24 gene | EIF1A | (b + b) = b | |
| RIKEN cD 1500026A19 gene | ALG5 | a | 0.0189 |
| RIKEN cD 1500041J02 gene | FLJ13448 | * | 0.0497 |
| RIKEN cD 1700008H23 gene | 1700008H23Rik | b | |
| RIKEN cD 1700012B18 gene | OKL38 | a | 0.0381 |
| RIKEN cD 1700015P13 gene | 1700015P13Rik | b | |
| RIKEN cD 1700016A15 gene | FLJ11806 | b | |
| RIKEN cD 1700028A24 gene | LOC55862 | a | 0.0096 |
| RIKEN cD 1700037H04 gene | FLJ20550 | a | 0.0381 |
| RIKEN cD 1810009M01 gene | LR8 | a | 0.0005 |
| RIKEN cD 1810013B01 gene | 1810013B01Rik | a | 0.0015 |
| RIKEN cD 1810023B24 gene | FLJ14503 | a | 0.0424 |
| RIKEN cD 1810027P18 gene | DCXR | a | 0.0013 |
| RIKEN cD 1810036E22 gene | | a | 0.004 |
| RIKEN cD 1810038D15 gene | DKFZP566E144 | a | 0.0096 |
| RIKEN cD 1810043O07 gene | KIAA0601 | b | |
| RIKEN cD 1810054O13 gene | 1810054O13Rik | a | 0.0005 |
| RIKEN cD 1810058K22 gene | CDC42EP1 | a | 0.0009 |
| RIKEN cD 2010012D11 gene | 2010012D11Rik | * | 0.0065 |
| RIKEN cD 2010315L10 gene | MDS032 | a | 0.006 |
| RIKEN cD 2310001A20 gene | C20orf3 | a | 0.0012 |
| RIKEN cD 2310004I03 gene | 2310004I03Rik | a | 0.0482 |
| RIKEN cD 2310004L02 gene | FLJ10241 | * | 0.0006 |
| RIKEN cD 2310009E04 gene | FLJ10986 | * | 0.0005 |
| RIKEN cD 2310010G13 gene | 2310010G13Rik | a | 0.025 |
| RIKEN cD 2310022K15 gene | KLHDC2 | b | |
| RIKEN cD 2310032J20 gene | BDH | a | 0.0032 |
| RIKEN cD 2310046G15 gene | SPUVE | b | |
| RIKEN cD 2310051E17 gene | 2310051E17Rik | a | 0.0005 |
| RIKEN cD 2310067B10 gene | KIAA0195 | a | 0.0452 |
| RIKEN cD 2310075M15 gene | 2310075M15Rik | (a + *) = * | 0.0099 |
| RIKEN cD 2310079C17 gene | DKFZP547E2110 | a | 0.0154 |
| RIKEN cD 2410002J21 gene | ENIGMA | a | 0.0309 |
| RIKEN cD 2410021P16 gene | MGC5601 | a | 0.0012 |
| RIKEN cD 2410026K10 gene | CD99 | b | |
| RIKEN cD 2410029D23 gene | ATP6V1E1 | a | 0.0162 |
| RIKEN cD 2410129E14 gene | | b | |
| RIKEN cD 2410174K12 gene | SUGT1 | b | |
| RIKEN cD 2510015F01 gene | FLJ12442 | a | 0.0005 |
| RIKEN cD 2600001N01 gene | ZWINT | a | 0.0013 |
| RIKEN cD 2600015J22 gene | | b | |
| RIKEN cD 2600017H24 gene | | a | 0.0331 |
| RIKEN cD 2610007A16 gene | SEC13L | a | 0.0005 |
| RIKEN cD 2610029K21 gene | FLJ20249 | a | 0.0126 |
| RIKEN cD 2610039E05 gene | 2610039E05Rik | a | 0.0046 |
| RIKEN cD 2610200M23 gene | SSBP3 | b | |
| RIKEN cD 2610206D03 gene | 2610206D03Rik | a | 0.0018 |
| RIKEN cD 2610301D06 gene | 2610301D06Rik | a | 0.0005 |
| RIKEN cD 2610305D13 gene | FLJ11191 | a | 0.0009 |
| RIKEN cD 2610306D21 gene | ANAPC4 | b | |
| RIKEN cD 2610511O17 gene | FLJ20272 | a | 0.0157 |
| RIKEN cD 2610524G07 gene | | a | 0.0013 |
| RIKEN cD 2610524G09 gene | IER5 | a | 0.0491 |
| RIKEN cD 2700027J02 gene | SPF45 | a | 0.0243 |
| RIKEN cD 2700038K18 gene | | b | |
| RIKEN cD 2700038M07 gene - pending | WSB1 | b | |
| RIKEN cD 2700055K07 gene | CGI-38 | b | |
| RIKEN cD 2700099C19 gene | LOC51248 | a | 0.0057 |
| RIKEN cD 2810004N23 gene | 2810004N23Rik | a | 0.0073 |
| RIKEN cD 2810047L02 gene | RAMP | a | 0.004 |
| RIKEN cD 2810409H07 gene | PTD004 | a | 0.0018 |
| RIKEN cD 2810411G23 gene | TPD52L2 | a | 0.0026 |
| RIKEN cD 2810418N01 gene | KIAA0186 | b | |
| RIKEN cD 2810430J06 gene | FRCP1 | b | |
| RIKEN cD 2810468K17 gene | MGC13272 | b | |
| RIKEN cD 2810473M14 gene | 2810473M14Rik | a | 0.0139 |
| RIKEN cD 2900074L19 gene | | b | |
| RIKEN cD 3010001A07 gene | BFAR | a | 0.0244 |
| RIKEN cD 3010027G13 gene | DKFZp434C119.1 | a | 0.0008 |
| RIKEN cD 3021401A05 gene | 3021401A05Rik | * | 0.006 |
| RIKEN cD 3110001N18 gene | RPL22 | b | |
| RIKEN cD 3230402E02 gene | FLJ10983 | a | 0.0201 |
| RIKEN cD 3321401G04 gene | KIAA0738 | b | |
| RIKEN cD 4430402G14 gene | H3f3b | * | 0.0012 |
| RIKEN cD 4632401C08 gene | 4632401C08Rik | a | 0.0005 |
| RIKEN cD 4733401N12 gene | CPSF6 | b | |
| RIKEN cD 4921528E07 gene | 4921528E07Rik | b | |
| RIKEN cD 4921537D05 gene | NY-REN-58 | a | 0.033 |
| RIKEN cD 4930506M07 gene | FLJ11122 | a | 0.03 |
| RIKEN cD 4930533K18 gene | | * | 0.0005 |
| RIKEN cD 4930542G03 gene | 4930542G03Rik | a | 0.0005 |
| RIKEN cD 4930552N12 gene | MCCC2 | * | 0.0009 |
| RIKEN cD 4930579A11 gene | VMP1 | a | 0.0023 |
| RIKEN cD 4932442K08 gene | 4932442K08Rik | b | |
| RIKEN cD 4933405K01 gene | MGC14799 | a | 0.0037 |
| RIKEN cD 5031412I06 gene | Dutp | a | 0.0068 |
| RIKEN cD 5031422I09 gene | PKP4 | * | 0.0023 |
| RIKEN cD 5133400A03 gene | 5133400A03Rik | * | 0.0005 |
| RIKEN cD 5133401H06 gene | 5133401H06Rik | a | 0.0008 |
| RIKEN cD 5430416A05 gene | AD034 | a | 0.024 |
| RIKEN cD 5630401J11 gene | 5630401J11Rik | b | |
| RIKEN cD 5730403B10 gene | C16orf5 | a | 0.0092 |
| RIKEN cD 5730406I15 gene | KIAA0102 | b | |
| RIKEN cD 5730534O06 gene | KIAA0164 | a | 0.0006 |
| RIKEN cD 5830445O15 gene | 5830445O15Rik | a | 0.0119 |
| RIKEN cD 6230410I01 gene | FLJ10849 | b | |
| RIKEN cD 6330565B14 gene | ADH8 | * | 0.0009 |
| RIKEN cD 6330583M11 gene | DKFZP434P106 | * | 0.0005 |
| RIKEN cD 6430559E15 gene | HT036 | a | 0.0008 |
| RIKEN cD 6530411B15 gene | DKFZp564K1964.1 | * | 0.0086 |
| RIKEN cD 6720463E02 gene | | a | 0.0047 |
| RIKEN cD 9130011J04 gene | 9130011J04Rik | b | |
| RIKEN cD 9130022E05 gene | 9130022E05Rik | a | 0.0353 |
| RIKEN cD 9530058B02 gene | MGC15416 | * | 0.0005 |
| RIKEN cD 9530089B04 gene | 9530089B04Rik | * | 0.0023 |
| RIKEN cD A230106A15 gene | A230106A15Rik | a | 0.0424 |
| RIKEN cD A330103N21 gene | A330103N21Rik | (a + a) = a | 0.0012; |
| | | | 0.0072 |
| RIKEN cD A930008K15 gene | KIAA0605 | a | 0.0054 |
| RIKEN cD D630002J15 gene | D630002J15Rik | a | 0.0068 |
| RIKEN cD E130113K08 gene | T50835 | b | |
| ring finger protein (C3HC4 type) 19 | RNF19 | b | |
| runt related transcription factor 1 | RUNX1 | b | |
| S100 calcium binding protein A10 (calpactin) | S100A10 | * | 0.0005 |
| S100 calcium binding protein A13 | S100A13 | b | |
| S100 calcium binding protein A4 | S100A4 | * | 0.0026 |
| S100 calcium binding protein A6 (calcyclin) | S100A6 | * | 0.0005 |
| S-adenosylhomocysteine hydrolase | AHCY | b | |
| SAR1a gene homolog (S. cerevisiae) | SAR1 | a | 0.0018 |
| schlafen 4 | FLJ10260 | a | 0.0023 |
| SEC13 related gene (S. cerevisiae) RIKEN cD 1110003H02 | SEC13L1 | a | 0.0096 |
| gene | | | |
| SEC61, gamma subunit (S. cerevisiae) | SEC61G | a | 0.0081 |
| secreted acidic cysteine rich glycoprotein | SPARC | * | 0.0005 |
| secreted and transmembrane 1 | SECTM1 | b | |
| secreted phosphoprotein 1 | SPP1 | a | 0.0005 |
| selectin, platelet (p-selectin) ligand | SELPLG | b | |
| selenium binding protein 2 | SELENBP1 | b | |
| selenophosphate synthetase 2 | SPS2 | b | |
| selenoprotein P, plasma, 1 | SEPP1 | a | 0.0086 |
| septin 8 | KIAA0202 | a | 0.025 |
| serine (or cysteine) proteise inhibitor, clade B (ovalbumin), | SERPINB2 | a | 0.0013 |
| member 2 | | | |
| serine (or cysteine) proteise inhibitor, clade E (nexin, | SERPINE2 | b | |
| plasminogen activator inhibitor type 1), member 2 | | | |
| serine (or cysteine) proteise inhibitor, clade G (C1 inhibitor), | SERPING1 | b | |
| member 1 | | | |
| serine (or cysteine) proteise inhibitor, clade H (heat shock | SERPINH1 | * | 0.0005 |
| protein 47), member 1 | | | |
| serine hydroxymethyl transferase 1 (soluble) | SHMT1 | b | |
| serine hydroxymethyl transferase 2 (mitochondrial); RIKEN | SHMT2 | * | 0.0005 |
| cD 2700043D08 gene | | | |
| serine palmitoyltransferase, long chain base subunit 1 | SPTLC1 | a | 0.0422 |
| serine protease inhibitor 6 | SERPINB9 | b | |
| serine protease inhibitor, Kunitz type 1 | SPINT1 | a | 0.0011 |
| serine protease inhibitor, Kunitz type 2 | SPINT2 | a | 0.0071 |
| serine/arginine repetitive matrix 1 | RAD23B | a | 0.0068 |
| serine/threonine kise receptor associated protein | UNRIP | a | 0.0119 |
| serine/threonine protein kise CISK | SGKL | a | 0.0424 |
| serum amyloid A 3 | SAA3P | a | 0.0008 |
| serum/glucocorticoid regulated kise | SGK | b | |
| serum/glucocorticoid regulated kise 2 | SGK2 | * | 0.0006 |
| SET translocation | SET | a | 0.005 |
| sex-lethal interactor homolog (Drosophila) | RPC5 | * | 0.0058 |
| SFFV proviral integration 1 | SPI1 | b | |
| SH3 domain binding glutamic acid-rich protein-like 3 | SH3BGRL3 | * | 0.0005 |
| SH3 domain protein 3 | OSTF1 | a | 0.0037 |
| sideroflexin 1 | SFXN1 | a | 0.0201 |
| sigl sequence receptor, delta | SSR4 | * | 0.0023 |
| sigl transducer and activator of transcription 3 | STAT3 | b | |
| sigling intermediate in Toll pathway-evolutiorily conserved | Sitpec | b | |
| single Ig IL-1 receptor related protein | SIGIRR | b | |
| slit homolog 2 (Drosophila) | SLIT2 | a | 0.0057 |
| slit homolog 3 (Drosophila) | SLIT3 | b | |
| small inducible cytokine A2 | SCYA2 | * | 0.0008 |
| small inducible cytokine A5 | SCYA5 | b | |
| small inducible cytokine A7 | SCYA7 | b | |
| small inducible cytokine A9 | CCL9 | * | 0.0016 |
| small inducible cytokine B subfamily (Cys-X-Cys), member 10 | SCYB10 | * | 0.0005 |
| small inducible cytokine B subfamily, member 5 | SCYB6 | b | |
| small inducible cytokine subfamily D, 1 | SCYD1 | * | 0.0091 |
| small nuclear ribonucleoprotein D2 | SNRPD2 | * | 0.0116 |
| small nuclear ribonucleoprotein E | SNRPE | b | |
| small nuclear ribonucleoprotein polypeptide G | SNRPG | * | 0.0042 |
| small proline-rich protein 1A | SPRR1A | b | |
| SMC (structural maintence of chromosomes 1)-like 1 (S. cerevisiae) | SMC1L1 | a | 0.0018 |
| smoothelin | SMTN | a | 0.0005 |
| smoothened homolog (Drosophila) | SMOH | b | |
| soc-2 (suppressor of clear) homolog (C. elegans) | SHOC2 | b | |
| solute carrier family 1, member 1 | SLC1A1 | b | |
| solute carrier family 12, member 1 | SLC12A1 | a | 0.0023 |
| solute carrier family 13 (sodium/sulphate symporters), member 1 | SLC13A1 | * | 0.0021 |
| solute carrier family 13 (sodium-dependent dicarboxylate | SLC13A3 | * | 0.0047 |
| transporter), member 3 | | | |
| solute carrier family 15 (H+/peptide transporter), member 2 | SLC15A2 | a | 0.0037 |
| solute carrier family 16 (monocarboxylic acid transporters), | SLC16A2 | a | 0.0058 |
| member 2 | | | |
| solute carrier family 16 (monocarboxylic acid transporters), | SLC16A7 | b | |
| member 7 | | | |
| solute carrier family 2 (facilitated glucose transporter), member 5 | SLC2A5 | b | |
| solute carrier family 22 (organic anion transporter), member 6 | SLC22A6 | b | |
| solute carrier family 22 (organic anion transporter), member 8/ | SLC22A8 | * | 0.0005 |
| (Roct) reduced in osteosclerosis transporter | | | |
| solute carrier family 22 (organic cation transporter), member 1 | SLC22A1 | * | 0.0009 |
| solute carrier family 22 (organic cation transporter), member 1- | SLC22A1L | * | 0.0005 |
| like | | | |
| solute carrier family 22 (organic cation transporter), member 2 | SLC22A2 | * | 0.0005 |
| solute carrier family 22 (organic cation transporter), member 4 | SLC22A4 | b | |
| solute carrier family 22 (organic cation transporter), member 5 | SLC22A5 | * | 0.0015 |
| solute carrier family 22 (organic cation transporter)-like 2 | Slc22al2 | a | 0.0088 |
| solute carrier family 25 (mitochondrial carrier | SLC25A10 | a | 0.0005 |
| solute carrier family 25 (mitochondrial carrier | SLC25A13 | b | |
| solute carrier family 25 (mitochondrial deoxynucleotide | SLC25A19 | a | 0.0005 |
| carrier), member 19 | | | |
| solute carrier family 26, member 4 | SLC26A4 | * | 0.033 |
| solute carrier family 27 (fatty acid transporter), member 2 | SLC27A2 | * | 0.0146 |
| solute carrier family 3, member 1 | SLC3A1 | b | |
| solute carrier family 31, member 1 | SLC31A1 | a | 0.0206 |
| solute carrier family 34 (sodium phosphate), member 1 | SLC34A1 | a | 0.005 |
| solute carrier family 34 (sodium phosphate), member 2 | SLC34A2 | b | |
| solute carrier family 35, member A5; RIKEN cD 1010001J06 | SLC35A5 | a | 0.0026 |
| gene | | | |
| solute carrier family 4 (anion exchanger), member 4 | SLC4A4 | * | 0.0221 |
| solute carrier family 6 (neurotransmitter transporter, glycine), | SLC6A9 | a | 0.0225 |
| member 9/glycine transporter 1 | | | |
| solute carrier family 7 (cationic amino acid transporter, y+ | SLC7A7 | * | 0.025 |
| system), member 7 | | | |
| solute carrier family 7 (cationic amino acid transporter, y+ | SLC7A9 | * | 0.0008 |
| system), member 9 | | | |
| speckle-type POZ protein | SPOP | a | 0.0135 |
| spermatogenesis associated factor | SPATA5 | a | 0.0189 |
| spermidine synthase | SRM | a | 0.0026 |
| spermidine/spermine N1-acetyl transferase | SAT | b | |
| sphingomyelin phosphodiesterase 2, neutral | SMPD2 | a | 0.0047 |
| splicing factor 3b, subunit 1, 155 kDa | SF3B1 | * | 0.0162 |
| splicing factor, arginine/serine-rich 2 (SC-35) | SFRS2 | a | 0.0011 |
| split hand/foot deleted gene 1 | DSS1 | b | |
| src homology 2 domain-containing transforming protein D | SHD | a | 0.027 |
| src-like adaptor protein | SLA | a | 0.0183 |
| stearoyl-Coenzyme A desaturase 1 | SCD | * | 0.0008 |
| steroid receptor R activator 1 | SRA1 | a | 0.0012 |
| sterol carrier protein 2, liver | SCP2 | * | 0.0008 |
| striatin, calmodulin binding protein 4/expressed sequence | STRN4 | b | |
| C80611 | | | |
| stromal cell derived factor 1 | CXCL12 | a | 0.0012 |
| succinate dehydrogenase complex, subunit B, iron sulfur (Ip); | SDHB | a | 0.0011 |
| RIKEN cD 0710008N11 gene | | | |
| succite dehydrogese complex, subunit A, flavoprotein (Fp) | SDHA | a | 0.0006 |
| succite-Coenzyme A ligase, ADP-forming, beta subunit | SUCLA2 | a | 0.0015 |
| succite-Coenzyme A ligase, GDP-forming, beta subunit | SUCLG2 | a | 0.0197 |
| sulfotransferase-related protein SULT-X1 | Sult-x1 | b | |
| superoxide dismutase 2, mitochondrial | SOD2 | * | 0.0005 |
| surfeit gene 4 | SURF4 | a | 0.0058 |
| SWI/SNF related, matrix associated, actin dependent regulator | SMARCA5 | (a + a) = a | 0.0183; |
| of chromatin, subfamily a, member 5 | | | 0.0166 |
| SWI/SNF related, matrix associated, actin dependent regulator | SMARCE1 | a | 0.0013 |
| of chromatin, subfamily e, member 1 | | | |
| syndecan 1 | SDC1 | a | 0.0008 |
| syntrophin, basic 2 | SNTB2 | a | 0.0197 |
| TAF10 R polymerase II, TATA box binding protein (TBP)- | TAF10 | a | 0.0006 |
| associated factor, 30 kDa | | | |
| TAF9 R polymerase II, TATA box binding protein (TBP)- | TAF9 | a | 0.0178 |
| associated factor, 32 kDa | | | |
| talin 2 | TLN2 | * | 0.0005 |
| TATA box binding protein-like protein | TBPL1 | b | |
| T-box 6 | TBX6 | * | 0.0497 |
| T-cell specific GTPase | Tgtp | b | |
| T-cell, immune regulator 1 | TCIRG1 | b | |
| TEA domain family member 2 | TEAD2 | a | 0.0112 |
| tescin C | TNC | * | 0.0005 |
| tescin XB | TNXB | a | 0.036 |
| testis derived transcript | TES | a | 0.0018 |
| tetranectin (plasminogen binding protein) | TNA | a | 0.0204 |
| tetratricopeptide repeat domain | TTC3 | b | |
| TG interacting factor | TGIF | * | 0.006 |
| thiamin pyrophosphokise | TPK1 | a | 0.0078 |
| thioesterase, adipose associated | THEA | * | 0.0119 |
| thioether S-methyltransferase | Temt | b | |
| thioredoxin 1 | TXN | * | 0.0009 |
| thioredoxin 2 | TXN2 | b | |
| thioredoxin-like (32 kD) | TXNL | a | 0.0023 |
| thrombospondin 1 | THBS1 | b | |
| thymidine kise 1 | TK1 | a | 0.0245 |
| thymoma viral proto-oncogene 1 | AKT1 | a | 0.0005 |
| thymosin, beta 4, X chromosome | TMSB4X | * | 0.0005 |
| thyroid hormone responsive SPOT14 homolog (Rattus) | THRSP | * | 0.001 |
| Tiall cytotoxic granule-associated R binding protein-like 1 | TIAL1 | a | 0.01 |
| tight junction protein 2 | TJP2 | b | |
| tissue inhibitor of metalloproteise | TIMP1 | * | 0.0005 |
| Tnf receptor-associated factor 2 | TRAF2 | a | 0.0037 |
| toll-like receptor 2 | TLR2 | b | |
| topoisomerase (D) III beta | TOP3B | a | 0.0186 |
| TRAF-interacting protein | TRIP | a | 0.004 |
| transcobalamin 2 | TCN2 | * | 0.0012 |
| transcription elongation factor A (SII), 3 | TCEA3 | a | 0.0068 |
| transcription elongation regulator 1 (CA150) | TCERG1 | * | 0.0005 |
| transcription factor 21 | TCF21 | b | |
| transcription factor 4 | TCF4 | b | |
| transcription factor Dp 1 | TFDP1 | b | |
| transformation related protein 53 | TP53 | a | 0.0005 |
| transformed mouse 3T3 cell double minute 2 | MDM2 | b | |
| transforming growth factor beta 1 induced transcript 4 | TSC22 | * | 0.0012 |
| transforming growth factor, beta induced, 68 kDa | TGFBI | * | 0.0005 |
| transgelin | TAGLN | * | 0.0173 |
| translin | TSN | a | 0.004 |
| transmembrane 7 superfamily member 1 | TM7SF1 | a | 0.0023 |
| transmembrane protein 8 (five membrane-spanning domains) | TMEM8 | (* + a) = * | 0.0219; |
| | | | 0.0026 |
| Trans-prenyltransferase | Tprt | b | |
| transthyretin | TTR | a | 0.0086 |
| trinucleotide repeat containing 11 (THR-associated protein, 230 kDa | TNRC11 | b | |
| subunit) | | | |
| tropomyosin 2, beta | TPM2 | a | 0.0005 |
| tropomyosin 3, gamma | TPM3 | * | 0.0005 |
| tubulin alpha 1 | TUBA1 | b | |
| tubulin alpha 2 | TUBA2 | * | 0.0005 |
| tubulin, beta 5 | TUBB | a | 0.0005 |
| tuftelin 1 | TUFT1 | a | 0.004 |
| tumor necrosis factor receptor superfamily, member 10b | TNFRSF10B | a | 0.0198 |
| tumor necrosis factor receptor superfamily, member 1a | TNFRSF1A | * | 0.018 |
| tumor necrosis factor receptor superfamily, member 1b | TNFRSF1B | b | |
| tumor protein p53 binding protein, 2/expressed sequence | TP53BP2 | b | |
| AI746547 | | | |
| tumor rejection antigen gp96 | TRA1 | a | 0.0103 |
| tumor-associated calcium sigl transducer 2 | TACSTD2 | * | 0.0005 |
| tural killer tumor recognition sequence | NKTR | * | 0.0022 |
| TYRO protein tyrosine kise binding protein | TYROBP | * | 0.0008 |
| tyrosine 3-monooxygese/tryptophan 5-monooxygese activation | YWHAE | a | 0.0006 |
| protein, epsilon polypeptide | | | |
| tyrosine 3-monooxygese/tryptophan 5-monooxygese activation | YWHAH | * | 0.0005 |
| protein, eta polypeptide | | | |
| ubiquitin specific protease 2 | USP2 | * | 0.0005 |
| ubiquitin specific protease 7 (expressed sequence AA409944) | USP7 | a | 0.0005 |
| ubiquitin-conjugating enzyme E2D 2 | UBE2D2 | b | |
| ubiquitin-conjugating enzyme E2H | UBE2H | * | 0.0068 |
| ubiquitin-conjugating enzyme E2I | UBE2I | a | 0.0005 |
| ubiquitin-conjugating enzyme E2L 3 | UBE2L3 | a | 0.0072 |
| ubiquitin-conjugating enzyme E2N | UBE2N | * | 0.0009 |
| ubiquitin-like 1 | UBL1 | a | 0.0381 |
| ubiquitin-like 1 (sentrin) activating enzyme E1A | SAE1 | a | 0.004 |
| ubiquitin-like 1 (sentrin) activating enzyme E1B | UBA2 | a | 0.0011 |
| UDP-Gal:betaGlcc beta 1,3-galactosyltransferase, polypeptide 3 | B3GALT3 | a | 0.0057 |
| UDP-Gal:betaGlcc beta 1,4-galactosyltransferase, polypeptide 2 | B4GALT2 | a | 0.0005 |
| UDP-N-acetyl-alpha-D-galactosamine:(N-acetylneuraminyl)- | GALGT | * | 0.0052 |
| galactosylglucosylceramide-beta-1,4-N- | | | |
| acetylgalactosaminyltransferase | | | |
| Unknown | | * | 0.0005 |
| Unknown | ITGA5 | * | 0.0022 |
| Unknown | | * | 0.0005 |
| Unknown | | * | 0.0005 |
| Unknown | COL18A1 | (* + *) = * | 0.0005; |
| | | | 0.0009 |
| Unknown | | * | 0.006 |
| Unknown | | * | 0.0012 |
| Unknown | | * | 0.0096 |
| Unknown | | * | 0.0191 |
| Unknown | | * | 0.0367 |
| Unknown | | a | 0.0424 |
| Unknown | | a | 0.0047 |
| Unknown | | a | 0.0019 |
| Unknown | | a | 0.0005 |
| Unknown | | a | 0.01 |
| Unknown | | a | 0.0204 |
| Unknown | | a | 0.0063 |
| Unknown | | a | 0.0005 |
| Unknown | | a | 0.0079 |
| Unknown | | a | 0.0017 |
| Unknown | | a | 0.0032 |
| Unknown | | a | 0.0494 |
| Unknown | | a | 0.0009 |
| Unknown | | a | 0.0459 |
| Unknown | | a | 0.0042 |
| Unknown | | b | |
| Unknown | | b | |
| Unknown | | b | |
| Unknown | | b | |
| Unknown | | b | |
| Unknown | | b | |
| Unknown | | b | |
| Unknown | | b | |
| Unknown | | b | |
| upregulated during skeletal muscle growth 5 | USMG5 | b | |
| upstream transcription factor 1 | USF1 | a | 0.01 |
| urokise plasminogen activator receptor | PLAUR | * | 0.0042 |
| UUDP glycosyltransferase 1 family, polypeptide A6 | | b | |
| vascular cell adhesion molecule 1 | VCAM1 | b | |
| vascular endothelial growth factor A | VEGF | (a + b) = * | 0.0219 |
| vascular endothelial zinc finger 1; expressed sequence | Vezf1 | a | 0.0305 |
| AI848691 | | | |
| vasodilator-stimulated phosphoprotein | VASP | * | 0.0054 |
| vitamin D receptor | VDR | a | 0.0016 |
| v-ral simian leukemia viral oncogene homolog A (ras related) | RALA | b | |
| v-ral simian leukemia viral oncogene homolog B (ras related) | RALB | * | 0.0005 |
| WD repeat domain 1 | WDR1 | a | 0.0012 |
| Williams-Beuren syndrome chromosome region 14 homolog | WBSCR14 | a | 0.0005 |
| (human) | | | |
| WNT1 inducible sigling pathway protein 1 | WISP1 | b | |
| X (ictive)-specific transcript, antisense | TSIX | b | |
| X transporter protein 2 | Xtrp2 | b | |
| Yamaguchi sarcoma viral (v-yes) oncogene homolog | YES1 | b | |
| Yamaguchi sarcoma viral (v-yes-1) oncogene homolog | LYN | b | |
| yolk sac gene 2 | DKFZp761A051.1 | a | 0.0046 |
| zinc finger like protein 1 | ZFPL1 | b | |
| zinc finger protein 144 | ZNF144 | b | |
| zinc finger protein-36, C3H type-like 1 | ZFP36L1 | * | 0.0009 |
| zinc finger protein 36, C3H type-like 2 | ZFP36L2 | * | 0.0005 |
| zuotin related factor 2 | ZRF1 | a | 0.0118 |

| | | | | | | | Concordant |
|---|---|---|---|---|---|---|---|
| | | | | Expression of | | | (C) or |
| | | | | regeneration/normal: | | | Disconcordant |
| | | p-value | | Early(A)/Late(B)/ | | | (DC) with the |
| | fold (day 1-2 | (day 5-14 | fold (day 5-14 | both (*) Vs. | RCC/ | | renal |
| | vs Normal- | vs | vs | Normal; (Up (+); | normal | | regeneration |
| Gene name | Ischmic) | Normal) | Normal) | Down (−)) | kidney | RCC | dataset |
| (Gus-s) beta-glucuronidase structural | | 0.018 | 1.3665 | (+) | | | |
| (Prlr-rs1) prolactin receptor related | 0.438069 | 0.009 | 0.5628 | (−) | | | |
| sequence 1 | | | | | | | |
| (Sdccagg28) serologically defined | 0.767583 | | | (−) | | | |
| colon cancer antigen 28 | | | | | | | |
| ((AW146109) expressed sequence | 1.762737 | 0.006 | 1.7551 | (+) | (+) | | C |
| AW146109) | | | | | | | |
| (2610524K04Rik; RIKEN cD | 1.456446 | | | (+) | | | |
| 2610524K04 gene) | | | | | | | |
| 1-acylglycerol-3-phosphate O- | 0.741613 | | | (−) | (−) | RCC | C |
| acyltransferase 3; expressed | | | | | | | |
| sequence AW493985 | | | | | | | |
| 2′-5′ oligoadenylate synthetase 1A | 1.224876 | | | (+) | | | |
| 2-hydroxyphytanoyl-CoA lyase | | 0.003 | 0.7615 | (−) | (−) | RCC | C |
| 3-hydroxy-3-methylglutaryl- | 0.711153 | | | (−) | | | |
| Coenzyme A synthase 1 | | | | | | | |
| 3-phosphoglycerate dehydrogese | 1.523954 | | | (+) | (−)/(+) | RCC | conflict |
| 4-hydroxyphenylpyruvic acid | 0.305971 | 8E−04 | 0.3436 | (−) | (−) | RCC | C |
| dioxygese | | | | | | | |
| 5′,3′ nucleotidase, cytosolic | | 0.037 | 1.2614 | (+) | | | |
| 5-azacytidine induced gene 1 | 0.871679 | | | (−) | | | |
| a disintegrin and metalloproteise | 1.301018 | 0.018 | 1.2626 | (+) | | | |
| domain 12 (meltrin alpha) | | | | | | | |
| a disintegrin-like and metalloprotease | 2.236459 | 8E−04 | 2.0162 | (+) | | | |
| (reprolysin type) with | | | | | | | |
| thrombospondin type 1 motif, 1 | | | | | | | |
| a disintegrin-like and metalloprotease | 1.226952 | | | (+) | | | |
| (reprolysin type) with | | | | | | | |
| thrombospondin type 1 motif, 2 | | | | | | | |
| A kise (PRKA) anchor protein 2 | 1.477284 | | | (+) | (−) | RCC | DC |
| acetyl-Coenzyme A acyltransferase 2 | 0.548469 | 0.002 | 0.5885 | (−) | | | |
| (mitochondrial 3-oxoacyl-Coenzyme | | | | | | | |
| A thiolase) (D18Ertd240e) RIKEN | | | | | | | |
| cD 0610011L04 gene | | | | | | | |
| acetyl-Coenzyme A dehydrogese, | 0.377562 | | | (−) | | | |
| medium chain | | | | | | | |
| acetyl-Coenzyme A transporter | 0.750342 | | | (−) | | | |
| acidic ribosomal phosphoprotein PO | 1.814377 | | | (+) | (+) | RCC | C |
| aconitase 1 | | 0.009 | 0.7388 | (−) | (−) | RCC | C |
| actin related protein ⅔ complex, | 1.291043 | | | (+) | (+) | RCC | C |
| subunit 3 (21 kDa) | | | | | | | |
| actin, alpha 1, skeletal muscle | | 0.022 | 1.7931 | (+) | | | |
| actin, alpha 2, smooth muscle, aorta | 2.549549 | 0.003 | 1.711 | (+) | | | |
| actin, beta, cytoplasmic | 1.861028 | 0.001 | 1.9517 | (+) | (+) | RCC | C |
| actin, gamma 2, smooth muscle, | 1.48389 | 0.008 | 1.7721 | (+) | | | |
| enteric | | | | | | | |
| actin-like | 2.02784 | 0.036 | 1.7173 | (+) | | | |
| activator of S phase kise | 1.418184 | | | (+) | | | |
| activity-dependent neuroprotective | | 0.022 | 1.2684 | (+) | | | |
| protein | | | | | | | |
| acyl-Coenzyme A dehydrogese, | 0.677684 | 0.009 | 0.7072 | (−) | (−) | RCC | C |
| short/branched chain | | | | | | | |
| acyl-Coenzyme A dehydrogese, very | | 0.005 | 0.7043 | (−) | | | |
| long chain | | | | | | | |
| acyl-Coenzyme A oxidase 1, | | 8E−04 | 0.4926 | (−) | (+) | RCC | DC |
| palmitoyl | | | | | | | |
| adaptor-related protein complex AP- | 1.221326 | | | (+) | (+) | RCC | C |
| 3, sigma 1 subunit | | | | | | | |
| adducin 3 (gamma) | | 0.008 | 0.7735 | (−) | (+) | RCC | DC |
| adenine phosphoribosyl transferase | | 0.044 | 1.3581 | (+) | | | |
| adenylate cyclase 4 | 0.839219 | | | (−) | | | |
| adenylate kise 4 | 0.398031 | 8E−04 | 0.4203 | (−) | | | |
| adenylosuccite synthetase 2, non | 1.307874 | 0.01 | 1.4121 | (+) | | | |
| muscle | | | | | | | |
| adenylyl cyclase-associated CAP | 1.526675 | | | (+) | | | |
| protein homolog 1 (S. cerevisiae, S. pombe) | | | | | | | |
| ADP-ribosylation factor 1 | 1.301135 | | | (+) | | | |
| ADP-ribosyltransferase (D+ | 1.387701 | | | (+) | | | |
| AE binding protein 1 | | 0.035 | 1.4773 | (+) | | | |
| ajuba | | 0.004 | 1.2787 | (+) | | | |
| alcohol dehydrogese 4 (class II), pi | | 8E−04 | 0.5365 | (−) | (−) | RCC | C |
| polypeptide | | | | | | | |
| aldehyde dehydrogese family 1, | | 8E−04 | 1.6426 | (+) | | | |
| subfamily A2 | | | | | | | |
| aldo-keto reductase family 1, member | 1.868794 | 0.004 | 1.534 | (+) | | | |
| B8 ((Fgfrp) fibroblast growth factor | | | | | | | |
| regulated protein) | | | | | | | |
| aldo-keto reductase family 1, member | 0.403233 | | | (−) | | | |
| C18; expressed sequence AW146047 | | | | | | | |
| alkaline phosphatase 2, liver | 0.761972 | | | (−) | (−) | RCC | C |
| ALL1-fused gene from chromosome | 0.820461 | | | (−) | | | |
| 1q | | | | | | | |
| alpha-methylacyl-CoA racemase | 0.821375 | | | (−) | (+) | RCC | DC |
| amelogenin | | 0.043 | 1.7776 | (+) | | | |
| amiloride binding protein 1 (amine | 1.636321 | 8E−04 | 3.1046 | (+) | (+) | RCC | C |
| oxidase, copper-containing) | | | | | | | |
| amine N-sulfotransferase | 0.581682 | | | (−) | | | |
| aminoadipate-semialdehyde synthase/ | 0.505547 | 8E−04 | 0.4773 | (−) | | | |
| (Lorsdh) lysine oxoglutarate | | | | | | | |
| reductase, saccharopine dehydrogese | | | | | | | |
| AMP deamise 3 | | 0.006 | 1.2946 | (+) | | | |
| annexin A1 | | 8E−04 | 2.0545 | (+) | (+)/(???−) | RCC | conflict |
| annexin A2 | 3.930545 | 8E−04 | 2.6506 | (+) | (−)/(+) | RCC | conflict |
| annexin A3 | | 8E−04 | 2.1511 | (+) | | | |
| annexin A4 | | 0.002 | 1.4492 | (+) | (+) | RCC | C |
| annexin A5 | 1.762505 | 8E−04 | 1.7547 | (+) | | | |
| annexin A6 | 1.403621 | 0.038 | 1.4849 | (+) | | | |
| anterior gradient 2 (Xenopus laevis) | 0.74389 | | | (−) | | | |
| apolipoprotein B editing complex 1 | | 0.003 | 1.6053 | (+) | | | |
| apolipoprotein E | | 0.03 | 1.7135 | (+) | (−) | RCC | DC |
| apoptosis inhibitory protein 5 | | 0.046 | 1.2954 | (+) | | | |
| apurinic/apyrimidinic endonuclease | 1.513149 | | | (+) | | | |
| aquaporin 2 | 0.604517 | | | (−) | | | |
| arachidote 12-lipoxygese, pseudogene 2 | | 0.036 | 0.788 | (−) | | | |
| arachidote 5-lipoxygese activating | 1.299816 | | | (+) | (+) | RCC | C |
| protein | | | | | | | |
| arginine-rich, mutated in early stage | 1.304171 | | | (+) | | | |
| tumors | | | | | | | |
| argise type II | | 0.012 | 1.5597 | (+) | | | |
| Arpc2 | 1.6559 | 0.003 | 1.3245 | (+) | | | |
| ATP synthase, H+ transporting | 0.685294 | | | (−) | | | |
| mitochondrial F1 complex, beta | | | | | | | |
| subunit | | | | | | | |
| ATP synthase, H+ transporting, | 0.700665 | | | (−) | | | |
| mitochondrial F1 complex, alpha | | | | | | | |
| subunit, isoform 1 | | | | | | | |
| ATPase, +/K+ transporting, beta 1 | | 0.009 | 0.5031 | (−) | (+) | RCC | DC |
| polypeptide | | | | | | | |
| ATPase, H+ transporting, lysosomal | 0.773098 | | | (−) | | | |
| (vacuolar proton pump), alpha 70 kDa, | | | | | | | |
| isoform 1 | | | | | | | |
| ATPase, H+ transporting, V1 subunit | 0.836034 | | | (−) | | | |
| F; RIKEN cD 1110004G16 gene | | | | | | | |
| ATPase, H+/K+ transporting, alpha | 0.786786 | | | (−) | | | |
| polypeptide | | | | | | | |
| ATP-binding cassette, sub-family A | | 0.006 | 1.5416 | (+) | | | |
| (ABC1), member 7 | | | | | | | |
| ATP-binding cassette, sub-family D | 0.704394 | 8E−04 | 0.6847 | (−) | | | |
| (ALD), member 3 | | | | | | | |
| AU R binding protein/enoyl- | 0.727287 | 0.022 | 0.7063 | (−) | | | |
| coenzyme A hydratase | | | | | | | |
| avian reticuloendotheliosis viral (v- | | 0.006 | 1.3329 | (+) | | | |
| rel) oncogene related B | | | | | | | |
| AXL receptor tyrosine kise | 1.476698 | 0.002 | 1.5274 | (+) | | | |
| baculoviral IAP repeat-containing 1a | 1.479547 | 8E−04 | 1.6192 | (+) | | | |
| baculoviral IAP repeat-containing 2 | | 0.003 | 1.5062 | (+) | (+) | RCC | C |
| baculoviral IAP repeat-containing 3 | | 0.001 | 1.4791 | (+) | (+) | RCC | C |
| B-box and SPRY domain containing | | 0.002 | 1.3714 | (+) | | | |
| B-cell leukemia/lymphoma 2 related | 1.425202 | 0.002 | 1.9462 | (+) | | | |
| protein A1b | | | | | | | |
| BCL2-antagonist/killer 1 | | 0.04 | 1.2407 | (+) | | | |
| Bcl-2-related ovarian killer protein | | 8E−04 | 1.6566 | (+) | | | |
| benzodiazepine receptor, peripheral | | 0.003 | 1.5025 | (+) | | | |
| beta-2 microglobulin | | 8E−04 | 2.3092 | (+) | (+) | RCC | C |
| betaine-homocysteine | 0.463882 | | | (−) | (−) | RCC | C |
| methyltransferase | | | | | | | |
| biglycan | 1.526097 | 8E−04 | 1.9267 | (+) | | | |
| bisphosphate 3′-nucleotidase 1 | | 0.003 | 0.6085 | (−) | | | |
| Blu protein | 0.711446 | | | (−) | | | |
| bone marrow stromal cell antigen 1 | 1.303195 | 0.004 | 1.3219 | (+) | | | |
| bone morphogenetic protein receptor, | | 0.01 | 1.2873 | (+) | | | |
| type 1A | | | | | | | |
| brain protein 44-like | 0.660344 | | | (−) | (−) | RCC | C |
| branched chain aminotransferase 2, | 0.660946 | | | (−) | | | |
| mitochondrial | | | | | | | |
| branched chain ketoacid dehydrogese | 0.615398 | 8E−04 | 0.59 | (−) | (+) | RCC | DC |
| E1, alpha polypeptide | | | | | | | |
| breakpoint cluster region protein 1 | 1.639424 | | | (+) | | | |
| BRG1/brm-associated factor 53A | 1.348562 | 0.015 | 1.4078 | (+) | | | |
| Bromodomain and PHD finger | 0.78672 | | | (−) | | | |
| containing, 3 | | | | | | | |
| cadherin 3 | 1.349831 | 8E−04 | 1.4592 | (+) | | | |
| calbindin-28K | 0.327595 | 0.014 | 0.4917 | (−) | (−) | RCC | C |
| calbindin-D9K | 0.556398 | | | (−) | | | |
| calcium channel, voltage-dependent, | | 0.038 | 1.4187 | (+) | (+) | RCC | C |
| beta 3 subunit | | | | | | | |
| calpain 2 | | 0.001 | 1.2591 | (+) | | | |
| calpain, small subunit 1 | 0.584314 | | | (−) | (+) | RCC | DC |
| calponin 2 | 1.384116 | 8E−04 | 1.8214 | (+) | | | |
| calreticulin | 1.244306 | | | (+) | (−)/(+) | RCC | conflict |
| calsyntenin 1 | 0.761543 | | | (−) | (−) | RCC | C |
| capping protein beta 1 | 1.247283 | 0.023 | 1.4453 | (+) | | | |
| carbonic anhydrase 5a, mitochondrial | 0.793202 | | | (−) | | | |
| carboxylesterase 3 | 0.466372 | 0.008 | 0.5905 | (−) | | | |
| carboxypeptidase E | | 0.022 | 1.5977 | (+) | | | |
| carboxypeptidase X 1 (M14 family)/ | | 0.011 | 1.4083 | (+) | | | |
| metallocarboxypeptidase 1 | | | | | | | |
| cardiac responsive adriamycin protein | 1.578084 | | | (+) | | | |
| carnitine palmitoyltransferase 1, liver | 0.726551 | 0.002 | 0.5809 | (−) | (+) | RCC | DC |
| carnitine palmitoyltransferase 1, | 0.662861 | | | (−) | | | |
| muscle | | | | | | | |
| carnitine palmitoyltransferase 2 | 0.681572 | | | (−) | (−) | RCC | C |
| cartilage oligomeric matrix protein | 0.869318 | | | (−) | | | |
| casein kise 1, epsilon | | 0.028 | 1.3466 | (+) | | | |
| caspase 1 | 0.75804 | | | (−) | (+)/(−) | RCC | conflict |
| caspase 3, apoptosis related cysteine | | 0.004 | 1.3961 | (+) | | | |
| protease | | | | | | | |
| caspase 8 | 1.169654 | | | (+) | | | |
| cathepsin D | 1.996407 | | | (+) | (+) | RCC | C |
| cathepsin L | 1.206119 | | | (+) | | | |
| cathepsin S | 1.733231 | 8E−04 | 4.4853 | (+) | (+) | RCC | C |
| cathepsin Z | 1.23248 | | | (+) | | | |
| Cbp/p300-interacting transactivator | | 0.036 | 0.7565 | (−) | | | |
| with Glu/Asp-rich carboxy-termil | | | | | | | |
| domain 1 | | | | | | | |
| CCCTC-binding factor | 1.310333 | | | (+) | | | |
| CD24a antigen | 1.57732 | 8E−04 | 1.8903 | (+) | (+) | RCC | C |
| CD2-associated protein | 1.4548 | 8E−04 | 1.766 | (+) | (+) | RCC | C |
| CD38 antigen | 1.385877 | | | (+) | | | |
| CD48 antigen | | 8E−04 | 1.8446 | (+) | | | |
| CD52 antigen | | 0.0008; | 2.63371; | (+) | (+) | RCC | C |
| | | 0.0008 | 2.413666 | | | | |
| CD53 antigen | 1.453756 | 0.004 | 1.5299 | (+) | (+) | RCC | C |
| CD59a antigen | 0.783717 | | | (−) | (+) | RCC | DC |
| CD68 antigen | 1.767182 | 0.004 | 1.8367 | (+) | (+) | RCC | C |
| CD72 antigen | 1.295352 | 0.003 | 1.5366 | (+) | | | |
| CDC16 (cell division cycle 16 | 1.191802 | | | (+) | (+) | RCC | C |
| homolog (S. cerevisiae) | | | | | | | |
| CDC28 protein kise 1 | 1.370272 | | | (+) | (+) | RCC | C |
| CDK2 (cyclin-dependent kise 2)- | 1.291944 | | | (+) | | | |
| asscoaited protein 1 | | | | | | | |
| CEA-related cell adhesion molecule 1 | 0.670955 | 0.004 | 0.6695 | (−) | (+) | RCC | DC |
| CEA-related cell adhesion molecule 2 | 0.578039 | 0.014 | 0.6396 | (−) | | | |
| cell death-inducing D fragmentation | 0.662515 | | | (−) | | | |
| factor, alpha subunit-like effector B | | | | | | | |
| cell division cycle 2 homolog A (S. pombe) | 1.989204 | | | (+) | | | |
| cell division cycle 25 homolog A (S. cerevisiae) | 1.164267 | | | (+) | | | |
| cell division cycle 42 homolog (S. cerevisiae) | 1.309167 | 0.002 | 1.5138 | (+) | (+) | RCC | C |
| cellular nucleic acid binding protein | 1.26296 | | | (+) | (+) | RCC | C |
| centrin 2 | 0.850689 | | | (−) | | | |
| centrin 3 | | 0.032 | 1.2633 | (+) | | | |
| ceroid-lipofuscinosis, neurol 2 | 0.766857 | | | (−) | | | |
| chaperonin subunit 3 (gamma) | 1.631384 | | | (+) | | | |
| chemokine (C-C) receptor 2 | 1.379928 | 0.004 | 1.8554 | (+) | (+) | RCC | C |
| chemokine (C-C) receptor 5 | 1.37154 | | | (+) | | | |
| chemokine orphan receptor 1 | | 8E−04 | 1.7518 | (+) | | | |
| chitise 3-like 3 | 1.319784 | | | (+) | | | |
| chloride channel calcium activated 1 | | 0.02 | 1.325 | (+) | | | |
| chloride channel, nucleotide- | | 0.002 | 1.2654 | (+) | | | |
| sensitive, 1A | | | | | | | |
| chloride intracellular channel 1 | 2.425273 | 8E−04 | 1.9983 | (+) | (+) | RCC | C |
| chloride intracellular channel 4 | 1.319271 | 0.021 | 1.2476 | (+) | | | |
| (mitochondrial) | | | | | | | |
| cholinergic receptor, nicotinic, beta | | 0.009 | 1.3002 | (+) | | | |
| polypeptide 1 (muscle) | | | | | | | |
| citrate lyase beta like | 0.749572 | | | (−) | | | |
| clathrin, light polypeptide (Lca) | 1.279741 | | | (+) | | | |
| claudin 1 | 2.081215 | 0.001 | 1.5533 | (+) | (+) | RCC | C |
| claudin 4 | 1.584524 | 0.005 | 1.6885 | (+) | | | |
| claudin 7 | 1.628062 | 8E−04 | 1.4804 | (+) | | | |
| cleavage and polyadenylation specific | | 0.042 | 1.2755 | (+) | | | |
| factor 5, 25 kD subunit | | | | | | | |
| clusterin | 5.900022 | | | (+) | (?) | RCC | conflict |
| coagulation factor II (thrombin) | 1.422208 | 8E−04 | 1.3135 | (+) | | | |
| receptor-like 1 | | | | | | | |
| coagulation factor III | 2.368334 | 0.003 | 1.7004 | (+) | | | |
| coagulation factor XIII, beta subunit | 0.575972 | 8E−04 | 0.585 | (−) | | | |
| cofilin 1, non-muscle | 2.223096 | | | (+) | (+)/(−) | RCC | conflict |
| cold shock domain protein A | 1.93466 | 9E−04 | 1.3519 | (+) | (+) | RCC | C |
| colony stimulating factor 1 | 1.711817 | | | (+) | (+) | RCC | C |
| (macrophage) | | | | | | | |
| complement component 1, q | 1.61595 | 8E−04 | 2.7213 | (+) | (+) | RCC | C |
| subcomponent, alpha polypeptide | | | | | | | |
| complement component 1, q | | 8E−04 | 4.2321 | (+) | (+) | RCC | C |
| subcomponent, beta polypeptide | | | | | | | |
| complement component 1, q | | 8E−04 | 3.365 | (+) | | | |
| subcomponent, c polypeptide | | | | | | | |
| complement component 3 | 2.411628 | 8E−04 | 3.4754 | (+) | | | |
| complement component factor i | 1.508817 | | | (+) | (−) | RCC | DC |
| complement factor H related protein | | 0.0009; | 2.204364; | (+) | | | |
| 3A4/5G4 | | 0.0008 | 2.435881 | | | | |
| connective tissue growth factor | | 8E−04 | 1.6706 | (+) | (−) | RCC | DC |
| constitutive photomorphogenic | | 0.019 | 1.276 | (+) | | | |
| protein 1 (Arabidopsis) | | | | | | | |
| coproporphyrinogen oxidase | | 0.001 | 0.6349 | (−) | | | |
| cordon-bleu; ESTs, Moderately | 1.27206 | | | (+) | | | |
| similar to T00381 KIAA0633 protein | | | | | | | |
| (H. sapiens) | | | | | | | |
| core promoter element binding | 1.534502; | 0.0148; | 1.622871; | (+) | (+) | RCC | C |
| protein | 1.708834 | 0.0008 | 2.094609 | | | | |
| cornichon homolog (Drosophila) | 1.174252 | | | (+) | | | |
| coronin, actin binding protein 1B | 1.246811 | 0.022 | 1.4195 | (+) | (−) | RCC | DC |
| craniofacial development protein 1 | 1.358741 | 0.004 | 1.3837 | (+) | | | |
| creatine kise, brain | 0.625228 | | | (−) | | | |
| cryptochrome 2 (photolyase-like) | 0.75375 | | | (−) | | | |
| crystallin, alpha B | 1.724386 | | | (+) | (+) | RCC | C |
| crystallin, lamda 1 | 0.682398 | 9E−04 | 0.6419 | (−) | | | |
| crystallin, mu | 1.739818 | 8E−04 | 2.9709 | (+) | (−) | RCC | DC |
| cyclin E1 | 1.230927 | | | (+) | (+) | RCC | C |
| cyclin-dependent kise 4 | 1.709692 | | | (+) | | | |
| cyclin-dependent kise inhibitor 1A | 1.764317 | | | (+) | (+)/(+??) | RCC | conflict |
| (P21) | | | | | | | |
| cystatin B | 2.140696 | 8E−04 | 1.98 | (+) | | | |
| cystatin C | | 0.001 | 1.7744 | (+) | | | |
| cysteine rich protein 61 | 2.006582 | 0.005 | 1.8544 | (+) | (−) | RCC | DC |
| cytidine 5′-triphosphate synthase | 1.458773 | 0.006 | 1.3569 | (+) | | | |
| cytidine 5′-triphosphate synthase 2 | | 0.002 | 1.2751 | (+) | | | |
| cytochrome c oxidase, subunit VIc | 0.738692 | | | (−) | (+) | RCC | DC |
| cytochrome c oxidase, subunit VIIa 1 | 0.62639 | | | (−) | | | |
| cytochrome c oxidase, subunit VIIa 3 | 0.755682 | | | (−) | | | |
| cytochrome c oxidase, subunit VIIIa | | 0.003 | 0.772 | (−) | | | |
| cytochrome P450, 2a4 | 0.3663932; | 0.005; | 0.5020061; | (−) | | | |
| | 0.4095392 | 0.0089 | 0.4404707 | | | | |
| cytochrome P450, 2d9 | 0.4799 | 8E−04 | 0.5423 | (−) | | | |
| cytochrome P450, 2e1, ethanol | 0.63884 | | | (−) | | | |
| inducible | | | | | | | |
| cytochrome P450, 2j5 | 0.712681 | 0.016 | 0.7664 | (−) | | | |
| cytochrome P450, family 4, | | 0.014 | 1.5046 | (+) | | | |
| subfamily v, polypeptide 3/ | | | | | | | |
| expressed sequence AW111961 | | | | | | | |
| cytochrome P450, subfamily IV B, | | 0.002 | 0.4359 | (−) | | | |
| polypeptide 1 | | | | | | | |
| cytokine inducible SH2-containing | 2.296698 | 8E−04 | 2.0252 | (+) | | | |
| protein 3 | | | | | | | |
| D methyltransferase (cytosine-5) 1 | 1.45436 | | | (+) | | | |
| D methyltransferase 3B | 1.25679 | | | (+) | | | |
| D primase, p49 subunit | 1.356209 | | | (+) | | | |
| D segment, Chr 12, ERATO Doi 604, | | 0.025 | 1.3497 | (+) | | | |
| expressed | | | | | | | |
| D segment, Chr 17, ERATO Doi 441, | 1.385397 | 0.007 | 1.3747 | (+) | | | |
| expressed | | | | | | | |
| D segment, Chr 17, human D6S56E 2 | 1.274877 | | | (+) | | | |
| D segment, Chr 18, Wayne State | 0.790825 | 0.037 | 0.6998 | (−) | (−) | RCC | C |
| University 181, expressed | | | | | | | |
| D segment, Chr 8, Brigham & | 0.70845 | | | (−) | | | |
| Women's Genetics 1320 expressed | | | | | | | |
| damage specific D binding protein 1 | 1.248195 | | | (+) | | | |
| (127 kDa) | | | | | | | |
| D-amino acid oxidase | | 0.044 | 0.7267 | (−) | | | |
| D-dopachrome tautomerase | 0.687173 | | | (−) | (−) | RCC | C |
| DEAD/H (Asp-Glu-Ala-Asp/His) box | | 0.044 | 1.2423 | (+) | | | |
| polypeptide 50/nucleolar protein | | | | | | | |
| GU2 | | | | | | | |
| decorin | | 8E−04 | 1.6067 | (+) | (−) | RCC | DC |
| deiodise, iodothyronine, type I | 0.426139 | 0.004 | 0.5359 | (−) | | | |
| deltex 1 homolog (Drosophila) | 0.824274 | | | (−) | (−) | RCC | C |
| deoxyribonuclease I | 0.334306 | 8E−04 | 0.2485 | (−) | | | |
| diaphorase 1 (DH) | 1.27042 | 0.03 | 1.3708 | (+) | | | |
| dihydropyrimidise | 0.779607 | 0.002 | 0.7295 | (−) | (−) | RCC | C |
| dihydropyrimidise-like 3 | 1.24934 | | | (+) | (+) | RCC | C |
| dimethylarginine | | 0.002 | 1.4038 | (+) | | | |
| dimethylaminohydrolase 2 | | | | | | | |
| dipeptidase 1 (rel) | 0.543074 | 0.003 | 0.5863 | (−) | (−) | RCC | C |
| DJ (Hsp40) homolog, subfamily A, | 0.696704 | | | (−) | | | |
| member 1 | | | | | | | |
| DJ (Hsp40) homolog, subfamily B, | 0.805639 | | | (−) | | | |
| member 12 | | | | | | | |
| DJ (Hsp40) homolog, subfamily C, | | 0.022 | 1.2967 | (+) | | | |
| member 5 | | | | | | | |
| dolichyl-di-phosphooligosaccharide- | 1.354829 | | | (+) | | | |
| protein glycotransferase | | | | | | | |
| dopa decarboxylase | 0.755528 | | | (−) | (−) | RCC | C |
| double cortin and | 1.267038 | | | (+) | | | |
| calcium/calmodulin-dependent | | | | | | | |
| protein kise-like 1 | | | | | | | |
| downstream of tyrosine kise 1 | | 0.049 | 1.2419 | (+) | | | |
| DPH oxidase 4 | | 0.002 | 0.5556 | (−) | (?) | RCC | conflict |
| E26 avian leukemia oncogene 2, 3′ | 1.244631 | | | (+) | | | |
| domain | | | | | | | |
| E74-like factor 3 | 1.495613 | 8E−04 | 1.4218 | (+) | (+) | RCC | C |
| E74-like factor 4 (ets domain | 1.355901 | 0.009 | 1.2619 | (+) | | | |
| transcription factor) | | | | | | | |
| early development regulator 2 | | 0.004 | 1.4881 | (+) | | | |
| (homolog of polyhomeotic 2) | | | | | | | |
| ectonucleoside triphosphate | 0.79518 | | | (−) | | | |
| diphosphohydrolase 5 | | | | | | | |
| ectonucleotide | 0.578313 | 8E−04 | 0.6047 | (−) | (+) | RCC | DC |
| pyrophosphatase/phosphodiesterase 2 | | | | | | | |
| EGF-like module containing, mucin- | | 8E−04 | 2.0862 | (+) | | | |
| like, hormone receptor-like sequence 1 | | | | | | | |
| EGL nine homolog 1 (C. elegans) | 0.785405 | | | (−) | (+) | RCC | DC |
| elafin-like protein I | 0.289826 | | | (−) | | | |
| elastase 1, pancreatic | 0.579248 | | | (−) | | | |
| elongation of very long chain fatty | 1.690045 | 8E−04 | 2.7756 | (+) | | | |
| acids (FEN1/Elo2, SUR4/Elo3, | | | | | | | |
| yeast)-like 1 | | | | | | | |
| endonuclease G | 0.624758 | | | (−) | | | |
| endoplasmic reticulum protein 29 | | 0.028 | 1.384 | (+) | | | |
| endothelin 1 | 1.479734 | 8E−04 | 1.5711 | (+) | | | |
| enhancer of zeste homolog 2 | 1.357625 | | | (+) | | | |
| (Drosophila) | | | | | | | |
| enoyl Coenzyme A hydratase, short | 0.728878 | | | (−) | | | |
| chain, 1, mitochondrial | | | | | | | |
| epidermal growth factor | 0.115294 | 8E−04 | 0.1981 | (−) | (−) | RCC | C |
| epidermal growth factor-containing | | 0.002 | 1.4845 | (+) | | | |
| fibulin-like extracellular matrix | | | | | | | |
| protein 1 | | | | | | | |
| epidermal growth factor-containing | 1.736829 | 0.006 | 1.4624 | (+) | | | |
| fibulin-like extracellular matrix | | | | | | | |
| protein 2 | | | | | | | |
| epithelial membrane protein 3 | 1.838163 | 8E−04 | 1.4262 | (+) | (+) | RCC | C |
| erythrocyte protein band 4.1/Mus | | 0.017 | 0.7166 | (−) | (−) | RCC | C |
| musculus adult male tongue cD, | | | | | | | |
| RIKEN full-length enriched library, | | | | | | | |
| clone:2310065B16:erythrocyte | | | | | | | |
| protein band 4.1, full insert sequence | | | | | | | |
| erythrocyte protein band 4.1-like 1 | 0.82105 | | | (−) | | | |
| erythroid differentiation regulator | 1.550627 | | | (+) | | | |
| EST AI181838 | 0.72178 | | | (−) | | | |
| estrogen related receptor, alpha | 0.732545 | | | (−) | | | |
| ESTs | 0.735494 | 0.001 | 0.7011 | (−) | | | |
| ESTs | 0.631426 | 0.035 | 0.697 | (−) | | | |
| ESTs | 1.306482 | | | (+) | | | |
| ESTs | 0.772863 | | | (−) | | | |
| ESTs | 0.809355 | | | (−) | | | |
| ESTs | 1.345273 | | | (+) | | | |
| ESTs | 0.876828 | | | (−) | | | |
| ESTs | 1.357738 | | | (+) | | | |
| ESTs | 0.685626 | | | (−) | | | |
| ESTs | 0.804817 | | | (−) | | | |
| ESTs | 1.327383 | | | (+) | | | |
| ESTs | 0.498174 | | | (−) | | | |
| ESTs | 1.266278 | | | (+) | | | |
| ESTs | 0.755656 | | | (−) | | | |
| ESTs | 0.852094 | | | (−) | | | |
| ESTs | 0.844027 | | | (−) | | | |
| ESTs | 0.835016 | | | (−) | | | |
| ESTs | 1.316725 | | | (+) | | | |
| ESTs | 0.739721 | | | (−) | | | |
| ESTs | 0.733193 | | | (−) | | | |
| ESTs | 0.797542 | | | (−) | | | |
| ESTs | 0.855551 | | | (−) | | | |
| ESTs | 1.258533 | | | (+) | | | |
| ESTs | 0.810287 | | | (−) | | | |
| ESTs | 0.813422 | | | (−) | | | |
| ESTs | 0.788013 | | | (−) | | | |
| ESTs | 1.346671 | | | (+) | | | |
| ESTs | 1.30085 | | | (+) | | | |
| ESTs | | 0.015 | 1.2779 | (+) | | | |
| ESTs | | 0.005 | 1.301 | (+) | | | |
| ESTs | | 0.003 | 1.5954 | (+) | | | |
| ESTs | | 8E−04 | 1.7006 | (+) | | | |
| ESTs | | 0.047 | 0.8025 | (−) | | | |
| ESTs | | 8E−04 | 1.582 | (+) | | | |
| ESTs | | 0.006 | 1.3173 | (+) | | | |
| ESTs | | 0.036 | 0.7972 | (−) | | | |
| ESTs | | 0.009 | 0.7379 | (−) | | | |
| ESTs | | 0.009 | 1.3453 | (+) | | | |
| ESTs | | 0.021 | 0.7619 | (−) | | | |
| ESTs | | 0.004 | 0.8135 | (−) | | | |
| ESTs | | 0.014 | 0.6346 | (−) | | | |
| ESTs | | 0.014 | 0.6812 | (−) | | | |
| ESTs-pending | 1.272639 | | | (+) | | | |
| ESTs, Highly similar to prefoldin 4 | 1.245303 | | | (+) | (+) | RCC | C |
| (Homo sapiens) (H. sapiens) | | | | | | | |
| ESTs, Highly similar to organic | 0.728299 | | | (−) | | | |
| cation transporter-like protein 2 | | | | | | | |
| (M. musculus) | | | | | | | |
| ESTs, Highly similar to T00268 | 0.736573 | | | (−) | | | |
| hypothetical protein KIAA0597 | | | | | | | |
| (H. sapiens) | | | | | | | |
| ESTs, Moderately similar to SEC7 | | 0.005 | 0.6194 | (−) | | | |
| homolog (Homo sapiens) (H. sapiens) | | | | | | | |
| ESTs, Moderately similar to S12207 | 0.560434 | 0.004 | 0.6775 | (−) | | | |
| hypothetical protein (M. musculus) | | | | | | | |
| ESTs, Moderately similar to T08673 | 0.733259 | 0.012 | 0.6844 | (−) | (−) | RCC | C |
| hypothetical protein | | | | | | | |
| DKFZp564C0222.1 (H. sapiens) | | | | | | | |
| ESTs, Moderately similar to T46312 | | 0.005 | 1.4121 | (+) | | | |
| hypothetical protein | | | | | | | |
| DKFZp434J1111.1 (H. sapiens) | | | | | | | |
| ESTs, Weakly similar to brain- | 0.743618 | | | (−) | | | |
| specific angiogenesis inhibitor 1- | | | | | | | |
| associated protein 2 (Mus musculus) | | | | | | | |
| (M. musculus) | | | | | | | |
| ESTs, Weakly similar to limb | 1.18303 | | | (+) | | | |
| expression 1 homolog (chicken) (Mus | | | | | | | |
| musculus) (M. musculus) | | | | | | | |
| ESTs, Weakly similar to simple | | 8E−04 | 1.2461 | (+) | | | |
| repeat sequence-containing transcript | | | | | | | |
| (Mus musculus) (M. musculus) | | | | | | | |
| ESTs, Weakly similar to 2022314A | | 0.01 | 1.3354 | (+) | | | |
| granule cell marker protein | | | | | | | |
| (M. musculus) | | | | | | | |
| ESTs, Weakly similar to ADT1 | 0.834522 | | | (−) | | | |
| MOUSE ADP, ATP CARRIER | | | | | | | |
| PROTEIN, HEART/SKELETAL | | | | | | | |
| MUSCLE ISOFORM T1 | | | | | | | |
| (M. musculus) | | | | | | | |
| ESTs, Weakly similar to ADT1 | 0.78616 | | | (−) | | | |
| MOUSE ADP, ATP CARRIER | | | | | | | |
| PROTEIN, HEART/SKELETAL | | | | | | | |
| MUSCLE ISOFORM T1 | | | | | | | |
| (M. musculus) | | | | | | | |
| ESTs, Weakly similar to AF182426 1 | 0.651341 | 8E−04 | 0.6067 | (−) | | | |
| arylacetamide deacetylase | | | | | | | |
| (R. norvegicus) | | | | | | | |
| ESTs, Weakly similar to B Chain B, | | 0.001 | 1.2499 | (+) | | | |
| Crystal Structure Of Murine Soluble | | | | | | | |
| Epoxide Hydrolase Complexed With | | | | | | | |
| Cdu Inhibitor (M. musculus) | | | | | | | |
| ESTs, Weakly similar to DRR1 | 0.712178 | 0.015 | 0.7241 | (−) | | | |
| (H. sapiens) | | | | | | | |
| ESTs, Weakly similar to JC7182 +- | 0.840269 | | | (−) | | | |
| dependent vitamin C (H. sapiens) | | | | | | | |
| ESTs, Weakly similar to JE0096 | | 0.025 | 1.3969 | (+) | | | |
| myocilin - mouse (M. musculus) | | | | | | | |
| ESTs, Weakly similar to MAJOR | | 0.03 | 0.8009 | (−) | | | |
| URIRY PROTEIN 4 PRECURSOR | | | | | | | |
| (M. musculus) | | | | | | | |
| ESTs, Weakly similar to S26689 | 0.841829 | | | (−) | | | |
| hypothetical protein hc1 - mouse | | | | | | | |
| (M. musculus) | | | | | | | |
| ESTs, Weakly similar to S65210 | 0.793096 | | | (−) | | | |
| hypothetical protein YPL191c - yeast | | | | | | | |
| (Saccharomyces cerevisiae) | | | | | | | |
| (S. cerevisiae) | | | | | | | |
| ESTs, Weakly similar to T29029 | 1.20938 | | | (+) | | | |
| hypothetical protein F53G12.5 - | | | | | | | |
| Caenorhabditis elegans (C. elegans) | | | | | | | |
| ESTs, Weakly similar to TS13 | | 0.008 | 1.2414 | (+) | | | |
| MOUSE TESTIS-SPECIFIC | | | | | | | |
| PROTEIN PBS13 (M. musculus) | | | | | | | |
| ESTs, Weakly similar to | 0.70538 | 0.009 | 0.6835 | (−) | | | |
| TYROSINE-PROTEIN KISE JAK3 | | | | | | | |
| (M. musculus) | | | | | | | |
| ESTs, Weakly similar to | 0.793884 | | | (−) | | | |
| TYROSINE-PROTEIN KISE JAK3 | | | | | | | |
| (M. musculus) | | | | | | | |
| ESTs, Weakly similar to | 1.330213 | | | (+) | | | |
| TYROSINE-PROTEIN KISE JAK3 | | | | | | | |
| (M. musculus) | | | | | | | |
| ESTs, Weakly similar to | 0.870445 | | | (−) | | | |
| YAE6_YEAST HYPOTHETICAL | | | | | | | |
| 13.4 KD PROTEIN IN ACS1-GCV3 | | | | | | | |
| INTERGENIC REGION | | | | | | | |
| (S. cerevisiae) | | | | | | | |
| ESTs, Weakly similar to | 2.10875 | 0.004 | 1.8813 | (+) | | | |
| YMP2_CAEEL HYPOTHETICAL | | | | | | | |
| 30.3 KD PROTEIN B0361.2 IN | | | | | | | |
| CHROMOSOME III (C. elegans) | | | | | | | |
| eukaryotic translation initiation factor | | 0.005 | 1.294 | (+) | | | |
| 2A | | | | | | | |
| eukaryotic translation initiation factor 3 | 1.274304 | | | (+) | | | |
| eukaryotic translation initiation factor | 1.340807 | | | (+) | (+) | RCC | C |
| 3, subunit 4 (delta, 44 kDa) | | | | | | | |
| eukaryotic translation initiation factor | 1.219128 | | | (+) | (+) | RCC | C |
| 4, gamma 2 | | | | | | | |
| eukaryotic translation initiation factor | 1.342776 | 8E−04 | 1.506 | (+) | (+) | RCC | C |
| 4A1 | | | | | | | |
| eukaryotic translation initiation factor | 0.840329 | | | (−) | (+) | RCC | DC |
| 4A2 | | | | | | | |
| eukaryotic translation initiation factor | 1.627646 | 0.009 | 1.5179 | (+) | | | |
| 4E binding protein 1 | | | | | | | |
| eukaryotic translation initiation factor | 1.571166 | | | (+) | | | |
| 5A | | | | | | | |
| E-vasodilator stimulated | | 0.044 | 1.316 | (+) | (+) | RCC | C |
| phosphoprotein | | | | | | | |
| exportin 1, CRM1 homolog (yeast) | 1.4997 | | | (+) | (+) | RCC | C |
| expressed in non-metastatic cells 2, | 1.329781 | | | (+) | (+) | RCC | C |
| protein (NM23B) (nucleoside | | | | | | | |
| diphosphate kise) | | | | | | | |
| expressed sequence AA408783 | | 0.005 | 1.5176 | (+) | (+) | RCC | C |
| expressed sequence AA589392 | 1.21524 | | | (+) | | | |
| expressed sequence AA672638 | 0.777122 | | | (−) | | | |
| expressed sequence AI117581 | 0.892163 | | | (−) | | | |
| expressed sequence AI118577 | 0.739771 | 0.021 | 0.7424 | (−) | | | |
| expressed sequence AI132189 | 0.706946 | | | (−) | | | |
| expressed sequence AI132321 | 1.342358 | 8E−04 | 2.4148 | (+) | | | |
| expressed sequence AI159688 | 0.465349 | 0.008 | 0.5963 | (−) | | | |
| expressed sequence AI182282 | 0.39936 | | | (−) | | | |
| expressed sequence AI182284 | 0.610678 | 8E−04 | 0.5623 | (−) | | | |
| expressed sequence AI194696 | | 8E−04 | 2.0538 | (+) | | | |
| expressed sequence AI265322 | 0.786084 | | | (−) | | | |
| expressed sequence AI314027 | | 0.003 | 1.3621 | (+) | | | |
| expressed sequence AI315037 | 0.873898 | | | (−) | | | |
| expressed sequence AI316828 | | 0.002 | 1.29 | (+) | | | |
| expressed sequence AI413331 | | 0.022 | 1.2847 | (+) | | | |
| expressed sequence AI447451 | | 8E−04 | 1.3615 | (+) | | | |
| expressed sequence AI448003 | | 0.014 | 1.3551 | (+) | | | |
| expressed sequence AI449309 | | 0.02 | 1.3528 | (+) | | | |
| expressed sequence AI450991 | 1.170481 | | | (+) | | | |
| expressed sequence AI461788 | 1.143531 | | | (+) | | | |
| expressed sequence AI465301 | 0.826408 | | | (−) | | | |
| expressed sequence AI480660 | 0.819368 | | | (−) | | | |
| expressed sequence AI504062 | 1.236201 | 0.008 | 1.3717 | (+) | | | |
| expressed sequence AI507121 | 0.674087 | | | (−) | | | |
| expressed sequence AI528491 | 0.799738 | | | (−) | | | |
| expressed sequence AI553555 | 0.731077 | | | (−) | | | |
| expressed sequence AI558103 | 0.804878 | | | (−) | | | |
| expressed sequence AI586180 | 1.401176 | 9E−04 | 1.3448 | (+) | | | |
| expressed sequence AI593249 | 0.503496 | 0.002 | 0.7107 | (−) | | | |
| expressed sequence AI593524 | | 0.017 | 0.7462 | (−) | | | |
| expressed sequence AI604920 | | 8E−04 | 1.433 | (+) | | | |
| expressed sequence AI607846 | 1.297307 | 0.003 | 1.5455 | (+) | | | |
| expressed sequence AI646725 | | 0.046 | 0.7871 | (−) | | | |
| expressed sequence AI661919 | | 0.006 | 0.8064 | (−) | | | |
| expressed sequence AI835705 | 0.63364 | | | (−) | | | |
| expressed sequence AI836219 | 0.779958 | | | (−) | | | |
| expressed sequence AI838057 | 0.711501 | | | (−) | | | |
| expressed sequence AI843960 | | 0.008 | 1.2221 | (+) | | | |
| expressed sequence AI844685 | 0.703625 | | | (−) | | | |
| expressed sequence AI844876 | | 0.003 | 0.7703 | (−) | | | |
| expressed sequence AI848669 | 0.925143 | | | (−) | | | |
| expressed sequence AI852479 | 0.776527 | | | (−) | | | |
| expressed sequence AI875199 | 0.768454 | | | (−) | | | |
| expressed sequence AI875557 | 0.724579 | | | (−) | | | |
| expressed sequence AI957255 | 0.692752 | | | (−) | | | |
| expressed sequence AI987692 | | 0.019 | 1.2573 | (+) | | | |
| expressed sequence AL022757 | 1.770321 | | | (+) | | | |
| expressed sequence AU015645 | 0.679211 | 0.011 | 0.6889 | (−) | | | |
| expressed sequence AU018056 | 0.813815 | | | (−) | | | |
| expressed sequence AU019833 | | 0.047 | 1.2608 | (+) | | | |
| expressed sequence AU042434 | | 0.018 | 1.3037 | (+) | | | |
| expressed sequence AV046379 | 0.82172 | 0.027 | 0.7278 | (−) | | | |
| expressed sequence AW045860 | | 0.038 | 0.8088 | (−) | | | |
| expressed sequence AW047581 | | 0.031 | 1.3428 | (+) | | | |
| expressed sequence AW124722 | 0.803501 | | | (−) | | | |
| expressed sequence AW261723 | 0.668321 | 0.001 | 0.6447 | (−) | | | |
| expressed sequence AW413625 | 1.269501 | | | (+) | | | |
| expressed sequence AW488255 | 0.877549 | | | (−) | | | |
| expressed sequence AW493404 | | 0.009 | 1.2209 | (+) | | | |
| expressed sequence AW541137 | | 0.044 | 1.32 | (+) | | | |
| expressed sequence AW552393 | 0.890969 | | | (−) | | | |
| expressed sequence AW743884 | | 8E−04 | 2.0791 | (+) | | | |
| expressed sequence BB120430 | 1.229521 | | | (+) | | | |
| expressed sequence C79732 | 0.742988 | | | (−) | | | |
| expressed sequence C80913 | | 0.029 | 1.1929 | (+) | | | |
| expressed sequence C81457 | | 0.011 | 0.5924 | (−) | | | |
| expressed sequence C85317 | | 0.007 | 1.3134 | (+) | | | |
| expressed sequence C85457 | 0.841033 | | | (−) | | | |
| expressed sequence C86169 | 0.771679 | | | (−) | | | |
| expressed sequence C86302 | 1.186345 | | | (+) | | | |
| expressed sequence C87222 | 1.388445 | 0.005 | 1.3635 | (+) | | | |
| expressed sequence R75232 | 1.903157 | | | (+) | | | |
| Fas apoptotic inhibitory molecule | | 0.001 | 1.3142 | (+) | | | |
| fatty acid synthase | 0.487362 | | | (−) | | | |
| f-box only protein 3 | 0.895328 | | | (−) | | | |
| Fc receptor, IgE, high affinity I, | 1.669993 | 8E−04 | 2.1723 | (+) | (+) | RCC | C |
| gamma polypeptide | | | | | | | |
| Fc receptor, IgG, low affinity III | 1.528608 | 9E−04 | 1.6917 | (+) | (+) | RCC | C |
| feline sarcoma oncogene | 1.220261 | | | (+) | (+) | RCC | C |
| fibrillarin | 1.408148 | | | (+) | (+) | RCC | C |
| fibrillin 1 | 1.603484 | 0.009 | 1.583 | (+) | | | |
| fibulin 5 | 0.547159 | | | (−) | | | |
| FK506 binding protein 10 (65 kDa) | 1.569148 | | | (+) | | | |
| FK506 binding protein 12-rapamycin | 0.6659 | 0.014 | 0.7232 | (−) | (+) | RCC | DC |
| associated protein 1 | | | | | | | |
| FK506 binding protein 1a (12 kDa) | 1.631333 | | | (+) | | | |
| FK506 binding protein 5 (51 kDa) | | 8E−04 | 0.5428 | (−) | | | |
| FK506 binding protein 9 | 1.218167 | | | (+) | | | |
| flap structure specific endonuclease 1 | 1.324505 | | | (+) | (+) | RCC | C |
| flavin containing monooxygese 1 | 0.624819 | | | (−) | (−) | RCC | C |
| flotillin 1 | 1.818412 | | | (+) | | | |
| flotillin 2 | 1.424145 | | | (+) | | | |
| folate receptor 1 (adult) | 0.654384 | 0.009 | 0.7132 | (−) | (−)/(+) | RCC | conflict |
| forkhead box M1 | 1.42683 | | | (+) | | | |
| four and a half LIM domains 1 | | 0.007 | 0.736 | (−) | (+) | RCC | DC |
| fragile histidine triad gene | 1.305838 | | | (+) | (−) | RCC | DC |
| fumarylacetoacetate hydrolase | 0.554798 | 8E−04 | 0.5524 | (−) | (−) | RCC | C |
| FXYD domain-containing ion | | 0.008 | 0.6338 | (−) | (−) | RCC | C |
| transport regulator 2 | | | | | | | |
| FXYD domain-containing ion | 1.873781 | 8E−04 | 1.5927 | (+) | | | |
| transport regulator 5 | | | | | | | |
| G protein-coupled receptor kise 7 | 0.743286 | | | (−) | (+) | RCC | DC |
| G1 to phase transition 1 | 1.490601 | | | (+) | | | |
| gamma-glutamyl hydrolase | | 0.013 | 1.2696 | (+) | (+)/(−) | RCC | conflict |
| gamma-glutamyl transpeptidase | 0.562559 | 8E−04 | 0.5141 | (−) | | | |
| ganglioside-induced differentiation- | | 0.029 | 1.262 | (+) | | | |
| associated-protein 3 | | | | | | | |
| gap junction membrane channel | | 0.034 | 0.6818 | (−) | (+) | RCC | DC |
| protein beta 2 | | | | | | | |
| glucose regulated protein, 58 kDa | 1.334846 | | | (+) | (+) | RCC | C |
| glucose-6-phosphatase, catalytic | 0.331086 | 8E−04 | 0.3315 | (−) | | | |
| glucose-6-phosphatase, transport | 0.504687 | | | (−) | | | |
| protein 1 | | | | | | | |
| glutamine synthetase | 0.506746 | 8E−04 | 0.3378 | (−) | | | |
| glutaryl-Coenzyme A dehydrogese | 0.620166 | 8E−04 | 0.5593 | (−) | | | |
| glutathione peroxidase 1 | 1.376036 | | | (+) | (+) | RCC | C |
| glutathione S-transferase, alpha 2 | | 0.01 | 0.6945 | (−) | (+)/(−) | RCC | conflict |
| (Yc2) | | | | | | | |
| glutathione S-transferase, alpha 4 | | 0.028 | 0.6627 | (−) | | | |
| glutathione S-transferase, mu 6 | 1.475521 | | | (+) | | | |
| glutathione S-transferase, pi 1 | 1.385566 | | | (+) | | | |
| glutathione S-transferase, theta 2 | 0.636317 | | | (−) | (−) | RCC | C |
| glutathione transferase zeta 1 | 0.634449 | | | (−) | | | |
| (maleylacetoacetate isomerase) | | | | | | | |
| glycerol kise | 0.520913 | 0.002 | 0.5752 | (−) | (−) | RCC | C |
| glycerol phosphate dehydrogese 1, | | 0.004 | 0.6803 | (−) | | | |
| mitochondrial | | | | | | | |
| glycerol-3-phosphate acyltransferase, | 0.66301 | 0.002 | 0.7084 | (−) | | | |
| mitochondrial | | | | | | | |
| glycine amidinotransferase (L- | 0.543395 | 0.003 | 0.6865 | (−) | (−) | RCC | C |
| arginine:glycine amidinotransferase) | | | | | | | |
| glycine N-methyltransferase | 0.580827 | | | (−) | | | |
| glycoprotein 49 A | 1.8182 | 0.002 | 1.8947 | (+) | | | |
| glycoprotein 49 B | 1.831723 | 0.013 | 1.6056 | (+) | | | |
| glypican 3 | | 8E−04 | 2.3509 | (+) | (−) | RCC | DC |
| golgi autoantigen, golgin subfamily a, 4 | 0.744408 | | | (−) | | | |
| golgi reassembly stacking protein 2 | 1.172165 | 0.007 | 1.291 | (+) | (+) | RCC | C |
| GPI-anchored membrane protein 1 | 1.309942 | | | (+) | (+) | RCC | C |
| granulin | 1.290686 | | | (+) | (+) | RCC | C |
| G-rich RNA sequence binding factor | | 0.028 | 0.7285 | (−) | (+) | RCC | DC |
| 1 (D5Wsu31e) D segment, Chr 5, | | | | | | | |
| Wayne State University 31, expressed | | | | | | | |
| group specific component | 1.498652 | | | (+) | (−) | RCC | DC |
| growth arrest and D-damage- | 1.493038 | 0.002 | 1.6622 | (+) | | | |
| inducible 45 alpha | | | | | | | |
| growth arrest and D-damage- | | 0.001 | 0.4592 | (−) | (+) | RCC | DC |
| inducible 45 gamma | | | | | | | |
| growth arrest specific 2 | 0.632398 | 8E−04 | 0.6609 | (−) | (−) | RCC | C |
| growth differentiation factor 15 | 1.635441 | 0.045 | 1.5152 | (+) | (+) | RCC | C |
| growth differentiation factor 8 | | 0.001 | 1.3728 | (+) | | | |
| growth factor receptor bound protein 7 | 0.798278 | | | (−) | (−) | RCC | C |
| guanine nucleotide binding protein (G | | 0.022 | 1.316 | (+) | | | |
| protein), gamma 2 subunit | | | | | | | |
| guanine nucleotide binding protein (G | 0.497877 | 0.001 | 0.5933 | (−) | | | |
| protein), gamma 5 subunit | | | | | | | |
| guanine nucleotide binding protein, | 1.428688 | 0.005 | 1.6772 | (+) | (+) | RCC | C |
| alpha inhibiting 2 | | | | | | | |
| guanine nucleotide binding protein, | 1.942687 | 0.001 | 1.4495 | (+) | (+) | RCC | C |
| beta 2, related sequence 1 | | | | | | | |
| guanosine diphosphate (GDP) | 1.194521 | | | (+) | | | |
| dissociation inhibitor 3 | | | | | | | |
| guanosine monophosphate reductase | 1.409698 | 0.042 | 1.4131 | (+) | | | |
| guanylate nucleotide binding protein 2 | | 8E−04 | 1.83 | (+) | (+) | RCC | C |
| H2A histone family, member Z | 1.937214 | 0.025 | 1.5002 | (+) | (+) | RCC | C |
| H2B histone family, member S | 0.757011 | | | (−) | | | |
| Harvey rat sarcoma oncogene, | 1.512845 | | | (+) | | | |
| subgroup R | | | | | | | |
| heat shock 70 kDa protein 4 | 1.296849; | | | (+) | | | |
| | 1.316802 | | | | | | |
| heat shock protein 1 (chaperonin)/ | | 9E−04 | 0.6689 | (−) | (+) | RCC | DC |
| heat shock protein, 60 kDa | | | | | | | |
| heat shock protein, 105 kDa | | 0.015 | 0.729 | (−) | (+) | RCC | DC |
| heat shock protein, 86 kDa 1 | 1.645544 | | | (+) | (?) | RCC | conflict |
| heat-responsive protein 12 | 0.647694 | | | (−) | (−) | RCC | C |
| hematological and neurological | 1.563803 | | | (+) | (+) | RCC | C |
| expressed sequence 1 | | | | | | | |
| heme oxygese (decycling) 1 | 1.922685 | | | (+) | | | |
| hemochromatosis | | 0.001 | 1.2616 | (+) | | | |
| hemopoietic cell phosphatase | 1.582381 | 9E−04 | 1.5358 | (+) | (+) | RCC | C |
| heparan sulfate 2-O-sulfotransferase 1 | 1.173811 | | | (+) | | | |
| heparin binding epidermal growth | 1.358949 | | | (+) | | | |
| factor-like growth factor | | | | | | | |
| hepatic nuclear factor 4 | | 8E−04 | 0.6498 | (−) | | | |
| hepatoma-derived growth factor | 1.180861 | | | (+) | | | |
| hepsin | 0.761344 | 0.036 | 0.7761 | (−) | (−) | RCC | C |
| heterogeneous nuclear | 2.419538 | 8E−04 | 1.8593 | (+) | (+) | RCC | C |
| ribonucleoprotein A1 | | | | | | | |
| hexokise 1 | 0.766611 | | | (−) | (+) | RCC | DC |
| high mobility group AT-hook 1 | 2.462143 | | | (+) | | | |
| high mobility group box 3 | 1.355483 | 0.002 | 1.564 | (+) | (+) | RCC | C |
| high mobility group nucleosomal | 1.760107 | 0.018 | 1.2532 | (+) | (+) | RCC | C |
| binding domain 2 | | | | | | | |
| histidyl tR synthetase | 0.708007 | | | (−) | (+) | RCC | DC |
| histocompatibility 2, class II antigen | | 8E−04 | 4.0415 | (+) | | | |
| A, alpha | | | | | | | |
| histocompatibility 2, class II antigen | | 8E−04 | 2.9829 | (+) | | | |
| E beta | | | | | | | |
| histocompatibility 2, class II, locus | | 0.002 | 1.7963 | (+) | | | |
| DMa | | | | | | | |
| Histocompatibility 2, D region locus 1 | 1.483204 | 8E−04 | 1.9955 | (+) | | | |
| histocompatibility 2, Q region locus 7 | | 0.005 | 1.6855 | (+) | | | |
| histone 2, H2aa1/(Hist2) histone | | 0.026 | 0.7303 | (−) | | | |
| gene complex 2 | | | | | | | |
| histone deacetylase 1 | | 0.012 | 1.4367 | (+) | | | |
| homeo box B7 | 1.189729 | | | (+) | | | |
| homocysteine-inducible, endoplasmic | 0.52813 | 8E−04 | 0.4351 | (−) | | | |
| reticulum stress-inducible, ubiquitin- | | | | | | | |
| like domain member 1 | | | | | | | |
| Hoxc8 | 1.638671 | | | (+) | | | |
| Hprt | 1.377124 | | | (+) | | | |
| hyaluron mediated motility receptor | 1.236898 | | | (+) | | | |
| (RHAMM) | | | | | | | |
| hyaluronic acid binding protein 2 | | 0.044 | 0.7814 | (−) | | | |
| hydroxysteroid 17-beta dehydrogese 7 | | 0.014 | 0.7563 | (−) | | | |
| hydroxysteroid dehydrogese-1, | 0.537309 | | | (−) | | | |
| delta<5>-3-beta | | | | | | | |
| hydroxysteroid dehydrogese-3, | 0.57926 | | | (−) | | | |
| delta<5>-3-beta | | | | | | | |
| hypothetical protein, I54 | 0.496484 | 9E−04 | 0.5491 | (−) | | | |
| hypothetical protein, MGC: 6957 | | 0.024 | 1.3597 | (+) | | | |
| hypothetical protein, MNCb-5210 | | 0.004 | 1.5476 | (+) | | | |
| Ia-associated invariant chain | | 8E−04 | 4.38 | (+) | (+) | RCC | C |
| immunoglobulin superfamily, | 1.150677 | | | (+) | | | |
| member 8 | | | | | | | |
| importin 11 (RIKEN cD 2510001A17 | 1.293414 | | | (+) | | | |
| gene) | | | | | | | |
| inhibin beta-B | 1.257506 | | | (+) | (+) | RCC | C |
| inhibitor of D binding 2 | | 8E−04 | 1.4816 | (+) | (+) | RCC | C |
| inosine 5′-phosphate dehydrogese 2 | 1.550038 | | | (+) | | | |
| inositol polyphosphate-5- | 0.700199 | 0.037 | 0.7627 | (−) | | | |
| phosphatase, 75 kDa | | | | | | | |
| insulin-like growth factor binding | 0.682742 | | | (−) | (+) | RCC | DC |
| protein 1 | | | | | | | |
| insulin-like growth factor binding | 0.558403 | | | (−) | (+) | RCC | DC |
| protein 3 | | | | | | | |
| insulin-like growth factor binding | 0.574239 | | | (−) | | | |
| protein 4 | | | | | | | |
| insulin-like growth factor binding | 0.738802 | | | (−) | | | |
| protein, acid labile subunit | | | | | | | |
| integrin alpha 6 | | 0.03 | 1.4584 | (+) | (+) | RCC | C |
| integrin alpha M | 1.291467 | | | (+) | (+) | RCC | C |
| integrin beta 1 (fibronectin receptor | | 8E−04 | 1.5674 | (+) | (+) | RCC | C |
| beta) | | | | | | | |
| integrin-associated protein | | 0.019 | 1.4362 | (+) | (+)/?) | RCC | conflict |
| intercellular adhesion molecule | 1.556701 | 0.021 | 1.5598 | (+) | (+) | RCC | C |
| interferon activated gene 204 | | 0.0014; | 1.686958; | (+) | | | |
| | | 0.0038 | 1.556905 | | | | |
| interferon gamma receptor | | 0.006 | 1.497 | (+) | (+) | RCC | C |
| interferon inducible protein 1 | 0.707584 | | | (−) | | | |
| interferon-induced protein with | 1.847808 | | | (+) | | | |
| tetratricopeptide repeats 3 | | | | | | | |
| intergral membrane protein 1 | 1.321916 | | | (+) | | | |
| interleukin 1 beta | 1.536653 | | | (+) | (?) | RCC | conflict |
| interleukin 1 receptor, type I | 1.304397 | | | (+) | | | |
| interleukin 11 receptor, alpha chain 1 | 0.723197 | | | (−) | | | |
| isocitrate dehydrogese 2 (DP+), | 0.756124 | 0.003 | 0.7726 | (−) | | | |
| mitochondrial | | | | | | | |
| isovaleryl coenzyme A dehydrogese | 0.6145993; | 0.004 | 0.6321 | (−) | | | |
| | 0.5060046 | | | | | | |
| J domain protein 1 | 0.583849 | 0.005 | 0.5726 | (−) | | | |
| junction plakoglobin | 0.554028 | | | (−) | (−) | RCC | C |
| kallikrein 26 | 0.573494 | 0.029 | 0.6276 | (−) | | | |
| kallikrein 6 | 0.625692 | 8E−04 | 0.5089 | (−) | (+) | RCC | DC |
| karyopherin (importin) alpha 2 | 1.591718 | | | (+) | (+) | RCC | C |
| karyopherin (importin) beta 3 | 1.334861 | | | (+) | | | |
| keratin complex 1, acidic, gene 19 | | 0.041 | 1.5647 | (+) | (+) | RCC | C |
| keratin complex 2, basic, gene 8 | 3.335629 | 8E−04 | 2.1229 | (+) | (+) | RCC | C |
| ketohexokise | 0.408655 | 0.018 | 0.629 | (−) | (−) | RCC | C |
| kidney-derived aspartic protease-like | 0.351128 | 8E−04 | 0.4507 | (−) | | | |
| protein | | | | | | | |
| kinectin 1 | | 0.003 | 1.3275 | (+) | | | |
| kinesin family member 1B (expressed | 1.155435 | | | (+) | | | |
| sequence AI448212) | | | | | | | |
| kinesin family member 21A | 0.854366 | | | (−) | (+) | RCC | DC |
| kise insert domain protein receptor | 0.839918 | | | (−) | (+) | RCC | DC |
| klotho | 0.469163 | 8E−04 | 0.5128 | (−) | (−) | RCC | C |
| Kruppel-like factor 1 (erythroid) | 0.688283 | | | (−) | | | |
| Kruppel-like factor 15 | 0.438157 | 8E−04 | 0.5538 | (−) | | | |
| Kruppel-like factor 5 | 1.315458 | | | (+) | (+) | RCC | C |
| Kruppel-like factor 9 | 0.582456 | 8E−04 | 0.5909 | (−) | | | |
| kynurenise (L-kynurenine hydrolase) | 0.745856 | | | (−) | | | |
| L-3-hydroxyacyl-Coenzyme A | 0.718971 | 0.004 | 0.6765 | (−) | (−) | RCC | C |
| dehydrogese, short chain | | | | | | | |
| lactate dehydrogese 1, A chain | 1.323347 | | | (+) | (+) | RCC | C |
| laminin B1 subunit 1 | 1.342184 | | | (+) | | | |
| laminin receptor 1 (67 kD, ribosomal | 1.663287 | 0.003 | 1.7401 | (+) | (+) | RCC | C |
| protein SA) | | | | | | | |
| laminin, alpha 2 | | 0.005 | 1.3048 | (+) | (+) | RCC | C |
| latexin | 1.246623 | | | (+) | (+) | RCC | C |
| lectin, galactose binding, soluble 3 | 3.883012 | 8E−04 | 2.5131 | (+) | (+) | RCC | C |
| lectin, galactose binding, soluble 4 | 0.732914 | | | (−) | | | |
| lectin, galactose binding, soluble 9 | 1.21399 | | | (+) | (+)/.(− | RCC | conflict |
| | | | | | ???) | | |
| leucine zipper-EF-hand containing | 0.740398 | 0.012 | 0.7633 | (−) | | | |
| transmembrane protein 1 | | | | | | | |
| leucocyte specific transcript 1 | | 0.012 | 1.3889 | (+) | (+) | RCC | C |
| leukemia-associated gene | 2.2171 | | | (+) | (+) | RCC | C |
| leukotriene C4 synthase | 1.287439 | | | (+) | | | |
| LIM and SH3 protein 1 | | 0.004 | 1.5453 | (+) | | | |
| lipoprotein lipase | 0.361706 | 0.001 | 0.5653 | (−) | (+) | RCC | DC |
| liver-specific bHLH-Zip transcription | | 0.004 | 1.3774 | (+) | | | |
| factor | | | | | | | |
| low density lipoprotein receptor- | 0.546832 | | | (−) | (−) | RCC | C |
| related protein 2 | | | | | | | |
| low density lipoprotein receptor- | 0.759073 | | | (−) | | | |
| related protein 6 | | | | | | | |
| LPS-induced TNF-alpha factor | 2.017366 | 8E−04 | 1.7774 | (+) | | | |
| lymphocyte antigen 6 complex, locus A | 1.627074 | | | (+) | | | |
| lymphocyte antigen 6 complex, locus E | 1.99767 | 8E−04 | 2.5458 | (+) | | | |
| lymphocyte specific 1 | 1.322083 | 0.003 | 2.0054 | (+) | (+) | RCC | C |
| lyric (D8Bwg1112e) D segment, Chr | | 0.048 | 1.2049 | (+) | | | |
| 8, Brigham & Women's Genetics | | | | | | | |
| 1112 expressed | | | | | | | |
| lysosomal-associated protein | | 0.025 | 1.2854 | (+) | | | |
| transmembrane 4A | | | | | | | |
| lysosomal-associated protein | | 8E−04 | 1.2595 | (+) | | | |
| transmembrane 4B | | | | | | | |
| lysosomal-associated protein | | 0.017 | 2.1031 | (+) | (+) | RCC | C |
| transmembrane 5 | | | | | | | |
| lysozyme | | 8E−04 | 5.7532 | (+) | (+) | RCC | C |
| lysyl oxidase-like | 1.390075 | | | (+) | | | |
| M. musculus mR for protein expressed | | 0.032 | 0.7977 | (−) | | | |
| at high levels in testis | | | | | | | |
| macrophage expressed gene 1 | 1.484724 | 8E−04 | 2.774 | (+) | | | |
| macrophage migration inhibitory | | 0.015 | 0.674 | (−) | | | |
| factor | | | | | | | |
| macrophage scavenger receptor 2 | | 8E−04 | 1.7086 | (+) | | | |
| MAD homolog 5 (Drosophila)/ | | 0.008 | 1.3266 | (+) | (+) | RCC | C |
| expressed sequence AI451355 | | | | | | | |
| mago-shi homolog, proliferation- | 1.277107 | | | (+) | (+) | RCC | C |
| associated (Drosophila) | | | | | | | |
| major vault protein | 1.428351 | | | (+) | | | |
| malate dehydrogese, soluble | 0.581342 | 8E−04 | 0.6478 | (−) | | | |
| malic enzyme, supertant | 0.683208 | 0.006 | 0.7935 | (−) | | | |
| malonyl-CoA decarboxylase | 0.635893 | 0.001 | 0.718 | (−) | | | |
| mammary tumor integration site 6 | 1.358134 | 0.009 | 1.3053 | (+) | (+) | RCC | C |
| mannose receptor, C type 1 | | 8E−04 | 1.738 | (+) | | | |
| mannose-6-pbosphate receptor, cation | | 0.025 | 1.3348 | (+) | | | |
| dependent | | | | | | | |
| MARCKS-like protein | | 8E−04 | 1.8277 | (+) | | | |
| matrix gamma-carboxyglutamate | 2.076147 | 8E−04 | 6.6453 | (+) | | | |
| (gla) protein | | | | | | | |
| matrix metalloproteise 14 | | 8E−04 | 2.0556 | (+) | (+) | RCC | C |
| (membrane-inserted) | | | | | | | |
| matrix metalloproteise 2 | | 0.002 | 1.5675 | (+) | (−) | RCC | DC |
| matrix metalloproteise 23 | | 0.019 | 1.2949 | (+) | | | |
| matrix metalloproteise 7 | | 0.014 | 1.921 | (+) | (+) | RCC | C |
| max binding protein | | 0.024 | 1.2911 | (+) | | | |
| melanoma antigen, family D, 2 | 1.25115 | 8E−04 | 1.3993 | (+) | | | |
| meprin 1 alpha | 0.603084 | 0.026 | 0.7488 | (−) | (+) | RCC | DC |
| metallothionein 1 | 1.799613 | 0.003 | 0.7041 | (+) | | | |
| metallothionein 2 | 2.336497 | | | (+) | (−) | RCC | DC |
| metastasis associated 1-like 1 | | 0.013 | 1.3714 | (+) | | | |
| methionine aminopeptidase 2 | 1.198553 | | | (+) | | | |
| methyl CpG binding protein 2 | | 0.011 | 0.8021 | (−) | | | |
| methylenetetrahydrofolate | 0.655893 | 0.004 | 0.6176 | (−) | (+) | RCC | DC |
| dehydrogese (DP+ dependent), | | | | | | | |
| methenyltetrahydrofolate | | | | | | | |
| cyclohydrolase, | | | | | | | |
| formyltetrahydrofolate synthase | | | | | | | |
| methylmalonyl-Coenzyme A mutase | 0.696844 | 0.042 | 0.7871 | (−) | | | |
| microfibrillar associated protein 5 | | 8E−04 | 1.4456 | (+) | | | |
| microtubule associated testis specific | 1.211841 | | | (+) | | | |
| serine/threonine protein kise | | | | | | | |
| microtubule-associated protein tau | 0.669051 | | | (−) | | | |
| microtubule-associated protein, | 1.295375 | | | (+) | | | |
| RP/EB family, member 1 | | | | | | | |
| mini chromosome maintence | 1.767788 | | | (+) | (+) | RCC | C |
| deficient (S. cerevisiae) | | | | | | | |
| mini chromosome maintence | 1.400229 | | | (+) | (+) | RCC | C |
| deficient 2 (S. cerevisiae) | | | | | | | |
| mini chromosome maintence | 1.61344 | | | (+) | (+) | RCC | C |
| deficient 4 homolog (S. cerevisiae) | | | | | | | |
| mini chromosome maintence | 1.676881 | | | (+) | (+) | RCC | C |
| deficient 7 (S. cerevisiae) | | | | | | | |
| mitochondrial ribosomal protein L39 | 0.61503 | | | (−) | | | |
| mitochondrial ribosomal protein L50; | 0.844369 | | | (−) | | | |
| (D4Wsu125e) D segment, Chr 4, | | | | | | | |
| Wayne State University 125, | | | | | | | |
| expressed | | | | | | | |
| Mitogen activated protein kinase 1; | 0.881133 | | | (−) | | | |
| RIKEN cD 9030612K14 gene | | | | | | | |
| mitogen activated protein kise 13 | 1.284772 | | | (+) | | | |
| mitogen activated protein kise kise | 1.44774 | | | (+) | | | |
| kise 1 | | | | | | | |
| mitogen-activated protein kise 7 | 1.154393 | | | (+) | | | |
| mitsugumin 29 | 0.746943 | | | (−) | | | |
| MORF-related gene X | 1.75411 | | | (+) | (+) | RCC | C |
| Muf1 protein (D630045E04Rik) Mus | | 0.029 | 1.3063 | (+) | | | |
| musculus, clone IMAGE: 3491421, | | | | | | | |
| mR, partial cds | | | | | | | |
| Mus musculus adult male kidney cD, | 0.83441 | | | (−) | | | |
| RIKEN full-length enriched library, | | | | | | | |
| clone:0610012C11:homogentisate 1, | | | | | | | |
| 2-dioxygese, full insert sequence | | | | | | | |
| Mus musculus adult male liver cD, | 0.497964 | | | (−) | | | |
| RIKEN full-length enriched library, | | | | | | | |
| clone:1300015E02:deoxyribonuclease | | | | | | | |
| II alpha, full insert sequence | | | | | | | |
| Mus musculus chemokine receptor | 0.684535 | 0.005 | 0.748 | (−) | | | |
| CCX CKR mR, complete cds, | | | | | | | |
| altertively spliced | | | | | | | |
| Mus musculus evectin-2 (Evt2) mR, | 0.708842 | | | (−) | | | |
| complete cds | | | | | | | |
| Mus musculus LDLR dan mR, | 0.768717 | | | (−) | | | |
| complete cds | | | | | | | |
| Mus musculus mR for 67 kDa | 1.237055 | | | (+) | | | |
| polymerase-associated factor PAF67 | | | | | | | |
| (paf67 gene) | | | | | | | |
| Mus musculus mR for alpha-albumin | 0.602557 | | | (−) | (−) | RCC | C |
| protein | | | | | | | |
| Mus musculus, basic transcription | 1.560713 | | | (+) | | | |
| factor 3, clone MGC: 6799 | | | | | | | |
| IMAGE: 2648048, mR, complete cds | | | | | | | |
| Mus musculus, clone | 0.81178 | | | (−) | | | |
| IMAGE: 3155544, mR, partial cds | | | | | | | |
| Mus musculus, clone | 1.496563 | 0.002 | 1.4937 | (+) | | | |
| IMAGE: 3494258, mR, partial cds | | | | | | | |
| Mus musculus, clone | 0.757009 | 0.043 | 0.7969 | (−) | | | |
| IMAGE: 3586777, mR, partial cds | | | | | | | |
| Mus musculus, clone | 0.627399 | | | (−) | | | |
| IMAGE: 3589087, mR, partial cds | | | | | | | |
| Mus musculus, clone | 0.81385 | | | (−) | | | |
| IMAGE: 3967158, mR, partial cds | | | | | | | |
| Mus musculus, clone | | 8E−04 | 1.6172 | (+) | | | |
| IMAGE: 3994696, mR, partial cds | | | | | | | |
| Mus musculus, clone | 1.225829 | | | (+) | | | |
| IMAGE: 4456744, mR, partial cds | | | | | | | |
| Mus musculus, clone | 1.530214 | | | (+) | | | |
| IMAGE: 4486265, mR, partial cds | | | | | | | |
| Mus musculus, clone | | 8E−04 | 2.1916 | (+) | | | |
| IMAGE: 4952483, mR, partial cds | | | | | | | |
| Mus musculus, clone | 0.695028 | | | (−) | (−) | RCC | C |
| IMAGE: 4974221, mR, partial cds | | | | | | | |
| Mus musculus, clone MGC: 12039 | 0.824624 | | | (−) | | | |
| IMAGE: 3603661, mR, complete cds | | | | | | | |
| Mus musculus, clone MGC: 12159 | | 0.014 | 1.3329 | (+) | | | |
| IMAGE: 3711169, mR, complete cds | | | | | | | |
| Mus musculus, clone MGC: 18871 | | 0.0103; | 0.6239812; | (−) | (−) | RCC | C |
| IMAGE: 4234793, mR, complete cds | | 0.0305 | 0.7169 | | | | |
| Mus musculus, clone MGC: 18985 | 1.364034 | | | (+) | (+) | RCC | C |
| IMAGE: 4011674, mR, complete cds | | | | | | | |
| Mus musculus, clone MGC: 19042 | 0.675484 | | | (−) | | | |
| IMAGE: 4188988, mR, complete cds | | | | | | | |
| Mus musculus, clone MGC: 19361 | 1.245176 | | | (+) | | | |
| IMAGE: 4242170, mR, complete cds | | | | | | | |
| Mus musculus, clone MGC: 29021 | 1.50073 | | | (+) | | | |
| IMAGE: 3495957, mR, complete cds | | | | | | | |
| Mus musculus, clone MGC: 36388 | 0.545973 | 0.006 | 0.6647 | (−) | | | |
| IMAGE: 5098924, mR, complete cds | | | | | | | |
| Mus musculus, clone MGC: 36554 | | 0.02 | 1.3223 | (+) | | | |
| IMAGE: 4954874, mR, complete cds | | | | | | | |
| Mus musculus, clone MGC: 36997 | 1.181755 | | | (+) | | | |
| IMAGE: 4948448, mR, complete cds | | | | | | | |
| Mus musculus, clone MGC: 37818 | 0.605546 | 0.022 | 0.6467 | (−) | | | |
| IMAGE: 5098655, mR, complete cds | | | | | | | |
| Mus musculus, clone MGC: 38363 | | 8E−04 | 1.5819 | (+) | (−) | RCC | DC |
| IMAGE: 5344986, mR, complete cds | | | | | | | |
| Mus musculus, clone MGC: 38798 | 0.804721 | | | (−) | | | |
| IMAGE: 5359803, mR, complete cds | | | | | | | |
| Mus musculus, clone MGC: 6377 | 1.153319 | | | (+) | | | |
| IMAGE: 3499365, mR, complete cds | | | | | | | |
| Mus musculus, clone MGC: 6545 | 0.719589 | | | (−) | (+) | RCC | DC |
| IMAGE: 2655444, mR, complete cds | | | | | | | |
| Mus musculus, clone MGC: 7898 | 0.640881 | 0.008 | 0.6501 | (−) | | | |
| IMAGE: 3582717, mR, complete cds | | | | | | | |
| Mus musculus, hypothetical protein | 0.834745 | | | (−) | | | |
| MGC11287 similar to ribosomal | | | | | | | |
| protein S6 kise,, clone MGC: 28043 | | | | | | | |
| IMAGE: 3672127, mR, complete cds | | | | | | | |
| Mus musculus, Similar to 60S | 0.854772 | | | (−) | | | |
| ribosomal protein L30 isolog, clone | | | | | | | |
| MGC: 6735 IMAGE: 3590401, mR, | | | | | | | |
| complete cds | | | | | | | |
| Mus musculus, Similar to | | 0.036 | 0.7253 | (−) | | | |
| angiopoietin-like factor, clone | | | | | | | |
| MGC: 32448 IMAGE: 5043159, mR, | | | | | | | |
| complete cds | | | | | | | |
| Mus musculus, Similar to CGI-147 | 1.221941 | 0.019 | 1.2422 | (+) | | | |
| protein, clone MGC: 25743 | | | | | | | |
| IMAGE: 3990061, mR, complete cds | | | | | | | |
| Mus musculus, Similar to | 0.783228 | 0.007 | 0.8377 | (−) | | | |
| chromosome 20 open reading frame | | | | | | | |
| 36, clone IMAGE: 5356821, mR, | | | | | | | |
| partial cds | | | | | | | |
| Mus musculus, Similar to cortactin | 1.340479 | | | (+) | | | |
| isoform B, clone MGC: 18474 | | | | | | | |
| IMAGE: 3981559, mR, complete cds | | | | | | | |
| Mus musculus, Similar to dendritic | 1.385299 | 0.046 | 1.3457 | (+) | | | |
| cell protein, clone MGC: 11741 | | | | | | | |
| IMAGE: 3969335, mR, complete cds | | | | | | | |
| Mus musculus, Similar to | | 8E−04 | 1.8677 | (+) | | | |
| DKFZP586B0621 protein, clone | | | | | | | |
| MGC: 38635 IMAGE: 5355789, mR, | | | | | | | |
| complete cds | | | | | | | |
| Mus musculus, similar to | 1.739406 | 0.01 | 1.3073 | (+) | | | |
| heterogeneous nuclear | | | | | | | |
| ribonucleoprotein A3 (H. sapiens), | | | | | | | |
| clone MGC: 37309 IMAGE: 4975085, | | | | | | | |
| mR, complete cds | | | | | | | |
| Mus musculus, Similar to | 1.338865 | | | (+) | | | |
| hypothetical protein | | | | | | | |
| DKFZp566A1524, clone MGC: 18989 | | | | | | | |
| IMAGE: 4012217, mR, complete cds | | | | | | | |
| Mus musculus, Similar to | 0.533357 | | | (−) | | | |
| hypothetical protein FLJ10520, clone | | | | | | | |
| MGC: 27888 IMAGE: 3497792, mR, | | | | | | | |
| complete cds | | | | | | | |
| Mus musculus, Similar to | 0.750638 | | | (−) | | | |
| hypothetical protein FLJ12618, clone | | | | | | | |
| MGC: 28775 IMAGE: 4487011, mR, | | | | | | | |
| complete cds | | | | | | | |
| Mus musculus, Similar to | 1.108571 | | | (+) | | | |
| hypothetical protein FLJ13213, clone | | | | | | | |
| MGC: 28555 IMAGE: 4206928, mR, | | | | | | | |
| complete cds | | | | | | | |
| Mus musculus, Similar to | | 8E−04 | 1.759 | (+) | | | |
| hypothetical protein FLJ20234, clone | | | | | | | |
| MGC: 37525 IMAGE: 4986113, mR, | | | | | | | |
| complete cds | | | | | | | |
| Mus musculus, Similar to | | 0.003 | 1.2319 | (+) | | | |
| hypothetical protein FLJ20245, clone | | | | | | | |
| MGC: 7940 IMAGE: 3584061, mR, | | | | | | | |
| complete cds | | | | | | | |
| Mus musculus, Similar to | 1.400228 | | | (+) | | | |
| hypothetical protein FLJ20335, clone | | | | | | | |
| MGC: 28912 IMAGE: 4922274, mR, | | | | | | | |
| complete cds | | | | | | | |
| Mus musculus, Similar to | 0.475177 | 0.036 | 0.6585 | (−) | | | |
| hypothetical protein FLJ21634, clone | | | | | | | |
| MGC: 19374 IMAGE: 2631696, mR, | | | | | | | |
| complete cds | | | | | | | |
| Mus musculus, Similar to | 1.337296 | | | (+) | | | |
| hypothetical protein MGC3133, clone | | | | | | | |
| MGC: 11596 IMAGE: 3965951, mR, | | | | | | | |
| complete cds | | | | | | | |
| Mus musculus, Similar to | | 0.004 | 0.7732 | (−) | | | |
| hypothetical protein MGC4368, clone | | | | | | | |
| MGC: 28978 IMAGE: 4503381, mR, | | | | | | | |
| complete cds | | | | | | | |
| Mus musculus, Similar to KIAA0763 | 0.804691 | | | (−) | | | |
| gene product, clone | | | | | | | |
| IMAGE: 4503056, mR, partial cds | | | | | | | |
| Mus musculus, Similar to KIAA1075 | 0.648409 | 8E−04 | 0.6346 | (−) | | | |
| protein, clone IMAGE: 5099327, mR, | | | | | | | |
| partial cds | | | | | | | |
| Mus musculus, Similar to MIPP65 | 0.720364 | | | (−) | | | |
| protein, clone MGC: 18783 | | | | | | | |
| IMAGE: 4188234, mR, complete cds | | | | | | | |
| Mus musculus, Similar to nucleolar | | 0.001 | 1.3895 | (+) | (+) | RCC | C |
| cysteine-rich protein, clone | | | | | | | |
| MGC: 6718 IMAGE: 3586161, mR, | | | | | | | |
| complete cds - pending | | | | | | | |
| Mus musculus, Similar to Protein P3, | | 0.003 | 1.2526 | (+) | | | |
| clone MGC: 38638 IMAGE: 5355849, | | | | | | | |
| mR, complete cds | | | | | | | |
| Mus musculus, similar to quinone | 0.5749 | | | (−) | | | |
| reductase-like protein, clone | | | | | | | |
| IMAGE: 4972406, mR, partial cds | | | | | | | |
| Mus musculus, similar to R29893_1, | 0.716169 | | | (−) | | | |
| clone MGC: 37808 IMAGE: 5098192, | | | | | | | |
| mR, complete cds | | | | | | | |
| Mus musculus, Similar to RAS p21 | 1.176812 | | | (+) | | | |
| protein activator, clone MGC: 7759 | | | | | | | |
| IMAGE: 3498774, mR, complete cds | | | | | | | |
| Mus musculus, Similar to retinol | 0.48924 | | | (−) | | | |
| dehydrogese type 6, clone | | | | | | | |
| MGC: 25965 IMAGE: 4239862, mR, | | | | | | | |
| complete cds | | | | | | | |
| Mus musculus, Similar to ribosomal | | 8E−04 | 1.6264 | (+) | | | |
| protein S20, clone MGC: 6876 | | | | | | | |
| IMAGE: 2651405, mR, complete cds | | | | | | | |
| Mus musculus, Similar to sirtuin | 0.828673 | | | (−) | | | |
| silent mating type information | | | | | | | |
| regulation 2 homolog 7 (S. cerevisiae), | | | | | | | |
| clone MGC: 37560 | | | | | | | |
| IMAGE: 4987746, mR, complete cds | | | | | | | |
| Mus musculus, Similar to transgelin | 2.078132 | 8E−04 | 1.8563 | (+) | (+) | RCC | C |
| 2, clone MGC: 6300 | | | | | | | |
| IMAGE: 2654381, mR, complete cds | | | | | | | |
| Mus musculus, Similar to ubiquitin- | 0.669748 | 8E−04 | 0.6707 | (−) | (+) | RCC | DC |
| conjugating enzyme E2 variant 1, | | | | | | | |
| clone MGC: 7660 IMAGE: 3496088, | | | | | | | |
| mR, complete cds | | | | | | | |
| Mus musculus, Similar to unc93 | | 8E−04 | 2.1075 | (+) | | | |
| (C. elegans) homolog B, clone | | | | | | | |
| MGC: 25627 IMAGE: 4209296, mR, | | | | | | | |
| complete cds | | | | | | | |
| Mus musculus, Similar to xylulokise | 0.63543 | 0.023 | 0.6757 | (−) | | | |
| homolog (H. influenzae), clone | | | | | | | |
| IMAGE: 5043428, mR, partial cds | | | | | | | |
| mutS homolog 2 (E. coli) | 1.173315 | | | (+) | (+) | RCC | C |
| mutS homolog 6 (E. coli) | 1.287113 | | | (+) | | | |
| MYB binding protein (P160) 1a | 1.37183 | | | (+) | | | |
| MYC-associated zinc finger protein | 1.330611 | | | (+) | (+) | RCC | C |
| (purine-binding transcription factor) | | | | | | | |
| myelocytomatosis oncogene | 1.459356 | 0.014 | 1.4883 | (+) | (+) | RCC | C |
| myeloid differentiation primary | | 0.004 | 1.441 | (+) | | | |
| response gene 88 | | | | | | | |
| myeloid-associated differentiation | 1.390891 | | | (+) | | | |
| marker | | | | | | | |
| myocyte enhancer factor 2A | | 0.009 | 1.2539 | (+) | (+)/(−) | RCC | conflict |
| myosin Ic | 1.288644 | | | (+) | | | |
| myosin light chain, alkali, cardiac | 1.622514 | | | (+) | | | |
| atria | | | | | | | |
| myosin light chain, alkali, nonmuscle | | 0.028 | 1.4658 | (+) | (−) | RCC | DC |
| myristoylated alanine rich protein | | 8E−04 | 1.8458 | (+) | | | |
| kise C substrate | | | | | | | |
| N-acetylglucosamine kise | 1.23848 | | | (+) | (+) | RCC | C |
| N-acetylneuramite pyruvate lyase | 1.325459 | | | (+) | | | |
| NCK-associated protein 1 | | 0.004 | 1.4471 | (+) | | | |
| nestin - pendin | 1.226027 | | | (+) | | | |
| neural precursor cell expressed, | | 0.004 | 0.7168 | (−) | | | |
| developmentally down-regulated gene | | | | | | | |
| 4a | | | | | | | |
| neural proliferation, differentiation | 1.34827 | 0.037 | 1.263 | (+) | (+) | RCC | C |
| and control gene 1 | | | | | | | |
| neurol guanine nucleotide exchange | 0.773454 | | | (−) | | | |
| factor | | | | | | | |
| neuropilin | | 0.031 | 1.3972 | (+) | (+) | RCC | C |
| neutrophil cytosolic factor 2 | 1.233541 | | | (+) | | | |
| Ngfi-A binding protein 2 | | 0.049 | 1.2723 | (+) | | | |
| nicotimide nucleotide transhydrogese | 0.542394 | 8E−04 | 0.5672 | (−) | (−) | RCC | C |
| nidogen 1 | | 0.003 | 1.5346 | (+) | (+) | RCC | C |
| NIMA (never in mitosis gene a)- | 1.464337 | | | (+) | | | |
| related expressed kise 6 | | | | | | | |
| N-myc downstream regulated 2 | 0.598324 | 0.003 | 0.7062 | (−) | | | |
| non-catalytic region of tyrosine kise | | 0.005 | 1.3379 | (+) | (+) | RCC | C |
| adaptor protein 1 | | | | | | | |
| nuclear factor of kappa light chain | | 0.009 | 1.4106 | (+) | | | |
| gene enhancer in B-cells 1, p105 | | | | | | | |
| nuclear protein 15.6 | 0.771762 | | | (−) | | | |
| nuclear receptor coactivator 4 | | 0.034 | 0.6812 | (−) | (+) | RCC | DC |
| nuclear receptor subfamily 2, group | | 0.011 | 1.3455 | (+) | (+) | RCC | C |
| F, member 2 | | | | | | | |
| nuclear receptor subfamily 2, group | | 0.036 | 1.2859 | (+) | (−) | RCC | DC |
| F, member 6 | | | | | | | |
| nuclease sensitive element binding | 1.47757 | | | (+) | (+) | RCC | C |
| protein 1 | | | | | | | |
| nucleophosmin 1 | 1.441561 | 8E−04 | 1.6685 | (+) | (+) | RCC | C |
| numb gene homolog (Drosophila) | 1.591483 | | | (+) | | | |
| oncostatin receptor | 1.348268 | 8E−04 | 2.0715 | (+) | | | |
| opioid growth factor receptor | 1.198578 | | | (+) | | | |
| ornithine aminotransferase | | 0.022 | 0.7587 | (−) | | | |
| ornithine decarboxylase, structural | 1.312592 | | | (+) | | | |
| osteomodulin | 0.828403 | | | (−) | | | |
| oxysterol binding protein-like 1A | 0.670761 | 0.01 | 0.6983 | (−) | | | |
| pantophysin | 0.644709 | 9E−04 | 0.6323 | (−) | | | |
| papillary rel cell carcinoma | | 0.002 | 1.4613 | (+) | (?) | RCC | conflict |
| (translocation-associated) | | | | | | | |
| parvalbumin | 0.507541 | | | (−) | (+)/(−) | RCC | conflict |
| PC4 and SFRS1 interacting protein 2 | 1.201167 | | | (+) | | | |
| (expressed sequence AU015605) | | | | | | | |
| PCTAIRE-motif protein kise 3 | 0.808356 | | | (−) | (+) | RCC | DC |
| peptidylprolyl isomerase | 1.194882 | | | (+) | (+) | RCC | C |
| (cyclophilin)-like 1 | | | | | | | |
| peptidylprolyl isomerase C | 0.855714 | | | (−) | | | |
| peptidylprolyl isomerase C-associated | | 0.004 | 1.6664 | (+) | (+) | RCC | C |
| protein | | | | | | | |
| period homolog 1 (Drosophila) | | 0.0008; | 0.5522979; | (−) | | | |
| | | 0.0305 | 0.7390266 | | | | |
| period homolog 2 (Drosophila) | | 0.005 | 0.6496 | (−) | | | |
| peroxiredoxin 5 | 1.36499 | | | (+) | (?) | RCC | conflict |
| peroxisomal biogenesis factor 13 | 0.827587 | | | (−) | | | |
| peroxisomal delta3, delta2-enoyl- | 0.732094 | | | (−) | (−) | RCC | C |
| Coenzyme A isomerase | | | | | | | |
| peroxisomal membrane protein 2, 22 kDa | 0.671027 | | | (−) | (+)/(−) | RCC | conflict |
| peroxisomal sarcosine oxidase | 0.675459 | | | (−) | (−) | RCC | C |
| peroxisome proliferator activated | 0.605623 | | | (−) | | | |
| receptor alpha | | | | | | | |
| PH domain containing protein in reti 1 | 0.770569 | | | (−) | | | |
| phenylalanine hydroxylase | 0.483001 | 8E−04 | 0.4244 | (−) | (−) | RCC | C |
| phenylalkylamine Ca2+ antagonist | 0.701194 | | | (−) | | | |
| (emopamil) binding protein | | | | | | | |
| phorbol-12-myristate-13-acetate- | 1.320285 | 0.047 | 1.3734 | (+) | | | |
| induced protein 1 | | | | | | | |
| phosphatidylinositol 3-kise, | 1.234427 | | | (+) | | | |
| regulatory subunit, polypeptide 1 | | | | | | | |
| (p85 alpha) | | | | | | | |
| phosphatidylinositol transfer protein | 1.356671 | | | (+) | | | |
| phosphodiesterase 1A, calmodulin- | 0.832816 | | | (−) | (−) | RCC | C |
| dependent | | | | | | | |
| phosphofructokise, liver, B-type | 0.836516 | | | (−) | | | |
| phosphoglycerate kise 1 | 0.83983 | | | (−) | (+) | RCC | DC |
| phosphoglycerate mutase 2 | 0.435688 | 0.044 | 0.6904 | (−) | | | |
| phospholipase A2, activating protein | 1.249295 | | | (+) | | | |
| phospholipase A2, group IB, pancreas | 1.706747 | | | (+) | | | |
| phospholipase A2, group IIA | 0.841435 | | | (−) | | | |
| (platelets, synovial fluid) | | | | | | | |
| phospholipid scramblase 1 | 1.634313 | | | (+) | (+) | RCC | C |
| phosphoprotein enriched in astrocytes | 2.04807 | | | (+) | (+) | RCC | C |
| 15 | | | | | | | |
| phytanoyl-CoA hydroxylase | 0.706937 | | | (−) | (−) | RCC | C |
| plasminogen activator, tissue | | 0.02 | 1.423 | (+) | (−) | RCC | DC |
| platelet derived growth factor | 1.386991 | | | (+) | | | |
| receptor, beta polypeptide | | | | | | | |
| platelet derived growth factor, alpha | | 0.014 | 1.327 | (+) | | | |
| platelet derived growth factor, B | | 8E−04 | 1.6569 | (+) | (+) | RCC | C |
| polypeptide | | | | | | | |
| platelet factor 4 | 1.959063 | 0.036 | 1.5766 | (+) | | | |
| platelet-activating factor | | 8E−04 | 1.462 | (+) | | | |
| acetylhydrolase, isoform 1b, alpha1 | | | | | | | |
| subunit | | | | | | | |
| poliovirus receptor-related 3 | 1.277304; | | | (+) | (+) | RCC | C |
| | 1.163199 | | | | | | |
| poly (A) polymerase alpha | 0.455758 | 0.009 | 0.6839 | (−) | (+) | RCC | DC |
| poly(rC) binding protein 1 | 1.229561 | | | (+) | (+) | RCC | C |
| polycystic kidney disease 1 homolog | 0.861306 | | | (−) | (+) | RCC | DC |
| polymerase, gamma | | 0.041 | 0.758 | (−) | | | |
| polypyrimidine tract binding protein 1 | 1.187485 | | | (+) | (+) | RCC | C |
| potassium channel, subfamily K, | 0.816677 | | | (−) | | | |
| member 2 | | | | | | | |
| PPAR gamma coactivator-1beta | 0.752031 | | | (−) | | | |
| protein | | | | | | | |
| prion protein | | 0.015 | 0.6883 | (−) | | | |
| procollagen lysine, 2-oxoglutarate 5- | 1.236481 | | | (+) | (+) | RCC | C |
| dioxygese 2 | | | | | | | |
| procollagen, type I, alpha 1 | | 8E−04 | 4.1081 | (+) | (+)/(−?) | RCC | conflict |
| procollagen, type I, alpha 2 | | 8E−04 | 2.8442 | (+) | (+) | RCC | C |
| procollagen, type IV, alpha 1 | 1.962618 | 0.003 | 2.2032 | (+) | (+) | RCC | C |
| procollagen, type IV, alpha 2 | | 0.032 | 1.8088 | (+) | (+) | RCC | C |
| procollagen, type V, alpha 1 | 1.363199 | | | (+) | (+) | RCC | C |
| procollagen, type V, alpha 2 | 1.555847 | 8E−04 | 1.4432 | (+) | (+) | RCC | C |
| prohibitin | 0.875224 | | | (−) | | | |
| proline dehydrogese | 0.555697 | 8E−04 | 0.5546 | (−) | | | |
| protease (prosome, macropain) 26S | 1.274107 | | | (+) | | | |
| subunit, ATPase 1 | | | | | | | |
| proteaseome (prosome, macropain) | 0.545487 | | | (−) | | | |
| 28 subunit, 3 | | | | | | | |
| proteasome (prosome, macropain) | 1.249655 | | | (+) | | | |
| 26S subunit, non-ATPase, 10 | | | | | | | |
| proteasome (prosome, macropain) | 1.274187 | | | (+) | (+) | RCC | C |
| 26S subunit, non-ATPase, 13 | | | | | | | |
| proteasome (prosome, macropain) 28 | 1.412928 | 9E−04 | 1.7167 | (+) | | | |
| subunit, alpha | | | | | | | |
| proteasome (prosome, macropain) | 1.318854 | | | (+) | | | |
| subunit, alpha type 2 | | | | | | | |
| proteasome (prosome, macropain) | 1.252206 | | | (+) | (+) | RCC | C |
| subunit, alpha type 6 | | | | | | | |
| proteasome (prosome, macropain) | | 0.013 | 1.3768 | (+) | (+) | RCC | C |
| subunit, alpha type 7 | | | | | | | |
| proteasome (prosome, macropain) | | 0.015 | 1.3622 | (+) | | | |
| subunit, beta type 1 | | | | | | | |
| proteasome (prosome, macropain) | | 0.003 | 1.5053 | (+) | (+) | RCC | C |
| subunit, beta type 10 | | | | | | | |
| protein C | 0.716043 | | | (−) | (−) | RCC | C |
| protein kise C, delta | | 0.009 | 1.3244 | (+) | (+) | RCC | C |
| protein phosphatase 1, catalytic | 1.477029 | | | (+) | | | |
| subunit, alpha isoform | | | | | | | |
| protein phosphatase 1, regulatory | 0.393414 | | | (−) | | | |
| (inhibitor) subunit 1A | | | | | | | |
| protein phosphatase 2a, catalytic | 1.289147 | | | (+) | (−) | RCC | DC |
| subunit, beta isoform | | | | | | | |
| protein phosphatase 3, catalytic | 0.858408 | | | (−) | | | |
| subunit, gamma isoform | | | | | | | |
| protein S (alpha) | | 8E−04 | 1.7106 | (+) | | | |
| protein tyrosine phosphatase 4a1 | 1.499428 | | | (+) | | | |
| protein tyrosine phosphatase, non- | 1.212579 | 0.038 | 1.2656 | (+) | | | |
| receptor type 9 | | | | | | | |
| protein tyrosine phosphatase, receptor | 0.830019 | | | (−) | (+) | RCC | DC |
| type, B | | | | | | | |
| protein tyrosine phosphatase, receptor | 1.214849 | 0.002 | 1.5928 | (+) | | | |
| type, C | | | | | | | |
| protein tyrosine phosphatase, receptor | | 0.001 | 1.6535 | (+) | | | |
| type, C polypeptide-associated | | | | | | | |
| protein | | | | | | | |
| protein tyrosine phosphatase, receptor | | 0.007 | 1.2743 | (+) | (−) | RCC | DC |
| type, O | | | | | | | |
| proteoglycan, secretory granule | 1.368298 | | | (+) | (+) | RCC | C |
| proteosome (prosome, macropain) | | 0.005 | 1.8412 | (+) | (+) | RCC | C |
| subunit, beta type 8 (large | | | | | | | |
| multifunctiol protease 7) | | | | | | | |
| prothymosin alpha | 1.383187 | 8E−04 | 1.5311 | (+) | (+) | RCC | C |
| purinergic receptor (family A group | | 0.029 | 1.2282 | (+) | | | |
| 5); RIKEN cD 2610302I02 gene | | | | | | | |
| pyridoxal (pyridoxine, vitamin B6) | 1.569586 | | | (+) | | | |
| kise | | | | | | | |
| PYRIN-containing APAF1-like | | 0.005 | 0.6865 | (−) | | | |
| protein 5/expressed sequence | | | | | | | |
| AI504961 | | | | | | | |
| pyruvate decarboxylase | | 0.026 | 0.6537 | (−) | | | |
| pyruvate dehydrogese 2 | 0.566341 | | | (−) | | | |
| pyruvate kise 3 | 1.368806 | | | (+) | | | |
| pyruvate kise liver and red blood cell | 0.83514 | 0.004 | 0.7669 | (−) | (−) | RCC | C |
| R binding motif protein 3 | 2.299533 | 8E−04 | 1.6893 | (+) | | | |
| R polymerase I associated factor, 53 kD | 1.348222 | | | (+) | | | |
| R polymerase II 1 | 0.808996 | | | (−) | | | |
| RAB11a, member RAS oncogene | 1.160313 | | | (+) | (+) | RCC | C |
| family | | | | | | | |
| RAB3D, member RAS oncogene | | 0.013 | 1.212 | (+) | | | |
| family | | | | | | | |
| Ral-interacting protein 1 | 1.278257 | | | (+) | (−) | RCC | DC |
| RAN, member RAS oncogene family | 2.1891 | | | (+) | (+) | RCC | C |
| Rap1, GTPase-activating protein 1 | 0.584864 | | | (−) | (−) | RCC | C |
| RAR-related orphan receptor alpha | | 0.046 | 0.7432 | (−) | | | |
| ras homolog 9 (RhoC) | 1.757009 | 0.004 | 1.9305 | (+) | | | |
| ras homolog B (RhoB) | 1.550957 | 0.029 | 1.4336 | (+) | (+) | RCC | C |
| ras homolog D (RhoD) | | 0.004 | 1.3517 | (+) | | | |
| ras homolog gene family, member E | 0.785447 | | | (−) | (+) | RCC | DC |
| Ras-GTPase-activating protein | 1.196988 | | | (+) | | | |
| (GAP<120>) SH3-domain binding | | | | | | | |
| protein 2 | | | | | | | |
| RAS-related C3 botulinum substrate 2 | | 0.049 | 1.5523 | (+) | | | |
| reduced expression 3 | | 0.003 | 0.6367 | (−) | | | |
| regulator for ribosome resistance | 1.295449 | | | (+) | | | |
| homolog (S. cerevisiae) | | | | | | | |
| regulator of G-protein sigling 14 | 1.320308 | 0.034 | 1.2757 | (+) | | | |
| regulator of G-protein sigling 19 | 1.236906 | | | (+) | | | |
| interacting protein 1 | | | | | | | |
| renin 2 tandem duplication of Ren1 | | 0.008 | 0.6953 | (−) | | | |
| reticulocalbin | 1.439527 | | | (+) | (+) | RCC | C |
| reticulon 3 | 0.790275 | | | (−) | (+) | RCC | DC |
| retinoblastoma binding protein 4 | | 0.049 | 1.2221 | (+) | | | |
| retinoblastoma binding protein 7 | 1.357157 | | | (+) | (+) | RCC | C |
| retinoblastoma-like 1 (p107) | 1.374764 | | | (+) | | | |
| retinoic acid early transcript gamma | | 0.004 | 1.6762 | (+) | | | |
| retinoic acid induced 1 | 1.181703 | | | (+) | | | |
| retinol binding protein 1, cellular | | 8E−04 | 1.8488 | (+) | | | |
| Rhesus blood group-associated C | 0.656037 | | | (−) | | | |
| glycoprotein | | | | | | | |
| Rho guanine nucleotide exchange | 0.849341 | | | (−) | | | |
| factor (GEF) 3 | | | | | | | |
| ribonucleotide reductase M1 | 0.733893 | | | (−) | (+) | RCC | DC |
| ribosomal protein L10A | 1.983487 | 0.014 | 1.7402 | (+) | (+) | RCC | C |
| ribosomal protein L12 | | 8E−04 | 2.0943 | (+) | (+) | RCC | C |
| ribosomal protein L13a | 1.991657 | | | (+) | (+) | RCC | C |
| ribosomal protein L18 | | 0.003 | 1.6779 | (+) | (+) | RCC | C |
| ribosomal protein L19 | 1.808252 | 0.049 | 1.543 | (+) | (+) | RCC | C |
| ribosomal protein L21 | 1.514015 | | | (+) | (+) | RCC | C |
| ribosomal protein L27a | 1.615386 | 0.004 | 1.5963 | (+) | (+) | RCC | C |
| ribosomal protein L28 | 1.580825 | | | (+) | (+) | RCC | C |
| ribosomal protein L29 | 1.556484 | 0.008 | 1.6119 | (+) | (+) | RCC | C |
| ribosomal protein L3 | 1.589752 | 0.001 | 1.5617 | (+) | | | |
| ribosomal protein L35 | 1.949571 | 0.003 | 1.7314 | (+) | | | |
| ribosomal protein L36 | 1.542536 | | | (+) | (+) | RCC | C |
| ribosomal protein L41 | 1.766693 | | | (+) | (+) | RCC | C |
| ribosomal protein L44 | 1.990451 | 9E−04 | 1.5496 | (+) | | | |
| ribosomal protein L5 | 1.811149 | 8E−04 | 1.4804 | (+) | | | |
| ribosomal protein L6 | 1.885371 | 0.009 | 1.3565 | (+) | (+) | RCC | C |
| ribosomal protein L7 | | 0.012 | 1.807 | (+) | (+) | RCC | C |
| ribosomal protein L8 | 1.476231 | | | (+) | (+) | RCC | C |
| ribosomal protein S14 | | 0.004 | 1.7229 | (+) | (+) | RCC | C |
| ribosomal protein S15 | 1.867474 | 8E−04 | 1.6115 | (+) | | | |
| ribosomal protein S15 | 1.566886 | | | (+) | | | |
| ribosomal protein S16 | 1.95787 | 0.001 | 1.572 | (+) | (+) | RCC | C |
| ribosomal protein S19 | 1.616338 | | | (+) | (+) | RCC | C |
| ribosomal protein S2 | 1.8787 | | | (+) | (+) | RCC | C |
| ribosomal protein S23 | 1.379952 | 8E−04 | 1.4732 | (+) | (+) | RCC | C |
| ribosomal protein S26 | 1.468534 | | | (+) | | | |
| ribosomal protein S29 | | 0.027 | 1.4417 | (+) | | | |
| ribosomal protein S3 | 1.528904 | | | (+) | (+) | RCC | C |
| ribosomal protein S3a | 1.878501 | 8E−04 | 1.4223 | (+) | (+) | RCC | C |
| ribosomal protein S4, X-linked | 1.873272 | 8E−04 | 1.607 | (+) | | | |
| ribosomal protein S5 | | 8E−04 | 1.9502 | (+) | | | |
| ribosomal protein S6 | 1.637744; | 0.0008; | 1.416617; | (+) | | | |
| | 1.663683 | 0.0251 | 1.63716 | | | | |
| ribosomal protein S6 kise, 90 kD, | 1.345873 | | | (+) | | | |
| polypeptide 4 | | | | | | | |
| ribosomal protein S7 | 1.886875 | 0.002 | 1.6322 | (+) | | | |
| ribosomal protein, large P2 | | 0.004 | 1.4626 | (+) | (+) | RCC | C |
| ribosomal protein, large, P1 | 2.003644 | 0.029 | 1.7745 | (+) | (+) | RCC | C |
| RIKEN cD 0610006F02 gene | | 0.0008; | 0.6493102; | (−) | | | |
| | | 0.0489 | 0.7666818 | | | | |
| RIKEN cD 0610006N12 gene | 0.783579 | | | (−) | | | |
| RIKEN cD 0610007L01 gene | 1.194059 | | | (+) | | | |
| RIKEN cD 0610011C19 gene | 0.753575 | | | (−) | | | |
| RIKEN cD 0610016J10 gene | 1.384281 | | | (+) | | | |
| RIKEN cD 0610025G13 gene | 1.618142 | 0.004 | 1.4677 | (+) | (−)/(+) | RCC | conflict |
| RIKEN cD 0610025I19 gene | 0.573976 | 0.044 | 0.7207 | (−) | | | |
| RIKEN cD 0610041E09 gene | 1.318886 | | | (+) | | | |
| RIKEN cD 1010001M04 gene | 0.701714 | | | (−) | | | |
| RIKEN cD 1100001F19 gene | 1.367751 | | | (+) | | | |
| RIKEN cD 1100001J13 gene - | 0.821539 | | | (−) | (+) | RCC | DC |
| pending | | | | | | | |
| RIKEN cD 1110001I24 gene | 1.385664 | 0.029 | 1.2197 | (+) | | | |
| RIKEN cD 1110002C08 gene | 0.801259 | | | (−) | | | |
| RIKEN cD 1110005N04 gene | | 0.012 | 1.2392 | (+) | | | |
| RIKEN cD 1110007F23 gene | | 0.007 | 1.2275 | (+) | | | |
| RIKEN cD 1110008B24 gene | | 0.002 | 1.3502 | (+) | | | |
| RIKEN cD 1110014C03 gene | 1.449833 | | | (+) | | | |
| RIKEN cD 1110020L19 gene | 1.199686 | | | (+) | | | |
| RIKEN cD 1110032A13 gene | | 8E−04 | 1.9945 | (+) | | | |
| RIKEN cD 1110038J12 gene | 0.786088 | 0.01 | 0.7623 | (−) | | | |
| RIKEN cD 1110038L14 gene | 1.460735 | | | (+) | (+) | RCC | C |
| RIKEN cD 1110054A24 gene | 1.386487 | | | (+) | | | |
| RIKEN cD 1190006C12 gene | | 0.002 | 1.5092 | (+) | | | |
| RIKEN cD 1200003E16 gene | 0.827166 | | | (−) | | | |
| RIKEN cD 1200009B18 gene | | 0.013 | 1.3411 | (+) | | | |
| RIKEN cD 1200011D11 gene | 0.569291 | | | (−) | | | |
| RIKEN cD 1200013A08 gene | | 8E−04 | 1.549 | (+) | | | |
| RIKEN cD 1200014D15 gene | 0.489823 | 0.031 | 0.6793 | (−) | | | |
| RIKEN cD 1200014I03 gene | 1.383879 | | | (+) | | | |
| RIKEN cD 1200015A22 gene | 1.226764 | | | (+) | | | |
| RIKEN cD 1200016G03 gene | 0.828808 | | | (−) | | | |
| RIKEN cD 1300002P22 gene | 0.510225 | | | (−) | | | |
| RIKEN cD 1300004O04 gene | 0.761224 | 0.005 | 0.7406 | (−) | | | |
| RIKEN cD 1300013F15 gene | | 0.021 | 0.684 | (−) | | | |
| RIKEN cD 1300013G12 gene | 1.228874 | | | (+) | | | |
| RIKEN cD 1300017C12 gene | 0.785174 | | | (−) | (−) | RCC | C |
| RIKEN cD 1300018I05 gene | 1.252751 | | | (+) | | | |
| RIKEN cD 1300019I21 gene | 1.245337 | | | (+) | | | |
| RIKEN cD 1500010B24 gene | | 0.002; | 1.398499; | (+) | (+) | RCC | C |
| | | 0.002 | 1.411263 | | | | |
| RIKEN cD 1500026A19 gene | 1.180374 | | | (+) | | | |
| RIKEN cD 1500041J02 gene | 0.781326 | 0.04 | 0.7179 | (−) | | | |
| RIKEN cD 1700008H23 gene | | 0.029 | 0.8204 | (−) | | | |
| RIKEN cD 1700012B18 gene | 0.660943 | | | (−) | | | |
| RIKEN cD 1700015P13 gene | | 0.04 | 0.7114 | (−) | | | |
| RIKEN cD 1700016A15 gene | | 0.026 | 1.2838 | (+) | | | |
| RIKEN cD 1700028A24 gene | 0.705073 | | | (−) | | | |
| RIKEN cD 1700037H04 gene | 1.138844 | | | (+) | | | |
| RIKEN cD 1810009M01 gene | 2.104826 | | | (+) | | | |
| RIKEN cD 1810013B01 gene | 0.61166 | | | (−) | | | |
| RIKEN cD 1810023B24 gene | 1.264664 | | | (+) | | | |
| RIKEN cD 1810027P18 gene | 0.601175 | | | (−) | (−) | RCC | C |
| RIKEN cD 1810036E22 gene | 0.70486 | | | (−) | | | |
| RIKEN cD 1810038D15 gene | 1.282694 | | | (+) | | | |
| RIKEN cD 1810043O07 gene | | 0.004 | 1.2972 | (+) | | | |
| RIKEN cD 1810054O13 gene | 0.67673 | | | (−) | | | |
| RIKEN cD 1810058K22 gene | 1.378858 | | | (+) | | | |
| RIKEN cD 2010012D11 gene | 0.716885 | 0.003 | 0.6902 | (−) | | | |
| RIKEN cD 2010315L10 gene | 1.204993 | | | (+) | | | |
| RIKEN cD 2310001A20 gene | 0.726674 | | | (−) | | | |
| RIKEN cD 2310004I03 gene | 0.812809 | | | (−) | | | |
| RIKEN cD 2310004L02 gene | 0.767893 | 0.009 | 0.7563 | (−) | | | |
| RIKEN cD 2310009E04 gene | 0.619409 | 0.03 | 0.7724 | (−) | | | |
| RIKEN cD 2310010G13 gene | 0.90919 | | | (−) | | | |
| RIKEN cD 2310022K15 gene | | 0.042 | 1.2791 | (+) | | | |
| RIKEN cD 2310032J20 gene | 0.456694 | | | (−) | | | |
| RIKEN cD 2310046G15 gene | | 0.013 | 1.3684 | (+) | (+) | RCC | C |
| RIKEN cD 2310051E17 gene | 0.616314 | | | (−) | | | |
| RIKEN cD 2310067B10 gene | 0.805886 | | | (−) | | | |
| RIKEN cD 2310075M15 gene | 1.253001 | 0.0290 | 1.3141 | (+) | | | |
| RIKEN cD 2310079C17 gene | 1.178546 | | | (+) | | | |
| RIKEN cD 2410002J21 gene | 1.358002 | | | (+) | | | |
| RIKEN cD 2410021P16 gene | 0.679461 | | | (−) | | | |
| RIKEN cD 2410026K10 gene | | 8E−04 | 1.9506 | (+) | | | |
| RIKEN cD 2410029D23 gene | 0.774382 | | | (−) | | | |
| RIKEN cD 2410129E14 gene | | 8E−04 | 2.0517 | (+) | | | |
| RIKEN cD 2410174K12 gene | | 0.036 | 1.3316 | (+) | | | |
| RIKEN cD 2510015F01 gene | 1.566621 | | | (+) | | | |
| RIKEN cD 2600001N01 gene | 1.259811 | | | (+) | | | |
| RIKEN cD 2600015J22 gene | | 0.004 | 1.6201 | (+) | | | |
| RIKEN cD 2600017H24 gene | 1.480539 | | | (+) | | | |
| RIKEN cD 2610007A16 gene | 0.706068 | | | (−) | | | |
| RIKEN cD 2610029K21 gene | 1.159174 | | | (+) | | | |
| RIKEN cD 2610039E05 gene | 0.776991 | | | (−) | | | |
| RIKEN cD 2610200M23 gene | | 0.003 | 1.4284 | (+) | (+) | RCC | C |
| RIKEN cD 2610206D03 gene | 1.27124 | | | (+) | | | |
| RIKEN cD 2610301D06 gene | 1.849151 | | | (+) | | | |
| RIKEN cD 2610305D13 gene | 2.013008 | | | (+) | | | |
| RIKEN cD 2610306D21 gene | | 0.038 | 1.3795 | (+) | | | |
| RIKEN cD 2610511O17 gene | 1.177157 | | | (+) | | | |
| RIKEN cD 2610524G07 gene | 0.702826 | | | (−) | | | |
| RIKEN cD 2610524G09 gene | 1.175638 | | | (+) | | | |
| RIKEN cD 2700027J02 gene | 1.235225 | | | (+) | | | |
| RIKEN cD 2700038K18 gene | | 0.003 | 1.5276 | (+) | | | |
| RIKEN cD 2700038M07 gene - | | 8E−04 | 1.9098 | (+) | (−) | RCC | DC |
| pending | | | | | | | |
| RIKEN cD 2700055K07 gene | | 0.029 | 1.3762 | (+) | | | |
| RIKEN cD 2700099C19 gene | 1.141995 | | | (+) | | | |
| RIKEN cD 2810004N23 gene | 1.296022 | | | (+) | | | |
| RIKEN cD 2810047L02 gene | 1.371268 | | | (+) | | | |
| RIKEN cD 2810409H07 gene | 1.352519 | | | (+) | | | |
| RIKEN cD 2810411G23 gene | 1.327569 | | | (+) | (+) | RCC | C |
| RIKEN cD 2810418N01 gene | | 0.004 | 1.4296 | (+) | | | |
| RIKEN cD 2810430J06 gene | | 0.038 | 1.3085 | (+) | | | |
| RIKEN cD 2810468K17 gene | | 0.022 | 1.185 | (+) | | | |
| RIKEN cD 2810473M14 gene | 0.624595 | | | (−) | | | |
| RIKEN cD 2900074L19 gene | | 0.049 | 0.706 | (−) | | | |
| RIKEN cD 3010001A07 gene | 0.829789 | | | (−) | | | |
| RIKEN cD 3010027G13 gene | 0.765137 | | | (−) | | | |
| RIKEN cD 3021401A05 gene | 1.605988 | 8E−04 | 3.0674 | (+) | | | |
| RIKEN cD 3110001N18 gene | | 9E−04 | 1.3959 | (+) | (+) | RCC | C |
| RIKEN cD 3230402E02 gene | 1.291597 | | | (+) | (+) | RCC | C |
| RIKEN cD 3321401G04 gene | | 0.029 | 1.3004 | (+) | | | |
| RIKEN cD 4430402G14 gene | 1.473069 | 8E−04 | 1.4996 | (+) | | | |
| RIKEN cD 4632401C08 gene | 0.547074 | | | (−) | | | |
| RIKEN cD 4733401N12 gene | | 0.03 | 1.2321 | (+) | | | |
| RIKEN cD 4921528E07 gene | | 0.039 | 1.2027 | (+) | | | |
| RIKEN cD 4921537D05 gene | 1.258399 | | | (+) | | | |
| RIKEN cD 4930506M07 gene | 1.233212 | | | (+) | | | |
| RIKEN cD 4930533K18 gene | 1.325535 | 0.004 | 1.4196 | (+) | | | |
| RIKEN cD 4930542G03 gene | 1.660924 | | | (+) | | | |
| RIKEN cD 4930552N12 gene | 0.625191 | 0.01 | 0.7235 | (−) | | | |
| RIKEN cD 4930579A11 gene | 1.743458 | | | (+) | (+) | RCC | C |
| RIKEN cD 4932442K08 gene | | 0.05 | 1.1747 | (+) | | | |
| RIKEN cD 4933405K01 gene | 1.215798 | | | (+) | | | |
| RIKEN cD 5031412I06 gene | 1.528882 | | | (+) | | | |
| RIKEN cD 5031422I09 gene | 0.71728 | 0.036 | 0.755 | (−) | | | |
| RIKEN cD 5133400A03 gene | 1.242284 | 0.005 | 1.6697 | (+) | | | |
| RIKEN cD 5133401H06 gene | 0.796236 | | | (−) | | | |
| RIKEN cD 5430416A05 gene | 1.253096 | | | (+) | | | |
| RIKEN cD 5630401J11 gene | | 0.002 | 1.4714 | (+) | | | |
| RIKEN cD 5730403B10 gene | 0.817117 | | | (−) | (+) | RCC | DC |
| RIKEN cD 5730406I15 gene | | 0.006 | 1.3059 | (+) | | | |
| RIKEN cD 5730534O06 gene | 0.777482 | | | (−) | | | |
| RIKEN cD 5830445O15 gene | 0.839158 | | | (−) | | | |
| RIKEN cD 6230410I01 gene | | 0.008 | 1.354 | (+) | | | |
| RIKEN cD 6330565B14 gene | 0.484948 | 0.002 | 0.5883 | (−) | | | |
| RIKEN cD 6330583M11 gene | 3.025888 | 8E−04 | 2.0304 | (+) | (+) | RCC | C |
| RIKEN cD 6430559E15 gene | 0.797784 | | | (−) | | | |
| RIKEN cD 6530411B15 gene | 0.748059 | 8E−04 | 0.6185 | (−) | | | |
| RIKEN cD 6720463E02 gene | 1.241163 | | | (+) | | | |
| RIKEN cD 9130011J04 gene | | 0.002 | 1.4288 | (+) | | | |
| RIKEN cD 9130022E05 gene | 0.798272 | | | (−) | | | |
| RIKEN cD 9530058B02 gene | 0.6242 | 0.05 | 0.7595 | (−) | | | |
| RIKEN cD 9530089B04 gene | 0.680734 | 8E−04 | 0.5543 | (−) | | | |
| RIKEN cD A230106A15 gene | 0.855558 | | | (−) | | | |
| RIKEN cD A330103N21 gene | 0.7567217; | | | (−) | | | |
| | 0.700483 | | | | | | |
| RIKEN cD A930008K15 gene | 0.712949 | | | (−) | | | |
| RIKEN cD D630002J15 gene | 0.776514 | | | (−) | | | |
| RIKEN cD E130113K08 gene | | 0.046 | 1.3068 | (+) | | | |
| ring finger protein (C3HC4 type) 19 | | 0.003 | 1.3119 | (+) | | | |
| runt related transcription factor 1 | | 0.012 | 1.3557 | (+) | | | |
| S100 calcium binding protein A10 | 3.102836 | 0.002 | 1.7328 | (+) | | | |
| (calpactin) | | | | | | | |
| S100 calcium binding protein A13 | | 0.033 | 1.2577 | (+) | | | |
| S100 calcium binding protein A4 | 1.715886 | 0.023 | 1.4938 | (+) | | | |
| S100 calcium binding protein A6 | 7.344924 | 8E−04 | 3.3762 | (+) | | | |
| (calcyclin) | | | | | | | |
| S-adenosylhomocysteine hydrolase | | 0.004 | 0.6135 | (−) | (−) | RCC | C |
| SAR1a gene homolog (S. cerevisiae) | 1.167781 | | | (+) | (−) | RCC | DC |
| schlafen 4 | 1.159855 | | | (+) | | | |
| SEC13 related gene (S. cerevisiae) | 1.144426 | | | (+) | | | |
| RIKEN cD 1110003H02 gene | | | | | | | |
| SEC61, gamma subunit (S. cerevisiae) | 1.389586 | | | (+) | (+)/(−) | RCC | conflict |
| secreted acidic cysteine rich | 2.276906 | 0.002 | 2.352 | (+) | (+) | RCC | C |
| glycoprotein | | | | | | | |
| secreted and transmembrane 1 | | 0.033 | 0.7896 | (−) | | | |
| secreted phosphoprotein 1 | 5.051855 | | | (+) | (−)/(+) | RCC | conflict |
| selectin, platelet (p-selectin) ligand | | 0.029 | 1.3367 | (+) | (+) | RCC | C |
| selenium binding protein 2 | | 0.003 | 0.5856 | (−) | (−) | RCC | C |
| selenophosphate synthetase 2 | | 0.014 | 0.7176 | (−) | (−) | RCC | C |
| selenoprotein P, plasma, 1 | 0.591423 | | | (−) | (−) | RCC | C |
| septin 8 | 1.222963 | | | (+) | | | |
| serine (or cysteine) proteise inhibitor, | 1.143231 | | | (+) | | | |
| clade B (ovalbumin), member 2 | | | | | | | |
| serine (or cysteine) proteise inhibitor, | | 8E−04 | 1.808 | (+) | | | |
| clade E (nexin, plasminogen activator | | | | | | | |
| inhibitor type 1), member 2 | | | | | | | |
| serine (or cysteine) proteise inhibitor, | | 9E−04 | 2.3765 | (+) | (+) | RCC | C |
| clade G (C1 inhibitor), member 1 | | | | | | | |
| serine (or cysteine) proteise inhibitor, | 2.222691 | 8E−04 | 1.7609 | (+) | | | |
| clade H (heat shock protein 47), | | | | | | | |
| member 1 | | | | | | | |
| serine hydroxymethyl transferase 1 | | 0.013 | 0.7234 | (−) | (+) | RCC | DC |
| (soluble) | | | | | | | |
| serine hydroxymethyl transferase 2 | 0.700444 | 0.035 | 0.6911 | (−) | (+) | RCC | DC |
| (mitochondrial); RIKEN cD | | | | | | | |
| 2700043D08 gene | | | | | | | |
| serine palmitoyltransferase, long | 0.869628 | | | (−) | (+) | RCC | DC |
| chain base subunit 1 | | | | | | | |
| serine protease inhibitor 6 | | 0.049 | 1.5971 | (+) | | | |
| serine protease inhibitor, Kunitz type 1 | 1.199628 | | | (+) | | | |
| serine protease inhibitor, Kunitz type 2 | 1.224878 | | | (+) | | | |
| serine/arginine repetitive matrix 1 | 1.214449 | | | (+) | | | |
| serine/threonine kise receptor | 1.229013 | | | (+) | | | |
| associated protein | | | | | | | |
| serine/threonine protein kise CISK | 1.188914 | | | (+) | | | |
| serum amyloid A 3 | 2.072529 | | | (+) | | | |
| serum/glucocorticoid regulated kise | | 8E−04 | 0.4203 | (−) | | | |
| serum/glucocorticoid regulated kise 2 | 0.560278 | 0.01 | 0.601 | (−) | | | |
| SET translocation | 1.219476 | | | (+) | (+) | RCC | C |
| sex-lethal interactor homolog | 0.598624 | 8E−04 | 0.4427 | (−) | | | |
| (Drosophila) | | | | | | | |
| SFFV proviral integration 1 | | 0.006 | 1.6359 | (+) | | | |
| SH3 domain binding glutamic acid- | 2.196369 | 8E−04 | 2.0402 | (+) | | | |
| rich protein-like 3 | | | | | | | |
| SH3 domain protein 3 | 1.2681 | | | (+) | | | |
| sideroflexin 1 | 0.866365 | | | (−) | | | |
| sigl sequence receptor, delta | 1.316856 | 0.014 | 1.4178 | (+) | (+) | RCC | C |
| sigl transducer and activator of | | 0.01 | 1.3489 | (+) | (+) | RCC | C |
| transcription 3 | | | | | | | |
| sigling intermediate in Toll pathway- | | 0.002 | 0.7132 | (−) | (−) | RCC | C |
| evolutiorily conserved | | | | | | | |
| single Ig IL-1 receptor related protein | | 0.037 | 0.8027 | (−) | (−) | RCC | C |
| slit homolog 2 (Drosophila) | 0.70698 | | | (−) | | | |
| slit homolog 3 (Drosophila) | | 0.017 | 1.3421 | (+) | | | |
| small inducible cytokine A2 | 2.206498 | 8E−04 | 2.3421 | (+) | | | |
| small inducible cytokine A5 | | 0.003 | 1.7713 | (+) | (+) | RCC | C |
| small inducible cytokine A7 | | 0.019 | 1.4822 | (+) | | | |
| small inducible cytokine A9 | 1.750569 | 0.002 | 1.5855 | (+) | | | |
| small inducible cytokine B subfamily | 2.175863 | 8E−04 | 2.2946 | (+) | | | |
| (Cys-X-Cys), member 10 | | | | | | | |
| small inducible cytokine B subfamily, | | 0.022 | 1.3809 | (+) | | | |
| member 5 | | | | | | | |
| small inducible cytokine subfamily D, 1 | 1.38781 | 0.002 | 1.5826 | (+) | | | |
| small nuclear ribonucleoprotein D2 | 1.387716 | 0.006 | 1.4984 | (+) | (+) | RCC | C |
| small nuclear ribonucleoprotein E | | 8E−04 | 1.4505 | (+) | (+) | RCC | C |
| small nuclear ribonucleoprotein | 1.418612 | 8E−04 | 1.3907 | (+) | | | |
| polypeptide G | | | | | | | |
| small proline-rich protein 1A | | 8E−04 | 2.4047 | (+) | | | |
| SMC (structural maintence of | 1.219049 | | | (+) | (−) | RCC | DC |
| chromosomes 1)-like 1 (S. cerevisiae) | | | | | | | |
| smoothelin | 1.369266 | | | (+) | | | |
| smoothened homolog (Drosophila) | | 0.036 | 0.6399 | (−) | | | |
| soc-2 (suppressor of clear) homolog | | 0.04 | 1.2812 | (+) | | | |
| (C. elegans) | | | | | | | |
| solute carrier family 1, member 1 | | 0.006 | 1.2973 | (+) | (−) | RCC | DC |
| solute carrier family 12, member 1 | 0.278552 | | | (−) | (−) | RCC | C |
| solute carrier family 13 | 1.820774 | 0.001 | 1.5263 | (+) | | | |
| (sodium/sulphate symporters), | | | | | | | |
| member 1 | | | | | | | |
| solute carrier family 13 (sodium- | 0.6572 | 0.041 | 0.6979 | (−) | (−) | RCC | C |
| dependent dicarboxylate transporter), | | | | | | | |
| member 3 | | | | | | | |
| solute carrier family 15 (H+/peptide | 0.639301 | | | (−) | | | |
| transporter), member 2 | | | | | | | |
| solute carrier family 16 | 0.715352 | | | (−) | (−) | RCC | C |
| (monocarboxylic acid transporters), | | | | | | | |
| member 2 | | | | | | | |
| solute carrier family 16 | | 0.009 | 0.6846 | (−) | (+) | RCC | DC |
| (monocarboxylic acid transporters), | | | | | | | |
| member 7 | | | | | | | |
| solute carrier family 2 (facilitated | | 0.047 | 0.6263 | (−) | (−) | RCC | C |
| glucose transporter), member 5 | | | | | | | |
| solute carrier family 22 (organic | | 0.013 | 0.6199 | (−) | (−) | RCC | C |
| anion transporter), member 6 | | | | | | | |
| solute carrier family 22 (organic | 0.404831 | 0.014 | 0.5437 | (−) | (−) | RCC | C |
| anion transporter), member 8/(Roct) | | | | | | | |
| reduced in osteosclerosis transporter | | | | | | | |
| solute carrier family 22 (organic | 0.645465 | 9E−04 | 0.6281 | (−) | (+) | RCC | DC |
| cation transporter), member 1 | | | | | | | |
| solute carrier family 22 (organic | 0.486263 | 0.001 | 0.6191 | (−) | (−)/(+) | RCC | conflict |
| cation transporter), member 1-like | | | | | | | |
| solute carrier family 22 (organic | 0.630304 | 0.004 | 0.6553 | (−) | | | |
| cation transporter), member 2 | | | | | | | |
| solute carrier family 22 (organic | | 0.003 | 0.6747 | (−) | | | |
| cation transporter), member 4 | | | | | | | |
| solute carrier family 22 (organic | 0.513612 | 0.002 | 0.5857 | (−) | | | |
| cation transporter), member 5 | | | | | | | |
| solute carrier family 22 (organic | 0.663072 | | | (−) | | | |
| cation transporter)-like 2 | | | | | | | |
| solute carrier family 25 | 0.616166 | | | (−) | | | |
| (mitochondrial carrier | | | | | | | |
| solute carrier family 25 | | 0.006 | 0.7117 | (−) | | | |
| (mitochondrial carrier | | | | | | | |
| solute carrier family 25 | 0.753628 | | | (−) | | | |
| (mitochondrial deoxynucleotide | | | | | | | |
| carrier), member 19 | | | | | | | |
| solute carrier family 26, member 4 | 0.713201 | 8E−04 | 0.6303 | (−) | | | |
| solute carrier family 27 (fatty acid | 0.586465 | 0.013 | 0.5879 | (−) | | | |
| transporter), member 2 | | | | | | | |
| solute carrier family 3, member 1 | | 0.029 | 0.6994 | (−) | (−) | RCC | C |
| solute carrier family 31, member 1 | 0.850953 | | | (−) | | | |
| solute carrier family 34 (sodium | 0.536109 | | | (−) | | | |
| phosphate), member 1 | | | | | | | |
| solute carrier family 34 (sodium | | 8E−04 | 1.678 | (+) | | | |
| phosphate), member 2 | | | | | | | |
| solute carrier family 35, member A5; | 0.860405 | | | (−) | | | |
| RIKEN cD 1010001J06 gene | | | | | | | |
| solute carrier family 4 (anion | 0.642787 | 0.01 | 0.6624 | (−) | (−) | RCC | C |
| exchanger), member 4 | | | | | | | |
| solute carrier family 6 | 1.136822 | | | (+) | | | |
| (neurotransmitter transporter, | | | | | | | |
| glycine), member 9/glycine | | | | | | | |
| transporter 1 | | | | | | | |
| solute carrier family 7 (cationic | 0.832285 | 0.046 | 0.7065 | (−) | (−) | RCC | C |
| amino acid transporter, y+ system), | | | | | | | |
| member 7 | | | | | | | |
| solute carrier family 7 (cationic | 0.668683 | 8E−04 | 0.6346 | (−) | | | |
| amino acid transporter, y+ system), | | | | | | | |
| member 9 | | | | | | | |
| speckle-type POZ protein | 0.811261 | | | (−) | | | |
| spermatogenesis associated factor | 1.246927 | | | (+) | | | |
| spermidine synthase | 1.524323 | | | (+) | | | |
| spermidine/spermine N1-acetyl | | 0.036 | 1.3351 | (+) | | | |
| transferase | | | | | | | |
| sphingomyelin phosphodiesterase 2, | 0.730054 | | | (−) | | | |
| neutral | | | | | | | |
| splicing factor 3b, subunit 1, 155 kDa | 1.256915 | 0.028 | 1.386 | (+) | (+) | RCC | C |
| splicing factor, arginine/serine-rich 2 | 1.228873 | | | (+) | (+) | RCC | C |
| (SC-35) | | | | | | | |
| split hand/foot deleted gene 1 | | 0.002 | 1.2817 | (+) | (+) | RCC | C |
| src homology 2 domain-containing | 0.826156 | | | (−) | | | |
| transforming protein D | | | | | | | |
| src-like adaptor protein | 1.212423 | | | (+) | | | |
| stearoyl-Coenzyme A desaturase 1 | 0.26606 | 8E−04 | 0.4177 | (−) | | | |
| steroid receptor R activator 1 | 1.155368 | | | (+) | | | |
| sterol carrier protein 2, liver | 0.659454 | 0.039 | 0.6361 | (−) | (+) | RCC | DC |
| striatin, calmodulin binding protein 4/ | | 0.015 | 1.3823 | (+) | | | |
| expressed sequence C80611 | | | | | | | |
| stromal cell derived factor 1 | 0.638758 | | | (−) | | | |
| succinate dehydrogenase complex, | 0.650889 | | | (−) | (−) | RCC | C |
| subunit B, iron sulfur (Ip); RIKEN cD | | | | | | | |
| 0710008N11 gene | | | | | | | |
| succite dehydrogese complex, subunit | 0.63565 | | | (−) | | | |
| A, flavoprotein (Fp) | | | | | | | |
| succite-Coenzyme A ligase, ADP- | 0.738104 | | | (−) | | | |
| forming, beta subunit | | | | | | | |
| succite-Coenzyme A ligase, GDP- | 0.8423 | | | (−) | | | |
| forming, beta subunit | | | | | | | |
| sulfotransferase-related protein | | 0.017 | 1.2358 | (+) | | | |
| SULT-X1 | | | | | | | |
| superoxide dismutase 2, | 0.627202 | 0.023 | 0.6795 | (−) | (+) | RCC | DC |
| mitochondrial | | | | | | | |
| surfeit gene 4 | 1.173262 | | | (+) | (+) | RCC | C |
| SWI/SNF related, matrix associated, | 1.34736; | | | (+) | (+) | RCC | C |
| actin dependent regulator of | 1.192875 | | | | | | |
| chromatin, subfamily a, member 5 | | | | | | | |
| SWI/SNF related, matrix associated, | 1.375898 | | | (+) | (+) | RCC | C |
| actin dependent regulator of | | | | | | | |
| chromatin, subfamily e, member 1 | | | | | | | |
| syndecan 1 | 1.755052 | | | (+) | (−) | RCC | DC |
| syntrophin, basic 2 | 1.145842 | | | (+) | | | |
| TAF10 R polymerase II, TATA box | 1.437509 | | | (+) | | | |
| binding protein (TBP)-associated | | | | | | | |
| factor, 30 kDa | | | | | | | |
| TAF9 R polymerase II, TATA box | 1.315523 | | | (+) | | | |
| binding protein (TBP)-associated | | | | | | | |
| factor, 32 kDa | | | | | | | |
| talin 2 | 0.590195 | 8E−04 | 0.5429 | (−) | | | |
| TATA box binding protein-like | | 0.007 | 1.336 | (+) | | | |
| protein | | | | | | | |
| T-box 6 | 1.613638 | 8E−04 | 1.8123 | (+) | | | |
| T-cell specific GTPase | | 0.003 | 2.029 | (+) | | | |
| T-cell, immune regulator 1 | | 9E−04 | 1.3678 | (+) | | | |
| TEA domain family member 2 | 1.218905 | | | (+) | | | |
| tescin C | 2.161393 | 8E−04 | 2.1224 | (+) | | | |
| tescin XB | 0.81373 | | | (−) | | | |
| testis derived transcript | 1.466866 | | | (+) | (+) | RCC | C |
| tetranectin (plasminogen binding | 0.69379 | | | (−) | | | |
| protien) | | | | | | | |
| tetratricopeptide repeat domain | | 0.032 | 1.3798 | (+) | (+) | RCC | C |
| TG interacting factor | 1.49248 | 8E−04 | 1.6651 | (+) | (+) | RCC | C |
| thiamin pyrophosphokise | 0.815518 | | | (−) | | | |
| thioesterase, adipose associated | 0.608099 | 8E−04 | 0.4926 | (−) | | | |
| thioether S-methyltransferase | | 0.002 | 0.4638 | (−) | | | |
| thioredoxin 1 | 1.547693 | 0.025 | 1.52 | (+) | (−)/(+) | RCC | conflict |
| thioredoxin 2 | | 0.006 | 0.7742 | (−) | | | |
| thioredoxin-like (32 kD) | 1.285715 | | | (+) | | | |
| thrombospondin 1 | | 0.003 | 1.7297 | (+) | (−) | RCC | DC |
| thymidine kise 1 | 1.822689 | | | (+) | (+) | RCC | C |
| thymoma viral proto-oncogene 1 | 1.502028 | | | (+) | (+) | RCC | C |
| thymosin, beta 4, X chromosome | 2.365009 | 8E−04 | 2.6847 | (+) | (+) | | C |
| thyroid hormone responsive SPOT14 | 0.293263 | 8E−04 | 0.4343 | (−) | | | |
| homolog (Rattus) | | | | | | | |
| Tial1 cytotoxic granule-associated R | 1.21967 | | | (+) | (+) | RCC | C |
| binding protein-like 1 | | | | | | | |
| tight junction protein 2 | | 0.015 | 1.4429 | (+) | (−) | RCC | DC |
| tissue inhibitor of metalloproteise | 2.944279 | 8E−04 | 2.854 | (+) | (+) | RCC | C |
| Tnf receptor-associated factor 2 | 1.31305 | | | (+) | | | |
| toll-like receptor 2 | | 0.014 | 1.4711 | (+) | | | |
| topoisomerase (D) III beta | 0.840401 | | | (−) | (+) | RCC | DC |
| TRAF-interacting protein | 1.192268 | | | (+) | | | |
| transcobalamin 2 | 0.522163 | 8E−04 | 0.5031 | (−) | (−) | RCC | C |
| transcription elongation factor A | 0.789024 | | | (−) | | | |
| (SII), 3 | | | | | | | |
| transcription elongation regulator 1 | 5.521204 | 8E−04 | 3.3877 | (+) | | | |
| (CA150) | | | | | | | |
| transcription factor 21 | | 8E−04 | 1.7517 | (+) | (−) | RCC | DC |
| transcription factor 4 | | 0.016 | 1.3902 | (+) | | | |
| transcription factor Dp 1 | | 0.003 | 1.3295 | (+) | (+) | RCC | C |
| transformation related protein 53 | 1.362828 | | | (+) | (+)/(−??) | RCC | conflict |
| transformed mouse 3T3 cell double | | 0.044 | 1.3109 | (+) | (+) | RCC | C |
| minute 2 | | | | | | | |
| transforming growth factor beta 1 | 2.395573 | 0.008 | 1.5674 | (+) | (+) | RCC | C |
| induced transcript 4 | | | | | | | |
| transforming growth factor, beta | 2.085258 | 8E−04 | 1.8572 | (+) | (+) | RCC | C |
| induced, 68 kDa | | | | | | | |
| transgelin | 1.600162 | 8E−04 | 2.5038 | (+) | | | |
| translin | 1.191429 | | | (+) | | | |
| transmembrane 7 superfamily | 0.786219 | | | (−) | | | |
| member 1 | | | | | | | |
| transmembrane protein 8 (five | 0.7753253; | 0.023 | 0.6612 | (−) | | | |
| membrane-spanning domains) | 0.7539193 | | | | | | |
| Trans-prenyltransferase | | 0.003 | 1.3624 | (+) | | | |
| transthyretin | 0.592428 | | | (−) | | | |
| trinucleotide repeat containing 11 | | 0.028 | 1.3829 | (+) | | | |
| (THR-associated protein, 230 kDa | | | | | | | |
| subunit) | | | | | | | |
| tropomyosin 2, beta | 1.834774 | | | (+) | | | |
| tropomyosin 3, gamma | 2.00637 | 8E−04 | 1.5813 | (+) | | | |
| tubulin alpha 1 | | 8E−04 | 2.2002 | (+) | | | |
| tubulin alpha 2 | 2.656871 | 0.002 | 2.0093 | (+) | | | |
| tubulin, beta 5 | 3.080405 | | | (+) | (+) | RCC | C |
| tuftelin 1 | 1.497479 | | | (+) | | | |
| tumor necrosis factor receptor | 1.355122 | | | (+) | | | |
| superfamily, member 10b | | | | | | | |
| tumor necrosis factor receptor | 1.431735 | 0.021 | 1.3333 | (+) | (+) | RCC | C |
| superfamily, member 1a | | | | | | | |
| tumor necrosis factor receptor | | 0.024 | 1.3824 | (+) | | | |
| superfamily, member 1b | | | | | | | |
| tumor protein p53 binding protein, 2/ | | 0.01 | 0.6437 | (−) | | | |
| expressed sequence AI746547 | | | | | | | |
| tumor rejection antigen gp96 | 1.322746 | | | (+) | (+) | RCC | C |
| tumor-associated calcium sigl | 2.166496 | 0.002 | 1.6128 | (+) | (−) | RCC | DC |
| transducer 2 | | | | | | | |
| tural killer tumor recognition | 1.678022 | 8E−04 | 2.0726 | (+) | | | |
| sequence | | | | | | | |
| TYRO protein tyrosine kise binding | 1.850489 | 8E−04 | 2.1288 | (+) | (+) | RCC | C |
| protien | | | | | | | |
| tyrosine 3-monooxygese/tryptophan | 1.374164 | | | (+) | | | |
| 5-monooxygese activation protein, | | | | | | | |
| epsilon polypeptide | | | | | | | |
| tyrosine 3-monooxygese/tryptophan | 1.598302 | 0.005 | 1.5449 | (+) | (+) | RCC | C |
| 5-monooxygese activation protein, eta | | | | | | | |
| polypeptide | | | | | | | |
| ubiquitin specific protease 2 | 0.387442 | 8E−04 | 0.4121 | (−) | (−) | RCC | C |
| ubiquitin specific protease 7 | 1.368404 | | | (+) | | | |
| (expressed sequence AA409944) | | | | | | | |
| ubiquitin-conjugating enzyme E2D 2 | | 0.009 | 1.3738 | (+) | | | |
| ubiquitin-conjugating enzyme E2H | 1.73032 | 0.002 | 1.6531 | (+) | (+) | RCC | C |
| ubiquitin-conjugating enzyme E2I | 1.501533 | | | (+) | | | |
| ubiquitin-conjugating enzyme E2L 3 | 1.276359 | | | (+) | | | |
| ubiquitin-conjugating enzyme E2N | 1.253604 | 0.008 | 1.3224 | (+) | | | |
| ubiquitin-like 1 | 1.235698 | | | (+) | (+) | RCC | C |
| ubiquitin-like 1 (sentrin) activating | 1.209625 | | | (+) | (+) | RCC | C |
| enzyme E1A | | | | | | | |
| ubiquitin-like 1 (sentrin) activating | 1.319403 | | | (+) | | | |
| enzyme E1B | | | | | | | |
| UDP-Gal:betaGlcc beta 1,3- | 0.790361 | | | (−) | | | |
| galactosyltransferase, polypeptide 3 | | | | | | | |
| UDP-Gal:betaGlcc beta 1,4- | 1.226956 | | | (+) | | | |
| galactosyltransferase, polypeptide 2 | | | | | | | |
| UDP-N-acetyl-alpha-D- | 1.374851 | 0.031 | 1.4925 | (+) | | | |
| galactosamine:(N-acetylneuraminyl)- | | | | | | | |
| galactosylglucosylceramide-beta-1,4- | | | | | | | |
| N-acetylgalactosaminyltransferase | | | | | | | |
| Unknown | 1.631964 | 8E−04 | 1.8313 | (+) | | | |
| Unknown | 1.452741 | 0.012 | 1.5847 | (+) | | | |
| Unknown | 1.622317 | 0.001 | 1.369 | (+) | | | |
| Unknown | 0.196028 | 0.019 | 0.4352 | (−) | | | |
| Unknown | 1.599236; | 0.0008; | 1.871876; | (+) | | | |
| | 1.758187 | 0.0008 | 2.313198 | | | | |
| Unknown | 1.288468 | 8E−04 | 1.4377 | (+) | | | |
| Unknown | 0.665629 | 0.013 | 0.6782 | (−) | | | |
| Unknown | 1.361226 | 0.003 | 1.4285 | (+) | | | |
| Unknown | 1.196485 | 9E−04 | 1.556 | (+) | | | |
| Unknown | 1.555723 | 8E−04 | 1.9514 | (+) | | | |
| Unknown | 0.42673 | | | (−) | | | |
| Unknown | 1.666878 | | | (+) | | | |
| Unknown | 0.801886 | | | (−) | | | |
| Unknown | 0.724904 | | | (−) | | | |
| Unknown | 1.291594 | | | (+) | | | |
| Unknown | 0.84103 | | | (−) | | | |
| Unknown | 1.577602 | | | (+) | | | |
| Unknown | 0.695732 | | | (−) | | | |
| Unknown | 0.863638 | | | (−) | | | |
| Unknown | 0.648175 | | | (−) | | | |
| Unknown | 0.802178 | | | (−) | | | |
| Unknown | 0.740476 | | | (−) | | | |
| Unknown | 0.700466 | | | (−) | | | |
| Unknown | 1.210575 | | | (+) | | | |
| Unknown | 1.350042 | | | (+) | | | |
| Unknown | | 0.009 | 0.6237 | (−) | | | |
| Unknown | | 0.015 | 1.4949 | (+) | | | |
| Unknown | | 0.012 | 0.7258 | (−) | | | |
| Unknown | | 0.002 | 1.5282 | (+) | | | |
| Unknown | | 0.023 | 0.6626 | (−) | | | |
| Unknown | | 0.013 | 0.789 | (−) | | | |
| Unknown | | 0.006 | 0.6713 | (−) | | | |
| Unknown | | 0.002 | 1.2986 | (+) | | | |
| Unknown | | 8E−04 | 4.6753 | (+) | | | |
| upregulated during skeletal muscle | | 8E−04 | 0.5704 | (−) | | | |
| growth 5 | | | | | | | |
| upstream transcription factor 1 | 0.739612 | | | (−) | | | |
| urokise plasminogen activator | 1.496585 | 0.004 | 1.3851 | (+) | (+) | RCC | C |
| receptor | | | | | | | |
| UUDP glycosyltransferase 1 family, | | 8E−04 | 0.5626 | (−) | | | |
| polypeptide A6 | | | | | | | |
| vascular cell adhesion molecule 1 | | 8E−04 | 3.207 | (+) | (+) | RCC | C |
| vascular endothelial growth factor A | 0.798289 | 0.005 | 0.8443 | (−) | (+) | RCC | DC |
| vascular endothelial zinc finger 1; | 0.923209 | | | (−) | | | |
| expressed sequence AI848691 | | | | | | | |
| vasodilator-stimulated | 1.377774 | 0.001 | 1.7852 | (+) | | | |
| phosphoprotein | | | | | | | |
| vitamin D receptor | 0.636449 | | | (−) | | | |
| v-ral simian leukemia viral oncogene | | 0.043 | 1.3333 | (+) | (+) | RCC | C |
| homolog A (ras related) | | | | | | | |
| v-ral simian leukemia viral oncogene | 1.70831 | 8E−04 | 1.5091 | (+) | | | |
| homolog B (ras related) | | | | | | | |
| WD repeat domain 1 | 1.622447 | | | (+) | | | |
| Williams-Beuren syndrome | 0.698155 | | | (−) | (−) | RCC | C |
| chromosome region 14 homolog | | | | | | | |
| (human) | | | | | | | |
| WNT1 inducible sigling pathway | | 0.003 | 1.3413 | (+) | | | |
| protein 1 | | | | | | | |
| X (ictive)-specific transcript, | | 8E−04 | 1.5 | (+) | | | |
| antisense | | | | | | | |
| X transporter protein 2 | | 0.038 | 0.7554 | (−) | | | |
| Yamaguchi sarcoma viral (v-yes) | | 0.03 | 1.2634 | (+) | | | |
| oncogene homolog | | | | | | | |
| Yamaguchi sarcoma viral (v-yes-1) | | 0.005 | 1.4026 | (+) | (+) | RCC | C |
| oncogene homolog | | | | | | | |
| yolk sac gene 2 | 0.791519 | | | (−) | | | |
| zinc finger like protein 1 | | 0.05 | 0.6885 | (−) | | | |
| zinc finger protein 144 | | 0.004 | 1.5968 | (+) | (−) | RCC | DC |
| zinc finger protein 36, C3H type-like 1 | 1.775831 | 0.001 | 1.6203 | (+) | (+) | RCC | C |
| zinc finger protein 36, C3H type-like 2 | 2.031905 | 0.019 | 1.4281 | (+) | | | |
| zuotin related factor 2 | 1.298786 | | | (+) | | | |

[symbol]

indicates data missing or illegible when filed

TABLE 16
An ontology analysis in timely dependent fashion: distinct and common ontologies.
The genes in the three phases of renal regeneration and the concordant and discordant genes
are analyzed for GO (summary sheets). These genes were crossed with the data from
supplemental Table 4 (cross sheets); green down-regulated and red up-regulated in RRR.

| Gene Category | Up | Down | Genes |
|---|---|---|---|
| cytosolic ribosome (sensu | 12 | 0 | RPL29, RPL36A, RPL5, RPL6, SYN1, RPS16, RPS3A, RPS4X, RPS6, |
| Eukarya) | | | RPS7, RPS23, RPL38 |
| carboxylic acid | 3 | 24 | TNFRSF1A, CTPS, ELOVL1, AUH, CPT1A, FAH, FOLR1, GLUL, GPAT, |
| metabolism | | | HADHSC, HPD, LPL, ME1, PAH, PKLR, PRODH, SCD, SCP2, SLC7A7, |
| | | | SLC27A2, MLYCD, ACADSB, GATM, CRYL1, CACH-1, MTHFD1, |
| | | | MGC37818 |
| organic acid metabolism | 3 | 24 | TNFRSF1A, CTPS, ELOVL1, AUH, CPT1A, FAH, FOLR1, GLUL, GPAT, |
| | | | HADHSC, HPD, LPL, ME1, PAH, PKLR, PRODH, SCD, SCP2, SLC7A7, |
| | | | SLC27A2, MLYCD, ACADSB, GATM, CRYL1, CACH-1, MTHFD1, |
| | | | MGC37818 |
| structural constituent of | 20 | 0 | GADD45A, LAMR1, PTMA, RPL10A, RPL29, RPL36A, RPL5, RPL6, |
| ribosome | | | SYN1, RPS16, RPS3A, RPS4X, RPS6, RPS7, RPL27A, RPL3, RPLP1, |
| | | | RPS23, RPL35, RPL38 |
| ribosome | 21 | 0 | GADD45A, LAMR1, PTMA, RPL10A, RPL29, RPL36A, RPL5, RPL6, |
| | | | SYN1, RPS16, RPS3A, RPS4X, RPS6, RPS7, RPL27A, RPL3, CTPS, |
| | | | RPLP1, RPS23, RPL35, RPL38 |
| structural molecule activity | 36 | 0 | ACTB, ACTG2, ACTG1, ACTA2, CLDN1, CLDN4, COL4A1, COL5A2, |
| | | | CRYM, GADD45A, EMP3, FBN1, KRT8, LAMR1, PTMA, RPL10A, |
| | | | RPL29, RPL36A, RPL5, RPL6, SYN1, RPS16, RPS3A, RPS4X, RPS6, |
| | | | RPS7, TUBA2, RPL27A, RPL3, CLDN7, RPLP1, BAF53A, EFEMP2, |
| | | | RPS23, RPL35, RPL38 |
| fatty acid metabolism | 2 | 12 | TNFRSF1A, ELOVL1, CPT1A, GPAT, HADHSC, LPL, PKLR, SCD, SCP2, |
| | | | SLC27A2, MLYCD, ACADSB, CRYL1, CACH-1 |
| ribonucleoprotein complex | 25 | 0 | GADD45A, HNRPA1, LAMR1, PTMA, RPL10A, RPL29, RPL36A, RPL5, |
| | | | RPL6, SYN1, RPS16, RPS3A, RPS4X, RPS6, RPS7, RPL27A, RPL3, CTPS, |
| | | | RPLP1, RPS23, RPL35, RPL38, SNRPG, SF3B1, SNRPD2 |
| ribosome biogenesis | 10 | 0 | RPL29, RPL36A, RPL5, RPL6, SYN1, RPS16, RPS3A, RPS4X, RPS6, RPS7 |
| ribosome biogenesis and | 10 | 0 | RPL29, RPL36A, RPL5, RPL6, SYN1, RPS16, RPS3A, RPS4X, RPS6, RPS7 |
| assembly | | | |
| oxidoreductase activity | 7 | 23 | AKR1B10, TXN, YWHAH, GMPR, H3f3b, ABP1, DIA1, BCKDHA, |
| | | | CYP2A13, CYP2D6, CYP2J2, DIO1, HADHSC, HPD, ME1, MDH1, NNT, |
| | | | PAH, PRODH, SCD, SOD2, AASS, IVD, ACADSB, CRYL1, DMGDH, |
| | | | ADH8, 0610025I19Rik, MTHFD1, ALDH7A1 |
| cytoplasm organization | 23 | 2 | ACTB, ACTG2, ACTG1, ACTA2, CAPZB, CDC42, CNN2, KRT8, LSP1, |
| and biogenesis | | | TMSB4X, RPL29, RPL36A, RPL5, RPL6, SYN1, RPS16, RPS3A, RPS4X, |
| | | | RPS6, RPS7, TAGLN, TUBA2, CORO1B, ABCD3, SCP2 |
| cytosol | 15 | 6 | MT1A, PSME1, RPL29, RPL36A, RPL5, RPL6, SYN1, RPS16, RPS3A, |
| | | | RPS4X, RPS6, RPS7, RPS23, BZW2, RPL38, INPP5B, ME1, MDH1, PKLR, |
| | | | FRAP1, CACH-1 |
| amino acid catabolism | 0 | 6 | AUH, FAH, HPD, PAH, PRODH, MGC37818 |
| aromatic compound | 2 | 6 | CTPS, DKFZP434P106, FAH, FOLR1, HPD, PAH, 2010012D11Rik, |
| metabolism | | | MTHFD1 |
| amine catabolism | 0 | 6 | AUH, FAH, HPD, PAH, PRODH, MGC37818 |
| extracellular space | 49 | 23 | ADAM12, BGN, BST1, C1QA, C3, SERPINH1, CD24, CD68, CDH3, |
| | | | CLDN1, CLDN4, COL4A1, COL5A2, CTSS, EDN1, EMP3, F2RL1, F3, |
| | | | FBN1, FCER1G, FCGR3A, AKR1B10, GALGT, Gp49a, Gp49b, SCYB10, |
| | | | CYR61, LY6E, MGP, NPDC1, FXYD5, OSMR, PLAUR, PTPRC, SCYA2, |
| | | | CCL9, SPARC, TGFBI, TIMP1, TNC, TNFRSF1A, TYROBP, PLAB, AXL, |
| | | | CLDN7, SLC13A1, PF4, TACSTD2, ABP1, BCKDHA, CYP2J2, DIO1, |
| | | | DNASE1, DPEP1, EGF, F13B, FOLR1, NAP1, KL, Klk1/6, LPL, MEP1A, |
| | | | SLC22A1L, ENPP2, ABCD3, TCN2, VEGF, SLC27A2, TMEM8, |
| | | | DKFZp564K1964.1, CES3, SLC13A3 |
| eukaryotic 43S | 5 | 0 | EIF3S6, RPS4X, RPS6, RPS7, RPS23 |
| preinitiation complex | | | |
| physiological process | 134 | 88 | ACTB, ACTG2, ACTG1, ACTA2, ADAM12, ADAMTS1, ADSS, ANXA5, |
| | | | ANXA6, ARHB, ARHC, BCL2A1, ARPC2, BST1, ZFP36L1, ZFP36L2, |
| | | | C1QA, C3, CAPZB, SERPINH1, CD24, CD68, CD72, CDC42, SOCS3, |
| | | | CLDN4, CCR2, CNN2, COL5A2, CTSS, GADD45A, EDN1, EIF4EBP1, |
| | | | ELF3, EMP3, F2RL1, F3, FBN1, FCER1G, FCGR3A, AKR1B10, GALGT, |
| | | | GNAI2, GNB2L1, H2-D1, PTPN6, HMGN2, HMGB3, HNRPA1, ICAM1, |
| | | | SCYB10, CYR61, EIF3S6, KRT8, LAMR1, LSP1, LY6E, MGP, MT1A, |
| | | | MYC, BIRC1, NKTR, NPDC1, NPM1, FXYD5, PLAUR, PSME1, PTMA, |
| | | | TMSB4X, PTPRC, RBM3, RPL10A, RPL29, RPL36A, RPL5, RPL6, SYN1, |
| | | | RPS16, RPS3A, RPS4X, RPS6, RPS7, S100A6, SCYA2, CCL9, SCYD1, |
| | | | SPARC, SSR4, TAGLN, TBX6, TSC22, TGFBI, TGIF, TNFRSF1A, |
| | | | TUBA2, TXN, TYROBP, UBE2H, YWHAH, CORO1B, CFDP1, COPEB, |
| | | | AXL, RPL27A, RPL3, CLIC4, H2AFZ, CTPS, ELOVL1, SLC13A1, RPLP1, |
| | | | TCERG1, PTPN9, CSDA, BAF53A, ELF4, PF4, TACSTD2, PMAIP1, |
| | | | EFEMP2, GMPR, RPS23, RPL35, H3f3b, BZW2, RPL38, SNRPG, |
| | | | DKFZP434P106, ABP1, SF3B1, UBE2N, SNRPD2, DIA1, CLIC1, Ak4, |
| | | | AUH, BCKDHA, CALB1, CPT1A, CYP2A13, CYP2D6, CYP2J2, DIO1, |
| | | | DNASE1, DPEP1, EGF, F13B, FAH, FOLR1, G6PC, GAS2, GGT1, GLUL, |
| | | | GPAT, GK, HADHSC, HPD, HPN, INPP5B, NAP1, KHK, KL, BTEB1, |
| | | | Klk1/6, Klk26, LPL, MEP1A, ME1, MDH1, MUT, NNT, SLC22A1L, PAH, |
| | | | ENPP2, PKLR, PAPOLA, HLF, PRODH, ABCD3, SLC22A8, SCD, SCP2, |
| | | | SLC22A1, SLC22A2, SLC22A5, SLC7A7, SOD2, TCN2, THRSP, VEGF, |
| | | | SLC26A4, SLC27A2, RPC5, SGK2, JDP1, AASS, SLC7A9, USP2, SLC4A4, |
| | | | PGAM2, IVD, MLYCD, FRAP1, HERPUD1, OSBPL1A, KLF15, FLJ10241, |
| | | | ACADSB, GATM, FLJ13448, 2010012D11Rik, MGC15416, CRYL1, |
| | | | DMGDH, CACH-1, ADH8, 0610025I19Rik, SLC17A3, MTHFD1, |
| | | | ALDH7A1, SLC13A3, MGC37818 |
| blood coagulation | 6 | 2 | ANXA5, ANXA6, F2RL1, F3, PF4, EFEMP2, F13B, MGC15416 |
| response to external | 30 | 6 | ACTG1, BST1, C1QA, C3, SERPINH1, CD24, CD72, CCR2, FBN1, |
| stimulus | | | FCER1G, FCGR3A, GNAI2, H2-D1, ICAM1, SCYB10, CYR61, LSP1, |
| | | | LY6E, PSME1, PTMA, PTPRC, SCYA2, CCL9, SCYD1, TNFRSF1A, |
| | | | TYROBP, COPEB, PF4, TACSTD2, ABP1, SLC22A1L, SOD2, SLC26A4, |
| | | | HERPUD1, OSBPL1A, ALDH7A1 |
| eukaryotic 48S initiation | 4 | 0 | RPS4X, RPS6, RPS7, RPS23 |
| complex | | | |
| cytosolic small ribosomal | 4 | 0 | RPS4X, RPS6, RPS7, RPS23 |
| subunit (sensu Eukarya) | | | |
| hemostasis | 6 | 2 | ANXA5, ANXA6, F2RL1, F3, PF4, EFEMP2, F13B, MGC15416 |
| extracellular | 54 | 23 | ADAM12, ADAMTS1, BGN, BST1, C1QA, C3, SERPINH1, CD24, CD68, |
| | | | CDH3, CLDN1, CLDN4, COL4A1, COL5A2, CSTB, CTSS, EDN1, EMP3, |
| | | | F2RL1, F3, FBN1, FCER1G, FCGR3A, AKR1B10, GALGT, Gp49a, Gp49b, |
| | | | SCYB10, CYR61, LY6E, MGP, NPDC1, FXYD5, OSMR, PLAUR, PTPRC, |
| | | | SCYA2, CCL9, SCYD1, SPARC, TGFBI, TIMP1, TNC, TNFRSF1A, |
| | | | TYROBP, CFDP1, PLAB, AXL, CLDN7, SLC13A1, PF4, TACSTD2, |
| | | | EFEMP2, ABP1, BCKDHA, CYP2J2, DIO1, DNASE1, DPEP1, EGF, F13B, |
| | | | FOLR1, NAP1, KL, Klk1/6, LPL, MEP1A, SLC22A1L, ENPP2, ABCD3, |
| | | | TCN2, VEGF, SLC27A2, TMEM8, DKFZp564K1964.1, CES3, SLC13A3 |
| biosynthesis | 24 | 11 | ADSS, GADD45A, EIF4EBP1, EIF3S6, LAMR1, RPL10A, RPL29, |
| | | | RPL36A, RPL5, RPL6, RPS16, RPS3A, RPS4X, RPS6, RPS7, RPL27A, |
| | | | RPL3, CTPS, ELOVL1, RPLP1, RPS23, RPL35, BZW2, RPL38, G6PC, |
| | | | GGT1, GLUL, GPAT, PAH, PKLR, PRODH, SCD, MLYCD, GATM, |
| | | | MTHFD1 |
| cell organization and | 26 | 2 | ACTB, ACTG2, ACTG1, ACTA2, CAPZB, CDC42, CNN2, KRT8, LSP1, |
| biogenesis | | | TMSB4X, RPL29, RPL36A, RPL5, RPL6, SYN1, RPS16, RPS3A, RPS4X, |
| | | | RPS6, RPS7, TAGLN, TUBA2, CORO1B, CFDP1, H2AFZ, BAF53A, |
| | | | ABCD3, SCP2 |
| response to abiotic | 12 | 4 | ACTG1, SERPINH1, CCR2, FBN1, GNAI2, SCYB10, CYR61, LSP1, |
| stimulus | | | SCYA2, CCL9, PF4, ABP1, SLC22A1L, SLC26A4, OSBPL1A, ALDH7A1 |
| protein biosynthesis | 21 | 0 | GADD45A, EIF4EBP1, EIF3S6, LAMR1, RPL10A, RPL29, RPL36A, RPL5, |
| | | | RPL6, RPS16, RPS3A, RPS4X, RPS6, RPS7, RPL27A, RPL3, RPLP1, |
| | | | RPS23, RPL35, BZW2, RPL38 |
| actin binding | 8 | 3 | CAPZB, CNN2, LSP1, TMSB4X, TAGLN, VASP, CORO1B, TPM3, |
| | | | DNASE1, TLN2, SLC13A3 |
| posttranslational | 4 | 3 | BST1, CD24, LY6E, PLAUR, DPEP1, FOLR1, LPL |
| membrane targeting | | | |
| macromolecule | 24 | 6 | ADSS, GADD45A, EIF4EBP1, EIF3S6, LAMR1, RPL10A, RPL29, |
| biosynthesis | | | RPL36A, RPL5, RPL6, RPS16, RPS3A, RPS4X, RPS6, RPS7, RPL27A, |
| | | | RPL3, CTPS, ELOVL1, RPLP1, RPS23, RPL35, BZW2, RPL38, G6PC, |
| | | | GPAT, PKLR, SCD, MLYCD, MTHFD1 |
| small ribosomal subunit | 5 | 0 | LAMR1, RPS4X, RPS6, RPS7, RPS23 |
| L-phenylalanine | 0 | 3 | FAH, HPD, PAH |
| metabolism | | | |
| phenylalanine catabolism | 0 | 3 | FAH, HPD, PAH |
| RNA binding | 17 | 2 | HNRPA1, NPM1, RBM3, RPL5, RPS16, RPS3A, RPS4X, RPS6, RPS7, |
| | | | RPL27A, RPL3, RPLP1, RPS23, RPL38, SNRPG, SF3B1, SNRPD2, AUH, |
| | | | PAPOLA |
| mitochondrion | 3 | 22 | CLIC4, PMAIP1, H3f3b, Ak4, AUH, BCKDHA, CPT1A, GLUL, GPAT, |
| | | | GK, HADHSC, KHK, MUT, NNT, PRODH, SCP2, SOD2, IVD, MLYCD, |
| | | | FLJ10241, ACADSB, GATM, FLJ13448, DMGDH, 0610025I19Rik |
| amino acid and derivative | 1 | 11 | CTPS, AUH, DIO1, FAH, GLUL, HPD, PAH, PRODH, SLC7A7, GATM, |
| metabolism | | | MTHFD1, MGC37818 |
| response to chemical | 9 | 1 | CCR2, GNAI2, SCYB10, CYR61, LSP1, SCYA2, CCL9, PF4, ABP1, |
| substance | | | SLC22A1L |
| anion transporter activity | 1 | 4 | SLC13A1, SLC22A1L, SLC26A4, SLC4A4, SLC13A3 |
| aromatic amino acid | 0 | 3 | FAH, HPD, PAH |
| family catabolism | | | |
| aromatic compound | 0 | 3 | FAH, HPD, PAH |
| catabolism | | | |
| amino acid metabolism | 1 | 9 | CTPS, AUH, FAH, GLUL, HPD, PAH, PRODH, SLC7A7, MTHFD1, |
| | | | MGC37818 |
| protein-ER targeting | 4 | 3 | BST1, CD24, LY6E, PLAUR, DPEP1, FOLR1, LPL |
| anion transport | 3 | 4 | CLIC4, SLC13A1, CLIC1, SLC22A1L, SLC26A4, SLC4A4, SLC13A3 |
| protein-membrane | 4 | 3 | BST1, CD24, LY6E, PLAUR, DPEP1, FOLR1, LPL |
| targeting | | | |
| inorganic anion transport | 3 | 2 | CLIC4, SLC13A1, CLIC1, SLC26A4, SLC4A4 |
| response to biotic | 24 | 2 | BST1, C1QA, C3, CD24, CD72, CCR2, FCER1G, FCGR3A, H2-D1, |
| stimulus | | | ICAM1, SCYB10, LSP1, LY6E, PSME1, PTMA, PTPRC, SCYA2, CCL9, |
| | | | SCYD1, TNFRSF1A, TYROBP, COPEB, PF4, TACSTD2, SOD2, |
| | | | HERPUD1 |
| actin filament | 3 | 1 | ACTG2, ACTG1, BAF53A, GAS2 |
| immunoglobulin binding | 3 | 0 | FCER1G, FCGR3A, LGALS3 |
| ion transporter activity | 2 | 10 | SLC13A1, H3f3b, NNT, SLC22A1L, SLC22A8, SLC22A1, SLC22A2, |
| | | | SLC22A5, TCN2, SLC26A4, SLC4A4, SLC13A3 |
| chemotaxis | 7 | 0 | CCR2, SCYB10, CYR61, LSP1, SCYA2, CCL9, PF4 |
| taxis | 7 | 0 | CCR2, SCYB10, CYR61, LSP1, SCYA2, CCL9, PF4 |
| defense response | 24 | 0 | BST1, C1QA, C3, CD24, CD72, CCR2, FCER1G, FCGR3A, H2-D1, |
| | | | ICAM1, SCYB10, LSP1, LY6E, PSME1, PTMA, PTPRC, SCYA2, CCL9, |
| | | | SCYD1, TNFRSF1A, TYROBP, COPEB, PF4, TACSTD2 |
| chemokine receptor | 5 | 0 | SCYB10, SCYA2, CCL9, SCYD1, PF4 |
| binding | | | |
| G-protein-coupled | 5 | 0 | SCYB10, SCYA2, CCL9, SCYD1, PF4 |
| receptor binding | | | |
| chemokine activity | 5 | 0 | SCYB10, SCYA2, CCL9, SCYD1, PF4 |
| heparin binding | 4 | 2 | ADAMTS1, CYR61, PF4, ABP1, LPL, VEGF |
| amine metabolism | 1 | 11 | CTPS, AUH, DIO1, FAH, GLUL, HPD, PAH, PRODH, SLC7A7, GATM, |
| | | | MTHFD1, MGC37818 |

TABLE 17
The differently expressed genes in both RRR and RCC exhibited distinct
ontologies for the concordance vs. discordance genes. The differentially expressed genes in
both RRR and RCC were clustered according to their concordance vs. discordant change.
Functional ontology was analysis performed (p < 0.05). The ontologies are hyperlinked to
EMBL-EBI. The average RRR expression of each ontology is presented in a green to red
scale; green down-regulated, red up-regulated. The number and average RRR expression of
genes up-/down-regulated in both RRR and RCC, the category p-value and enrichment are
also given (the expression direction and values is as in RRR relative to the normal kidney).

| | Concordant | | | | | |
|---|---|---|---|---|---|---|
| | | Total | | Total | No | |
| | Average | Expression | No Genes- | Expression | Genes- | |
| Category | Expression | UP | UP | DOWN | DOWN | p < 0.05 |
| immunoglobulin binding | 1.103 | 3.3092367 | 3 | 0 | 0 | 0.0340422 |
| extracellular matrix structural | 0.884 | 4.4205293 | 5 | 0 | 0 | 0.0140517 |
| constituent conferring tensile | | | | | | |
| strength | | | | | | |
| structural constituent of ribosome | 0.741 | 17.785127 | 24 | 0 | 0 | 4.242E−10 |
| extracellular matrix structural | 0.801 | 4.8043204 | 6 | 0 | 0 | 0.0423389 |
| constituent | | | | | | |
| RNA binding | 0.564 | 16.226181 | 27 | 1 | −0.436683 | 3.91E−06 |
| structural molecule activity | 0.762 | 30.582787 | 38 | 1 | −0.85197 | 1.933E−07 |
| nucleic acid binding | 0.488 | 36.804271 | 64 | 5 | −3.163539 | 0.0199209 |
| cytosolic ribosome (sensu Eukarya) | 0.732 | 8.0487542 | 11 | 0 | 0 | 3.447E−07 |
| proteasome core complex (sensu | 0.564 | 2.2564559 | 4 | 0 | 0 | 0.0304081 |
| Eukarya) | | | | | | |
| eukaryotic 43S preinitiation | 0.529 | 2.1141753 | 4 | 0 | 0 | 0.036631 |
| complex | | | | | | |
| small ribosomal subunit | 0.701 | 3.5057175 | 5 | 0 | 0 | 0.0160654 |
| collagen | 0.884 | 4.4205293 | 5 | 0 | 0 | 0.0160654 |
| proteasome complex (sensu | 0.521 | 2.6060329 | 5 | 0 | 0 | 0.0301159 |
| Eukarya) | | | | | | |
| basement membrane | 0.929 | 5.5744617 | 6 | 0 | 0 | 0.0136794 |
| ribosome | 0.738 | 16.964075 | 23 | 0 | 0 | 1.114E−07 |
| ribonucleoprotein complex | 0.687 | 20.599567 | 30 | 0 | 0 | 5.336E−08 |
| chromatin | 0.541 | 5.3809737 | 7 | 1 | −1.049901 | 0.0322996 |
| cytosol | 0.603 | 14.450534 | 21 | 2 | −0.584947 | 0.0003098 |
| extracellular matrix | 0.799 | 11.577839 | 13 | 1 | −0.393003 | 0.0361871 |
| L-phenylalanine metabolism | −1.203 | 0 | 0 | 3 | −3.608402 | 0.015339 |
| phenylalanine catabolism | −1.203 | 0 | 0 | 3 | −3.608402 | 0.015339 |
| aromatic amino acid family | −1.203 | 0 | 0 | 3 | −3.608402 | 0.0246852 |
| catabolism | | | | | | |
| aromatic compound catabolism | −1.203 | 0 | 0 | 3 | −3.608402 | 0.0246852 |
| tyrosine metabolism | −1.033 | 0 | 0 | 3 | −3.099756 | 0.0246852 |
| DNA replication initiation | 0.609 | 3.0432735 | 5 | 0 | 0 | 0.0018226 |
| aromatic amino acid family | −1.037 | 0 | 0 | 4 | −4.149657 | 0.0094724 |
| metabolism | | | | | | |
| ribosome biogenesis | 0.752 | 7.5160166 | 10 | 0 | 0 | 0.0001702 |
| regulation of translation | 0.137 | 1.8846141 | 4 | 2 | −1.063299 | 0.0071406 |
| ribosome biogenesis and assembly | 0.752 | 7.5160166 | 10 | 0 | 0 | 0.0002083 |
| DNA denendent DNA replication | 0.546 | 3.2738639 | 6 | 0 | 0 | 0.0139176 |
| aromatic compound metabolism | −0.503 | 1.5973586 | 1 | 6 | −5.120159 | 0.013176 |
| posttranslational membrane | 0.491 | 4.7069693 | 5 | 2 | −1.272969 | 0.013176 |
| targeting | | | | | | |
| protein-ER targeting | 0.481 | 5.1236426 | 6 | 2 | −1.272969 | 0.0072796 |
| protein-membrane targeting | 0.491 | 4.7069693 | 5 | 2 | −1.272969 | 0.0259582 |
| protein biosynthesis | 0.610 | 18.130535 | 26 | 2 | −1.063299 | 2.836E−05 |
| translation | 0.372 | 4.7791123 | 8 | 2 | −1.063299 | 0.0249621 |
| response to pest/pathogen/parasite | 0.938 | 13.132262 | 14 | 0 | 0 | 0.0397381 |
| biosynthesis | 0.360 | 19.843752 | 30 | 9 | −5.785595 | 0.0008202 |
| cell adhesion | 0.672 | 15.366891 | 19 | 2 | −1.244973 | 0.0217328 |
| macromolecule biosynthesis | 0.560 | 19.256841 | 29 | 3 | −1.323209 | 0.0041806 |
| immune response | 0.912 | 19.157513 | 21 | 0 | 0 | 0.0255412 |
| cell organization and biogenesis | 0.697 | 20.530417 | 26 | 2 | −1.015958 | 0.0098063 |
| defense response | 0.859 | 21.468511 | 25 | 0 | 0 | 0.0220773 |
| response to biotic stimulus | 0.843 | 21.929029 | 26 | 0 | 0 | 0.0324375 |
| response to external stimulus | 0.763 | 24.757761 | 31 | 1 | −0.33857 | 0.051035 |
| cell proliferation | 0.517 | 18.235487 | 33 | 1 | −0.661095 | 0.0479313 |
| protein metabolism | 0.466 | 41.656205 | 60 | 10 | −9.069116 | 0.0221394 |
| physiological process | 0.333 | 113.38449 | 167 | 52 | −40.53305 | 0.0152323 |
| carboxylic acid metabolism | −0.547 | 0.8960719 | 2 | 15 | −10.20242 | 0.0128196 |
| organic acid metabolism | −0.547 | 0.8960719 | 2 | 15 | −10.20242 | 0.0135279 |
| cytoplasm organization and | 0.747 | 17.44005 | 20 | 2 | −1.015958 | 0.0113533 |
| biogenesis | | | | | | |
| cell growth and/or maintenance | 0.325 | 52.152783 | 78 | 25 | −18.64241 | 0.0032613 |

| | Discordant | | | | |
|---|---|---|---|---|---|
| | Total | | Total | | |
| | Expression | No Genes- | Expression | No Genes- | |
| Category | UP | UP | DOWN | DOWN | p < 0.05 |
| carboxylic acid metabolism | 0 | 0 | −5.598769 | 8 | 0.0151991 |
| organic acid metabolism | 0 | 0 | −5.598769 | 8 | 0.015667 |
| cytoplasm organization and | 2.4955781 | 5 | −1.5467431 | 4 | 0.0315753 |
| biogenesis | | | | | |
| cell growth and/or maintenance | 7.3648921 | 13 | −11.551056 | 20 | 0.0450794 |
| insulin-like growth factor binding | 1.7450831 | 2 | −1.3912086 | 2 | 0.0006866 |
| organic cation transporter activity | 0.3754932 | 1 | −1.1781775 | 2 | 0.0161759 |
| growth factor binding | 1.7450831 | 2 | −1.3912086 | 2 | 0.0027999 |
| heparin binding | 3.3125522 | 4 | −1.7921275 | 2 | 0.0002486 |
| glycosaminoglycan binding | 3.3125522 | 4 | −1.7921275 | 2 | 0.0005008 |
| cation transporter activity | 0.3754932 | 1 | −2.6061538 | 4 | 0.0466136 |
| catalytic activity | 3.9243146 | 9 | −16.911395 | 30 | 0.0306027 |
| extracellular space | 9.491228 | 12 | −7.4596714 | 12 | 0.0395413 |
| regulation of axon extension | 0.7769723 | 1 | −0.3395731 | 1 | 0.0617602 |
| one-carbon compound metabolism | 0 | 0 | −1.5503316 | 3 | 0.0287613 |
| angiogenesis | 2.53558 | 3 | −0.5766978 | 2 | 0.0023126 |
| regulation of cell growth | 1.7450831 | 2 | −1.3912086 | 2 | 0.0113371 |
| blood vessel development | 2.53558 | 3 | −0.5766978 | 2 | 0.0037461 |
| cell growth | 1.7450831 | 2 | −1.8333907 | 3 | 0.0044579 |
| cytoskeleton organization and | 2.4955781 | 5 | −0.9460864 | 3 | 0.0110569 |
| biogenesis | | | | | |
| regulation of cellular process | 1.7450831 | 2 | −2.4914104 | 4 | 0.0379138 |
| regulation of biological process | 1.7450831 | 2 | −2.4914104 | 4 | 0.0391032 |
| organelle organization and | 2.4955781 | 5 | −1.5467431 | 4 | 0.0108806 |
| biogenesis | | | | | |
| organogenesis | 6.7050688 | 8 | −2.696574 | 6 | 0.030497 |
| morphogenesis | 6.7050688 | 8 | −2.696574 | 6 | 0.0489539 |

TABLE 18
The significance of gene in the various expression groups: patterns, trends and pathways. The significance of gene in the various
expression patterns of early, late, continues, pathways and the concordant or discordant groups was analyzed
by using the chi square test (Table 1). Se methods for further explanation.

| | | Concordance: | | | | | | | |
|---|---|---|---|---|---|---|---|---|---|
| | All data | regeneration | | Discordance: | | Rest of the | | Both Early | |
| | (1325 | Vs. RCC (278 | | regeneration Vs. | | Data (964 | | & Late | |
| | genes) | genes) | | RCC (83 genes) | | genes) | | (323 genes) | |
| | Changed | Changed | P | Changed | P | Changed | P | Changed | P |
| Category | genes | genes | Value | genes | Value | genes | Value | genes | Value |
| All data | 1325 | N.A. | | N.A. | | N.A. | | N.A. | |
| Continuous expression- days 1, | 323 | 93 | 0.0001 | 20 | 0.9438 | 210 | 0.0004 | 323 | 0 |
| 2, 5 &14 (*) | | | | | | | | | |
| Early expression- days 1 & 2 | 629 | 114 | 0.0182 | 35 | 0.3757 | 480 | 0.0068 | 0 | 0 |
| (A) | | | | | | | | | |
| Late expression- days 5 &14(B) | 373 | 71 | 0.3105 | 28 | 0.2972 | 274 | 0.7706 | 0 | 0 |
| Up regulated | 802 | 209 | <0.0001 | 30 | <0.0001 | 563 | 0.0116 | 189 | 0.4317 |
| Down regulated | 523 | 69 | <0.0001 | 53 | <0.0001 | 401 | 0.0116 | 134 | 0.4317 |
| Regeneration/RCC: | 278 | 278 | 0 | 0 | <0.0001 | 0 | 0 | 93 | 0.0001 |
| Concordant | | | | | | | | | |
| Regeneration/RCC: | 83 | 0 | <0.0001 | 83 | 0 | 0 | 0 | 20 | 0.9438 |
| Disconcordant | | | | | | | | | |
| Rest of the Data | 964 | 0 | 0 | 0 | 0 | 964 | 0 | 210 | 0.0004 |
| VHL pathway | 104 | 59 | 0 | 16 | 0.0001 | 29 | 0 | 28 | 0.6094 |
| Hypoxia pathway | 95 | 35 | 0.0001 | 16 | <0.0001 | 44 | <0.0001 | 24 | 0.9325 |
| HRE target (HIF) | 17 | 4 | 0.968 | 7 | <0.0001 | 6 | 0.0012 | 2 | 0.3499 |
| IGF pathway | 37 | 9 | 0.7628 | 8 | 0.0003 | 20 | 0.0162 | 10 | 0.852 |
| Myc pathway | 136 | 55 | <0.0001 | 10 | 0.714 | 71 | <0.0001 | 39 | 0.2596 |
| p53 pathway | 262 | 80 | <0.0001 | 32 | <0.0001 | 150 | <0.0001 | 69 | 0.4568 |
| NF-kB pathway | 52 | 19 | 0.0083 | 5 | 0.4681 | 28 | 0.003 | 19 | 0.0549 |

| | | | | | UP | | Down | |
|---|---|---|---|---|---|---|---|---|
| | Early (629 | | Late (373 | | regulated | | regulated | |
| | genes) | | genes) | | (802 genes) | | (523 genes) | |
| | Changed | P | Changed | P | Changed | P | Changed | P |
| Category | genes | Value | genes | Value | genes | Value | genes | Value |
| All data | N.A. | | N.A. | | N.A. | | N.A. | |
| Continuous expression- days 1, | 0 | 0 | 0 | 0 | 189 | 0.4317 | 134 | 0.4317 |
| 2, 5 &14 (*) | | | | | | | | |
| Early expression- days 1 & 2 | 629 | 0 | 0 | 0 | 336 | <0.0001 | 293 | <0.0001 |
| (A) | | | | | | | | |
| Late expression- days 5 &14(B) | 0 | 0 | 373 | 0 | 277 | <0.0001 | 96 | <0.0001 |
| Up regulated | 336 | <0.0001 | 277 | <0.0001 | 802 | 0 | 0 | 0 |
| Down regulated | 293 | <0.0001 | 96 | <0.0001 | 0 | 0 | 523 | 0 |
| Regeneration/RCC: | 114 | 0.0182 | 71 | 0.3105 | 209 | <0.0001 | 69 | <0.0001 |
| Concordant | | | | | | | | |
| Regeneration/RCC: | 35 | 0.3757 | 28 | 0.2972 | 30 | <0.0001 | 53 | <0.0001 |
| Disconcordant | | | | | | | | |
| Rest of the Data | 480 | 0.0068 | 274 | 0.7706 | 563 | 0.0116 | 401 | 0.0116 |
| VHL pathway | 50 | 0.9788 | 26 | 0.5282 | 85 | <0.0001 | 19 | <0.0001 |
| Hypoxia pathway | 50 | 0.3478 | 21 | 0.2144 | 63 | 0.2762 | 32 | 0.2762 |
| HRE target (HIF) | 12 | 0.0936 | 3 | 0.4852 | 10 | 0.9163 | 7 | 0.9163 |
| IGF pathway | 19 | 0.7547 | 8 | 0.4775 | 25 | 0.4728 | 12 | 0.4728 |
| Myc pathway | 61 | 0.5789 | 36 | 0.7193 | 113 | <0.0001 | 23 | <0.0001 |
| p53 pathway | 112 | 0.1009 | 81 | 0.3009 | 199 | <0.0001 | 63 | <0.0001 |
| NF-kB pathway | 21 | 0.3668 | 12 | 0.5011 | 43 | 0.0014 | 9 | 0.0014 |

TABLE 19
The RRR genes in non-probabilistic GO ontologies. The comprehensive
probabilistic analysis may fail to capture many key aspects of the concordant
and discordant gene functions. Therefore, we also categorized the genes
into gene-by-gene, non-probabilistic GO.

| Gene | | RRR/ | RCC/ | |
|---|---|---|---|---|
| symbol | Gene name | Normal | Normal | Molecular Function |
| TJP2 | tight junction protein 2 | Up | Down | Guanylate kinase activity |
| HARS | histidyl tR synthetase | Down | Up | Histidine-tRNA ligase activity; ATP binding |
| IF | complement component factor i | Up | Down | Scavenger receptor activity; Trypsin activity |
| CYR61/ | cysteine rich protein 61 | Up | Down | Heparin binding; Insulin-like growth factor |
| IGFBP10 | | | | binding |
| FHIT | fragile histidine triad gene | Up | Down | Magnesium ion binding; Manganese ion |
| | | | | binding; Bis(5′-adenosyl)-triphosphatase |
| | | | | activity; Hydrolase activity |
| APOE | apolipoprotein E | Up | Down | Tau protein binding; Lipid binding; Lipid |
| | | | | transporter activity; Antioxidant activity; |
| | | | | Heparin binding; Apolipoprotein E receptor |
| | | | | binding; Beta-amyloid binding |
| EGLN1 | EGL nine homolog 1 (C. elegans) | Down | Up | Oxidoreductase activity, Oxidoreductase |
| | | | | activity, acting on paired donors, with |
| | | | | incorporation or reduction of molecular oxygen, |
| | | | | 2-oxoglutarate as one donor, and incorporation |
| | | | | of one atom each of oxygen into both donors; |
| | | | | Oxidoreductase activity, acting on single donors |
| | | | | with incorporation of molecular oxygen, |
| | | | | incorporation of two atoms of oxygen |
| CEACAM1 | CEA-related cell adhesion | Down | Up | Molecular_function unknown |
| | molecule 1 | | | |
| MT2A | Metallothionein 2 | Up | Down | Copper ion binding; Metal ion binding |
| LPL | lipoprotein lipase | Down | Up | Heparin binding; Hydrolase activity; Lipid |
| | | | | transporter activity; Lipoprotein lipase activity |
| TACSTD2 | tumor-associated calcium signal | Up | Down | Receptor activity |
| | transducer 2 | | | |
| PLAT | plasminogen activator, tissue | Up | Down | Peptidase activity; Plasminogen activator |
| | | | | activity; Trypsin activity; Chymotrypsin |
| | | | | activity; Hydrolase activity |
| C16orf5 | RIKEN cD 5730403B10 gene | Down | Up | Molecular_function unknown |
| EIF4A2 | eukaryotic translation initiation | Down | Up | ATP binding; Translation initiation factor |
| | factor 4A2 | | | activity; ATP-dependent helicase activity; DNA |
| | | | | binding; RNA binding; Hydrolase activity; |
| | | | | Nucleic acid binding |
| TCF21 | transcription factor 21 | Up | Down | DNA binding; RNA polymerase II transcription |
| | | | | factor activity |
| RALBP1 | Ral-interacting protein 1 | Up | Down | GTPase activator activity |
| HSPD1 | heat shock protein 1 (chaperonin)/ | Down | Up | Unfolded protein binding; ATP binding |
| | heat shock protein, 60 kDa | | | |
| SCP2 | sterol carrier protein 2, liver | Down | Up | Sterol carrier activity; Lipid binding |
| CTGF/ | connective tissue growth factor | Up | Down | Protein binding; Heparin binding; Insulin-like |
| IGFBP8 | | | | growth factor binding |
| CPT1A | carnitine palmitoyltransferase 1, | Down | Up | Transferase activity; Acyltransferase activity; |
| | liver | | | Carnitine O-palmitoyltransferase activity |
| PGK1 | phosphoglycerate kise 1 | Down | Up | Phosphoglycerate kinase activity; Transferase |
| | | | | activity |
| GC | group specific component | Up | Down | Actin binding; Carrier activity; Vitamin D |
| | | | | binding |
| HK1 | hexokise 1 | Down | Up | ATP binding; Kinase activity; Hexokinase |
| | | | | activity; Transferase activity |
| DCN | decorin | Up | Down | (?) |
| TOP3B | topoisomerase (D) III beta | Down | Up | DNA topoisomerase type I activity; |
| FRAP1 | FK506 binding protein 12- | Down | Up | Transferase activity; Binding; Inositol or |
| | rapamycin associated protein 1 | | | phosphatidylinositol kinase activity |
| IGFBP1 | insulin-like growth factor binding | Down | Up | Insulin-like growth factor binding |
| | protein 1 | | | |
| RTN3 | reticulon 3 | Down | Up | Molecular_function unknown |
| TM4SF3 | Mus musculus, clone MGC: 38363 | Up | Down | Signal transducer activity |
| | IMAGE: 5344986, mR, complete | | | |
| | cds | | | |
| GPC3 | glypican 3 | Up | Down | (?) |
| NR2F6 | nuclear receptor subfamily 2, group | Up | Down | Thyroid hormone receptor activity; Steroid |
| | F, member 6 | | | hormone receptor activity; Transcription factor |
| | | | | activity |
| ZNF144 | zinc finger protein 144 | Up | Down | Transcription factor activity; Ubiquitin-protein |
| | | | | ligase activity; Zinc ion binding |
| SLC1A1 | solute carrier family 1, member 1 | Up | Down | Sodium:dicarboxylate symporter activity; |
| | | | | Symporter activity; L-glutamate transporter |
| | | | | activity |
| SDC1 | syndecan 1 | Up | Down | Cytoskeletal protein binding |
| BCKDHA | branched chain ketoacid | Down | Up | 3-methyl-2-oxobutanoate dehydrogenase (2- |
| | dehydrogese E1, alpha polypeptide | | | methylpropanoyl-transferring) activity; Alpha- |
| | | | | ketoacid dehydrogenase activity; |
| | | | | Oxidoreductase activity; Oxidoreductase |
| | | | | activity, acting on the aldehyde or oxo group of |
| | | | | donors, disulfide as acceptor |
| SOD2 | superoxide dismutase 2, | Down | Up | Oxidoreductase activity; Superoxide dismutase |
| | mitochondrial | | | activity; Manganese ion binding; Manganese |
| | | | | superoxide dismutase activity; Metal ion |
| | | | | binding |
| SMC1L1 | SMC (structural maintence of | Up | Down | Chromatin binding; Protein binding; ATP |
| | chromosomes 1)-like 1 (S. cerevisiae) | | | binding; Protein heterodimerization activity; |
| | | | | ATPase activity; Microtubule motor activity |
| GRSF1 | G-rich RNA sequence binding | Down | Up | mRNA binding |
| | factor 1 (D5Wsu31e) D segment, | | | |
| | Chr 5, Wayne State University 31, | | | |
| | expressed | | | |
| AMACR | alpha-methylacyl-CoA racemase | Down | Up | Catalytic activity; Isomerase activity; Alpha- |
| | | | | methylacyl-CoA racemase activity |
| ENPP2 | ectonucleotide | Down | Up | Phosphodiesterase I activity; Transcription |
| | pyrophosphatase/phosphodiesterase 2 | | | factor binding; Endonuclease activity; |
| | | | | Hydrolase activity; Nucleic acid binding; |
| | | | | Nucleotide diphosphatase activity |
| PCTK3 | PCTAIRE-motif protein kise 3 | Down | Up | Signal transducer activity; ATP binding; |
| | | | | Transferase activity; Protein serine/threonine |
| | | | | kinase activity; Protein-tyrosine kinase activity |
| NCOA4 | nuclear receptor coactivator 4 | Down | Up | Transcription coactivator activity |
| KDR | kise insert domain protein receptor | Down | Up | Receptor activity; Transferase activity; Vascular |
| | | | | endothelial growth factor receptor activity; ATP |
| | | | | binding |
| CORO1B | coronin, actin binding protein 1B | Up | Down | Actin binding |
| WSB1 | RIKEN cD 2700038M07 gene - | Up | Down | Molecular_function unknown |
| | pending | | | |
| KIAA1049 | RIKEN cD 1100001J13 gene - | Down | Up | (?) |
| | pending | | | |
| SLC16A7 | solute carrier family 16 | Down | Up | Transporter activity; Monocarboxylate porter |
| | (monocarboxylic acid transporters), | | | activity; Pyruvate carrier activity; Symporter |
| | member 7 | | | activity |
| IGFBP3 | insulin-like growth factor binding | Down | Up | Insulin-like growth factor binding; Insulin-like |
| | protein 3 | | | growth factor binding; Metal ion binding; |
| | | | | Protein tyrosine phosphatase activator activity |
| MMP2 | matrix metalloproteise 2 | Up | Down/ | Calcium ion binding; Gelatinase A activity; |
| | | | Possible | Hydrolase activity; Zinc ion binding |
| | | | Conflict | |
| MTHFD1 | methylenetetrahydrofolate | Down | Up | Oxidoreductase activity; Hydrolase activity; |
| | dehydrogese (DP+ dependent), | | | Ligase activity; Methenyltetrahydrofolate |
| | methenyltetrahydrofolate | | | cyclohydrolase activity; ATP binding; |
| | cyclohydrolase, | | | Methylenetetrahydrofolate dehydrogenase |
| | formyltetrahydrofolate synthase | | | (NADP+) activity; Formate-tetrahydrofolate |
| | | | | ligase activity |
| PKD1 | polycystic kidney disease 1 | Down | Up | Sugar binding |
| | homolog | | | |
| MAT2A | Mus musculus, clone MGC: 6545 | Down | Up | ATP binding; Magnesium ion binding; |
| | IMAGE: 2655444, mR, complete | | | Methionine adenosyltransferase activity; |
| | cds | | | Transferase activity |
| SHMT2 | serine hydroxymethyl transferase 2 | Down | Up | Transferase activity; Glycine |
| | (mitochondrial); RIKEN cD | | | hydroxymethyltransferase activity |
| | 2700043D08 gene | | | |
| FHL1 | four and a half LIM domains 1 | Down | Up | Zinc ion binding |
| VEGF | vascular endothelial growth factor A | Down | Up | Heparin binding; Vascular endothelial growth |
| | | | | factor receptor binding; Extracellular matrix |
| | | | | binding; Growth factor activity; rotein |
| | | | | homodimerization activity |
| PAPOLA | poly (A) polymerase alpha | Down | Up | Polynucleotide adenylyltransferase activity; |
| | | | | Transferase activity; RNA binding |
| MYL6 | myosin light chain, alkali, | Up | Down | Calcium ion binding |
| | nonmuscle | | | |
| SHMT1 | serine hydroxymethyl transferase 1 | Down | Up | Glycine hydroxymethyltransferase activity; |
| | (soluble) | | | Transferase activity |
| GJB2 | gap junction membrane channel | Down | Up | Connexon channel activity |
| | protein beta 2 | | | |
| HSPH1 | heat shock protein, 105 kDa | Down | Up | ATP binding |
| PTPRB | protein tyrosine phosphatase, | Down | Up | Hydrolase activity; Transmembrane receptor |
| | receptor type, B | | | protein tyrosine phosphatase activity |
| UBE2V1 | Mus musculus, Similar to | Down | Up | Transcriptional activator activity; Ubiquitin |
| | ubiquitin-conjugating enzyme E2 | | | conjugating enzyme activity |
| | variant 1, clone MGC: 7660 | | | |
| | IMAGE: 3496088, mR, complete | | | |
| | cds | | | |
| KIF21A | kinesin family member 21A | Down | Up | ATP binding; Motor activity |
| THBS1 | thrombospondin 1 | Up | Down | Protein binding; Signal transducer activity; |
| | | | | Calcium ion binding; Structural molecule |
| | | | | activity; Endopeptidase inhibitor activity; |
| | | | | Heparin binding |
| MKNK2 | G protein-coupled receptor kise 7 | Down | Up | ATP binding; Transferase activity; Protein |
| | | | | serine/threonine kinase activity; Protein- |
| | | | | tyrosine kinase activity |
| ADD3 | adducin 3 (gamma) | Down | Up | Calmodulin binding; Structural constituent of |
| | | | | cytoskeleton |
| KlK1 | kallikrein 6 | Down | Up | Chymotrypsin activity; Peptidase activity; |
| | | | | Tissue kallikrein activity; Trypsin activity |
| ATP1B1 | ATPase, +/K+ transporting, beta 1 | Down | Up | Sodium:potassium-exchanging ATPase activity; |
| | polypeptide | | | |
| ARHE | ras homolog gene family, member E | Down | Up | GTP binding |
| PTPRO | protein tyrosine phosphatase, | Up | Down | Protein tyrosine phosphatase activity; Protein |
| | receptor type, O | | | tyrosine phosphatase activity; Receptor activity; |
| | | | | Transmembrane receptor protein tyrosine |
| | | | | phosphatase activity; Hydrolase activity |
| MEP1A | meprin 1 alpha | Down | Up | Meprin A activity; Metallopeptidase activity; |
| | | | | Astacin activity; Zinc ion binding; Hydrolase |
| | | | | activity |
| COX6C | cytochrome c oxidase, subunit VIc | Down | Up | Cytochrome-c oxidase activity; Oxidoreductase |
| | | | | activity |
| SLC22A1 | solute carrier family 22 (organic | Down | Up | Ion transporter activity; Organic cation |
| | cation transporter), member 1 | | | transporter activity; ATP binding |
| SPTLC1 | serine palmitoyltransferase, long | Down | Up | Serine C-palmitoyltransferase activity; |
| | chain base subunit 1 | | | Transferase activity; Acyltransferase activity |
| CAPNS1 | calpain, small subunit 1 | Down | Up | Calcium ion binding; Calpain activity |
| RRM1 | ribonucleotide reductase M1 | Down | Up | Oxidoreductase activity; Ribonucleoside- |
| | | | | diphosphate reductase activity |
| SAR1 | SAR1a gene homolog (S. cerevisiae) | Up | Down | GTP binding; |
| PPP2CB | protein phosphatase 2a, catalytic | Up | Down | Phosphoprotein phosphatase activity; Protein |
| | subunit, beta isoform | | | phosphatase type 2A activity; Hydrolase |
| | | | | activity; Manganese ion binding |
| AKAP2 | A kise (PRKA) anchor protein 2 | Up | Down | Kinase activity; Protein kinase A binding |
| ACOX1 | acyl-Coenzyme A oxidase 1, | Down | Up | Oxidoreductase activity; Acyl-CoA oxidase |
| | palmitoyl | | | activity; Electron donor activity |
| CD59 | CD59a antigen | Down | Up | (?) |
| CRYM | crystallin, mu | Up | Down | Ornithine cyclodeaminase activity |
| GADD45G | growth arrest and D-damage- | Down | Up/ | (?) |
| | inducible 45 gamma | | Possible | |
| | | | Conflict | |

TABLE 20
An ontology analysis of the concordant and discordant genes in
pathway dependent fashion: distinct and common ontologies.
The concordatly and discordantly differentially expressed genes
were clustered according to their regulation by the pathways of
VHL, hypoxia, HIF, IGF1, MYC, p53 and NF-κB. Functional
ontology was analysis performed (p < 0.05).

| Ontology | Concordant | Discondant |
|---|---|---|
| enzyme inhibitor activity | HYPOXIA | |
| cytosol | HYPOXIA, MYC | |
| structural molecule activity | VHL, HYPOXIA, | |
| | MYC, p53 | |
| protein biosynthesis | VHL, HYPOXIA, MYC | |
| ribosome | VHL, HYPOXIA, MYC | |
| structural constituent of | VHL, HYPOXIA, MYC | |
| ribosome | | |
| cell proliferation | VHL, MYC, p53 | |
| cell growth and/or maintenance | VHL, MYC, p53 | |
| DNA dependent DNA | VHL, MYC, p53 | |
| replication | | |
| DNA replication initiation | VHL, p53 | |
| collagen type V | VHL | |
| cell organization and biogenesis | MYC | |
| ribosome biogenesis and | MYC | |
| assembly | | |
| intracellular | MYC | |
| binding | MYC | |
| regulation of cell cycle | MYC, p53 | |
| response to stress | p53 | |
| cell communication | p53 | |
| intracellular signaling cascade | p53 | |
| protein targeting | p53 | |
| DNA dependent ATPase activity | p53 | |
| protein binding | p53 | |
| cell adhesion | NFkB | |
| secretory pathway | NFkB | |
| plasma membrane | NFkB | |
| immune response | p53, NFkB | |
| death | p53, NFkB | |
| posttranslational membrane | p53, NFkB | |
| targeting | | |
| protein-ER targeting | p53, NFkB | |
| signal transducer activity | p53 | IGF1 |
| extracellular | NFkB | IGF1 |
| protein metabolism | VHL, HYPOXIA, MYC | VHL |
| glycolysis | | HIF |
| regulation of cell growth | | HIF, IGF |
| cell growth | | HYPOXIA |
| insulin-like growth factor | | HYPOXIA, |
| binding | | HIF, IGF1 |
| extracellular space | | IGF1 |
| receptor activity | | IGF1 |
| one-carbon compound | | p53 |
| metabolism | | |
| angiogenesis | | p53, IGF1 |
| morphogenesis/organogenesis | | p53, IGF1 |
| heparin binding | | p53, IGF1 |
| ATP binding | | VHL |
| response to heat | | VHL, p53 |

A number of embodiments of the invention have been described. Nevertheless, it will be understood that various modifications may be made without departing from the spirit and scope of the invention. Accordingly, other embodiments are within the scope of the following claims.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A method of qualifying the tissue status in a subject comprising:
(a) measuring at least one biomarker in a sample from the subject, wherein the biomarker is selected from the group consisting of the markers listed in Table 9; and
(b) correlating the measurement with tissue status.

2. The method of claim 1, further comprising:
(c) managing treatment of the subject based on the status.

3. The method of claim 2, wherein managing treatment is selected from ordering more tests, performing surgery, chemotherapy, dialysis, treatment of acute organ failure, organ transplantation, wound healing treatment, and taking no further action.

4. The method of claim 2, further comprising:
(d) measuring the at least one biomarker after subject management.

5. The method of claim 1, wherein the tissue status is selected from the group consisting of the subject's risk of cancer, regeneration, tissue repair, acute organ failure, organ transplantation, the presence or absence of disease, the stage of disease and the effectiveness of treatment of disease.

6. The method of claim 5, further comprising measuring at least two biomarkers in a sample from the subject and correlating measurement of the biomarkers with renal status.

7. The method of claim 1, wherein the biomarkers are selected from Table 9.

8. The method of claim 1, wherein the biomarkers are selected from any one or more of Cluster 1-27.

9. The method of claim 1, wherein the biomarkers are selected from any one or more of discordant genes.

10. The method of claim 1, wherein the biomarkers are selected from any one or more of concordant genes.

11. The method of any one of claim 1, wherein measuring comprises:
(a) providing a nucleic acid sample from the subject; and
(c) capturing one or more of the biomarkers on a surface of a substrate comprising capture reagents that bind the biomarkers.

12. The method of claim 11, wherein the substrate is a nucleic acid chip.

13-24. (canceled)

25. A method of diagnosing renal status in a subject, comprising:
determining the pattern of expression of one or more markers listed in Table 9 in a sample from the subject, wherein a differential expression pattern of the one or more markers in a subject is indicative of cancer.

26. The method of claim 25, wherein the determining is of any one or more of Trends 1-27.

27. The method of claim 25, wherein the determining is of any one or more of clusters 1-27.

28. The method of claim 25, wherein the sample from the subject is selected from one or more of a kidney cell or cells, kidney tissue or blood cell.

29. A method comprising measuring a plurality of biomarkers in a sample from the subject, wherein the biomarkers are selected from one or more of the group consisting of Table 9 or Clusters 1-27.

30. A kit comprising:
(a) a capture reagent that binds a biomarker selected from Table 9 or Cluster 1-27 and combinations thereof; and
(b) a container comprising at least one of the biomarkers.

31-39. (canceled)

40. A method of monitoring the treatment of a subject for carcinoma, comprising:
determining one or more pre-treatment expression profiles of markers described in Table 9, in a cell of a subject;
administering a therapeutically effective amount of a candidate compound to the subject; and
determining one or more post-treatment expression profiles of markers described in Table 9, in a cell of a subject,
wherein a modulation of the expression profile indicates efficacy of treatment with the candidate compound.

41. The method of claim 40, wherein a pre-treatment expression profile of at least one discordantly or concordantly expressed gene indicates carcinoma.

42. The method of claim 40, wherein a post-treatment expression profile of at least one discordantly or concordantly expressed gene indicates the efficacy of the treatment.

43-44. (canceled)

45. A method of identification of a candidate molecule to treat renal carcinoma, comprising:
(a) contacting a cell with a candidate molecule; and
(b) detecting the expression profile of a target the cell,
wherein if the expression profile is of one or more of at least one discordantly and/or concordantly expressed gene the molecule may be useful to treat renal carcinoma.

46-50. (canceled)

51. A method of identifying a diagnostic marker comprising: a) obtaining a sample from an ischemically injured kidney; b) obtaining a sample from a normal kidney, c) identifying genes having differential expression in the ischemically injured kidney compared to the normal kidney; and d) selecting at least one gene of step c) as a diagnostic marker for the cancer.

52. (canceled)

53. A method of identifying a gene expression signature in a sample comprising determining the gene expression profile of a sample and comparing the expression profile to Trends 1-27.

54-58. (canceled)

59. A method comprising communicating to a subject a diagnosis relating to renal cancer status determined from the correlation of biomarkers in a sample from the subject, wherein said biomarkers are selected from the group consisting of the biomarkers listed in Table 9 or Clusters 1-27.

60-62. (canceled)

63. A method for modulating the renal profile a cell or group of cells comprising contacting a cell with one or more compounds identified by the software program PharmaProjects or a compound identified in the method of claim 61.

64-66. (canceled)

67. A method of treating a condition in a subject comprising administering to a subject a therapeutically effective amount of a compound which modulates a renal profile, wherein a modulation from a renal cell carcinoma profile to a tissue regeneration, tissue repair profile, or a normal profile indicates the efficacy of the treatment.

68-70. (canceled)

71. A biomarker for tissue status, comprising one or more of the transcripts listed in Table 9.

72-73. (canceled)

74. A method of qualifying the renal status in a subject comprising:
(a) measuring at least two biomarkers in a sample from the subject, wherein the biomarkers are selected from the group consisting of the markers listed in Table 9; and
(b) correlating the measurement with renal status.

75-85. (canceled)
